(19) United States
(12) Patent Application Publication
(10) Pub. No.: US 2025/0026795 A1
(43) Pub. Date: Jan. 23, 2025

(54) FUNCTIONAL AAV CAPSIDS FOR INTRAVITREAL ADMINISTRATION

(71) Applicant: SHAPE THERAPEUTICS INC., Seattle, WA (US)

(72) Inventors: Thomas PACKARD, Seattle, WA (US); Adrian Wrangham BRIGGS, Seattle, WA (US); David Jeffrey HUSS, Seattle, WA (US); Francois VIGNEAULT, Seattle, WA (US); Ronald James HAUSE, JR., Seattle, WA (US); Yue JIANG, Los Angeles, CA (US); Kevin Christopher STEIN, Kenmore, WA (US); Bora BANJANIN, Seattle, WA (US)

(21) Appl. No.: 18/714,509

(22) PCT Filed: Nov. 30, 2022
(86) PCT No.: PCT/US2022/051451

§ 371 Date: May 29, 2024

Related U.S. Application Data
(60) Provisional application No. 63/346,295, filed on May 26, 2022.
(60) Provisional application No. 63/284,962, filed on Dec. 1, 2021.

(51) Int. Cl. C07K 14/005; A61K 48/00; A61P 27/02; A61P 27/06; A61P 35/00; C12N 15/86

(52) U.S. Cl. CPC C07K 14/005; A61K 48/0075; A61P 27/02; A61P 27/06; A61P 35/00; C12N 15/86; C12N 2750/14122; C12N 2750/14143

(57) ABSTRACT

Disclosed herein are engineered AAV VP capsid polypeptides with the ability to assemble into virus particles and having improved tissue tropism to eye tissues, for example, retinal tissues, retinal pigment epithelial cells, or photoreceptor cells. The capsids are engineered using the high throughput discovery system described herein. In certain embodiments, provided herein are recombinant adeno-associated virus (AAV) VP capsid polypeptides having at least one mutation in a 581 to 589 region of the VP capsid polypeptide, corresponding to residues 581 to residue 589 of a VP1 polypeptide of SEQ ID NO: 1.

### CROSS-REFERENCE

The present application claims the benefit of U.S. Provisional Application No. 63/284,962, entitled “FUNCTIONAL AAV CAPSIDS FOR INTRAVITREAL ADMINISTRATION,” filed on Dec. 1, 2021, and U.S. Provisional Application No. 63/346,295, entitled “FUNCTIONAL AAV CAPSIDS FOR INTRAVITREAL ADMINISTRATION,” filed on May 26, 2022, which applications are each herein incorporated by reference in their entireties for all purposes.

### BACKGROUND

Recombinant adeno-associated viruses (rAAV) provide the leading platform for in vivo delivery of gene therapies. Current clinical trials employ a limited number of AAV capsids, primarily from naturally occurring human or primate serotypes such as AAV1, AAV2, AAV5, AAV6, AAV8, AAV9, AAVrh.10, AAV4rh.74, and AAVhu.67. These capsids often provide suboptimal targeting to tissues of interest, both due to poor infectivity of the tissue of interest and competing liver tropism. Increasing the dose to ensure infection of desired tissues can lead to dose-dependent liver toxicity. In addition, use of naturally-occurring capsids presents an immunological memory challenge—pre-immune patient populations are excluded from treatment and repeat dosing in a previously immune naïve patient is often not possible. Thus, there is a need for additional AAV capsids for use in gene therapy, in particular capsids that confer upon the rAAV high infectivity for specific tissues, such as ocular tissues.

### SUMMARY OF THE INVENTION

Described herein is a system for high throughput engineering of functional AAV capsids with altered tropism for various tissues, and using this system, have identified capsid variants with increased tropism for target tissues, such as eye tissues.

In various aspects, the present disclosure provides a viral protein (VP) capsid polypeptide comprising a 581 to 589 region corresponding to residues 581 to 589 of an AAV5 VP1 polypeptide, wherein the 581 to 589 region comprises a sequence of SEQ ID NO: 5014 and further comprises at least one substitution relative to SEQ ID NO: 9; and wherein the 581 to 589 region comprises one or more basic amino acid residues.

In some aspects, the 581 to 589 region comprises more basic amino acid residues than acidic amino acid residues. In some aspects, the acidic amino acid residues are selected from the group consisting of D and E. In some aspects, the basic amino acid residues are selected from the group consisting of K, R, and H.

In various aspects, the present disclosure provides a viral protein (VP) capsid polypeptide comprising a 581 to 589 region corresponding to residues 581 to 589 of an AAV5 VP1 polypeptide, wherein the 581-589 region of the VP capsid polypeptide comprises a sequence of SEQ ID NO: 5014, wherein X¹, X², X³, X⁴, X⁵, or X⁶ of SEQ ID NO: 5014, or any combination thereof is K or R.

In some aspects, X¹ of SEQ ID NO: 5014 is K or R. In some aspects, the 581 to 589 region of the VP capsid polypeptide has a sequence of any one of SEQ ID NOs: 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2343, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2185, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, or 1555.

In some aspects, X² of SEQ ID NO: 5014 is K or R. In some aspects, the 581 to 589 region of the VP capsid polypeptide has a sequence of any one of SEQ ID NOs: 752, 2144, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 1308, 1230, 3960, 1378, 2994, 3080, 2589, 3681, 51, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 778, 87, 3891, 4456, 4040, 4496, 1982, 2001, 1478, 1189, 120, 4791, 2158, 121, 803, 804, 1463, 805, 122, 2139, 2290, 1760, 2465, 123, 130, 131, 132, 4933, 808, 2336, 2026, 2204, 809, 810, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 234, 4962, 2269, 843, 235, 844, 236, 237, 4930, 845, 846, 238, 847, 1295, 3601, 3877, 1930, 1420, 1678, 1460, 285, 1626, 299, 3809, 4271, 3844, 1436, 300, 1515, 1617, 3646, 4059, 1431, 1331, 310, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 1959, 1840, 3736, 1283, 3861, 1383, 3644, 3691, 4259, 1749, 1418, 1176, 4216, 1924, 1421, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 954, 955, 2437, 4139, 1482, 3602, 4434, 3721, 1592, 1825, 1698, 1519, 1352, 4202, 4409, 1977, 1917, 2382, 3675, 1833, 3974, 419, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 3879, 2698, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 1019, 2012, 1803, 3642, 497, 1322, 1261, 4027, 3478, 3763, 2959, 1293, 2462, 517, 3197, 1595, 3413, 1675, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1050, 3515, 2219, 1433, 1323, 4301, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2324, 605, 1368, 3827, 4103, 606, 607, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 4035, 2910, 3391, 2808, 3099, 2913, 3676, 2852, 3284, 2731, 1609, 2375, 2201, 3537, 3994, 1319, 4467, 1802, 1227, 1623, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 4879, 1841, 4234, 4924, 1154, 1314, 4096, 2023, 3815, 2005, 1964, 3439, 2833, 4287, 4509, 4505, 4267, 3697, 3272, 4112, 2967, 3651, 1991, 1805, 1628, 3705, or 2250.

In some aspects, X³ of SEQ ID NO: 5014 is K or R. In some aspects, the 581 to 589 region of the VP capsid polypeptide has a sequence of any one of SEQ ID NOs: 4177, 1289, 740, 741, 18, 745, 3477, 1548, 3051, 2982, 2877, 3036, 3426, 3073, 3169, 747, 3362, 2784, 21, 3192, 23, 1638, 748, 1703, 1836, 3660, 29, 750, 1640, 33, 34, 35, 36, 37, 38, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 3327, 3178, 3006, 3252, 2991, 3470, 2888, 2572, 3418, 2642, 2519, 3363, 40, 2633, 41, 42, 43, 755, 45, 756, 46, 48, 1952, 758, 49, 3122, 2551, 3718, 3484, 3577, 759, 1230, 3960, 1378, 2994, 3080, 2589, 3681, 760, 53, 3925, 55, 3654, 56, 61, 4086, 1740, 1660, 765, 766, 62, 1966, 63, 64, 768, 771, 65, 66, 67, 68, 3816, 2599, 2706, 74, 3865, 1549, 1361, 3882, 3713, 1942, 1312, 4501, 76, 77, 1560, 4289, 3757, 79, 3593, 4043, 1806, 3908, 4397, 1615, 4415, 3723, 2006, 3754, 1470, 4360, 3945, 2854, 3523, 3669, 82, 4065, 83, 1294, 1484, 3891, 4456, 4040, 4496, 1982, 2001, 1478, 1189, 1490, 1885, 1926, 89, 90, 91, 779, 1180, 92, 785, 786, 94, 95, 1831, 1867, 4458, 97, 1970, 1936, 1590, 100, 789, 790, 1298, 101, 1688, 791, 792, 2007, 103, 794, 795, 104, 105, 106, 1849, 108, 1579, 110, 1906, 113, 1444, 1344, 116, 802, 117, 118, 121, 803, 804, 1463, 805, 122, 124, 125, 127, 807, 132, 809, 810, 133, 1978, 812, 135, 136, 137, 138, 813, 140, 815, 1326, 141, 142, 143, 144, 145, 1634, 1302, 817, 146, 818, 147, 148, 149, 151, 152, 153, 154, 155, 819, 820, 157, 159, 160, 163, 164, 165, 166, 822, 167, 168, 169, 170, 171, 823, 824, 1651, 173, 1483, 174, 175, 825, 176, 177, 826, 827, 181, 828, 182, 829, 184, 185, 186, 187, 188, 189, 830, 191, 192, 193, 194, 195, 196, 1391, 197, 200, 201, 202, 203, 204, 205, 832, 833, 206, 834, 836, 1334, 837, 3901, 3312, 2649, 1301, 211, 212, 1450, 4386, 3028, 213, 1446, 214, 215, 1896, 1522, 3490, 838, 217, 218, 219, 1612, 220, 221, 222, 839, 223, 840, 224, 226, 227, 230, 231, 842, 232, 233, 844, 236, 237, 851, 852, 853, 1536, 240, 854, 242, 243, 244, 245, 246, 247, 249, 250, 251, 252, 253, 254, 255, 857, 256, 257, 258, 259, 858, 859, 260, 261, 860, 861, 862, 262, 264, 1241, 265, 266, 866, 1477, 267, 867, 869, 870, 271, 871, 872, 272, 1252, 273, 1591, 275, 1980, 276, 876, 877, 277, 278, 279, 1892, 1539, 880, 1295, 3601, 3877, 1930, 1420, 1678, 1460, 282, 1799, 283, 1626, 1374, 287, 1620, 1290, 1354, 888, 291, 1956, 889, 292, 4324, 293, 4138, 891, 3903, 297, 3829, 3809, 4271, 3844, 1436, 1515, 1617, 1961, 304, 306, 1589, 4370, 308, 309, 894, 3646, 4059, 1431, 1331, 312, 895, 1779, 313, 314, 315, 316, 896, 1558, 1552, 898, 321, 322, 899, 1958, 901, 325, 902, 326, 1491, 329, 330, 905, 4484, 1756, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 1888, 4248, 1557, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 3435, 3482, 3020, 2748, 2646, 2787, 2760, 2563, 3085, 3133, 3158, 3170, 2703, 3494, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 2979, 2542, 1762, 4472, 1472, 1529, 4057, 1976, 4507, 1583, 338, 911, 1711, 340, 912, 2013, 341, 4440, 917, 344, 918, 919, 921, 922, 347, 351, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 4145, 354, 925, 1782, 926, 927, 3610, 357, 359, 4224, 1918, 1959, 3736, 1770, 931, 4155, 364, 1279, 365, 1543, 367, 4049, 1633, 1722, 368, 369, 1495, 1566, 370, 1211, 1873, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 3741, 1938, 3709, 1727, 3893, 1280, 3622, 1784, 3834, 1752, 1983, 1743, 3979, 4461, 4113, 3817, 4232, 1340, 1489, 4214, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 377, 1395, 4453, 3940, 3778, 4374, 4210, 4009, 4264, 4339, 4368, 1738, 378, 1610, 4194, 4474, 4013, 4266, 1771, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 2002, 1657, 1663, 1336, 3894, 1554, 1494, 1405, 3576, 4314, 3695, 1851, 4362, 1815, 3845, 4511, 3656, 3638, 1406, 1596, 4454, 1443, 1661, 1359, 1734, 3650, 1910, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1680, 4441, 3628, 3725, 1945, 4206, 1588, 1439, 3794, 4447, 1838, 3776, 3658, 4064, 1377, 4011, 4272, 4503, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 3613, 3273, 3452, 3525, 1229, 4105, 3835, 383, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 1520, 1767, 1466, 1194, 4137, 1908, 1927, 1758, 3772, 3661, 4186, 4174, 1170, 3850, 1315, 1611, 1530, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 1716, 3961, 1506, 4221, 1376, 1683, 4276, 1445, 1721, 1632, 3607, 1328, 1455, 1913, 1497, 1317, 1951, 1778, 1379, 3768, 4225, 1668, 390, 391, 4406, 4341, 1480, 395, 1714, 1422, 396, 397, 3618, 4433, 1177, 3898, 398, 949, 950, 399, 951, 400, 1965, 4471, 952, 402, 2475, 403, 404, 4139, 1482, 3602, 4434, 3721, 1592, 1519, 1352, 4202, 4409, 1977, 1917, 407, 408, 1631, 1684, 409, 3889, 958, 959, 412, 960, 414, 415, 961, 1905, 416, 963, 417, 3675, 1833, 3974, 420, 421, 965, 966, 969, 970, 422, 1897, 423, 4133, 3830, 972, 424, 973, 427, 1708, 4141, 3749, 431, 1619, 975, 4044, 3590, 1944, 4126, 1923, 1706, 4400, 434, 1606, 979, 2604, 2895, 436, 3448, 1692, 983, 3862, 4055, 4358, 984, 985, 1664, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 3535, 1306, 4084, 3282, 1780, 987, 988, 990, 451, 452, 991, 992, 1728, 994, 995, 454, 1696, 3116, 1869, 456, 1386, 999, 3879, 2698, 1001, 461, 1447, 462, 464, 4425, 1645, 1231, 1468, 472, 473, 476, 1273, 1007, 1744, 1488, 1659, 477, 1604, 481, 4313, 482, 483, 1499, 484, 1016, 4073, 1777, 1458, 4274, 487, 1775, 4205, 1526, 1697, 1017, 2000, 489, 3917, 3616, 4311, 1800, 4473, 4085, 4401, 1310, 3783, 3765, 491, 1948, 3699, 1735, 3792, 4290, 495, 4090, 496, 2012, 1803, 3642, 1322, 498, 1021, 1022, 502, 1333, 503, 1023, 1890, 510, 1026, 3608, 1732, 511, 2397, 1864, 1816, 1531, 3636, 1699, 1027, 1029, 1366, 2112, 1261, 4027, 3478, 3763, 2959, 517, 3197, 1595, 3413, 1448, 518, 1675, 1797, 520, 1999, 3759, 1215, 4122, 1730, 524, 526, 3966, 3911, 529, 3289, 1821, 530, 531, 1874, 1038, 536, 537, 3767, 1929, 1041, 1042, 3683, 542, 1338, 4331, 4332, 4466, 2719, 3803, 4123, 4233, 1824, 3129, 3087, 3505, 4158, 4254, 1449, 3720, 4351, 4414, 1673, 3453, 3254, 1649, 546, 4240, 3580, 1046, 1047, 550, 2598, 551, 552, 3819, 1259, 4062, 2841, 1050, 3515, 4301, 1052, 1053, 3738, 1567, 1270, 3857, 1055, 1257, 1238, 3662, 561, 562, 1058, 4203, 1899, 3981, 1062, 1303, 4116, 567, 1946, 3620, 1514, 4002, 3679, 3964, 3707, 3666, 1348, 4273, 1804, 4497, 1401, 4438, 1895, 1264, 572, 1912, 3939, 4170, 4056, 1271, 1065, 1521, 1221, 1451, 1654, 3934, 4080, 4352, 1700, 3739, 1565, 1419, 1811, 3950, 1690, 1397, 3716, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 1723, 1070, 3750, 3633, 4070, 4104, 4162, 4235, 1863, 4346, 1881, 1814, 1572, 4443, 3761, 1346, 1587, 1072, 3592, 576, 1891, 4469, 1275, 1950, 1963, 4129, 1296, 1551, 3733, 4111, 3993, 4108, 3949, 1287, 1713, 1736, 1995, 1435, 1424, 1830, 1705, 1861, 1197, 1254, 4120, 1545, 4417, 1469, 1785, 1236, 4293, 3839, 3983, 4395, 1957, 3928, 4295, 3715, 3855, 3609, 1990, 4076, 4175, 3927, 1757, 1650, 4299, 1773, 2004, 1267, 1364, 1462, 1879, 4007, 3729, 3665, 3722, 1691, 1624, 1751, 1394, 3924, 3952, 580, 581, 2003, 1343, 1559, 1250, 1555, 4114, 1818, 1075, 1076, 1077, 1704, 4448, 1078, 1079, 1786, 585, 3354, 1832, 3432, 2891, 2505, 1903, 1081, 1082, 4459, 1083, 1563, 590, 591, 1085, 594, 1453, 2559, 2590, 2738, 4478, 4069, 1809, 1327, 4150, 3260, 3481, 3672, 3625, 2008, 4439, 3852, 3997, 1411, 599, 3847, 1268, 4050, 3377, 4424, 1496, 4143, 4493, 3492, 1962, 600, 601, 4017, 4477, 1794, 4263, 3072, 2009, 1754, 1087, 1088, 3888, 1368, 3827, 4103, 606, 1090, 1571, 1091, 1438, 1092, 1093, 612, 1094, 3701, 4389, 613, 1603, 1332, 1715, 4369, 1652, 1096, 617, 1541, 618, 1921, 1733, 1373, 1870, 619, 4218, 1798, 1100, 1702, 1101, 1667, 1384, 623, 3700, 1788, 1600, 624, 626, 2771, 627, 1339, 3875, 628, 629, 3070, 4860, 2557, 2795, 2906, 3118, 1614, 1947, 4479, 3766, 632, 1104, 1105, 633, 1813, 636, 3706, 1110, 640, 1111, 641, 642, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 3095, 1286, 3624, 4481, 1432, 3511, 2735, 4381, 3295, 1585, 1114, 1766, 3221, 2643, 2739, 3920, 644, 1116, 645, 646, 647, 1801, 4279, 3762, 1637, 1509, 1119, 2965, 2369, 2695, 3293, 650, 651, 1120, 652, 1121, 3264, 655, 658, 659, 4035, 2910, 3391, 2808, 3099, 2913, 3676, 2852, 3284, 2731, 1609, 3994, 1319, 4467, 1802, 1227, 1623, 662, 663, 664, 1124, 1125, 1128, 1129, 668, 669, 1130, 670, 1131, 1527, 1132, 671, 1772, 1135, 672, 1933, 1731, 1793, 675, 676, 3342, 1138, 1139, 677, 679, 1142, 1143, 681, 1145, 682, 685, 3822, 689, 690, 691, 1147, 1601, 3905, 694, 695, 1414, 1150, 1795, 1372, 1695, 1866, 4115, 1719, 698, 1920, 699, 700, 1544, 4305, 1636, 1393, 2010, 3810, 4452, 3677, 4227, 1277, 1400, 4402, 4157, 4197, 1337, 1253, 1532, 4028, 3989, 1493, 1854, 1902, 1510, 4182, 706, 707, 708, 4476, 1245, 714, 1841, 4234, 1314, 4096, 717, 1155, 4217, 1156, 723, 1157, 1627, 725, 1159, 1953, 1160, 1201, 727, 3787, 3747, 730, 1846, 3815, 2005, 1964, 1822, 1262, 4480, 1537, 1291, 1318, 4294, 1582, 734, 3439, 2833, 4287, 4509, 4505, 4267, 3697, 3272, 4112, 2967, 3651, 1628, 3705, 1195, 3859, 1164, 1165, 1166, 1745, 1973, 1168, 3807, 4307, 1454, or 1781.

In some aspects, X⁴ of SEQ ID NO: 5014 is K or R. In some aspects, the 581 to 589 region of the VP capsid polypeptide has a sequence of any one of SEQ ID NOs: 739, 4177, 1289, 15, 743, 3477, 3051, 2982, 2877, 746, 3036, 3426, 3073, 3169, 3362, 2784, 3192, 1638, 25, 1703, 1836, 3660, 30, 32, 752, 1653, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 1467, 3327, 3178, 3006, 3252, 2991, 3470, 2888, 2572, 3418, 2642, 2519, 3363, 2633, 757, 1952, 3122, 2551, 3718, 2216, 3484, 3577, 1308, 1230, 3960, 2994, 3080, 2589, 3681, 51, 761, 3925, 3654, 763, 764, 57, 4086, 1740, 767, 772, 2599, 2706, 70, 3865, 1549, 1361, 3882, 1942, 1312, 4501, 1413, 3593, 4043, 1806, 3908, 4397, 1615, 2006, 3945, 2854, 3523, 3669, 1356, 777, 4065, 84, 1484, 778, 87, 3891, 4456, 4040, 4496, 2001, 1478, 1189, 1490, 1885, 2270, 1180, 786, 4458, 1970, 1590, 98, 1298, 101, 1688, 794, 107, 1849, 797, 1579, 111, 112, 113, 1444, 801, 114, 115, 119, 120, 122, 1760, 123, 124, 806, 125, 126, 127, 128, 129, 130, 134, 811, 814, 139, 1326, 144, 816, 1302, 150, 152, 821, 160, 161, 162, 1651, 1483, 179, 180, 183, 185, 187, 1391, 198, 199, 831, 204, 833, 207, 3901, 3312, 2649, 1301, 4386, 3028, 1896, 1522, 3490, 216, 218, 1612, 225, 228, 229, 234, 239, 849, 850, 1536, 241, 246, 248, 855, 249, 252, 253, 261, 4966, 864, 263, 4749, 2296, 1241, 866, 268, 868, 269, 270, 873, 874, 1252, 273, 1591, 274, 1980, 878, 1539, 3601, 3877, 1930, 1420, 1678, 1460, 1799, 1374, 1290, 1354, 290, 2391, 4324, 294, 4138, 3903, 892, 3829, 298, 3844, 1436, 300, 1515, 1617, 1961, 303, 1589, 893, 4370, 3646, 4059, 1331, 1558, 1552, 320, 1958, 1491, 1635, 332, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3414, 3093, 2996, 3141, 3544, 3102, 2856, 2957, 3466, 3461, 2788, 2660, 3344, 3204, 2720, 3379, 2826, 2785, 3249, 3527, 3352, 2569, 4457, 2011, 3906, 1533, 1755, 1888, 4248, 1557, 3804, 1363, 1934, 1602, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 1852, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 1911, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 2662, 2875, 3223, 2878, 3285, 2757, 3057, 2951, 3519, 2579, 2853, 3387, 3401, 3493, 3393, 1608, 3365, 2885, 3574, 3081, 3007, 3568, 3152, 3367, 3358, 3468, 3290, 3550, 3132, 2726, 2879, 3190, 3309, 2613, 2765, 3454, 2851, 2881, 3232, 3458, 3326, 3353, 2927, 3212, 2950, 3376, 3208, 3062, 3612, 3195, 3385, 2686, 2602, 3569, 3123, 2727, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 2666, 3246, 3130, 3560, 3236, 2730, 3572, 1607, 3689, 2968, 1457, 1500, 1184, 3450, 2577, 2567, 2532, 3415, 2934, 2984, 2568, 4147, 3002, 2986, 910, 2979, 2542, 4472, 1472, 4057, 4507, 1711, 913, 916, 1594, 4440, 343, 918, 345, 348, 350, 1518, 3728, 3637, 3570, 3429, 2870, 2995, 4338, 3205, 2880, 3261, 1994, 3968, 4291, 4204, 4071, 352, 2820, 3912, 3287, 1218, 1190, 4145, 353, 355, 1782, 928, 3610, 358, 4224, 1918, 1959, 1840, 3736, 4155, 1543, 4049, 1633, 1495, 372, 1873, 1726, 1748, 4411, 4506, 4270, 4156, 3780, 4036, 4030, 4016, 1550, 3899, 1247, 1244, 4140, 3930, 4117, 4189, 4001, 3799, 3643, 3619, 4149, 1313, 3709, 1727, 3893, 1280, 3622, 1784, 3834, 1752, 3979, 4461, 4113, 3817, 4232, 3902, 1857, 1904, 1481, 1516, 3820, 3789, 4252, 4047, 4330, 4082, 4094, 3785, 3687, 4318, 4243, 3866, 1621, 4315, 1192, 1513, 1856, 3324, 2769, 2678, 3296, 375, 1395, 1584, 4093, 3940, 3778, 4374, 4210, 4009, 4264, 4368, 1610, 4194, 4474, 4013, 4266, 1759, 1922, 3663, 1642, 1185, 4356, 3932, 1233, 4212, 3858, 1198, 4192, 3790, 3712, 1969, 934, 1790, 1663, 1336, 3894, 1554, 1494, 1405, 3576, 4314, 3695, 3845, 4511, 3656, 1406, 1596, 4504, 1359, 1734, 3650, 1305, 1208, 1988, 4451, 1282, 4380, 1658, 1452, 1687, 3791, 4491, 4323, 4014, 1304, 1440, 4422, 3861, 1383, 3644, 3691, 4259, 1749, 4216, 1924, 1421, 1387, 3992, 4423, 4253, 1618, 1871, 1256, 3748, 1297, 1586, 1251, 1299, 1954, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 3627, 3777, 1371, 3685, 1325, 1940, 4482, 4258, 3812, 1581, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4153, 2707, 3730, 4426, 4180, 4041, 3923, 4444, 3851, 4429, 4361, 1441, 4394, 4302, 3758, 4510, 3742, 1666, 1407, 4187, 1188, 1724, 1646, 4280, 1613, 4006, 1984, 1351, 3653, 3833, 2561, 4442, 3900, 4298, 1203, 4441, 3628, 3725, 937, 4447, 3776, 3658, 4064, 1324, 1367, 3594, 3680, 1300, 1987, 1862, 3998, 1380, 4185, 3686, 3696, 4463, 1350, 4008, 4230, 3853, 4244, 4083, 1546, 3613, 3273, 3452, 3525, 4105, 3835, 1263, 4020, 4144, 1224, 1199, 3667, 2778, 4412, 4407, 1812, 1810, 3823, 1416, 4333, 3146, 4281, 4211, 1979, 1456, 1239, 4167, 3740, 1876, 1388, 1967, 1474, 1471, 4176, 1232, 1989, 4125, 1741, 3585, 1174, 1396, 4061, 4015, 1207, 3698, 3825, 3922, 4021, 4181, 1598, 1916, 1429, 4502, 4231, 1321, 4390, 3693, 3734, 3635, 1768, 4025, 3976, 3840, 3963, 4286, 1502, 3836, 4161, 3659, 3743, 4130, 3605, 1385, 1783, 4392, 3826, 1473, 1883, 1834, 3984, 4495, 3910, 4160, 3671, 4184, 4132, 4250, 1503, 1237, 1523, 1417, 4063, 1562, 3977, 1971, 1707, 3769, 4074, 1534, 4239, 4003, 3915, 1689, 1776, 1410, 1823, 1498, 1329, 386, 3936, 4054, 4260, 4359, 4131, 4297, 4357, 4022, 3770, 4046, 1222, 3953, 4228, 4226, 4277, 1520, 1767, 1466, 1194, 4137, 1908, 1927, 1758, 3772, 3661, 4186, 4174, 1170, 1859, 4183, 1408, 1403, 941, 4033, 3623, 1826, 3719, 1931, 3584, 4004, 3591, 3967, 4168, 3957, 3878, 4165, 4191, 3980, 4058, 4200, 4336, 4322, 3991, 4106, 1641, 1570, 3978, 4154, 1365, 4257, 3846, 4345, 4379, 3603, 1693, 3937, 3801, 4201, 1898, 1845, 1226, 1220, 3745, 3988, 3975, 4236, 4075, 4146, 4256, 4308, 4000, 4148, 3588, 3868, 4403, 3883, 4326, 4393, 3895, 4237, 3955, 3727, 4005, 4489, 3982, 4437, 3597, 4342, 3872, 3987, 4490, 3876, 3668, 4300, 4042, 3648, 3996, 3800, 4367, 4099, 1807, 3887, 4428, 1884, 1682, 4364, 4306, 3869, 3600, 3914, 1968, 1670, 3811, 1865, 3961, 1506, 4221, 1376, 1379, 3768, 4225, 945, 394, 1714, 1422, 3618, 4433, 1965, 4471, 954, 4139, 1482, 3721, 1825, 1698, 1519, 1352, 4202, 4409, 1977, 1917, 405, 406, 3889, 413, 962, 418, 3675, 3974, 964, 420, 967, 968, 1897, 1272, 4133, 425, 4141, 430, 3749, 4044, 3590, 1944, 4126, 977, 4400, 1606, 980, 2604, 2895, 3448, 4563, 1692, 4055, 4358, 1664, 4309, 4045, 3854, 3682, 2688, 4366, 3425, 3231, 3649, 2759, 2724, 3873, 3755, 1216, 2754, 2606, 3471, 3234, 2565, 2803, 2562, 2684, 3411, 3405, 2690, 1255, 3985, 3535, 1306, 4084, 3282, 447, 1353, 1780, 449, 1485, 453, 1728, 454, 1696, 455, 3116, 1869, 457, 1386, 3879, 2698, 459, 1447, 4425, 1645, 467, 1488, 478, 1604, 1011, 4313, 4073, 4274, 1775, 2000, 2480, 1882, 1817, 1880, 1800, 4473, 3783, 3870, 1948, 3699, 1735, 3792, 4290, 492, 4090, 1019, 1803, 3642, 497, 500, 1333, 1890, 1024, 3608, 1864, 1531, 3636, 1699, 1028, 1366, 1030, 4027, 3478, 3763, 2959, 1293, 517, 3197, 1595, 3413, 2238, 1031, 1448, 1032, 1675, 1797, 3759, 1215, 4122, 3966, 528, 3911, 3289, 1821, 1874, 1037, 536, 1039, 3767, 3683, 540, 541, 1338, 1616, 1901, 1517, 2719, 3803, 4123, 4233, 3129, 3087, 3505, 4158, 1044, 3720, 4351, 4414, 1673, 3453, 3254, 1649, 543, 4240, 3580, 2598, 4062, 2841, 1049, 3515, 1433, 1323, 4301, 1051, 3738, 1270, 3857, 1056, 3662, 1057, 3981, 1063, 568, 570, 3620, 1064, 4002, 3964, 3707, 3666, 1348, 4273, 4497, 1401, 4438, 1895, 1912, 3939, 4170, 4056, 1221, 1654, 1067, 3934, 4080, 4352, 574, 3739, 1565, 3950, 1397, 3716, 1569, 1465, 4268, 1183, 3726, 3881, 4134, 4347, 4012, 3938, 4066, 4198, 4416, 4164, 3630, 4325, 4375, 4371, 4432, 4215, 1753, 3944, 1409, 4195, 4455, 4512, 3750, 3633, 4070, 4104, 4162, 4235, 4346, 1572, 4443, 3761, 1346, 3592, 1891, 4469, 1275, 577, 1963, 4129, 1296, 1360, 3733, 4111, 3993, 4108, 3949, 1556, 1435, 1424, 1830, 1197, 1254, 4120, 1545, 4417, 1469, 1292, 4293, 1827, 3839, 3983, 4395, 4295, 3715, 3855, 3609, 4076, 4175, 3927, 1650, 4299, 1773, 2004, 1267, 1364, 4007, 3729, 3665, 3722, 1712, 1751, 3924, 3952, 1343, 4114, 1704, 4448, 1786, 3354, 586, 3432, 2891, 587, 1903, 2077, 1080, 1563, 2274, 595, 2559, 2590, 2738, 4478, 4069, 1327, 4150, 3260, 3481, 3672, 3625, 2008, 4439, 3852, 3997, 1411, 1268, 4050, 3377, 4424, 1496, 4143, 4493, 3492, 1962, 1412, 4017, 4477, 1794, 4263, 3072, 2199, 3888, 604, 605, 1368, 3827, 4103, 607, 1571, 1438, 3701, 4389, 4897, 1603, 2154, 1715, 4369, 1652, 1733, 4218, 1798, 621, 1667, 1384, 3700, 1788, 1600, 1102, 1423, 2771, 1339, 3070, 2557, 2795, 2906, 3299, 3118, 4779, 2028, 1614, 1947, 630, 4479, 3766, 1106, 634, 1108, 635, 638, 3706, 3735, 3390, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 1269, 3095, 1286, 3624, 4481, 3511, 2735, 4381, 3295, 1585, 3221, 2643, 2739, 3920, 1801, 4279, 2502, 1117, 2965, 2695, 3293, 3264, 2076, 657, 2910, 3391, 2808, 3099, 2913, 3676, 2852, 3284, 2731, 1609, 3537, 3994, 1319, 4467, 1802, 1227, 1623, 667, 1133, 1134, 673, 1933, 1731, 1793, 3342, 683, 1146, 687, 3822, 1601, 692, 3905, 1414, 696, 1695, 4115, 701, 1544, 702, 703, 4031, 2010, 3810, 4452, 3677, 4227, 4402, 4157, 4197, 1337, 1253, 1532, 1493, 1854, 1510, 705, 1153, 4476, 1375, 712, 1245, 715, 4234, 1154, 1314, 4096, 720, 1311, 721, 722, 4217, 726, 3815, 2005, 1964, 1262, 4480, 4294, 1582, 3439, 2833, 4287, 4509, 3272, 4112, 2967, 3651, 1991, 1805, 1628, 3705, 1195, 3859, 3807, 4307, 1454, or 1781.

In some aspects, X⁵ of SEQ ID NO: 5014 is K or R. In some aspects, the 581 to 589 region of the VP capsid polypeptide has a sequence of any one of SEQ ID NOs: 2498, 4177, 742, 16, 17, 18, 745, 3477, 1548, 2982, 3426, 3073, 3169, 747, 3362, 4942, 23, 28, 2038, 3660, 750, 31, 32, 1640, 34, 37, 4925, 1205, 4288, 4053, 3263, 2553, 3947, 2710, 2608, 3467, 2796, 2961, 2529, 3320, 2674, 2635, 3446, 3053, 3688, 4320, 3708, 2807, 3562, 3274, 3374, 3013, 3416, 2981, 3065, 2777, 3469, 2977, 2638, 2972, 3059, 2574, 3380, 4262, 3167, 3368, 1225, 3178, 3006, 3252, 2991, 2642, 2519, 3363, 2633, 42, 755, 44, 756, 46, 1952, 758, 2410, 2551, 3718, 1308, 1230, 3960, 1378, 2589, 3681, 760, 53, 4951, 4591, 762, 3925, 55, 3654, 763, 60, 61, 4086, 1740, 766, 1966, 63, 2041, 64, 769, 770, 2384, 771, 65, 772, 2197, 68, 3816, 69, 2599, 70, 3865, 1549, 3882, 75, 4501, 76, 774, 1413, 776, 3757, 3593, 4043, 4397, 4415, 3754, 1470, 4360, 3945, 2854, 3523, 3669, 1356, 81, 82, 4065, 83, 84, 1294, 87, 3891, 4456, 4040, 1189, 1490, 90, 92, 782, 94, 1831, 1867, 4458, 1936, 100, 792, 2007, 4556, 795, 104, 106, 1849, 2509, 110, 1906, 798, 2359, 2461, 4864, 1344, 116, 120, 121, 803, 1463, 805, 1760, 123, 124, 806, 127, 2085, 130, 132, 809, 1978, 134, 813, 139, 815, 1326, 141, 142, 145, 4816, 1302, 817, 146, 818, 148, 149, 4546, 150, 153, 156, 157, 163, 164, 166, 169, 823, 824, 1483, 175, 177, 178, 179, 826, 827, 182, 183, 829, 184, 185, 190, 191, 192, 2361, 195, 1391, 197, 2223, 201, 202, 4775, 1334, 837, 3901, 3312, 212, 4386, 3028, 213, 1446, 214, 215, 1896, 3490, 217, 219, 223, 225, 2088, 4542, 226, 227, 230, 843, 844, 237, 845, 239, 849, 852, 1536, 854, 244, 245, 248, 855, 251, 253, 255, 257, 858, 860, 262, 863, 4535, 865, 264, 1241, 265, 1477, 267, 867, 1252, 273, 275, 1980, 277, 281, 1892, 879, 1539, 881, 1295, 282, 285, 1626, 2082, 1374, 1620, 4564, 289, 887, 888, 889, 4324, 293, 4138, 296, 3903, 892, 3829, 298, 301, 302, 1589, 4370, 2342, 309, 4059, 310, 1779, 1558, 1552, 317, 320, 898, 321, 2016, 2134, 324, 325, 327, 1635, 4484, 1756, 4609, 332, 4635, 3069, 2637, 3025, 2782, 3247, 3350, 2692, 2586, 3214, 3163, 2541, 3090, 3097, 3462, 3037, 3419, 2780, 2931, 1276, 2789, 3228, 2657, 3189, 2975, 3346, 3067, 2728, 2955, 3408, 3027, 3094, 3203, 2988, 2751, 2953, 2566, 2578, 2901, 2837, 2822, 2745, 3473, 3530, 3040, 2597, 3509, 3522, 3161, 2926, 3209, 3105, 3400, 2630, 2905, 2671, 2818, 2806, 2534, 2773, 2623, 3491, 3243, 3136, 2693, 3164, 2679, 3510, 3110, 2672, 3442, 2929, 3184, 3488, 3102, 3466, 3461, 2788, 2660, 3344, 2720, 3379, 2826, 3249, 3527, 4460, 3352, 4319, 3906, 4248, 3804, 1363, 1404, 4349, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3782, 3629, 3578, 3885, 4081, 1764, 1852, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 4222, 1504, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3909, 4269, 3019, 1415, 3020, 2748, 2646, 2787, 2760, 3158, 3170, 2703, 3494, 3404, 2675, 2749, 3341, 3355, 2594, 3495, 2884, 3516, 3042, 2737, 2923, 3563, 2855, 3476, 2611, 2937, 3383, 2875, 3223, 2878, 1808, 2951, 3519, 2579, 2853, 3387, 3401, 3007, 3568, 3152, 3367, 3358, 336, 3290, 3550, 3309, 2613, 2765, 1355, 2881, 3232, 3458, 3326, 3353, 2927, 3212, 2950, 3376, 3208, 3062, 3612, 3195, 3385, 2686, 1622, 3361, 3022, 3427, 3107, 3140, 3371, 3276, 2831, 3501, 2924, 3366, 3199, 3101, 3321, 3499, 2917, 2683, 1464, 3560, 3236, 2730, 3572, 1607, 909, 2577, 2567, 2532, 3415, 2934, 2984, 2568, 3002, 2338, 4472, 1472, 1529, 4507, 1583, 340, 912, 2013, 913, 2277, 1594, 4440, 917, 343, 344, 345, 346, 350, 351, 4498, 3637, 3570, 3429, 4091, 4338, 3205, 2880, 3261, 3968, 4291, 4204, 4034, 2820, 3912, 3287, 1218, 1190, 4145, 354, 3610, 4224, 1840, 3736, 1770, 4155, 932, 363, 1279, 2156, 1722, 368, 1566, 370, 4967, 372, 1211, 2494, 1726, 1748, 1191, 4039, 4411, 4506, 4270, 4156, 3780, 1550, 3899, 1181, 3919, 1639, 3664, 4189, 4001, 3799, 1240, 1686, 1479, 1307, 1575, 4296, 1665, 3871, 1605, 4408, 4149, 3741, 3893, 1280, 1983, 3979, 3817, 1489, 4214, 3902, 1857, 1904, 3820, 3789, 4252, 4330, 4082, 3785, 3687, 4243, 3704, 4172, 4315, 1192, 2769, 2678, 3296, 2343, 4093, 4453, 3940, 3778, 4374, 4339, 4368, 4194, 4474, 4013, 1771, 1759, 3860, 3832, 1381, 4356, 1233, 4212, 3858, 1198, 3611, 1919, 1819, 3790, 1969, 4247, 1790, 3894, 1554, 3576, 4314, 1851, 1850, 4511, 3656, 3638, 1406, 1443, 1661, 4504, 3831, 4435, 1208, 1988, 3849, 1835, 4380, 4024, 4491, 4323, 4014, 1511, 1981, 1676, 1547, 1383, 3644, 3691, 4259, 1176, 4216, 1924, 1387, 4423, 1256, 4119, 1251, 3990, 4029, 4097, 3890, 3796, 4343, 1202, 4413, 4384, 3808, 3892, 3926, 3756, 4405, 4109, 1210, 4492, 3969, 4436, 3175, 4128, 2970, 1228, 3534, 3437, 3784, 4470, 4010, 4334, 3821, 4171, 3824, 3631, 4494, 1320, 1204, 1223, 1284, 1685, 3760, 3753, 1178, 3884, 2783, 4038, 3545, 2916, 3112, 2834, 3880, 3673, 4102, 4100, 4327, 3634, 3617, 4445, 4303, 1370, 3838, 1187, 3615, 3746, 3935, 1941, 4419, 1171, 3670, 3777, 936, 3685, 1325, 1940, 4482, 4258, 3812, 4124, 4508, 4500, 3652, 3972, 1630, 4421, 1172, 4026, 2740, 3125, 3546, 4513, 4340, 1763, 1487, 3589, 3907, 4041, 3851, 4077, 4187, 1193, 1196, 1724, 1717, 1200, 1975, 1613, 4006, 3653, 3833, 1593, 4442, 3900, 4388, 1820, 4317, 4249, 3628, 3794, 4447, 3776, 3658, 1377, 4011, 4272, 3594, 4185, 3686, 3696, 1350, 4230, 3853, 4244, 1909, 3897, 1546, 3273, 3452, 3525, 4105, 3835, 383, 1263, 1492, 3856, 3586, 1175, 4144, 1224, 1199, 3667, 2778, 4412, 1507, 3146, 4281, 1179, 3843, 3740, 1388, 1474, 1342, 1471, 4176, 1741, 1508, 4378, 1212, 4015, 1207, 3698, 3825, 4231, 3806, 3896, 3963, 4286, 3743, 4130, 3605, 4173, 4265, 1246, 1737, 3826, 1473, 1992, 1883, 1656, 3671, 4063, 1847, 4239, 3915, 1265, 1498, 1329, 3874, 4261, 1791, 3579, 4359, 4131, 4297, 4357, 4046, 3788, 4169, 1925, 4228, 4101, 1889, 1369, 4088, 4019, 1194, 3772, 3850, 1315, 1530, 1859, 4183, 1408, 4033, 3623, 1928, 1459, 1765, 4004, 3591, 3967, 4168, 3957, 3878, 3980, 3958, 4051, 3798, 4032, 3991, 4106, 1641, 4464, 3813, 4257, 3846, 4345, 1787, 1214, 4483, 4201, 1220, 3867, 3640, 3916, 1209, 4236, 4075, 4146, 4256, 4308, 4000, 4148, 3588, 3883, 4326, 4092, 4283, 1553, 3805, 4151, 1281, 1681, 4489, 3982, 4437, 3597, 4342, 3987, 3583, 3647, 389, 4304, 3793, 1937, 3996, 3800, 4367, 4099, 1390, 4364, 4306, 1278, 1672, 1542, 1915, 3600, 3948, 3811, 1362, 4276, 1445, 1721, 1632, 3607, 1317, 1778, 1668, 391, 4406, 4341, 1480, 393, 394, 395, 946, 3618, 4433, 1177, 947, 950, 400, 4471, 401, 402, 954, 3602, 4434, 3721, 1825, 4202, 4409, 406, 1631, 1684, 3889, 410, 412, 2352, 1905, 417, 418, 3675, 1833, 3974, 419, 964, 420, 421, 969, 4733, 1272, 971, 423, 3830, 973, 426, 1708, 428, 4141, 3749, 1619, 975, 4044, 3590, 1706, 4400, 979, 2895, 3448, 438, 982, 2433, 983, 442, 3862, 2435, 443, 4309, 4045, 3854, 3682, 444, 445, 4366, 3425, 3231, 3649, 2759, 2724, 3873, 3599, 3775, 3471, 3234, 2565, 2803, 2562, 2684, 3411, 3405, 2690, 3694, 446, 3985, 3535, 4084, 3282, 988, 2107, 990, 451, 452, 453, 992, 993, 1696, 996, 3116, 1386, 999, 458, 3879, 460, 461, 462, 2339, 1003, 4425, 1645, 1004, 466, 2328, 468, 1231, 1468, 2496, 473, 474, 2108, 1273, 1007, 1744, 1659, 478, 1009, 1012, 4313, 482, 1499, 484, 2513, 2205, 4592, 1458, 4274, 2122, 4905, 4205, 2000, 488, 1894, 3917, 3616, 4311, 4085, 4401, 1310, 3783, 1538, 3870, 1948, 3699, 1735, 3792, 2202, 4090, 2378, 2012, 3642, 1020, 2302, 4665, 4781, 500, 1022, 1023, 4686, 505, 507, 508, 509, 1025, 1732, 1816, 3636, 1028, 516, 1366, 4572, 2489, 1261, 4027, 3763, 2959, 1293, 3197, 1448, 1032, 519, 2420, 522, 3759, 1215, 4122, 1730, 524, 526, 3966, 1035, 527, 528, 529, 530, 531, 1036, 532, 533, 536, 537, 3767, 1929, 1041, 3683, 1043, 4906, 542, 1338, 1796, 4331, 4332, 4466, 3803, 3087, 3505, 1449, 1044, 4414, 3453, 3254, 544, 546, 1045, 547, 4240, 3580, 4680, 549, 550, 2598, 551, 3819, 1259, 4062, 554, 1049, 1050, 1433, 555, 1052, 1053, 3738, 3857, 2089, 1056, 2193, 1257, 1238, 3662, 1059, 1060, 4203, 3981, 1303, 4116, 567, 569, 3620, 4002, 3679, 3707, 4497, 4438, 1264, 3939, 4170, 4056, 1271, 1451, 1654, 3934, 4080, 4352, 574, 3739, 1565, 1419, 3950, 3716, 1465, 3726, 3881, 4134, 4347, 4012, 3938, 4066, 4164, 3630, 4229, 4292, 4215, 1729, 4223, 4455, 4512, 3633, 4070, 4104, 4162, 4443, 3761, 1587, 3592, 1891, 4469, 1963, 4129, 1360, 3949, 1861, 4120, 1292, 4293, 3839, 3983, 3928, 4295, 3715, 3855, 3609, 1990, 4007, 3722, 1624, 3924, 3952, 580, 2003, 1343, 582, 4114, 1818, 1704, 4448, 584, 1078, 1786, 585, 1832, 586, 2891, 587, 4936, 1082, 4459, 2113, 590, 2308, 593, 1858, 597, 2738, 4150, 3260, 3481, 4439, 1268, 4050, 3377, 4424, 3492, 1412, 4477, 4263, 3072, 2009, 3888, 3827, 4103, 1438, 609, 610, 611, 3701, 4389, 613, 4369, 617, 1921, 1373, 1870, 4218, 1099, 1702, 4801, 623, 3700, 1788, 625, 4878, 1423, 1339, 3875, 628, 2557, 2906, 2029, 4876, 1103, 631, 4479, 3766, 632, 1104, 1105, 634, 4651, 1108, 1813, 636, 638, 641, 4581, 1112, 2164, 3735, 3390, 2558, 3314, 3086, 3029, 2956, 3517, 2651, 2914, 2680, 2632, 3121, 3100, 3528, 3526, 3084, 4220, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 4398, 1269, 3624, 4481, 1432, 3295, 1568, 1766, 2643, 2739, 4760, 1115, 3920, 644, 645, 646, 4279, 3762, 1637, 1509, 2200, 1117, 4780, 2965, 2695, 650, 651, 653, 654, 655, 658, 4035, 2910, 3391, 2808, 3676, 2852, 3284, 3537, 1319, 4467, 1802, 1227, 660, 4812, 2364, 1124, 1125, 665, 4917, 1127, 1129, 1132, 1133, 2236, 673, 4557, 1136, 1139, 678, 4737, 4531, 679, 680, 1144, 681, 4525, 2415, 685, 2445, 3822, 690, 691, 1601, 4621, 3905, 695, 1149, 696, 2027, 2160, 1795, 1372, 4115, 2194, 1920, 4031, 1393, 1277, 1400, 4402, 4157, 4028, 704, 1902, 1510, 4182, 1153, 4476, 2053, 710, 1375, 715, 4096, 717, 1155, 719, 1311, 722, 4217, 1156, 723, 1157, 1627, 4566, 1158, 724, 725, 1953, 1201, 3787, 3747, 1846, 3815, 1822, 1537, 1318, 4294, 733, 734, 3439, 2833, 4505, 4267, 3697, 4112, 2967, 3651, 1805, 1163, 3859, 2275, 737, 1167, 1745, 3807, or 4307.

In some aspects, X⁶ of SEQ ID NO: 5014 is K or R. In some aspects, the 581 to 589 region of the VP capsid polypeptide has a sequence of any one of SEQ ID NOs: 738, 4177, 1289, 740, 14, 743, 744, 17, 3477, 1548, 3051, 2982, 2877, 746, 19, 3036, 3426, 3073, 3169, 747, 3362, 2784, 21, 3192, 22, 1638, 748, 24, 749, 27, 1703, 1836, 28, 3660, 29, 750, 1640, 33, 35, 36, 1653, 1205, 3795, 753, 4288, 4219, 2770, 2962, 4241, 4107, 2718, 3317, 4391, 3263, 2553, 3947, 2710, 2608, 3467, 2796, 2961, 2529, 3320, 2674, 2635, 3446, 3053, 3250, 3688, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 1467, 3327, 3178, 3006, 3252, 2991, 3470, 2888, 2572, 3418, 2642, 2519, 3363, 40, 2633, 44, 45, 756, 47, 48, 1952, 3122, 2551, 3718, 3484, 3577, 1308, 3960, 1378, 2994, 3080, 2589, 3681, 51, 760, 762, 3925, 3654, 763, 59, 60, 4086, 1740, 1660, 62, 1966, 63, 2438, 768, 66, 67, 68, 3816, 2599, 2706, 72, 73, 74, 3865, 1549, 1361, 3882, 773, 3713, 1942, 1312, 4501, 76, 1413, 775, 1560, 4289, 3757, 3593, 3908, 4397, 4415, 3723, 3754, 4360, 3945, 2854, 3523, 3669, 1356, 4065, 1294, 1484, 85, 86, 3891, 4456, 4040, 4496, 1982, 2001, 1478, 1189, 1490, 1885, 1926, 89, 780, 1180, 92, 781, 782, 783, 786, 94, 95, 788, 1831, 1867, 96, 4458, 97, 1970, 1936, 1590, 98, 99, 789, 790, 1298, 1688, 791, 792, 2007, 102, 103, 104, 105, 107, 108, 1579, 1906, 111, 798, 112, 799, 800, 1444, 801, 114, 115, 1344, 116, 802, 117, 118, 119, 121, 803, 1463, 1760, 806, 126, 128, 807, 129, 131, 808, 133, 1978, 811, 812, 135, 136, 137, 138, 814, 140, 1326, 141, 142, 143, 2344, 816, 1634, 1302, 817, 818, 147, 148, 149, 150, 151, 154, 155, 819, 820, 156, 157, 158, 161, 162, 165, 822, 168, 169, 170, 823, 172, 1651, 173, 1483, 174, 825, 176, 178, 180, 827, 181, 828, 4911, 184, 186, 188, 189, 830, 192, 193, 194, 196, 1391, 198, 199, 200, 201, 202, 203, 205, 832, 206, 834, 207, 208, 209, 210, 835, 836, 1334, 3901, 3312, 2649, 1450, 4386, 3028, 1446, 1522, 3490, 217, 219, 1612, 220, 221, 222, 839, 223, 840, 224, 225, 228, 229, 230, 231, 842, 232, 233, 234, 238, 847, 239, 850, 851, 853, 1536, 240, 241, 242, 243, 244, 245, 247, 250, 251, 254, 255, 857, 256, 258, 259, 859, 260, 861, 862, 864, 263, 2318, 865, 2326, 264, 1241, 266, 1477, 867, 268, 868, 269, 270, 869, 870, 271, 871, 872, 272, 873, 1252, 1591, 875, 274, 275, 276, 876, 877, 277, 278, 280, 1892, 1539, 881, 3601, 3877, 1460, 282, 1799, 284, 1626, 286, 1374, 1620, 1290, 1354, 887, 1956, 889, 292, 4324, 4138, 891, 296, 3903, 297, 3829, 3809, 4271, 3844, 1436, 300, 1617, 1961, 302, 304, 305, 2147, 1589, 4370, 307, 3646, 4059, 1431, 1331, 312, 1779, 313, 896, 1558, 1552, 318, 319, 322, 1958, 323, 324, 901, 1491, 328, 329, 4484, 907, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3414, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 4248, 1557, 3804, 1602, 4188, 1330, 4349, 3337, 3533, 2629, 3038, 2539, 3567, 3291, 3348, 3115, 3359, 2993, 3079, 3506, 2527, 2963, 2938, 2516, 3113, 2639, 3143, 3044, 2747, 3154, 3280, 3039, 2743, 2902, 3056, 2554, 2522, 3103, 3210, 3434, 2656, 4251, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 2869, 2610, 3298, 2775, 3108, 2603, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 2584, 2712, 3213, 3451, 2634, 3150, 2810, 3303, 2573, 3460, 2615, 3394, 2828, 3329, 4377, 4499, 3555, 2974, 3629, 3578, 3885, 2817, 3248, 2954, 3334, 3571, 3345, 3551, 3384, 3422, 2530, 2973, 4418, 4081, 3951, 1764, 1671, 1843, 3449, 3185, 3399, 2786, 3055, 3472, 3397, 2601, 3564, 3277, 3548, 3179, 3407, 3841, 3347, 3142, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3120, 3286, 2843, 3465, 3946, 2682, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 4269, 3487, 3489, 3019, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1464, 1886, 2666, 3246, 3130, 3560, 3236, 2730, 3572, 3689, 2968, 1457, 3450, 2577, 2567, 2532, 3415, 2934, 2984, 2568, 4147, 3002, 2986, 910, 2979, 2542, 1762, 4472, 1529, 4057, 1976, 4507, 1583, 1711, 2013, 1594, 4440, 344, 920, 921, 4498, 3728, 3637, 3570, 3429, 4091, 2870, 2995, 4338, 3205, 2880, 3261, 3968, 4291, 4204, 4071, 4034, 1742, 2820, 3912, 3287, 1218, 1190, 4145, 354, 1782, 927, 3610, 356, 4224, 1918, 1959, 1840, 3736, 1770, 929, 930, 931, 4155, 360, 361, 362, 364, 1279, 365, 1543, 366, 367, 4049, 1633, 1722, 369, 1495, 1566, 373, 1211, 1726, 1442, 1748, 4142, 1434, 1358, 1191, 3732, 4110, 4039, 4506, 4156, 4036, 4030, 4016, 3899, 1247, 1855, 3919, 1244, 4140, 3930, 4117, 4189, 4001, 3643, 3619, 1240, 1575, 3871, 4135, 4408, 3604, 1274, 4149, 3741, 1938, 3709, 3622, 3834, 1983, 1743, 3979, 4461, 4113, 3817, 4232, 4214, 3902, 1219, 1486, 1512, 1516, 4047, 4082, 4094, 1574, 1248, 4078, 1872, 1426, 3687, 4318, 4243, 3866, 3704, 4172, 1856, 3324, 2769, 2678, 1561, 3296, 377, 1395, 1584, 4093, 4453, 4210, 4009, 4264, 4339, 1738, 4474, 4013, 4266, 1771, 1759, 1922, 3860, 3832, 1349, 1381, 3663, 1185, 3932, 3858, 1198, 4192, 3611, 1919, 3790, 3712, 4095, 1887, 4468, 1720, 4247, 934, 1790, 2002, 1657, 1336, 3894, 3576, 4314, 3695, 1850, 4362, 1815, 3638, 4454, 1661, 4504, 1359, 3650, 1305, 1648, 3831, 4435, 4451, 1993, 3849, 1718, 4068, 1835, 1425, 1428, 4380, 1658, 1452, 4024, 4072, 3791, 4491, 4323, 4014, 1304, 1511, 1981, 1676, 4422, 1283, 3861, 3644, 4259, 1418, 1176, 4216, 1421, 3992, 4423, 4253, 3748, 4119, 1297, 1954, 4450, 3711, 4344, 1578, 4384, 3892, 3674, 3595, 4405, 3973, 4109, 4159, 3744, 4387, 4275, 4492, 4365, 4118, 4278, 3751, 3678, 4353, 3175, 2970, 1228, 3534, 3437, 3406, 3641, 4470, 3941, 4348, 3587, 3818, 4350, 3821, 3929, 3824, 4465, 1398, 3828, 1284, 3773, 4209, 3014, 3884, 2783, 4038, 3545, 2916, 3112, 2834, 3880, 4399, 4048, 3752, 4207, 1235, 3943, 4355, 4087, 4100, 4163, 4486, 4178, 1643, 4152, 4445, 4303, 4284, 1213, 4382, 1399, 3714, 4487, 3615, 4018, 4420, 4121, 3724, 4282, 4310, 4488, 4127, 3670, 4052, 3627, 3777, 1371, 3685, 4258, 3812, 1581, 4124, 4508, 4500, 3652, 3972, 1528, 3717, 2740, 3125, 3546, 3842, 4404, 3690, 3962, 4136, 3589, 3907, 4153, 2707, 4180, 3923, 4444, 4361, 4394, 3758, 4510, 3742, 1710, 1407, 4187, 1193, 1188, 1196, 1724, 1717, 1200, 1997, 1701, 4280, 4006, 1984, 3833, 2561, 4442, 3900, 4298, 1761, 4388, 1597, 4317, 4249, 1341, 1203, 1680, 4441, 3628, 3725, 1945, 937, 4206, 1588, 1439, 3794, 4447, 3776, 3658, 4064, 4011, 4272, 4503, 1324, 3594, 3680, 1300, 1750, 3913, 3774, 1877, 1987, 3998, 3686, 3696, 4463, 3853, 4244, 4083, 1909, 3897, 1546, 3613, 3273, 3452, 3525, 1229, 3835, 1234, 3970, 3856, 3586, 1175, 4321, 2778, 4412, 4407, 3823, 1427, 1507, 1878, 4333, 3146, 4281, 4211, 3843, 1677, 1524, 1525, 1974, 1239, 4167, 3740, 1342, 1471, 4176, 1232, 1989, 4125, 1741, 1580, 4060, 1576, 1212, 3921, 1174, 4061, 4021, 3693, 3734, 1382, 3684, 3806, 3635, 3976, 3840, 4286, 3836, 4161, 3659, 3605, 4392, 4328, 1316, 4173, 4265, 1246, 1629, 1829, 3826, 1992, 1656, 1914, 1839, 3984, 3910, 4160, 4184, 4132, 4250, 1792, 3769, 1534, 4003, 1689, 1599, 1265, 1410, 1823, 1498, 1329, 386, 940, 1655, 3874, 4261, 3626, 3579, 1644, 3936, 4054, 4260, 4297, 4357, 4022, 4046, 1222, 1996, 3788, 3953, 4226, 4277, 4101, 3797, 4019, 3771, 3632, 1402, 4137, 3772, 3661, 4186, 4174, 1170, 3850, 1315, 1611, 1859, 4183, 1403, 4033, 3623, 3596, 1461, 1848, 1173, 1928, 1765, 3719, 3584, 4165, 4191, 3980, 4058, 4200, 4336, 3958, 1260, 4051, 3798, 4032, 1774, 4322, 3991, 4106, 3978, 3965, 4464, 3813, 4154, 1365, 3846, 4345, 4379, 3603, 3937, 3801, 1787, 1214, 4483, 4201, 1845, 1220, 4245, 1347, 3867, 1900, 3640, 3916, 4089, 3745, 3975, 4256, 4308, 4000, 3868, 4403, 3883, 4326, 4393, 3895, 4237, 3955, 4005, 4092, 4372, 4283, 3805, 4151, 3982, 3597, 3872, 3987, 4490, 3876, 3668, 4300, 1679, 1242, 3598, 3583, 3647, 4304, 3779, 1907, 4042, 3648, 3996, 3800, 4367, 4099, 1807, 3887, 4428, 1625, 1390, 1884, 1682, 4364, 4306, 3869, 1278, 942, 1505, 1709, 3600, 3914, 3948, 1865, 1716, 3961, 1506, 4221, 1376, 1683, 4276, 1721, 1632, 3607, 1455, 1913, 1497, 1317, 1951, 1778, 3768, 4225, 1668, 390, 4406, 4341, 1480, 1714, 1422, 397, 3618, 4433, 1177, 3898, 398, 950, 400, 1965, 4471, 953, 401, 403, 955, 1482, 3602, 4434, 3721, 1592, 1825, 1698, 1519, 1352, 4409, 1977, 405, 1631, 1684, 409, 3889, 2117, 411, 413, 414, 415, 961, 1905, 416, 963, 3675, 1833, 3974, 965, 970, 1897, 1272, 4133, 3830, 972, 424, 427, 1708, 4141, 3749, 1619, 975, 4044, 3590, 1944, 4126, 1923, 1706, 432, 976, 978, 4400, 1606, 980, 2604, 2895, 436, 3448, 2188, 438, 1692, 442, 3862, 4055, 4358, 2276, 985, 1664, 443, 4309, 4045, 3854, 3682, 1972, 2688, 4366, 3425, 3231, 3649, 2759, 2724, 3755, 1216, 3599, 3775, 2754, 2606, 3471, 3234, 2565, 2803, 2562, 2684, 3411, 3405, 2690, 1255, 3694, 3985, 3535, 1306, 4084, 3282, 1780, 448, 990, 991, 1485, 992, 1728, 993, 994, 1696, 997, 3116, 1869, 456, 1386, 1000, 3879, 2698, 460, 1001, 461, 1447, 1002, 1003, 463, 464, 4425, 465, 1004, 466, 469, 1231, 1468, 470, 1005, 476, 1273, 1744, 1488, 1659, 1604, 479, 1013, 1014, 481, 4313, 1499, 1016, 4073, 1777, 485, 4274, 487, 1775, 4205, 1526, 1697, 2000, 488, 1894, 1882, 1817, 1880, 490, 1018, 3917, 3616, 4311, 4473, 4085, 4401, 3783, 3765, 1538, 3870, 491, 3699, 3792, 4290, 494, 2421, 4090, 2012, 1803, 3642, 1322, 1020, 502, 1333, 504, 1890, 507, 3608, 1732, 1864, 1816, 1531, 513, 3636, 514, 1699, 1027, 1366, 4950, 4027, 3478, 2959, 1293, 3197, 1595, 3413, 518, 1032, 1033, 1675, 1797, 2315, 521, 1999, 3759, 1215, 4122, 1730, 3966, 1035, 4973, 528, 3911, 3289, 1821, 530, 532, 1874, 533, 1038, 538, 539, 3767, 1929, 3683, 1338, 1616, 1901, 1796, 1517, 4331, 4332, 4466, 2719, 4123, 4233, 1824, 3129, 3087, 3505, 4158, 4254, 3720, 4351, 4414, 3453, 3254, 1649, 544, 4240, 3580, 1046, 548, 549, 2598, 552, 553, 3819, 1259, 4062, 554, 2841, 3515, 1433, 1323, 4301, 555, 558, 3738, 1567, 559, 1054, 1270, 3857, 2175, 1257, 1238, 3662, 561, 1058, 563, 1061, 4203, 1899, 3981, 1062, 1303, 4116, 1946, 3620, 1514, 4002, 3679, 3964, 3707, 3666, 1348, 4273, 1804, 4497, 1401, 4438, 1895, 1264, 1912, 573, 3939, 4170, 4056, 1271, 1521, 1221, 1451, 3934, 4080, 4352, 1700, 3739, 1419, 1811, 3950, 1690, 3716, 1569, 1437, 1674, 4337, 1285, 4268, 1183, 3881, 3938, 4198, 4416, 4229, 4292, 4193, 4325, 4375, 4432, 4215, 3944, 4195, 4223, 1540, 4455, 4512, 1723, 3750, 4070, 4104, 4235, 1863, 4346, 1881, 1814, 4443, 3761, 1587, 3592, 576, 4469, 1275, 1950, 4129, 1296, 1551, 3733, 4111, 3993, 4108, 1287, 1713, 1556, 1736, 1995, 1705, 1861, 4120, 4417, 1785, 1236, 4293, 1827, 3839, 3983, 4395, 1957, 3928, 4295, 3715, 3855, 3609, 4076, 4175, 3927, 1757, 4299, 1773, 1364, 1462, 4007, 3729, 3665, 3722, 1691, 1712, 1624, 1751, 1394, 3924, 3952, 581, 2003, 1559, 1250, 1555, 4114, 1818, 1076, 4448, 1078, 1786, 3354, 1832, 3432, 2891, 1903, 4459, 589, 1563, 1084, 591, 592, 594, 595, 1858, 596, 1453, 2559, 2590, 2738, 4478, 4069, 1809, 1327, 4150, 3260, 3481, 3672, 3625, 4439, 3852, 3997, 3847, 4050, 3377, 4424, 4143, 4493, 3492, 1412, 4017, 4477, 1794, 602, 4263, 3072, 2009, 1754, 3888, 603, 604, 1368, 3827, 4103, 1571, 1438, 611, 1093, 612, 1094, 3701, 4389, 614, 615, 1332, 1715, 4369, 1652, 1541, 1921, 1733, 1373, 1870, 619, 4218, 1798, 1702, 1384, 3700, 1788, 1600, 624, 2473, 1423, 2771, 1339, 3875, 3070, 2015, 2557, 2795, 2906, 2103, 3299, 3118, 4576, 1103, 1614, 1947, 631, 4479, 3766, 1107, 635, 1813, 636, 2431, 637, 1109, 639, 3706, 1110, 640, 642, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 1269, 3095, 1286, 3624, 4481, 3511, 2735, 4381, 3295, 1568, 1766, 3221, 2643, 2739, 3920, 647, 1801, 4279, 3762, 1637, 1509, 648, 1117, 1119, 2965, 2695, 3293, 4722, 1121, 653, 1122, 3264, 655, 656, 657, 4035, 2910, 3391, 2808, 3099, 2913, 3676, 2852, 3284, 2731, 3537, 3994, 1319, 4467, 1623, 660, 1123, 664, 1126, 1128, 1129, 1130, 670, 1527, 671, 1772, 1137, 5005, 1933, 1731, 1793, 3342, 1140, 1141, 687, 3822, 1601, 1148, 693, 3905, 694, 1414, 1149, 697, 1795, 1372, 1695, 1866, 1151, 4115, 1719, 1920, 4999, 1544, 703, 4031, 4305, 1636, 3810, 4452, 3677, 4227, 4402, 4157, 4197, 1337, 1253, 4028, 3989, 704, 1493, 1902, 1152, 4182, 707, 708, 709, 4476, 1375, 713, 714, 1841, 4234, 1154, 1314, 4096, 718, 719, 720, 2063, 1311, 721, 4217, 1627, 1158, 1953, 1201, 3787, 729, 3747, 730, 1846, 3815, 1964, 1822, 1262, 4480, 1537, 1291, 1318, 4294, 1582, 3439, 2833, 4287, 4509, 4505, 4267, 3697, 3272, 4112, 2967, 3651, 1805, 3705, 1195, 3859, 1164, 1165, 2351, 2423, 1745, 1973, 3807, 4307, 1169, 1454, or 1781.

In some aspects, X⁷ of SEQ ID NO: 5014 is R. In some aspects, the 581 to 589 region of the VP capsid polypeptide has a sequence of any one of SEQ ID NOs: 740, 15, 742, 743, 17, 22, 24, 1836, 32, 36, 2450, 39, 752, 753, 1998, 3059, 2519, 757, 48, 1952, 50, 52, 760, 2389, 54, 2346, 58, 4086, 1660, 1966, 768, 2402, 772, 71, 72, 2453, 3882, 775, 1806, 3669, 1356, 2132, 81, 84, 85, 87, 4040, 88, 1885, 782, 783, 784, 785, 94, 788, 2173, 98, 2007, 102, 113, 799, 4848, 1444, 4821, 2329, 115, 119, 2290, 123, 806, 128, 134, 812, 145, 817, 154, 2149, 821, 167, 171, 4596, 183, 830, 191, 194, 2071, 195, 4920, 831, 2223, 207, 3901, 211, 212, 213, 215, 224, 4782, 845, 846, 849, 850, 854, 247, 248, 2245, 255, 261, 264, 265, 1477, 270, 874, 876, 279, 882, 1930, 285, 286, 886, 2119, 2468, 887, 888, 4138, 297, 3844, 302, 893, 307, 308, 2155, 1431, 2312, 900, 1635, 906, 2799, 2128, 2918, 1363, 4251, 2622, 3333, 3474, 3614, 2732, 3166, 3151, 3088, 2883, 3318, 2645, 2733, 3213, 3782, 1844, 2523, 2520, 2997, 2681, 3035, 3279, 2764, 3480, 2787, 1808, 3101, 3689, 4147, 1762, 1976, 340, 1594, 2439, 348, 4498, 1518, 1782, 926, 357, 359, 932, 363, 369, 1873, 3780, 4140, 4001, 3709, 3834, 1752, 4113, 3817, 4232, 1340, 1489, 4214, 3902, 3820, 4330, 1872, 3687, 4318, 375, 4210, 3860, 4212, 3695, 1851, 4504, 1910, 1428, 4323, 4422, 4259, 1176, 3711, 4365, 4383, 3969, 4128, 3802, 3863, 3821, 4171, 3824, 4462, 3760, 3753, 2783, 3880, 3943, 4163, 4178, 4199, 3724, 3670, 4482, 3692, 4430, 4316, 3645, 3690, 3589, 3730, 4426, 3851, 4429, 1335, 1717, 1997, 1975, 4280, 4006, 3900, 4298, 4388, 1341, 1350, 1909, 4412, 4407, 4281, 4167, 1893, 3585, 4015, 1747, 4286, 3826, 1473, 1914, 3984, 4495, 4184, 3977, 1707, 4074, 4003, 3915, 1410, 1498, 940, 4131, 4357, 1925, 1466, 1611, 1530, 4168, 4245, 4075, 3987, 1679, 4306, 3869, 3768, 393, 2265, 3618, 4433, 947, 399, 401, 402, 4139, 1352, 406, 415, 418, 420, 4919, 431, 4044, 976, 433, 434, 435, 442, 3873, 3411, 3985, 987, 1485, 2262, 457, 458, 4425, 466, 467, 468, 1468, 474, 1008, 1012, 1016, 4518, 1697, 490, 3616, 2247, 2302, 499, 500, 501, 509, 2110, 512, 1797, 520, 525, 3966, 1038, 537, 1041, 3683, 2165, 542, 4331, 3129, 1044, 4351, 3453, 1047, 1323, 4301, 557, 3738, 559, 1270, 3857, 1056, 2186, 1057, 1059, 565, 566, 3981, 567, 3620, 4002, 3666, 3939, 1221, 1397, 1437, 4337, 4268, 3938, 4292, 4215, 3750, 3633, 4070, 3592, 1827, 3839, 4395, 3855, 3665, 1624, 2395, 1818, 586, 587, 1903, 1080, 1081, 1082, 589, 1084, 1085, 594, 596, 4439, 3997, 1962, 1087, 4777, 606, 607, 610, 1094, 4389, 1603, 1095, 616, 618, 622, 1104, 4536, 3706, 2481, 3100, 3200, 3621, 4179, 1432, 4381, 643, 1115, 644, 2307, 2477, 652, 657, 658, 3994, 4467, 661, 663, 665, 4600, 1137, 1933, 4840, 1140, 678, 680, 683, 686, 692, 693, 696, 2081, 2280, 698, 1400, 4197, 1532, 1854, 706, 711, 712, 4570, 714, 1841, 1314, 4096, 2249, 726, 1846, 2385, 1318, 733, 3651, 1165, 4839, 4968, 4684, or 1454.

In some aspects, (a) X³ of SEQ ID NO: 5014 is a basic amino acid residue, (b) X⁶ of SEQ ID NO: 5014 is a basic amino acid residue, or (c) X³ of SEQ ID NO: 5014 is a basic amino acid residue and X⁶ of SEQ ID NO: 5014 is a basic amino acid residue. In some aspects, the 581 to 589 region of the VP capsid polypeptide has a sequence of any one of SEQ ID NOs: 739, 4177, 1289, 740, 741, 18, 745, 3477, 1548, 3051, 2982, 2877, 3036, 3426, 3073, 3169, 747, 3362, 2784, 21, 3192, 23, 1638, 748, 1703, 1836, 3660, 29, 750, 1640, 33, 34, 35, 36, 37, 38, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 3327, 3178, 3006, 3252, 2991, 3470, 2888, 2572, 3418, 2642, 2519, 3363, 40, 2633, 41, 42, 43, 755, 45, 756, 46, 48, 1952, 758, 49, 3122, 2551, 3718, 3484, 3577, 759, 1230, 3960, 1378, 2994, 3080, 2589, 3681, 52, 760, 53, 762, 4659, 54, 3925, 55, 3654, 56, 60, 61, 4086, 1740, 1660, 765, 766, 62, 1966, 63, 64, 768, 771, 65, 66, 67, 68, 3816, 2599, 2706, 2399, 74, 3865, 1549, 1361, 2453, 3882, 3713, 1942, 75, 1312, 4501, 76, 77, 78, 1560, 4289, 3757, 79, 3593, 4043, 1806, 3908, 4397, 1615, 4415, 3723, 2006, 3754, 1470, 4360, 3945, 2854, 3523, 3669, 82, 4065, 83, 1294, 1484, 3891, 4456, 4040, 4496, 1982, 2001, 1478, 1189, 88, 1490, 1885, 1926, 89, 90, 91, 779, 1180, 92, 785, 786, 94, 95, 1831, 1867, 4458, 97, 1970, 1936, 1590, 99, 100, 789, 790, 1298, 101, 1688, 791, 792, 2007, 103, 794, 795, 104, 105, 106, 1849, 108, 4736, 1579, 110, 1906, 113, 1444, 1344, 116, 802, 117, 118, 2158, 121, 803, 804, 1463, 805, 122, 2058, 124, 125, 127, 4994, 807, 132, 809, 810, 133, 1978, 812, 135, 136, 137, 138, 813, 4673, 140, 815, 1326, 141, 142, 143, 144, 145, 4816, 1634, 1302, 817, 146, 818, 147, 148, 149, 151, 152, 153, 154, 155, 819, 820, 157, 821, 159, 160, 163, 164, 165, 166, 822, 167, 168, 169, 170, 171, 823, 824, 1651, 173, 1483, 174, 175, 825, 176, 177, 180, 826, 827, 181, 828, 182, 183, 4641, 829, 184, 185, 186, 187, 188, 189, 830, 191, 192, 193, 194, 195, 196, 1391, 197, 2223, 200, 201, 202, 203, 204, 205, 832, 833, 206, 834, 835, 836, 1334, 837, 3901, 3312, 2649, 1301, 211, 212, 1450, 4386, 3028, 213, 1446, 214, 215, 1896, 1522, 3490, 838, 2241, 217, 218, 219, 1612, 220, 221, 222, 839, 223, 840, 224, 226, 227, 230, 231, 842, 232, 233, 235, 844, 236, 237, 2150, 851, 852, 853, 1536, 240, 854, 242, 243, 244, 245, 246, 247, 855, 4634, 249, 250, 251, 252, 253, 254, 255, 857, 256, 257, 258, 259, 858, 859, 260, 261, 860, 861, 862, 262, 2326, 264, 1241, 265, 266, 866, 1477, 267, 867, 869, 870, 271, 871, 872, 272, 874, 1252, 273, 1591, 275, 1980, 276, 876, 877, 277, 278, 279, 1892, 879, 1539, 880, 1295, 3601, 3877, 1930, 1420, 1678, 1460, 2306, 282, 1799, 283, 1626, 1374, 287, 1620, 1290, 1354, 888, 291, 1956, 889, 292, 4324, 293, 4138, 891, 2224, 3903, 297, 3829, 3809, 4271, 3844, 1436, 1515, 1617, 1961, 304, 306, 1589, 4370, 308, 309, 894, 3646, 4059, 1431, 1331, 4948, 312, 895, 1779, 313, 314, 315, 316, 896, 1558, 1552, 897, 898, 321, 322, 899, 1958, 2134, 324, 901, 325, 902, 326, 1491, 329, 330, 905, 4484, 1756, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 1888, 4248, 1557, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 3435, 3482, 3020, 2748, 2646, 2787, 2760, 2563, 3085, 3133, 3158, 3170, 2703, 3494, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 2979, 2542, 2492, 1762, 4472, 1472, 1529, 4057, 1976, 4507, 1583, 338, 911, 1711, 340, 912, 2013, 341, 1594, 4440, 917, 344, 918, 919, 921, 922, 347, 349, 351, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 4145, 354, 925, 1782, 926, 927, 3610, 357, 358, 359, 4224, 1918, 1959, 3736, 1770, 931, 4155, 364, 1279, 365, 1543, 2472, 367, 4049, 1633, 1722, 368, 369, 1495, 1566, 370, 1211, 1873, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 3741, 1938, 3709, 1727, 3893, 1280, 3622, 1784, 3834, 1752, 1983, 1743, 3979, 4461, 4113, 3817, 4232, 1340, 1489, 4214, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 377, 1395, 4093, 4453, 3940, 3778, 4374, 4210, 4009, 4264, 4339, 4368, 1738, 378, 1610, 4194, 4474, 4013, 4266, 1771, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 2002, 1657, 1663, 1336, 3894, 1554, 1494, 1405, 3576, 4314, 3695, 1851, 4362, 1815, 3845, 4511, 3656, 3638, 1406, 1596, 4454, 1443, 1661, 1359, 1734, 3650, 1910, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1618, 1871, 1256, 3748, 4119, 1297, 1586, 1251, 1299, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1680, 4441, 3628, 3725, 1945, 937, 4206, 1588, 1439, 3794, 4447, 1838, 3776, 3658, 4064, 1377, 4011, 4272, 4503, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 3613, 3273, 3452, 3525, 1229, 4105, 3835, 383, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 4125, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 1520, 1767, 1466, 1194, 4137, 1908, 1927, 1758, 3772, 3661, 4186, 4174, 1170, 3850, 1315, 1611, 1530, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 1716, 3961, 1506, 4221, 1376, 1683, 4276, 1445, 1721, 1632, 3607, 1328, 1455, 1913, 1497, 1317, 1951, 1778, 1379, 3768, 4225, 1668, 390, 391, 4406, 4341, 1480, 395, 1714, 1422, 396, 2265, 397, 3618, 4433, 1177, 3898, 398, 949, 950, 399, 951, 400, 1965, 2345, 4471, 952, 402, 2475, 403, 404, 4139, 1482, 3602, 4434, 3721, 1592, 1519, 1352, 4202, 4409, 1977, 1917, 407, 408, 1631, 1684, 409, 3889, 958, 959, 412, 960, 414, 415, 961, 1905, 416, 963, 417, 3675, 1833, 3974, 420, 421, 965, 966, 969, 970, 422, 1897, 4754, 423, 4133, 3830, 972, 424, 973, 427, 1708, 4141, 3749, 431, 1619, 975, 4044, 3590, 1944, 4126, 1923, 1706, 4400, 434, 1606, 979, 2604, 2895, 436, 3448, 1692, 983, 3862, 4055, 4358, 984, 985, 1664, 4045, 3854, 3682, 2491, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 3535, 1306, 4084, 3282, 1780, 987, 988, 450, 990, 451, 452, 991, 992, 1728, 994, 995, 454, 1696, 998, 3116, 1869, 456, 1386, 999, 3879, 2698, 1001, 461, 1447, 462, 464, 4425, 1645, 1231, 1468, 472, 473, 4725, 476, 1273, 1007, 1744, 1488, 1659, 477, 1604, 481, 4313, 482, 483, 1499, 484, 2513, 1016, 4073, 1777, 1458, 4274, 4539, 4528, 487, 1775, 4205, 1526, 1697, 1017, 2000, 489, 1882, 1817, 1880, 490, 1018, 3917, 3616, 4311, 1800, 4473, 4085, 4401, 1310, 3783, 3765, 491, 1948, 3699, 1735, 3792, 4290, 495, 4090, 2247, 496, 1019, 2012, 1803, 3642, 1322, 498, 1021, 501, 1022, 502, 1333, 503, 1023, 1890, 1025, 510, 1026, 3608, 1732, 511, 2397, 1864, 1816, 1531, 3636, 1699, 1027, 1029, 1366, 2112, 2124, 2376, 1261, 4027, 3478, 3763, 2959, 517, 3197, 1595, 3413, 1448, 518, 2057, 1675, 1797, 2148, 2420, 520, 2315, 523, 1999, 3759, 1215, 4122, 1730, 524, 526, 3966, 3911, 529, 3289, 1821, 530, 531, 1874, 1038, 536, 537, 3767, 1929, 1041, 1042, 3683, 542, 1338, 1517, 4331, 4332, 4466, 2719, 3803, 4123, 4233, 1824, 3129, 3087, 3505, 4158, 4254, 1449, 3720, 4351, 4414, 1673, 3453, 3254, 1649, 544, 545, 546, 4240, 3580, 1046, 1047, 550, 2598, 551, 552, 3819, 1259, 4062, 2841, 1050, 3515, 4301, 557, 1052, 1053, 3738, 1567, 1270, 3857, 1055, 2198, 1257, 1238, 3662, 561, 562, 1058, 566, 1060, 1061, 2231, 4203, 1899, 3981, 1062, 1303, 4116, 567, 1946, 3620, 1514, 4002, 3679, 3964, 3707, 3666, 1348, 4273, 1804, 4497, 1401, 4438, 1895, 1264, 572, 1912, 3939, 4170, 4056, 1271, 1065, 1521, 1221, 1451, 1654, 3934, 4080, 4352, 1700, 3739, 1565, 1419, 1811, 3950, 1690, 1397, 3716, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 1723, 1070, 3750, 3633, 4070, 4104, 4162, 4235, 1863, 4346, 1881, 1814, 1572, 4443, 3761, 1346, 1587, 1072, 3592, 576, 1891, 4469, 1275, 1950, 1963, 4129, 1296, 1551, 3733, 4111, 3993, 4108, 3949, 1287, 1713, 1736, 1995, 1435, 1424, 1830, 1705, 1861, 1197, 1254, 4120, 1545, 4417, 1469, 1785, 1236, 4293, 3839, 3983, 4395, 1957, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1624, 1751, 1394, 3924, 3952, 580, 581, 2003, 1343, 1559, 1250, 1555, 4114, 1818, 1075, 1076, 1077, 1704, 4448, 1078, 1079, 1786, 585, 3354, 1832, 3432, 2891, 2505, 1903, 1081, 1082, 4459, 1083, 1563, 590, 591, 1085, 594, 597, 598, 1453, 2559, 2590, 2738, 4478, 4069, 1809, 1327, 4150, 3260, 3481, 3672, 3625, 2008, 4439, 3852, 3997, 1411, 599, 3847, 1268, 4050, 3377, 4424, 1496, 4143, 4493, 3492, 1962, 600, 601, 4017, 4477, 1794, 4263, 3072, 2009, 1754, 1087, 1088, 3888, 2324, 605, 1368, 3827, 4103, 606, 4549, 1090, 1571, 1091, 1438, 1092, 1093, 612, 1094, 3701, 4389, 613, 1603, 616, 1332, 1715, 4369, 1652, 1096, 617, 1541, 618, 1921, 1733, 1373, 1870, 619, 4218, 1798, 1099, 1100, 1702, 2239, 1101, 1667, 1384, 623, 3700, 1788, 1600, 624, 626, 2771, 627, 1339, 3875, 628, 629, 3070, 4818, 4860, 2557, 2795, 2906, 3118, 4657, 4646, 4875, 4627, 4747, 1614, 1947, 4618, 4479, 3766, 632, 1104, 1105, 633, 635, 1813, 636, 2213, 3706, 1110, 640, 1111, 641, 642, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 3095, 1286, 3624, 4481, 1432, 3511, 2735, 4381, 3295, 1585, 1114, 1766, 3221, 2643, 2739, 3920, 644, 1116, 645, 646, 2307, 647, 1801, 4279, 3762, 1637, 1509, 1119, 2965, 2369, 2695, 3293, 650, 651, 1120, 652, 1121, 4909, 1122, 3264, 655, 658, 659, 4035, 2910, 3391, 2808, 3099, 2913, 3676, 2852, 3284, 2731, 1609, 3994, 1319, 4467, 1802, 1227, 1623, 661, 2364, 662, 663, 664, 1124, 1125, 1127, 1128, 1129, 668, 669, 1130, 670, 1131, 1527, 1132, 671, 1772, 1135, 672, 5005, 2208, 4952, 4945, 1933, 1731, 1793, 675, 676, 3342, 1138, 1139, 677, 679, 1142, 1143, 681, 1145, 682, 685, 688, 3822, 689, 690, 691, 1147, 1601, 3905, 694, 695, 1414, 4793, 4575, 1150, 1795, 1372, 1695, 1866, 4115, 1719, 698, 2217, 1920, 699, 700, 1544, 702, 703, 4031, 4305, 1636, 1393, 2010, 3810, 4452, 3677, 4227, 1277, 1400, 4402, 4157, 4197, 1337, 1253, 1532, 4028, 3989, 1493, 1854, 1902, 1510, 2340, 4182, 706, 707, 708, 4476, 1245, 714, 1841, 4234, 1314, 4096, 717, 1155, 2249, 4217, 1156, 723, 1157, 1627, 725, 1159, 1953, 1160, 1201, 727, 3787, 4568, 3747, 730, 1846, 3815, 2005, 1964, 1822, 1262, 4480, 1537, 1291, 1318, 4294, 1582, 734, 3439, 2833, 4287, 4509, 4505, 4267, 3697, 3272, 4112, 2967, 3651, 1628, 3705, 1195, 3859, 1164, 2394, 1165, 1166, 1745, 1973, 1168, 3807, 4307, 2303, 1454, or 1781. In some aspects, the 581 to 589 region of the VP capsid polypeptide has a sequence of any one of SEQ ID NOs: 4884, 738, 4177, 1289, 740, 14, 743, 744, 17, 3477, 1548, 3051, 2982, 2877, 746, 19, 3036, 3426, 3073, 3169, 747, 3362, 2784, 4889, 21, 3192, 4942, 22, 1638, 748, 24, 749, 27, 1703, 1836, 28, 3660, 29, 750, 1640, 33, 35, 36, 2450, 1653, 1205, 3795, 753, 4288, 4219, 2770, 2962, 4241, 4107, 2718, 3317, 4391, 3263, 2553, 3947, 2710, 2608, 3467, 2796, 2961, 2529, 3320, 2674, 2635, 3446, 3053, 3250, 3688, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 1467, 3327, 3178, 3006, 3252, 2991, 3470, 2888, 2572, 3418, 2642, 2519, 3363, 40, 2633, 44, 45, 756, 47, 48, 1952, 3122, 2551, 3718, 3484, 3577, 759, 50, 1308, 3960, 1378, 2994, 3080, 2589, 3681, 51, 760, 2389, 762, 3925, 3654, 763, 4626, 59, 60, 4086, 1740, 1660, 62, 1966, 63, 2438, 768, 66, 67, 2197, 68, 3816, 2599, 2706, 72, 73, 74, 3865, 1549, 1361, 2453, 3882, 773, 3713, 1942, 1312, 4501, 76, 1413, 775, 1560, 4289, 3757, 3593, 3908, 4397, 4415, 3723, 3754, 4360, 3945, 2854, 3523, 3669, 1356, 2132, 4065, 1294, 1484, 85, 86, 3891, 4456, 4040, 4496, 1982, 2001, 1478, 1189, 1490, 1885, 1926, 89, 780, 1180, 92, 781, 782, 783, 2356, 786, 94, 95, 788, 1831, 1867, 96, 4458, 97, 1970, 4656, 1936, 1590, 98, 99, 789, 790, 1298, 101, 1688, 791, 792, 2007, 102, 103, 104, 105, 107, 108, 1579, 110, 1906, 111, 798, 112, 799, 800, 2461, 1444, 801, 114, 115, 1344, 116, 802, 117, 118, 119, 121, 803, 1463, 1760, 124, 806, 126, 128, 807, 129, 131, 808, 2281, 133, 1978, 811, 812, 135, 136, 137, 138, 814, 140, 1326, 141, 142, 143, 2344, 816, 1634, 1302, 817, 818, 147, 148, 149, 150, 151, 154, 155, 819, 820, 156, 157, 2295, 158, 161, 162, 165, 822, 168, 169, 170, 823, 172, 1651, 173, 1483, 174, 825, 176, 178, 180, 827, 181, 828, 4911, 184, 185, 186, 188, 189, 830, 192, 193, 194, 196, 1391, 197, 198, 199, 200, 201, 202, 203, 4775, 205, 832, 206, 834, 207, 208, 209, 210, 835, 836, 1334, 3901, 3312, 2649, 1450, 4386, 3028, 1446, 2206, 215, 1522, 3490, 217, 219, 1612, 220, 221, 222, 839, 223, 840, 224, 225, 4751, 228, 229, 230, 231, 2448, 842, 232, 233, 4782, 234, 235, 238, 847, 239, 850, 851, 853, 1536, 240, 241, 242, 243, 244, 245, 246, 247, 4923, 4696, 250, 251, 254, 255, 857, 256, 258, 259, 859, 260, 861, 862, 4966, 864, 263, 4970, 2318, 865, 2326, 264, 1241, 266, 1477, 867, 268, 868, 269, 270, 869, 870, 271, 871, 872, 272, 873, 4655, 4972, 1252, 1591, 875, 274, 275, 1980, 276, 876, 877, 2066, 277, 278, 280, 2323, 1892, 1539, 2214, 881, 882, 3601, 3877, 1930, 1460, 282, 1799, 284, 4553, 4772, 1626, 286, 1374, 1620, 1290, 1354, 887, 1956, 889, 4765, 292, 4324, 4138, 891, 296, 3903, 297, 3829, 3809, 4271, 3844, 1436, 300, 1617, 1961, 5010, 302, 304, 305, 2147, 1589, 4370, 307, 3646, 4059, 1431, 1331, 312, 1779, 313, 896, 1558, 1552, 318, 319, 322, 1958, 2454, 323, 324, 901, 1491, 328, 329, 330, 4820, 4484, 1756, 907, 4584, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3414, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 4248, 1557, 3804, 1602, 4188, 1330, 4349, 3337, 3533, 2629, 3038, 2539, 3567, 3291, 3348, 3115, 3359, 2993, 3079, 3506, 2527, 2963, 2938, 2516, 3113, 2639, 3143, 3044, 2747, 3154, 3280, 3039, 2743, 2902, 3056, 2554, 2522, 3103, 3210, 3434, 2656, 4251, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 2869, 2610, 3298, 2775, 3108, 2603, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3629, 3578, 3885, 2817, 3248, 2954, 3334, 3571, 3345, 3551, 3384, 3422, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3120, 3286, 2843, 3465, 3946, 4222, 2682, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 4269, 3487, 3489, 3019, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 3689, 2968, 1457, 3450, 2577, 2567, 2532, 3415, 2934, 2984, 2568, 4147, 3002, 2986, 910, 2979, 2542, 1762, 4472, 1529, 4057, 1976, 4507, 1583, 1711, 2013, 1594, 4440, 343, 344, 920, 921, 348, 4498, 3728, 3637, 3570, 3429, 4091, 2870, 2995, 4338, 3205, 2880, 3261, 3968, 4291, 4204, 4071, 4034, 1742, 2820, 3912, 3287, 1218, 1190, 4145, 354, 1782, 927, 3610, 356, 4224, 1918, 1959, 1840, 3736, 1770, 929, 930, 931, 4155, 360, 361, 362, 364, 1279, 365, 1543, 366, 367, 4049, 1633, 1722, 369, 1495, 1566, 373, 1211, 2494, 1726, 1442, 1748, 4142, 1434, 1358, 1191, 3732, 4110, 4039, 4411, 4506, 4156, 4036, 4030, 4016, 3899, 1247, 1855, 3919, 1244, 4140, 3930, 4117, 4189, 4001, 3643, 3619, 1240, 1575, 3871, 4135, 4408, 3604, 1274, 4149, 3741, 1938, 3709, 3622, 3834, 1983, 1743, 3979, 4461, 4113, 3817, 4232, 4214, 3902, 1219, 1486, 1512, 1516, 4047, 4082, 4094, 1574, 1248, 4078, 1872, 1426, 3785, 3687, 4318, 4243, 3866, 3704, 4172, 1513, 1856, 3324, 2769, 2678, 1561, 3296, 2348, 377, 1395, 1584, 4093, 4453, 3778, 4210, 4009, 4264, 4339, 1738, 4194, 4474, 4013, 4266, 1771, 1759, 1922, 3860, 3832, 1349, 1381, 3663, 1185, 3932, 3858, 1198, 4192, 3611, 1919, 3790, 3712, 4095, 1887, 4468, 1720, 4247, 934, 1790, 2002, 1657, 1336, 3894, 1554, 3576, 4314, 3695, 1850, 4362, 1815, 3656, 3638, 4454, 1443, 1661, 4504, 1359, 3650, 1305, 1648, 3831, 4435, 4451, 1993, 3849, 1718, 4068, 1835, 1425, 1428, 4380, 1658, 1452, 4024, 4072, 3791, 4491, 4323, 4014, 1304, 1511, 1981, 1676, 4422, 1283, 3861, 3644, 4259, 1749, 1418, 1176, 4216, 1924, 1421, 3992, 4423, 4253, 3748, 4119, 1297, 1299, 1954, 4450, 3990, 4029, 3711, 4344, 1578, 4384, 3892, 3674, 3595, 4405, 3973, 4109, 4159, 3744, 4387, 1960, 4275, 4492, 4365, 4118, 3655, 3886, 4278, 3751, 3678, 4353, 3175, 2970, 1228, 3534, 3437, 3406, 3641, 3639, 4470, 3941, 4348, 3587, 3818, 3814, 4350, 3821, 3929, 3824, 4465, 4494, 1398, 3828, 1284, 3773, 4209, 3014, 3884, 2783, 4038, 3545, 2916, 3112, 2834, 3880, 4399, 4048, 3752, 4207, 1235, 3943, 4355, 4087, 4100, 4163, 4486, 4178, 1643, 4152, 3634, 4445, 4303, 4284, 1213, 4382, 1399, 3714, 4487, 3615, 4018, 4420, 4121, 3724, 4282, 4310, 4488, 4127, 3670, 4052, 3627, 3777, 1371, 3685, 4482, 4258, 3812, 1581, 4124, 4508, 4500, 3652, 3972, 1309, 1528, 3717, 1217, 2740, 3125, 3546, 3842, 4404, 3690, 3962, 4329, 4410, 3703, 4136, 3589, 3907, 4153, 2707, 4180, 3923, 4444, 4361, 4394, 4302, 3758, 4510, 3742, 1710, 1335, 1666, 1407, 4187, 1193, 1188, 1196, 1724, 1717, 1200, 1997, 1701, 4280, 4006, 1984, 3833, 1593, 2561, 4442, 3900, 4298, 1761, 4388, 1597, 4317, 4249, 1341, 1203, 1680, 4441, 3628, 3725, 1945, 937, 4206, 1588, 1439, 3794, 4447, 3776, 3658, 4064, 4011, 4272, 4503, 1324, 1367, 3594, 3680, 1300, 1750, 3913, 3774, 1877, 1987, 3998, 1380, 4185, 3686, 3696, 4463, 3853, 4244, 4083, 1909, 3897, 1546, 3613, 3273, 3452, 3525, 1229, 3835, 1234, 3970, 3856, 3586, 1175, 4321, 2778, 4412, 4407, 1810, 3823, 1427, 1507, 1878, 4333, 3146, 4281, 4211, 1179, 3843, 1677, 1524, 1525, 1974, 1239, 4167, 3740, 1342, 1471, 4176, 1232, 1989, 4125, 1741, 1580, 4060, 1576, 1212, 3921, 1174, 4061, 4021, 3693, 3734, 1382, 3684, 3806, 3635, 3976, 3840, 4286, 3836, 4161, 3659, 3743, 3605, 4392, 4328, 1316, 4173, 4265, 1246, 1629, 1829, 3826, 1992, 1656, 1837, 1914, 1839, 3984, 3910, 4160, 4184, 4132, 4250, 1562, 1847, 1792, 3769, 1534, 4003, 1689, 1599, 1265, 1410, 1823, 1498, 1329, 386, 940, 1655, 3874, 4261, 3626, 3579, 1644, 3936, 4054, 4260, 4297, 4357, 4022, 4046, 1222, 1996, 3788, 3953, 4226, 4277, 4101, 3797, 4019, 3771, 3632, 1402, 4137, 3772, 3661, 4186, 4174, 1170, 3850, 1315, 1611, 1859, 4183, 1403, 4033, 3623, 3596, 1461, 1848, 1173, 1928, 1765, 3719, 3584, 3591, 4165, 4191, 3980, 4058, 4200, 4336, 3958, 1260, 4051, 3798, 4032, 1774, 4322, 3991, 4106, 3978, 3965, 4464, 3813, 4154, 1365, 3846, 4345, 4379, 3603, 1693, 3937, 3801, 1787, 1214, 4483, 4201, 1898, 1845, 1220, 4245, 1347, 3867, 1900, 3640, 3916, 4089, 3745, 3975, 4256, 4308, 4000, 3868, 4403, 3883, 4326, 4393, 3895, 4237, 3955, 4005, 4092, 4372, 4283, 3805, 4151, 3982, 3597, 3872, 3987, 4490, 3876, 3668, 4300, 1679, 1242, 3598, 3583, 3647, 4304, 3779, 1907, 4042, 3648, 3996, 3800, 4367, 4099, 1807, 3887, 4428, 1625, 1390, 1884, 1682, 4364, 4306, 3869, 1278, 942, 1505, 1542, 1709, 3600, 3914, 3948, 1865, 1716, 3961, 1506, 4221, 1376, 1683, 4276, 1721, 1632, 3607, 1455, 1913, 1497, 1317, 1951, 1778, 3768, 4225, 1668, 390, 4406, 4341, 1480, 392, 1714, 1422, 397, 3618, 4433, 1177, 3898, 398, 950, 400, 1965, 4471, 953, 401, 403, 955, 1482, 3602, 4434, 3721, 1592, 1825, 1698, 1519, 1352, 4409, 1977, 405, 1631, 1684, 409, 3889, 2117, 411, 413, 414, 415, 961, 1905, 416, 963, 3675, 1833, 3974, 965, 970, 1897, 1272, 971, 4133, 3830, 972, 424, 427, 1708, 4141, 3749, 1619, 975, 4044, 3590, 1944, 4126, 1923, 1706, 432, 976, 978, 4400, 433, 1606, 980, 2604, 2895, 436, 3448, 2188, 438, 1692, 982, 2243, 439, 442, 3862, 4055, 4358, 2276, 985, 1664, 443, 4309, 4045, 3854, 3682, 1972, 2688, 4366, 3425, 3231, 3649, 2759, 2724, 3755, 1216, 3599, 3775, 2754, 2606, 3471, 3234, 2565, 2803, 2562, 2684, 3411, 3405, 2690, 1255, 3694, 3985, 3535, 1306, 4084, 3282, 1353, 1780, 448, 2031, 990, 991, 1485, 992, 1728, 993, 4703, 994, 1696, 997, 998, 3116, 1869, 456, 1386, 4778, 2040, 1000, 4981, 3879, 2698, 460, 1001, 461, 1447, 1002, 1003, 463, 464, 4425, 465, 1004, 466, 469, 1231, 1468, 470, 2496, 1005, 476, 1273, 1744, 1488, 1659, 4598, 1604, 479, 1013, 1014, 481, 4313, 1499, 1016, 4073, 1777, 2172, 485, 4274, 4599, 4796, 487, 4710, 1775, 4205, 1526, 1697, 2000, 488, 1894, 1882, 1817, 1880, 490, 1018, 3917, 3616, 4311, 1800, 4473, 4085, 4401, 3783, 3765, 1538, 3870, 491, 3699, 3792, 4290, 2074, 492, 494, 2421, 4090, 2012, 1803, 3642, 1322, 1020, 502, 1333, 504, 4805, 1890, 507, 3608, 1732, 511, 2397, 1864, 1816, 1531, 513, 3636, 514, 1699, 1027, 4987, 1366, 4664, 4950, 4027, 3478, 2959, 1293, 3197, 1595, 3413, 518, 4698, 1032, 1033, 1675, 1797, 4578, 4854, 2315, 521, 1999, 3759, 1215, 4122, 1730, 3966, 1035, 4973, 528, 3911, 3289, 1821, 530, 2426, 532, 1874, 533, 1038, 538, 539, 3767, 1929, 3683, 2100, 4853, 1338, 2021, 1616, 1901, 1796, 1517, 4331, 4332, 4466, 2719, 4123, 4233, 1824, 3129, 3087, 3505, 4158, 4254, 3720, 4351, 4414, 3453, 3254, 1649, 544, 4240, 3580, 1046, 548, 549, 2598, 552, 553, 3819, 1259, 4062, 554, 2841, 3515, 1433, 1323, 4301, 555, 558, 3738, 1567, 559, 1054, 1270, 3857, 2175, 1257, 1238, 3662, 561, 1058, 563, 4872, 2097, 1061, 4203, 1899, 3981, 1062, 1303, 4116, 570, 1946, 3620, 1514, 4002, 3679, 3964, 3707, 3666, 1348, 4273, 1804, 4497, 571, 1401, 4438, 1895, 1264, 1912, 573, 3939, 4170, 4056, 1271, 1521, 1221, 1451, 3934, 4080, 4352, 1700, 3739, 1419, 1811, 3950, 1690, 3716, 1569, 1437, 1674, 4337, 1285, 4268, 1183, 3881, 3938, 4198, 4416, 4229, 4292, 4193, 4325, 4375, 4432, 4215, 3944, 1409, 4195, 4223, 1540, 4455, 4512, 1723, 3750, 4070, 4104, 4235, 1863, 4346, 1881, 1071, 1814, 4443, 3761, 1346, 1587, 3592, 576, 4469, 1275, 1950, 4129, 1296, 1551, 3733, 4111, 3993, 4108, 3949, 1287, 1713, 1556, 1736, 1995, 1705, 1861, 4120, 4417, 1292, 1785, 1236, 4293, 1827, 3839, 3983, 4395, 1957, 3928, 4295, 3715, 3855, 3609, 4076, 4175, 3927, 1757, 4299, 1773, 1364, 1462, 4007, 3729, 3665, 3722, 1691, 1712, 1624, 1751, 1394, 3924, 3952, 581, 2003, 1559, 1250, 1555, 4114, 1818, 1076, 4448, 1078, 1786, 3354, 1832, 3432, 2891, 4583, 1903, 1080, 4459, 589, 1563, 1084, 591, 592, 594, 595, 1858, 596, 598, 1453, 2559, 2590, 2738, 4478, 4069, 1809, 1327, 4150, 3260, 3481, 3672, 3625, 4439, 3852, 3997, 3847, 4050, 3377, 4424, 4143, 4493, 3492, 1412, 601, 4017, 4477, 1794, 602, 4263, 3072, 2009, 1754, 1088, 3888, 603, 604, 1368, 3827, 4103, 1571, 1438, 2069, 611, 1093, 612, 1094, 3701, 4389, 614, 5000, 615, 1332, 1715, 4369, 1652, 1541, 1921, 1733, 1373, 1870, 619, 4218, 1798, 1702, 1384, 3700, 1788, 1600, 624, 2473, 1423, 2771, 1339, 3875, 3070, 2015, 2557, 2795, 2906, 2103, 3299, 3118, 4838, 4965, 4605, 4576, 1103, 1614, 1947, 631, 4479, 3766, 1107, 635, 1813, 636, 2431, 637, 1109, 4577, 639, 3706, 1110, 640, 1111, 4581, 4644, 4734, 642, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 1269, 3095, 1286, 3624, 4481, 3511, 2735, 4381, 3295, 1585, 1568, 1766, 3221, 2643, 2739, 4629, 3920, 4603, 2307, 647, 1801, 4279, 3762, 1637, 1509, 4590, 648, 1117, 4800, 4724, 4674, 1119, 2965, 2695, 3293, 4722, 1121, 4784, 653, 1122, 3264, 655, 656, 657, 4035, 2910, 3391, 2808, 3099, 2913, 3676, 2852, 3284, 2731, 3537, 3994, 1319, 4467, 1623, 660, 4714, 4812, 1123, 664, 2138, 1126, 1128, 1129, 1130, 670, 1527, 671, 1772, 1133, 2196, 1136, 1137, 5005, 1933, 1731, 1793, 3342, 1140, 1141, 4525, 687, 3822, 1601, 1148, 693, 3905, 694, 1414, 1149, 4756, 4859, 2258, 697, 1795, 1372, 1695, 1866, 1151, 4115, 1719, 2194, 1920, 4999, 1544, 703, 4031, 4305, 1636, 1393, 3810, 4452, 3677, 4227, 4402, 4157, 4197, 1337, 1253, 1532, 4028, 3989, 4715, 704, 1493, 1902, 1152, 4824, 4182, 707, 708, 709, 4732, 4476, 1375, 713, 714, 715, 1841, 4234, 1154, 1314, 4096, 4895, 718, 719, 720, 2063, 1311, 721, 4903, 4885, 4217, 1627, 2116, 1158, 724, 5013, 4894, 1953, 1201, 3787, 729, 2485, 3747, 730, 1846, 3815, 1964, 1822, 1262, 4480, 1537, 1291, 1318, 4976, 4294, 1582, 4587, 3439, 2833, 4287, 4509, 4505, 4267, 3697, 3272, 4112, 2967, 3651, 1805, 3705, 1195, 3859, 1164, 1165, 2351, 4835, 4571, 2423, 1745, 1973, 1168, 3807, 4307, 1169, 1454, or 1781. In some aspects, the 581 to 589 region of the VP capsid polypeptide has a sequence of any one of SEQ ID NOs: 4177, 1289, 740, 3477, 1548, 3051, 2982, 2877, 3036, 3426, 3073, 3169, 747, 3362, 2784, 21, 3192, 1638, 748, 1703, 1836, 3660, 29, 750, 1640, 33, 35, 36, 1205, 3795, 753, 4288, 4219, 2770, 2962, 4241, 4107, 2718, 3317, 4391, 3263, 2553, 3947, 2710, 2608, 3467, 2796, 2961, 2529, 3320, 2674, 2635, 3446, 3053, 3250, 3688, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 3327, 3178, 3006, 3252, 2991, 3470, 2888, 2572, 3418, 2642, 2519, 3363, 40, 2633, 45, 756, 48, 1952, 3122, 2551, 3718, 3484, 3577, 759, 3960, 1378, 2994, 3080, 2589, 3681, 760, 762, 3925, 3654, 60, 4086, 1740, 1660, 62, 1966, 63, 768, 66, 67, 68, 3816, 2599, 2706, 74, 3865, 1549, 1361, 2453, 3882, 3713, 1942, 1312, 4501, 76, 1560, 4289, 3757, 3593, 3908, 4397, 4415, 3723, 3754, 4360, 3945, 2854, 3523, 3669, 4065, 1294, 1484, 3891, 4456, 4040, 4496, 1982, 2001, 1478, 1189, 1490, 1885, 1926, 89, 1180, 92, 786, 94, 95, 1831, 1867, 4458, 97, 1970, 1936, 1590, 99, 789, 790, 1298, 101, 1688, 791, 792, 2007, 103, 104, 105, 108, 1579, 110, 1906, 1444, 1344, 116, 802, 117, 118, 121, 803, 1463, 124, 807, 133, 1978, 812, 135, 136, 137, 138, 140, 1326, 141, 142, 143, 1634, 1302, 817, 818, 147, 148, 149, 151, 154, 155, 819, 820, 157, 165, 822, 168, 169, 170, 823, 1651, 173, 1483, 174, 825, 176, 180, 827, 181, 828, 184, 185, 186, 188, 189, 830, 192, 193, 194, 196, 1391, 197, 200, 201, 202, 203, 205, 832, 206, 834, 835, 836, 1334, 3901, 3312, 2649, 1450, 4386, 3028, 1446, 215, 1522, 3490, 217, 219, 1612, 220, 221, 222, 839, 223, 840, 224, 230, 231, 842, 232, 233, 235, 851, 853, 1536, 240, 242, 243, 244, 245, 246, 247, 250, 251, 254, 255, 857, 256, 258, 259, 859, 260, 861, 862, 2326, 264, 1241, 266, 1477, 867, 869, 870, 271, 871, 872, 272, 1252, 1591, 275, 1980, 276, 876, 877, 277, 278, 1892, 1539, 3601, 3877, 1930, 1460, 282, 1799, 1626, 1374, 1620, 1290, 1354, 1956, 889, 292, 4324, 4138, 891, 3903, 297, 3829, 3809, 4271, 3844, 1436, 1617, 1961, 304, 1589, 4370, 3646, 4059, 1431, 1331, 312, 1779, 313, 896, 1558, 1552, 322, 1958, 324, 901, 1491, 329, 330, 4484, 1756, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 4248, 1557, 1602, 4188, 1330, 4349, 3337, 3533, 2629, 3038, 2539, 3567, 3291, 3348, 3115, 3359, 2993, 3079, 3506, 2527, 2963, 2938, 2516, 3113, 2639, 3143, 3044, 2747, 3154, 3280, 3039, 2743, 2902, 3056, 2554, 2522, 3103, 3210, 3434, 2656, 4251, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 2869, 2610, 3298, 2775, 3108, 2603, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3629, 3578, 3885, 2817, 3248, 2954, 3334, 3571, 3345, 3551, 3384, 3422, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3120, 3286, 2843, 3465, 3946, 4222, 2682, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 4269, 3487, 3489, 3019, 1266, 3435, 3482, 3020, 2748, 2646, 2787, 2760, 2563, 3085, 3133, 3158, 3170, 2703, 3494, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 3689, 2968, 1457, 3450, 2577, 2567, 2532, 3415, 2934, 2984, 2568, 4147, 3002, 2986, 2979, 2542, 1762, 4472, 1529, 4057, 1976, 4507, 1583, 1711, 2013, 1594, 4440, 344, 921, 4498, 3728, 3637, 3570, 3429, 4091, 2870, 2995, 4338, 3205, 2880, 3261, 3968, 4291, 4204, 4071, 4034, 1742, 2820, 3912, 3287, 4145, 354, 1782, 927, 3610, 4224, 1918, 1959, 3736, 1770, 931, 4155, 364, 1279, 365, 1543, 367, 4049, 1633, 1722, 369, 1495, 1566, 1211, 1748, 4142, 1434, 1358, 1191, 3732, 4110, 4039, 4411, 4506, 4156, 4036, 4030, 4016, 3899, 1247, 1855, 3919, 1244, 4140, 3930, 4117, 4189, 4001, 3643, 3619, 1240, 1575, 3871, 4135, 4408, 3604, 1274, 3741, 1938, 3709, 3622, 3834, 1983, 1743, 3979, 4461, 4113, 3817, 4232, 4214, 1219, 1486, 1512, 1516, 4047, 4082, 4094, 1574, 1248, 4078, 1872, 1426, 3785, 3687, 4318, 4243, 3866, 3704, 4172, 1513, 1856, 3324, 2769, 2678, 1561, 3296, 377, 1395, 4093, 4453, 3778, 4210, 4009, 4264, 4339, 1738, 4194, 4474, 4013, 4266, 1771, 3860, 3832, 1349, 1381, 3663, 1185, 3932, 3858, 1198, 4192, 3611, 1919, 3790, 3712, 4095, 1887, 4468, 1720, 4247, 2002, 1657, 1336, 3894, 1554, 3576, 4314, 3695, 4362, 1815, 3656, 3638, 4454, 1443, 1661, 1359, 3650, 1648, 3831, 4435, 4451, 1993, 3849, 1718, 4068, 1835, 1425, 1428, 4380, 1658, 1452, 4024, 4072, 3791, 4491, 4323, 4014, 1304, 1511, 1981, 1676, 4422, 3748, 4119, 1297, 1299, 4450, 3990, 4029, 3711, 4344, 1578, 4384, 3892, 3674, 3595, 4405, 3973, 4109, 4159, 3744, 4387, 1960, 4275, 4492, 4365, 4118, 3655, 3886, 4278, 3751, 3678, 4353, 3175, 2970, 1228, 3534, 3437, 3406, 3641, 3639, 4470, 3941, 4348, 3587, 3818, 3814, 4350, 3821, 3929, 3824, 4465, 4494, 1398, 3828, 1284, 3773, 4209, 3014, 3884, 2783, 4038, 3545, 2916, 3112, 2834, 3880, 4399, 4048, 3752, 4207, 1235, 3943, 4355, 4087, 4100, 4163, 4486, 4178, 1643, 4152, 3634, 4445, 4303, 4284, 1213, 4382, 1399, 3714, 4487, 3615, 4018, 4420, 4121, 3724, 4282, 4310, 4488, 4127, 3670, 4052, 4124, 4508, 4500, 3652, 3972, 1309, 1528, 3717, 1217, 2740, 3125, 3546, 3842, 4404, 3690, 3962, 4329, 4410, 3703, 4136, 3589, 3907, 4153, 2707, 4180, 3923, 4444, 4361, 4394, 4302, 3758, 4510, 3742, 1710, 1335, 1997, 1701, 4280, 4006, 1984, 3833, 1593, 2561, 4442, 3900, 4298, 1761, 4388, 1597, 4317, 4249, 1341, 1680, 4441, 3628, 3725, 1945, 937, 4206, 1588, 1439, 3794, 4447, 3776, 3658, 4064, 4011, 4272, 4503, 1367, 3594, 3680, 1300, 1750, 3913, 3774, 1877, 1987, 3998, 1380, 4185, 3686, 3696, 4463, 3853, 4244, 4083, 1909, 3897, 1546, 3613, 3273, 3452, 3525, 1229, 3835, 1234, 3970, 3856, 3586, 1175, 4321, 2778, 4412, 4407, 1810, 3823, 1427, 1507, 1878, 4333, 3146, 4281, 4211, 1179, 3843, 1677, 1524, 1525, 1974, 1239, 4167, 3740, 1342, 4125, 1580, 4060, 1576, 1212, 3921, 1174, 4061, 4021, 3693, 3734, 1382, 3684, 3806, 3635, 3976, 3840, 4286, 3836, 4161, 3659, 3743, 3605, 4392, 4328, 1316, 4173, 4265, 1246, 1629, 1829, 1837, 1914, 1839, 3984, 3910, 4160, 4184, 4132, 4250, 1562, 1847, 1792, 3769, 1534, 4003, 1689, 1599, 1265, 1655, 3874, 4261, 3626, 3579, 1644, 3936, 4054, 4260, 4297, 4357, 4022, 4046, 1222, 1996, 3788, 3953, 4226, 4277, 4101, 3797, 4019, 3771, 3632, 1402, 4137, 3772, 3661, 4186, 4174, 1170, 3850, 1315, 1611, 4033, 3623, 3596, 1461, 1848, 1173, 1928, 1765, 3719, 3584, 3591, 4165, 4191, 3980, 4058, 4200, 4336, 3958, 1260, 4051, 3798, 4032, 1774, 4322, 3991, 4106, 3978, 3965, 4464, 3813, 4154, 1365, 3846, 4345, 4379, 3603, 1693, 3937, 3801, 1787, 1214, 4483, 4201, 1898, 1220, 4245, 1347, 3867, 1900, 3640, 3916, 4089, 3745, 3975, 4256, 4308, 4000, 3868, 4403, 3883, 4326, 4393, 3895, 4237, 3955, 4005, 4092, 4372, 4283, 3805, 4151, 3982, 3597, 3872, 3987, 4490, 3876, 3668, 4300, 1679, 1242, 3598, 3583, 3647, 4304, 3779, 1907, 4042, 3648, 3996, 3800, 4367, 4099, 1807, 3887, 4428, 1625, 1390, 1884, 1682, 4364, 4306, 3869, 1278, 942, 1505, 1542, 1709, 3600, 3914, 3948, 1865, 1716, 3961, 1506, 4221, 1376, 1683, 4276, 1721, 1632, 3607, 1455, 1913, 1497, 1317, 1951, 1778, 3768, 4225, 1668, 390, 4406, 4341, 1480, 1714, 1422, 397, 3618, 4433, 1177, 3898, 398, 950, 400, 1965, 4471, 403, 1482, 3602, 4434, 3721, 1592, 1519, 1352, 4409, 1977, 1631, 1684, 409, 3889, 414, 415, 961, 1905, 416, 963, 3675, 1833, 3974, 965, 970, 1897, 4133, 3830, 972, 424, 427, 1708, 4141, 3749, 1619, 975, 4044, 3590, 1944, 4126, 1923, 1706, 4400, 1606, 2604, 2895, 436, 3448, 1692, 3862, 4055, 4358, 985, 1664, 4045, 3854, 3682, 1972, 2688, 4366, 3425, 3231, 3649, 2759, 2724, 3755, 1216, 3599, 3775, 2754, 2606, 3471, 3234, 2565, 2803, 2562, 2684, 3411, 3405, 2690, 1255, 3694, 3535, 1306, 4084, 3282, 1780, 990, 991, 992, 1728, 994, 1696, 998, 3116, 1869, 456, 1386, 3879, 2698, 1001, 461, 1447, 464, 4425, 1231, 1468, 476, 1273, 1744, 1488, 1659, 1604, 481, 4313, 1499, 1016, 4073, 1777, 4274, 487, 1775, 4205, 1526, 1697, 2000, 1882, 1817, 1880, 490, 1018, 3917, 3616, 4311, 1800, 4473, 4085, 4401, 3783, 3765, 491, 3699, 3792, 4290, 4090, 2012, 1803, 3642, 1322, 502, 1333, 1890, 3608, 1732, 511, 2397, 1864, 1816, 1531, 3636, 1699, 1027, 1366, 4027, 3478, 2959, 3197, 1595, 3413, 518, 1675, 1797, 2315, 1999, 3759, 1215, 4122, 1730, 3966, 3911, 3289, 1821, 530, 1874, 1038, 3767, 1929, 3683, 1338, 1517, 4331, 4332, 4466, 2719, 4123, 4233, 1824, 3129, 3087, 3505, 4158, 4254, 3720, 4351, 4414, 3453, 3254, 1649, 544, 4240, 3580, 1046, 2598, 552, 3819, 1259, 4062, 2841, 3515, 4301, 3738, 1567, 1270, 3857, 1257, 1238, 3662, 561, 1058, 1061, 4203, 1899, 3981, 1062, 1303, 4116, 1946, 3620, 1514, 4002, 3679, 3964, 3707, 3666, 1348, 4273, 1804, 4497, 1401, 4438, 1895, 1264, 1912, 3939, 4170, 4056, 1271, 1521, 1221, 1451, 3934, 4080, 4352, 1700, 3739, 1419, 1811, 3950, 1690, 3716, 1437, 1674, 4337, 1285, 4268, 1183, 3881, 3938, 4198, 4416, 4229, 4292, 4193, 4325, 4375, 4432, 4215, 3944, 1409, 4195, 4223, 1540, 1723, 3750, 4070, 4104, 4235, 1863, 4346, 1881, 1814, 4443, 3761, 1346, 1587, 3592, 576, 4469, 1275, 1950, 4129, 1296, 1551, 3733, 4111, 3993, 4108, 3949, 1287, 1713, 1736, 1995, 1705, 1861, 4120, 4417, 1785, 1236, 4293, 3839, 3983, 4395, 1957, 3928, 4295, 3715, 3855, 3609, 4076, 4175, 3927, 1757, 4299, 1773, 1364, 1462, 4007, 3729, 3665, 3722, 1691, 1624, 1751, 1394, 3924, 3952, 581, 2003, 1559, 1250, 1555, 4114, 1818, 1076, 4448, 1078, 1786, 3354, 1832, 3432, 2891, 1903, 4459, 1563, 591, 594, 598, 1453, 2559, 2590, 2738, 4478, 4069, 1809, 1327, 4150, 3260, 3481, 3672, 3625, 4439, 3852, 3997, 3847, 4050, 3377, 4424, 4143, 4493, 3492, 601, 4017, 4477, 1794, 4263, 3072, 2009, 1754, 1088, 3888, 1368, 3827, 4103, 1571, 1438, 1093, 612, 1094, 3701, 4389, 1332, 1715, 4369, 1652, 1541, 1921, 1733, 1373, 1870, 619, 4218, 1798, 1702, 1384, 3700, 1788, 1600, 624, 2771, 1339, 3875, 3070, 2557, 2795, 2906, 3118, 1614, 1947, 4479, 3766, 635, 1813, 636, 3706, 1110, 640, 1111, 642, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 3095, 1286, 3624, 4481, 3511, 2735, 4381, 3295, 1585, 1766, 3221, 2643, 2739, 3920, 2307, 647, 1801, 4279, 3762, 1637, 1509, 1119, 2965, 2695, 3293, 1121, 1122, 3264, 655, 4035, 2910, 3391, 2808, 3099, 2913, 3676, 2852, 3284, 2731, 3994, 1319, 4467, 1623, 664, 1128, 1129, 1130, 670, 1527, 671, 1772, 5005, 1933, 1731, 1793, 3342, 3822, 1601, 3905, 694, 1414, 1795, 1372, 1695, 1866, 4115, 1719, 1920, 1544, 703, 4031, 4305, 1636, 1393, 3810, 4452, 3677, 4227, 4402, 4157, 4197, 1337, 1253, 1532, 4028, 3989, 1493, 1902, 4182, 707, 708, 4476, 714, 1841, 4234, 1314, 4096, 4217, 1627, 1953, 1201, 3787, 3747, 730, 1846, 3815, 1964, 1822, 1262, 4480, 1537, 1291, 1318, 4294, 1582, 3439, 2833, 4287, 4509, 4505, 4267, 3697, 3272, 4112, 2967, 3651, 3705, 1195, 3859, 1164, 1165, 1745, 1973, 1168, 3807, 4307, 1454, or 1781.

In some aspects, the basic residue is selected from the group consisting of K, R, and H.

In some aspects, X³ of SEQ ID NO: 5014 is K or R. In some aspects, the 581 to 589 region of the VP capsid polypeptide has a sequence of any one of SEQ ID NOs: 4177, 1289, 740, 741, 18, 745, 3477, 1548, 3051, 2982, 2877, 3036, 3426, 3073, 3169, 747, 3362, 2784, 21, 3192, 23, 1638, 748, 1703, 1836, 3660, 29, 750, 1640, 33, 34, 35, 36, 37, 38, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 3327, 3178, 3006, 3252, 2991, 3470, 2888, 2572, 3418, 2642, 2519, 3363, 40, 2633, 41, 42, 43, 755, 45, 756, 46, 48, 1952, 758, 49, 3122, 2551, 3718, 3484, 3577, 759, 1230, 3960, 1378, 2994, 3080, 2589, 3681, 760, 53, 3925, 55, 3654, 56, 61, 4086, 1740, 1660, 765, 766, 62, 1966, 63, 64, 768, 771, 65, 66, 67, 68, 3816, 2599, 2706, 74, 3865, 1549, 1361, 3882, 3713, 1942, 1312, 4501, 76, 77, 1560, 4289, 3757, 79, 3593, 4043, 1806, 3908, 4397, 1615, 4415, 3723, 2006, 3754, 1470, 4360, 3945, 2854, 3523, 3669, 82, 4065, 83, 1294, 1484, 3891, 4456, 4040, 4496, 1982, 2001, 1478, 1189, 1490, 1885, 1926, 89, 90, 91, 779, 1180, 92, 785, 786, 94, 95, 1831, 1867, 4458, 97, 1970, 1936, 1590, 100, 789, 790, 1298, 101, 1688, 791, 792, 2007, 103, 794, 795, 104, 105, 106, 1849, 108, 1579, 110, 1906, 113, 1444, 1344, 116, 802, 117, 118, 121, 803, 804, 1463, 805, 122, 124, 125, 127, 807, 132, 809, 810, 133, 1978, 812, 135, 136, 137, 138, 813, 140, 815, 1326, 141, 142, 143, 144, 145, 1634, 1302, 817, 146, 818, 147, 148, 149, 151, 152, 153, 154, 155, 819, 820, 157, 159, 160, 163, 164, 165, 166, 822, 167, 168, 169, 170, 171, 823, 824, 1651, 173, 1483, 174, 175, 825, 176, 177, 826, 827, 181, 828, 182, 829, 184, 185, 186, 187, 188, 189, 830, 191, 192, 193, 194, 195, 196, 1391, 197, 200, 201, 202, 203, 204, 205, 832, 833, 206, 834, 836, 1334, 837, 3901, 3312, 2649, 1301, 211, 212, 1450, 4386, 3028, 213, 1446, 214, 215, 1896, 1522, 3490, 838, 217, 218, 219, 1612, 220, 221, 222, 839, 223, 840, 224, 226, 227, 230, 231, 842, 232, 233, 844, 236, 237, 851, 852, 853, 1536, 240, 854, 242, 243, 244, 245, 246, 247, 249, 250, 251, 252, 253, 254, 255, 857, 256, 257, 258, 259, 858, 859, 260, 261, 860, 861, 862, 262, 264, 1241, 265, 266, 866, 1477, 267, 867, 869, 870, 271, 871, 872, 272, 1252, 273, 1591, 275, 1980, 276, 876, 877, 277, 278, 279, 1892, 1539, 880, 1295, 3601, 3877, 1930, 1420, 1678, 1460, 282, 1799, 283, 1626, 1374, 287, 1620, 1290, 1354, 888, 291, 1956, 889, 292, 4324, 293, 4138, 891, 3903, 297, 3829, 3809, 4271, 3844, 1436, 1515, 1617, 1961, 304, 306, 1589, 4370, 308, 309, 894, 3646, 4059, 1431, 1331, 312, 895, 1779, 313, 314, 315, 316, 896, 1558, 1552, 898, 321, 322, 899, 1958, 901, 325, 902, 326, 1491, 329, 330, 905, 4484, 1756, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 1888, 4248, 1557, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 3435, 3482, 3020, 2748, 2646, 2787, 2760, 2563, 3085, 3133, 3158, 3170, 2703, 3494, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 2979, 2542, 1762, 4472, 1472, 1529, 4057, 1976, 4507, 1583, 338, 911, 1711, 340, 912, 2013, 341, 4440, 917, 344, 918, 919, 921, 922, 347, 351, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 4145, 354, 925, 1782, 926, 927, 3610, 357, 359, 4224, 1918, 1959, 3736, 1770, 931, 4155, 364, 1279, 365, 1543, 367, 4049, 1633, 1722, 368, 369, 1495, 1566, 370, 1211, 1873, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 3741, 1938, 3709, 1727, 3893, 1280, 3622, 1784, 3834, 1752, 1983, 1743, 3979, 4461, 4113, 3817, 4232, 1340, 1489, 4214, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 377, 1395, 4453, 3940, 3778, 4374, 4210, 4009, 4264, 4339, 4368, 1738, 378, 1610, 4194, 4474, 4013, 4266, 1771, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 2002, 1657, 1663, 1336, 3894, 1554, 1494, 1405, 3576, 4314, 3695, 1851, 4362, 1815, 3845, 4511, 3656, 3638, 1406, 1596, 4454, 1443, 1661, 1359, 1734, 3650, 1910, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1680, 4441, 3628, 3725, 1945, 4206, 1588, 1439, 3794, 4447, 1838, 3776, 3658, 4064, 1377, 4011, 4272, 4503, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 3613, 3273, 3452, 3525, 1229, 4105, 3835, 383, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 1520, 1767, 1466, 1194, 4137, 1908, 1927, 1758, 3772, 3661, 4186, 4174, 1170, 3850, 1315, 1611, 1530, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 1716, 3961, 1506, 4221, 1376, 1683, 4276, 1445, 1721, 1632, 3607, 1328, 1455, 1913, 1497, 1317, 1951, 1778, 1379, 3768, 4225, 1668, 390, 391, 4406, 4341, 1480, 395, 1714, 1422, 396, 397, 3618, 4433, 1177, 3898, 398, 949, 950, 399, 951, 400, 1965, 4471, 952, 402, 2475, 403, 404, 4139, 1482, 3602, 4434, 3721, 1592, 1519, 1352, 4202, 4409, 1977, 1917, 407, 408, 1631, 1684, 409, 3889, 958, 959, 412, 960, 414, 415, 961, 1905, 416, 963, 417, 3675, 1833, 3974, 420, 421, 965, 966, 969, 970, 422, 1897, 423, 4133, 3830, 972, 424, 973, 427, 1708, 4141, 3749, 431, 1619, 975, 4044, 3590, 1944, 4126, 1923, 1706, 4400, 434, 1606, 979, 2604, 2895, 436, 3448, 1692, 983, 3862, 4055, 4358, 984, 985, 1664, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 3535, 1306, 4084, 3282, 1780, 987, 988, 990, 451, 452, 991, 992, 1728, 994, 995, 454, 1696, 3116, 1869, 456, 1386, 999, 3879, 2698, 1001, 461, 1447, 462, 464, 4425, 1645, 1231, 1468, 472, 473, 476, 1273, 1007, 1744, 1488, 1659, 477, 1604, 481, 4313, 482, 483, 1499, 484, 1016, 4073, 1777, 1458, 4274, 487, 1775, 4205, 1526, 1697, 1017, 2000, 489, 3917, 3616, 4311, 1800, 4473, 4085, 4401, 1310, 3783, 3765, 491, 1948, 3699, 1735, 3792, 4290, 495, 4090, 496, 2012, 1803, 3642, 1322, 498, 1021, 1022, 502, 1333, 503, 1023, 1890, 510, 1026, 3608, 1732, 511, 2397, 1864, 1816, 1531, 3636, 1699, 1027, 1029, 1366, 2112, 1261, 4027, 3478, 3763, 2959, 517, 3197, 1595, 3413, 1448, 518, 1675, 1797, 520, 1999, 3759, 1215, 4122, 1730, 524, 526, 3966, 3911, 529, 3289, 1821, 530, 531, 1874, 1038, 536, 537, 3767, 1929, 1041, 1042, 3683, 542, 1338, 4331, 4332, 4466, 2719, 3803, 4123, 4233, 1824, 3129, 3087, 3505, 4158, 4254, 1449, 3720, 4351, 4414, 1673, 3453, 3254, 1649, 546, 4240, 3580, 1046, 1047, 550, 2598, 551, 552, 3819, 1259, 4062, 2841, 1050, 3515, 4301, 1052, 1053, 3738, 1567, 1270, 3857, 1055, 1257, 1238, 3662, 561, 562, 1058, 4203, 1899, 3981, 1062, 1303, 4116, 567, 1946, 3620, 1514, 4002, 3679, 3964, 3707, 3666, 1348, 4273, 1804, 4497, 1401, 4438, 1895, 1264, 572, 1912, 3939, 4170, 4056, 1271, 1065, 1521, 1221, 1451, 1654, 3934, 4080, 4352, 1700, 3739, 1565, 1419, 1811, 3950, 1690, 1397, 3716, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 1723, 1070, 3750, 3633, 4070, 4104, 4162, 4235, 1863, 4346, 1881, 1814, 1572, 4443, 3761, 1346, 1587, 1072, 3592, 576, 1891, 4469, 1275, 1950, 1963, 4129, 1296, 1551, 3733, 4111, 3993, 4108, 3949, 1287, 1713, 1736, 1995, 1435, 1424, 1830, 1705, 1861, 1197, 1254, 4120, 1545, 4417, 1469, 1785, 1236, 4293, 3839, 3983, 4395, 1957, 3928, 4295, 3715, 3855, 3609, 1990, 4076, 4175, 3927, 1757, 1650, 4299, 1773, 2004, 1267, 1364, 1462, 1879, 4007, 3729, 3665, 3722, 1691, 1624, 1751, 1394, 3924, 3952, 580, 581, 2003, 1343, 1559, 1250, 1555, 4114, 1818, 1075, 1076, 1077, 1704, 4448, 1078, 1079, 1786, 585, 3354, 1832, 3432, 2891, 2505, 1903, 1081, 1082, 4459, 1083, 1563, 590, 591, 1085, 594, 1453, 2559, 2590, 2738, 4478, 4069, 1809, 1327, 4150, 3260, 3481, 3672, 3625, 2008, 4439, 3852, 3997, 1411, 599, 3847, 1268, 4050, 3377, 4424, 1496, 4143, 4493, 3492, 1962, 600, 601, 4017, 4477, 1794, 4263, 3072, 2009, 1754, 1087, 1088, 3888, 1368, 3827, 4103, 606, 1090, 1571, 1091, 1438, 1092, 1093, 612, 1094, 3701, 4389, 613, 1603, 1332, 1715, 4369, 1652, 1096, 617, 1541, 618, 1921, 1733, 1373, 1870, 619, 4218, 1798, 1100, 1702, 1101, 1667, 1384, 623, 3700, 1788, 1600, 624, 626, 2771, 627, 1339, 3875, 628, 629, 3070, 4860, 2557, 2795, 2906, 3118, 1614, 1947, 4479, 3766, 632, 1104, 1105, 633, 1813, 636, 3706, 1110, 640, 1111, 641, 642, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 3095, 1286, 3624, 4481, 1432, 3511, 2735, 4381, 3295, 1585, 1114, 1766, 3221, 2643, 2739, 3920, 644, 1116, 645, 646, 647, 1801, 4279, 3762, 1637, 1509, 1119, 2965, 2369, 2695, 3293, 650, 651, 1120, 652, 1121, 3264, 655, 658, 659, 4035, 2910, 3391, 2808, 3099, 2913, 3676, 2852, 3284, 2731, 1609, 3994, 1319, 4467, 1802, 1227, 1623, 662, 663, 664, 1124, 1125, 1128, 1129, 668, 669, 1130, 670, 1131, 1527, 1132, 671, 1772, 1135, 672, 1933, 1731, 1793, 675, 676, 3342, 1138, 1139, 677, 679, 1142, 1143, 681, 1145, 682, 685, 3822, 689, 690, 691, 1147, 1601, 3905, 694, 695, 1414, 1150, 1795, 1372, 1695, 1866, 4115, 1719, 698, 1920, 699, 700, 1544, 4305, 1636, 1393, 2010, 3810, 4452, 3677, 4227, 1277, 1400, 4402, 4157, 4197, 1337, 1253, 1532, 4028, 3989, 1493, 1854, 1902, 1510, 4182, 706, 707, 708, 4476, 1245, 714, 1841, 4234, 1314, 4096, 717, 1155, 4217, 1156, 723, 1157, 1627, 725, 1159, 1953, 1160, 1201, 727, 3787, 3747, 730, 1846, 3815, 2005, 1964, 1822, 1262, 4480, 1537, 1291, 1318, 4294, 1582, 734, 3439, 2833, 4287, 4509, 4505, 4267, 3697, 3272, 4112, 2967, 3651, 1628, 3705, 1195, 3859, 1164, 1165, 1166, 1745, 1973, 1168, 3807, 4307, 1454, or 1781.

In some aspects, X⁶ of SEQ ID NO: 5014 is K or R. In some aspects, the 581 to 589 region of the VP capsid polypeptide has a sequence of any one of SEQ ID NOs: 738, 4177, 1289, 740, 14, 743, 744, 17, 3477, 1548, 3051, 2982, 2877, 746, 19, 3036, 3426, 3073, 3169, 747, 3362, 2784, 21, 3192, 22, 1638, 748, 24, 749, 27, 1703, 1836, 28, 3660, 29, 750, 1640, 33, 35, 36, 1653, 1205, 3795, 753, 4288, 4219, 2770, 2962, 4241, 4107, 2718, 3317, 4391, 3263, 2553, 3947, 2710, 2608, 3467, 2796, 2961, 2529, 3320, 2674, 2635, 3446, 3053, 3250, 3688, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 1467, 3327, 3178, 3006, 3252, 2991, 3470, 2888, 2572, 3418, 2642, 2519, 3363, 40, 2633, 44, 45, 756, 47, 48, 1952, 3122, 2551, 3718, 3484, 3577, 1308, 3960, 1378, 2994, 3080, 2589, 3681, 51, 760, 762, 3925, 3654, 763, 59, 60, 4086, 1740, 1660, 62, 1966, 63, 2438, 768, 66, 67, 68, 3816, 2599, 2706, 72, 73, 74, 3865, 1549, 1361, 3882, 773, 3713, 1942, 1312, 4501, 76, 1413, 775, 1560, 4289, 3757, 3593, 3908, 4397, 4415, 3723, 3754, 4360, 3945, 2854, 3523, 3669, 1356, 4065, 1294, 1484, 85, 86, 3891, 4456, 4040, 4496, 1982, 2001, 1478, 1189, 1490, 1885, 1926, 89, 780, 1180, 92, 781, 782, 783, 786, 94, 95, 788, 1831, 1867, 96, 4458, 97, 1970, 1936, 1590, 98, 99, 789, 790, 1298, 1688, 791, 792, 2007, 102, 103, 104, 105, 107, 108, 1579, 1906, 111, 798, 112, 799, 800, 1444, 801, 114, 115, 1344, 116, 802, 117, 118, 119, 121, 803, 1463, 1760, 806, 126, 128, 807, 129, 131, 808, 133, 1978, 811, 812, 135, 136, 137, 138, 814, 140, 1326, 141, 142, 143, 2344, 816, 1634, 1302, 817, 818, 147, 148, 149, 150, 151, 154, 155, 819, 820, 156, 157, 158, 161, 162, 165, 822, 168, 169, 170, 823, 172, 1651, 173, 1483, 174, 825, 176, 178, 180, 827, 181, 828, 4911, 184, 186, 188, 189, 830, 192, 193, 194, 196, 1391, 198, 199, 200, 201, 202, 203, 205, 832, 206, 834, 207, 208, 209, 210, 835, 836, 1334, 3901, 3312, 2649, 1450, 4386, 3028, 1446, 1522, 3490, 217, 219, 1612, 220, 221, 222, 839, 223, 840, 224, 225, 228, 229, 230, 231, 842, 232, 233, 234, 238, 847, 239, 850, 851, 853, 1536, 240, 241, 242, 243, 244, 245, 247, 250, 251, 254, 255, 857, 256, 258, 259, 859, 260, 861, 862, 864, 263, 2318, 865, 2326, 264, 1241, 266, 1477, 867, 268, 868, 269, 270, 869, 870, 271, 871, 872, 272, 873, 1252, 1591, 875, 274, 275, 276, 876, 877, 277, 278, 280, 1892, 1539, 881, 3601, 3877, 1460, 282, 1799, 284, 1626, 286, 1374, 1620, 1290, 1354, 887, 1956, 889, 292, 4324, 4138, 891, 296, 3903, 297, 3829, 3809, 4271, 3844, 1436, 300, 1617, 1961, 302, 304, 305, 2147, 1589, 4370, 307, 3646, 4059, 1431, 1331, 312, 1779, 313, 896, 1558, 1552, 318, 319, 322, 1958, 323, 324, 901, 1491, 328, 329, 4484, 907, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3414, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 4248, 1557, 3804, 1602, 4188, 1330, 4349, 3337, 3533, 2629, 3038, 2539, 3567, 3291, 3348, 3115, 3359, 2993, 3079, 3506, 2527, 2963, 2938, 2516, 3113, 2639, 3143, 3044, 2747, 3154, 3280, 3039, 2743, 2902, 3056, 2554, 2522, 3103, 3210, 3434, 2656, 4251, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 2869, 2610, 3298, 2775, 3108, 2603, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 2584, 2712, 3213, 3451, 2634, 3150, 2810, 3303, 2573, 3460, 2615, 3394, 2828, 3329, 4377, 4499, 3555, 2974, 3629, 3578, 3885, 2817, 3248, 2954, 3334, 3571, 3345, 3551, 3384, 3422, 2530, 2973, 4418, 4081, 3951, 1764, 1671, 1843, 3449, 3185, 3399, 2786, 3055, 3472, 3397, 2601, 3564, 3277, 3548, 3179, 3407, 3841, 3347, 3142, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3120, 3286, 2843, 3465, 3946, 2682, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 4269, 3487, 3489, 3019, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1464, 1886, 2666, 3246, 3130, 3560, 3236, 2730, 3572, 3689, 2968, 1457, 3450, 2577, 2567, 2532, 3415, 2934, 2984, 2568, 4147, 3002, 2986, 910, 2979, 2542, 1762, 4472, 1529, 4057, 1976, 4507, 1583, 1711, 2013, 1594, 4440, 344, 920, 921, 4498, 3728, 3637, 3570, 3429, 4091, 2870, 2995, 4338, 3205, 2880, 3261, 3968, 4291, 4204, 4071, 4034, 1742, 2820, 3912, 3287, 1218, 1190, 4145, 354, 1782, 927, 3610, 356, 4224, 1918, 1959, 1840, 3736, 1770, 929, 930, 931, 4155, 360, 361, 362, 364, 1279, 365, 1543, 366, 367, 4049, 1633, 1722, 369, 1495, 1566, 373, 1211, 1726, 1442, 1748, 4142, 1434, 1358, 1191, 3732, 4110, 4039, 4506, 4156, 4036, 4030, 4016, 3899, 1247, 1855, 3919, 1244, 4140, 3930, 4117, 4189, 4001, 3643, 3619, 1240, 1575, 3871, 4135, 4408, 3604, 1274, 4149, 3741, 1938, 3709, 3622, 3834, 1983, 1743, 3979, 4461, 4113, 3817, 4232, 4214, 3902, 1219, 1486, 1512, 1516, 4047, 4082, 4094, 1574, 1248, 4078, 1872, 1426, 3687, 4318, 4243, 3866, 3704, 4172, 1856, 3324, 2769, 2678, 1561, 3296, 377, 1395, 1584, 4093, 4453, 4210, 4009, 4264, 4339, 1738, 4474, 4013, 4266, 1771, 1759, 1922, 3860, 3832, 1349, 1381, 3663, 1185, 3932, 3858, 1198, 4192, 3611, 1919, 3790, 3712, 4095, 1887, 4468, 1720, 4247, 934, 1790, 2002, 1657, 1336, 3894, 3576, 4314, 3695, 1850, 4362, 1815, 3638, 4454, 1661, 4504, 1359, 3650, 1305, 1648, 3831, 4435, 4451, 1993, 3849, 1718, 4068, 1835, 1425, 1428, 4380, 1658, 1452, 4024, 4072, 3791, 4491, 4323, 4014, 1304, 1511, 1981, 1676, 4422, 1283, 3861, 3644, 4259, 1418, 1176, 4216, 1421, 3992, 4423, 4253, 3748, 4119, 1297, 1954, 4450, 3711, 4344, 1578, 4384, 3892, 3674, 3595, 4405, 3973, 4109, 4159, 3744, 4387, 4275, 4492, 4365, 4118, 4278, 3751, 3678, 4353, 3175, 2970, 1228, 3534, 3437, 3406, 3641, 4470, 3941, 4348, 3587, 3818, 4350, 3821, 3929, 3824, 4465, 1398, 3828, 1284, 3773, 4209, 3014, 3884, 2783, 4038, 3545, 2916, 3112, 2834, 3880, 4399, 4048, 3752, 4207, 1235, 3943, 4355, 4087, 4100, 4163, 4486, 4178, 1643, 4152, 4445, 4303, 4284, 1213, 4382, 1399, 3714, 4487, 3615, 4018, 4420, 4121, 3724, 4282, 4310, 4488, 4127, 3670, 4052, 3627, 3777, 1371, 3685, 4258, 3812, 1581, 4124, 4508, 4500, 3652, 3972, 1528, 3717, 2740, 3125, 3546, 3842, 4404, 3690, 3962, 4136, 3589, 3907, 4153, 2707, 4180, 3923, 4444, 4361, 4394, 3758, 4510, 3742, 1710, 1407, 4187, 1193, 1188, 1196, 1724, 1717, 1200, 1997, 1701, 4280, 4006, 1984, 3833, 2561, 4442, 3900, 4298, 1761, 4388, 1597, 4317, 4249, 1341, 1203, 1680, 4441, 3628, 3725, 1945, 937, 4206, 1588, 1439, 3794, 4447, 3776, 3658, 4064, 4011, 4272, 4503, 1324, 3594, 3680, 1300, 1750, 3913, 3774, 1877, 1987, 3998, 3686, 3696, 4463, 3853, 4244, 4083, 1909, 3897, 1546, 3613, 3273, 3452, 3525, 1229, 3835, 1234, 3970, 3856, 3586, 1175, 4321, 2778, 4412, 4407, 3823, 1427, 1507, 1878, 4333, 3146, 4281, 4211, 3843, 1677, 1524, 1525, 1974, 1239, 4167, 3740, 1342, 1471, 4176, 1232, 1989, 4125, 1741, 1580, 4060, 1576, 1212, 3921, 1174, 4061, 4021, 3693, 3734, 1382, 3684, 3806, 3635, 3976, 3840, 4286, 3836, 4161, 3659, 3605, 4392, 4328, 1316, 4173, 4265, 1246, 1629, 1829, 3826, 1992, 1656, 1914, 1839, 3984, 3910, 4160, 4184, 4132, 4250, 1792, 3769, 1534, 4003, 1689, 1599, 1265, 1410, 1823, 1498, 1329, 386, 940, 1655, 3874, 4261, 3626, 3579, 1644, 3936, 4054, 4260, 4297, 4357, 4022, 4046, 1222, 1996, 3788, 3953, 4226, 4277, 4101, 3797, 4019, 3771, 3632, 1402, 4137, 3772, 3661, 4186, 4174, 1170, 3850, 1315, 1611, 1859, 4183, 1403, 4033, 3623, 3596, 1461, 1848, 1173, 1928, 1765, 3719, 3584, 4165, 4191, 3980, 4058, 4200, 4336, 3958, 1260, 4051, 3798, 4032, 1774, 4322, 3991, 4106, 3978, 3965, 4464, 3813, 4154, 1365, 3846, 4345, 4379, 3603, 3937, 3801, 1787, 1214, 4483, 4201, 1845, 1220, 4245, 1347, 3867, 1900, 3640, 3916, 4089, 3745, 3975, 4256, 4308, 4000, 3868, 4403, 3883, 4326, 4393, 3895, 4237, 3955, 4005, 4092, 4372, 4283, 3805, 4151, 3982, 3597, 3872, 3987, 4490, 3876, 3668, 4300, 1679, 1242, 3598, 3583, 3647, 4304, 3779, 1907, 4042, 3648, 3996, 3800, 4367, 4099, 1807, 3887, 4428, 1625, 1390, 1884, 1682, 4364, 4306, 3869, 1278, 942, 1505, 1709, 3600, 3914, 3948, 1865, 1716, 3961, 1506, 4221, 1376, 1683, 4276, 1721, 1632, 3607, 1455, 1913, 1497, 1317, 1951, 1778, 3768, 4225, 1668, 390, 4406, 4341, 1480, 1714, 1422, 397, 3618, 4433, 1177, 3898, 398, 950, 400, 1965, 4471, 953, 401, 403, 955, 1482, 3602, 4434, 3721, 1592, 1825, 1698, 1519, 1352, 4409, 1977, 405, 1631, 1684, 409, 3889, 2117, 411, 413, 414, 415, 961, 1905, 416, 963, 3675, 1833, 3974, 965, 970, 1897, 1272, 4133, 3830, 972, 424, 427, 1708, 4141, 3749, 1619, 975, 4044, 3590, 1944, 4126, 1923, 1706, 432, 976, 978, 4400, 1606, 980, 2604, 2895, 436, 3448, 2188, 438, 1692, 442, 3862, 4055, 4358, 2276, 985, 1664, 443, 4309, 4045, 3854, 3682, 1972, 2688, 4366, 3425, 3231, 3649, 2759, 2724, 3755, 1216, 3599, 3775, 2754, 2606, 3471, 3234, 2565, 2803, 2562, 2684, 3411, 3405, 2690, 1255, 3694, 3985, 3535, 1306, 4084, 3282, 1780, 448, 990, 991, 1485, 992, 1728, 993, 994, 1696, 997, 3116, 1869, 456, 1386, 1000, 3879, 2698, 460, 1001, 461, 1447, 1002, 1003, 463, 464, 4425, 465, 1004, 466, 469, 1231, 1468, 470, 1005, 476, 1273, 1744, 1488, 1659, 1604, 479, 1013, 1014, 481, 4313, 1499, 1016, 4073, 1777, 485, 4274, 487, 1775, 4205, 1526, 1697, 2000, 488, 1894, 1882, 1817, 1880, 490, 1018, 3917, 3616, 4311, 4473, 4085, 4401, 3783, 3765, 1538, 3870, 491, 3699, 3792, 4290, 494, 2421, 4090, 2012, 1803, 3642, 1322, 1020, 502, 1333, 504, 1890, 507, 3608, 1732, 1864, 1816, 1531, 513, 3636, 514, 1699, 1027, 1366, 4950, 4027, 3478, 2959, 1293, 3197, 1595, 3413, 518, 1032, 1033, 1675, 1797, 2315, 521, 1999, 3759, 1215, 4122, 1730, 3966, 1035, 4973, 528, 3911, 3289, 1821, 530, 532, 1874, 533, 1038, 538, 539, 3767, 1929, 3683, 1338, 1616, 1901, 1796, 1517, 4331, 4332, 4466, 2719, 4123, 4233, 1824, 3129, 3087, 3505, 4158, 4254, 3720, 4351, 4414, 3453, 3254, 1649, 544, 4240, 3580, 1046, 548, 549, 2598, 552, 553, 3819, 1259, 4062, 554, 2841, 3515, 1433, 1323, 4301, 555, 558, 3738, 1567, 559, 1054, 1270, 3857, 2175, 1257, 1238, 3662, 561, 1058, 563, 1061, 4203, 1899, 3981, 1062, 1303, 4116, 1946, 3620, 1514, 4002, 3679, 3964, 3707, 3666, 1348, 4273, 1804, 4497, 1401, 4438, 1895, 1264, 1912, 573, 3939, 4170, 4056, 1271, 1521, 1221, 1451, 3934, 4080, 4352, 1700, 3739, 1419, 1811, 3950, 1690, 3716, 1569, 1437, 1674, 4337, 1285, 4268, 1183, 3881, 3938, 4198, 4416, 4229, 4292, 4193, 4325, 4375, 4432, 4215, 3944, 4195, 4223, 1540, 4455, 4512, 1723, 3750, 4070, 4104, 4235, 1863, 4346, 1881, 1814, 4443, 3761, 1587, 3592, 576, 4469, 1275, 1950, 4129, 1296, 1551, 3733, 4111, 3993, 4108, 1287, 1713, 1556, 1736, 1995, 1705, 1861, 4120, 4417, 1785, 1236, 4293, 1827, 3839, 3983, 4395, 1957, 3928, 4295, 3715, 3855, 3609, 4076, 4175, 3927, 1757, 4299, 1773, 1364, 1462, 4007, 3729, 3665, 3722, 1691, 1712, 1624, 1751, 1394, 3924, 3952, 581, 2003, 1559, 1250, 1555, 4114, 1818, 1076, 4448, 1078, 1786, 3354, 1832, 3432, 2891, 1903, 4459, 589, 1563, 1084, 591, 592, 594, 595, 1858, 596, 1453, 2559, 2590, 2738, 4478, 4069, 1809, 1327, 4150, 3260, 3481, 3672, 3625, 4439, 3852, 3997, 3847, 4050, 3377, 4424, 4143, 4493, 3492, 1412, 4017, 4477, 1794, 602, 4263, 3072, 2009, 1754, 3888, 603, 604, 1368, 3827, 4103, 1571, 1438, 611, 1093, 612, 1094, 3701, 4389, 614, 615, 1332, 1715, 4369, 1652, 1541, 1921, 1733, 1373, 1870, 619, 4218, 1798, 1702, 1384, 3700, 1788, 1600, 624, 2473, 1423, 2771, 1339, 3875, 3070, 2015, 2557, 2795, 2906, 2103, 3299, 3118, 4576, 1103, 1614, 1947, 631, 4479, 3766, 1107, 635, 1813, 636, 2431, 637, 1109, 639, 3706, 1110, 640, 642, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 1269, 3095, 1286, 3624, 4481, 3511, 2735, 4381, 3295, 1568, 1766, 3221, 2643, 2739, 3920, 647, 1801, 4279, 3762, 1637, 1509, 648, 1117, 1119, 2965, 2695, 3293, 4722, 1121, 653, 1122, 3264, 655, 656, 657, 4035, 2910, 3391, 2808, 3099, 2913, 3676, 2852, 3284, 2731, 3537, 3994, 1319, 4467, 1623, 660, 1123, 664, 1126, 1128, 1129, 1130, 670, 1527, 671, 1772, 1137, 5005, 1933, 1731, 1793, 3342, 1140, 1141, 687, 3822, 1601, 1148, 693, 3905, 694, 1414, 1149, 697, 1795, 1372, 1695, 1866, 1151, 4115, 1719, 1920, 4999, 1544, 703, 4031, 4305, 1636, 3810, 4452, 3677, 4227, 4402, 4157, 4197, 1337, 1253, 4028, 3989, 704, 1493, 1902, 1152, 4182, 707, 708, 709, 4476, 1375, 713, 714, 1841, 4234, 1154, 1314, 4096, 718, 719, 720, 2063, 1311, 721, 4217, 1627, 1158, 1953, 1201, 3787, 729, 3747, 730, 1846, 3815, 1964, 1822, 1262, 4480, 1537, 1291, 1318, 4294, 1582, 3439, 2833, 4287, 4509, 4505, 4267, 3697, 3272, 4112, 2967, 3651, 1805, 3705, 1195, 3859, 1164, 1165, 2351, 2423, 1745, 1973, 3807, 4307, 1169, 1454, or 1781.

In some aspects, (a) X³ of SEQ ID NO: 5014 is not D, (b) X⁶ of SEQ ID NO: 5014 is not D, or (c) X³ of SEQ ID NO: 5014 is not D and X⁶ of SEQ ID NO: 5014 is not D. In some aspects, the 581 to 589 region of the VP capsid polypeptide has a sequence of any one of SEQ ID NOs: 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 2469, 4889, 21, 3192, 4942, 22, 23, 1638, 748, 24, 25, 749, 26, 27, 1703, 1836, 28, 2038, 2301, 3660, 29, 750, 30, 31, 32, 2095, 1640, 33, 34, 35, 36, 37, 4742, 2450, 38, 4925, 39, 751, 752, 2144, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2080, 2072, 2034, 50, 1308, 1230, 3960, 1378, 2994, 3080, 2589, 3681, 51, 2167, 52, 760, 53, 2389, 4755, 761, 4591, 762, 4659, 54, 2146, 3925, 55, 3654, 2087, 2346, 4766, 4682, 4984, 4744, 56, 763, 764, 4626, 4702, 57, 58, 59, 60, 61, 4086, 1740, 1660, 765, 766, 62, 1966, 2032, 2499, 2332, 767, 63, 2096, 2181, 4913, 2438, 2041, 64, 768, 769, 770, 2384, 2402, 771, 65, 66, 2111, 67, 772, 2458, 2197, 68, 3816, 69, 2599, 2706, 2497, 70, 2399, 71, 4769, 2366, 72, 73, 74, 3865, 1549, 1361, 2453, 3882, 773, 3713, 1942, 2140, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2379, 81, 82, 4065, 83, 84, 2030, 1294, 2413, 2064, 1484, 2299, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 93, 2356, 784, 785, 786, 94, 95, 788, 1831, 1867, 96, 4458, 97, 1970, 4656, 2173, 1936, 2349, 1590, 98, 99, 100, 789, 790, 1298, 101, 1688, 791, 792, 2007, 4556, 2070, 102, 793, 4632, 103, 794, 795, 104, 105, 106, 2229, 796, 4980, 4691, 107, 2440, 1849, 108, 2014, 797, 4736, 2509, 2018, 2227, 4787, 1579, 110, 1906, 111, 2428, 798, 2075, 112, 2411, 113, 2359, 799, 4848, 800, 2461, 1444, 2313, 2350, 4579, 4821, 801, 114, 2329, 4864, 4834, 115, 1344, 116, 802, 117, 118, 119, 4929, 4677, 120, 4791, 2158, 121, 803, 804, 1463, 805, 122, 2139, 2290, 1760, 2465, 123, 2424, 2058, 2230, 124, 4717, 806, 125, 126, 2370, 127, 4521, 128, 4971, 4994, 807, 2085, 4533, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 4544, 5009, 144, 145, 816, 4654, 2125, 4663, 4810, 4816, 1634, 1302, 817, 146, 818, 147, 148, 149, 4546, 150, 151, 152, 153, 154, 155, 819, 820, 156, 2149, 157, 2295, 821, 158, 159, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 2170, 4809, 4520, 180, 826, 827, 181, 828, 4695, 2130, 4911, 182, 4582, 4873, 2189, 183, 4641, 829, 184, 185, 2244, 4647, 186, 187, 188, 189, 190, 2114, 830, 191, 192, 193, 194, 2071, 4615, 2361, 195, 4852, 2187, 4914, 4947, 196, 1391, 197, 4534, 4726, 4788, 198, 199, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2090, 2241, 217, 218, 4653, 219, 1612, 220, 221, 222, 839, 223, 4974, 4790, 4915, 2464, 4567, 4774, 4964, 2360, 840, 224, 4928, 4741, 225, 2088, 4751, 226, 227, 4890, 4519, 4883, 228, 4786, 229, 230, 231, 2393, 2448, 841, 842, 232, 233, 4782, 234, 2269, 843, 235, 844, 236, 237, 4930, 845, 846, 238, 847, 239, 848, 849, 2150, 850, 851, 852, 853, 1536, 240, 854, 241, 242, 243, 244, 245, 246, 247, 248, 4696, 855, 4634, 856, 249, 250, 251, 252, 253, 254, 255, 857, 2115, 4880, 2218, 256, 257, 258, 259, 858, 859, 260, 261, 860, 861, 862, 262, 4966, 863, 864, 263, 4749, 4970, 2296, 2318, 4535, 865, 2326, 264, 1241, 265, 266, 866, 1477, 267, 867, 4977, 268, 868, 269, 4668, 5002, 270, 869, 870, 271, 871, 872, 272, 4822, 4709, 873, 4655, 4972, 2392, 2317, 874, 1252, 273, 1591, 4975, 875, 274, 2025, 275, 1980, 276, 876, 877, 2066, 277, 278, 279, 4670, 280, 2323, 2288, 2334, 4939, 4689, 281, 878, 2416, 1892, 879, 1539, 2214, 2215, 880, 2327, 881, 882, 4601, 883, 1295, 3601, 3877, 1930, 1420, 1678, 1460, 2306, 884, 282, 1799, 283, 284, 4988, 4871, 4553, 4772, 4817, 2260, 1626, 2082, 885, 286, 1374, 886, 2119, 2504, 2468, 287, 1620, 1290, 2425, 2482, 4564, 4671, 2282, 288, 2354, 1354, 2033, 289, 887, 290, 888, 291, 1956, 2362, 2467, 889, 2357, 4547, 4765, 292, 4794, 2391, 4324, 293, 890, 294, 295, 4138, 891, 2297, 2254, 296, 2224, 3903, 297, 892, 3829, 298, 299, 3809, 4271, 3844, 1436, 300, 1515, 1617, 301, 1961, 4649, 5010, 302, 2060, 303, 304, 305, 2452, 2147, 4574, 4538, 306, 2293, 1589, 893, 4946, 4370, 4841, 2342, 307, 308, 309, 894, 2246, 2155, 3646, 4059, 1431, 1331, 310, 311, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 904, 1635, 4820, 905, 2506, 331, 4706, 4484, 1756, 906, 907, 4661, 2179, 4584, 4609, 332, 4907, 4635, 4529, 4683, 4523, 4561, 333, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4319, 3906, 1533, 1755, 1888, 4248, 1557, 2153, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 2363, 908, 1935, 4711, 4728, 2460, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 4943, 3002, 2986, 910, 2979, 2542, 2338, 2331, 2492, 1762, 4472, 1472, 1529, 2319, 2131, 4057, 4937, 2048, 4887, 1976, 4507, 1583, 338, 2078, 2372, 2102, 339, 911, 1711, 340, 912, 2013, 913, 2277, 914, 2403, 341, 915, 4672, 4902, 916, 342, 1594, 4440, 917, 343, 344, 4559, 918, 2439, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 371, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2345, 4471, 952, 953, 401, 402, 2475, 403, 404, 4758, 954, 955, 2437, 4139, 1482, 3602, 4434, 3721, 1592, 1825, 1698, 1519, 1352, 4202, 4409, 1977, 1917, 405, 406, 407, 408, 956, 957, 2267, 1631, 1684, 409, 2054, 3889, 958, 410, 2117, 411, 959, 412, 960, 2443, 4712, 4619, 2285, 413, 414, 415, 961, 2352, 2417, 962, 1905, 416, 963, 417, 418, 2382, 3675, 1833, 3974, 419, 964, 420, 421, 965, 966, 967, 4919, 968, 2365, 969, 970, 422, 1897, 2056, 2309, 1272, 4754, 2207, 971, 423, 4133, 3830, 4865, 972, 424, 973, 425, 2068, 4955, 426, 974, 2109, 2380, 427, 1708, 4676, 428, 4141, 429, 430, 3749, 431, 1619, 975, 4044, 3590, 1944, 4126, 1923, 1706, 432, 976, 977, 978, 4867, 4400, 433, 434, 1606, 2143, 979, 435, 980, 2604, 2895, 981, 436, 3448, 4959, 4563, 2188, 437, 438, 2055, 1692, 982, 2388, 2243, 983, 2035, 2120, 4940, 439, 440, 441, 2474, 4900, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 2242, 453, 2286, 992, 1728, 993, 4703, 994, 995, 454, 1696, 996, 2039, 455, 997, 4874, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 466, 2328, 467, 468, 469, 1231, 1468, 470, 471, 2496, 472, 473, 1005, 474, 475, 2108, 4725, 1006, 476, 1273, 1007, 1744, 1488, 1659, 477, 478, 4598, 1604, 4753, 2493, 479, 1008, 1009, 480, 1010, 1011, 1012, 1013, 1014, 481, 4313, 482, 483, 1499, 484, 1015, 4761, 2091, 2386, 2513, 1016, 4073, 1777, 2172, 485, 2205, 4592, 4545, 4720, 486, 1458, 4274, 2122, 4905, 4796, 2152, 4539, 4528, 4904, 4842, 4986, 4639, 4846, 4623, 4650, 4595, 2195, 4681, 487, 2182, 4710, 1775, 2083, 4205, 1526, 4586, 1697, 1017, 2000, 4692, 4795, 2480, 2017, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 5001, 2136, 493, 2211, 4532, 494, 2421, 495, 4090, 2378, 2104, 2210, 2247, 2478, 2047, 496, 4620, 4978, 4614, 1019, 2012, 1803, 3642, 497, 1322, 4752, 1020, 498, 1021, 2235, 2302, 2488, 4665, 4699, 4608, 4781, 499, 2049, 2404, 4685, 500, 501, 1022, 502, 1333, 503, 1023, 4686, 4804, 504, 4727, 505, 506, 4938, 4805, 2065, 1890, 4891, 1024, 507, 508, 509, 2110, 1025, 510, 1026, 3608, 1732, 511, 2397, 1864, 1816, 1531, 512, 513, 2451, 3636, 5012, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 2093, 4746, 2489, 4922, 2145, 1030, 4832, 4950, 1261, 4027, 3478, 3763, 2959, 1293, 2462, 517, 3197, 1595, 3413, 2067, 1031, 2228, 1448, 518, 4698, 4616, 2057, 2305, 2133, 2061, 4526, 4617, 1032, 1033, 2429, 2311, 1675, 1797, 519, 4578, 2271, 4693, 4604, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 535, 1038, 536, 1039, 537, 538, 539, 1040, 3767, 1929, 1041, 1042, 3683, 2100, 4853, 2300, 2165, 540, 2105, 541, 1043, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1649, 543, 544, 545, 546, 1045, 547, 2225, 4240, 3580, 4680, 1046, 548, 549, 1047, 550, 2598, 551, 552, 553, 3819, 1259, 4062, 1048, 554, 2841, 1049, 1050, 3515, 2219, 1433, 1323, 4301, 555, 556, 1051, 557, 558, 1052, 1053, 3738, 1567, 559, 1054, 1270, 3857, 1055, 2151, 560, 2175, 2089, 1056, 4514, 2198, 2193, 1257, 1238, 3662, 561, 2186, 1057, 4637, 562, 1058, 563, 4872, 2097, 2436, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 575, 4443, 3761, 1346, 1587, 1072, 3592, 576, 1891, 4469, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2185, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 2304, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2113, 590, 591, 1085, 2256, 592, 2308, 593, 594, 2274, 595, 4931, 2407, 1858, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 2321, 1086, 601, 4017, 4477, 1794, 2483, 4776, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 2320, 2069, 610, 611, 1093, 612, 1094, 3701, 4389, 613, 4897, 1603, 614, 1095, 2154, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4855, 4847, 2398, 2239, 1101, 1667, 1384, 623, 4745, 3700, 1788, 1600, 624, 2098, 625, 4550, 4721, 4960, 4878, 2473, 4792, 4675, 1102, 1423, 626, 2771, 627, 1339, 3875, 628, 629, 3070, 4565, 2118, 2015, 4818, 4860, 2557, 2795, 2906, 2103, 4869, 2029, 3299, 3118, 4932, 4838, 4901, 4908, 4996, 4779, 2377, 4657, 4646, 4875, 4627, 4515, 4876, 4965, 4605, 4576, 2028, 4985, 4799, 4850, 1103, 4757, 4747, 1614, 1947, 2237, 630, 631, 4997, 4618, 4479, 3766, 632, 1104, 1105, 4886, 1106, 2432, 4827, 633, 1107, 4530, 634, 2371, 1108, 4536, 635, 1813, 636, 2431, 4866, 4991, 4814, 4560, 4956, 637, 4555, 1109, 4941, 4633, 2213, 638, 639, 3706, 1110, 640, 1111, 641, 4581, 4893, 4831, 2481, 4644, 4734, 2190, 4524, 4881, 4837, 4888, 4648, 642, 4548, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4629, 1115, 4918, 4688, 3920, 644, 4723, 4949, 4748, 4607, 4719, 1116, 645, 646, 4611, 2353, 4603, 4762, 4819, 2221, 4636, 4992, 4998, 2307, 647, 1801, 4279, 3762, 1637, 1509, 4843, 4797, 4590, 4516, 4995, 4541, 2200, 4580, 4858, 648, 649, 2502, 2477, 4724, 4551, 4674, 1118, 1119, 2965, 2369, 2695, 3293, 650, 651, 4730, 4862, 4722, 4944, 1120, 652, 1121, 4983, 4784, 4877, 4643, 653, 654, 4909, 1122, 3264, 655, 2076, 4849, 656, 657, 2447, 658, 659, 2191, 4035, 2910, 3391, 2808, 3099, 2913, 3676, 2852, 3284, 2731, 1609, 2375, 2201, 3537, 3994, 1319, 4467, 1802, 1227, 1623, 660, 4844, 4714, 4979, 4812, 2192, 1123, 661, 2364, 662, 663, 664, 1124, 1125, 665, 2487, 4899, 2138, 1126, 666, 667, 4569, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 674, 2456, 4588, 4963, 4851, 4558, 4557, 4697, 4610, 4773, 4687, 4708, 4910, 2123, 1136, 1137, 2358, 5005, 2208, 4952, 4945, 1933, 1731, 1793, 675, 676, 3342, 1138, 1139, 677, 4840, 4597, 4870, 1140, 4990, 4954, 4916, 678, 1141, 4737, 4527, 2253, 4531, 679, 1142, 1143, 680, 4771, 1144, 4638, 681, 4705, 4525, 4522, 4898, 2325, 2415, 5004, 1145, 682, 683, 2141, 1146, 684, 685, 2445, 686, 687, 4861, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4690, 4859, 4793, 4575, 2160, 4828, 4731, 4718, 4802, 4750, 1150, 2081, 2258, 697, 1795, 1372, 1695, 1866, 1151, 4115, 1719, 2280, 4662, 698, 2176, 2217, 1920, 699, 700, 701, 4999, 1544, 4739, 2512, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 2340, 4182, 706, 4652, 2442, 2484, 707, 708, 709, 4667, 1153, 2412, 4732, 4476, 2053, 710, 1375, 711, 712, 713, 4829, 4738, 4811, 4570, 1245, 714, 2045, 4707, 715, 2169, 4713, 1841, 4234, 4924, 1154, 1314, 4096, 2023, 717, 4895, 2414, 718, 1155, 719, 720, 2063, 1311, 4921, 721, 4833, 4903, 4885, 4666, 2249, 4217, 1156, 723, 1157, 1627, 2116, 4566, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 2248, 730, 731, 1846, 2507, 2368, 1162, 2019, 2333, 3815, 2005, 2266, 1964, 2347, 732, 2177, 2059, 4958, 4927, 1822, 4815, 1262, 2094, 4480, 1537, 1291, 2419, 1318, 4628, 4294, 2486, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4679, 4835, 2259, 4839, 2275, 1166, 4968, 4912, 2423, 737, 1167, 4989, 4684, 4612, 2127, 1745, 1973, 4767, 1168, 2273, 2310, 3807, 4307, 1169, 2303, 1454, or 1781. In some aspects, the 581 to 589 region of the VP capsid polypeptide has a sequence of any one of SEQ ID NOs: 2498, 4884, 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 2469, 4889, 21, 3192, 4942, 22, 23, 1638, 748, 24, 25, 749, 26, 27, 1703, 1836, 28, 2038, 2301, 3660, 29, 750, 30, 31, 2095, 1640, 33, 34, 35, 36, 37, 4742, 2450, 38, 4925, 39, 751, 752, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2072, 2034, 50, 1308, 1230, 3960, 1378, 2994, 3080, 2589, 3681, 51, 4694, 2257, 2167, 52, 760, 53, 2389, 4934, 4951, 4755, 761, 4591, 762, 4659, 54, 2146, 3925, 55, 3654, 2087, 2346, 4766, 4682, 4984, 4744, 56, 763, 764, 4626, 4702, 57, 58, 59, 60, 61, 4086, 1740, 1660, 765, 766, 62, 1966, 2032, 2499, 2332, 767, 63, 2096, 2181, 4913, 2438, 2041, 64, 768, 769, 770, 2384, 2402, 771, 65, 66, 67, 772, 2458, 2197, 68, 3816, 69, 2599, 2706, 2497, 70, 2234, 2183, 2399, 71, 4769, 2366, 72, 73, 74, 3865, 1549, 1361, 2453, 3882, 773, 3713, 1942, 2140, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2132, 81, 82, 4065, 83, 84, 2030, 1294, 2413, 2064, 1484, 2299, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 2356, 784, 785, 786, 94, 95, 787, 788, 1831, 1867, 96, 4458, 97, 1970, 4656, 2173, 1936, 2349, 1590, 4585, 98, 99, 100, 789, 790, 1298, 101, 1688, 791, 792, 2007, 4556, 2070, 102, 793, 4632, 103, 794, 795, 104, 105, 106, 2229, 796, 4691, 107, 2440, 1849, 108, 797, 4736, 2509, 2018, 2227, 4787, 109, 1579, 110, 1906, 111, 2428, 798, 2075, 112, 2411, 113, 2359, 799, 4848, 800, 2461, 1444, 2313, 2350, 4579, 4821, 801, 114, 2329, 4864, 4834, 115, 1344, 116, 802, 117, 118, 119, 4929, 4677, 120, 4791, 2158, 121, 803, 804, 1463, 805, 122, 2139, 2290, 1760, 2465, 123, 2424, 2058, 2230, 124, 4717, 806, 125, 126, 2370, 127, 4521, 128, 4971, 4994, 807, 4533, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 5009, 144, 145, 816, 4654, 2125, 4663, 4810, 4816, 1634, 1302, 817, 146, 818, 147, 148, 149, 4546, 150, 151, 152, 153, 154, 155, 819, 820, 156, 2149, 157, 2295, 821, 158, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 4809, 4520, 180, 826, 827, 181, 828, 4695, 2130, 4911, 182, 4582, 4873, 4596, 2020, 2189, 183, 4641, 829, 184, 185, 2244, 4647, 186, 187, 188, 189, 190, 830, 191, 192, 193, 194, 2071, 4615, 2361, 195, 4852, 4914, 4947, 196, 1391, 197, 4534, 4726, 198, 199, 4920, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2090, 2241, 217, 218, 4653, 219, 1612, 220, 221, 222, 839, 223, 4953, 4974, 4790, 4915, 2464, 4567, 4774, 4964, 2360, 840, 224, 4928, 4741, 225, 2088, 4751, 5007, 4542, 226, 227, 4890, 4519, 4883, 228, 4786, 229, 230, 231, 2448, 841, 842, 232, 233, 4782, 234, 4962, 2269, 843, 235, 844, 236, 237, 4930, 845, 846, 238, 847, 4704, 239, 848, 849, 2150, 850, 851, 852, 853, 1536, 240, 854, 241, 242, 243, 244, 245, 246, 247, 248, 4923, 2245, 4696, 855, 4634, 856, 249, 250, 251, 252, 253, 254, 255, 857, 2115, 2218, 256, 257, 258, 259, 858, 859, 260, 261, 860, 861, 862, 262, 4966, 863, 864, 263, 4749, 4970, 2296, 2318, 4535, 865, 2326, 264, 1241, 265, 266, 866, 1477, 267, 867, 268, 868, 269, 4668, 270, 869, 870, 271, 871, 872, 272, 4822, 4709, 873, 4655, 4972, 2317, 874, 1252, 273, 1591, 4975, 875, 274, 2025, 275, 1980, 276, 876, 877, 2066, 277, 278, 279, 4670, 280, 2323, 2288, 2334, 4939, 4689, 281, 878, 2416, 1892, 879, 1539, 2214, 2215, 880, 2327, 881, 882, 4601, 1295, 3601, 3877, 1930, 1420, 1678, 1460, 2306, 884, 282, 1799, 283, 284, 4988, 4871, 4553, 4772, 2260, 285, 1626, 885, 286, 1374, 886, 2119, 2504, 2468, 287, 2408, 1620, 1290, 2425, 2482, 4564, 4671, 2282, 288, 1354, 2033, 289, 887, 290, 888, 291, 1956, 2362, 2467, 889, 2357, 4547, 4765, 292, 4794, 2391, 4324, 293, 890, 294, 295, 4138, 891, 2297, 2254, 296, 2224, 3903, 297, 892, 3829, 298, 299, 3809, 4271, 3844, 1436, 300, 1515, 1617, 4701, 2174, 301, 1961, 4649, 5010, 302, 2060, 303, 304, 305, 2452, 2147, 4574, 4538, 306, 2293, 1589, 893, 4946, 4370, 4841, 2342, 307, 308, 309, 894, 2246, 2155, 3646, 4059, 1431, 1331, 311, 4836, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 1635, 4820, 905, 331, 4706, 2092, 4484, 1756, 906, 907, 4661, 2179, 4584, 4609, 332, 4907, 4635, 4529, 4683, 4561, 333, 2337, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4783, 4319, 3906, 1533, 1755, 1888, 4248, 1557, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 2444, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 2363, 908, 1935, 4711, 4728, 2460, 4517, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 337, 1184, 3450, 2577, 2567, 2532, 3415, 2934, 2984, 2568, 4147, 4943, 3002, 2986, 910, 2979, 2542, 2338, 2331, 2492, 1762, 4472, 1472, 1529, 2319, 4057, 4937, 2048, 4887, 1976, 4507, 1583, 338, 2078, 2372, 2102, 339, 911, 1711, 340, 912, 2013, 913, 2277, 914, 2403, 341, 915, 4902, 916, 342, 1594, 4440, 917, 343, 344, 4559, 918, 2233, 2439, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 2471, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 371, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2343, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 2383, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2129, 2345, 4471, 952, 953, 401, 2475, 403, 404, 4758, 954, 955, 2437, 4139, 1482, 3602, 4434, 3721, 1592, 1825, 1698, 1519, 1352, 4202, 4409, 1977, 1917, 405, 406, 407, 408, 956, 2272, 957, 2267, 1631, 1684, 409, 2054, 3889, 958, 410, 2117, 411, 959, 412, 960, 2443, 4712, 4619, 2285, 413, 414, 415, 961, 2352, 2417, 962, 1905, 416, 963, 417, 418, 2382, 3675, 1833, 3974, 419, 964, 420, 421, 965, 966, 967, 968, 2365, 969, 970, 422, 1897, 2056, 2309, 4733, 1272, 4754, 2207, 971, 423, 4133, 3830, 4865, 972, 424, 973, 425, 4955, 426, 974, 2109, 2380, 427, 1708, 4676, 428, 4141, 429, 430, 3749, 431, 1619, 975, 4044, 3590, 1944, 4126, 1923, 1706, 432, 976, 977, 978, 4867, 4400, 433, 434, 1606, 979, 435, 980, 2604, 2895, 981, 436, 3448, 4959, 4563, 2188, 437, 438, 2055, 1692, 982, 2388, 2433, 2243, 983, 2035, 2120, 4940, 439, 440, 441, 2474, 442, 4900, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 2242, 453, 2286, 992, 1728, 993, 4703, 994, 995, 454, 1696, 996, 2039, 997, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 466, 2328, 467, 468, 469, 1231, 1468, 470, 471, 2496, 472, 473, 1005, 474, 2108, 4725, 1006, 476, 1273, 1007, 1744, 1488, 1659, 477, 478, 4598, 1604, 4753, 2493, 479, 1008, 1009, 1010, 1011, 1012, 1013, 1014, 481, 4313, 482, 483, 1499, 484, 1015, 4761, 2091, 2386, 2513, 1016, 4073, 1777, 2172, 485, 2205, 4592, 4545, 4720, 486, 1458, 4274, 2122, 4905, 5003, 4599, 4796, 2152, 4539, 4528, 4904, 4842, 4986, 4639, 4846, 4623, 4650, 4595, 2195, 4681, 2142, 487, 2182, 4710, 1775, 2083, 4205, 1526, 4586, 4602, 4518, 1697, 1017, 2000, 4692, 2480, 2017, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 2202, 5001, 2136, 493, 2211, 4532, 494, 2421, 495, 4090, 2378, 2210, 2247, 2478, 2047, 496, 4620, 4978, 1019, 2012, 1803, 3642, 497, 1322, 4752, 1020, 498, 1021, 2235, 2302, 2488, 4665, 4540, 5006, 4699, 4608, 4781, 499, 2049, 2404, 4685, 500, 501, 1022, 502, 1333, 503, 1023, 4686, 4804, 504, 4727, 505, 506, 4805, 2065, 1890, 1024, 507, 508, 509, 2110, 1025, 510, 1026, 3608, 1732, 511, 2397, 1864, 1816, 1531, 512, 513, 2451, 3636, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 4746, 2489, 4922, 2145, 1030, 4832, 4950, 1261, 4027, 3478, 3763, 2959, 1293, 2462, 517, 3197, 1595, 3413, 2238, 2067, 1031, 2228, 1448, 518, 4698, 4616, 2057, 2305, 2133, 2061, 4526, 4617, 1032, 1033, 2429, 2311, 1675, 4785, 1797, 519, 4578, 2271, 4693, 4604, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 1038, 536, 1039, 537, 538, 539, 1040, 3767, 1929, 1041, 1042, 3683, 2100, 4853, 2300, 2165, 540, 2105, 541, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1649, 543, 544, 545, 546, 1045, 547, 2084, 2225, 4240, 3580, 4680, 1046, 548, 2062, 549, 1047, 550, 2598, 551, 552, 553, 3819, 1259, 4062, 1048, 554, 2841, 1049, 1050, 3515, 1433, 1323, 4301, 555, 556, 1051, 557, 558, 1052, 1053, 3738, 1567, 559, 1054, 1270, 3857, 1055, 2151, 560, 2175, 2089, 4514, 2198, 2193, 1257, 1238, 3662, 561, 2186, 1057, 562, 1058, 563, 4872, 2097, 2436, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1705, 1861, 1197, 1254, 4120, 1545, 4417, 1469, 1292, 2185, 1785, 1236, 4293, 1827, 3839, 3983, 4395, 1957, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 2304, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2255, 2113, 590, 591, 1085, 2256, 592, 2308, 593, 594, 595, 4931, 2407, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 2321, 1086, 601, 4017, 4477, 1794, 2483, 4776, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 2320, 2069, 610, 611, 1093, 612, 1094, 3701, 4389, 613, 4897, 1603, 614, 1095, 2154, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4801, 4855, 4847, 2398, 2239, 1101, 1667, 1384, 623, 4745, 3700, 1788, 1600, 624, 2098, 625, 4550, 4721, 4960, 2473, 4792, 4675, 1102, 1423, 626, 2771, 627, 1339, 3875, 628, 629, 3070, 4565, 2118, 2015, 4818, 4860, 2557, 2795, 2906, 2103, 4869, 2029, 3299, 3118, 4932, 4838, 4901, 4908, 4996, 4779, 2377, 4657, 4646, 4875, 4627, 4515, 4876, 4965, 4605, 4576, 2028, 4985, 4799, 4850, 1103, 4757, 4747, 1614, 1947, 2237, 630, 631, 4997, 4618, 4479, 3766, 632, 1104, 1105, 4886, 1106, 2432, 4827, 633, 1107, 4530, 634, 2371, 4651, 1108, 4536, 635, 1813, 636, 2431, 4866, 4991, 4814, 4560, 4956, 637, 4555, 1109, 4577, 4633, 2213, 638, 639, 3706, 1110, 640, 1111, 641, 4581, 4893, 4831, 2481, 4644, 4734, 2190, 4881, 4837, 4888, 4648, 642, 4548, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4760, 4629, 1115, 4918, 4688, 3920, 644, 4723, 4949, 4607, 4719, 1116, 645, 646, 4611, 2353, 4645, 4603, 4762, 4819, 2221, 4636, 4992, 2307, 647, 1801, 4279, 3762, 1637, 1509, 4843, 4797, 4590, 4516, 4541, 2200, 4580, 4858, 648, 649, 1117, 4780, 4800, 2477, 4724, 4551, 4674, 1118, 1119, 2965, 2369, 2695, 3293, 650, 651, 4862, 4722, 4944, 1120, 652, 1121, 4983, 4784, 4877, 653, 654, 1122, 3264, 655, 2076, 4849, 656, 657, 2447, 658, 659, 2191, 4035, 2910, 3391, 2808, 3099, 2913, 3676, 2852, 3284, 2731, 1609, 2375, 3537, 3994, 1319, 4467, 1802, 1227, 1623, 660, 4844, 2086, 4770, 4714, 4979, 4812, 2192, 1123, 661, 2364, 662, 663, 664, 1124, 1125, 665, 2487, 4899, 2138, 1126, 666, 667, 4569, 4554, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 674, 2456, 4606, 4588, 4963, 4851, 4558, 4557, 4697, 4610, 4773, 4687, 4708, 4910, 2123, 1136, 1137, 2358, 5005, 2208, 4952, 4945, 1933, 1731, 1793, 675, 676, 3342, 1138, 1139, 677, 4840, 4597, 4870, 1140, 4990, 4954, 4916, 678, 1141, 4737, 4527, 2253, 4531, 679, 1142, 1143, 680, 4771, 1144, 4638, 681, 4705, 4525, 4898, 2325, 2415, 5004, 1145, 682, 683, 2141, 1146, 684, 685, 2445, 686, 687, 4861, 2157, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4690, 4859, 4793, 4575, 2160, 4828, 4731, 4802, 4750, 1150, 2081, 2258, 697, 1795, 1372, 1695, 1866, 1151, 4115, 1719, 2280, 4662, 698, 2176, 2194, 2217, 1920, 699, 700, 701, 4999, 1544, 4739, 2512, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 4824, 705, 2340, 4182, 706, 4652, 2442, 2484, 707, 708, 709, 4667, 1153, 2412, 4732, 4476, 2053, 710, 1375, 711, 712, 713, 4738, 4811, 4570, 4961, 1245, 714, 2045, 4707, 715, 2169, 4713, 4879, 1841, 4234, 4924, 1154, 1314, 4096, 2023, 716, 717, 4895, 2414, 718, 1155, 719, 720, 2063, 1311, 4921, 721, 4642, 722, 4833, 4903, 4885, 4666, 2249, 4217, 1156, 723, 1157, 1627, 2116, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 2248, 730, 731, 2024, 1846, 2507, 2368, 1162, 2019, 2333, 3815, 2005, 2266, 1964, 2347, 2385, 732, 2177, 2059, 4958, 4927, 1822, 4815, 1262, 2094, 4480, 1537, 1291, 2419, 1318, 2283, 4976, 4628, 4294, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4835, 2259, 4571, 2275, 1166, 4968, 2250, 2423, 737, 1167, 4989, 4669, 4552, 4612, 2127, 1745, 1973, 4767, 1168, 2273, 2310, 3807, 4307, 1169, 2303, 1454, or 1781. In some aspects, the 581 to 589 region of the VP capsid polypeptide has a sequence of any one of SEQ ID NOs: 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 2469, 4889, 21, 3192, 4942, 22, 23, 1638, 748, 24, 25, 749, 26, 27, 1703, 1836, 28, 2038, 2301, 3660, 29, 750, 30, 31, 2095, 1640, 33, 34, 35, 36, 37, 4742, 2450, 38, 4925, 39, 751, 752, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2072, 2034, 50, 1308, 1230, 3960, 1378, 2994, 3080, 2589, 3681, 51, 2167, 52, 760, 53, 2389, 4755, 761, 4591, 762, 4659, 54, 2146, 3925, 55, 3654, 2087, 2346, 4766, 4682, 4984, 4744, 56, 763, 764, 4626, 4702, 57, 58, 59, 60, 61, 4086, 1740, 1660, 765, 766, 62, 1966, 2032, 2499, 2332, 767, 63, 2096, 2181, 4913, 2438, 2041, 64, 768, 769, 770, 2384, 2402, 771, 65, 66, 67, 772, 2458, 2197, 68, 3816, 69, 2599, 2706, 2497, 70, 2399, 71, 4769, 2366, 72, 73, 74, 3865, 1549, 1361, 2453, 3882, 773, 3713, 1942, 2140, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 81, 82, 4065, 83, 84, 2030, 1294, 2413, 2064, 1484, 2299, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 2356, 784, 785, 786, 94, 95, 788, 1831, 1867, 96, 4458, 97, 1970, 4656, 2173, 1936, 2349, 1590, 98, 99, 100, 789, 790, 1298, 101, 1688, 791, 792, 2007, 4556, 2070, 102, 793, 4632, 103, 794, 795, 104, 105, 106, 2229, 796, 4691, 107, 2440, 1849, 108, 797, 4736, 2509, 2018, 2227, 4787, 1579, 110, 1906, 111, 2428, 798, 2075, 112, 2411, 113, 2359, 799, 4848, 800, 2461, 1444, 2313, 2350, 4579, 4821, 801, 114, 2329, 4864, 4834, 115, 1344, 116, 802, 117, 118, 119, 4929, 4677, 120, 4791, 2158, 121, 803, 804, 1463, 805, 122, 2139, 2290, 1760, 2465, 123, 2424, 2058, 2230, 124, 4717, 806, 125, 126, 2370, 127, 4521, 128, 4971, 4994, 807, 4533, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 5009, 144, 145, 816, 4654, 2125, 4663, 4810, 4816, 1634, 1302, 817, 146, 818, 147, 148, 149, 4546, 150, 151, 152, 153, 154, 155, 819, 820, 156, 2149, 157, 2295, 821, 158, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 4809, 4520, 180, 826, 827, 181, 828, 4695, 2130, 4911, 182, 4582, 4873, 2189, 183, 4641, 829, 184, 185, 2244, 4647, 186, 187, 188, 189, 190, 830, 191, 192, 193, 194, 2071, 4615, 2361, 195, 4852, 4914, 4947, 196, 1391, 197, 4534, 4726, 198, 199, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2090, 2241, 217, 218, 4653, 219, 1612, 220, 221, 222, 839, 223, 4974, 4790, 4915, 2464, 4567, 4774, 4964, 2360, 840, 224, 4928, 4741, 225, 2088, 4751, 226, 227, 4890, 4519, 4883, 228, 4786, 229, 230, 231, 2448, 841, 842, 232, 233, 4782, 234, 2269, 843, 235, 844, 236, 237, 4930, 845, 846, 238, 847, 239, 848, 849, 2150, 850, 851, 852, 853, 1536, 240, 854, 241, 242, 243, 244, 245, 246, 247, 248, 4696, 855, 4634, 856, 249, 250, 251, 252, 253, 254, 255, 857, 2115, 2218, 256, 257, 258, 259, 858, 859, 260, 261, 860, 861, 862, 262, 4966, 863, 864, 263, 4749, 4970, 2296, 2318, 4535, 865, 2326, 264, 1241, 265, 266, 866, 1477, 267, 867, 268, 868, 269, 4668, 270, 869, 870, 271, 871, 872, 272, 4822, 4709, 873, 4655, 4972, 2317, 874, 1252, 273, 1591, 4975, 875, 274, 2025, 275, 1980, 276, 876, 877, 2066, 277, 278, 279, 4670, 280, 2323, 2288, 2334, 4939, 4689, 281, 878, 2416, 1892, 879, 1539, 2214, 2215, 880, 2327, 881, 882, 4601, 1295, 3601, 3877, 1930, 1420, 1678, 1460, 2306, 884, 282, 1799, 283, 284, 4988, 4871, 4553, 4772, 2260, 1626, 885, 286, 1374, 886, 2119, 2504, 2468, 287, 1620, 1290, 2425, 2482, 4564, 4671, 2282, 288, 1354, 2033, 289, 887, 290, 888, 291, 1956, 2362, 2467, 889, 2357, 4547, 4765, 292, 4794, 2391, 4324, 293, 890, 294, 295, 4138, 891, 2297, 2254, 296, 2224, 3903, 297, 892, 3829, 298, 299, 3809, 4271, 3844, 1436, 300, 1515, 1617, 301, 1961, 4649, 5010, 302, 2060, 303, 304, 305, 2452, 2147, 4574, 4538, 306, 2293, 1589, 893, 4946, 4370, 4841, 2342, 307, 308, 309, 894, 2246, 2155, 3646, 4059, 1431, 1331, 311, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 1635, 4820, 905, 331, 4706, 4484, 1756, 906, 907, 4661, 2179, 4584, 4609, 332, 4907, 4635, 4529, 4683, 4561, 333, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4319, 3906, 1533, 1755, 1888, 4248, 1557, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 2363, 908, 1935, 4711, 4728, 2460, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 337, 1184, 3450, 2577, 2567, 2532, 3415, 2934, 2984, 2568, 4147, 4943, 3002, 2986, 910, 2979, 2542, 2338, 2331, 2492, 1762, 4472, 1472, 1529, 2319, 4057, 4937, 2048, 4887, 1976, 4507, 1583, 338, 2078, 2372, 2102, 339, 911, 1711, 340, 912, 2013, 913, 2277, 914, 2403, 341, 915, 4902, 916, 342, 1594, 4440, 917, 343, 344, 4559, 918, 2439, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 371, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2345, 4471, 952, 953, 401, 2475, 403, 404, 4758, 954, 955, 2437, 4139, 1482, 3602, 4434, 3721, 1592, 1825, 1698, 1519, 1352, 4202, 4409, 1977, 1917, 405, 406, 407, 408, 956, 957, 2267, 1631, 1684, 409, 2054, 3889, 958, 410, 2117, 411, 959, 412, 960, 2443, 4712, 4619, 2285, 413, 414, 415, 961, 2352, 2417, 962, 1905, 416, 963, 417, 418, 2382, 3675, 1833, 3974, 419, 964, 420, 421, 965, 966, 967, 968, 2365, 969, 970, 422, 1897, 2056, 2309, 1272, 4754, 2207, 971, 423, 4133, 3830, 4865, 972, 424, 973, 425, 4955, 426, 974, 2109, 2380, 427, 1708, 4676, 428, 4141, 429, 430, 3749, 431, 1619, 975, 4044, 3590, 1944, 4126, 1923, 1706, 432, 976, 977, 978, 4867, 4400, 433, 434, 1606, 979, 435, 980, 2604, 2895, 981, 436, 3448, 4959, 4563, 2188, 437, 438, 2055, 1692, 982, 2388, 2243, 983, 2035, 2120, 4940, 439, 440, 441, 2474, 4900, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 2242, 453, 2286, 992, 1728, 993, 4703, 994, 995, 454, 1696, 996, 2039, 997, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 466, 2328, 467, 468, 469, 1231, 1468, 470, 471, 2496, 472, 473, 1005, 474, 2108, 4725, 1006, 476, 1273, 1007, 1744, 1488, 1659, 477, 478, 4598, 1604, 4753, 2493, 479, 1008, 1009, 1010, 1011, 1012, 1013, 1014, 481, 4313, 482, 483, 1499, 484, 1015, 4761, 2091, 2386, 2513, 1016, 4073, 1777, 2172, 485, 2205, 4592, 4545, 4720, 486, 1458, 4274, 2122, 4905, 4796, 2152, 4539, 4528, 4904, 4842, 4986, 4639, 4846, 4623, 4650, 4595, 2195, 4681, 487, 2182, 4710, 1775, 2083, 4205, 1526, 4586, 1697, 1017, 2000, 4692, 2480, 2017, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 5001, 2136, 493, 2211, 4532, 494, 2421, 495, 4090, 2378, 2210, 2247, 2478, 2047, 496, 4620, 4978, 1019, 2012, 1803, 3642, 497, 1322, 4752, 1020, 498, 1021, 2235, 2302, 2488, 4665, 4699, 4608, 4781, 499, 2049, 2404, 4685, 500, 501, 1022, 502, 1333, 503, 1023, 4686, 4804, 504, 4727, 505, 506, 4805, 2065, 1890, 1024, 507, 508, 509, 2110, 1025, 510, 1026, 3608, 1732, 511, 2397, 1864, 1816, 1531, 512, 513, 2451, 3636, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 4746, 2489, 4922, 2145, 1030, 4832, 4950, 1261, 4027, 3478, 3763, 2959, 1293, 2462, 517, 3197, 1595, 3413, 2067, 1031, 2228, 1448, 518, 4698, 4616, 2057, 2305, 2133, 2061, 4526, 4617, 1032, 1033, 2429, 2311, 1675, 1797, 519, 4578, 2271, 4693, 4604, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 1038, 536, 1039, 537, 538, 539, 1040, 3767, 1929, 1041, 1042, 3683, 2100, 4853, 2300, 2165, 540, 2105, 541, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1649, 543, 544, 545, 546, 1045, 547, 2225, 4240, 3580, 4680, 1046, 548, 549, 1047, 550, 2598, 551, 552, 553, 3819, 1259, 4062, 1048, 554, 2841, 1049, 1050, 3515, 1433, 1323, 4301, 555, 556, 1051, 557, 558, 1052, 1053, 3738, 1567, 559, 1054, 1270, 3857, 1055, 2151, 560, 2175, 2089, 4514, 2198, 2193, 1257, 1238, 3662, 561, 2186, 1057, 562, 1058, 563, 4872, 2097, 2436, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 3934, 4080, 4352, 574, 1700, 3739, 1565, 1419, 1811, 3950, 1690, 1397, 3716, 1569, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 575, 4443, 3761, 1346, 1587, 1072, 3592, 576, 1891, 4469, 1275, 577, 1950, 1963, 4129, 1073, 1296, 1360, 1551, 3733, 4111, 3993, 4108, 3949, 1287, 1713, 1556, 1736, 1995, 1435, 1424, 1705, 1861, 1197, 1254, 4120, 1545, 4417, 1469, 1292, 2185, 1785, 1236, 4293, 1827, 3839, 3983, 4395, 1957, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 2304, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2113, 590, 591, 1085, 2256, 592, 2308, 593, 594, 595, 4931, 2407, 1858, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 2321, 1086, 601, 4017, 4477, 1794, 2483, 4776, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 2320, 2069, 610, 611, 1093, 612, 1094, 3701, 4389, 613, 4897, 1603, 614, 1095, 2154, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4855, 4847, 2398, 2239, 1101, 1667, 1384, 623, 4745, 3700, 1788, 1600, 624, 2098, 625, 4550, 4721, 4960, 2473, 4792, 4675, 1102, 1423, 626, 2771, 627, 1339, 3875, 628, 629, 3070, 4565, 2118, 2015, 4818, 4860, 2557, 2795, 2906, 2103, 4869, 2029, 3299, 3118, 4932, 4838, 4901, 4908, 4996, 4779, 2377, 4657, 4646, 4875, 4627, 4515, 4876, 4965, 4605, 4576, 2028, 4985, 4799, 4850, 1103, 4757, 4747, 1614, 1947, 2237, 630, 631, 4997, 4618, 4479, 3766, 632, 1104, 1105, 4886, 1106, 2432, 4827, 633, 1107, 4530, 634, 2371, 1108, 4536, 635, 1813, 636, 2431, 4866, 4991, 4814, 4560, 4956, 637, 4555, 1109, 4633, 2213, 638, 639, 3706, 1110, 640, 1111, 641, 4581, 4893, 4831, 2481, 4644, 4734, 2190, 4881, 4837, 4888, 4648, 642, 4548, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4629, 1115, 4918, 4688, 3920, 644, 4723, 4949, 4607, 4719, 1116, 645, 646, 4611, 2353, 4603, 4762, 4819, 2221, 4636, 4992, 2307, 647, 1801, 4279, 3762, 1637, 1509, 4843, 4797, 4590, 4516, 4541, 2200, 4580, 4858, 648, 649, 2477, 4724, 4551, 4674, 1118, 1119, 2965, 2369, 2695, 3293, 650, 651, 4862, 4722, 4944, 1120, 652, 1121, 4983, 4784, 4877, 653, 654, 1122, 3264, 655, 2076, 4849, 656, 657, 2447, 658, 659, 2191, 4035, 2910, 3391, 2808, 3099, 2913, 3676, 2852, 3284, 2731, 1609, 2375, 3537, 3994, 1319, 4467, 1802, 1227, 1623, 660, 4844, 4714, 4979, 4812, 2192, 1123, 661, 2364, 662, 663, 664, 1124, 1125, 665, 2487, 4899, 2138, 1126, 666, 667, 4569, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 674, 2456, 4588, 4963, 4851, 4558, 4557, 4697, 4610, 4773, 4687, 4708, 4910, 2123, 1136, 1137, 2358, 5005, 2208, 4952, 4945, 1933, 1731, 1793, 675, 676, 3342, 1138, 1139, 677, 4840, 4597, 4870, 1140, 4990, 4954, 4916, 678, 1141, 4737, 4527, 2253, 4531, 679, 1142, 1143, 680, 4771, 1144, 4638, 681, 4705, 4525, 4898, 2325, 2415, 5004, 1145, 682, 683, 2141, 1146, 684, 685, 2445, 686, 687, 4861, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4690, 4859, 4793, 4575, 2160, 4828, 4731, 4802, 4750, 1150, 2081, 2258, 697, 1795, 1372, 1695, 1866, 1151, 4115, 1719, 2280, 4662, 698, 2176, 2217, 1920, 699, 700, 701, 4999, 1544, 4739, 2512, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 2340, 4182, 706, 4652, 2442, 2484, 707, 708, 709, 4667, 1153, 2412, 4732, 4476, 2053, 710, 1375, 711, 712, 713, 4738, 4811, 4570, 1245, 714, 2045, 4707, 715, 2169, 4713, 1841, 4234, 4924, 1154, 1314, 4096, 2023, 717, 4895, 2414, 718, 1155, 719, 720, 2063, 1311, 4921, 721, 4833, 4903, 4885, 4666, 2249, 4217, 1156, 723, 1157, 1627, 2116, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 2248, 730, 731, 1846, 2507, 2368, 1162, 2019, 2333, 3815, 2005, 2266, 1964, 2347, 732, 2177, 2059, 4958, 4927, 1822, 4815, 1262, 2094, 4480, 1537, 1291, 2419, 1318, 4628, 4294, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4835, 2259, 2275, 1166, 4968, 2423, 737, 1167, 4989, 4612, 2127, 1745, 1973, 4767, 1168, 2273, 2310, 3807, 4307, 1169, 2303, 1454, or 1781.

In some aspects, (a) X³ of SEQ ID NO: 5014 is not E, (b) X⁶ of SEQ ID NO: 5014 is not E, or (c) X³ of SEQ ID NO: 5014 is not E and X⁶ of SEQ ID NO: 5014 is not E. In some aspects, the 581 to 589 region of the VP capsid polypeptide has a sequence of any one of SEQ ID NOs: 2498, 4884, 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 4889, 21, 3192, 4942, 22, 23, 1638, 748, 24, 25, 749, 26, 27, 1703, 1836, 28, 2038, 2301, 3660, 29, 750, 30, 31, 32, 2095, 1640, 33, 34, 35, 36, 37, 4742, 2450, 38, 4925, 39, 751, 752, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2080, 2072, 2034, 50, 1308, 1230, 3960, 1378, 2994, 3080, 2589, 3681, 51, 4694, 2257, 52, 760, 53, 2389, 4934, 4951, 761, 4591, 762, 4659, 54, 2146, 3925, 55, 3654, 2087, 2346, 4766, 4682, 4984, 4744, 56, 763, 764, 4626, 4702, 57, 58, 59, 60, 61, 4086, 1740, 1660, 765, 766, 62, 1966, 2032, 2499, 2332, 767, 63, 2096, 2181, 4913, 64, 768, 769, 770, 2384, 2402, 771, 65, 66, 2111, 67, 772, 2458, 2197, 68, 3816, 69, 2599, 2706, 2497, 70, 2234, 2183, 2399, 71, 4769, 2366, 72, 73, 74, 3865, 1549, 1361, 2453, 3882, 773, 3713, 1942, 2140, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2132, 2379, 81, 82, 4065, 83, 84, 2030, 1294, 2413, 2064, 1484, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 93, 2356, 784, 785, 786, 94, 95, 787, 788, 1831, 1867, 96, 4458, 97, 1970, 4656, 2173, 1936, 2349, 1590, 4585, 98, 99, 100, 789, 790, 1298, 101, 1688, 791, 792, 2007, 4556, 2070, 102, 793, 4632, 103, 794, 795, 104, 105, 106, 2229, 796, 4980, 4691, 107, 2440, 1849, 108, 2014, 797, 4736, 2509, 2018, 2227, 4787, 109, 1579, 110, 1906, 111, 2428, 798, 2075, 112, 2411, 113, 2359, 799, 4848, 800, 2461, 1444, 2313, 2350, 4579, 4821, 801, 114, 2329, 4834, 115, 1344, 116, 802, 117, 118, 119, 4929, 4677, 120, 2158, 121, 803, 804, 1463, 805, 122, 2139, 2290, 1760, 2465, 123, 2424, 2058, 2230, 124, 4717, 806, 125, 126, 2370, 127, 4521, 128, 4971, 4994, 807, 2085, 4533, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 4544, 5009, 144, 145, 816, 4654, 4816, 1634, 1302, 817, 146, 818, 147, 148, 149, 4546, 150, 151, 152, 153, 154, 155, 819, 820, 156, 2149, 157, 2295, 821, 158, 159, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 2170, 4809, 4520, 180, 826, 827, 181, 828, 4695, 4911, 182, 4582, 4873, 4596, 2020, 183, 4641, 829, 184, 185, 2244, 4647, 186, 187, 188, 189, 190, 2114, 830, 191, 192, 193, 194, 2071, 4615, 2361, 195, 4852, 2187, 4914, 4947, 196, 1391, 197, 4534, 4726, 4788, 198, 199, 4920, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2241, 217, 218, 4653, 219, 1612, 220, 221, 222, 839, 223, 4953, 2360, 840, 224, 4928, 4741, 225, 2088, 4751, 5007, 4542, 226, 227, 4890, 4519, 4883, 228, 4786, 229, 230, 231, 2393, 2448, 841, 842, 232, 233, 4782, 234, 4962, 2269, 843, 235, 844, 236, 237, 4930, 845, 846, 238, 847, 4704, 239, 848, 849, 2150, 850, 851, 852, 853, 1536, 240, 854, 241, 242, 243, 244, 245, 246, 247, 248, 4923, 2245, 4696, 855, 4634, 856, 249, 250, 251, 252, 253, 254, 255, 857, 2115, 4880, 2218, 256, 257, 258, 259, 858, 859, 260, 261, 860, 861, 862, 262, 4966, 863, 864, 263, 4749, 4970, 2296, 2318, 865, 2326, 264, 1241, 265, 266, 866, 1477, 267, 867, 4977, 268, 868, 269, 4668, 5002, 270, 869, 870, 271, 871, 872, 272, 4822, 4709, 873, 4655, 4972, 2392, 874, 1252, 273, 1591, 4975, 875, 274, 2025, 275, 1980, 276, 876, 877, 2066, 277, 278, 279, 4670, 280, 2323, 2288, 2334, 4939, 4689, 281, 878, 1892, 879, 1539, 2214, 2215, 880, 2327, 881, 882, 4601, 883, 1295, 3601, 3877, 1930, 1420, 1678, 1460, 2306, 884, 282, 1799, 283, 284, 4988, 4553, 4772, 4817, 2260, 285, 1626, 2082, 885, 286, 1374, 886, 2119, 2468, 287, 2408, 1620, 1290, 2425, 2482, 4564, 4671, 2282, 288, 2354, 1354, 2033, 289, 887, 290, 888, 291, 1956, 2362, 2467, 889, 2357, 4547, 4765, 292, 4794, 2391, 4324, 293, 890, 294, 295, 4138, 891, 2297, 2254, 296, 2224, 3903, 297, 892, 3829, 298, 299, 3809, 4271, 3844, 1436, 300, 1515, 1617, 4701, 2174, 301, 1961, 4649, 5010, 302, 303, 304, 305, 2452, 2147, 306, 2293, 1589, 893, 4370, 4841, 2342, 307, 308, 309, 894, 2246, 2155, 3646, 4059, 1431, 1331, 310, 311, 4836, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 904, 1635, 4820, 905, 2506, 331, 4706, 2092, 4484, 1756, 906, 907, 4661, 2179, 4584, 4609, 332, 4907, 4635, 4529, 4683, 4523, 4561, 333, 2337, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4783, 4319, 3906, 1533, 1755, 1888, 4248, 1557, 2153, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 2444, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 2363, 908, 1935, 4711, 4728, 2460, 4517, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 910, 2979, 2542, 2338, 2492, 1762, 4472, 1472, 1529, 2319, 2131, 4057, 4937, 2048, 1976, 4507, 1583, 338, 2078, 2372, 339, 911, 1711, 340, 912, 2013, 913, 2277, 914, 2403, 341, 915, 4672, 4902, 916, 342, 1594, 4440, 917, 343, 344, 4559, 918, 2233, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 2471, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 371, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2343, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 2383, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2129, 2345, 4471, 952, 953, 402, 2475, 403, 404, 4758, 955, 2437, 4139, 1482, 3602, 4434, 3721, 1592, 1825, 1698, 1519, 1352, 4202, 4409, 1977, 1917, 405, 406, 407, 408, 956, 2272, 957, 2267, 1631, 1684, 409, 2054, 3889, 958, 410, 2117, 411, 959, 412, 960, 2443, 4712, 4619, 2285, 414, 415, 961, 2352, 2417, 962, 1905, 416, 963, 417, 418, 3675, 1833, 3974, 419, 964, 420, 421, 965, 966, 967, 968, 2365, 969, 970, 422, 1897, 2056, 2309, 4733, 1272, 4754, 2207, 971, 423, 4133, 3830, 4865, 972, 424, 973, 425, 2068, 4955, 426, 974, 427, 1708, 4676, 428, 4141, 430, 3749, 431, 1619, 975, 4044, 3590, 1944, 4126, 1923, 1706, 432, 976, 977, 978, 4867, 4400, 433, 434, 1606, 2143, 979, 435, 980, 2604, 2895, 981, 436, 3448, 4959, 4563, 2188, 437, 438, 1692, 982, 2388, 2433, 2243, 983, 2035, 2120, 4940, 439, 440, 441, 2474, 442, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 2242, 453, 2286, 992, 1728, 993, 4703, 994, 995, 454, 1696, 996, 2039, 455, 997, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 467, 468, 469, 1231, 1468, 470, 471, 2496, 472, 473, 1005, 474, 475, 2108, 4725, 1006, 476, 1273, 1007, 1744, 1488, 1659, 477, 478, 4598, 1604, 4753, 2493, 479, 1008, 1009, 480, 1010, 1011, 1012, 1013, 1014, 481, 4313, 482, 483, 1499, 484, 1015, 4761, 2091, 2513, 1016, 4073, 1777, 2172, 485, 2205, 4592, 4545, 4720, 486, 1458, 4274, 2122, 4905, 5003, 4599, 4796, 2152, 4539, 4528, 4904, 4842, 4986, 4639, 4846, 4623, 4650, 4595, 2195, 4681, 2142, 487, 2182, 4710, 1775, 2083, 4205, 1526, 4586, 4602, 4518, 1697, 1017, 2000, 4692, 4795, 2480, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 2202, 5001, 2136, 493, 2211, 4532, 494, 2421, 495, 4090, 2378, 2104, 2210, 2247, 2478, 2047, 496, 4620, 4978, 4614, 1019, 2012, 1803, 3642, 497, 1322, 1020, 498, 1021, 2235, 2302, 2488, 4665, 4540, 5006, 499, 2049, 2404, 4685, 500, 501, 1022, 502, 1333, 503, 1023, 4686, 4804, 504, 4727, 505, 506, 4938, 4805, 2065, 1890, 4891, 1024, 507, 508, 509, 1025, 510, 1026, 3608, 1732, 511, 2397, 1864, 1816, 1531, 512, 513, 2451, 3636, 5012, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 2093, 4746, 2489, 4922, 2145, 1030, 4832, 4950, 1261, 4027, 3478, 3763, 2959, 1293, 2462, 517, 3197, 1595, 3413, 2238, 1031, 2228, 1448, 518, 2057, 2305, 2133, 2061, 4526, 4617, 1033, 2429, 2311, 1675, 4785, 1797, 519, 4578, 2271, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 535, 1038, 536, 1039, 537, 538, 539, 1040, 3767, 1929, 1041, 1042, 3683, 2100, 4853, 2300, 2165, 540, 541, 1043, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1649, 543, 544, 545, 546, 1045, 547, 2084, 4240, 3580, 4680, 1046, 548, 2062, 1047, 550, 2598, 551, 552, 553, 3819, 1259, 4062, 1048, 554, 2841, 1049, 1050, 3515, 2219, 1433, 1323, 4301, 555, 556, 1051, 557, 558, 1052, 1053, 3738, 1567, 559, 1054, 1270, 3857, 1055, 2151, 560, 2175, 2089, 1056, 2198, 2193, 1257, 1238, 3662, 561, 2186, 1057, 4637, 562, 1058, 563, 4872, 2097, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 2304, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2255, 2113, 590, 591, 1085, 2256, 592, 2308, 593, 594, 2274, 595, 4931, 2407, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 2321, 1086, 601, 4017, 4477, 1794, 2483, 4776, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 610, 611, 1093, 612, 1094, 3701, 4389, 613, 4897, 1603, 614, 1095, 2154, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4801, 2398, 2239, 1101, 1667, 1384, 623, 4745, 3700, 1788, 1600, 624, 2098, 625, 4550, 4721, 4960, 4878, 2473, 4792, 4675, 1102, 1423, 626, 2771, 627, 1339, 3875, 628, 629, 3070, 4565, 2118, 4818, 4860, 2557, 2795, 2906, 2103, 4869, 2029, 3299, 3118, 4932, 4838, 4901, 2377, 4657, 4646, 4875, 4627, 4515, 4876, 4965, 4605, 4576, 2028, 4985, 4799, 4850, 1103, 4747, 1614, 1947, 2237, 630, 631, 4618, 4479, 3766, 632, 1104, 1105, 4886, 1106, 2432, 4827, 633, 1107, 4530, 634, 2371, 4651, 1108, 4536, 635, 1813, 636, 2431, 4866, 4991, 4814, 4560, 4956, 637, 4555, 1109, 4941, 4577, 2213, 638, 639, 3706, 1110, 640, 1111, 641, 4581, 4893, 4831, 2481, 4644, 4734, 2190, 4524, 4881, 4837, 4888, 4648, 642, 4548, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4760, 1115, 4918, 4688, 3920, 644, 4723, 4949, 4748, 4607, 4719, 1116, 645, 646, 4611, 2353, 4645, 4998, 2307, 647, 1801, 4279, 3762, 1637, 1509, 4843, 4797, 4590, 4516, 4995, 4541, 2200, 4580, 4858, 648, 649, 2502, 1117, 4780, 4800, 4551, 4674, 1118, 1119, 2965, 2369, 2695, 3293, 650, 651, 4730, 4862, 4722, 4944, 1120, 652, 1121, 4983, 4784, 4877, 4643, 653, 654, 4909, 1122, 3264, 655, 2076, 4849, 656, 657, 2447, 658, 659, 2191, 4035, 2910, 3391, 2808, 3099, 2913, 3676, 2852, 3284, 2731, 1609, 2375, 2201, 3537, 3994, 1319, 4467, 1802, 1227, 1623, 660, 4844, 2086, 4770, 2192, 1123, 661, 2364, 662, 663, 664, 1124, 1125, 665, 2487, 4899, 2138, 1126, 666, 667, 4569, 4554, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 674, 2456, 4606, 4910, 2123, 1136, 1137, 2358, 5005, 2208, 4952, 4945, 1933, 1731, 1793, 675, 676, 3342, 1138, 1139, 677, 4840, 4597, 4870, 1140, 4990, 4954, 4916, 678, 1141, 4737, 4527, 2253, 4531, 679, 1142, 1143, 680, 4771, 1144, 4638, 681, 4705, 4525, 4522, 4898, 2325, 2415, 5004, 1145, 682, 683, 1146, 684, 685, 2445, 686, 687, 4861, 2157, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4793, 4575, 2160, 4828, 4731, 4718, 4802, 4750, 1150, 2081, 2258, 697, 1795, 1372, 1695, 1866, 1151, 4115, 1719, 2280, 4662, 698, 2176, 2194, 2217, 1920, 699, 700, 701, 4999, 1544, 4739, 2512, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 4824, 705, 2340, 4182, 706, 4652, 2442, 2484, 707, 708, 709, 4667, 1153, 2412, 4476, 2053, 710, 1375, 711, 712, 713, 4829, 4738, 4811, 4570, 4961, 1245, 714, 2045, 4707, 715, 2169, 4713, 4879, 1841, 4234, 4924, 1154, 1314, 4096, 2023, 716, 717, 4895, 2414, 718, 1155, 719, 720, 2063, 1311, 4921, 721, 4642, 722, 4885, 4666, 2249, 4217, 1156, 723, 1157, 1627, 2116, 4566, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 730, 731, 2024, 1846, 2507, 2368, 2019, 2333, 3815, 2005, 2266, 1964, 2347, 2385, 732, 2177, 2059, 4958, 1822, 4815, 1262, 2094, 4480, 1537, 1291, 2419, 1318, 2283, 4976, 4294, 2486, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4679, 4835, 2259, 4839, 4571, 2275, 1166, 4968, 2250, 4912, 2423, 737, 1167, 4989, 4684, 4669, 4552, 4612, 2127, 1745, 1973, 4767, 1168, 2273, 2310, 3807, 4307, 1169, 2303, 1454, or 1781. In some aspects, the 581 to 589 region of the VP capsid polypeptide has a sequence of any one of SEQ ID NOs: 2498, 4884, 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 2469, 4889, 21, 3192, 4942, 22, 23, 1638, 748, 24, 25, 749, 26, 27, 1703, 1836, 28, 2038, 2301, 3660, 29, 750, 30, 31, 32, 2095, 1640, 33, 35, 36, 37, 4742, 2450, 38, 4925, 39, 751, 752, 2144, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2080, 50, 1308, 1230, 3960, 1378, 2994, 3080, 2589, 3681, 51, 4694, 2257, 2167, 52, 760, 53, 2389, 4934, 4951, 4755, 761, 762, 4659, 54, 2146, 3925, 55, 3654, 2087, 2346, 4766, 4682, 4984, 4744, 56, 763, 764, 4626, 4702, 58, 59, 60, 61, 4086, 1740, 1660, 765, 766, 62, 1966, 2032, 2499, 2332, 767, 63, 2096, 2181, 4913, 2438, 2041, 64, 768, 769, 770, 2384, 2402, 771, 65, 66, 2111, 67, 772, 2458, 2197, 68, 3816, 69, 2599, 2706, 2497, 70, 2234, 2183, 2399, 71, 4769, 2366, 72, 73, 74, 3865, 1549, 1361, 2453, 3882, 773, 3713, 1942, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2132, 2379, 82, 4065, 83, 84, 2030, 1294, 2413, 2064, 1484, 2299, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 93, 2356, 784, 785, 786, 94, 95, 787, 788, 1831, 1867, 96, 4458, 97, 1970, 4656, 2173, 1936, 2349, 1590, 4585, 98, 99, 100, 789, 790, 1298, 101, 1688, 791, 792, 2007, 2070, 102, 793, 4632, 103, 794, 795, 104, 105, 106, 2229, 796, 4980, 4691, 107, 2440, 1849, 108, 2014, 797, 4736, 2509, 2018, 2227, 4787, 109, 1579, 110, 1906, 111, 2428, 798, 112, 2411, 113, 2359, 799, 4848, 800, 2461, 1444, 2313, 2350, 4579, 4821, 801, 114, 2329, 4864, 4834, 115, 1344, 116, 802, 117, 118, 119, 4929, 4677, 120, 4791, 2158, 121, 803, 804, 1463, 805, 122, 2139, 2290, 1760, 2465, 2424, 2058, 2230, 124, 4717, 806, 125, 126, 2370, 127, 4521, 128, 4971, 4994, 807, 2085, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 4544, 144, 145, 816, 4654, 2125, 4663, 4810, 4816, 1634, 1302, 817, 146, 818, 147, 148, 149, 4546, 150, 151, 152, 153, 154, 155, 819, 820, 156, 2149, 157, 2295, 821, 158, 159, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 2170, 4809, 4520, 180, 826, 827, 181, 828, 4695, 2130, 4911, 182, 4582, 4873, 4596, 2020, 2189, 183, 4641, 829, 184, 185, 2244, 4647, 186, 187, 188, 189, 190, 2114, 830, 191, 192, 193, 194, 2071, 4615, 2361, 195, 4852, 2187, 4914, 4947, 196, 1391, 197, 4534, 4726, 4788, 198, 199, 4920, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2090, 2241, 217, 218, 4653, 219, 1612, 220, 221, 222, 839, 223, 4953, 4974, 4790, 4915, 2464, 4567, 4774, 4964, 2360, 840, 224, 4928, 4741, 225, 2088, 4751, 5007, 4542, 226, 227, 4890, 4519, 4883, 228, 4786, 229, 230, 231, 2393, 2448, 841, 842, 232, 233, 4782, 234, 4962, 2269, 843, 235, 844, 236, 237, 4930, 845, 846, 238, 847, 4704, 239, 848, 849, 2150, 850, 851, 852, 853, 1536, 240, 854, 241, 242, 243, 244, 245, 246, 247, 248, 4923, 2245, 4696, 855, 4634, 856, 249, 250, 251, 252, 253, 254, 255, 857, 2115, 4880, 256, 257, 258, 259, 858, 859, 260, 261, 860, 861, 862, 262, 4966, 863, 864, 263, 4749, 4970, 2296, 2318, 4535, 865, 2326, 264, 1241, 265, 266, 866, 1477, 267, 867, 4977, 268, 868, 269, 4668, 5002, 270, 869, 870, 271, 871, 872, 272, 4822, 4709, 873, 4655, 4972, 2392, 2317, 874, 1252, 273, 1591, 4975, 875, 274, 2025, 275, 1980, 276, 876, 877, 2066, 277, 278, 279, 4670, 280, 2323, 2288, 2334, 4689, 281, 878, 2416, 1892, 879, 1539, 2214, 2215, 880, 2327, 881, 882, 4601, 883, 1295, 3601, 3877, 1930, 1420, 1678, 1460, 2306, 884, 282, 1799, 283, 284, 4988, 4871, 4553, 4772, 4817, 2260, 285, 1626, 2082, 885, 286, 1374, 886, 2119, 2504, 2468, 287, 1620, 1290, 2425, 2482, 4671, 2282, 288, 2354, 1354, 2033, 289, 887, 290, 888, 291, 1956, 2467, 889, 2357, 4547, 4765, 292, 4794, 2391, 4324, 890, 294, 295, 4138, 891, 2297, 296, 2224, 3903, 297, 892, 3829, 298, 299, 3809, 4271, 3844, 1436, 300, 1515, 1617, 4701, 2174, 301, 1961, 4649, 5010, 302, 2060, 303, 304, 305, 2452, 2147, 4574, 4538, 306, 2293, 1589, 893, 4946, 4370, 4841, 2342, 307, 308, 309, 894, 2246, 2155, 3646, 4059, 1431, 1331, 310, 311, 4836, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 904, 1635, 4820, 905, 2506, 331, 4706, 2092, 4484, 1756, 906, 907, 4661, 2179, 4584, 332, 4907, 4635, 4529, 4683, 4523, 4561, 333, 2337, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4783, 4319, 3906, 1533, 1755, 1888, 4248, 1557, 2153, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 2444, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 908, 1935, 4711, 4728, 2460, 4517, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 4943, 3002, 2986, 910, 2979, 2542, 2331, 2492, 1762, 4472, 1472, 1529, 2319, 2131, 4057, 2048, 4887, 1976, 4507, 1583, 338, 2078, 2372, 2102, 339, 911, 1711, 340, 912, 2013, 913, 2277, 914, 341, 915, 4672, 4902, 916, 342, 1594, 4440, 917, 343, 344, 4559, 918, 2233, 2439, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 2471, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 375, 2343, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 2383, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2129, 2345, 4471, 952, 953, 401, 402, 2475, 403, 404, 4758, 955, 2437, 4139, 1482, 3602, 4434, 3721, 1592, 1825, 1698, 1519, 1352, 4202, 4409, 1977, 1917, 405, 406, 407, 408, 956, 2272, 957, 2267, 1631, 1684, 409, 2054, 3889, 958, 410, 2117, 411, 959, 412, 960, 2443, 4712, 4619, 2285, 413, 414, 415, 961, 2352, 2417, 962, 1905, 416, 963, 417, 418, 2382, 3675, 1833, 3974, 419, 964, 421, 965, 966, 967, 4919, 968, 2365, 969, 970, 422, 1897, 2056, 2309, 4733, 1272, 4754, 2207, 971, 423, 4133, 3830, 4865, 972, 424, 973, 425, 2068, 4955, 426, 974, 2109, 2380, 427, 1708, 4676, 428, 4141, 429, 430, 3749, 431, 1619, 975, 4044, 3590, 1944, 4126, 1923, 1706, 432, 976, 977, 978, 4867, 4400, 433, 434, 1606, 2143, 979, 435, 980, 2604, 2895, 981, 436, 3448, 4959, 2188, 437, 438, 2055, 1692, 982, 2388, 2433, 2243, 983, 2035, 2120, 4940, 439, 440, 2474, 442, 4900, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 453, 992, 1728, 993, 4703, 994, 995, 454, 1696, 996, 2039, 455, 997, 4874, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 466, 2328, 467, 468, 469, 1231, 1468, 470, 471, 2496, 472, 473, 1005, 474, 475, 2108, 4725, 476, 1273, 1007, 1744, 1488, 1659, 477, 478, 4598, 1604, 4753, 2493, 479, 1008, 1009, 480, 1011, 1012, 1013, 1014, 481, 4313, 482, 483, 1499, 484, 1015, 4761, 2091, 2386, 2513, 1016, 4073, 1777, 2172, 485, 2205, 4592, 4545, 486, 1458, 4274, 2122, 4905, 5003, 4599, 4796, 2152, 4539, 4528, 4904, 4842, 4986, 4846, 4650, 4595, 2195, 4681, 2142, 487, 4710, 1775, 4205, 1526, 4586, 4602, 4518, 1697, 1017, 2000, 4692, 4795, 2480, 2017, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 2202, 5001, 2136, 493, 2211, 4532, 494, 2421, 495, 4090, 2378, 2104, 2210, 2247, 2478, 2047, 496, 4620, 4978, 4614, 1019, 2012, 1803, 3642, 497, 1322, 4752, 1020, 498, 1021, 2235, 2302, 2488, 4665, 4540, 5006, 4699, 4608, 4781, 499, 2049, 4685, 500, 501, 1022, 502, 1333, 503, 1023, 4686, 4804, 504, 4727, 505, 506, 4938, 4805, 2065, 1890, 4891, 1024, 507, 508, 509, 2110, 1025, 510, 1026, 3608, 1732, 511, 2397, 1864, 1816, 1531, 512, 513, 2451, 3636, 5012, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 2093, 4746, 2489, 4922, 2145, 1030, 4832, 4950, 1261, 4027, 3478, 3763, 2959, 1293, 2462, 517, 3197, 1595, 3413, 2238, 2067, 1031, 2228, 518, 4698, 4616, 2057, 2305, 2133, 2061, 4526, 4617, 1032, 1033, 2429, 2311, 1675, 4785, 1797, 519, 4578, 2271, 4693, 4604, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 535, 1038, 536, 1039, 537, 538, 539, 1040, 3767, 1929, 1042, 3683, 2100, 4853, 2300, 2165, 540, 2105, 541, 1043, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 1044, 3720, 4351, 4414, 1673, 3453, 3254, 2501, 1649, 543, 544, 545, 546, 1045, 547, 4240, 3580, 4680, 1046, 548, 2062, 549, 1047, 550, 2598, 551, 552, 553, 3819, 1259, 4062, 1048, 554, 2841, 1049, 1050, 3515, 2219, 1433, 1323, 4301, 555, 556, 1051, 557, 558, 1052, 1053, 3738, 1567, 559, 1054, 1270, 3857, 1055, 2151, 560, 2175, 2089, 1056, 4514, 2198, 2193, 1257, 1238, 3662, 561, 2186, 1057, 4637, 562, 1058, 563, 4872, 2097, 2436, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2185, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2255, 2113, 590, 591, 1085, 592, 2308, 593, 594, 2274, 595, 4931, 2407, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 1086, 601, 4017, 4477, 1794, 2483, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 2320, 2069, 610, 611, 1093, 612, 1094, 3701, 4389, 613, 4897, 1603, 614, 1095, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4801, 4855, 4847, 2398, 2239, 1101, 1667, 1384, 623, 4745, 3700, 1788, 1600, 624, 2098, 625, 4550, 4721, 4960, 4878, 2473, 4792, 4675, 1102, 1423, 626, 2771, 627, 1339, 3875, 628, 629, 3070, 4565, 2118, 2015, 4818, 2557, 2795, 2906, 2103, 4869, 2029, 3299, 3118, 4932, 4838, 4901, 4908, 4996, 4779, 4657, 4646, 4875, 4627, 4515, 4876, 4965, 4605, 4576, 2028, 4985, 4850, 1103, 4757, 4747, 1614, 1947, 2237, 630, 631, 4997, 4618, 4479, 3766, 632, 1104, 1105, 4886, 1106, 2432, 633, 1107, 4530, 634, 2371, 4651, 1108, 4536, 635, 1813, 636, 2431, 4866, 4991, 4814, 4560, 4956, 637, 4555, 1109, 4941, 4577, 4633, 2213, 638, 639, 3706, 1110, 640, 1111, 641, 4581, 4893, 4831, 2481, 4644, 4734, 2190, 4524, 4881, 4837, 4888, 4648, 642, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4760, 4629, 1115, 4918, 4688, 3920, 644, 4723, 4949, 4748, 4607, 4719, 1116, 645, 646, 4611, 2353, 4645, 4603, 4762, 4819, 2221, 4636, 4992, 4998, 2307, 647, 1801, 4279, 3762, 1637, 1509, 4843, 4797, 4590, 4995, 4541, 2200, 4580, 4858, 648, 649, 2502, 1117, 4780, 4800, 2477, 4724, 4551, 4674, 1118, 1119, 2965, 2369, 2695, 3293, 650, 651, 4730, 4862, 4722, 4944, 1120, 652, 1121, 4983, 4784, 4877, 4643, 653, 654, 4909, 1122, 3264, 655, 2076, 4849, 656, 657, 2447, 658, 659, 4035, 2910, 3391, 2808, 3099, 2913, 3676, 2852, 3284, 2731, 1609, 2375, 2201, 3537, 3994, 1319, 4467, 1802, 1227, 1623, 660, 4844, 2086, 4770, 4714, 4979, 4812, 2192, 1123, 661, 2364, 662, 663, 664, 1124, 1125, 665, 2487, 4899, 2138, 1126, 666, 667, 4569, 4554, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 2456, 4606, 4588, 4963, 4851, 4558, 4557, 4697, 4610, 4773, 4687, 4708, 4910, 2123, 1136, 1137, 2358, 5005, 2208, 4952, 4945, 1933, 1731, 1793, 675, 676, 3342, 1138, 677, 4597, 4870, 1140, 4990, 4954, 4916, 678, 1141, 4737, 4527, 2253, 4531, 679, 1142, 1143, 680, 4771, 1144, 4638, 681, 4705, 4525, 4522, 4898, 2325, 2415, 5004, 1145, 682, 683, 2141, 1146, 684, 685, 2445, 686, 687, 4861, 2157, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4690, 4859, 4793, 4575, 2160, 4828, 4731, 4718, 4802, 4750, 1150, 2081, 2258, 697, 1795, 1372, 1695, 1866, 1151, 4115, 1719, 2280, 4662, 698, 2176, 2194, 2217, 1920, 699, 700, 701, 4999, 1544, 4739, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 4824, 705, 2340, 4182, 706, 2442, 2484, 707, 708, 709, 4667, 1153, 2412, 4732, 4476, 2053, 710, 1375, 711, 712, 713, 4829, 4738, 4811, 4570, 4961, 1245, 714, 2045, 4707, 715, 2169, 4713, 1841, 4234, 4924, 1154, 1314, 4096, 2023, 716, 717, 4895, 718, 1155, 719, 720, 2063, 1311, 721, 4642, 722, 4833, 4903, 4885, 4666, 2249, 4217, 1156, 723, 1157, 1627, 2116, 4566, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 2248, 730, 731, 2024, 1846, 2507, 2368, 1162, 2019, 2333, 3815, 2005, 2266, 1964, 2347, 2385, 732, 2177, 2059, 4958, 4927, 1822, 4815, 1262, 2094, 4480, 1537, 1291, 2419, 1318, 2283, 4976, 4628, 4294, 2486, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4679, 4835, 2259, 4839, 4571, 1166, 4968, 2250, 4912, 2423, 737, 1167, 4989, 4684, 4669, 4552, 4612, 2127, 1745, 1973, 4767, 1168, 2273, 2310, 3807, 4307, 1169, 2303, 1454, or 1781. In some aspects, the 581 to 589 region of the VP capsid polypeptide has a sequence of any one of SEQ ID NOs: 2498, 4884, 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 4889, 21, 3192, 4942, 22, 23, 1638, 748, 24, 25, 749, 26, 27, 1703, 1836, 28, 2038, 2301, 3660, 29, 750, 30, 31, 32, 2095, 1640, 33, 35, 36, 37, 4742, 2450, 38, 4925, 39, 751, 752, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2080, 50, 1308, 1230, 3960, 1378, 2994, 3080, 2589, 3681, 51, 4694, 2257, 52, 760, 53, 2389, 4934, 4951, 761, 762, 4659, 54, 2146, 3925, 55, 3654, 2087, 2346, 4766, 4682, 4984, 4744, 56, 763, 764, 4626, 4702, 58, 59, 60, 61, 4086, 1740, 1660, 765, 766, 62, 1966, 2032, 2499, 2332, 767, 63, 2096, 2181, 4913, 64, 768, 769, 770, 2384, 2402, 771, 65, 66, 2111, 67, 772, 2458, 2197, 68, 3816, 69, 2599, 2706, 2497, 70, 2234, 2183, 2399, 71, 4769, 2366, 72, 73, 74, 3865, 1549, 1361, 2453, 3882, 773, 3713, 1942, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2132, 2379, 82, 4065, 83, 84, 2030, 1294, 2413, 2064, 1484, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 93, 2356, 784, 785, 786, 94, 95, 787, 788, 1831, 1867, 96, 4458, 97, 1970, 4656, 2173, 1936, 2349, 1590, 4585, 98, 99, 100, 789, 790, 1298, 101, 1688, 791, 792, 2007, 2070, 102, 793, 4632, 103, 794, 795, 104, 105, 106, 2229, 796, 4980, 4691, 107, 2440, 1849, 108, 2014, 797, 4736, 2509, 2018, 2227, 4787, 109, 1579, 110, 1906, 111, 2428, 798, 112, 2411, 113, 2359, 799, 4848, 800, 2461, 1444, 2313, 2350, 4579, 4821, 801, 114, 2329, 4834, 115, 1344, 116, 802, 117, 118, 119, 4929, 4677, 120, 2158, 121, 803, 804, 1463, 805, 122, 2139, 2290, 1760, 2465, 2424, 2058, 2230, 124, 4717, 806, 125, 126, 2370, 127, 4521, 128, 4971, 4994, 807, 2085, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 4544, 144, 145, 816, 4654, 4816, 1634, 1302, 817, 146, 818, 147, 148, 149, 4546, 150, 151, 152, 153, 154, 155, 819, 820, 156, 2149, 157, 2295, 821, 158, 159, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 2170, 4809, 4520, 180, 826, 827, 181, 828, 4695, 4911, 182, 4582, 4873, 4596, 2020, 183, 4641, 829, 184, 185, 2244, 4647, 186, 187, 188, 189, 190, 2114, 830, 191, 192, 193, 194, 2071, 4615, 2361, 195, 4852, 2187, 4914, 4947, 196, 1391, 197, 4534, 4726, 4788, 198, 199, 4920, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2241, 217, 218, 4653, 219, 1612, 220, 221, 222, 839, 223, 4953, 2360, 840, 224, 4928, 4741, 225, 2088, 4751, 5007, 4542, 226, 227, 4890, 4519, 4883, 228, 4786, 229, 230, 231, 2393, 2448, 841, 842, 232, 233, 4782, 234, 4962, 2269, 843, 235, 844, 236, 237, 4930, 845, 846, 238, 847, 4704, 239, 848, 849, 2150, 850, 851, 852, 853, 1536, 240, 854, 241, 242, 243, 244, 245, 246, 247, 248, 4923, 2245, 4696, 855, 4634, 856, 249, 250, 251, 252, 253, 254, 255, 857, 2115, 4880, 256, 257, 258, 259, 858, 859, 260, 261, 860, 861, 862, 262, 4966, 863, 864, 263, 4749, 4970, 2296, 2318, 865, 2326, 264, 1241, 265, 266, 866, 1477, 267, 867, 4977, 268, 868, 269, 4668, 5002, 270, 869, 870, 271, 871, 872, 272, 4822, 4709, 873, 4655, 4972, 2392, 874, 1252, 273, 1591, 4975, 875, 274, 2025, 275, 1980, 276, 876, 877, 2066, 277, 278, 279, 4670, 280, 2323, 2288, 2334, 4689, 281, 878, 1892, 879, 1539, 2214, 2215, 880, 2327, 881, 882, 4601, 883, 1295, 3601, 3877, 1930, 1420, 1678, 1460, 2306, 884, 282, 1799, 283, 284, 4988, 4553, 4772, 4817, 2260, 285, 1626, 2082, 885, 286, 1374, 886, 2119, 2468, 287, 1620, 1290, 2425, 2482, 4671, 2282, 288, 2354, 1354, 2033, 289, 887, 290, 888, 291, 1956, 2467, 889, 2357, 4547, 4765, 292, 4794, 2391, 4324, 890, 294, 295, 4138, 891, 2297, 296, 2224, 3903, 297, 892, 3829, 298, 299, 3809, 4271, 3844, 1436, 300, 1515, 1617, 4701, 2174, 301, 1961, 4649, 5010, 302, 303, 304, 305, 2452, 2147, 306, 2293, 1589, 893, 4370, 4841, 2342, 307, 308, 309, 894, 2246, 2155, 3646, 4059, 1431, 1331, 310, 311, 4836, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 904, 1635, 4820, 905, 2506, 331, 4706, 2092, 4484, 1756, 906, 907, 4661, 2179, 4584, 332, 4907, 4635, 4529, 4683, 4523, 4561, 333, 2337, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4783, 4319, 3906, 1533, 1755, 1888, 4248, 1557, 2153, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 2444, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 908, 1935, 4711, 4728, 2460, 4517, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 910, 2979, 2542, 2492, 1762, 4472, 1472, 1529, 2319, 2131, 4057, 2048, 1976, 4507, 1583, 338, 2078, 2372, 339, 911, 1711, 340, 912, 2013, 913, 2277, 914, 341, 915, 4672, 4902, 916, 342, 1594, 4440, 917, 343, 344, 4559, 918, 2233, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 2471, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 375, 2343, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 2383, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2129, 2345, 4471, 952, 953, 402, 2475, 403, 404, 4758, 955, 2437, 4139, 1482, 3602, 4434, 3721, 1592, 1825, 1698, 1519, 1352, 4202, 4409, 1977, 1917, 405, 406, 407, 408, 956, 2272, 957, 2267, 1631, 1684, 409, 2054, 3889, 958, 410, 2117, 411, 959, 412, 960, 2443, 4712, 4619, 2285, 414, 415, 961, 2352, 2417, 962, 1905, 416, 963, 417, 418, 3675, 1833, 3974, 419, 964, 421, 965, 966, 967, 968, 2365, 969, 970, 422, 1897, 2056, 2309, 4733, 1272, 4754, 2207, 971, 423, 4133, 3830, 4865, 972, 424, 973, 425, 2068, 4955, 426, 974, 427, 1708, 4676, 428, 4141, 430, 3749, 431, 1619, 975, 4044, 3590, 1944, 4126, 1923, 1706, 432, 976, 977, 978, 4867, 4400, 433, 434, 1606, 2143, 979, 435, 980, 2604, 2895, 981, 436, 3448, 4959, 2188, 437, 438, 1692, 982, 2388, 2433, 2243, 983, 2035, 2120, 4940, 439, 440, 2474, 442, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 453, 992, 1728, 993, 4703, 994, 995, 454, 1696, 996, 2039, 455, 997, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 467, 468, 469, 1231, 1468, 470, 471, 2496, 472, 473, 1005, 474, 475, 2108, 4725, 476, 1273, 1007, 1744, 1488, 1659, 477, 478, 4598, 1604, 4753, 2493, 479, 1008, 1009, 480, 1011, 1012, 1013, 1014, 481, 4313, 482, 483, 1499, 484, 1015, 4761, 2091, 2513, 1016, 4073, 1777, 2172, 485, 2205, 4592, 4545, 486, 1458, 4274, 2122, 4905, 5003, 4599, 4796, 2152, 4539, 4528, 4904, 4842, 4986, 4846, 4650, 4595, 2195, 4681, 2142, 487, 4710, 1775, 4205, 1526, 4586, 4602, 4518, 1697, 1017, 2000, 4692, 4795, 2480, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 2202, 5001, 2136, 493, 2211, 4532, 494, 2421, 495, 4090, 2378, 2104, 2210, 2247, 2478, 2047, 496, 4620, 4978, 4614, 1019, 2012, 1803, 3642, 497, 1322, 1020, 498, 1021, 2235, 2302, 2488, 4665, 4540, 5006, 499, 2049, 4685, 500, 501, 1022, 502, 1333, 503, 1023, 4686, 4804, 504, 4727, 505, 506, 4938, 4805, 2065, 1890, 4891, 1024, 507, 508, 509, 1025, 510, 1026, 3608, 1732, 511, 2397, 1864, 1816, 1531, 512, 513, 2451, 3636, 5012, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 2093, 4746, 2489, 4922, 2145, 1030, 4832, 4950, 1261, 4027, 3478, 3763, 2959, 1293, 2462, 517, 3197, 1595, 3413, 2238, 1031, 2228, 518, 2057, 2305, 2133, 2061, 4526, 4617, 1033, 2429, 2311, 1675, 4785, 1797, 519, 4578, 2271, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 535, 1038, 536, 1039, 537, 538, 539, 1040, 3767, 1929, 1042, 3683, 2100, 4853, 2300, 2165, 540, 541, 1043, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 1044, 3720, 4351, 4414, 1673, 3453, 3254, 2501, 1649, 543, 544, 545, 546, 1045, 547, 4240, 3580, 4680, 1046, 548, 2062, 1047, 550, 2598, 551, 552, 553, 3819, 1259, 4062, 1048, 554, 2841, 1049, 1050, 3515, 2219, 1433, 1323, 4301, 555, 556, 1051, 557, 558, 1052, 1053, 3738, 1567, 559, 1054, 1270, 3857, 1055, 2151, 560, 2175, 2089, 1056, 2198, 2193, 1257, 1238, 3662, 561, 2186, 1057, 4637, 562, 1058, 563, 4872, 2097, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2255, 2113, 590, 591, 1085, 592, 2308, 593, 594, 2274, 595, 4931, 2407, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 1086, 601, 4017, 4477, 1794, 2483, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 610, 611, 1093, 612, 1094, 3701, 4389, 613, 4897, 1603, 614, 1095, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4801, 2398, 2239, 1101, 1667, 1384, 623, 4745, 3700, 1788, 1600, 624, 2098, 625, 4550, 4721, 4960, 4878, 2473, 4792, 4675, 1102, 1423, 626, 2771, 627, 1339, 3875, 628, 629, 3070, 4565, 2118, 4818, 2557, 2795, 2906, 2103, 4869, 2029, 3299, 3118, 4932, 4838, 4901, 4657, 4646, 4875, 4627, 4515, 4876, 4965, 4605, 4576, 2028, 4985, 4850, 1103, 4747, 1614, 1947, 2237, 630, 631, 4618, 4479, 3766, 632, 1104, 1105, 4886, 1106, 2432, 633, 1107, 4530, 634, 2371, 4651, 1108, 4536, 635, 1813, 636, 2431, 4866, 4991, 4814, 4560, 4956, 637, 4555, 1109, 4941, 4577, 2213, 638, 639, 3706, 1110, 640, 1111, 641, 4581, 4893, 4831, 2481, 4644, 4734, 2190, 4524, 4881, 4837, 4888, 4648, 642, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4760, 1115, 4918, 4688, 3920, 644, 4723, 4949, 4748, 4607, 4719, 1116, 645, 646, 4611, 2353, 4645, 4998, 2307, 647, 1801, 4279, 3762, 1637, 1509, 4843, 4797, 4590, 4995, 4541, 2200, 4580, 4858, 648, 649, 2502, 1117, 4780, 4800, 4551, 4674, 1118, 1119, 2965, 2369, 2695, 3293, 650, 651, 4730, 4862, 4722, 4944, 1120, 652, 1121, 4983, 4784, 4877, 4643, 653, 654, 4909, 1122, 3264, 655, 2076, 4849, 656, 657, 2447, 658, 659, 4035, 2910, 3391, 2808, 3099, 2913, 3676, 2852, 3284, 2731, 1609, 2375, 2201, 3537, 3994, 1319, 4467, 1802, 1227, 1623, 660, 4844, 2086, 4770, 2192, 1123, 661, 2364, 662, 663, 664, 1124, 1125, 665, 2487, 4899, 2138, 1126, 666, 667, 4569, 4554, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 2456, 4606, 4910, 2123, 1136, 1137, 2358, 5005, 2208, 4952, 4945, 1933, 1731, 1793, 675, 676, 3342, 1138, 677, 4597, 4870, 1140, 4990, 4954, 4916, 678, 1141, 4737, 4527, 2253, 4531, 679, 1142, 1143, 680, 4771, 1144, 4638, 681, 4705, 4525, 4522, 4898, 2325, 2415, 5004, 1145, 682, 683, 1146, 684, 685, 2445, 686, 687, 4861, 2157, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4793, 4575, 2160, 4828, 4731, 4718, 4802, 4750, 1150, 2081, 2258, 697, 1795, 1372, 1695, 1866, 1151, 4115, 1719, 2280, 4662, 698, 2176, 2194, 2217, 1920, 699, 700, 701, 4999, 1544, 4739, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 4824, 705, 2340, 4182, 706, 2442, 2484, 707, 708, 709, 4667, 1153, 2412, 4476, 2053, 710, 1375, 711, 712, 713, 4829, 4738, 4811, 4570, 4961, 1245, 714, 2045, 4707, 715, 2169, 4713, 1841, 4234, 4924, 1154, 1314, 4096, 2023, 716, 717, 4895, 718, 1155, 719, 720, 2063, 1311, 721, 4642, 722, 4885, 4666, 2249, 4217, 1156, 723, 1157, 1627, 2116, 4566, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 730, 731, 2024, 1846, 2507, 2368, 2019, 2333, 3815, 2005, 2266, 1964, 2347, 2385, 732, 2177, 2059, 4958, 1822, 4815, 1262, 2094, 4480, 1537, 1291, 2419, 1318, 2283, 4976, 4294, 2486, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4679, 4835, 2259, 4839, 4571, 1166, 4968, 2250, 4912, 2423, 737, 1167, 4989, 4684, 4669, 4552, 4612, 2127, 1745, 1973, 4767, 1168, 2273, 2310, 3807, 4307, 1169, 2303, 1454, or 1781.

In some aspects, X³, X⁶, and X¹ of SEQ ID NO: 5014 are basic amino acid residues. In some aspects, X³, X⁶, and X⁴ of SEQ ID NO: 5014 are basic amino acid residues. In some aspects, X³, X⁶, X¹, and X⁴ of SEQ ID NO: 5014 are basic amino acid residues.

In various aspects, the present disclosure provides a viral protein (VP) capsid polypeptide comprising a 581 to 589 region corresponding to residues 581 to 589 of an AAV5 VP1 polypeptide, wherein the 581 to 589 region of the VP capsid polypeptide comprises at least two basic amino acid residues and no more than one acidic amino acid residue.

In some aspects, the VP capsid polypeptide comprises one or more features selected from the group consisting of: (a) the 581 to 589 region comprises two or more basic amino acid residues, (b) X³, X⁶, X¹, or X⁴ of SEQ ID NO: 5014, or any combination thereof are basic amino acid residues, and (c) the 581 to 589 region comprises no more than one acidic amino acid residue.

In some aspects, the VP capsid polypeptide comprises two or more of the features. In some aspects, the VP capsid polypeptide comprises three of the features. In some aspects, the acidic amino acid residue is D or E. In some aspects, the basic amino acid residues are K, R, or H.

In various aspects, the present disclosure provides a viral protein (VP) capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 14-SEQ ID NO: 2013.

In some aspects, the 581 to 589 region comprises a sequence of any one of SEQ ID NO: 14-SEQ ID NO: 2013.

In various aspects, the present disclosure provides a viral protein (VP) capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 2514-SEQ ID NO: 4513.

In some aspects, the 581 to 589 region comprises a sequence of any one of SEQ ID NO: 2514-SEQ ID NO: 4513. In some aspects, the 581 to 589 region confers increased retina tissue tropism compared to a wild type AAV5 VP capsid polypeptide of SEQ ID NO: 1. In some aspects, the retina tissue tropism is at least 1.5-fold, at least 2-fold, at least 2.5-fold, at least 3-fold, at least 3.5-fold, at least 4-fold, at least 5-fold, at least 10-fold, at least 20-fold, at least 50-fold, at least 75-fold, at least 100-fold, at least 200-fold, or at least 500-fold higher than the wild type AAV5 VP capsid polypeptide of SEQ ID NO: 1.

In some aspects, the 581 to 589 region further confers increased retinal pigment epithelium tissue tropism compared to a wild type AAV5 VP capsid polypeptide of SEQ ID NO: 1. In some aspects, the 581 to 589 region confers decreased photoreceptor tissue-tropism compared to a wild type AAV5 VP capsid polypeptide of SEQ ID NO: 1. In some aspects, the photoreceptor tissue comprises rod photoreceptor cells, cone photoreceptor cells, or a combination thereof. In some aspects, the VP capsid polypeptide comprises an amino acid sequence at least 70%, at least 80%, at least 85%, at least 90%, at least 95%, at least 97%, at least 98%, at least 98.5%, at least 99%, or at least 99.5% identical to SEQ ID NO: 1.

In some aspects, the VP capsid polypeptide has a sequence of SEQ ID NO: 2, wherein residues 581 to 589 of SEQ ID NO: 2 (X¹X²X³X⁴X⁵X⁶X⁷X⁸X⁹) correspond to the 581 to 589 region. In some aspects, the VP capsid polypeptide is capable of assembling into a recombinant viral capsid. In some aspects, the VP capsid polypeptide comprises at least one amino acid substitution as compared to each of SEQ ID NO: 3, SEQ ID NO: 4, SEQ ID NO: 5, SEQ ID NO: 6, SEQ ID NO: 7, and SEQ ID NO: 8. In some aspects, the 581 to 589 region comprises a net positive charge at pH 7.4.

In some aspects, the VP capsid polypeptide further comprises one or more mutations outside of the 581 to 589 region relative to a wild type VP capsid polypeptide of SEQ ID NO: 1. In some aspects, the 581 to 589 region confers on a recombinant viral capsid assembled from the VP capsid polypeptide improved stability compared to a wild type AAV capsid comprising a peptide of SEQ ID NO: 1. In some aspects, the 581 to 589 region confers lower toxicity upon administration to a subject compared to a wild type VP capsid polypeptide of SEQ ID NO: 1. In some aspects, the VP capsid polypeptide further comprises one or more mutations outside of the 581 to 589 region that contributes to reduced production of neutralizing antibodies relative to a wild type VP capsid polypeptide of SEQ ID NO: 1. In some aspects, the VP capsid polypeptide further comprises one or more mutations outside of the 581 to 589 region that improves manufacturability relative to a wild type VP capsid polypeptide of SEQ ID NO: 1.

In various aspects, the present disclosure provides a recombinant viral adeno-associated virus (rAAV) capsid comprising a VP capsid polypeptide as described herein, wherein the rAAV capsid preferentially targets retina tissue.

In some aspects, the rAAv further comprises a VP2 polypeptide comprising the 581 to 589 region and a VP3 polypeptide comprising the 581 to 589 region.

In various aspects, the present disclosure provides a recombinant adeno-associated virus (rAAV), comprising a VP capsid polypeptide as described herein assembled into a recombinant viral capsid and a payload encapsidated by the recombinant viral capsid.

In some aspects, the rAAV preferentially targets retina tissue. In some aspects, the rAAV further comprises a VP2 polypeptide comprising the 581 to 589 region and a VP3 polypeptide comprising the 581 to 589 region. In some aspects, the payload encodes a therapeutic polynucleotide or a therapeutic peptide. In some aspects, the payload encodes a guide RNA, a tRNA, a suppressor tRNA, a siRNA, a miRNA, an mRNA, a shRNA, a circular RNA, or an antisense oligonucleotide (ASO), a ribozyme, a DNAzyme, an aptamer, or any combination thereof. In some aspects, the guide RNA is a CRISPR/Cas guide RNA or an ADAR guide RNA. In some aspects, the payload encodes a component of a CRISPR/Cas system, an adenosine deaminase acting on RNA (ADAR) enzyme, a transcriptional activator, or a transcriptional repressor.

In various aspects, the present disclosure provides a pharmaceutical composition comprising a VP capsid polypeptide as described herein, a rAAV capsid as described herein, or a rAAV as described herein.

In various aspects, the present disclosure provides a method of delivering a payload to a retina tissue of a subject, the method comprising administering a recombinant adeno-associated virus (rAAV) to the subject via intravitreal administration, wherein the rAAV encapsidates the payload, and wherein the rAAV comprises a VP capsid polypeptide comprising a 581 to 589 region comprising at least one substitution relative to SEQ ID NO: 9, and wherein the 581 to 589 region comprises one or more basic amino acid residues.

In various aspects, the present disclosure provides a method of delivering a payload to a retina tissue of a subject, the method comprising administering a recombinant adeno-associated virus (rAAV) to the subject via intravitreal administration, wherein the rAAV encapsidates the payload, and wherein the rAAV comprises a VP capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 14-SEQ ID NO: 2013 or SEQ ID NO: 2514-SEQ ID NO: 4513.

In various aspects, the present disclosure provides a method of delivering a payload to a retina tissue of a subject, the method comprising administering a recombinant adeno-associated virus (rAAV) to the subject via intravitreal administration, wherein the rAAV encapsidates the payload, and wherein the rAAV comprises a VP capsid polypeptide as described herein.

In various aspects, the present disclosure provides a method of delivering a payload to a retina tissue of a subject, the method comprising: (i) administering a recombinant adeno-associated virus (rAAV) to the subject, wherein the rAAV encapsidates the payload, and wherein the rAAV comprises a VP capsid polypeptide comprising a 581 to 589 region comprising at least one substitution relative to SEQ ID NO: 9, and wherein the 581 to 589 region comprises one or more basic amino acid residues; and (ii) delivering the payload to the retina tissue.

In various aspects, the present disclosure provides a method of delivering a payload to a retina tissue of a subject, the method comprising: (i) administering a recombinant adeno-associated virus (rAAV) to the subject, wherein the rAAV encapsidates the payload, and wherein the rAAV comprises a VP capsid polypeptide comprising a 581 to 589 region having at least 85% sequence identity to any one of SEQ ID NO: 14-SEQ ID NO: 2013 or SEQ ID NO: 2514-SEQ ID NO: 4513; and (ii) delivering the payload to the retina tissue.

In various aspects, the present disclosure provides a method of delivering a payload to a retina tissue of a subject, the method comprising: (i) administering a recombinant adeno-associated virus (rAAV) to the subject, wherein the rAAV encapsidates the payload, and wherein the rAAV comprises a VP capsid polypeptide as described herein; and (ii) delivering the payload to the retina tissue.

In some aspects, the method comprises administering the rAAV via intravitreal administration. In some aspects, the method comprises administering the rAAV via subretinal injection, topical mucosal administration, or systemic administration. In some aspects, the method further comprises expressing a therapeutic polypeptide or a therapeutic polynucleotide encoded by the payload. In some aspects, the method further comprises producing a therapeutic effect upon expression of the payload. In some aspects, the therapeutic effect is produced upon administration of from 1×10⁵ to 5×10¹⁴ rAAVs. In some aspects, the method comprises administering an amount of the rAAV sufficient to produce the therapeutic effect without producing a toxicity in the subject.

In various aspects, the present disclosure provides a method of treating a condition in a subject, the method comprising administering a recombinant adeno-associated virus (rAAV) to the subject via intravitreal administration, wherein the rAAV encapsidates a payload, and wherein the rAAV comprises a VP capsid polypeptide comprising a 581 to 589 region comprising at least one substitution relative to SEQ ID NO: 9, and wherein the 581 to 589 region comprises one or more basic amino acid residues.

In various aspects, the present disclosure provides a method of treating a condition in a subject, the method comprising administering a recombinant adeno-associated virus (rAAV) to the subject via intravitreal administration, wherein the rAAV encapsidates a payload, and wherein the rAAV comprises a VP capsid polypeptide comprising a 581 to 589 region corresponding to residues 581 to 589 of an AAV5 VP1 polypeptide having at least 85% sequence identity to any one of SEQ ID NO: 14-SEQ ID NO: 2013 or SEQ ID NO: 2514-SEQ ID NO: 4513.

In various aspects, the present disclosure provides a method of treating a condition in a subject, the method comprising administering a recombinant adeno-associated virus (rAAV) to the subject via intravitreal administration, wherein the rAAV encapsidates a payload, and wherein the rAAV comprises a VP capsid polypeptide as described herein.

In some aspects, the method further comprises expressing the payload in a retina tissue of the subject. In some aspects, the method further comprises producing a therapeutic effect in the subject.

In various aspects, the present disclosure provides a method of treating a condition in a subject, the method comprising: (i) administering a recombinant adeno-associated virus (rAAV) to the subject, wherein the rAAV encapsidates a payload, and wherein the rAAV comprises a VP capsid polypeptide comprising a 581 to 589 region comprising at least one substitution relative to SEQ ID NO: 9, and wherein the 581 to 589 region comprises one or more basic amino acid residues; and (ii) expressing the payload in a retina tissue of the subject, thereby treating the condition in the subject.

In various aspects, the present disclosure provides a method of treating a condition in a subject, the method comprising: (i) administering a recombinant adeno-associated virus (rAAV) to the subject, wherein the rAAV encapsidates a payload, and wherein the rAAV comprises a VP capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 14-SEQ ID NO: 2013 or SEQ ID NO: 2514-SEQ ID NO: 4513; and (ii) expressing the payload in a retina tissue of the subject, thereby treating the condition in the subject.

In various aspects, the present disclosure provides a method of treating a condition in a subject, the method comprising: (i) administering a recombinant adeno-associated virus (rAAV) to the subject, wherein the rAAV encapsidates a payload, and wherein the rAAV comprises a VP capsid polypeptide as described herein; and (ii) expressing the payload in a retina tissue of the subject, thereby treating the condition in the subject.

In various aspects, the present disclosure provides a method of treating a condition in a subject, the method comprising: (i) administering a recombinant adeno-associated virus (rAAV) to the subject, wherein the rAAV encapsidates a payload, and wherein the rAAV comprises a VP capsid polypeptide as described herein; (ii) delivering the payload to a retina tissue of the subject; and (iii) producing a therapeutic effect in the retina tissue, thereby treating the condition.

In some aspects, the method comprises administering the rAAV via intravitreal administration. In some aspects, the method comprises administering the rAAV via subretinal injection, topical mucosal administration, or systemic administration. In some aspects, the condition is an ocular condition. In some aspects, the ocular condition is achromatopsia, macular degeneration, cataracts, choroideremia, glaucoma, optical neuropathy, Marfan syndrome, myopia, polypoidal choroidal vasculopathy, retinitis pigmentosa, Stargardt disease, Usher syndrome, Leber congenital amaurosis, Leber hereditary optical neuropathy, Uveal melanoma, or X-linked retinoschisis. In some aspects, the ocular condition is Stargardt disease.

In some aspects, the method further comprises delivering the rAAV to a retina tissue with higher tissue tropism than a wild type AAV5 capsid comprising a peptide of SEQ ID NO: 1. In some aspects, the method further comprises delivering the rAAV to a retina tissue with higher tissue tropism than for photoreceptor tissue. In some aspects, the method comprises delivering the rAAV to a retina tissue with at least 2.5-fold, at least 3-fold, at least 3.5-fold, at least 4-fold, at least 5-fold, at least 10-fold, at least 20-fold, at least 50-fold, at least 75-fold, at least 100-fold, at least 200-fold, at least 500-fold higher, or at least 1000-fold higher tissue tropism than a wild type AAV5 capsid comprising a peptide of SEQ ID NO: 1. In some aspects, the method comprises administering from 1×10⁵ to 5×10¹⁴ rAAVs.

In some aspects, the payload encodes a therapeutic protein, therapeutic polynucleotide, a guide RNA, a tRNA, a suppressor tRNA, a siRNA, a miRNA, an mRNA, a shRNA, a circular RNA, an antisense oligonucleotide (ASO), a ribozyme, a DNAzyme, an aptamer, or any combination thereof. In some aspects, the guide RNA is a CRISPR/Cas guide RNA or an ADAR guide RNA. In some aspects, the therapeutic protein is selected from the group consisting of CNGB3, NOS2A, CFH, CF, C2, C3, CFB, HTRA1/LOC, MMP-9, TIMP-3, SLC16A8, GEMIN4, CYP51A1, RIC1, TAPT1, TAF1A, WDR87, APE1, MIP, Cx50, GJA3, GJA8, CRYAA, CRYBB2, PRX, POLR3B, XRCC1, ZNF350, EPHA2, REP1, CALM2, MPP-7, Optineurin, LOX1, CYP1B1, CAV1/2, MYOC, PITX2, FOXC1, PAX6, LTBP2, Complex I, ND, OPA1, RPE65, FBN1, TGFBR2, MTHFR, MTR, MTRR, HGF, C-MET, UMODL1, MMP-1/2, CBS, IGF-1, UHRF1BP1L, PTPRR, PPFIA2, P4HA2, SERPING1, PEDF, ARMS2-HTRA1, FGD6, ABCG1, LOC387715, CETP, NRL, RDH12, PRPH2 (RDS), RHO, RPGR, SNRNP200, NR2E3, IMPDH1, CRX, HK1, IMPDH2, PRPF3, AGBL5, ABCA1, ABCA4, CRB1, USH2A, NRP1, ND4, RLBP1, PTEN, BAP1, GNAQ, GNA11, DDEF1, SF3B1, EIF1AX, CDKN2A, p14ARF, HERC2/OCA2, VEGF, and RS1. In some aspects, the therapeutic polynucleotide targets an mRNA encoding a protein selected from the group consisting of CNGB3, NOS2A, CFH, CF, C2, C3, CFB, HTRA1/LOC, MMP-9, TIMP-3, SLC16A8, GEMIN4, CYP51A1, RIC1, TAPT1, TAF1A, WDR87, APE1, MIP, Cx50, GJA3, GJA8, CRYAA, CRYBB2, PRX, POLR3B, XRCC1, ZNF350, EPHA2, REP1, CALM2, MPP-7, Optineurin, LOX1, CYP1B1, CAV1/2, MYOC, PITX2, FOXC1, PAX6, LTBP2, Complex I, ND, OPA1, RPE65, FBN1, TGFBR2, MTHFR, MTR, MTRR, HGF, C-MET, UMODL1, MMP-1/2, CBS, IGF-1, UHRF1BP1L, PTPRR, PPFIA2, P4HA2, SERPING1, PEDF, ARMS2-HTRA1, FGD6, ABCG1, LOC387715, CETP, NRL, RDH12, PRPH2 (RDS), RHO, RPGR, SNRNP200, NR2E3, IMPDH1, CRX, HK1, IMPDH2, PRPF3, AGBL5, ABCA1, ABCA4, CRB1, USH2A, NRP1, ND4, RLBP1, PTEN, BAP1, GNAQ, GNA11, DDEF1, SF3B1, EIF1AX, CDKN2A, p14ARF, HERC2/OCA2, VEGF, or RS1. In some aspects, the payload encodes a therapeutic polynucleotide targeting ABCA1, ABCA4, or CRB1 or a transgene encoding ABCA1, ABCA4, or CRB1. In some aspects, the payload encodes a component of a CRISPR/Cas system, an adenosine deaminase acting on RNA (ADAR) enzyme, a transcriptional activator, or a transcriptional repressor. In some aspects, the component of the CRISPR/Cas system comprises a Cas3, a Cas8, a Cas10, a Cas9, a Cas4, a Cas12, a Cas13, a guide RNA, or a combination thereof. In some aspects, the ADAR enzyme is ADAR1 or ADAR2. In some aspects, the transcriptional activator is VP64. In some aspects, the transcriptional repressor is KRAB.

In some aspects, the method further comprises expressing a therapeutic polypeptide or a therapeutic polynucleotide encoded by the payload. In some aspects, the method comprises expressing the therapeutic polypeptide or the therapeutic polynucleotide at a higher level in a retina tissue compared to a wild type AAV5 capsid comprising a peptide of SEQ ID NO: 1. In some aspects, the method comprises expressing the therapeutic polypeptide or the therapeutic polynucleotide in a retina tissue at a level that is at least 3-fold, at least 3.5-fold, at least 4-fold, at least 5-fold, at least 10-fold, at least 20-fold, at least 50-fold, at least 75-fold, at least 100-fold, at least 200-fold, at least 500-fold higher, or at least 1000-fold higher compared to the wild type AAV5 capsid. In some aspects, the method comprises producing a toxicity in the subject that is lower than a toxicity produced upon administration of a comparable number of wild type AAV5 capsids comprising a VP capsid polypeptide of SEQ ID NO: 1. In some aspects, the method comprises producing a toxicity in the subject that is lower than a toxicity produced upon administration of a number of wild type AAV5 capsids comprising a VP capsid polypeptide of SEQ ID NO: 1 sufficient to deliver a comparable number of payloads to a retina tissue. In some aspects, the method comprises producing a concentration of neutralizing antibodies in the subject that is lower than a concentration of neutralizing antibodies produced upon administration of a comparable number of wild type AAV5 capsids comprising a VP capsid polypeptide of SEQ ID NO: 1. In some aspects, the method comprises producing a concentration of neutralizing antibodies in the subject that is lower than a concentration of neutralizing antibodies produced upon administration of a comparable number of wild type AAV2 capsids. In some aspects, the method comprises producing a concentration of neutralizing antibodies in the subject that is lower than a concentration of neutralizing antibodies produced upon administration of a comparable number of wild type AAV8 capsids. In some aspects, the method comprises producing a concentration of neutralizing antibodies in the subject that is lower than a concentration of neutralizing antibodies produced upon administration of a comparable number of wild type AAV9 capsids. In some aspects, the method comprises producing a concentration of neutralizing antibodies in the subject that is lower than a concentration of neutralizing antibodies produced upon administration of a number of wild type AAV5 capsids comprising a VP capsid polypeptide of SEQ ID NO: 1 sufficient to deliver a comparable number of payloads to a retina tissue.

In various aspects, the present disclosure provides a rAAV capsid as described herein, a rAAV as described herein, or a pharmaceutical composition as described herein for use as a medicament.

In various aspects, the present disclosure provides a rAAV capsid as described herein, a rAAV as described herein, or a pharmaceutical composition as described herein for use in a method of treating an ocular condition.

In some aspects, the ocular condition is achromatopsia, macular degeneration, cataracts, choroideremia, glaucoma, optical neuropathy, Marfan syndrome, myopia, polypoidal choroidal vasculopathy, retinitis pigmentosa, Stargardt disease, Usher syndrome, Leber congenital amaurosis, Leber hereditary optical neuropathy, Uveal melanoma, or X-linked retinoschisis. In some aspects, the ocular condition is Stargardt disease.

In various aspects, the present disclosure provides a viral protein (VP) capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 2014-SEQ ID NO: 2513.

In some aspects, the 581 to 589 region comprises a sequence of any one of SEQ ID NO: 2014-SEQ ID NO: 2513.

In various aspects, the present disclosure provides a viral protein (VP) capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 4514-SEQ ID NO: 5013.

In some aspects, the 581 to 589 region comprises a sequence of any one of SEQ ID NO: 4514-SEQ ID NO: 5013.

In various aspects, the present disclosure provides a recombinant viral adeno-associated virus (rAAV) capsid comprising a VP capsid polypeptide as described herein, wherein the rAAV capsid preferentially targets retinal pigment epithelium tissue.

In various aspects, the present disclosure provides a recombinant viral adeno-associated virus (rAAV) capsid comprising a VP capsid polypeptide as described herein, wherein the rAAV capsid preferentially targets choroid tissue.

In various aspects, the present disclosure provides a method of delivering a payload to a retinal pigment epithelium tissue of a subject, the method comprising administering a recombinant adeno-associated virus (rAAV) to the subject via intravitreal administration, wherein the rAAV encapsidates the payload, and wherein the rAAV comprises a VP capsid polypeptide comprising a 581 to 589 region corresponding to residues 581 to 589 of an AAV5 VP1 polypeptide having at least 85% sequence identity to any one of SEQ ID NO: 2014-SEQ ID NO: 2513 or SEQ ID NO: 4514-SEQ ID NO: 5013.

In various aspects, the present disclosure provides a method of delivering a payload to a retinal pigment epithelium tissue of a subject, the method comprising administering a recombinant adeno-associated virus (rAAV) to the subject via intravitreal administration, wherein the rAAV encapsidates the payload, and wherein the rAAV comprises a VP capsid polypeptide comprising a 581 to 589 region corresponding to residues 581 to 589 of an AAV5 VP1 polypeptide having at least 85% sequence identity to any one of SEQ ID NO: 2014-SEQ ID NO: 2513 or SEQ ID NO: 4514-SEQ ID NO: 5013.

In various aspects, the present disclosure provides a method of delivering a payload to a choroid tissue of a subject, the method comprising: (i) administering a recombinant adeno-associated virus (rAAV) to the subject, wherein the rAAV encapsidates the payload, and wherein the rAAV comprises a VP capsid polypeptide comprising a 581 to 589 region having at least 85% sequence identity to any one of SEQ ID NO: 2014-SEQ ID NO: 2513 or SEQ ID NO: 4514-SEQ ID NO: 5013; and (ii) delivering the payload to the choroid tissue.

In various aspects, the present disclosure provides a method of delivering a payload to a retinal pigment epithelium tissue of a subject, the method comprising: (i) administering a recombinant adeno-associated virus (rAAV) to the subject, wherein the rAAV encapsidates the payload, and wherein the rAAV comprises a VP capsid polypeptide comprising a 581 to 589 region having at least 85% sequence identity to any one of SEQ ID NO: 2014-SEQ ID NO: 2513 or SEQ ID NO: 4514-SEQ ID NO: 5013; and (ii) delivering the payload to the retinal pigment epithelium tissue.

In various aspects, the present disclosure provides a method of treating a condition in a subject, the method comprising administering a recombinant adeno-associated virus (rAAV) to the subject via intravitreal administration, wherein the rAAV encapsidates a payload, and wherein the rAAV comprises a VP capsid polypeptide comprising a 581 to 589 region corresponding to residues 581 to 589 of an AAV5 VP1 polypeptide having at least 85% sequence identity to any one of SEQ ID NO: 2014-SEQ ID NO: 2513 or SEQ ID NO: 4514-SEQ ID NO: 5013.

In various aspects, the present disclosure provides a method of treating a condition in a subject, the method comprising: (i) administering a recombinant adeno-associated virus (rAAV) to the subject, wherein the rAAV encapsidates a payload, and wherein the rAAV comprises a VP capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 2014-SEQ ID NO: 2513 or SEQ ID NO: 4514-SEQ ID NO: 5013; and (ii) expressing the payload in a retinal pigment epithelium tissue of the subject, thereby treating the condition in the subject.

### INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### BRIEF DESCRIPTION OF THE DRAWINGS

These and other features, aspects, and advantages of the present invention will become better understood with regard to the following description, and accompanying drawings where:

FIG. 1 is a schematic of the high throughput AAV capsid engineering system for identification of tissue-tropic sequences for intravitreal administration. A capsid library, selected for the ability to assemble into viral capsids, is administered to a non-human primate (NHP) by intravitreal administration. Tissues, including eye tissues such as retina, optic nerve, choroid, aqueous humor, and vitreous humor, are recovered, and the capsids accumulated in each tissue are sequenced. The resulting sequencing data are fed into a machine learning-powered bioinformatics pipeline to identify viral capsid polypeptide sequences and biophysical properties that promote tissue-specific transduction/infection.

FIG. 2A provides a side view (top panel) and top view (bottom panel) of key residues of known AAV capsids that have been shown to interact with target cells.

FIG. 2B illustrates salient features of the AAV capsid library described in EXAMPLE 1, showing the region of introduced diversity, with residue numbering corresponding to the numbering of amino acids in AAV5 VP1.

FIG. 3 is a bar graph showing viral genomes per milligram of tissue recovered from various eye tissues following NHP capsid library screening performed as illustrated in FIG. 1. Screening was performed independently in two non-human primates (“NHP1” and “NHP2”).

FIG. 4 is a bar graph showing the number of unique variant capsid polypeptide sequences (“#of varAA”) identified within various tissues from two NHPs (“NHP #1” and “NHP #2”) administered a recombinant AAV library via intravitreal injection.

FIG. 5A-FIG. 5D are sequence logo plots depicting relative amino acid enrichment and depletion at each amino acid position of the 581 to 589 region in the retina from two NHPs administered a recombinant AAV library via intravitreal injection. FIG. 5A illustrates enrichment in retina versus other remaining eye tissues (“other”) in example NHP1. FIG. 5B illustrates enrichment in retina versus other remaining eye tissues (“other”) in a second example NHP2. FIG. 5C illustrates enrichment in retina versus vitreous/aqueous humor (“humor”) in a first NHP. FIG. 5D illustrates enrichment in retina versus vitreous/aqueous humor (“humor”) in a second NHP.

FIG. 6A and FIG. 6B are heatmaps showing relative amino acid enrichment in retina at each amino acid position of the 581 to 589 region (corresponding to positions 581-589 in the VP1 sequence) for a first (FIG. 6A) and second (FIG. 6B) NHP, administered a recombinant AAV library via intravitreal injection. The enrichment shown is relative to “ST2”, the diversity of the assembled capsid library pre-infusion.

FIG. 7 is a bar graph showing numbers of AAV capsid polypeptide variants observed in a primary screen of an engineered AAV capsid library. Counts are provided for the total number of variants found in either retina or RPE tissue (“total”), the number of variants found in only that tissue (“tissue_unique”), the number of tissue-unique variants found in more than one non-human primate (“multiple_nhps”), and the number of tissue-unique variants found in more than one replicate (“multiple_reps,” includes variants found in multiple NHPs).

FIG. 8 is a violin plot comparing the predictive probability (“retina_score”) of having retina-specific tropism for retina tissue-unique variants separated based on numbers of observations, either a single observation (“n”) or multiple observations (“y”), within non-human primates (“multiple_nhps”) or within all replicates (“multiple_reps”).

FIG. 9 is a box and whisker plot showing the predictive probability (“Average ML Score”) of having retina-specific tropism for all retina tissue-unique variants identified from the primary screen (“observed”) or generated through machine learning models (“synthetic”). Subsets of observed retina variants found in multiple NHPs (“Multiple NHPs”) or multiple replicates (“Multiple Replicates”) are noted, along with synthetic variants generated using three distinct modeling strategies (“Input optimization”, “Sample PWM”, and “Sample uniform”).

FIG. 10 is plot showing the predictive probability (“Average ML Score”) of having retina-specific tropism for retina tissue-unique variants selected for inclusion in the secondary screen that were identified from the primary screen (“observed”) or through machine learning models (“synthetic”).

### DETAILED DESCRIPTION OF THE INVENTION

Unless described otherwise, all technical and scientific terms used herein have the meaning commonly understood by one of ordinary skill in the art to which the invention pertains.

Unless otherwise stated, whenever a range is recited, the range is inclusive of the recited endpoints. For example, the region from amino acid residue 581 to amino acid residue 589 of SEQ ID NO: 1 includes amino acid residues 581 and 589.

“Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 77.7%, 80%, 88.8%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed. The term percent “identity” or percent “homology,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. For purposes herein, determination of percent identity and sequence similarity is performed using the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/). For example, where an amino acid sequence consisting of 9 residues is compared with another 9 residue amino acid sequence, if 8 residues match then the sequences have 88.8% identity, if 7 residues match then the sequences have 77.7% identity.

In some cases, the identity between a reference sequence (query sequence, e.g., a sequence of the disclosure) and a subject sequence, also referred to as a global sequence alignment, can be determined using the FASTDB computer program. In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

Peptide sequences for use in the present invention may comprises one or more substitutions, i.e. amino acid substitutions. The substitutions may be conservative amino acid substitutions. Therefore, peptide sequences for use in the present invention may include one or more conservative amino acid substitutions, such that the resulting sequence has a similar amino acid sequence and/or retains the same function. The skilled person is aware that various amino acids have similar biochemical properties and thus are “conservative”. One or more such amino acids of a protein, polypeptide or peptide can often be substituted by one or more other such amino acids without eliminating a desired activity of that protein, polypeptide or peptide. Thus, the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulfur containing side chains). It should be appreciated that amino acid substitutions within the scope of the present invention can be made using naturally occurring or non-naturally occurring amino acids. For example, the methyl group on an alanine may be replaced with an ethyl group, and/or minor changes may be made to the peptide backbone. Whether or not natural or synthetic amino acids are used, it is preferred that only L-amino acids are present. Substitutions of this nature are often referred to as “conservative substitutions”.

As used herein, “tropism” of a rAAV for a tissue may refer to the ability of a given rAAV to preferentially infect a given cell type or tissue. A degree of tropism may be determined by a ratio of an infection rate in a targeted tissue to an infection rate in a different, non-targeted tissue. As used herein, increased tropism for a given cell type or tissue, such as increased tropism conferred by a 581 to 589 region, is determined relative to a wild type AAV5 capsid. As used herein, “detargeting” of a rAAV to a tissue may refer to the ability of a given rAAV to avoid infecting a detargeted tissue or cell type while infecting one or more other tissues or cell types. A degree of detargeting may be determined by a ratio of an infection rate in a detargeted tissue to an infection rate of a different, non-detargeted tissue. As used herein, increased detargeting for a given cell type or tissue, such as increased detargeting conferred by a 581 to 589 region, is determined relative to a wild type AAV5 capsid.

As used herein, “tissue tropism” refers to a preference of a virus having an engineered VP capsid polypeptide of the present disclosure to infect a given tissue or be enriched in or accumulate in a given tissue. A “tissue-tropic” rAAV may specifically target or infect a first tissue or set of tissues and may not target or infect a second tissue or set of tissues. For example, a “retinal tissue-tropic” rAAV may specifically target or infect retinal tissue and may not target or infect vitreous humor, aqueous humor, or other tissues. A “tissue-detargeted” rAAV may specifically avoid targeting or avoid infection of the detargeted tissue or set of tissues while infecting a second tissue or set of tissues. For example, a “liver-detargeted” rAAV may not target or infect liver tissue but may infect one or more other tissues, such as ocular, nervous, muscle, skin, bone, and/or other tissue. In another example, a “photoreceptor-detargeted” rAAV may not target or infect photoreceptor cells but may infect one or more other cell or tissue types, such as aqueous humor, retina, retinal pigment epithelium, optic nerve, vitreous humor, and/or other tissue or cells. In another example, a “choroid-detargeted” rAAV may not target or infect choroid tissue but may infect one or more other tissue or cell types, such as aqueous humor, retina, retinal pigment epithelium, optic nerve, vitreous humor, and/or other tissue or cells. Tissue tropism or tissue detargeting, when used as a relative term and depending on the context in which it is described herein, refers to an increase or decrease in tissue tropism of a given rAAV virion having a first capsid polypeptide in a first tissue as compared to a second tissue and/or refers to an increase or decrease in tissue tropism of a given rAAV virion having a first capsid polypeptide to an rAAV virion having a second capsid polypeptide. In some embodiments, the first tissue can be a group of tissues. In some embodiments, the second tissue can be a group of tissues. For example, the first tissue may be retinal tissue and the second tissue may be a different eye tissue (e.g., aqueous humor, choroid, optic nerve, vitreous humor, or remaining eye tissue). In another example, tissue tropism can refer to cellular homing, such as targeting a retinal pigment epithelial cell, a photoreceptor cell (e.g., rod cells, cone cells, or a combination thereof), or both.

In some embodiments, the rAAV virions of the present disclosure may also be referred to as preferentially targeting a given tissue or having tissue selectivity for a given tissue. For example, an rAAV virion that preferentially targets retinal tissue may specifically target or infect retinal tissue and may not target or infect vitreous humor, aqueous humor, or other tissues. As another example, an rAAV virion that has retinal tissue selectivity may specifically target or infect retinal tissue and may not target or infect vitreous humor, aqueous humor, or other tissues.

For simplicity throughout this disclosure, viral capsid protein is generally referred to as “VP.” Viral capsid protein is referred to as VP1 when referencing AAV5 VP1 positional notation. In all cases, viral capsid sequences and mutations disclosed herein should be understood as pertaining to all isoforms of the capsid protein (VP1, VP2, and VP3), as a mixture of these isoforms assemble to form virions. The positional amino acid residue designations “581 to 589” are relative to the translational start of the VP1 polypeptide and should be adjusted accordingly to the relative start sites of VP2 and VP3. It should be understood that the present disclosure, when describing any particular VP1 sequence with mutations at particular amino acid residue positions, necessarily also encompasses corresponding mutations in VP2 and VP3. For example, any consensus sequence or specific sequence of a VP1 capsid protein having one or more mutations in the 581 to 589 region, corresponding to amino acid residues 581 to 589 of VP1, also encompasses VP2 and VP3 capsid proteins having said one or more mutations in an amino acid residue region in VP2 and VP3 corresponding to the amino acid residues of the VP1 581 to 589 region. For example, the amino acid residues of the 581 to 589 region of VP1 (SEQ ID NO: 1; MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPATGTYNLQEIVPGSVWMERDVYLQGPIWAKIPETGAHFHPSPAMGGFGLKHPPPM MLIKNTPVPGNITSFSDVPVSSFITQYSTGQVTVEMEWELKKENSKRWNPEIQYTNNYN DPQFVDFAPDSTGEYRTTRPIGTRYLTRPL) correspond to the amino acid residues of the 445 to 453 region of VP2 (SEQ ID NO: 10; TAPTGKRIDDHFPKRKKARTEEDSKPSTSSDAEAGPSGSQQLQIPAQPASSLGADTMSAG GGGPLGDNNQGADGVGNASGDWHCDSTWMGDRVVTKSTRTWVLPSYNNHQYREIKS GSVDGSNANAYFGYSTPWGYFDFNRFHSHWSPRDWQRLINNYWGFRPRSLRVKIFNIQ VKEVTVQDSTTTIANNLTSTVQVFTDDDYQLPYVVGNGTEGCLPAFPPQVFTLPQYGY ATLNRDNTENPTERSSFFCLEYFPSKMLRTGNNFEFTYNFEEVPFHSSFAPSQNLFKLAN PLVDQYLYRFVSTNNTGGVQFNKNLAGRYANTYKNWFPGPMGRTQGWNLGSGVNRA SVSAFATTNRMELEGASYQVPPQPNGMTNNLQGSNTYALENTMIFNSQPANPGTTATY LEGNMLITSESETQPVNRVAYNVGGQMATNNQSSTTAPATGTYNLQEIVPGSVWMERD VYLQGPIWAKIPETGAHFHPSPAMGGFGLKHPPPMMLIKNTPVPGNITSFSDVPVSSFITQ YSTGQVTVEMEWELKKENSKRWNPEIQYTNNYNDPQFVDFAPDSTGEYRTTRPIGTRY LTRPL) and to the amino acid residues of 389 to 397 region of VP3 (SEQ ID NO: 11; MSAGGGGPLGDNNQGADGVGNASGDWHCDSTWMGDRVVTKSTRTWVLPSYNNHQY REIKSGSVDGSNANAYFGYSTPWGYFDFNRFHSHWSPRDWQRLINNYWGFRPRSLRVK IFNIQVKEVTVQDSTTTIANNLTSTVQVFTDDDYQLPYVVGNGTEGCLPAFPPQVFTLPQ YGYATLNRDNTENPTERSSFFCLEYFPSKMLRTGNNFEFTYNFEEVPFHSSFAPSQNLFK LANPLVDQYLYRFVSTNNTGGVQFNKNLAGRYANTYKNWFPGPMGRTQGWNLGSGV NRASVSAFATTNRMELEGASYQVPPQPNGMTNNLQGSNTYALENTMIFNSQPANPGTT ATYLEGNMLITSESETQPVNRVAYNVGGQMATNNQSSTTAPATGTYNLQEIVPGSVWM ERDVYLQGPIWAKIPETGAHFHPSPAMGGFGLKHPPPMMLIKNTPVPGNITSFSDVPVSS FITQYSTGQVTVEMEWELKKENSKRWNPEIQYTNNYNDPQFVDFAPDSTGEYRTTRPIG TRYLTRPL). As used herein, “wild type 581 to 589 region” refers to a 581 to 589 region of VP1 having a sequence of ATGTYNLQE (SEQ ID NO: 9).

As used herein, “581 to 589 region” refers to a region or fragment of VP1 corresponding to amino acid residues 581 to 589 relative to the translational start of the VP1 polypeptide. The 581 to 589 region corresponds to amino acid residues 445 to 453 of VP2 and to amino acid residues 389 to 397 of VP3. The 581 to 589 region may confer tissue tropism or tissue preference to an AAV, and region variants may be engineered to confer tissue tropism or tissue preference to an rAAV formed from viral capsid polypeptides (VP1, VP2, and VP3) comprising the assembled variant. The VP1 with a generalized 581 to 589 region is provided in SEQ ID NO: 2 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPX¹X²X³X⁴X⁵X⁶X⁷X⁸X⁹IVPGSVWMERDVYLQGPIWAKIPETGAHFHPSPAMGGFGLK HPPPMMLIKNTPVPGNITSFSDVPVSSFITQYSTGQVTVEMEWELKKENSKRWNPEIQYT NNYNDPQFVDFAPDSTGEYRTTRPIGTRYLTRPL), in which the 581 to 589 region has a sequence of X¹X₂X³X⁴X₅X⁶X⁷X⁸X⁹ (SEQ ID NO: 5014); wherein X¹, X², X³, X⁴, X⁵, X⁶, X⁷, X₈, and X⁹ are each independently selected from A, R, N, D, C, E, Q, G, H, I, L, K, M, F, P, S, T, W, Y, and V.

It should be understood that the present disclosure includes polynucleotide sequences encoding for any sequence disclosed herein. For example, if an amino acid sequence is provided, the present disclosure also encompasses a polynucleotide sequence encoding for said amino acid sequence.

It should be understood that further embodiments include mutations in VP1, VP2, VP3, or any combination thereof that do not alter the desired properties (e.g., a particular tissue tropism) or affect viral assembly, as described herein.

An AAV virion is made of a capsid that may include the AAV5 VP capsid polypeptides disclosed herein (e.g., VP1, VP2, and VP3 capsid polypeptides).

### Engineered Capsids and Capsid Polypeptides for Tissue Tropism

Described herein are engineered capsids, engineered capsid polypeptides, and 581 to 589 regions of capsid polypeptides that confer tissue tropism or preference (e.g., tissue-specific accumulation, tissue-specific infection, or both) to a viral capsid. In some embodiments, an engineered capsid may comprise one or more engineered capsid polypeptides assembled into a recombinant adeno-associated virus (rAAV) viral capsid. In some embodiments, an engineered capsid polypeptide may comprise a 581 to 589 region. The 581 to 589 region of viral capsid polypeptides, corresponding to amino acid residues 581 to 589 of the VP1 polypeptide, amino acid residues 445 to 453 of the VP2 polypeptide, and amino acid residues 389 to 397 of the VP3 polypeptide, is located at an AAV interface that likely interacts with host cells and tissues.

In some embodiments, an engineered capsid polypeptide may comprise a 581 to 589 region that confers increased tropism or preference for retina tissue relative to other ocular tissues as compared to a wild type AAV capsid polypeptide (e.g., a wild type AAV5 capsid polypeptide of SEQ ID NO: 1). For example, a retina tissue-tropic capsid polypeptide may have increased tropism for retina tissue and may be de-targeted to photoreceptor tissue, as compared to a wild type AAV5 capsid polypeptide. In some embodiments, an engineered capsid polypeptide may comprise a 581 to 589 region that confers increased tropism or preference for retinal pigment epithelium (RPE) tissue relative to other ocular tissues as compared to a wild type AAV capsid polypeptide (e.g., a wild type AAV5 capsid polypeptide of SEQ ID NO: 1). For example, a RPE tissue-tropic capsid polypeptide may have increased tropism for RPE tissue and may be de-targeted to photoreceptor tissue, as compared to a wild type AAV5 capsid polypeptide. In some embodiments, an engineered capsid polypeptide may comprise a 581 to 589 region that confers increased tropism or preference for choroid tissue relative to other ocular tissues as compared to a wild type AAV capsid polypeptide (e.g., a wild type AAV5 capsid polypeptide of SEQ ID NO: 1). For example, a choroid tissue-tropic capsid polypeptide may have increased tropism for choroid tissue and may be de-targeted to photoreceptor tissue, as compared to a wild type AAV5 capsid polypeptide. In some embodiments, an engineered capsid polypeptide may comprise a 581 to 589 region that confers increased tropism or preference for RPE tissue and choroid tissue relative to other ocular tissues as compared to a wild type AAV capsid polypeptide (e.g., a wild type AAV5 capsid polypeptide of SEQ ID NO: 1).

Also described herein are methods of using engineered capsids comprising engineered capsid polypeptides with 581 to 589 regions for tissue-specific delivery of a payload (e.g., a polynucleotide, such as a transgene) encapsidated by the engineered capsid. Recombinant AAVs comprising VP capsid polypeptides with 581 to 589 regions engineered for tissue specificity may be used to specifically infect a target tissue. Using tissue-tropic rAAV viral capsids for payload delivery provides numerous advantages over using adeno-associated virus (AAV) viral capsids that lack tissue tropism including reduced toxicity, lower dose needed to produce a therapeutic effect, wider therapeutic window, and reduced immune response. Furthermore, tissue-specific payload delivery may improve tissue-specificity of therapies following local administration. For example, an ocular tissue-tropic rAAV viral capsid may be intravitreally administered to specifically deliver a payload to the eye for treatment of an ocular disease. Alternatively or in addition, an ocular tissue-tropic rAAV viral capsid may be administered by sub-retinal injection or topical mucosal administration (e.g., via eye drops) to deliver a payload to the eye for treatment of an ocular disease. In some embodiments, an ocular tissue-tropic rAAV viral capsid may be administered systemically.

Ocular tissue tropism of the rAAV may reduce systemic effects (e.g., liver toxicity) from the therapy that may otherwise occur despite local administration. For example, an ocular tissue-tropic rAAV viral capsid may be engineered to target a specific ocular tissue (e.g., retinal tissue) while detargeting other ocular tissues (e.g., choroid, vitreous humor, or aqueous humor). Ocular tissue-tropic rAAV viral capsids may target a specific ocular cell type, such as retinal pigment epithelial cells or photoreceptor cells (e.g., rod cells, cone cells, or both). In some embodiments, an ocular tissue-tropic rAAV viral capsids may target a specific ocular cell type (e.g., retinal pigment epithelial cells) while detargeting one or more other ocular tissue or cell types (e.g., photoreceptor cells, choroid, vitreous humor, or aqueous humor). Such ocular tissue-specific rAAVs may enable specific targeting of regions within the eye for treatment of tissue-specific conditions, such as conditions of the retina, optic nerve, or choroid. In yet another example, an ocular tissue-tropic rAAV may accumulate in specific ocular tissue (e.g., retina) following intravitreal administration, sub-retinal injection, topical mucosal administration, or systemic administration. An ocular tissue-tropic rAAV may target a specific ocular cell type (e.g., retinal pigment epithelial cells, photoreceptor cells, or both) following intravitreal administration, sub-retinal injection, topical mucosal administration, or systemic administration. For example, a retina tissue-tropic rAAV may specifically accumulate in retina tissue and may be de-targeted from photoreceptor tissue. In another example, a RPE tissue-tropic rAAV may specifically accumulate in RPE tissue and may be de-targeted from photoreceptor tissue. In another example, a choroid tissue-tropic rAAV may specifically accumulate in choroid tissue and may be de-targeted from photoreceptor tissue. In another example, a choroid and RPE tissue-tropic rAAV may specifically accumulate in choroid and RPE tissue and may be de-targeted from photoreceptor tissue.

In some embodiments, a tissue-tropic capsid of the present disclosure may be ocular tissue-tropic. For example, an ocular tissue-tropic capsid polypeptide may confer increased tropism or preference for one or more ocular tissues (e.g., retinal tissue) as compared to a wild type VP capsid polypeptide (e.g., SEQ ID NO: 1). An ocular tissue-tropic capsid polypeptide may confer cellular homing to one or more ocular cell types (e.g., retinal pigment epithelial cells, photoreceptor cells, rod photoreceptor cells, cone photoreceptor cells, or combinations thereof).

An engineered capsid polypeptide of the present disclosure may comprise one or more amino acid substitutions relative to an AAV5 viral protein (VP) polypeptide (e.g., a VP1 polypeptide of SEQ ID NO: 1, a VP2 polypeptide of SEQ ID NO: 10, or a VP3 polypeptide of SEQ ID NO: 11). The engineered capsid polypeptide may comprise one or more amino acid substitutions relative to a VP1 polypeptide of any or all of SEQ ID NO: 3-SEQ ID NO: 8. In some embodiments, the engineered capsid polypeptide may comprise a 581 to 589 region comprising one or more amino acid substitutions in a region of a VP polypeptide (e.g., a 581 to 589 region of VP1, VP2, VP3, or a combination thereof) corresponding to amino acid residues 581 to 589 of VP1 (e.g., SEQ ID NO: 1), amino acid residues 445 to 453 of VP2 (e.g., SEQ ID NO: 10), or amino acid residues 389 to 397 of VP3 (e.g., SEQ ID NO: 11). In some embodiments, the 581 to 589 region may be present in VP1, VP2, and VP3. An engineered viral capsid may be assembled from VP1, VP2, and VP3 polypeptides comprising a 581 to 589 region. In some embodiments, the 581 to 589 region may confer ocular tissue tropism or preference.

In some embodiments, an engineered capsid polypeptide of the present disclosure may comprise a 581 to 589 region that confers increased retina tissue-tropism or preference as compared to a wild type AAV capsid polypeptide (e.g., a wild type AAV5 capsid polypeptide of SEQ ID NO: 1). For example, a 581 to 589 region that confers increased retina tissue-tropism may have a sequenced of any one of SEQ ID NO: 14-SEQ ID NO: 2013 or SEQ ID NO: 2514-SEQ ID NO: 4513. In some embodiments, an engineered capsid polypeptide of the present disclosure may comprise a 581 to 589 region that confers increased RPE tissue-tropism or preference as compared to a wild type AAV capsid polypeptide (e.g., a wild type AAV5 capsid polypeptide of SEQ ID NO: 1). For example, a 581 to 589 region that confers increased RPE tissue-tropism may have a sequenced of any one of SEQ ID NO: 2014-SEQ ID NO: 2513 or SEQ ID NO: 4514-SEQ ID NO: 5013. In some embodiments, an engineered capsid polypeptide of the present disclosure may comprise a 581 to 589 region that confers increased choroid tissue-tropism or preference as compared to a wild type AAV capsid polypeptide (e.g., a wild type AAV5 capsid polypeptide of SEQ ID NO: 1). For example, a 581 to 589 region that confers increased choroid tissue-tropism may have a sequenced of any one of SEQ ID NO: 2014-SEQ ID NO: 2513 or SEQ ID NO: 4514-SEQ ID NO: 5013.

### Capsid Engineering Methods

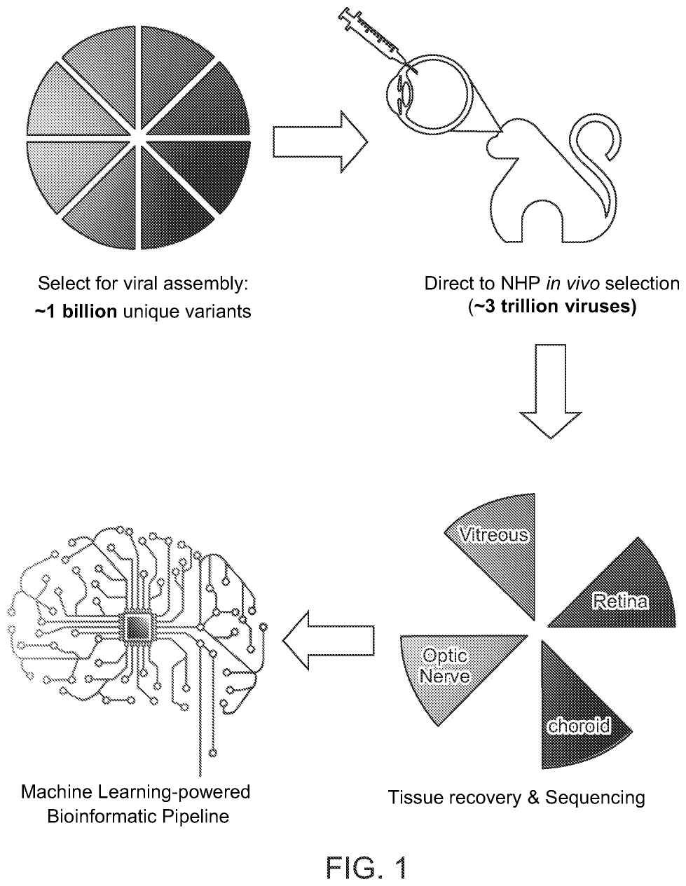

Disclosed herein is a system for high throughput engineering of engineered AAV capsids with modified function, including increased or decreased infectivity of desired tissues, such as increased targeting of ocular tissue (e.g., retinal tissue), decreased targeting of non-eye tissue, or selective targeting of a specific ocular tissue (e.g., retinal tissue) compared to other ocular tissues (e.g., aqueous humor or vitreous humor), relative to a wild type AAV5 capsid (e.g., comprising a VP capsid polypeptide of SEQ ID NO: 1). In some embodiments, the engineered AAV capsids may exhibit cellular homing to specific ocular cell types (e.g., retinal pigment epithelial cells, photoreceptor cells, rod photoreceptor cells, cone photoreceptor cells, or combinations thereof). A general schematic of the process is shown in FIG. 1. However, it should be understood that the present disclosure also encompasses reasonable variations or extensions to the method that are understood to those of ordinary skill in the art. The method may begin with production of a capsid library with theoretical diversity of 5×10¹¹ unique sequence variants. Higher or lower theoretical diversities are also encompassed herein. For example, a capsid library may have a theoretical diversity of from about 1×10³ to about 1×10²⁰. As shown in FIG. 1, the library may first be selected for the ability to assemble into viral capsids. The library may then be cloned into plasmids and transformed into bacteria. As shown in FIG. 1, the library plasmids may be subsequently screened for productive virion assembly in a production cell line. The assembled virions may then be administered intravitreally into non-human primates (NHP). In some embodiments, the assembled virions may be administered via sub-retinal injection, topical mucosal administration, or systemic administration. After a period of time sufficient for distribution, infection, and stable transduction, the NHP may be sacrificed, organs harvested, and sequences of AAV capsids in each tissue may be determined by deep sequencing. Eye tissues, including aqueous humor, choroid, retina, optic nerve, vitreous humor, and remaining eye tissue may be harvested for sequencing.

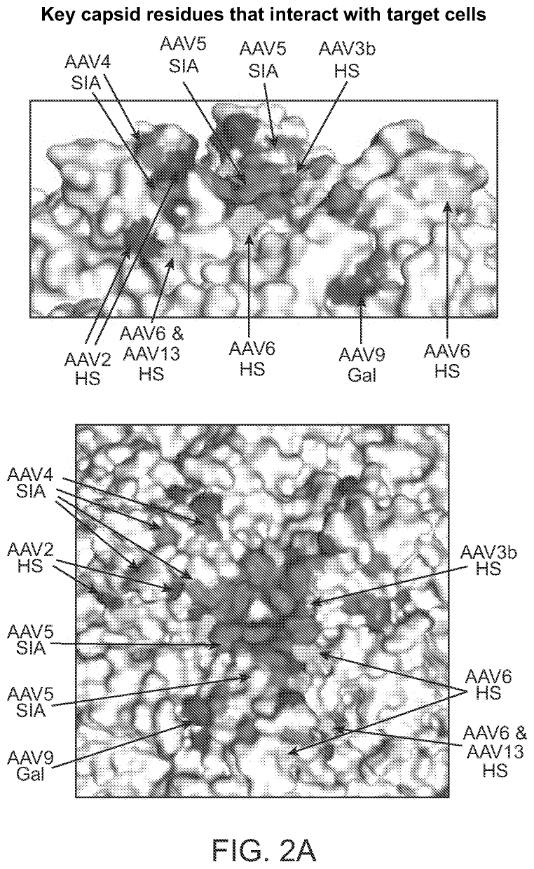

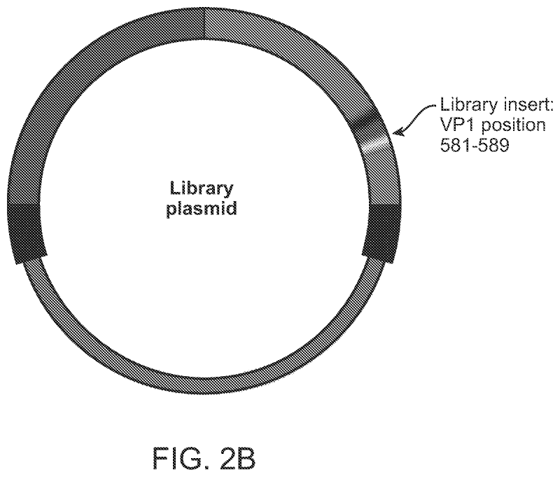

FIG. 2A provides a side view (top panel) and top view (bottom panel) of the surface of a prototype AAV virion, identifying residues of known AAV capsids, including AAV2, AAV5, AAV6, and AAV9, that have been shown in the research literature to interact with target cells. These target-interacting residues correspond to amino acids 581 to 589 in the AAV5 VP1 capsid protein.

FIG. 2B shows the salient elements of the library plasmid, illustrating rep and cap coding sequences positioned between AAV ITRs. In the illustrated embodiment, further described in EXAMPLE 1, variation is introduced into each of residues 581 to 589 of the AAV5 cap protein (“Library 581 to 589 region”) that is present in assembled VP1, VP2, and VP3 polypeptides. Each of the 20 natural amino acids is introduced at each of the 9 positions of the 581 to 589 region, providing a theoretical library diversity of 20⁹ (20\hat{ }9; approximately 5×10¹¹) unique sequence variants.

The 581 to 589 region targeted for engineering is the most likely to interact with target cell receptors, and relatively tolerant to changes without disrupting virion assembly. Unlike earlier approaches that add unstructured peptides that protrude above the virion 3-fold axis of symmetry, the current approach introduces sequence diversity that alters the characteristics of the binding pocket. In addition, this approach may change the overall structure of the receptor-binding trimer, allowing for altered allosteric interactions outside the binding pocket (e.g., AAVR PKD1). Introduced diversity is non-random, thereby reducing missense and frameshifts of randomized libraries.

By cloning the polynucleotide encoding the capsid variants into the packaged viral genome (between the ITRs), the recombinant virions with variant capsids carry polynucleotides having their cognate mutation, so the unique variant providing the desired function can be identified by sequencing packaged virus or infected cells.

In some embodiments, the capsid is a capsid selected from AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, or AAVhu68, (described in WO2020/033842, incorporated herein by reference in its entirety). For example, the capsid may be an AAV5 capsid. The hu68 capsid is described in WO 2018/160582, incorporated herein by reference in its entirety.

Such capsids may comprise a 581 to 589 region corresponding amino acid residues 581 to 589 of the AAV5 VP1, and as such analogous engineered VP capsids with desired tissue tropism, ability to assemble, and exhibit various other desired traits are encompassed herein. Thus, any one of the engineered AAV5 VP capsid polypeptides disclosed herein having a mutation or substitution in the 581 to 589 region corresponding to the 581 to 589 region of AAV5 VP1 may be inserted into the corresponding region in any one of the other AAV capsids described herein and the present disclosure encompasses such variants.

In some embodiments, the capsid is a derivative, modification, or pseudotype of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, or AAVhu68. For example, the capsid may be a derivative of AAV5.

In some embodiments, capsid protein is a chimera of capsid proteins from two or more serotype selected from AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC17, AAVhu68, and AAV.HSC16 (described in WO2020/033842, incorporated herein by reference in its entirety). In certain embodiments, the capsid is an rh32.33 capsid, described in U.S. Pat. No. 8,999,678, incorporated herein by reference in its entirety.

Such capsids may comprise a 581 to 589 region corresponding to 581 to 589 of the AAV5 VP1, and as such analogous engineered VP capsids with desired tissue tropism, ability to assemble, and exhibit various other desired traits are encompassed herein.

### VP-Encoding Polynucleotides, Vectors, and Vector Libraries

Accordingly, in a first aspect, polynucleotides are provided. The polynucleotides encode an adeno-associated virus (AAV) VP1 capsid polypeptide having the amino acid sequence of SEQ ID NO: 2, wherein X¹, X², X³, X⁴, X⁵, X⁶, X⁷, X⁸, and X⁹ are each independently selected from the 20 naturally occurring amino acids, using standard one letter codes, from A, R, N, D, C, E, Q, G, H, I, L, K, M, F, P, S, T, W, Y, and V. The sequence X¹X₂X³X⁴X⁵X⁶X⁷X⁸X⁹ of SEQ ID NO: 2 corresponds to the 581 to 589 region of VP1. X¹ corresponds to a position 581 of the 581 to 589 region. X² corresponds to a position 582 of the 581 to 589 region. X³ corresponds to a position 583 of the 581 to 589 region. X⁴ corresponds to a position 584 of the 581 to 589 region. X⁵ corresponds to a position 585 of the 581 to 589 region. X⁶ corresponds to a position 586 of the 581 to 589 region. X⁷ corresponds to a position 587 of the 581 to 589 region. X⁸ corresponds to a position 588 of the 581 to 589 region. X⁹ corresponds to a position 589 of the 581 to 589 region. The polynucleotide encodes a polypeptide that includes at least one mutation of the native AAV5 capsid and thus does not have the sequence of SEQ ID NO: 1. In addition, the polypeptide does not have the sequence of SEQ ID NO: 3, SEQ ID NO: 4, SEQ ID NO: 5, SEQ ID NO: 6, SEQ ID NO: 7, or SEQ ID NO: 8. In some embodiments, the VP1 capsid polypeptide comprises a 581 to 589 region. For example, a VP1 capsid polypeptide comprising a 581 to 589 region may comprise a sequence of SEQ ID NO: 1 in which residues 581 to residue 589 are replaced with a sequence of X¹X²X³X⁴X⁵X⁶X⁷X⁸X⁹ (SEQ ID NO: 5014), wherein X¹, X², X³, X⁴, X⁵, X⁶, X⁷, X⁸, and X⁹ are each independently selected from A, R, N, D, C, E, Q, G, H, I, L, K, M, F, P, S, T, W, Y, and V, and wherein the VP1 capsid polypeptide does not have the sequence of any of SEQ ID NO: 1, SEQ ID NO: 3 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPATGTVNLQEIVPGSVWMERDVYLQGPIWAKIPETGAHFHPSPAMGGFGLKHPPPM MLIKNTPVPGNITSFSDVPVSSFITQYSTGQVTVEMEWELKKENSKRWNPEIQYTNNYN DPQFVDFAPDSTGEYRTTRPIGTRYLTRPL), SEQ ID NO: 4 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPATGTYNTQEIVPGSVWMERDVYLQGPIWAKIPETGAHFHPSPAMGGFGLKHPPPM MLIKNTPVPGNITSFSDVPVSSFITQYSTGQVTVEMEWELKKENSKRWNPEIQYTNNYN DPQFVDFAPDSTGEYRTTRPIGTRYLTRPL), SEQ ID NO: 5 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPTTGTYNLQEIVPGSVWMERDVYLQGPIWAKIPETGAHFHPSPAMGGFGLKHPPPM MLIKNTPVPGNITSFSDVPVSSFITQYSTGQVTVEMEWELKKENSKRWNPEIQYTNNYN DPQFVDFAPDSTGEYRTTRPIGTRYLTRPL), SEQ ID NO: 6 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPAAGTYNLQEIVPGSVWMERDVYLQGPIWAKIPETGAHFHPSPAMGGFGLKHPPPM MLIKNTPVPGNITSFSDVPVSSFITQYSTGQVTVEMEWELKKENSKRWNPEIQYTNNYN DPQFVDFAPDSTGEYRTTRPIGTRYLTRPL), SEQ ID NO: 7 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPATGAYNLQEIVPGSVWMERDVYLQGPIWAKIPETGAHFHPSPAMGGFGLKHPPPM MLIKNTPVPGNITSFSDVPVSSFITQYSTGQVTVEMEWELKKENSKRWNPEIQYTNNYN DPQFVDFAPDSTGEYRTTRPIGTRYLTRPL), or SEQ ID NO: 8 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPATGTVNTQEIVPGSVWMERDVYLQGPIWAKIPETGAHFHPSPAMGGFGLKHPPPM MLIKNTPVPGNITSFSDVPVSSFITQYSTGQVTVEMEWELKKENSKRWNPEIQYTNNYN DPQFVDFAPDSTGEYRTTRPIGTRYLTRPL).

In some embodiments, a polynucleotide of the present disclosure may encode an AAV VP1 capsid polypeptide having the amino acid sequence of SEQ ID NO: 2, wherein X¹X²X³X⁴X⁵X⁶X⁷X⁸X⁹ of SEQ ID NO: 2 is any one of SEQ ID NO: 14-SEQ ID NO: 2013 or SEQ ID NO: 2514-SEQ ID NO: 4513. In some embodiments, a polynucleotide of the present disclosure may encode an AAV VP1 capsid polypeptide having the amino acid sequence of SEQ ID NO: 2, wherein X¹X²X³X⁴X⁵X⁶X⁷X⁸X⁹ of SEQ ID NO: 2 is any one of SEQ ID NO: 2014-SEQ ID NO: 2513 or SEQ ID NO: 4514-SEQ ID NO: 5013.

In some embodiments, the polynucleotide encodes an AAV VP1 capsid polypeptide that further comprises one or more mutations at an amino acid residue outside of the 581 to 589 region, with reference to SEQ ID NO: 1, wherein the resulting recombinant capsid is capable of forming an assembled virion that exhibits desired tissue targeting as compared to wild type AAV5 (SEQ ID NO: 1). The one or more mutations may confer increased tissue tropism or preference (e.g., increased retinal tissue tropism) on the assembled virion as compared to a wild type AAV capsid polypeptide (e.g., a wild type AAV5 capsid polypeptide of SEQ ID NO: 1).

In another aspect, a vector capable of replication in prokaryotic cells is provided, wherein the vector comprises the polynucleotide described immediately above. In typical embodiments, the vector is a plasmid encoding a replication competent AAV genome.

In a further aspect, a library is provided. The library comprises a plurality of vectors comprising the AAV capsid-encoding polynucleotides. In some embodiments, the vectors are plasmids, and the plurality of plasmids comprise a plurality of different AAV VP-encoding polynucleotides. The library may comprise a plurality of vectors encoding AAV VP1 capsid polypeptides having the amino acid sequence of SEQ ID NO: 2 comprising one or more amino acid variations in the 581 to 589 region.

In various embodiments, the library encodes at least 1×10⁹ different AAV VP capsid polypeptides, at least 2.5×10⁹ different AAV VP capsid polypeptides, at least 5×10⁹ different AAV VP capsid polypeptides, at least 7.5×10⁹ different AAV VP capsid polypeptides, at least 1×10¹⁰ different AAV VP capsid polypeptides, at least 2.5×10¹⁰ different AAV VP capsid polypeptides, at least 5×10¹⁰ different AAV VP capsid polypeptides, at least 7.5×10¹⁰ different AAV VP capsid polypeptides, at least 1×10¹¹ different AAV VP capsid polypeptides, at least 2.5×10¹¹ different AAV VP capsid polypeptides, or at least 5×10¹¹ different AAV VP capsid polypeptides. The library may encode VP capsid polypeptides comprising 581 to 589 region variants.

In another aspect, prokaryotic cells comprising the vectors are provided. In some embodiments, the prokaryotic cell is an E. coli cell and the vector is a plasmid.

In a related aspect, libraries are provided, the library comprising a plurality of E. coli cells, wherein the plurality of cells comprise a plurality of plasmids, wherein the plurality of plasmids comprise a plurality of different AAV VP-encoding polynucleotides.

In some embodiments, the library encodes at least 1×10⁹ different AAV VP capsid polypeptides, at least 2.5×10⁹ different AAV VP capsid polypeptides, at least 5×10⁹ different AAV VP capsid polypeptides, at least 7.5×10⁹ different AAV VP capsid polypeptides, at least 1×10¹⁰ different AAV VP capsid polypeptides, at least 5×10¹⁰ different AAV VP capsid polypeptides, at least 7.5×10¹⁰ different AAV VP capsid polypeptides, at least 1×10¹¹ different AAV VP capsid polypeptides, at least 2.5×10¹¹ different AAV VP capsid polypeptides, or at least 5×10¹¹ different AAV VP capsid polypeptides. The library may encode VP capsid polypeptides comprising 581 to 589 region variants.

### VP Polypeptides, Peptide Libraries

In another aspect, AAV VP1 capsid polypeptides are provided. The polypeptide has the amino acid sequence of SEQ ID NO: 2, wherein X¹, X², X³, X⁴, X⁵, X⁶, X⁷, X⁸, and X⁹ are each independently selected from A, R, N, D, C, E, Q, G, H, I, L, K, M, F, P, S, T, W, Y, and V. The polypeptide includes at least one mutation as compared to native AAV VP1, and thus does not have the sequence of SEQ ID NO: 1. In addition, the polypeptide does not have the sequence of SEQ ID NO: 3, SEQ ID NO: 4, SEQ ID NO: 5, SEQ ID NO: 6, SEQ ID NO: 7, or SEQ ID NO: 8.

In some embodiments, the polypeptide further comprises one or more mutations at an amino acid residue outside of the 581 to 589 region, with reference to SEQ ID NO: 1, wherein the resulting recombinant capsid is capable of forming an assembled virion that exhibits desired tissue targeting as compared to a wild type AAV5 comprising a VP capsid polypeptide of SEQ ID NO: 1.

In a further aspect, libraries are provided, the libraries comprising a plurality of polypeptides as described immediately above, the plurality having different primary amino acid sequences. A library may comprise a plurality of polypeptides of SEQ ID NO: 2 comprising one or more amino acid variations in the 581 to 589 region.

In some embodiments, library comprises at least 1×10⁹ different AAV VP capsid polypeptides, at least 2.5×10⁹ different AAV VP capsid polypeptides, at least 5×10⁹ different AAV VP capsid polypeptides, at least 7.5×10⁹ different AAV VP capsid polypeptides, at least 1×10¹⁰ different AAV VP capsid polypeptides, at least 2.5×10¹⁰ different AAV VP capsid polypeptides, at least 5×10¹⁰ different AAV VP capsid polypeptides, at least 7.5×10¹⁰ different AAV VP capsid polypeptides, at least 1×10¹¹ different AAV VP capsid polypeptides, at least 2.5×10¹¹ different AAV VP capsid polypeptides, or at least 5×10¹¹ different AAV VP capsid polypeptides. The library may comprise VP capsid polypeptides comprising 581 to 589 region variants.

In certain embodiments, the library comprises at least from about 1×10⁵ to at least about 5×10¹¹ different AAV VP capsid polypeptides. In certain embodiments, the library comprises at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, at least about 1×10⁷, at least about 2×10⁷, at least about 3×10⁷, at least about 4×10⁷, at least about 5×10⁷, at least about 6×10⁷, at least about 7×10⁷, at least about 8×10⁷, at least about 9×10⁷, at least about 1×10⁸, at least about 2×10⁸, at least about 3×10⁸, at least about 4×10⁸, at least about 5×10⁸, at least about 6×10⁸, at least about 7×10⁸, at least about 8×10⁸, at least about 9×10⁸, at least about 1×10⁹, at least about 2×10⁹, at least about 3×10⁹, at least about 4×10⁹, at least about 5×10⁹, at least about 6×10⁹, at least about 7×10⁹, at least about 8×10⁹, at least about 9×10⁹, at least about 1×10¹⁰, at least about 2×10¹⁰, at least about 3×10¹⁰, at least about 4×10¹⁰, at least about 5×10¹⁰, at least about 6×10¹⁰, at least about 7×10¹⁰, at least about 8×10¹⁰, at least about 9×10¹⁰, at least about 1×10¹¹, at least about 2×10¹¹, at least about 3×10¹¹, at least about 4×10¹¹, or at least about 5×10¹¹ AAV VP capsid polypeptides.

In certain embodiments, provided herein is a recombinant adeno-associated virus AAV VP1 capsid polypeptide having at least one mutation in a residue of region 581 to residue 589 in SEQ ID NO: 1, inclusive, wherein the mutation confers at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.2-fold, at least about 2.4-fold, at least about 2.6-fold, at least about 2.8-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 100-fold, at least about 200-fold, at least about 300-fold, at least about 400-fold, or at least about 500-fold increased accumulation of an AAV virion having said AAV VP1 capsid polypeptide in a target tissue (e.g., a retinal tissue), target cell (e.g., retinal pigment epithelial cells, photoreceptor cells, rod photoreceptor cells, cone photoreceptor cells, or combinations thereof), or both as compared to a non-target tissue (e.g., vitreous humor or aqueous humor), as compared to wild type AAV virion having a wild type AAV5 VP1 capsid polypeptide, and wherein the AAVVP1 capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. In some embodiments, a VP1 capsid polypeptide comprises a 581 to 589 region comprising one or more amino acid mutations. For example, a VP1 capsid polypeptide may comprise a sequence of SEQ ID NO: 1 in which residues 581 to residue 589 are replaced with a sequence of X¹X²X³X⁴X⁵X⁶X⁷X⁸X⁹ (SEQ ID NO: 5014), wherein X¹, X², X³, X⁴, X⁵, X⁶, X⁷, X⁸, and X⁹ are each independently selected from A, R, N, D, C, E, Q, G, H, I, L, K, M, F, P, S, T, W, Y, and V, and wherein the VP1 capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8.

In some embodiments, an AAV VP1 capsid polypeptide of the present disclosure may have an amino acid sequence of SEQ ID NO: 2, wherein X¹X²X³X⁴X⁵X⁶X⁷X⁸X⁹ of SEQ ID NO: 2 is any one of SEQ ID NO: 14-SEQ ID NO: 2013 or SEQ ID NO: 2514-SEQ ID NO: 4513. In some embodiments, an AAV VP1 capsid polypeptide of the present disclosure may have an amino acid sequence of SEQ ID NO: 2, wherein X¹X²X³X⁴X⁵X⁶X⁷X⁸X⁹ of SEQ ID NO: 2 is any one of SEQ ID NO: 2014-SEQ ID NO: 2513 or SEQ ID NO: 4514-SEQ ID NO: 5013.

rAAV Virions, Virion Libraries

In another aspect, recombinant AAV virions (rAAV) are provided. The virion comprises an AAV VP capsid polypeptide as described above. In some embodiments, the rAAV has increased tropism for primate and human ocular tissue (e.g., retinal tissue), or may have increased accumulation in ocular cells (e.g., retinal pigment epithelial cells, photoreceptor cells, rod photoreceptor cells, cone photoreceptor cells, or combinations thereof), or both, as compared to a rAAV having the native AAV5 VP1 capsid polypeptide (SEQ ID NO:1). In some embodiments, the rAAV has increased ability to assemble, or exhibits greater virion stability, as compared to a rAAV having the native AAV5 VP1 capsid polypeptide (SEQ ID NO: 1).

In certain of these embodiments, the rAAV has increased ability to infect one or more ocular tissues selected from aqueous humor, choroid, retina, optic nerve, vitreous humor, and combinations thereof following intravitreal administration, sub-retinal injection, topical mucosal administration, or systemic administration as compared to a rAAV having the native AAV5 VP1 capsid polypeptide (SEQ ID NO: 1). In some embodiments, the rAAV has increased tissue tropism for retinal tissue compared to a wild type AAV5 capsid. In some embodiments, the rAAV has increased accumulation in photoreceptor cells (e.g., rod photoreceptor cells, cone photoreceptor cells, or a combination thereof), retinal pigment epithelial cells, or both, compared to a wild type AAV5 capsid.

Additionally, provided are polynucleotide sequences encoding the rAAV capsid VP proteins described herein.

In a further aspect, libraries are provided that comprise a plurality of rAAV as described above. The plurality of rAAVs comprise a plurality of VP capsid polypeptides having different primary amino acid sequences. An rAAV of the plurality of rAAVs may comprise a polypeptide of SEQ ID NO: 2 comprising one or more amino acid variations in the 581 to 589 region.

In some embodiments, the library comprises at least 1×10⁹ different AAV VP capsid polypeptides, at least 2.5×10⁹ different AAV VP capsid polypeptides, at least 5×10⁹ different AAV VP capsid polypeptides, at least 7.5×10⁹ different AAV VP capsid polypeptides, at least 1×10¹⁰ different AAV VP capsid polypeptides, at least 2.5×10¹⁰ different AAV VP capsid polypeptides, at least 5×10¹⁰ different AAV VP capsid polypeptides, at least 7.5×10¹⁰ different AAV VP capsid polypeptides, at least 1×10¹¹ different AAV VP capsid polypeptides, at least 2.5×10¹¹ different AAV VP capsid polypeptides, or at least 5×10¹¹ different AAV VP capsid polypeptides. The library may comprise AAV VP capsid polypeptides comprising 581 to 589 region variants. For example, a VP1 capsid polypeptide may comprise a sequence of SEQ ID NO: 1 in which residues 581 to residue 589 are replaced with a sequence of X¹X²X³X⁴X⁵X⁶X⁷X⁸X⁹ (SEQ ID NO: 5014), wherein X¹, X², X³, X⁴, X⁵, X⁶, X⁷, X⁸, and X⁹ are each independently selected from A, R, N, D, C, E, Q, G, H, I, L, K, M, F, P, S, T, W, Y, and V, and wherein the VP1 capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8.

In some embodiments, an AAV virion of the present disclosure may comprise an AAV VP1 capsid polypeptide having an amino acid sequence of SEQ ID NO: 2, wherein X¹X²X³X⁴X⁵X⁶X⁷X⁸X⁹ of SEQ ID NO: 2 is any one of SEQ ID NO: 14-SEQ ID NO: 2013 or SEQ ID NO: 2514-SEQ ID NO: 4513. In some embodiments, an AAV virion of the present disclosure may comprise an AAV VP1 capsid polypeptide having an amino acid sequence of SEQ ID NO: 2, wherein X¹X²X³X⁴X⁵X⁶X⁷X⁸X⁹ of SEQ ID NO: 2 is any one of SEQ ID NO: 2014-SEQ ID NO: 2513 or SEQ ID NO: 4514-SEQ ID NO: 5013.

### Pharmaceutical Compositions

In another aspect, pharmaceutical compositions are provided. The pharmaceutical composition comprises a rAAV as described above and a pharmaceutically acceptable carrier.

A pharmaceutical composition can comprise a first active ingredient. The first active ingredient can comprise a viral vector as described herein and/or any payload as described herein. The pharmaceutical composition can be formulated in unit dose form. The pharmaceutical composition can comprise a pharmaceutically acceptable excipient, diluent, or carrier. The pharmaceutical composition can comprise a second, third, or fourth active ingredient—such as to facilitate enhanced gene replacement, RNA editing, DNA editing, or imaging.

A pharmaceutical composition described herein can compromise an excipient. An excipient can comprise a cryo-preservative, such as DMSO, glycerol, polyvinylpyrrolidone (PVP), or any combination thereof. An excipient can comprise a cryo-preservative, such as a sucrose, a trehalose, a starch, a salt of any of these, a derivative of any of these, or any combination thereof. An excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a triglyceride, an alcohol, or any combination thereof. An excipient can comprise sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerol, xanthan rubber, soy isoflavones, polysorbate 80, ethyl alcohol, water, teprenone, or any combination thereof.

Compositions and methods provided herein can utilize pharmaceutical compositions. The compositions described throughout can be formulated into a pharmaceutical and be used to treat a human or mammal, in need thereof, diagnosed with a disease. In some cases, pharmaceutical compositions can be used prophylactically.

The compositions provided herein can be utilized in methods provided herein. Any of the provided compositions provided herein can be utilized in methods provided herein. In some cases, a method comprises at least partially preventing, reducing, ameliorating, and/or treating a disease or condition, or a symptom of a disease or condition. A subject can be a human or non-human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician).

### Methods of Treatment or Detection

In some aspects, the present disclosure provides for methods of treatment using an rAAV virion having any one of the engineered AAV VP capsid polypeptide sequences disclosed herein. In some aspects, the present disclosure provides for methods of detection using an rAAV virion having any one of the engineered AAV VP capsid polypeptide sequences disclosed herein. A method of treatment may comprise administering to a subject an effective amount of a pharmaceutical composition comprising rAAV virions assembled from VP polypeptides comprising a 581 to 589 region that confers tissue tropism or preference (e.g., ocular tissue tropism) to the rAAV. The rAAV virions may encapsidate any payload, including those payloads disclosed herein. For example, a method of treatment may comprise administering an effective amount of a pharmaceutical composition comprising rAAV virions assembled from VP polypeptides comprising a 581 to 589 region having a sequence of any one of SEQ ID NO: 14-SEQ ID NO: 2013 or SEQ ID NO: 2514-SEQ ID NO: 4513. In another example, a method of treatment may comprise administering an effective amount of a pharmaceutical composition comprising rAAV virions assembled from VP polypeptides comprising a 581 to 589 region having a sequence of any one of SEQ ID NO: 2014-SEQ ID NO: 2513 or SEQ ID NO: 4514-SEQ ID NO: 5013.

In some embodiments, the effective amount is at least 1×10⁸ viral genomes per dose. In some embodiments, the effective amount is at least 5×10⁸ viral genomes/dose, 7.5×10⁸ viral genomes/dose, at least 1×10⁹ viral genomes/dose, at least 2.5×10⁹ viral genomes/dose, at least 5×10⁹ viral genomes/dose. In some embodiments, an effective amount of an rAAV assembled from VP polypeptides comprising a 581 to 589 region conferring ocular tissue tropism or preference (e.g., retinal tissue tropism) may be lower than an effective amount of an AAV assembled from VP1, VP2, and VP3 polypeptides of SEQ ID NO: 1, SEQ ID NO: 10, and SEQ ID NO: 11, respectively.

In some embodiments, the effective amount is at least 1×10¹¹ viral genomes, at least 5×10¹¹ viral genomes, at least 1×10¹² viral genomes, at least 5×10¹² viral genomes, at least 1×10¹³ viral genomes, at least 1×10¹⁴ viral genomes, or at least 5×10¹⁴ viral genomes. In some embodiments, an effective amount of an rAAV assembled from VP polypeptides comprising a 581 to 589 region conferring ocular tissue tropism may be lower than an effective amount of an AAV assembled from VP1, VP2, and VP3 polypeptides of SEQ ID NO: 1, SEQ ID NO: 10, and SEQ ID NO: 11, respectively.

In some embodiments, the rAAV virion is administered via an ocular administration route, such as intravitreal administration, subconjunctival administration, retrobulbar administration, sub-retinal injection, topical mucosal administration, or intracameral administration. In some embodiments, the rAAV virion is administered via a systemic administration route including enteral routes of administration and parenteral routes of administration. The rAAV virion may be administered intravenously. In some embodiments, the rAAV may be administered intramuscularly. In some embodiments, the rAAV may be administered intraperitoneally. In some embodiments, the rAAV may be administered topically. In some embodiments, the rAAV may be administered orally. In particular embodiments, the rAAV virion is administered intravenously. In some embodiments, the rAAV is administered intrathecally. In some embodiments, the rAAV is administered by intracerebral ventricular injection. In some embodiments, the rAAV is administered by intracisternal magna administration. In some embodiments, the rAAV is administered by intravitreal injection.

In various embodiments, the patient suffers from one of the conditions listed in TABLE 1, below. In particular embodiments, the patient suffers from one of the conditions listed in TABLE 1 and the rAAV includes a payload comprising the transgene product associated therewith in TABLE 1. In some embodiments, an rAAV may be selected to specifically target the primary gene delivery target (e.g., the eye). For example, an rAAV selected to target the eye may comprise VP capsid polypeptides comprising a 581 to 589 region that confers ocular tissue tropism.

In some embodiments, an rAAV virion of the present disclosure, having any of the engineered AAV VP capsid polypeptide sequences disclosed herein, comprises a vector genome, the vector genome comprising a therapeutic polynucleotide or payload. In further embodiments, said payload may be under control of regulatory sequences that direct expression in infected human cells. In some embodiments, the payload may be under control of a cell type- or tissue type-specific promoter to direct expression in a target cell type or target tissue type. For example, expression of the payload may be under control of a retinal-specific promoter to promote retinal-specific expression, a retinal pigment epithelium-specific promoter to promote retinal pigment epithelium-specific expression, a choroid-specific promoter to promote choroid-specific expression, a photoreceptor-specific promoter to promote photoreceptor-specific expression, or an optic nerve-specific promoter to promote optic nerve-specific expression. In some embodiments, the payload comprises a therapeutic polynucleotide encoding any genetically encodable payload, such as an RNA (e.g., a guide RNA), a suppressor tRNA, a transgene, or a genome modifying entity.

In some embodiments, the therapeutic polynucleotide encodes a guide RNA, a tRNA, a suppressor tRNA, a siRNA, a miRNA, an mRNA, a shRNA, a circular RNA, or an antisense oligonucleotide (ASO), a ribozyme, a DNAzyme, an aptamer, or any combination thereof. In some embodiments, the therapeutic polynucleotide encodes a linear therapeutic polynucleotide or a circular therapeutic polynucleotide.

In some embodiments, the therapeutic polynucleotide is a transgene, encoding a therapeutic protein. In some embodiments, the therapeutic protein is an antibody that binds a target molecule (e.g., an anti-VEGF antibody that binds VEGF). In particular embodiments, the transgene encodes a protein selected from the targets suitable for modification or transgene products of TABLE 1. In some embodiments, the transgene encodes a protein that binds a target provided in TABLE 1. In some embodiments, the payload encodes a polynucleotide that targets a target provided in TABLE 1. In some embodiments, an ocular payload or a transgene encoding an ocular target is encapsidated by an rAAV comprising VP capsid polypeptides comprising a 581 to 589 region that confers ocular tissue tropism or preference.

TABLE 1
Suitable Therapeutic Targets or Payloads

| Condition | Payload or Target |
|---|---|
| 3-Methylglutaconic Aciduria, Type Iii | AFG3L2, MECR, OPA1 |
| Abetalipoproteinemia | MTTP |
| Abnormal Threshold of Rods | HBA2, PROC |
| Aceruloplasminemia | CP |
| Achromatopsia | ATF6, CABP4, CNGA3, CNGB3, PDE6C |
| Achromatopsia 3 | CNGB3 |
| Achromatopsia 4 | GNAT2 |
| ad Retinitis Pigmentosa | NRL, RDH12, PRPH2 (RDS), RHO, RPGR, |
| | SNRNP200, NR2E3, IMPDH1, CRX, HK1, |
| | IMPDH2, SNRNP200, PRPF3, AGBL5 |
| Age-related macular degeneration | NOS2A, CFH, CF, C2, C3, C5A, CFB, |
| (AMD), e.g., wet AMD, or dry AMD | HTRA1/LOC, MMP-9, TIMP-3, SLC16A8, |
| | VEGFA, VEGFC, CD59, RHO |
| Aland Island Eye Disease | CACNA1F |
| Albinism | TYRP1 |
| Albinism, Ocular, Type I | GPR143 |
| Albinism, Oculocutaneous, Type Ia | TYR |
| Albinism, Oculocutaneous, Type Ii | MC1R, OCA2 |
| Alport Syndrome | COL4A3, COL4A4, COL4A5 |
| Alzheimer Disease, Familial, 1 | APP, GRN, PSEN1 |
| Amblyopia | CACNA1F |
| Amyotrophic Lateral Sclerosis 2, | ALS2 |
| Juvenile | |
| Anterior Segment Dysgenesis 2 | FOXE3 |
| Auditory Neuropathy and Optic | FDXR |
| Atrophy | |
| Autosomal Dominant Cerebellar | KAT6B |
| Ataxia | |
| Autosomal Dominant Congenital | SNRNP200 |
| Stationary Night Blindness | |
| Bardet-Biedl Syndrome | ARL6, BBS1, BBS10, BBS12, BBS2, BBS4, BBS5, |
| | BBS7, BBS9, CEP290, IFT172, MKKS, MKS1, |
| | SDCCAG8, TRIM32, TTC8, WDPCP |
| Bardet-Biedl Syndrome 1 | BBIP1, BBS1, BBS10, LZTFL1 |
| Bardet-Biedl Syndrome 13 | MKS1 |
| Bardet-Biedl Syndrome 17 | LZTFL1 |
| Bardet-Biedl Syndrome 2 | BBS2, MKKS |
| Bardet-Biedl Syndrome 3 | ARL6 |
| Bardet-Biedl Syndrome 5 | BBS5 |
| Bardet-Biedl Syndrome 8 | TTC8 |
| Bardet-Biedl Syndrome 9 | BBS9 |
| Bartter Syndrome, Type 3 | CLCNKB |
| Basal Laminar Drusen | CFH |
| Bestrophinopathy, Autosomal | BEST1 |
| Recessive | |
| Bietti Crystalline Corneoretinal | CYP4V2 |
| Dystrophy | |
| Blau Syndrome | NOD2 |
| Blue Cone Monochromacy | OPN1LW |
| Bohring-Opitz Syndrome | ASXL1 |
| Bosma Arhinia Microphthalmia | SMCHD1 |
| Syndrome | |
| Boucher-Neuhauser Syndrome | PNPLA6 |
| Branchiooculofacial Syndrome | EYA1, TFAP2A |
| Butterfly-Shaped Pigment Dystrophy | CTNNA1, PRPH2 |
| Cafe-Au-Lait Spots, Multiple | NF1 |
| Cataracts | GEMIN4, CYP51A1, RIC1, TAPT1, TAF1A, |
| | WDR87, APE1, MIP, Cx50/GJA3 and 8, CRYAA, |
| | CRYBB2, PRX, POLR3B, XRCC1, ZNF350, |
| | EPHA2, CRYGC |
| Cerebral Cavernous Malformation, | KRIT1, PDCD10 |
| Familial | |
| Cerebral Cavernous Malformations | CCM2, KRIT1 |
| Cerebral Cavernous Malformations 2 | CCM2 |
| Cerebral Cavernous Malformations 3 | KRIT1, PDCD10 |
| Cerebral Creatine Deficiency | GAMT |
| Syndrome 2 | |
| Cerebroretinal Microangiopathy with | CTC1 |
| Calcifications and Cysts 1 | |
| Ceroid Lipofuscinosis, Neuronal, 11 | GRN |
| Ceroid Lipofuscinosis, Neuronal, 3 | CLN3 |
| Ceroid Lipofuscinosis, Neuronal, 4 | CLN6 |
| Ceroid Lipofuscinosis, Neuronal, 6a | CLN5, CLN6, SMPD1, TPP1 |
| Ceroid Lipofuscinosis, Neuronal, 8, | CLN8 |
| Northern Epilepsy Variant | |
| Charge Syndrome | CHD7, PUF60 |
| Chorioretinal Scar | CHD7 |
| Choroidal Dystrophy, | PRPH2 |
| Central Areolar 2 | |
| Choroidal Dystrophy, | GUCY2D, PRPH2 |
| Central Areolar, 1 | |
| Choroideraemia | REP1 |
| Choroideremia | CHM, Rab escort protein |
| Cohen Syndrome | VPS13B |
| Coloboma of Iris | PRR12, TMEM67 |
| Coloboma of Macula | CDON, YAP1 |
| Coloboma, Ocular, Autosomal | SERPINA1 |
| Dominant | |
| Coloboma, Ocular, Autosomal | SALL2 |
| Recessive | |
| Colorblindness, Partial, Protan Series | OPN1LW |
| Cone Dystrophy | UPF1 |
| Cone Dystrophy 4 | PDE6C |
| Cone-Rod Dystrophy 15 | CDHR1 |
| Cone-Rod Dystrophy 2 | CABP4, CEP290, CERKL, CRB1, CRX, DRAM2, |
| | FAM161A, GUCY2D, PROM1, PRPH2, RPGR, |
| | TTLL5, USH2A |
| Cone-Rod Dystrophy 22 | TLCD3B |
| Cone-Rod Dystrophy 3 | ABCA4 |
| Cone-Rod Dystrophy 6 | ABCA4, ABHD12, CABP4, CERKL, CNGA3, |
| | GUCY2D, KCNV2, NMNAT1, SAG, SRD5A3, |
| | WDR19 |
| Cone-Rod Dystrophy 9 | ADAM9 |
| Congenital Dyserythropoietic Anemia | PNPLA1, TGM1 |
| Congenital Ptosis | FBN1 |
| Congenital Retinal Arteriovenous | MITF, MYO15A, USH2A |
| Communication | |
| Congenital Stationary Night Blindness | ABCA4, CABP4, CACNA1F, GRK1, NYX, |
| | PDE6B, TRPM1 |
| Contractural Arachnodactyly, | FBN2 |
| Congenital | |
| Corpus Callosum, Agenesis of | ARID1B, CDH2, CREBBP, DCC |
| D-Bifunctional Protein Deficiency | HSD17B4 |
| Deafness, Autosomal Recessive 18a | USH1C |
| Deafness, Autosomal Recessive 2 | MYO7A |
| Deafness, Autosomal Recessive 31 | WHRN |
| Diabetes and Deafness, Maternally | MT-TE, MT-TL1 |
| Inherited | |
| Donnai-Barrow Syndrome | LRP2 |
| Duchenne and Becker Muscular | DMD |
| Dystrophy | |
| Dyskeratosis Congenita | CTC1, RTEL1, TERC, TERT, TINF2 |
| Ectopia Lentis 2, Isolated, Autosomal | ADAMTSL4 |
| Recessive | |
| Enhanced S-Cone Syndrome | NR2E3, NRL |
| Exudative Vitreoretinopathy | FZD4, LRP5, RCBTB1, ZNF408 |
| Exudative Vitreoretinopathy 4 | LRP5 |
| Exudative Vitreoretinopathy 5 | TSPAN12 |
| Exudative Vitreoretinopathy 7 | CTNNB1 |
| Familial Retinoblastoma | RB1 |
| Focal Dermal Hypoplasia | PORCN |
| Fundus Albipunctatus | PRPH2, RDH5, RHO, RLBP1 |
| Fundus Dystrophy | ABCA4, ABHD12, ADGRV1, AHI1, AIPL1, |
| | ALMS1, ARL2BP, BBS1, BBS10, BBS12, BBS2, |
| | BBS9, BEST1, C1QTNF5, CABP4, CACNA1F, |
| | CC2D2A, CDH23, CDH3, CDHR1, CDKL5; RS1, |
| | CEP290, CEP78, CERKL, CFAP410, CHM, CLN3, |
| | CLRN1, CNGA1, CNGA3, CNGB1, CNGB3, |
| | COL18A1, COL2A1, CRB1, CRX, CYP4V2, |
| | DRAM2, EYS, FAM161A, FLVCR1, FRMD7, |
| | GRM6, GUCY2D, HGSNAT, HK1, IFT140, |
| | IFT172, IMPG1, IMPG2, INPP5E, IQCB1, KCNV2, |
| | KIF11, KIZ, LCA5, LRAT, LRP5, MERTK, MFRP, |
| | MYO7A, NMNAT1, NPHP4, NR2E3, NRL, OAT, |
| | OPA1, PCARE, PCDH15, PDE6A, PDE6B, PDE6C, |
| | PEX1, PRCD, PROM1, PRPF31, PRPF8, PRPH2, |
| | RBP3, RDH12, RDH5, RHO, RLBP1, RP1, RP1L1, |
| | RP2, RPE65, RPGR, RPGRIP1, RS1, SAG, |
| | SLC24A1, SNRNP200, SPATA7, TOPORS, |
| | TTLL5, TULP1, USH2A |
| Glaucoma | CALM2, MPP-7, Optineurin, LOX1, CYP1B1, |
| | CAV1/2, MYOC, PITX2, FOXC1, PAX6, CYP1B1, |
| | LTBP2 |
| Glaucoma 1, Open Angle, F | ASB10, OPTN |
| Glaucoma 3, Primary Congenital, a | CYP1B1 |
| Glaucoma, Primary Open Angle | LTBP2, MYOC, OPTN |
| Glucocorticoid Deficiency 4 with or | NNT |
| Without Mineralocorticoid Deficiency | |
| Glycogen Storage Disease Iv | GBE1 |
| Gm1 Gangliosidosis | GLB1 |
| Gyrate Atrophy of Choroid and Retina | OAT, RIMS1 |
| Hemophagocytic | UNC13D |
| Lymphohistiocytosis, Familial, 3 | |
| Hereditary Spastic Paraplegia | ALDH18A1, AP4B1, AP4M1, AP5Z1, ATL1, |
| | CYP2U1, CYP7B1, DDHD2, FA2H, GBA2, KIF1C, |
| | NIPA1, PNPLA6, POLG, REEP1, SACS, SPAST, |
| | SPG11, SPG21, SPG7, ZFYVE26 |
| Hermansky-Pudlak Syndrome | BLOC1S6, HPS1, HPS3, HPS4, HPS5, HPS6, |
| | RUNX1 |
| Hermansky-Pudlak Syndrome 8 | BLOC1S3 |
| Heterochromia Iridis | MITF |
| Hyperornithinemia- | SLC25A15 |
| Hyperammonemia-Homocitrullinuria | |
| Syndrome | |
| Iminoglycinuria | SLC36A2 |
| Incontinentia Pigmenti | IKBKG |
| Inherited Retinal Disorder | VPS13B |
| Intraocular Pressure Quantitative Trait | ZEB1 |
| Locus | |
| Isolated Ectopia Lentis | FBN1 |
| Jalili Syndrome | CNNM4 |
| Joubert Syndrome 3 | AHI1 |
| Juvenile Retinoschisis | ATP1A3 |
| Kearns-Sayre Syndrome | MT-TL1, MT-TY |
| Keratitis, Neurotrophic | NGF |
| Keratoconjunctivitis, Atopic | |
| Keratoconjunctivitis, Vernal | |
| Keratomalacia | CEP290 |
| Knobloch Syndrome | COL18A1 |
| Kufor-Rakeb Syndrome | ATP13A2 |
| Late-Onset Retinal Degeneration | C1QTNF5 |
| Leber congenital amaurosis (LCA) | RPE65, GUCY2D, KIR7.1 |
| Leber Congenital Amaurosis 1 | CRB1, GUCY2D, LCA5, RPGRIP1, TULP1 |
| Leber Congenital Amaurosis 10 | CEP290 |
| Leber Congenital Amaurosis 12 | RD3 |
| Leber Congenital Amaurosis 13 | RDH12 |
| Leber Congenital Amaurosis 14 | LRAT |
| Leber Congenital Amaurosis 15 | TULP1 |
| Leber Congenital Amaurosis 16 | KCNJ13 |
| Leber Congenital Amaurosis 17 | GDF6 |
| Leber Congenital Amaurosis 19 | USP45 |
| Leber Congenital Amaurosis 2 | RPE65 |
| Leber Congenital Amaurosis 3 | SPATA7 |
| Leber Congenital Amaurosis 4 | AIPL1 |
| Leber Congenital Amaurosis 5 | LCA5 |
| Leber Congenital Amaurosis 6 | RPGRIP1 |
| Leber Congenital Amaurosis 7 | CRX |
| Leber Congenital Amaurosis 8 | CRB1 |
| Leber Congenital Amaurosis 9 | NMNAT1 |
| Leber hereditary optic neuropathy | ND4, MT-ND1 |
| (LHON) | |
| Leber Hereditary Optic Neuropathy, | MT-ATP6, MT-ND1, MT-ND2, MT-ND4, MT- |
| Modifier of | ND4L, MT-ND5, MT-ND6, NDUFS2 |
| Leber Plus Disease | ABCA4, AHI1, AIPL1, ALMS1, CEP290, CRB1, |
| | GRM6, GUCY2D, INPP5E, IQCB1, LCA5, LRAT, |
| | NDUFAF5, NMNAT1, PDE6B, PEX1, RDH12, |
| | RP2, RPE65, RPGRIP1, SLC38A8, SPATA7, |
| | TULP1, USH2A |
| Liberfarb Syndrome | PISD |
| Long-Chain 3-Hydroxyacyl-Coa | HADHA, HADHB, HMGCL |
| Dehydrogenase Deficiency | |
| Macular Degeneration, Age-Related, | CFI |
| 13 | |
| Macular Degeneration, Age-Related, | ABCA4, MT-TL1 |
| 2 | |
| Macular Degeneration, diabetic | VEGFA, VEGFC |
| Macular Degeneration, Oedema | VEGFA, VEGFC |
| Macular Dystrophy, Patterned, 1 | PRPH2 |
| Macular Dystrophy, Vitelliform, 2 | BEST1, IMPG2, PRPH2 |
| Macular Dystrophy, Vitelliform, 3 | PRPH2 |
| Macular Dystrophy, Vitelliform, 4 | IMPG1 |
| Macular Dystrophy, Vitelliform, 5 | IMPG2 |
| Marfan Syndrome | FBN1, TGFBR2, MTHFR, MTR, MTRR |
| Maturity-Onset Diabetes of the Young | APPL1, GCK, HNF1A, HNF1B, KCNJ11, MLKL |
| Melanoma, Uveal | SF3B1 |
| Mental Retardation, Truncal Obesity, | INPP5E |
| Retinal Dystrophy, and Micropenis | |
| Syndrome | |
| Methylmalonic Acidemia with | MMACHC |
| Homocystinuria | |
| Microcephaly and Chorioretinopathy, | TUBGCP6 |
| Autosomal Recessive, 1 | |
| Microcephaly and Chorioretinopathy, | TUBGCP4 |
| Autosomal Recessive, 3 | |
| Microcephaly with or Without | KIF11 |
| Chorioretinopathy, Lymphedema, or | |
| Mental Retardation | |
| Microphthalmia | ALDH1A3 |
| Microphthalmia, Isolated 2 | VSX2 |
| Microphthalmia, Isolated 3 | RAX |
| Microphthalmia, Isolated 5 | MFRP |
| Microphthalmia, Isolated 7 | GDF3 |
| Microphthalmia, Isolated, with | RBP4 |
| Coloboma 10 | |
| Microphthalmia, Isolated, with | SHH |
| Coloboma 5 | |
| Microphthalmia, Isolated, with | GDF6 |
| Coloboma 6 | |
| Microphthalmia, Isolated, with | ABCB6 |
| Coloboma 7 | |
| Microphthalmia, Isolated, with | TENM3 |
| Coloboma 9 | |
| Microphthalmia, Syndromic 9 | STRA6, WNT7B |
| Mrcs Syndrome | BEST1 |
| Mucolipidosis Iv | MCOLN1 |
| Multiple Congenital Anomalies- | PIGT |
| Hypotonia-Seizures Syndrome 3 | |
| Muscular Dystrophy, Becker Type | DMD |
| Muscular Dystrophy- | POMGNT1 |
| Dystroglycanopathy, Type a, 3 | |
| Muscular Dystrophy- | POMGNT1 |
| Dystroglycanopathy, Type B, 3 | |
| Muscular Dystrophy- | POMGNT1 |
| Dystroglycanopathy, Type C, 3 | |
| Myeloperoxidase Deficiency | MPO |
| Myopia | HGF, C-MET, UMODL1, MMP-1/2, PAX6, CBS, |
| | MTHFR, IGF-1, UHRF1BPIL, PTPRR, PPFIA2, |
| | P4HA2 |
| Myopia 2, Autosomal Dominant | CNP |
| Myopia 21, Autosomal Dominant | ZNF644 |
| Myopia 24, Autosomal Dominant | SLC39A5 |
| Myopia 25, Autosomal Dominant | P4HA2 |
| Myopia 26, X-Linked, Female- | ARR3 |
| Limited | |
| Myopia 27, Autosomal Dominant | CPSF1 |
| Myopia 6 | NCAPH2; SCO2; TYMP, TYMP; NCAPH2; SCO2 |
| Myotonia Congenita, Autosomal | CLCN1 |
| Recessive | |
| Neonatal Adrenoleukodystrophy | PEX1 |
| Neurofibromatosis, Type Ii | NF2 |
| Neuronal Ceroid Lipofuscinosis | CLN3, CLN5, CLN6, CLN8, CTSD, PPT1, TPP1 |
| Neuronal Ceroid-Lipofuscinoses | PPT1 |
| Neuroocular Syndrome | PRR12 |
| Neuropathy, Ataxia, and Retinitis | MT-ATP6 |
| Pigmentosa | |
| Neuropathy, Ischaemic Optic | |
| Nevus of Ota | HRAS |
| Newfoundland Rod-Cone Dystrophy | RLBP1 |
| Night Blindness, Congenital | RHO |
| Stationary, Autosomal Dominant 1 | |
| Night Blindness, Congenital | GPR179 |
| Stationary, Type 1e | |
| Night Blindness, Congenital | CACNA1F |
| Stationary, Type 2a | |
| Nonsyndromic Hearing Loss | GJB2, GJB3, GJB6, SOHLH1 |
| Noonan Syndrome with Multiple | PTPN11, RAF1 |
| Lentigines | |
| Norrie Disease | NDP |
| Oculocutaneous Albinism | SLC24A5, TYR |
| Oculocutaneous Albinism, Type Viii | DCT |
| Oguchi Disease 1 | SAG |
| Oliver-Mcfarlane Syndrome | PNPLA6 |
| Opitz Gbbb Syndrome | MID1, SPECC1L |
| Optic Atrophy 1 | MT-ATP8 |
| Optic Atrophy 10 with or Without | RTN4IP1 |
| Ataxia, Mental Retardation, and | |
| Seizures | |
| Optic Atrophy 12 | AFG3L2 |
| Optic Atrophy 13 with Retinal and | SSBP1 |
| Foveal Abnormalities | |
| Optic Atrophy 3, Autosomal | DNAJC19, MT-ND1, OPA3 |
| Dominant | |
| Optic Atrophy 7 with or Without | TMEM126A |
| Auditory Neuropathy | |
| Optic Atrophy 9 | ACO2 |
| Optic Atrophy with or Without | OPA1 |
| Deafness, Ophthalmoplegia, | |
| Myopathy, Ataxia, and Neuropathy | |
| Optic Nerve Glioma | NF1 |
| Optic Nerve Hypoplasia, Bilateral | ATOH7, COL4A1, PAX6 |
| Optical Neuropathy | Complex I or ND genes, OPA1, RPE65 |
| Osteopetrosis, Autosomal Recessive 3 | CA2 |
| Osteoporosis-Pseudoglioma | LRP5 |
| Syndrome | |
| Papillorenal Syndrome | PAX2 |
| Pathologic Nystagmus | CEP290 |
| Pendred Syndrome | FOXI1, SLC26A4 |
| Peroxisomal Disease | PEX1 |
| Persistent Hyperplastic Primary | ATOH7 |
| Vitreous, Autosomal Recessive | |
| Pigmented Paravenous Chorioretinal | CRB1, MT-TH, PRPH2 |
| Atrophy | |
| Polypoidal Choroidal Vasculopathy | C2, C3, CFH, SERPING1, PEDF, ARMS2-HTRA1, |
| | FGD6, ABCG1, LOC387715, CETP |
| Posterior Column Ataxia with | FLVCR1 |
| Retinitis Pigmentosa | |
| Primary Congenital Glaucoma | CYP1B1 |
| Primary Hyperoxaluria | AGXT, GRHPR |
| Progressive External Ophthalmoplegia | MT-TL1, POLG |
| with Mitochondrial Dna Deletions, | |
| Autosomal Dominant 1 | |
| Progressive External Ophthalmoplegia | SLC25A4 |
| with Mitochondrial Dna Deletions, | |
| Autosomal Dominant 2 | |
| Progressive External Ophthalmoplegia | TWNK |
| with Mitochondrial Dna Deletions, | |
| Autosomal Dominant 3 | |
| Progressive External Ophthalmoplegia | POLG2 |
| with Mitochondrial Dna Deletions, | |
| Autosomal Dominant 4 | |
| Progressive External Ophthalmoplegia | POLG, TWNK |
| with Mitochondrial Dna Deletions, | |
| Autosomal Recessive 1 | |
| Progressive Myoclonus Epilepsy | CSTB, EPM2A, GOSR2, SCARB2 |
| Proliferative Vasculopathy and | FLVCR2 |
| Hydranencephaly-Hydrocephaly | |
| Syndrome | |
| Prolonged Electroretinal Response | F2, KCNH2 |
| Suppression | |
| Pseudoxanthoma Elasticum | ABCC2, ABCC6 |
| Pyruvate Carboxylase Deficiency | PC |
| Rare Genetic Deafness | ACTG1, ADGRV1, ATP6V1B1, CDC14A, CDH23, |
| | CLRN1, COL4A5, DIAPH1, EDNRB, ESRRB, |
| | EYA1, GIPC3, GJB2, GPSM2, GRXCR1, KCNQ4, |
| | LOXHD1, LRTOMT, MARVELD2, MITF, |
| | MSRB3, MT-RNR1, MT-TS1, MYO15A, MYO6, |
| | MYO7A, OTOA, OTOF, OTOG, OTOGL, PAX3, |
| | PCDH15, PJVK, POU3F4, SERPINB6, SLC26A4, |
| | SOX10, STRC, SYNE4, TECTA, TMC1, |
| | TMPRSS3, USH1C, USH2A, WFS1, WHRN |
| Refsum Disease, Classic | PEX7, PHYH |
| Retinal Arterial Macroaneurysm with | IGFBP7 |
| Supravalvular Pulmonic Stenosis | |
| Retinal Arteries, Tortuosity of | COL4A1 |
| Retinal Cone Dystrophy 3b | KCNV2 |
| Retinal Dystrophy and Obesity | TUB |
| Retinal Dystrophy, Iris Coloboma, | RBP4 |
| and Comedogenic Acne Syndrome | |
| Retinal Vascular Occlusion | BBS9 |
| Retinitis | RLBP1 |
| Retinitis Pigmentosa | ABCA4, ADGRV1, AHI1, ALMS1, BBS1, |
| | BBS1; ZDHHC24, BBS2, BBS7, CACNA1F, CDH3, |
| | CDHR1, CEP290, CERKL, CHM, CLRN1, CNGA1, |
| | CNGB1, CRB1, EYS, FAM161A, FLVCR1, |
| | GUCY2D, HGSNAT, IFT140, IFT172, IMPG2, |
| | LCA5, MAK, MERTK, MYO7A, NR2E3, NRL, |
| | P3H2, PANK2, PCARE, PDE6A, PDE6B, PPT1, |
| | PRCD, PROM1, PRPF31, PRPF8, PRPH2, |
| | RCBTB1, RDH12, RHO, RLBP1, RP1, RP1L1, |
| | RP2, RPE65, RPGR, RPGRIP1, SLC24A1, |
| | SNRNP200, SPATA7, TTLL5, TULP1, USH2A, |
| | VPS13B, VSX2 |
| Retinitis Pigmentosa (RP, including | RLBP1 |
| RLBP1) | |
| Retinitis Pigmentosa 1 | RP1 |
| Retinitis Pigmentosa 10 | IMPDH1 |
| Retinitis Pigmentosa 11 | PRPF31 |
| Retinitis Pigmentosa 12 | CRB1 |
| Retinitis Pigmentosa 13 | PRPF8 |
| Retinitis Pigmentosa 14 | TULP1 |
| Retinitis Pigmentosa 19 | ABCA4 |
| Retinitis Pigmentosa 2 | RP2 |
| Retinitis Pigmentosa 20 | RPE65 |
| Retinitis Pigmentosa 23 | OFD1 |
| Retinitis Pigmentosa 25 | EYS |
| Retinitis Pigmentosa 26 | CERKL |
| Retinitis Pigmentosa 27 | NRL |
| Retinitis Pigmentosa 28 | FAM161A |
| Retinitis Pigmentosa 3 | RP2, RPGR |
| Retinitis Pigmentosa 33 | SNRNP200 |
| Retinitis Pigmentosa 36 | CYGB; PRCD, PRCD |
| Retinitis Pigmentosa 37 | NR2E3 |
| Retinitis Pigmentosa 38 | MERTK |
| Retinitis Pigmentosa 39 | USH2A |
| Retinitis Pigmentosa 4 | CFAP418, RHO |
| Retinitis Pigmentosa 40 | PDE6B, PDE6B; PDE6B-AS1 |
| Retinitis Pigmentosa 41 | PROM1 |
| Retinitis Pigmentosa 42 | KLHL7 |
| Retinitis Pigmentosa 43 | PDE6A |
| Retinitis Pigmentosa 45 | CNGB1 |
| Retinitis Pigmentosa 47 | SAG |
| Retinitis Pigmentosa 49 | CNGA1 |
| Retinitis Pigmentosa 54 | PCARE |
| Retinitis Pigmentosa 55 | ARL6 |
| Retinitis Pigmentosa 56 | IMPG2 |
| Retinitis Pigmentosa 59 | DHDDS |
| Retinitis Pigmentosa 6 | RPGR |
| Retinitis Pigmentosa 62 | MAK |
| Retinitis Pigmentosa 66 | RBP3 |
| Retinitis Pigmentosa 69 | KIZ |
| Retinitis Pigmentosa 7 | PRPH2 |
| Retinitis Pigmentosa 76 | POMGNT1 |
| Retinitis Pigmentosa 77 | REEP6 |
| Retinitis Pigmentosa 85 | AHR |
| Retinitis Pigmentosa 88 | RP1L1 |
| Retinitis Pigmentosa 90 | IDH3 A |
| Retinitis Pigmentosa 91 | ABCA4, CRX, IMPG1 |
| Retinitis Pigmentosa-Deafness | MT-TS2 |
| Syndrome | |
| Retinopathy (e.g., diabetic) | |
| Retinoschisis 1, X-Linked, Juvenile | CDKL5; RS1, RS1 |
| Revesz Syndrome | TINF2 |
| Rhizomelic Chondrodysplasia | GNPAT |
| Punctata, Type 2 | |
| Saul-Wilson Syndrome | COG4 |
| Senior-Loken Syndrome 1 | IQCB1, NPHP1, SDCCAG8, TRAF3IP1, WDR19 |
| Severe Early-Childhood-Onset Retinal | ABCA4, ALMS1 |
| Dystrophy | |
| Short Stature, Optic Nerve Atrophy, | NBAS |
| and Pelger-Huet Anomaly | |
| Short-Rib Thoracic Dysplasia 5 with | WDR19 |
| or Without Polydactyly | |
| Short-Rib Thoracic Dysplasia 9 with | IFT140 |
| or Without Polydactyly | |
| Sjogren-Larsson Syndrome | ALDH3A2 |
| Spastic Ataxia, Charlevoix-Saguenay | SACS |
| Type | |
| Spastic Paraplegia 15, Autosomal | ZFYVE26 |
| Recessive | |
| Spondylometaphyseal Dysplasia, | CFAP410 |
| Axial | |
| Stargardt disease | ABCA1, ABCA4, CRB1 |
| Stargardt Disease | ABCA4, BEST1, COL2A1, CRB1, CRX, PRPH2, |
| | TULP1 |
| Stargardt Disease 3 | ELOVL4 |
| Stickler Syndrome | COL9A2 |
| Stickler Syndrome, Type I, | COL2A1 |
| Nonsyndromic Ocular | |
| Sveinsson Chorioretinal Atrophy | SEMA4A |
| Syndromic Rod-Cone Dystrophy | EPG5, KIF11 |
| Tay-Sachs Disease | HEXA |
| Temtamy Syndrome | C12orf57 |
| Tetrahydrobiopterin Deficiency | ATP1A3 |
| Tietz Albinism-Deafness Syndrome | MITF |
| Tumoral Calcinosis, | FGF23 |
| Hyperphosphatemic, Familial, 1 | |
| Usher Syndrome | ADGRV1, CEP250, CLRN1, MYO7A, PROM1, |
| | USH1C, USH1G, USH2A |
| Usher Syndrome 2A | USH2A |
| Usher Syndrome Type 2 | ADGRV1, CDH23, USH2A |
| Usher Syndrome, Type I | CDH23, MYO7A, PCDH15, USH1C |
| Usher Syndrome, Type I (USH1) | MYO7A |
| Usher Syndrome, Type Ic | USH1C |
| Usher Syndrome, Type Id | CDH23, PCDH15 |
| Usher Syndrome, Type if | PCDH15 |
| Usher Syndrome, Type Ig | PCDH15, USH1G |
| Usher Syndrome, Type Iia | USH2A |
| Usher Syndrome, Type lic | ADGRV1 |
| Usher Syndrome, Type Iid | WHRN |
| Usher Syndrome, Type Iiia | CLRN1 |
| Usher Syndrome, Type Iv | ARSG |
| Uveal Melanoma | PTEN, BAP1, GNAQ, GNA11, DDEF1, SF3B1, |
| | EIF1AX, CDKN2A, p14ARF, HERC2/OCA2 |
| Uveitis | |
| Vascular Dementia | HTRA1, NOTCH3 |
| Vasculopathy, Retinal, with Cerebral | TREX1 |
| Leukoencephalopathy and Systemic | |
| Manifestations | |
| Von Hippel-Lindau Syndrome | VHL |
| Wagner Vitreoretinopathy | VCAN |
| Walker-Warburg Syndrome | FKRP, FKTN, POMT1 |
| Weaver Syndrome | EZH2 |
| Wet AMD | Anti-VEGF antibody |
| Wet AMD, Dry AMD | NRP1 |
| X-linked retinitis pigmentosa (X- | RPGR |
| linked RP) | |
| X-linked Retinoschisis | RS1 |

In some embodiments, the therapeutic polynucleotide encodes a therapeutic RNA. In some embodiments the therapeutic polynucleotide encodes an RNA, such as a guide RNA (including an engineered or synthetic guide RNA) for genome editing or for RNA editing. The guide RNA may target a gene, such as those provided in TABLE 1 or encoding a protein nucleotide provided in TABLE 1, to promote editing of the target gene. In some embodiments, editing of the target gene may treat a condition in a subject, such as a condition provided in TABLE 1.

In some embodiments, the therapeutic polynucleotide encodes a tRNA or a modified tRNA (engineered or synthetic tRNA). For example, the tRNA or modified tRNA can be a suppressor tRNA. The suppressor tRNA can be engineered to have an anticodon region that recognizes a stop codon, such as any premature stop codon (opal, ochre, or amber stop codons). A suppressor tRNA may target a premature stop codon in a gene, such as those provided in TABLE 1 or encoding a protein nucleotide provided in TABLE 1, to promote readthrough of the gene. In some embodiments, suppressing the premature stop codon may treat a condition in a subject, such as a condition provided in TABLE 1.

In some embodiments, the therapeutic polynucleotide (e.g., a therapeutic RNA, a tRNA, or a genome modifying entity) can target a gene listed in TABLE 1 or any gene associated with an ocular disease, such as achromatopsia, macular degeneration (e.g., wet AMD or dry AMD), cataracts, choroideremia, glaucoma, optical neuropathy, Marfan syndrome, myopia, polypoidal choroidal vasculopathy, retinitis pigmentosa, Stargardt disease, Usher syndrome, Leber congenital amaurosis, Leber hereditary optical neuropathy, Uveal melanoma, or X-linked retinoschisis, or any genetic disease affecting an ocular tissue. In some embodiments, the targeted gene may be CNGB3, NOS2A, CFH, CF, C2, C3, CFB, HTRA1/LOC, MMP-9, TIMP-3, SLC16A8, GEMIN4, CYP51A1, RIC1, TAPT1, TAF1A, WDR87, APE1, MIP, Cx50/GJA3 and 8, CRYAA, CRYBB2, PRX, POLR3B, XRCC1, ZNF350, EPHA2, REP1, CALM2, MPP-7, Optineurin, LOX1, CYP1B1, CAV1/2, MYOC, PITX2, FOXC1, PAX6, LTBP2, Complex I or ND genes, OPA1, RPE65, FBN1, TGFBR2, MTHFR, MTR, MTRR, HGF, C-MET, UMODL1, MMP-1/2, CBS, IGF-1, UHRF1BP1L, PTPRR, PPFIA2, P4HA2, SERPING1, PEDF, ARMS2-HTRA1, FGD6, ABCG1, LOC387715, CETP, NRL, RDH12, PRPH2 (RDS), RHO, RPGR, NR2E3, IMPDH1, CRX, HK1, IMPDH2, SNRNP200, PRPF3, AGBL5, ABCA1, ABCA4, CRB1, USH2A, NRP1, ND4, RLBP1, PTEN, BAP1, GNAQ, GNA11, DDEF1, SF3B1, EIF1AX, CDKN2A, p14ARF, HERC2/OCA2, VEGF, RS1, a fragment any of these, or any combination thereof. In some embodiments, the therapeutic polynucleotide is a gene therapy payload (e.g., a transgene) and, thus, may itself be one of the genes listed in TABLE 1 or any gene associated with an ocular disease, such as achromatopsia, macular degeneration (e.g., wet AMD or dry AMD), cataracts, choroideremia, glaucoma, optical neuropathy, Marfan syndrome, myopia, polypoidal choroidal vasculopathy, retinitis pigmentosa, Stargardt disease, Usher syndrome, Leber congenital amaurosis, Leber hereditary optical neuropathy, Uveal melanoma, or X-linked retinoschisis, or any genetic disease. In some embodiments, the transgene may be CNGB3, NOS2A, CFH, CF, C2, C3, CFB, HTRA1/LOC, MMP-9, TIMP-3, SLC16A8, GEMIN4, CYP51A1, RIC1, TAPT1, TAF1A, WDR87, APE1, MIP, Cx50/GJA3 and 8, CRYAA, CRYBB2, PRX, POLR3B, XRCC1, ZNF350, EPHA2, REP1, CALM2, MPP-7, Optineurin, LOX1, CYP1B1, CAV1/2, MYOC, PITX2, FOXC1, PAX6, LTBP2, Complex I or ND genes, OPA1, RPE65, FBN1, TGFBR2, MTHFR, MTR, MTRR, HGF, C-MET, UMODL1, MMP-1/2, CBS, IGF-1, UHRF1BP1L, PTPRR, PPFIA2, P4HA2, SERPING1, PEDF, ARMS2-HTRA1, FGD6, ABCG1, LOC387715, CETP, NRL, RDH12, PRPH2 (RDS), RHO, RPGR, NR2E3, IMPDH1, CRX, HK1, IMPDH2, SNRNP200, PRPF3, AGBL5, ABCA1, ABCA4, CRB1, USH2A, NRP1, ND4, RLBP1, PTEN, BAP1, GNAQ, GNA11, DDEF1, SF3B1, EIF1AX, CDKN2A, p14ARF, HERC2/OCA2, VEGF, RS1, a fragment any of these, or any combination thereof. In some embodiments, the transgene may encode a protein that targets a gene, a protein, or a protein encoded by a gene provided in TABLE 1, a fragment any of these, or any combination thereof.

An engineered AAV comprising a VP capsid polypeptide comprising an ocular tissue-tropic 581 to 589 region may be used to deliver a payload to treat an ocular condition, or a condition of the eye. For example, an engineered AAV comprising a VP capsid polypeptide comprising a 581 to 589 region conferring ocular tissue tropism may be used to treat an ocular condition (e.g., achromatopsia, macular degeneration (e.g., wet AMD or dry AMD), cataracts, choroideremia, glaucoma, optical neuropathy, Marfan syndrome, myopia, polypoidal choroidal vasculopathy, retinitis pigmentosa, Stargardt disease, Usher syndrome, Leber congenital amaurosis, Leber hereditary optical neuropathy, Uveal melanoma, or X-linked retinoschisis). The 581 to 589 region of the VP capsid polypeptide may have a sequence of any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. In some embodiments, the engineered AAV comprising a VP capsid polypeptide comprising an ocular tissue-tropic 581 to 589 region may be used to deliver a transgene encoding a protein associated with an ocular condition or that targets a protein associated with an ocular condition (e.g., a protein encoded by CNGB3, NOS2A, CFH, CF, C2, C3, CFB, HTRA1/LOC, MMP-9, TIMP-3, SLC16A8, GEMIN4, CYP51A1, RIC1, TAPT1, TAF1A, WDR87, APE1, MIP, Cx50/GJA3 and 8, CRYAA, CRYBB2, PRX, POLR3B, XRCC1, ZNF350, EPHA2, REP1, CALM2, MPP-7, Optineurin, LOX1, CYP1B1, CAV1/2, MYOC, PITX2, FOXC1, PAX6, LTBP2, Complex I or ND genes, OPA1, RPE65, FBN1, TGFBR2, MTHFR, MTR, MTRR, HGF, C-MET, UMODL1, MMP-1/2, CBS, IGF-1, UHRF1BP1L, PTPRR, PPFIA2, P4HA2, SERPING1, PEDF, ARMS2-HTRA1, FGD6, ABCG1, LOC387715, CETP, NRL, RDH12, PRPH2 (RDS), RHO, RPGR, NR2E3, IMPDH1, CRX, HK1, IMPDH2, SNRNP200, PRPF3, AGBL5, ABCA1, ABCA4, CRB1, USH2A, NRP1, ND4, RLBP1, PTEN, BAP1, GNAQ, GNA11, DDEF1, SF3B1, EIF1AX, CDKN2A, p14ARF, HERC2/OCA2, VEGF, RS1). For example, an engineered AAV comprising a VP capsid polypeptide comprising a retina tissue-tropic 581 to 589 region (e.g., any one of SEQ ID NO: 14-SEQ ID NO: 2013 or SEQ ID NO: 2514-SEQ ID NO: 4513) may be used to deliver a payload encoding a therapeutic protein associated with retinitis pigmentosa (e.g., NRL, RDH12, PRPH2 (RDS), RHO, RPGR, SNRNP200, NR2E3, IMPDH1, CRX, HK1, IMPDH2, SNRNP200, PRPF3, or AGBL5) to retina tissue to treat retinitis pigmentosa. In another example, an engineered AAV comprising a VP capsid polypeptide comprising an RPE tissue-tropic 581 to 589 region (e.g., any one of SEQ ID NO: 2014-SEQ ID NO: 2513 or SEQ ID NO: 4514-SEQ ID NO: 5013) may be used to deliver a payload encoding a therapeutic protein associated with dry age-related macular degeneration to RPE tissue to treat dry age-related macular degeneration.

In some embodiments, the therapeutic polynucleotide encodes genome modifying entities. For example, a genome modifying entity may be a DNA editing enzyme, an RNA editing enzyme, a transcriptional activator, or a transcriptional repressor. The DNA editing enzyme may be any DNA editing enzyme, including any CRISPR/Cas systems, meganucleases, zinc-finger nucleases, (ZFNs), TALE Nucleases (TALENs and megaTALENS). The CRISPR/Cas system can be a Cas3, Cas8, Cas10, Cas9, Cas4, Cas12, or Cas13. The RNA editing enzyme may be ADAR. In some embodiments, the ADAR is a human ADAR1 or human ADAR2. The transcriptional activator may be VP64. A transcriptional repressor may be KRAB. Such genome modifying entities may target any gene listed in TABLE 1 for editing.

In some embodiments, the present disclosure provides for rAAV virions having an engineered AAV VP capsid polypeptide (e.g., comprising a 581 to 589 region variant), where the virion encapsidates any one of or any combination of the therapeutic payloads disclosed herein. In some embodiments, multiple copies of the therapeutic payload are encapsidated. The 581 to 589 region of the engineered AAV VP capsid polypeptide may have a sequence of any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013.

In some embodiments, the therapeutic polynucleotide is a polynucleotide capable of serving as a homology template for homology-directed repair. In some embodiments, the therapeutic polynucleotide may be a guide polynucleotide for a CRISPR/Cas system or an ADAR enzyme. For example, the therapeutic polynucleotide may be a CRISPR/Cas guide RNA or an ADAR guide RNA.

In some embodiments, an rAAV virion of the present disclosure, having any of the engineered AAV VP capsid polypeptide sequences disclosed herein, comprises a vector genome, the vector genome comprising a detectable polynucleotide or payload. In further embodiments, said payload may be under control of regulatory sequences that direct expression in infected human cells. In some embodiments, the payload may be under control of a cell type- or tissue type-specific promoter to direct expression in a target cell type or target tissue type. For example, expression of the payload may be under control of a retinal-specific promoter to promote retinal-specific expression, a retinal pigment epithelium-specific promoter to promote retinal pigment epithelium-specific expression, a choroid-specific promoter to promote choroid-specific expression, a photoreceptor-specific promoter to promote photoreceptor-specific expression, or an optic nerve-specific promoter to promote optic nerve-specific expression. Examples of detectable polynucleotides include, but are not limited to, any genetically encodable detectable moiety. For example, a genetically encodable detectable moiety may be a fluorescent protein such as EGFP, GFP, YFP, RFP, CFP, or any variants thereof. In some embodiments, the present disclosure provides for rAAV virions having an engineered AAV VP capsid polypeptide, where the virion encapsidates any one of or any combination of the detectable payloads disclosed herein. In some embodiments, multiple copies of the detectable payload are encapsidated.

In some embodiments, the present disclosure provides for rAAV virions having an engineered AAV VP capsid polypeptide, where the virion encapsidates any one of or any combination of the therapeutic payloads and detectable payloads disclosed herein. For example, an rAAV of the present disclosure having an engineered AAV VP capsid polypeptide may encapsidate a transgene and a fluorescent protein. As another example, an rAAV of the present disclosure having an engineered AAV VP capsid polypeptide may encapsidate a therapeutic RNA (e.g., a guide RNA) and a fluorescent protein.

Delivering a payload (e.g., a payload encoding a therapeutic polypeptide or a therapeutic polynucleotide) using the ocular tissue specific AAVs described herein may produce lower toxicity in a subject compared to non-specific AAVs (e.g., AAVs comprising a VP1 polypeptide of SEQ ID NO: 1). Tissue specific AAVs (e.g., ocular tissue specific AAVs) may be effective at lower doses compared to non-specific AAVs since a larger fraction of the administered AAVs infect the relevant tissue (e.g., ocular tissue). As a result, a therapeutically effective dose of tissue specific AAVs may be lower than a therapeutically effective dose of non-specific AAVs, leading to lower toxicity due administering a lower dose. For example, administration of a therapeutically effective dose of tissue specific AAVs may result in lower liver toxicity than administration of a therapeutically effective dose of non-specific AAVs. Administration of a lower dose may also lead to lower production of neutralizing antibodies in the subject. Neutralizing antibodies may decrease the efficacy of the AAV therapy by inhibiting infection of the target tissue or may cause severe side effects in the subject due to the immune response. Additionally, tissue specific AAVs (e.g., ocular tissue specific) may produce fewer off-target effects compared to non-specific AAVs when administered at the same dose since fewer of the AAVs infect off target tissues (e.g., non-targeted ocular tissues such as aqueous humor or vitreous humor). Off-target effects may include increased gene expression in an off-target tissue, decreased gene expression in an off-target tissue, gene editing in an off-target tissue, immune response, or liver toxicity.

### In Vivo Selected VP Polypeptides

In a further aspect, engineered (synonymously, recombinant) adeno-associated virus (AAV) VP capsid polypeptides identified using the methods described herein are provided.

In some embodiments, the engineered adeno-associated virus (AAV) viral protein (VP) capsid polypeptide has an amino acid sequence at least 70% identical to SEQ ID NO: 1, wherein the engineered AAV VP capsid polypeptide has at least one substitution as compared to SEQ ID NO: 1 in the 581 to 589 region, corresponding to residue 581 to residue 589 of SEQ ID NO: 1, inclusive, wherein the capsid polypeptide is capable of assembling into a recombinant AAV virion (rAAV), and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8. In some embodiments, the VP capsid polypeptide comprises one or more amino acid substitutions relative to SEQ ID NO: 1 in the region from residue 581 to residue 589, inclusive. The VP capsid polypeptide may comprise a sequence of SEQ ID NO: 2, wherein the 581 to 589 region has a sequence conferring ocular tissue tropism or preference.

In some embodiments, the engineered adeno-associated virus (AAV) viral protein (VP) capsid polypeptide is ocular tissue-tropic and has an amino acid sequence at least 70% identical to SEQ ID NO: 1, wherein the engineered AAV VP capsid polypeptide has at least one substitution as compared to SEQ ID NO: 1 in the 581 to 589 region, corresponding to residue 581 to residue 589 of SEQ ID NO: 1, inclusive, wherein the capsid polypeptide is capable of assembling into a recombinant AAV virion (rAAV), wherein the at least one substitution confers higher tropism for an ocular tissue on the rAAV as compared to an rAAV virion having an AAV5 VP capsid polypeptide of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8. In some embodiments, the ocular tissue-tropic VP capsid polypeptide comprises one or more amino acid substitutions relative to SEQ ID NO: 1 in the region from residue 581 to residue 589, inclusive.

In particular embodiments, the engineered adeno-associated virus (AAV) viral protein (VP) capsid polypeptide (e.g., an ocular tissue-tropic VP capsid polypeptide) has an amino acid sequence at least 75%, 77.7%, 80%, 85%, 88.8%, 90%, 91%, 92%, 93%, 94%, 95%, 96%, 97%, 98% identical to the sequence of SEQ ID NO: 1.

In some embodiments, the AAV VP capsid polypeptide is an ocular tissue-tropic capsid polypeptide and has an amino acid sequence of SEQ ID NO: 2, wherein X¹, X², X³, X⁴, X⁵, X⁶, X⁷, X⁸, and X⁹ are each independently selected from any amino acid, wherein the capsid polypeptide is capable of assembling into a recombinant AAV virion (rAAV), wherein the at least one substitution confers higher tropism for an ocular tissue on the rAAV as compared to an rAAV virion having an AAV5 VP capsid polypeptide of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8, optionally with further mutations elsewhere in the VP capsid polypeptide. In some embodiments, X¹X²X³X⁴X⁵X⁶X⁷X⁸X⁹ (SEQ ID NO: 5014) of the ocular tissue-tropic VP capsid polypeptide correspond to a 581 to 589 region conferring ocular tissue tropism or preference.

In some embodiments, the engineered adeno-associated virus (AAV) viral protein (VP) capsid polypeptide (e.g., an ocular tissue-tropic VP capsid polypeptide) has an amino acid sequence of SEQ ID NO: 2, wherein amino acid residues X¹, X², X³, X⁴, X⁵, X⁶, X⁷, X⁸, and X⁹ are each independently selected from A, R, N, D, C, E, Q, G, H, I, L, K, M, F, P, S, T, W, Y, and V; wherein the engineered AAV VP capsid polypeptide is capable of assembling into a recombinant AAV virion (rAAV); and wherein the rAAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8.

In some embodiments, the 581 to 589 region of the engineered VP capsid polypeptide, corresponding to residues 581 to 589, inclusive, with reference to SEQ ID NO: 1, has a sequence that is at least 70%, at least 75%, at least 77.7%, at least 80%, at least 85%, at least 88.8%,at least 90%, at least 91%, at least 92%, at least 93%, at least 94%, at least 95%, at least 96%, at least 97%, at least 98%, or at least 99% identical to a 581 to 589 region described herein. In some embodiments, the 581 to 589 region of the engineered VP capsid polypeptide comprises 1 amino acid substitution relative to a 581 to 589 region described herein. In some embodiments, the 581 to 589 region of the engineered VP capsid polypeptide comprises 2 amino acid substitutions relative to a 581 to 589 region described herein. In particular embodiments, the 581 to 589 region of the engineered VP capsid polypeptide has a sequence that is identical to a 581 to 589 region described herein.

In some embodiments, the engineered adeno-associated virus (AAV) viral protein (VP) capsid polypeptide is an engineered AAV5 viral capsid protein, wherein the engineered AAV VP5 capsid polypeptide has at least one substitution as compared to SEQ ID NO: 1 in the 581 to 589 region, corresponding to residues 581 to 589, inclusive, of SEQ ID NO: 1, inclusive; wherein the capsid polypeptide is capable of assembling into a recombinant AAV virion (rAAV); wherein the at least one substitution confers higher tropism for an ocular tissue on the rAAV as compared to an rAAV virion having an AAV5 VP capsid polypeptide of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8, optionally with further mutations elsewhere in the VP protein.

In some embodiments, the AAV VP capsid polypeptides have an amino acid sequence of SEQ ID NO: 2, wherein X¹, X², X³, X⁴, X⁵, X⁶, X⁷, X⁸, and X⁹ are each independently selected from A, R, N, D, C, E, Q, G, H, I, L, K, M, F, P, S, T, W, Y, and V; and wherein the polypeptide does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8.

In some embodiments, the engineered AAV VP capsid polypeptide comprises a polypeptide sequence represented by the formula: (A)-(X)-(B) wherein: (A) is the polypeptide sequence of SEQ ID NO: 12 (VAYNVGGQMATNNQSSTTAP, corresponding to residues 561 to 580 of SEQ ID NO: 2); (X) is a 581 to 589 region that confers ocular tissue tropism on a recombinant AAV virion (rAAV); and (B) is the polypeptide sequence of SEQ ID NO: 13 (IVPGSVWMERDVYLQGPIWA, corresponding to residues 590 to 609 of SEQ ID NO: 2); and wherein the capsid polypeptide is capable of assembling into the rAAV and, the capsid does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. Also encompassed herein are rAAVs composed of engineered AAV5 VP2 capsid polypeptides and engineered AAV5 VP3 capsid polypeptides comprising a 581 to 589 region that confers ocular tissue tropism at the region corresponding to amino acid residues 445 to 453 in AAV5 VP2 or the region corresponding to amino acid residues 389 to 397 in AAV5 VP3.

In some embodiments, the engineered AAV VP capsid polypeptide confers ocular tissue tropism or preference, wherein the ocular tissue is selected from the group consisting of aqueous humor, choroid, retina, optic nerve, vitreous humor, remaining eye tissue, and any combination thereof.

Described below are engineered mutated AAV5 VP1 polypeptide sequences that confer stable or improved virion assembly, tissue tropism, or both. In some embodiments, the present disclosure provides an AAV5 VP1 capsid polypeptide having a sequence homology of no more than 98.7% to SEQ ID NO: 1, wherein the AAV5 capsid polypeptide sequence has at least one mutation in a region from a position corresponding to 581 to a position corresponding to 589 of SEQ ID NO: 1. In some embodiments, an engineered AAV5 polypeptide comprises at least one amino acid substitution relative to residues 561 to 580 of SEQ ID NO: 1.

Also encompassed herein are rAAVs composed of engineered AAV5 VP2 capsid polypeptides and engineered AAV5 VP3 capsid polypeptides comprising a 581 to 589 region that confers ocular tissue tropism at the region corresponding to amino acid residues 445 to 453 in AAV5 VP2 or the region corresponding to amino acid residues 389 to 397 in AAV5 VP3.

Engineered VP Polypeptides Competent for rAAV Assembly

In various preferred embodiments, the mutated (engineered, recombinant) VP capsid polypeptides of the present disclosure are capable of forming an assembled virion, and in some instances that exhibit similar or improved stability when compared to a virion that comprises the AAV5 VP1 capsid polypeptide of SEQ ID NO: 1.

The frequency of a given amino acid residue occurring in assembled, purified viruses at a specified position within the 581 to 589 region, corresponding to position 581 to position 589 of SEQ ID NO: 1, or corresponding to X¹ to X⁹ as generalized in SEQ ID NO: 2, over the frequency of that given amino acid residue occurring at the specified position in the entire plasmid library was analyzed to identify sequence rules for capsid polypeptide sequences that favor viral capsid assembly. Based on the determined amino acid residue frequency, the contribution of each amino acid at each position of the 581 to 589 region to capsid assembly was determined, and sequences of the 581 to 589 region (X¹X²X³X⁴X⁵X⁶X⁷X⁸X⁹ of SEQ ID NO: 2) that favor capsid assembly, and rules for selecting sequences that favor capsid assembly, were identified.

Disclosed herein are engineered AAV5 VP capsid polypeptides capable of forming an assembled viral capsid that may exhibit similar or improved stability as compared to wild type AAV5 VP capsid polypeptide, wherein the engineered variant AAV5 VP capsid polypeptide sequence has one or more mutations, wherein the VP1 polypeptide sequence has said one or more mutations in a 581 to 589 region (corresponding to position 581 to position 589 in SEQ ID NO: 2), and wherein X¹ is selected from A, D, E, G, L, M, N, Q, S, T, or V, or X¹ is selected from A, D, E, M, or T. In some embodiments, X¹ is E; or X² is selected from A, C, D, E, G, H, I, N, P, Q, S, T, or V, or X² is selected from A, S, T, or V, or X² is A; or wherein X³ is selected from A, D, E, G, H, M, N, Q, S, T, or V, or X³ is selected from D, E, N, Q or T, or X³ is D or T; or wherein X⁴ is selected from A, D, E, G, H, N, P, Q, S, or T, or X⁴ is selected from D, E, P, or Q, or X⁴ is E; or wherein X⁵ is selected from A, C, D, E, G, H, N, Q, S, T, or Y, or X⁵ is selected from D, E, N, Q or T, or X⁵ is N; or wherein X⁶ is selected from A, D, E, G, H, N, P, Q, S, or T, or X⁶ is selected from D, N, or Q, or X⁶ is D; or wherein X⁷ is selected from A, C, D, E, G, H, N, Q, S, or T, or X⁷ is selected from A, D, E or G, or X⁷ is A; or wherein X⁸ is selected from A, C, D, E, G, H, N, Q, S, or T, or X⁸ comprises A, D, G, or S, or X⁸ is G; or wherein X⁹ is selected from A, D, E, G, H, N, P, Q, S, or T, or X⁹ is selected from A, D, G, or P, or X⁹ is G.

In various embodiments, the VP polypeptide is capable of forming an assembled viral capsid, and in some instances exhibits similar or improved stability when compared to a virion that comprises the AAV5 VP1 capsid polypeptide of SEQ ID NO:1.

Examples of amino acid substitutions within a 581 to 589 region that may favor viral capsid assembly are provided in TABLE 2. In some embodiments, the following amino acids can be independently mutated, in any combination, at any one or more positions X¹-X⁹, with reference to SEQ ID NO: 2, to provide an AAV VP1 capsid that is capable of assembling. Additionally, one or more mutations outside of the X¹-X⁹ region can be allowed, as long as the capsid is still capable of assembling.

TABLE 2
581 to 589 Region Amino Acid Variations for Viral Assembly
Viral Assembly Options

X¹ is A, D, E, G, L, M, N, Q, S, T, or V
X¹ is A, D, E, M, or T
X¹ is E
X² is A, C, D, E, G, H, I, N, P, Q, S, T, or V
X² is A, S, T, or V
X² is A
X³ is A, D, E, G, H, M, N, Q, S, T, or V
X³ is D, E, N, Q or T
X³ is D or T
X⁴ is A, D, E, G, H, N, P, Q, S, or T
X⁴ is D, E, P, or Q
X⁴ is E
X⁵ is A, C, D, E, G, H, N, Q, S, T, or Y
X⁵ is D, E, N, Q or T
X⁵ is N.
X⁶ is A, D, E, G, H, N, P, Q, S, or T;
X⁶ is D, N, or Q; or
X⁶ is D.
X⁷ is s A, C, D, E, G, H, N, Q, S, or T;
X⁷ is A, D, E or G; or
X⁷ is A.
X⁸ is A, C, D, E, G, H, N, Q, S, or T;
X⁸ is A, D, G, or S; or
X⁸ is G.
X⁹ is A, D, E, G, H, N, P, Q, S, or T;
X⁹ is A, D, G, or P; or
X⁹ is G.

### 581 to 589 Regions for Ocular Tissue Tropism

The present disclosure provides AAV5 virions with a VP capsid polypeptide having at least one mutation in a region with residues that interact with target cells (e.g., a target eye cell in a target ocular tissue of interest), where the at least one mutation confers increased ocular tissue tropism or preference as compared to a wild type VP capsid polypeptide (e.g., a wild type AAV5 capsid polypeptide of SEQ ID NO: 1). In some embodiments, provided herein are AAV5 VP1 capsid polypeptide having a sequence homology of at least 80% to SEQ ID NO: 1, wherein the AAV5 VP1 capsid polypeptide has at least one mutation in a 581 to 589 region, corresponding to residues 581 to 589 of SEQ ID NO: 1, and wherein said at least one mutation drives increased ocular tissue tropism as compared to the wild type VP capsid polypeptide (SEQ ID NO: 1). The following sequences rules and sequences also apply to the 581 to 589 region in AAV5 VP2 (amino acid residues 445 to 453; VP2 sequence shown in SEQ ID NO: 10) and in AAV5 VP3 (amino acid residues 389 to 397; VP3 sequences shown in SEQ ID NO: 11). Thus, the present disclosure encompasses AAV5 VP2 capsid polypeptides and AAV5 VP3 capsid polypeptides having one or more amino acid substitutions in the 581 to 589 regions of VP2 and VP3, corresponding to the AAV5 VP1 amino acid residues of the 581 to 589, where the one or more mutations comport to the rules or sequences in the following section.

VP capsid polypeptides (e.g., V1, VP2, or VP3 capsid polypeptides, or combinations thereof) comprising an ocular tissue-tropic 581 to 589 region, or any 581 to 589 region adhering to the rules identified herein) may assemble to form an rAAV with altered surface properties compared to a wild type AAV (e.g., comprising a VP1 polypeptide of SEQ ID NO: 1). The surface may form an interface that forms interactions with a target tissue, and the altered surface properties of the rAAV may promote tissue specific interactions (e.g., ocular tissue-specific interactions) between the rAAV and the target tissue. In some embodiments, the altered surface properties may be an altered charge distribution, increased or decreased hydrophobicity, altered availability or distribution of hydrogen bond donors or acceptors, or formation or reshaping of binding pockets. Such altered surface properties may favor interactions between the rAAV and ocular tissue while disfavoring interactions between the rAAV and non-ocular tissue. In some embodiments, the altered surface properties may favor interactions between the rAAV and a specific ocular tissue or ocular cells (e.g., retina, optic nerve, choroid, photoreceptor cells, aqueous humor, or vitreous humor) while disfavoring interactions between the rAAV and other ocular tissues or cells or non-ocular tissues.

An ocular tissue-tropic rAAV assembled from VP capsid polypeptides (e.g., V1, VP2, or VP3 capsid polypeptides, or combinations thereof) comprising a 581 to 589 region, or any 581 to 589 region adhering to the rules identified herein) may preferentially infect an ocular tissue (e.g., retinal tissue), ocular cells (e.g., retinal pigment epithelial cells, photoreceptor cells, rod photoreceptor cells, cone photoreceptor cells, or combinations thereof), or both, as compared to other ocular tissue (e.g., choroid, aqueous humor, or vitreous humor) or other ocular cells (e.g., photoreceptor cells) at a higher level as compared to wild type AAV5. The ocular tissue-tropic AAV may infect the ocular tissue at a rate that is at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.2-fold, at least about 2.4-fold, at least about 2.6-fold, at least about 2.8-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 100-fold, at least about 200-fold, at least about 300-fold, at least about 400-fold, or at least about 500-fold an infection rate for the other ocular tissue, as compared to wild type AAV5.

In some embodiments, the ocular tissue-tropic rAAV assembled from VP capsid polypeptides (e.g., V1, VP2, or VP3 capsid polypeptides, or combinations thereof) comprising a 581 to 589 region (e.g., any 581 to 589 region adhering to the rules identified herein) may deliver a payload to the tissue infected by the rAAV. The payload (e.g., a payload encoding a therapeutic peptide or a therapeutic polynucleotide) may be expressed in the tissue. In some embodiments, an expression level of the payload in an ocular tissue (e.g., retinal tissue), ocular cell (e.g., retinal pigment epithelial cell, photoreceptor cell, rod photoreceptor cell, cone photoreceptor cell, or combinations thereof), or both may have at least about 2.5-fold, at least about 2.8-fold, at least about 3-fold, at least about 3.2-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 20-fold, at least about 50-fold, at least about 75-fold, at least about 100-fold, at least about 200-fold, at least about 500-fold higher, or at least about 1000-fold an expression level in a different ocular tissue (e.g., choroid, vitreous humor, or aqueous humor) or ocular cell (e.g., photoreceptor cells). In some embodiments, the payload may be under control of a cell type- or tissue type-specific promoter to direct expression in a target cell type or target tissue type. For example, expression of the payload may be under control of a retinal-specific promoter to promote retinal-specific expression, a retinal pigment epithelium-specific promoter to promote retinal pigment epithelium-specific expression, a choroid-specific promoter to promote choroid-specific expression, a photoreceptor-specific promoter to promote photoreceptor-specific expression, or an optic nerve-specific promoter to promote optic nerve-specific expression.

In some embodiments, payload delivery to the ocular tissue using an ocular tissue-tropic AAV may have at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.2-fold, at least about 2.4-fold, at least about 2.6-fold, at least about 2.8-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 100-fold, at least about 200-fold, at least about 300-fold, at least about 400-fold, or at least about 500-fold higher specificity for the ocular tissue as compared to a wild type AAV (e.g., a wild type AAV5).

### Ocular Tissue-Tropic Sequences

Recombinant AAV viral capsids with specificity for ocular tissues (ocular tissue-tropic rAAVs) may be identified by screening rAAV libraries comprising VP capsid polypeptides with 581 to 589 regions, as described herein. The libraries may be screened in non-human primates (NHPs) by intravitreally administering the rAAV library and identifying sequences of 581 to 589 regions in VP capsid polypeptides that conferred tissue-tropic accumulation in or infection of ocular tissues (e.g., retina) to the rAAVs.

Disclosed herein are engineered AAV5 VP capsid polypeptides capable of forming an assembled virion that exhibits increased ocular tissue tropism or preference as compared to a wild type AAV VP capsid polypeptide (e.g., a VP1 of SEQ ID NO: 1, a VP2 of SEQ ID NO: 10, or aVP3 of SEQ ID NO: 11), wherein the engineered variant AAV5 VP capsid polypeptide sequence has one or more amino acid substitutions in a 581 to 589 region of the VP capsid polypeptide, corresponding to positions 581 to 589 in SEQ ID NO: 2 (X¹X²X³X⁴X⁵X⁶X⁷X⁸X⁹). In some embodiments, X¹ is selected from A, R, N, D, C, E, Q, G, H, I, L, K, M, F, P, S, T, W, Y, or V. In some embodiments, X² is selected from A, R, N, D, C, E, Q, G, H, I, L, K, M, F, P, S, T, W, Y, or V. In some embodiments, X³ is selected from A, R, N, D, C, E, Q, G, H, I, L, K, M, F, P, S, T, W, Y, or V. In some embodiments, X⁴ is selected from A, R, N, D, C, E, Q, G, H, I, L, K, M, F, P, S, T, W, Y, or V. In some embodiments, X⁵ is selected from A, R, N, D, C, E, Q, G, H, I, L, K, M, F, P, S, T, W, Y, or V. In some embodiments, X⁶ is selected from A, R, N, D, C, E, Q, G, H, I, L, K, M, F, P, S, T, W, Y, or V. In some embodiments, X⁷ is selected from A, R, N, D, C, E, Q, G, H, I, L, K, M, F, P, S, T, W, Y, or V. In some embodiments, X⁸ is selected A, R, N, D, C, E, Q, G, H, I, L, K, M, F, P, S, T, W, Y, or V. In some embodiments, X⁹ is selected from A, R, N, D, C, E, Q, G, H, I, L, K, M, F, P, S, T, W, Y, or V.

An ocular tissue-tropic 581 to 589 region may comprise one or more positively charged or basic amino acid residues. The positively charged or basic amino acid residues may be independently selected from K, R, or H. In some embodiments, at least one of X¹, X², X³, X⁴, X⁵, X⁶, X⁷, X⁸, or X⁹ of the 581 to 589 region is K, R, or H. In some embodiments at least two of X¹, X², X³, X⁴, X⁵, X⁶, X⁷, X⁸, or X⁹ of the 581 to 589 region are independently selected from K, R, or H. In some embodiments, at least three of X¹, X², X³, X⁴, X⁵, X⁶, X⁷, X⁸, or X⁹ of the 581 to 589 region are independently selected from K, R, or H.

An ocular tissue-tropic 581 to 589 region may comprise fewer negatively charged or acidic amino acid residues than the 581 to 589 region of a wild type VP polypeptide (e.g., SEQ ID NO: 1). The negatively charged or acidic amino acid residues may be D or E. In some embodiments, no more than one of X¹, X², X³, X⁴, X⁵, X⁶, X⁷, X⁸, or X⁹ of the 581 to 589 region is E or E. In some embodiments, none of X¹, X², X³, X⁴, X⁵, X⁶, X⁷, X⁸, or X⁹ of the 581 to 589 region are D or E.

In some embodiments, provided herein are AAV5 VP capsid polypeptide having at least one mutation in a 581 to 589 region, corresponding to residues 581 to 589 of AAV5 VP1 (e.g., SEQ ID NO: 1), wherein said at least one mutation drives increased ocular tissue tropism.

Retina Tissue-Tropic AAV VP Capsid Polypeptides. Described herein are engineered AAV VP capsid polypeptides comprising a variant 581 to 589 region predicted to confer increased retina tissue tropism, and predicted to confer increased retina tissue tropism compared to a wild type AAV VP capsid polypeptide (e.g., an AAV5 VP capsid polypeptide), based on a primary screen of a recombinant AAV5 capsid library. TABLE 3 provides 581 to 589 region sequences predicted to confer retina tissue tropism or preference.

TABLE 3
Exemplary 581 to 589 Region
Sequences for Retinal Targeting

| SEQ ID | |
|---|---|
| NO | 581 to 589 Region |
| 14 | AALHSRMYQ |
| 15 | AAMRWQRES |
| 16 | AAVLKYWQQ |
| 17 | ACCQKRRSQ |
| 18 | ACKGKQKWA |
| 19 | ACVSLRQGC |
| 20 | ADCPLYCQP |
| 21 | AEKGCRKYG |
| 22 | AFISTRRAG |
| 23 | AFKFKSDWG |
| 24 | AFLTIRRHY |
| 25 | AFMRPQQFP |
| 26 | AFVDNLMDP |
| 27 | AGALARKMM |
| 28 | AGVSKRTGS |
| 29 | AHKYSRTMQ |
| 30 | AHVKPVQTM |
| 31 | AHWPKQYDP |
| 32 | AICRKDRHQ |
| 33 | AIKATRFRN |
| 34 | AIKCREQMR |
| 35 | AIKELRLWP |
| 36 | AIKMSRRYE |
| 37 | AIKTRGCSY |
| 38 | AIRLAGPIG |
| 39 | AIYHDLRAM |
| 40 | ALKITRLME |
| 41 | ALKNVPLYG |
| 42 | ALKSRGKGD |
| 43 | ALKTTSKWP |
| 44 | AMCAKKKWC |
| 45 | AMKLHKSAG |
| 46 | AMKQKQAYQ |
| 47 | ANIQYRHGM |
| 48 | ANKASKRAC |
| 49 | APKPATKHP |
| 50 | AQYPMHRHQ |
| 51 | ARSRLKNQT |
| 52 | ASHGMGRQA |
| 53 | ASRQKYKCV |
| 54 | ATHMLGRHG |
| 55 | ATKQKTCLG |
| 56 | ATRGCCHSW |
| 57 | ATWKEELSE |
| 58 | AVAGWLRAE |
| 59 | AVGQSKHVP |
| 60 | AVHFKKCHL |
| 61 | AVKMKSTPP |
| 62 | AVRTSRHCF |
| 63 | AWKLKKSYM |
| 64 | AYKQKQKYN |
| 65 | CAKPKVAIC |
| 66 | CAKVHRNWD |
| 67 | CARFERHMY |
| 68 | CCKCRKYMS |
| 69 | CCQPKPWKW |
| 70 | CDYKRVKKY |
| 71 | CEMFWARVI |
| 72 | CFGTIRRGC |
| 73 | CFILLKARS |
| 74 | CFKEYRHHS |
| 75 | CIHPKSKYL |
| 76 | CIKQKKSSN |
| 77 | CIRSLFHAG |
| 78 | CKHTCNQCI |
| 79 | CKKHQGYMP |
| 80 | CKMPISKTD |
| 81 | CLNCKERRT |
| 82 | CMKIRQAMR |
| 83 | CMKTKGHMP |
| 84 | CMQRKCRMV |
| 85 | CQGAIRRHC |
| 86 | CQYHPRKWP |
| 87 | CRCKRTREQ |
| 88 | CSHGYSRVG |
| 89 | CSKMIRSLE |
| 90 | CSKTKNNVR |
| 91 | CSKVANVMH |
| 92 | CSRLRRWQF |
| 93 | CTFLYDEQY |
| 94 | CTKQKKRGN |
| 95 | CTRDGRKWG |
| 96 | CVLTSRMGS |
| 97 | CVRNVKNCM |
| 98 | DAFKYKRNA |
| 99 | DAHSYRTHF |
| 100 | DAKCRWKYC |
| 101 | DAKKLHKIE |
| 102 | DALVYRRNQ |
| 103 | DARINRTCY |
| 104 | DARQKKCYQ |
| 105 | DARQLRQYH |
| 106 | DARWRSITG |
| 107 | DAVRARYMQ |
| 108 | DCRQVRKVA |
| 109 | DFDGTFDFV |
| 110 | DFKSRHKTC |
| 111 | DFNRFRYHG |
| 112 | DGNKARVKN |
| 113 | DGRKYMRGC |
| 114 | DIARQRKFQ |
| 115 | DIIRMRRTT |
| 116 | DIKLKKKCD |
| 117 | DIKNIRSHH |
| 118 | DIRTSRVHV |
| 119 | DITKAKRAH |
| 120 | DKAKRGHSS |
| 121 | DKKDKKGWY |
| 122 | DKKRSQWSC |
| 123 | DKVRRERCH |
| 124 | DMKKRHGSC |
| 125 | DMRKAGQTY |
| 126 | DMSRQRAQF |
| 127 | DPKKKGTWS |
| 128 | DQAKFRRSY |
| 129 | DQYRMRQQQ |
| 130 | DRCKKQSGA |
| 131 | DRIGFKKQQ |
| 132 | DRKLKANWY |
| 133 | DSKYGRKGL |
| 134 | DSNKRTRYS |
| 135 | DSRHSRVME |
| 136 | DSRIMRSAE |
| 137 | DSRIVRTSS |
| 138 | DSRLIRCFH |
| 139 | DSSRKGQMN |
| 140 | DTKAWRNPS |
| 141 | DTKSRKEMD |
| 142 | DTKVKRHVA |
| 143 | DTKWSRMRA |
| 144 | DTRRCVHRS |
| 145 | DTRSKGRFT |
| 146 | DVKNRSMAW |
| 147 | DVKPSRGGY |
| 148 | DVKQRRAPH |
| 149 | DVKTRRIFS |
| 150 | DVNRKREYS |
| 151 | DVRAVRFWP |
| 152 | DVRRMAKAA |
| 153 | DVRSKALMC |
| 154 | DVRTERRIS |
| 155 | DVRTNRIEH |
| 156 | DVTSRKWLN |
| 157 | DWKPKRCMP |
| 158 | DYQPPRKSA |
| 159 | DYRQNDVAE |
| 160 | DYRRSLKAM |
| 161 | DYTRFRSHQ |
| 162 | EAGKGRKYW |
| 163 | EAKAKQMPG |
| 164 | EAKGRVIGP |
| 165 | EAKGTRCPF |
| 166 | EAKIRNFTM |
| 167 | EAKMFVRHH |
| 168 | EAKNIRCKA |
| 169 | EAKTKRFCR |
| 170 | EAKVQRGYN |
| 171 | EAKWSGRKN |
| 172 | EAMWWRHGG |
| 173 | EARLTRCCL |
| 174 | EARSCRCGL |
| 175 | EARTRAHIG |
| 176 | EARVIRSNV |
| 177 | EARYKGTQY |
| 178 | EASIKKQAM |
| 179 | EASKRMIVK |
| 180 | ECHKIKKGY |
| 181 | ECRFNRKKG |
| 182 | EERPRIITR |
| 183 | EFHRKTRAS |
| 184 | EFKPRKSGA |
| 185 | EFKRKHIQQ |
| 186 | EFRAQRTLA |
| 187 | EFRKSGSCY |
| 188 | EFRMVRAQE |
| 189 | EFRYQRAHC |
| 190 | EFSSRWSVF |
| 191 | EGKPRSRLG |
| 192 | EGKSRRHHD |
| 193 | EGKYGKKHG |
| 194 | EGRGMRRQC |
| 195 | EHKQKMRER |
| 196 | EHRPVRSQM |
| 197 | EHRTRHGWT |
| 198 | EHVRLKHLA |
| 199 | EIAKYRWVQ |
| 200 | EIKPCRHRC |
| 201 | EIKPRRIAS |
| 202 | EIKSKKKFD |
| 203 | EIKWQRGYY |
| 204 | EIRKSGHKQ |
| 205 | EIRMNKSNA |
| 206 | EIRTSKEAR |
| 207 | EIWKCRRQV |
| 208 | EKAIMKLRS |
| 209 | EKAPSKKGI |
| 210 | EKGYNKHRG |
| 211 | EKKLLNRWW |
| 212 | EKKPKNRYN |
| 213 | EKKTKQRMQ |
| 214 | EKKWRGIMP |
| 215 | EKREKHRQT |
| 216 | EKVRTCWRG |
| 217 | ELKGKKAIQ |
| 218 | ELRRYGFQH |
| 219 | EMKAKKCHT |
| 220 | EMKLMKKVW |
| 221 | EMKMGRQWA |
| 222 | EMKQMRKFP |
| 223 | EMRVKRTER |
| 224 | ENKSAKRGC |
| 225 | ENTRRKFTS |
| 226 | EPKLKVYKS |
| 227 | EPKSRPFKC |
| 228 | EQARSRKVG |
| 229 | EQGRTKKMY |
| 230 | EQKLRKAMP |
| 231 | EQKNSRAQF |
| 232 | EQRQFRAKD |
| 233 | EQRSSRYAA |
| 234 | ERCRFKNGN |
| 235 | ERHTMHKAP |
| 236 | ERKGTVKMT |
| 237 | ERKQKGTCG |
| 238 | ERVQARKYC |
| 239 | ESFKRKVGH |
| 240 | ESKSYKCLN |
| 241 | ESMRGRKMP |
| 242 | ESRDVRAMN |
| 243 | ESRFNRSQG |
| 244 | ESRFRKDSN |
| 245 | ESRIKREWH |
| 246 | ESRKAHHDR |
| 247 | ESRPFRRFH |
| 248 | ETARKGRWY |
| 249 | ETKKHSSFW |
| 250 | ETKLFKWQD |
| 251 | ETKLKKADK |
| 252 | ETKRHQMAQ |
| 253 | ETKRRQCGY |
| 254 | ETKVHKVIG |
| 255 | ETKYRKRAE |
| 256 | ETRAGRTKG |
| 257 | ETRHRMVGC |
| 258 | ETRLGKHTY |
| 259 | ETRLMRSVD |
| 260 | ETRPQRMGH |
| 261 | ETRRIQRWD |
| 262 | ETRYRANIG |
| 263 | ETVRQKHNQ |
| 264 | EVKIKKRTI |
| 265 | EVKNKGRGQ |
| 266 | EVKPARSTF |
| 267 | EVKSRQCWQ |
| 268 | EVLRMRHEQ |
| 269 | EVNRNKMRT |
| 270 | EVQRSKRIS |
| 271 | EVRPFRQGQ |
| 272 | EVRSSRLQN |
| 273 | EWKKRTTWN |
| 274 | EYARFKGHQ |
| 275 | EYKHKKTGW |
| 276 | EYKLQRSAQ |
| 277 | FAKDRRNPG |
| 278 | FAKEMRVYE |
| 279 | FAKMSGRYA |
| 280 | FAMDIRFAQ |
| 281 | FAVARSQQC |
| 282 | FMKNKRNYG |
| 283 | FPRNVTMLS |
| 284 | FQAPSRISP |
| 285 | FRDCKIRRY |
| 286 | FSQATRRQV |
| 287 | FTKAWAKRE |
| 288 | FVWSYMFGP |
| 289 | GAFCRLSAD |
| 290 | GAIRSTHSC |
| 291 | GAKTFQKLQ |
| 292 | GERLGRKVP |
| 293 | GFRMKEKMS |
| 294 | GFVKTNKSA |
| 295 | GFWPYLYMG |
| 296 | GIGQKKSWY |
| 297 | GIKSHKREP |
| 298 | GIVRRNTAN |
| 299 | GKGTMCCEC |
| 300 | GKNRCKFQC |
| 301 | GLGTKQFRN |
| 302 | GMAVKRREP |
| 303 | GMFKLNQSC |
| 304 | GMKVHRKAP |
| 305 | GMLQNRSHQ |
| 306 | GNKATNIML |
| 307 | GQGLFRRQC |
| 308 | GQKGSGRQA |
| 309 | GQKLRTSSG |
| 310 | GRSQKDWGI |
| 311 | GSAWQIWSD |
| 312 | GSKFTRQCY |
| 313 | GSKQQKGGV |
| 314 | GSKQTTGYT |
| 315 | GSKSHSHWM |
| 316 | GSKSNYHSG |
| 317 | GSSSKTTCC |
| 318 | GTAGCKKAA |
| 319 | GTAMFRVSA |
| 320 | GTIKRMTAY |
| 321 | GTKVKNCRN |
| 322 | GTKWSKKWQ |
| 323 | GVFDIRKNP |
| 324 | GVHWKKTAY |
| 325 | GVKFRNKTN |
| 326 | GVKLHTPFS |
| 327 | GVTGRFCCQ |
| 328 | GVVLIRQDD |
| 329 | GYKQQRNRD |
| 330 | GYKQTHWLG |
| 331 | GYWWLFADR |
| 332 | HASRKGTFP |
| 333 | HAWPMPATW |
| 334 | HKQWNYCPS |
| 335 | HKRTAPWTN |
| 336 | HQKYKKLTG |
| 337 | HRRPFQTKT |
| 338 | HVRSNLPIC |
| 339 | IAFQYQNRL |
| 340 | IAKMKSRYP |
| 341 | IARSGWMSG |
| 342 | IAYHYWKDV |
| 343 | ICLKRHAKA |
| 344 | IEKQRRVYA |
| 345 | IGAKRSWTS |
| 346 | IGIPKNHKP |
| 347 | IGRWMINSM |
| 348 | IGVKAHRSA |
| 349 | IHHTNFHSP |
| 350 | IHMKRAAYS |
| 351 | IIKPRSKVD |
| 352 | IKQKGELKC |
| 353 | ILLKHMKYP |
| 354 | IMKDRKSGS |
| 355 | IMPRCWDMC |
| 356 | IPQPSRKMP |
| 357 | IPRTINRCT |
| 358 | IQHRFLTYQ |
| 359 | IQKCMTRQA |
| 360 | ISTQPRQQS |
| 361 | ISWIQRTCM |
| 362 | ITFGTRKDA |
| 363 | ITFWKMRDG |
| 364 | ITKDIRWKG |
| 365 | ITRDSRWGM |
| 366 | ITTTARKVP |
| 367 | IVKEVRTNH |
| 368 | IVKPRGQCI |
| 369 | IVKQLKRTA |
| 370 | IVKYRVGTS |
| 371 | IVVESENRH |
| 372 | IVVRRFYDP |
| 373 | IVYSSRQNP |
| 374 | KAGTPGIQN |
| 375 | KEARHTRRE |
| 376 | KEFLYLSRE |
| 377 | KEKEYKKYF |
| 378 | KFRDAQNMS |
| 379 | KIKVANAAS |
| 380 | KKPANMFPW |
| 381 | KNRAVQGFN |
| 382 | KPGMVMNPN |
| 383 | KPRPKLNST |
| 384 | KQRHWGAKW |
| 385 | KQRSGWCCH |
| 386 | KSCKGRANG |
| 387 | KSFYGEHYF |
| 388 | KSNQMGKSP |
| 389 | KVKVRACSD |
| 390 | LAKDCRACN |
| 391 | LAKFKNKIL |
| 392 | LANTYHWEQ |
| 393 | LANVKGRVA |
| 394 | LAPRKQVQW |
| 395 | LARAKTSSV |
| 396 | LARYLGSQN |
| 397 | LCKEGRKAG |
| 398 | LCKWSKKQC |
| 399 | LFRMNMRTH |
| 400 | LGKSRKQMA |
| 401 | LIECRRRCP |
| 402 | LIKCKDRFT |
| 403 | LIKWNRKAM |
| 404 | LIRLYTNKE |
| 405 | LLCKVKDYQ |
| 406 | LLIKKNRGV |
| 407 | LLRLAQQGP |
| 408 | LLRMTTYAW |
| 409 | LMKICKSFP |
| 410 | LMWSRFEVP |
| 411 | LNMHYRSHK |
| 412 | LNRQRGHAS |
| 413 | LPERCRKQS |
| 414 | LPKPTKKGP |
| 415 | LPKSTKRWG |
| 416 | LQKMSRFMN |
| 417 | LQKTRSIIQ |
| 418 | LQTKKNRHY |
| 419 | LRVPRYGQS |
| 420 | LSKKRERCN |
| 421 | LSKTKSYVE |
| 422 | LTRFHGGMY |
| 423 | LVKDRCSGY |
| 424 | LVRMHRSDQ |
| 425 | LVSRLPSMR |
| 426 | LVVQRNVRQ |
| 427 | LWKTSRQCN |
| 428 | LYIHRWVSG |
| 429 | MAEHQIVTT |
| 430 | MAIRGTKAN |
| 431 | MAKLCQRKC |
| 432 | MALDFRNGK |
| 433 | MCAGYHREQ |
| 434 | MCKAHMRKD |
| 435 | MCTATSRYK |
| 436 | MEKQLKLHN |
| 437 | MFVTYYTLY |
| 438 | MGAPKKTLG |
| 439 | MHWSVHYNS |
| 440 | MHYLAMSAR |
| 441 | MIAEDEYWA |
| 442 | MIDSRKRSA |
| 443 | MISLKKKMS |
| 444 | MKILKYSQE |
| 445 | MKKDKTNST |
| 446 | MKLNRNHDC |
| 447 | MKSRPFMGS |
| 448 | MMAGMRISS |
| 449 | MMAKPLWAY |
| 450 | MMHQVTHDF |
| 451 | MMKGRSYCT |
| 452 | MMKPKGHYP |
| 453 | MNIKKNHWA |
| 454 | MPKRACNKW |
| 455 | MPWKIDTGG |
| 456 | MQKTSKKYQ |
| 457 | MQQRVFRSN |
| 458 | MQSLKSRWH |
| 459 | MSFKDLKNG |
| 460 | MSGSRRNAE |
| 461 | MSKQKKAGS |
| 462 | MSKSRSLNN |
| 463 | MSPFVRKAS |
| 464 | MSRESRSVS |
| 465 | MSWLERKAN |
| 466 | MTEGKKRIP |
| 467 | MTGKSPRGA |
| 468 | MTGVRWRSQ |
| 469 | MTIYQRGAA |
| 470 | MTMCLKKYY |
| 471 | MTMTMLPWY |
| 472 | MTRGHSIWG |
| 473 | MTRMRLSGP |
| 474 | MTTSRQRCW |
| 475 | MTWYHDIHQ |
| 476 | MVKHHRESF |
| 477 | MVKWSWHKC |
| 478 | MVMKRVMVP |
| 479 | MVVPAKYKW |
| 480 | MVWSADSYE |
| 481 | MWKDIRQWH |
| 482 | MWKSRLDSY |
| 483 | MYKCGIKCH |
| 484 | MYRAKLTAQ |
| 485 | NAMWLRHYD |
| 486 | NCAQYYGTG |
| 487 | NFKDMRSQE |
| 488 | NIGLKKKYF |
| 489 | NIRQPNQCA |
| 490 | NKHSQRREA |
| 491 | NKRDHKYWG |
| 492 | NKTKSHYGN |
| 493 | NMNHVAFAY |
| 494 | NMYMYRAQH |
| 495 | NNKGLSQNT |
| 496 | NQRLYAKSE |
| 497 | NRMRYSCYS |
| 498 | NSKTWGTLS |
| 499 | NTFISYRDQ |
| 500 | NTGKRSRQA |
| 501 | NTHALGRHG |
| 502 | NTKLSRYGM |
| 503 | NTKSAVKQP |
| 504 | NTLPSRYGN |
| 505 | NTNFRNMSW |
| 506 | NTNPSGKNF |
| 507 | NTYCKKHCS |
| 508 | NVAPKSHKW |
| 509 | NVAWRTRDL |
| 510 | NVKGIAHYC |
| 511 | NVKTLHHMW |
| 512 | NVSMLGRWN |
| 513 | NVTNQKKTW |
| 514 | NWNPCRKNA |
| 515 | NYWWMLSGP |
| 516 | PCGGKVIKA |
| 517 | PKRKGNEWC |
| 518 | PMKPTKYAH |
| 519 | PSSVKQTKA |
| 520 | PTKVLNRYN |
| 521 | PWAGTRWKQ |
| 522 | QAGCKGTWR |
| 523 | QAHMFFKHE |
| 524 | QAKWRNLWN |
| 525 | QAMHYWRYP |
| 526 | QARDRTAQF |
| 527 | QAVARGNLK |
| 528 | QCCKRKTGL |
| 529 | QCKLKGNLM |
| 530 | QCKTKKSAS |
| 531 | QCKYRYTKG |
| 532 | QCQPRKHTP |
| 533 | QCVCKKMKG |
| 534 | QCWPTMKCD |
| 535 | QDYQEDGKK |
| 536 | QEKKKANLT |
| 537 | QFKLKTRES |
| 538 | QGAGGRKDQ |
| 539 | QGALYKKQF |
| 540 | QIAKVQKTR |
| 541 | QIFRLPQAG |
| 542 | QIRDKSRHH |
| 543 | QLTRYMKST |
| 544 | QMHHKKNWM |
| 545 | QMHPESYSA |
| 546 | QMKSKTKWL |
| 547 | QMWFRYASE |
| 548 | QNSQIKKVG |
| 549 | QPEQRRKVP |
| 550 | QPKLRSQVM |
| 551 | QPKTRSMYQ |
| 552 | QPKVNRHTG |
| 553 | QPLYFRSQG |
| 554 | QQMTKKNFG |
| 555 | QSAGRKTVA |
| 556 | QSFLSLTPG |
| 557 | QSHMYGRIS |
| 558 | QSIYFRHGI |
| 559 | QSQNLRRQC |
| 560 | QSYWNLSYQ |
| 561 | QTKTYREGG |
| 562 | QTRGGCKRA |
| 563 | QTTQPRQWP |
| 564 | QVFATLTSW |
| 565 | QVFLNLREG |
| 566 | QVHGILRAH |
| 567 | QVKWKNRFP |
| 568 | QVSKCQIRK |
| 569 | QVSMRGHYR |
| 570 | QVVRNHHKE |
| 571 | RCGYHHSQT |
| 572 | RCRVAIVWT |
| 573 | RFAYWKNQG |
| 574 | RHQKRNWAN |
| 575 | RMFSVPMWD |
| 576 | RNKPIKHGP |
| 577 | RPNRVMARM |
| 578 | RTCYWSANE |
| 579 | RVHPWSQRQ |
| 580 | RWKDKNKLM |
| 581 | RWKHLKGVC |
| 582 | SAGWRSHCV |
| 583 | SALYHGAGF |
| 584 | SASWKGKTP |
| 585 | SCKPKSKST |
| 586 | SCMRKGRFQ |
| 587 | SDVRKPRQH |
| 588 | SECNNPFVM |
| 589 | SGCQMKRAF |
| 590 | SHKCKTSMT |
| 591 | SHKEIKGQA |
| 592 | SHPNTRQGD |
| 593 | SIGMRSHMG |
| 594 | SIKVQKRAG |
| 595 | SIQRLKWVN |
| 596 | SKDSFRRCC |
| 597 | SKHPRMQQA |
| 598 | SKHQYHQDA |
| 599 | SKKTGGIQT |
| 600 | SLKQQSYRG |
| 601 | SLRQNHMHQ |
| 602 | SNTTVRKQY |
| 603 | SQLSFRSAT |
| 604 | SQPRNKFSG |
| 605 | SRHRYPKCS |
| 606 | SRRVSSRMF |
| 607 | SRVKMSRQH |
| 608 | SSMPMFSGA |
| 609 | STAQKCKFM |
| 610 | STFYRFRDP |
| 611 | STGPKKNST |
| 612 | STKISRLHN |
| 613 | STKSRSHYH |
| 614 | STSSAKKYP |
| 615 | SVGYGKPWQ |
| 616 | SVHMNLRAH |
| 617 | SVKLRSWSK |
| 618 | SVKPCCRNR |
| 619 | SVRDIRYGP |
| 620 | SWWPLMTWG |
| 621 | SYVKPIQRP |
| 622 | SYWGYSRAH |
| 623 | TAKYRGLWQ |
| 624 | TARQFKSPR |
| 625 | TASSRLCCH |
| 626 | TCKGVSYRT |
| 627 | TCKPHGKCT |
| 628 | TCKYRSHLA |
| 629 | TCRGYNDSY |
| 630 | TFTRPLCMS |
| 631 | TGAFRRSGA |
| 632 | TGKLRGGWH |
| 633 | TGRFSSHMH |
| 634 | TGVKRNHAG |
| 635 | THHKARYTG |
| 636 | THKEKRTAI |
| 637 | THSSFKKMQ |
| 638 | TIIRRNTTT |
| 639 | TIIWARSMA |
| 640 | TIKTSRTDD |
| 641 | TIKYKSGNG |
| 642 | TIRDLRTQN |
| 643 | TMAQSWRSI |
| 644 | TMKSKQRWV |
| 645 | TMRSKSCSM |
| 646 | TMRWRNHSE |
| 647 | TNKFTRKGW |
| 648 | TNWLIKWMG |
| 649 | TNWPHVKAY |
| 650 | TPKSRGVYA |
| 651 | TPKVKSSFN |
| 652 | TPRPSTRAP |
| 653 | TQCQKKTAR |
| 654 | TQFQKLADR |
| 655 | TQKPKKSGS |
| 656 | TQNASRHMQ |
| 657 | TQPKLKRES |
| 658 | TQRDRMRAN |
| 659 | TQRPTTKTR |
| 660 | TSAFKKGFT |
| 661 | TSHAIIRLQ |
| 662 | TSKFFGMFA |
| 663 | TSKMPGRAS |
| 664 | TSKPNRKKT |
| 665 | TSSYKSRFH |
| 666 | TSWWLPSMT |
| 667 | TTARNQGTG |
| 668 | TTKGQVQCM |
| 669 | TTKGTGSHN |
| 670 | TTKPGRHSY |
| 671 | TTKTVREYG |
| 672 | TTRYSTAPS |
| 673 | TTVKRLNSF |
| 674 | TTWTPEQHP |
| 675 | TVKNNVLCG |
| 676 | TVKPQMVGR |
| 677 | TVKWGNKLW |
| 678 | TVNIKTRSH |
| 679 | TVREKGHDR |
| 680 | TVSSRQRTR |
| 681 | TWKTRTCAP |
| 682 | TWRYVMKEC |
| 683 | TYAKYNRMC |
| 684 | TYGFPVSVA |
| 685 | TYKYKNNYC |
| 686 | VAALNLRHP |
| 687 | VACKCKNRP |
| 688 | VAHNALKMM |
| 689 | VAKVMTTAP |
| 690 | VAKWRSKHQ |
| 691 | VARIKQDMF |
| 692 | VASRGGRGC |
| 693 | VASSQKRAI |
| 694 | VCKVNRKML |
| 695 | VCRHRQQMY |
| 696 | VCVKKYRCN |
| 697 | VGAGYKHCY |
| 698 | VHRFVNRDF |
| 699 | VIKGQATER |
| 700 | VIKSSWFKG |
| 701 | VIMRPISQQ |
| 702 | VKHKDQWGP |
| 703 | VKHRAKFCD |
| 704 | VKQFRKCFC |
| 705 | VLDRHWAAY |
| 706 | VLKQIGRNS |
| 707 | VMKDVKKVW |
| 708 | VMKDWKGKH |
| 709 | VMMLARQSQ |
| 710 | VNPYKLWKT |
| 711 | VPFNYWRME |
| 712 | VPIRVMRHG |
| 713 | VPIYTKWKN |
| 714 | VQKTSRRGE |
| 715 | VQVRRHAHG |
| 716 | VSDIANEAF |
| 717 | VSKAKSNFR |
| 718 | VSPTIRKSP |
| 719 | VSSLKKSQF |
| 720 | VSTRDKWRN |
| 721 | VTCRCKMKH |
| 722 | VTDRKQLRG |
| 723 | VTKQKCKMN |
| 724 | VTQMKHKGW |
| 725 | VTRQKCKMN |
| 726 | VVGKVMRDC |
| 727 | VVKQVGKQA |
| 728 | VVNYTALQD |
| 729 | VWGLTRHAG |
| 730 | WAKPTKKIM |
| 731 | WEPGIITGC |
| 732 | WVFSIQKQA |
| 733 | YIATKLRDQ |
| 734 | YIRDRGHYP |
| 735 | YIYPITYSQ |
| 736 | YMFEAPWIE |
| 737 | YSGAKGHMM |
| 738 | AAGASKNAK |
| 739 | AAHRPMFTS |
| 740 | AAKSFRRAA |
| 741 | AARSTQGIA |
| 742 | AATHKYRCN |
| 743 | AATKEKRHM |
| 744 | AAYTSRQLG |
| 745 | ACKIKTGQA |
| 746 | ACTKERSIR |
| 747 | ADKTKKHFA |
| 748 | AFKTMRWSM |
| 749 | AFPPYKYRK |
| 750 | AHRSKKTSM |
| 751 | AIYSAMTAF |
| 752 | AKAKMSRTY |
| 753 | AKKASKRGC |
| 754 | AKPIPQTKN |
| 755 | ALRLKNSLT |
| 756 | AMKMKKTHF |
| 757 | AMSKAIRNS |
| 758 | ANRFKNTEG |
| 759 | AQKSLHYTC |
| 760 | ASKPRKRYN |
| 761 | ATFRDSDYQ |
| 762 | ATHERRKSP |
| 763 | ATTKRKGCC |
| 764 | ATTRFPDAA |
| 765 | AVRFGAAQK |
| 766 | AVRLRSKVD |
| 767 | AWIRCIQMP |
| 768 | AYKQVKREL |
| 769 | AYMVRLNHH |
| 770 | AYMYRPATE |
| 771 | CAKGKTQTS |
| 772 | CASRKQRSH |
| 773 | CGSSYKKVQ |
| 774 | CIVQKTAQK |
| 775 | CKCGLKRFS |
| 776 | CKCLKTMKQ |
| 777 | CKTRACKYC |
| 778 | CRCKCAIKC |
| 779 | CSKYTAKAW |
| 780 | CSQGTKKSY |
| 781 | CSTQGRKAM |
| 782 | CSTSKKRTF |
| 783 | CSTSQRRWG |
| 784 | CTGPCCRPQ |
| 785 | CTKHLARQH |
| 786 | CTKKTRCQG |
| 787 | CVDILLVHR |
| 788 | CVGLDRRGY |
| 789 | DAKFSRGMQ |
| 790 | DAKFVRHQH |
| 791 | DAKPSRCCL |
| 792 | DAKSKKKWY |
| 793 | DANIEWHGN |
| 794 | DARKWSNCI |
| 795 | DARMKGISS |
| 796 | DASMWSGIE |
| 797 | DDGKEVWHH |
| 798 | DGFPKRKAC |
| 799 | DGSSIRRYP |
| 800 | DHFHPRQSP |
| 801 | DIARGKGKP |
| 802 | DIKMHRGAC |
| 803 | DKKDRKMIY |
| 804 | DKKGYALAY |
| 805 | DKKQRQWTA |
| 806 | DMQRKKREG |
| 807 | DQKFMKTYF |
| 808 | DRLPSRQCQ |
| 809 | DRRGKTSMP |
| 810 | DRRGYGVSN |
| 811 | DSQKPKKAP |
| 812 | DSRCFRRGY |
| 813 | DSRNRAHWC |
| 814 | DSSRARYKS |
| 815 | DTKQKGSPK |
| 816 | DTSRVKAAQ |
| 817 | DVKLKKRYY |
| 818 | DVKPRRSGF |
| 819 | DVRTSRGVT |
| 820 | DVRWIRHHH |
| 821 | DYHRLGRQE |
| 822 | EAKLAKSYS |
| 823 | EAKYKKNYS |
| 824 | EARGRAHSG |
| 825 | EARTSRWLA |
| 826 | ECKPKAKWM |
| 827 | ECKPRRMQA |
| 828 | ECRPTRINE |
| 829 | EFKHRWQGR |
| 830 | EGKLTKRCM |
| 831 | EIFKALRAE |
| 832 | EIRPCRFRN |
| 833 | EIRRQGQAI |
| 834 | EIRVIKSHS |
| 835 | EKHTFKKWA |
| 836 | EKKEAKKFN |
| 837 | EKKHKGEFW |
| 838 | EKRSISVHT |
| 839 | EMKVTKMRG |
| 840 | ENKLVKKTP |
| 841 | EQQCFLHGN |
| 842 | EQRIMRSQA |
| 843 | ERFHKMSAD |
| 844 | ERKARSTYC |
| 845 | ERQQRCRFS |
| 846 | ERVPMWRNA |
| 847 | ERYPQRSAF |
| 848 | ESGCNQPQD |
| 849 | ESGRKSRAE |
| 850 | ESIRAKRVA |
| 851 | ESKGYRSGC |
| 852 | ESKIKSAAS |
| 853 | ESKNVKDWK |
| 854 | ESKYRNRMC |
| 855 | ETHRKNQHN |
| 856 | ETIPFLDLG |
| 857 | ETKYVRKEW |
| 858 | ETRMRANFQ |
| 859 | ETRPFRGNE |
| 860 | ETRSRCHCA |
| 861 | ETRVSRNMP |
| 862 | ETRYFKNAY |
| 863 | ETVERCNFI |
| 864 | ETVRQKGHN |
| 865 | EVGNRKWQS |
| 866 | EVKRAPSTL |
| 867 | EVKWRKCLQ |
| 868 | EVLRPRAIA |
| 869 | EVRALRTQH |
| 870 | EVRMSRTIN |
| 871 | EVRQSRQVC |
| 872 | EVRSARQQS |
| 873 | EVTRHRLWS |
| 874 | EWHRFGRNS |
| 875 | EWWPIRHSW |
| 876 | EYKPCRRAV |
| 877 | EYKTARHAM |
| 878 | FAYKEWLPG |
| 879 | FFHSRMSQE |
| 880 | FHRALFHAG |
| 881 | FICMKKTDQ |
| 882 | FIGWMHRAN |
| 883 | FIWPIDHTS |
| 884 | FLNEYGYYQ |
| 885 | FSFQYLKKG |
| 886 | FTATSLRFP |
| 887 | GAGMKKRMS |
| 888 | GAKLKSRLD |
| 889 | GDKSRKSKM |
| 890 | GFSMMMIPW |
| 891 | GHKETRAMG |
| 892 | GILRKVNDH |
| 893 | GNVKIMRFA |
| 894 | GQKTTSTNY |
| 895 | GSKMYAHTP |
| 896 | GSRDTRQYP |
| 897 | GTHLELMNA |
| 898 | GTKQKAVIE |
| 899 | GTREHGKTP |
| 900 | GTWSAMRAT |
| 901 | GVKCQKSAK |
| 902 | GVKGHSHDR |
| 903 | GVWPCYYLQ |
| 904 | GYMECDHMQ |
| 905 | GYRFVGTTP |
| 906 | HAMIFQRAS |
| 907 | HAPMYKSAG |
| 908 | HNKDLKCQE |
| 909 | HRRMRDAIY |
| 910 | HSQRCKVIA |
| 911 | IAKGQQQMC |
| 912 | IAKQKCNCC |
| 913 | IAMRKWNCE |
| 914 | IANQLTMTT |
| 915 | IASILWSSA |
| 916 | IAVRYYHQW |
| 917 | ICKPKGKWL |
| 918 | IERKNNASQ |
| 919 | IFRLWNAKE |
| 920 | IGGPYRTIN |
| 921 | IGKYTKKMT |
| 922 | IGRFNNIAE |
| 923 | IIRPSSHAC |
| 924 | IIRSGTKYP |
| 925 | IMRGGTKYG |
| 926 | INKTVQRCG |
| 927 | INRPTRWPH |
| 928 | INVRSQHQH |
| 929 | ISNISKWRT |
| 930 | ISQSMKFSG |
| 931 | ISRDQKHVC |
| 932 | ITFPRIRAD |
| 933 | KAHAITDHS |
| 934 | KGMRCKAME |
| 935 | KHLMVPGKM |
| 936 | KKMGRMCDR |
| 937 | KMHRCKDDR |
| 938 | KNEVDTNGE |
| 939 | KSAVHQGGH |
| 940 | KSDTFRRVF |
| 941 | KTHKFSSCL |
| 942 | KVRSNREQM |
| 943 | KWQGSTHKT |
| 944 | KWTYNNTDL |
| 945 | LAGKMAHWQ |
| 946 | LAYMKILQQ |
| 947 | LCNIRCRSG |
| 948 | LDAQIVCYG |
| 949 | LFKAAVKAP |
| 950 | LFKPKKTSQ |
| 951 | LGKFSGAGS |
| 952 | LHKQYYHGM |
| 953 | LHWLIKESG |
| 954 | LKERKEVRA |
| 955 | LKGSSKSSF |
| 956 | LLWINTMIW |
| 957 | LMFYTLSRD |
| 958 | LMRNVNVEP |
| 959 | LNRQHGHAP |
| 960 | LNRYVSMGV |
| 961 | LPKVARKMF |
| 962 | LQGRYMGAQ |
| 963 | LQKNGRYSG |
| 964 | LSIRRNCCG |
| 965 | LSRHTKQLA |
| 966 | LSRPTMTIS |
| 967 | LTAKPFKCQ |
| 968 | LTGKYVGYS |
| 969 | LTKSRGQCS |
| 970 | LTRAHRSHE |
| 971 | LVIGRHQNW |
| 972 | LVRDIREFS |
| 973 | LVRYKSGGD |
| 974 | LVYTSLKDA |
| 975 | MAKQKKQAM |
| 976 | MAMEQRRFG |
| 977 | MAMREMKVC |
| 978 | MAMYYRMDD |
| 979 | MCRDRGKMV |
| 980 | MDGRFKATS |
| 981 | MEGCFMYVF |
| 982 | MGWPKHQDP |
| 983 | MHKVKSGNA |
| 984 | MIKNSGKEL |
| 985 | MIRGQRGGG |
| 986 | MIWPMSVQH |
| 987 | MLKMGFRYA |
| 988 | MLKYRQGGG |
| 989 | MLTYWTSRN |
| 990 | MMKCKRWDY |
| 991 | MMKSHKAGN |
| 992 | MNKDKRIMP |
| 993 | MNVQKRNYF |
| 994 | MPKLNRKAQ |
| 995 | MPKNYGHAP |
| 996 | MPMIKLSYS |
| 997 | MQAQPRKQG |
| 998 | MQHYLHHAT |
| 999 | MQRYKSKCC |
| 1000 | MQYMQRNAY |
| 1001 | MSKESRSQP |
| 1002 | MSLGQRTSW |
| 1003 | MSMVRKDQQ |
| 1004 | MTAGKKAWN |
| 1005 | MTTLQRQFM |
| 1006 | MVIPVEIMI |
| 1007 | MVKMKCTCT |
| 1008 | MVWPITRTD |
| 1009 | MVWPKQTEA |
| 1010 | MVWWEEKMC |
| 1011 | MVYRPLTSS |
| 1012 | MWAGKSRWC |
| 1013 | MWAPFRHWN |
| 1014 | MWAQTRQQW |
| 1015 | MYWHQTLSE |
| 1016 | NAKLNKRQM |
| 1017 | NHKSYPVAP |
| 1018 | NKHSTRAMD |
| 1019 | NRHKCGSWS |
| 1020 | NSGLKKYYG |
| 1021 | NSRNMLQNE |
| 1022 | NTKIRSVQH |
| 1023 | NTKSKSQLA |
| 1024 | NTVRTFHGW |
| 1025 | NVHAKLSAE |
| 1026 | NVKPTAWKW |
| 1027 | NYRSHRKIA |
| 1028 | PAVKKVERQ |
| 1029 | PCKLAGTWD |
| 1030 | PIGKHAFKC |
| 1031 | PLVKSGFGT |
| 1032 | PQEKRKNVA |
| 1033 | PQFTYRKYP |
| 1034 | QAAFHLKNA |
| 1035 | QATTKRTIF |
| 1036 | QCNVRTKMF |
| 1037 | QDFRLSSGM |
| 1038 | QEKCNRRGC |
| 1039 | QFAKQVKWF |
| 1040 | QGFMVMKAV |
| 1041 | QGKVKERTM |
| 1042 | QGRQVLGRE |
| 1043 | QILIKDCFS |
| 1044 | QKQKRTRAQ |
| 1045 | QMSGRCTCR |
| 1046 | QNRAIRSYT |
| 1047 | QPKFFYRSA |
| 1048 | QQLDATHER |
| 1049 | QQTKRSCMA |
| 1050 | QRKDKQTQH |
| 1051 | QSFRFWKHH |
| 1052 | QSKFKIPYC |
| 1053 | QSKLRLTEP |
| 1054 | QSQSTKAVW |
| 1055 | QSRQTGKFW |
| 1056 | QTCRKDRHF |
| 1057 | QTMKQIRAT |
| 1058 | QTRISRWDG |
| 1059 | QVFLKIRQE |
| 1060 | QVHNRLTQL |
| 1061 | QVHPSRWIR |
| 1062 | QVKMGKPNH |
| 1063 | QVMRQNTIT |
| 1064 | QYTRFNHVP |
| 1065 | RFKVWTCSC |
| 1066 | RFYDAMQMH |
| 1067 | RHDKQANGS |
| 1068 | RKNPTTCYS |
| 1069 | RKPHSGWRC |
| 1070 | RKRFTSMDP |
| 1071 | RLEDVHGNK |
| 1072 | RNKEWTGKG |
| 1073 | RQLPMNCCE |
| 1074 | RVNPELHMG |
| 1075 | SAKMQQISL |
| 1076 | SARDYREIA |
| 1077 | SARISAIGP |
| 1078 | SCKFKKSFF |
| 1079 | SCKHVVKVP |
| 1080 | SFIRTHRSW |
| 1081 | SFKMMTRQA |
| 1082 | SFKQKAREY |
| 1083 | SFRPTTWKQ |
| 1084 | SGSHYRRFP |
| 1085 | SHKFTGRCG |
| 1086 | SLNQFVMHE |
| 1087 | SQKHHTRSH |
| 1088 | SQKIVHYTN |
| 1089 | SSAQWLACD |
| 1090 | SSKFMACAS |
| 1091 | SSKLVSKTW |
| 1092 | SSRFLCHNE |
| 1093 | STKAWRYTG |
| 1094 | STKQAKRTA |
| 1095 | STTHLLRYP |
| 1096 | SVKLIGSEW |
| 1097 | SVVWPAQVE |
| 1098 | SWGYCMLDQ |
| 1099 | SWHMKMIAG |
| 1100 | SWKGMGKAD |
| 1101 | TAKITGSGS |
| 1102 | TAVRWMNGR |
| 1103 | TEYTRKKVP |
| 1104 | TGKSKSRMV |
| 1105 | TGKSKTWMP |
| 1106 | TGMRMPHGG |
| 1107 | TGTLTRWGP |
| 1108 | THFRKNWES |
| 1109 | TIANSRSHM |
| 1110 | TIKLSRFEP |
| 1111 | TIKVGHSRC |
| 1112 | TIWQRSGVD |
| 1113 | TKAIHNQSI |
| 1114 | TLKAYNHQI |
| 1115 | TMGFKTRYQ |
| 1116 | TMRGQPFGV |
| 1117 | TPDKRKWAQ |
| 1118 | TPILAGLVY |
| 1119 | TPKCIKWQQ |
| 1120 | TPRFHNKVP |
| 1121 | TPRSTRFCA |
| 1122 | TQHSLKAMM |
| 1123 | TSGWYKKSY |
| 1124 | TSKVKMWEY |
| 1125 | TSKVRSKCP |
| 1126 | TSVPHKWKR |
| 1127 | TTHLKLAAA |
| 1128 | TTKDGRAFE |
| 1129 | TTKEKKKDW |
| 1130 | TTKMGKWAA |
| 1131 | TTKPYQSIR |
| 1132 | TTKSRGFIP |
| 1133 | TTLRRHCCC |
| 1134 | TTLRTIKQA |
| 1135 | TTRTNSICS |
| 1136 | TVFYRHHHL |
| 1137 | TVGLGRRGA |
| 1138 | TVKSHTPMA |
| 1139 | TVKSREHSM |
| 1140 | TVLNYRRGF |
| 1141 | TVNVNKKAY |
| 1142 | TVRLQSYAQ |
| 1143 | TVRTLFKGN |
| 1144 | TVVAKTLRA |
| 1145 | TWRFNNHLC |
| 1146 | TYFKVMKDS |
| 1147 | VARPNVHQE |
| 1148 | VASSPRVCP |
| 1149 | VCVGRRAER |
| 1150 | VFKQFGKRE |
| 1151 | VGTSARKIG |
| 1152 | VLAPYRNCE |
| 1153 | VMVKRLATG |
| 1154 | VRQRPKFRP |
| 1155 | VSRTRIDSH |
| 1156 | VTKPKTVAD |
| 1157 | VTKWKNSGG |
| 1158 | VTMSRRSVW |
| 1159 | VTRSCCSFG |
| 1160 | VVKGMVCPC |
| 1161 | VVMQNNVAE |
| 1162 | WIEYISWWG |
| 1163 | YNFPKSAAE |
| 1164 | YNKSYRETG |
| 1165 | YPKPTRRDC |
| 1166 | YQKPSQTWS |
| 1167 | YSNLKSKAQ |
| 1168 | YTRGCHGCS |
| 1169 | YVSYARQSV |

In some embodiments, a retina tissue-tropic VP capsid polypeptide comprises a 581 to 589 region having at least 75% sequence identity to any one of SEQ ID NO: 14-SEQ ID NO: 1169. In some embodiments, a retina tissue-tropic VP capsid polypeptide comprises a 581 to 589 region having at least 77.7% sequence identity to any one of SEQ ID NO: 14-SEQ ID NO: 1169. In some embodiments, a retina tissue-tropic VP capsid polypeptide comprises a 581 to 589 region having at least 85% sequence identity to any one of SEQ ID NO: 14-SEQ ID NO: 1169. In some embodiments, a retina tissue-tropic VP capsid polypeptide comprises a 581 to 589 region having at least 88.8% sequence identity to any one of SEQ ID NO: 14-SEQ ID NO: 1169. In some embodiments, a retina tissue-tropic VP capsid polypeptide comprises a 581 to 589 region having a sequence of any one of SEQ ID NO: 14-SEQ ID NO: 1169. In some embodiments, a retina tissue-tropic VP capsid polypeptide comprises a 581 to 589 region having one or two amino acid substitutions relative to any one of SEQ ID NO: 14-SEQ ID NO: 1169. In some embodiments, a retina tissue-tropic VP capsid polypeptide comprises a 581 to 589 region having one or two conservative amino acid substitutions relative to any one of SEQ ID NO: 14-SEQ ID NO: 1169.

TABLE 4 provides additional 581 to 589 region sequences predicted to confer retina tissue tropism or preference, based on a primary screen of a recombinant AAV5 capsid library.

TABLE 4
Additional Exemplary 581 to 589 Region
Sequences for Retinal Targeting

| SEQ ID | |
|---|---|
| NO | 581 to 589 Region |
| 1170 | KSRKVKCLK |
| 1171 | KKKYKGHIP |
| 1172 | KKRKKILFM |
| 1173 | KTKCTKTKN |
| 1174 | KRKKAKVIE |
| 1175 | KQKIKKTAF |
| 1176 | KKCGKKRHY |
| 1177 | LCKNKKMKN |
| 1178 | KKKRKGQCP |
| 1179 | KQKTKHWMK |
| 1180 | CSRKAKFKK |
| 1181 | KAKNKACKK |
| 1182 | AKKKTLITK |
| 1183 | RKKKGKPSA |
| 1184 | HRRRAQYRS |
| 1185 | KGKKIKLMM |
| 1186 | TKKPCKYKS |
| 1187 | KKKTKTQIP |
| 1188 | KKTKGKSGT |
| 1189 | CRRRRKIHE |
| 1190 | IKTRKKWKY |
| 1191 | KAKFKKWAS |
| 1192 | KCRKKVKVA |
| 1193 | KKSVKKALV |
| 1194 | KSRKKQVKH |
| 1195 | YMKKHKKGQ |
| 1196 | KKTLKKWLP |
| 1197 | RRRRASCAE |
| 1198 | KGKKRKTAM |
| 1199 | KQKKKQNHQ |
| 1200 | KKYNKKGMW |
| 1201 | VVKHKKKVC |
| 1202 | KKKCKNIFA |
| 1203 | KLNKWKSKQ |
| 1204 | KKKQKCVRG |
| 1205 | AKKAKRWHK |
| 1206 | KKRKLFVKN |
| 1207 | KRKKKNHCR |
| 1208 | KIKKKFDTI |
| 1209 | KVKIKQIKQ |
| 1210 | KKKIKMFCT |
| 1211 | IYKWKKKGT |
| 1212 | KRKGKKFCS |
| 1213 | KKKTCKFEG |
| 1214 | KTRMKRIQV |
| 1215 | QAKKKKQWD |
| 1216 | MKKKQKNVA |
| 1217 | KKRKAHLQW |
| 1218 | IKTKKKWGY |
| 1219 | KCKAFKGYT |
| 1220 | KVHKKKGWH |
| 1221 | RFRKFRRCT |
| 1222 | KSKKTKCLD |
| 1223 | KKKQKQCYF |
| 1224 | KQKKKPAIW |
| 1225 | AKKRRKGSM |
| 1226 | KVGKHLKKG |
| 1227 | TRRRRWCGN |
| 1228 | KKKKKRIFR |
| 1229 | KPKMGKYTK |
| 1230 | ARKKKAGKC |
| 1231 | MTKMKKKMC |
| 1232 | KRARLKHSQ |
| 1233 | KGKKRAKTG |
| 1234 | KQKFFKHAM |
| 1235 | KKKRNRDGL |
| 1236 | RSKQFRVHD |
| 1237 | KRRKPCWRQ |
| 1238 | QTKQKKWKN |
| 1239 | KQRKHKVDT |
| 1240 | KAKSKKGLM |
| 1241 | EVKKKKFMP |
| 1242 | KVKSHKLGQ |
| 1243 | KKKRVQSRD |
| 1244 | KAKRCKASC |
| 1245 | VQKKGWQKK |
| 1246 | KRKWKRDCF |
| 1247 | KAKKSKHDR |
| 1248 | KCKMMKIKC |
| 1249 | KKKKFPECG |
| 1250 | RYKHHRFLP |
| 1251 | KKHRKGQMT |
| 1252 | EWKKKKCFF |
| 1253 | VKKRSKVQA |
| 1254 | RRRRILHCP |
| 1255 | MKKRQRGAS |
| 1256 | KKHKKQLEN |
| 1257 | QTKPKKTKC |
| 1258 | KKKMQCIKC |
| 1259 | QQKEKKWKE |
| 1260 | KTKLYKYCY |
| 1261 | PKKFKVHKC |
| 1262 | YARRAKSTE |
| 1263 | KPVKKQFKE |
| 1264 | RCRSRRYPE |
| 1265 | KRRYRKHNY |
| 1266 | HKRTLKHWF |
| 1267 | RTRRLSPRE |
| 1268 | SKRKKGCVG |
| 1269 | TKPKKKWWA |
| 1270 | QSRKAKRIQ |
| 1271 | RFKSRRSRA |
| 1272 | LVGKKKTGK |
| 1273 | MVKLKKKHH |
| 1274 | KAKYWKHRF |
| 1275 | RPKKVKNCE |
| 1276 | HCRKRRHFH |
| 1277 | VKKMKIWKH |
| 1278 | KVRSKKMWS |
| 1279 | ITKSKKKWT |
| 1280 | KARKKMWSC |
| 1281 | KVKPKSNKS |
| 1282 | KIKRIIVAH |
| 1283 | KKAHAKKVM |
| 1284 | KKKQKRVGH |
| 1285 | RKKGGKLVC |
| 1286 | TKRKHKPVN |
| 1287 | RRKSCRFHR |
| 1288 | KKKYHPKWT |
| 1289 | AAKRQKIQW |
| 1290 | FVRKLKTTT |
| 1291 | YCKLYKKKG |
| 1292 | RRVRKHQRE |
| 1293 | PKLKKKGFS |
| 1294 | CPKNKKKVP |
| 1295 | FKKCKGTKS |
| 1296 | RQRKFKCMQ |
| 1297 | KKHRFKLPV |
| 1298 | DAKKDRYKK |
| 1299 | KKHRSHPSQ |
| 1300 | KMRKTKLWG |
| 1301 | EKKKYCHKT |
| 1302 | DVKKKKWPP |
| 1303 | QVKSKKKQI |
| 1304 | KIRKTKVYN |
| 1305 | KIIKIKKFW |
| 1306 | MKRKLKYAN |
| 1307 | KAKSRQFCQ |
| 1308 | ARARRRYCN |
| 1309 | KKRGYHFQH |
| 1310 | NKKQKCQKV |
| 1311 | VTCKKKSKS |
| 1312 | CIKKCKQWY |
| 1313 | KAQKHLKKC |
| 1314 | VRRKVRRTP |
| 1315 | KSRQKKGPH |
| 1316 | KRKTAKYFQ |
| 1317 | KYKSRKVNS |
| 1318 | YFRARRRYS |
| 1319 | TRRKKKSWN |
| 1320 | KKKPKSHLG |
| 1321 | KRKKSGHFY |
| 1322 | NRRVSRWRK |
| 1323 | QRQKIRRRT |
| 1324 | KMPKQKFKN |
| 1325 | KKNKKCKKP |
| 1326 | DTKRKKSCA |
| 1327 | SKKRHRSHV |
| 1328 | KYKQGGKQY |
| 1329 | KSARKKWSC |
| 1330 | HKKHFKKPL |
| 1331 | GRRKYRADR |
| 1332 | SVKHYKKHQ |
| 1333 | NTKRLKCKQ |
| 1334 | EKKFKKCGG |
| 1335 | KKRYYHRPC |
| 1336 | KGRKIRTQT |
| 1337 | VKKRPKWSK |
| 1338 | QIRRKKAYY |
| 1339 | TCKRKKLNA |
| 1340 | KARSLSRWL |
| 1341 | KLKYIKRSA |
| 1342 | KQRYRKHGQ |
| 1343 | RWRRRQKLQ |
| 1344 | DIKFKKKIF |
| 1345 | HKKKCQNCN |
| 1346 | RMKRYHYTG |
| 1347 | KVKCIKQLG |
| 1348 | RAKRYKHTW |
| 1349 | KGKCMKNAQ |
| 1350 | KNKKRVRGV |
| 1351 | KLKKQPFKG |
| 1352 | LKRKHRRTG |
| 1353 | MKTKTHCKS |
| 1354 | FYKRMRMHN |
| 1355 | HRKFKKQAY |
| 1356 | CKSRKRRGQ |
| 1357 | HKKRTVAWM |
| 1358 | KAKFCRKPQ |
| 1359 | KHRKFRCMT |
| 1360 | RRGRRWVPQ |
| 1361 | CFRKYKNKG |
| 1362 | KWKSKGKNC |
| 1363 | HKHRKQRYV |
| 1364 | RTRRYRHWL |
| 1365 | KTRKGRSNH |
| 1366 | PCRRRRLWG |
| 1367 | KMRKAHVTG |
| 1368 | SRKKCKVCY |
| 1369 | KSKSKYWGV |
| 1370 | KKKSKLFWN |
| 1371 | KKMKCKEAR |
| 1372 | VGKLKKKWQ |
| 1373 | SVKSKKSGL |
| 1374 | FSRKKKLYY |
| 1375 | VNYKKKGKC |
| 1376 | KWRKMKGCY |
| 1377 | KMKSKADKG |
| 1378 | ARKLKRTRF |
| 1379 | KYRKTNVFW |
| 1380 | KNKKEHKSD |
| 1381 | KGKHRKFYA |
| 1382 | KRKLGKFNG |
| 1383 | KKARKGQPW |
| 1384 | TAKRQRIKH |
| 1385 | KRKRTNYWW |
| 1386 | MQRKKKCTG |
| 1387 | KKEKKIWTC |
| 1388 | KQRKRFNSW |
| 1389 | KKKALGFRS |
| 1390 | KVRPKRAGP |
| 1391 | EHRRRRIVG |
| 1392 | KKKKCQAAP |
| 1393 | VKKIKHVKG |
| 1394 | RVRPTRKQC |
| 1395 | KERKQKEMM |
| 1396 | KRKKFMNAR |
| 1397 | RIRKMTRTQ |
| 1398 | KKKPNRIAA |
| 1399 | KKKTIRHNP |
| 1400 | VKKPRGRHP |
| 1401 | RCRKFKSDM |
| 1402 | KSKYAKNQA |
| 1403 | KTCKTKTMF |
| 1404 | HKKCKSQKS |
| 1405 | KGRKYWCGG |
| 1406 | KHKRRTFYP |
| 1407 | KKSKEKAPF |
| 1408 | KTCKKSKIV |
| 1409 | RKKRWHVQY |
| 1410 | KRSRLKRSH |
| 1411 | SKKRYMYKS |
| 1412 | SKVRKRFRV |
| 1413 | CKAKRKSGY |
| 1414 | VCRRIKGRW |
| 1415 | HKTHKKKMT |
| 1416 | KQKKYVMTA |
| 1417 | KRRKQYHCK |
| 1418 | KKATFKQAK |
| 1419 | RIKCKKTTL |
| 1420 | FKKRYLLWT |
| 1421 | KKDRHKHKS |
| 1422 | LARKFKLWH |
| 1423 | TCCKKKKWM |
| 1424 | RRRKIMGHS |
| 1425 | KIKPCKKNH |
| 1426 | KCKQYKMSV |
| 1427 | KQKLOKQAP |
| 1428 | KIKQCKRAW |
| 1429 | KRKKQSVYN |
| 1430 | HCKYHKKGF |
| 1431 | GRRCCRRGF |
| 1432 | TKRLKTRRD |
| 1433 | QRPRKRKGG |
| 1434 | KAKCSKSYC |
| 1435 | RRRKGLMIQ |
| 1436 | GKKKSKLGE |
| 1437 | RKKALKRHG |
| 1438 | SSKRRKQCF |
| 1439 | KMKFQKYAC |
| 1440 | KIRKWSAFE |
| 1441 | KKRRTVHPN |
| 1442 | KAGWLKKEK |
| 1443 | KHKTKHNWE |
| 1444 | DHKRQKRQP |
| 1445 | KYKHRGGYT |
| 1446 | EKKVKKEQW |
| 1447 | MSKRFKTMG |
| 1448 | PMKKKEYKC |
| 1449 | QKKWKFKTC |
| 1450 | EKKPQKKQY |
| 1451 | RGKARRSYA |
| 1452 | KIKRNRSNM |
| 1453 | SKKCGKKDF |
| 1454 | YWKKIKRWN |
| 1455 | KYKQVKKLA |
| 1456 | KQRKCYYKT |
| 1457 | HRRKTRYFR |
| 1458 | NCKGKCVKK |
| 1459 | KTKFRGSKS |
| 1460 | FKRKTRIDG |
| 1461 | KTKCFKGGF |
| 1462 | RVKHYRHYQ |
| 1463 | DKKPKKQYW |
| 1464 | HRKSKKWQP |
| 1465 | RKGKKDCKG |
| 1466 | KSRKHIRTW |
| 1467 | AKQRQRHQP |
| 1468 | MTKSKKRWN |
| 1469 | RRRRYMLIA |
| 1470 | CKKTKTHKF |
| 1471 | KQVKKKKES |
| 1472 | HTKRKGAWM |
| 1473 | KRMKRSRND |
| 1474 | KQRRKCVCY |
| 1475 | KKKCQSFGS |
| 1476 | KKKASGWTT |
| 1477 | EVKSKKRQP |
| 1478 | CRRRLKCGE |
| 1479 | KAKSRGGYN |
| 1480 | LAKYKKSYH |
| 1481 | KCKKCYKCD |
| 1482 | LKKKTKPCG |
| 1483 | EARRKKGWH |
| 1484 | CPRKSKFCM |
| 1485 | MNARVKRVG |
| 1486 | KCKFVKAHT |
| 1487 | KKRNKAQYV |
| 1488 | MVKRNKHYG |
| 1489 | KARWKGRVQ |
| 1490 | CSKKRKAGT |
| 1491 | GVRKTKWCN |
| 1492 | KQKFRGMRY |
| 1493 | VKRKHKFEN |
| 1494 | KGRKWGGTG |
| 1495 | IVKRLKQGA |
| 1496 | SKRKLGQVN |
| 1497 | KYKSQKWTV |
| 1498 | KRTRKKRMV |
| 1499 | MYKLKKTAV |
| 1500 | HRRKWLYPA |
| 1501 | KKKTYSCLS |
| 1502 | KRKRLPYGM |
| 1503 | KRRKNVWCP |
| 1504 | HKRKRYVAV |
| 1505 | KVRVHKMVC |
| 1506 | KWRKEKMHY |
| 1507 | KQKQKKVGP |
| 1508 | KRKAKQGCN |
| 1509 | TNKSKKSQN |
| 1510 | VKRRKIYGA |
| 1511 | KIRLRKGVS |
| 1512 | KCKIVKQMG |
| 1513 | KCRKQHHTM |
| 1514 | RAKIHRYYT |
| 1515 | GKRKHMPCP |
| 1516 | KCKKDRKQC |
| 1517 | QKHRTKKWS |
| 1518 | IKKKFGRCC |
| 1519 | LKRKDKFGE |
| 1520 | KSRKAWLCM |
| 1521 | RFRGHRCWR |
| 1522 | EKRKSKCCP |
| 1523 | KRRKQMHRV |
| 1524 | KQKWWKYGC |
| 1525 | KQKYGKFYN |
| 1526 | NGKLFKKHV |
| 1527 | TTKSFKWRQ |
| 1528 | KKRIHRHEY |
| 1529 | HTKSKKFFS |
| 1530 | KSRSKQRQC |
| 1531 | NVRRYKTHI |
| 1532 | VKKRYHRQQ |
| 1533 | HIRKAKKQC |
| 1534 | KRRRIRHLM |
| 1535 | KKKIGTKCF |
| 1536 | ESKRKRSMN |
| 1537 | YCKLKKIHQ |
| 1538 | NKMSKKKVC |
| 1539 | FFKKRRVCC |
| 1540 | RKKVCRFSN |
| 1541 | SVKMMKTLP |
| 1542 | KVRWKHGGA |
| 1543 | ITRKNKLWQ |
| 1544 | VIRRMRNFF |
| 1545 | RRRRLTPSA |
| 1546 | KNRRKKYAC |
| 1547 | KIRQKCMKF |
| 1548 | ACKNKKKDG |
| 1549 | CFKRRKYMV |
| 1550 | KAKKRANDY |
| 1551 | RRKHMRSKS |
| 1552 | GSRRRKHTH |
| 1553 | KVKMRGFWD |
| 1554 | KGRKRHQYM |
| 1555 | RYKSCKAKG |
| 1556 | RRQRSRYWF |
| 1557 | HIRKQKNNN |
| 1558 | GSRKRKMCT |
| 1559 | RYKCYREYP |
| 1560 | CKKFAKKHD |
| 1561 | KDKMCKAKG |
| 1562 | KRRKTHTKV |
| 1563 | SGKKGKSDN |
| 1564 | HKRKPYSWM |
| 1565 | RHRKKVTHD |
| 1566 | IVKSKKSYI |
| 1567 | QSKTTKKVC |
| 1568 | TKTAKKCKG |
| 1569 | RKGKCKFVY |
| 1570 | KTKRSWCSG |
| 1571 | SSKKPKHCA |
| 1572 | RLRRMWYSG |
| 1573 | HKRRNNWWP |
| 1574 | KCKLQKNNV |
| 1575 | KAKSRRYVE |
| 1576 | KRKFYKCNM |
| 1577 | KKKYCIGHM |
| 1578 | KKKANRATY |
| 1579 | DFKKCKMWQ |
| 1580 | KRKALKVDS |
| 1581 | KKQRFRAAY |
| 1582 | YHRKGKHRN |
| 1583 | HVRSKRHMM |
| 1584 | KFGKQKTLC |
| 1585 | TKRRYHHHC |
| 1586 | KKHRGSFHN |
| 1587 | RMKTRKTCS |
| 1588 | KMKCSKHKD |
| 1589 | GNRRRKYWG |
| 1590 | CYRRYKSYC |
| 1591 | EWKRYRVQS |
| 1592 | LKKTHKKSH |
| 1593 | KLKNKHWFC |
| 1594 | ICHRRRRCH |
| 1595 | PKRKQKWAS |
| 1596 | KHKRSYQAH |
| 1597 | KLKSNKQCC |
| 1598 | KRKKNTFVV |
| 1599 | KRRSYKWIN |
| 1600 | TARKTKQMI |
| 1601 | VARRKRHYT |
| 1602 | HKHRYRYLW |
| 1603 | STRRMTRGM |
| 1604 | MVRRYRSAA |
| 1605 | KAKVRQFSK |
| 1606 | MCKRVRTPP |
| 1607 | HRRKKVTCA |
| 1608 | HQKKQKWPH |
| 1609 | TRKRYVVYA |
| 1610 | KFRKANWPE |
| 1611 | KSRQYKRTW |
| 1612 | EMKKYKKWP |
| 1613 | KLKKKLSEE |
| 1614 | TFKKTKFIP |
| 1615 | CKKKTLAQV |
| 1616 | QKAKYKWCF |
| 1617 | GKRRMRSGW |
| 1618 | KKHKAGGQL |
| 1619 | MAKNKKCGL |
| 1620 | FVKSRRFPN |
| 1621 | KCRKIAHNC |
| 1622 | HRKPRRVHS |
| 1623 | TRRRTKISQ |
| 1624 | RVRHKRRHW |
| 1625 | KVRNWKSMA |
| 1626 | FRRTRRYWS |
| 1627 | VTKYKKKAF |
| 1628 | YKRKQQCAQ |
| 1629 | KRKYHKLCN |
| 1630 | KKRHRVSHT |
| 1631 | LMKAKKKDT |
| 1632 | KYKNKKIVG |
| 1633 | IVKKHKKAE |
| 1634 | DVKFCRYYR |
| 1635 | GYPRRGRRT |
| 1636 | VKKGPRTEK |
| 1637 | TNKSKKCMW |
| 1638 | AFKRFRSDC |
| 1639 | KAKQKGFMN |
| 1640 | AIKAKKVGF |
| 1641 | KTKRRMLRQ |
| 1642 | KGKKHQFSP |
| 1643 | KKKSHKLSM |
| 1644 | KSKIQKIHM |
| 1645 | MSRKKMWRS |
| 1646 | KKYRFNWNH |
| 1647 | KKKCQQCCH |
| 1648 | KIKAFRWQG |
| 1649 | QLKKFKYKA |
| 1650 | RTRKANHGT |
| 1651 | EARKAKMYA |
| 1652 | SVKKWKQRE |
| 1653 | AKGKPKVMC |
| 1654 | RGRKRWVNH |
| 1655 | KSKCFKLQS |
| 1656 | KRQSRKCRG |
| 1657 | KGRGTKYKY |
| 1658 | KIKRMKARC |
| 1659 | MVKSKKKNF |
| 1660 | AVKYYRRQQ |
| 1661 | KHKWKRTSF |
| 1662 | KKKSCICVY |
| 1663 | KGRKCGPMM |
| 1664 | MIRKAKINC |
| 1665 | KAKTKQQWS |
| 1666 | KKSKCHQRC |
| 1667 | TAKRQGLKQ |
| 1668 | KYRQKRCVQ |
| 1669 | KQKVQCSHK |
| 1670 | KWKRHFKGS |
| 1671 | HKKYWKNSA |
| 1672 | KVRSKQWVW |
| 1673 | QKRKYVHKG |
| 1674 | RKKFIKMQC |
| 1675 | PRRRTRLTH |
| 1676 | KIRPKRLEM |
| 1677 | KQKVTKIGS |
| 1678 | FKRKTAVKP |
| 1679 | KVKSGKRAQ |
| 1680 | KLRAARYRG |
| 1681 | KVKPRGMHR |
| 1682 | KVRRHKGVL |
| 1683 | KYKAMKTCG |
| 1684 | LMKFKKQFC |
| 1685 | KKKQKTAHH |
| 1686 | KAKSKWHNF |
| 1687 | KIKRYGSCD |
| 1688 | DAKKWKKCH |
| 1689 | KRRRSKYET |
| 1690 | RIRFIKYAH |
| 1691 | RVKWTRQNQ |
| 1692 | MGRKLKSKE |
| 1693 | KTRKVHIWT |
| 1694 | TKKITKHSS |
| 1695 | VGKRSKISW |
| 1696 | MPKRKRCAS |
| 1697 | NHKSYKRQH |
| 1698 | LKNKTKKQA |
| 1699 | NYKKFKCFQ |
| 1700 | RHRHDRFRH |
| 1701 | KLKIVKWAH |
| 1702 | SYKVKKSAY |
| 1703 | AGKKFKCSI |
| 1704 | SARKKGQVI |
| 1705 | RRRLQKSNS |
| 1706 | MAKTKKYWQ |
| 1707 | KRRKYWRFE |
| 1708 | LWKYKKKFD |
| 1709 | KWKCGKFYS |
| 1710 | KKRSVKSKG |
| 1711 | IAKKHKSGM |
| 1712 | RVNKMKTYP |
| 1713 | RRKSTKNHK |
| 1714 | LARKCKWVC |
| 1715 | SVKKAKWYN |
| 1716 | KWRAIKGQS |
| 1717 | KKTVKRRTG |
| 1718 | KIKMLKGVM |
| 1719 | VHKLNKMKW |
| 1720 | KGKVYKDKQ |
| 1721 | KYKLRKTLF |
| 1722 | IVKLKKCIM |
| 1723 | RKRASKVCN |
| 1724 | KKTRRKKEF |
| 1725 | KKRKLDFCS |
| 1726 | KAARKKSMC |
| 1727 | KARKCTPSH |
| 1728 | MNKKTKYHH |
| 1729 | RKKSRYCHP |
| 1730 | QAKQKKHYH |
| 1731 | TVKKHKDSN |
| 1732 | NVKSKKKFW |
| 1733 | SVKRCKAYT |
| 1734 | KHRKSIWVQ |
| 1735 | NKRKKSQDV |
| 1736 | RRRGWKERC |
| 1737 | KRKYRQNGN |
| 1738 | KFKTPKGWT |
| 1739 | KKKFGGAHW |
| 1740 | AVKRRKCPG |
| 1741 | KRIRKKISS |
| 1742 | IKKSPKKRM |
| 1743 | KARNSRYSI |
| 1744 | MVKPRRSIA |
| 1745 | YTKSKKSAL |
| 1746 | HRKRWHAMH |
| 1747 | KRKNAGRGV |
| 1748 | KAHKRKQAN |
| 1749 | KKARSHWRC |
| 1750 | KMRPSKFHS |
| 1751 | RVRKARSFP |
| 1752 | KARKYGRYL |
| 1753 | RKKRQACWF |
| 1754 | SQKAYRGHS |
| 1755 | HIRKARYDH |
| 1756 | HAKQKHQKQ |
| 1757 | RTKSCKWKH |
| 1758 | KSRKQYAQW |
| 1759 | KGGKKRCKV |
| 1760 | DKPKKKYAA |
| 1761 | KLKSCKQCS |
| 1762 | HTKAYRRES |
| 1763 | KKRLKPTSG |
| 1764 | HKKYRKQWA |
| 1765 | KTKFRKNCT |
| 1766 | TLKCKRKGM |
| 1767 | KSRKHANSG |
| 1768 | KRKRCMIDH |
| 1769 | HKRRITYWM |
| 1770 | ISKAKKTNM |
| 1771 | KFRNRRCAY |
| 1772 | TTKYHKKCW |
| 1773 | RTRKWKWWP |
| 1774 | KTKQLKCTK |
| 1775 | NFRKYKMHN |
| 1776 | KRRRSPYMS |
| 1777 | NAKWIKKWY |
| 1778 | KYRIRRHMC |
| 1779 | GSKNKKQMC |
| 1780 | MLKKGKHHP |
| 1781 | YWKRTRFQF |
| 1782 | IMRKFKRES |
| 1783 | KRKRVAAKD |
| 1784 | KARKQWMHS |
| 1785 | RSKIYRTWE |
| 1786 | SCKKKRMIF |
| 1787 | KTRLKKITR |
| 1788 | TARKRKHTT |
| 1789 | HFKCQKWRF |
| 1790 | KGQRKKSGL |
| 1791 | KSKGKQWYT |
| 1792 | KRRPIRDCF |
| 1793 | TVKKSRLRN |
| 1794 | SMRKFKWKE |
| 1795 | VGKGKKQMC |
| 1796 | QKGLKKHKP |
| 1797 | PSRKLKRLC |
| 1798 | SVRRNKHVY |
| 1799 | FMKRCKMQT |
| 1800 | NKKKFHHAQ |
| 1801 | TNKKFKSVS |
| 1802 | TRRKRPCYP |
| 1803 | NRKRQRNWE |
| 1804 | RAKTSRTEV |
| 1805 | YKPRKRNAT |
| 1806 | CKKKLSRHQ |
| 1807 | KVRKSRGQS |
| 1808 | HQKGKKRGL |
| 1809 | SKKPMKKCC |
| 1810 | KQKKSHSHG |
| 1811 | RIKLQRAMH |
| 1812 | KQKKQLDTT |
| 1813 | THKCKKTGV |
| 1814 | RLKMHRAMC |
| 1815 | KHKCSKFYQ |
| 1816 | NVRNKRAHP |
| 1817 | NKHKPKYHL |
| 1818 | SAKLKKRCN |
| 1819 | KGKNKIGDN |
| 1820 | KLKSKTFAS |
| 1821 | QCKRTKHYK |
| 1822 | YAKPKKYCL |
| 1823 | KRSRMRCFW |
| 1824 | QKKQLKAAA |
| 1825 | LKLRKKHGQ |
| 1826 | KTKKAYVVS |
| 1827 | RSMRIRRDY |
| 1828 | KKKTNTHLG |
| 1829 | KRKYNRSWC |
| 1830 | RRRKQDHVC |
| 1831 | CVKSKKTGY |
| 1832 | SCKSKKSQH |
| 1833 | LRKQKKWTG |
| 1834 | KRRKCPQYE |
| 1835 | KIKNRKSWE |
| 1836 | AGRKAKRVW |
| 1837 | KRRHGHIHS |
| 1838 | KMKNMTMWM |
| 1839 | KRRHNKNYD |
| 1840 | IRMRKKVGG |
| 1841 | VRKHIKRQY |
| 1842 | KKKTSWMGM |
| 1843 | HKRFYKVMW |
| 1844 | HKKRVCRMG |
| 1845 | KVCKFKQSA |
| 1846 | WFKIKRRRS |
| 1847 | KRRNKHPYW |
| 1848 | KTKCNRKAT |
| 1849 | DCKKKGALS |
| 1850 | KGSFKKKMP |
| 1851 | KGRYKQRSS |
| 1852 | HKQRKQQWV |
| 1853 | HKKRENYVF |
| 1854 | VKRKPPRGM |
| 1855 | KAKLYRQFP |
| 1856 | KCRRARSMD |
| 1857 | KATKKFAAT |
| 1858 | SKCNKKKWF |
| 1859 | KSSRKRIYQ |
| 1861 | RRRMRKIAF |
| 1862 | KNKKASIIG |
| 1863 | RKRMAKQPG |
| 1864 | NVRKHKCIH |
| 1865 | KWKRTKIVW |
| 1866 | VGRFGRVGT |
| 1867 | CVKTKKTAP |
| 1868 | KKRSFNCIF |
| 1869 | MQKKTKGCY |
| 1870 | SVKYKKQTN |
| 1871 | KKHKHQINC |
| 1872 | KCKPVRRAE |
| 1873 | IYRRYMRAH |
| 1874 | QCRRGRHQQ |
| 1875 | HKRKWFDKP |
| 1876 | KQRKLGLGE |
| 1877 | KNKIYREIQ |
| 1878 | KQKQTKFGN |
| 1879 | RVKIPIQIR |
| 1880 | NKHRYKCKY |
| 1881 | RKRYSKLNS |
| 1882 | NKHKMRHHN |
| 1883 | KRQRKIWRQ |
| 1884 | KVRRCKLKY |
| 1885 | CSKKSKRQS |
| 1886 | HRKTVKWNA |
| 1887 | KGKSPKLTG |
| 1888 | HIRKHWGSR |
| 1889 | KSKSKSVHD |
| 1890 | NTRKLKNCW |
| 1891 | RNKRRAWNN |
| 1892 | FCKSKRINC |
| 1893 | KRKGQGRTC |
| 1894 | NKCGKKKQP |
| 1895 | RCRRQKVPY |
| 1896 | EKRKKGIHA |
| 1897 | LTRKYKYCT |
| 1898 | KTRRPHQTV |
| 1899 | QVKHFKWTS |
| 1900 | KVKFSKWYK |
| 1901 | QKCKVKYAY |
| 1902 | VKRLKKAVC |
| 1903 | SEKRMKRKG |
| 1904 | KCGRKPINK |
| 1905 | LQKAKKHWQ |
| 1906 | DFKVRKKYP |
| 1907 | KVRHWKCSM |
| 1908 | KSRKQANAE |
| 1909 | KNRLKKRSP |
| 1910 | KHRMFQRSW |
| 1911 | HMRKAKQFT |
| 1912 | RERKLKFQD |
| 1913 | KYKSGKHHW |
| 1914 | KRRHIRRPG |
| 1915 | KWKFKPFSQ |
| 1916 | KRKKQQGYH |
| 1917 | LKRRSPHQY |
| 1918 | IQRKSKCGC |
| 1919 | KGKMKRLET |
| 1920 | VIKFKKCYS |
| 1921 | SVKPRKKAS |
| 1922 | KGGKNKVMV |
| 1923 | MAKSCKCKQ |
| 1924 | KKDKKHLTF |
| 1925 | KSKPRGRGT |
| 1926 | CSKLMKKKP |
| 1927 | KSRKQGISC |
| 1928 | KTKEKKLCY |
| 1929 | QGKSKKWNF |
| 1930 | FKKRMHRKC |
| 1931 | KTKKHGNQQ |
| 1932 | KKKLYWSQP |
| 1933 | TVKKAKRFS |
| 1934 | HKHRSIVKC |
| 1935 | HNKKMRLSG |
| 1936 | CYKSKKLCN |
| 1937 | KVKWRVKPE |
| 1938 | KARFIRHSA |
| 1939 | KKRVGPNVA |
| 1940 | KKNRKAKDY |
| 1941 | KKKWKEWLH |
| 1942 | CHRKVKIVC |
| 1943 | KKKFWTIGN |
| 1944 | MAKRPRLGT |
| 1945 | KLRTMKGLG |
| 1946 | QYKAYKKVQ |
| 1947 | TFRKCKGYW |
| 1948 | NKRKKAFDN |
| 1949 | KKKWTQHMS |
| 1950 | RQKAIKSFH |
| 1951 | KYRGWKSPG |
| 1952 | ANKKRRRAY |
| 1953 | VVKAKKNIM |
| 1954 | KKIKYRKTF |
| 1955 | KKKWQGQAP |
| 1956 | GAKWYKKGM |
| 1957 | RSRPSKQVV |
| 1958 | GTRRNRVQQ |
| 1959 | IRKKSRSKS |
| 1960 | KKKHLHGHS |
| 1961 | GLKKHKMAS |
| 1962 | SKRRTNRAV |
| 1963 | RQKKKMANE |
| 1964 | WRKRCKNTS |
| 1965 | LGRKQKLWH |
| 1966 | AVRYKRRQE |
| 1967 | KQRKSVGRQ |
| 1968 | KWKKQTCFL |
| 1969 | KGKRRWCKC |
| 1970 | CVRRTRWRN |
| 1971 | KRRKTPLHT |
| 1972 | MKKHHRQHW |
| 1973 | YTKSSRKGP |
| 1974 | KQRFFKTGS |
| 1975 | KLKHRGRWH |
| 1976 | HVKGHRRLV |
| 1977 | LKRKQRCQH |
| 1978 | DSKYRRSAR |
| 1979 | KQRKATICS |
| 1980 | EYKKKHKCG |
| 1981 | KIRPKKQVY |
| 1982 | CRKVHRNYC |
| 1983 | KARMKRQKR |
| 1984 | KLKKPRNCY |
| 1985 | KRKWWLMHS |
| 1986 | KIKMHGWRH |
| 1987 | KNKKAKYCS |
| 1988 | KIKKKQLDD |
| 1989 | KRARQKWQA |
| 1990 | RTKNRYWRV |
| 1991 | YKPRHLHKE |
| 1992 | KRPGKRKRN |
| 1993 | KIKLFKQGY |
| 1994 | IKKRMNSSL |
| 1995 | RRRHYKVNS |
| 1996 | KSKMSKHGA |
| 1997 | KLKEHKRMF |
| 1998 | AKKMLKRRG |
| 1999 | QAKIIKKGM |
| 2000 | NHRRKKCQF |
| 2001 | CRRKSKGQC |
| 2002 | KGRGMKWMH |
| 2003 | RWRLKKFQG |
| 2004 | RTRKWQKIW |
| 2005 | WKKRYSLQM |
| 2006 | CKKRYSHGQ |
| 2007 | DAKSKKRAA |
| 2008 | SKKRNQLIQ |
| 2009 | SPRPRKQAM |
| 2010 | VKKKAGLTQ |
| 2011 | HHRKPKYDT |
| 2012 | NRKARRCTD |
| 2013 | IAKWKKCTM |

In some embodiments, a retina tissue-tropic VP capsid polypeptide comprises a 581 to 589 region having at least 75% sequence identity to any one of SEQ ID NO: 1170-SEQ ID NO: 2013. In some embodiments, a retina tissue-tropic VP capsid polypeptide comprises a 581 to 589 region having at least 77.7% sequence identity to any one of SEQ ID NO: 1170-SEQ ID NO: 2013. In some embodiments, a retina tissue-tropic VP capsid polypeptide comprises a 581 to 589 region having at least 85% sequence identity to any one of SEQ ID NO: 1170-SEQ ID NO: 2013. In some embodiments, a retina tissue-tropic VP capsid polypeptide comprises a 581 to 589 region having at least 88.8% sequence identity to any one of SEQ ID NO: 1170-SEQ ID NO: 2013. In some embodiments, a retina tissue-tropic VP capsid polypeptide comprises a 581 to 589 region having a sequence of any one of SEQ ID NO: 1170-SEQ ID NO: 2013. In some embodiments, a retina tissue-tropic VP capsid polypeptide comprises a 581 to 589 region having one or two amino acid substitutions relative to any one of SEQ ID NO: 1170-SEQ ID NO: 2013. In some embodiments, a retina tissue-tropic VP capsid polypeptide comprises a 581 to 589 region having one or two conservative amino acid substitutions relative to any one of SEQ ID NO: 1170-SEQ ID NO: 2013.

Retinal Pigment Epithelium Tissue-Tropic AAV VP Capsid Polypeptides. Described herein are engineered AAV VP capsid polypeptides comprising a variant 581 to 589 region predicted to confer increased retinal pigment epithelium (RPE) tissue tropism, and predicted to confer increased retina tissue tropism compared to a wild type AAV VP capsid polypeptide (e.g., an AAV5 VP capsid polypeptide), based on a primary screen of a recombinant AAV5 capsid library. In some embodiments, an RPE tissue-tropic engineered AAV VP capsid polypeptide may also have tropism for choroid tissue. TABLE 5 provides 581 to 589 region sequences predicted to confer RPE tissue tropism or preference.

TABLE 5
Exemplary 581 to 589 Region
Sequences for RPE Targeting

| SEQ ID | |
|---|---|
| NO | 581 to 589 Region |
| 2014 | DCTFHDAEI |
| 2015 | TDEHVKMGP |
| 2016 | GTVWKNWPD |
| 2017 | NIEPTANCQ |
| 2018 | DEMAHYASS |
| 2019 | WINYTVKDR |
| 2020 | EFDQCTHGI |
| 2021 | QIYVTHQGP |
| 2022 | GTVNEYSWN |
| 2023 | VRYACLYEN |
| 2024 | WFDHNYCFG |
| 2025 | EYCPCGEVQ |
| 2026 | DRPCAVHEA |
| 2027 | VCVNRLECP |
| 2028 | TEQKQYESI |
| 2029 | TDNCKGECQ |
| 2030 | CNTQFMKRE |
| 2031 | MMIQNHCLS |
| 2032 | AVTAPTFRG |
| 2033 | FYTNAGCYC |
| 2034 | AQQSWEQIQ |
| 2035 | MHMNQCVID |
| 2036 | IHTIEQKCS |
| 2037 | IKLDVEMAG |
| 2038 | AHCTKTMQV |
| 2039 | MPQQNVAVP |
| 2040 | MQYFWHYAH |
| 2041 | AYESKIHTG |
| 2042 | MSLWCSEER |
| 2043 | LATAHSMRF |
| 2044 | VWYFYNEAP |
| 2045 | VQMAVLAYW |
| 2046 | VVLTQLEDS |
| 2047 | NQMFNNEYP |
| 2048 | HTYGSLAQY |
| 2049 | NTFMECVGG |
| 2050 | VKQMGCDIA |
| 2051 | HGQCALHTV |
| 2052 | MIGTFAYHQ |
| 2053 | VNPTKAEYA |
| 2054 | LMLGAYKEC |
| 2055 | MGEPTCEYR |
| 2056 | LTVMWGCCY |
| 2057 | PNHCYSTWN |
| 2058 | DLHAYICFI |
| 2059 | WVTMHNQLC |
| 2060 | GMEYYAHNW |
| 2061 | PPNVTVDQE |
| 2062 | QPDTVWHYA |
| 2063 | VSWVERACP |
| 2064 | CPPDHCYTA |
| 2065 | NTQSQACYI |
| 2066 | EYWFTHAEN |
| 2067 | PLEVWQTYT |
| 2068 | LVTMADHHA |
| 2069 | STENHHFEN |
| 2070 | DALTDFEGC |
| 2071 | EHCDGWRIS |
| 2072 | AQQAVEEVQ |
| 2073 | MITNLFMAG |
| 2074 | NKTDGHYQC |
| 2075 | DGLNEEMWK |
| 2076 | TQLRDMISS |
| 2077 | SEQRTNHYA |
| 2078 | HVTDHQGGI |
| 2079 | HQCCGLVMQ |
| 2080 | AQNGFDKDL |
| 2081 | VFQAFMRSM |
| 2082 | FSCPKDGYA |
| 2083 | NGGMFELTL |
| 2084 | QNDTLEHFY |
| 2085 | DQLQKDFYF |
| 2086 | TSDATCQWI |
| 2087 | ATNENIMAW |
| 2088 | EPAQKAGIS |
| 2089 | QTALRWNWD |
| 2090 | ELEPVVCQW |
| 2091 | NACTHNTFT |
| 2092 | HADANTKTY |
| 2093 | PEYSTDHPN |
| 2094 | YAVTDQEGH |
| 2095 | AIFMHPLTE |
| 2096 | AWQGATHEA |
| 2097 | QTYFMHTSA |
| 2098 | TASHVAEFK |
| 2099 | IHPVQFCKC |
| 2100 | QHMISHDKA |
| 2101 | IYTHCWHEY |
| 2102 | IAEPHYTAA |
| 2103 | TDMIVKEQC |
| 2104 | NNQPPDGAC |
| 2105 | QIEPTPEHT |
| 2106 | EILVYMYFH |
| 2107 | MMCNKIHDE |
| 2108 | MVFTRFDHN |
| 2109 | LWECATCNP |
| 2110 | NVEMGPRAM |
| 2111 | CANMEDTDY |
| 2112 | PDKACQMID |
| 2113 | SHGEKCMSV |
| 2114 | EFTLFDSDR |
| 2115 | ETLFYNAFA |
| 2116 | VTLISHCKC |
| 2117 | LNCCYKEMT |
| 2118 | TCTSCVHDY |
| 2119 | FTCIWFRWA |
| 2120 | MHPSTSVTC |
| 2121 | QKNHFEMQQ |
| 2122 | NDLYKANYN |
| 2123 | TVFQMQMCN |
| 2124 | PEHCEAWPV |
| 2125 | DVEMFFMVG |
| 2126 | MLYWCLQNG |
| 2127 | YTGWLMSDM |
| 2128 | HCVVMQRMY |
| 2129 | LHDVAAENR |
| 2130 | EEEHCAKLG |
| 2131 | HTMYVDTAN |
| 2132 | CLDWLHREY |
| 2133 | PPCETAHTY |
| 2134 | GVHTKMIAW |
| 2135 | HDVCMSYKS |
| 2136 | NLPQPVLMM |
| 2137 | IPGDYVNLS |
| 2138 | TSTVFHNWC |
| 2139 | DKLDINMTL |
| 2140 | CHYQGEHIP |
| 2141 | TYEVNCENA |
| 2142 | NFDTQPKAI |
| 2143 | MCMPADQTL |
| 2144 | AKETWDCVG |
| 2145 | PGPAHVDHI |
| 2146 | ATIVTQHGC |
| 2147 | GMYIMKEHQ |
| 2148 | PTHATTMVQ |
| 2149 | DVYAMVRPC |
| 2150 | ESHPLWHDL |
| 2151 | QSVMASTAN |
| 2152 | NEGVCSYVQ |
| 2153 | HIYSCDQSN |
| 2154 | STTKHENGH |
| 2155 | GQQPYNRME |
| 2156 | ITVTKCSDV |
| 2157 | VADTCCDMM |
| 2158 | DKHLTCQQA |
| 2159 | VEAVLFTAI |
| 2160 | VEPCRWYTD |
| 2161 | CSQSTCKMT |
| 2162 | YIAQPVDMA |
| 2163 | HICDMPKGR |
| 2164 | TIYPRLPLT |
| 2165 | QHSNLTRPC |
| 2166 | EKACCFTHL |
| 2167 | ASEENLMVK |
| 2168 | MKYTAWMDG |
| 2169 | VQYACLYEN |
| 2170 | EATLNDNHC |
| 2171 | ACNLVYADS |
| 2172 | NALNIHNLT |
| 2173 | CWILHMRSA |
| 2174 | GLDVWPWET |
| 2175 | QTAEVKACN |
| 2176 | VHTCMYGMT |
| 2177 | WVMAHNLPS |
| 2178 | PEAGYQSEC |
| 2179 | HAQMMLSGN |
| 2180 | IHFACMVMH |
| 2181 | AWQMQVNGP |
| 2182 | NFQITEAGQ |
| 2183 | CEDSFYKFD |
| 2184 | IMTSQMYPA |
| 2185 | RSELYGMTL |
| 2186 | QTLMVLRNM |
| 2187 | EHLYGDIVY |
| 2188 | MEQQMKMMF |
| 2189 | EFELDTKLC |
| 2190 | TINVQNFPT |
| 2191 | TQVLEEMAW |
| 2192 | TSFSASHWG |
| 2193 | QTIYRFNAA |
| 2194 | VIDPRHDGY |
| 2195 | NEWNQCQAH |
| 2196 | TTVNNHDGW |
| 2197 | CAYQKHGYV |
| 2198 | QTHWQYCRV |
| 2199 | SQARICSYC |
| 2200 | TNNFRMIAS |
| 2201 | TRMQADNMH |
| 2202 | NLDNKNEGP |
| 2203 | RWVGLWCAD |
| 2204 | DRQAMVKTD |
| 2205 | NAQHRSQAP |
| 2206 | EKLMLHQSV |
| 2207 | LVICMQSST |
| 2208 | TVHHGFADP |
| 2209 | KSTCETDIQ |
| 2210 | NQCFHCAWN |
| 2211 | NMPMTQNGH |
| 2212 | LFGMMPQMK |
| 2213 | TIHPFSASR |
| 2214 | FGMQDHCVC |
| 2215 | FGPLLGTIP |
| 2216 | AQGRYASQC |
| 2217 | VIHQEWPCT |
| 2218 | ETPNNEQTH |
| 2219 | QRMYLDHCL |
| 2220 | SSGCWYQLG |
| 2221 | TNEHQMVPW |
| 2222 | RSYVQDFEQ |
| 2223 | EIHVKFRYD |
| 2224 | GIHAMIVSE |
| 2225 | QNEICECYL |
| 2226 | IGVCWNYAD |
| 2227 | DENGYSAPF |
| 2228 | PMIMHQLAG |
| 2229 | DASMWGFHT |
| 2230 | DLSTFNHWY |
| 2231 | QVHQLTESV |
| 2232 | EIVNWLERE |
| 2233 | IFDWANWHY |
| 2234 | CEDILTTAA |
| 2235 | NSYPPGQGA |
| 2236 | TTSPRMMYQ |
| 2237 | TFSFVLDQD |
| 2238 | PLDRINMPF |
| 2239 | TAHSTMWGW |
| 2240 | EIGPDQDTC |
| 2241 | ELHITCLYS |
| 2242 | MNFLFEVSY |
| 2243 | MHGNQHAHK |
| 2244 | EFMNHSNAA |
| 2245 | ETDPTNRID |
| 2246 | GQLVECAAS |
| 2247 | NQHDFIRHF |
| 2248 | WAETGFCME |
| 2249 | VTHQIQRGF |
| 2250 | YRDPWVQCS |
| 2251 | LCNATWADP |
| 2252 | HLWPDTCFQ |
| 2253 | TVPEEYLRP |
| 2254 | GIGCAETAH |
| 2255 | SHDICCKFE |
| 2256 | SHNTCESIE |
| 2257 | ASDPFQMSM |
| 2258 | VFYMGHCCY |
| 2259 | YQAQCAMSG |
| 2260 | FQWCTQLGC |
| 2261 | IMTSWTKIC |
| 2262 | MQNAIARHA |
| 2263 | SVIHFGSYT |
| 2264 | GWQDMMQMP |
| 2265 | LCHADGRCS |
| 2266 | WLMDNTFPW |
| 2267 | LMGYCQKNM |
| 2268 | HQNWDNGAP |
| 2269 | ERFCELHKA |
| 2270 | CSLRTCFGG |
| 2271 | PTCAHNYRT |
| 2272 | LMDHTCKCD |
| 2273 | YTSDAAHQV |
| 2274 | SIQKEDNQH |
| 2275 | YQFPRESVI |
| 2276 | MINGPKCYY |
| 2277 | IANQKGLTV |
| 2278 | SFISWAELC |
| 2279 | RSGYQDWMH |
| 2280 | VHLPDTRNV |
| 2281 | DSGWHHACQ |
| 2282 | FVTTITTHY |
| 2283 | YGDANWAVM |
| 2284 | PVTILADEY |
| 2285 | LNYAGYCRP |
| 2286 | MNIYHENLS |
| 2287 | VTLTCNTNA |
| 2288 | FANASGFWR |
| 2289 | ITNDHNHLW |
| 2290 | DKLHGQREY |
| 2291 | SNTLLCKMV |
| 2292 | QISNVTANN |
| 2293 | GNQWYQCTA |
| 2294 | EIWACYTHG |
| 2295 | DWLQEHNGN |
| 2296 | ETYKSAEWG |
| 2297 | GICMPQEHA |
| 2298 | IHFFNLDHC |
| 2299 | CQELYGIHD |
| 2300 | QHQHVFKDS |
| 2301 | AHCVDMDTW |
| 2302 | NTADKWRLG |
| 2303 | YWHEMLSWP |
| 2304 | SACPSENRE |
| 2305 | PNWQCLEHD |
| 2306 | FLHAVWARP |
| 2307 | TNHWDHRSG |
| 2308 | SHVCRLSAE |
| 2309 | LVCVASSAC |
| 2310 | YVGMIPVHP |
| 2311 | PQSSHIGDT |
| 2312 | GSTIQNRHA |
| 2313 | DHLCFPINE |
| 2314 | TTLALGDYG |
| 2315 | PVHEWKSHQ |
| 2316 | KCASYTEPP |
| 2317 | EWELCNTTT |
| 2318 | EVACNKMMQ |
| 2319 | HTMSTTLVY |
| 2320 | STEACNYGS |
| 2321 | SLMCMEHTY |
| 2322 | TTGIYMCDQ |
| 2323 | FAMVMHDQP |
| 2324 | SRHCCLEFS |
| 2325 | TWNIACGRH |
| 2326 | EVHFSRITC |
| 2327 | FHTNYFWEG |
| 2328 | MTEYKVGFY |
| 2329 | DICDFCRQF |
| 2330 | VTMTVFHPF |
| 2331 | HTELDGCYV |
| 2332 | AWGMACTTW |
| 2333 | WISMTVGAP |
| 2334 | FANYCVAMP |
| 2335 | NILALCEGT |
| 2336 | DRLTHCCEP |
| 2337 | HCDYHVCKG |
| 2338 | HSVIKESCE |
| 2339 | MSMQKMLDN |
| 2340 | VLHQCAIEV |
| 2341 | ANVTMQHQY |
| 2342 | GPVDKAGAN |
| 2343 | KEDSKASNP |
| 2344 | DTPSYKCNW |
| 2345 | LHHFHMKHA |
| 2346 | ATNQVIRHM |
| 2347 | WSASDPYSM |
| 2348 | KEEFVHQQW |
| 2349 | CYQDIQHMV |
| 2350 | DHMEMCSSA |
| 2351 | YPVWVKVQN |
| 2352 | LPQQKNEWY |
| 2353 | TNAEFYMMR |
| 2354 | FWQLIDSAL |
| 2355 | YPVGLYQGI |
| 2356 | CTFPMHGVG |
| 2357 | GDNVCCYPQ |
| 2358 | TVGTMINAY |
| 2359 | DGSERQNAY |
| 2360 | ENFCIYKDS |
| 2361 | EHGMKYQHP |
| 2362 | GATPFEKMN |
| 2363 | HNIWLETGW |
| 2364 | TSHQKFMGS |
| 2365 | LTGSDGQSS |
| 2366 | CEVQCPFDC |
| 2367 | QVWACYLPS |
| 2368 | WFTFFMAGA |
| 2369 | TPKMEICQA |
| 2370 | DNNWMYQAD |
| 2371 | THALSMKGT |
| 2372 | IACLEQATF |
| 2373 | GVNWYINSF |
| 2374 | TIVDWVHMG |
| 2375 | TRLDMAMDA |
| 2376 | PEHPVGYTT |
| 2377 | TEGLFEWGW |
| 2378 | NNPTKTCTT |
| 2379 | CLIQQDVVC |
| 2380 | LWECYYCNS |
| 2381 | PVGIWPSIE |
| 2382 | LREFLTVQT |
| 2383 | LADGQFMGN |
| 2384 | AYQLKVFFN |
| 2385 | WVDNLMRHS |
| 2386 | NAEFEGCVT |
| 2387 | SVTVFWHPG |
| 2388 | MHAPANTQD |
| 2389 | ASYCIHRSY |
| 2390 | TKSEPCLDC |
| 2391 | GEVRMSANY |
| 2392 | EVYSHDSQY |
| 2393 | EQMSNDASY |
| 2394 | YPHAYMVTG |
| 2395 | SAIPFIRHG |
| 2396 | HGGPQVMRS |
| 2397 | NVRECHALV |
| 2398 | TAGMMQYCD |
| 2399 | CEHEMSSYY |
| 2400 | HDNGTNKKM |
| 2401 | MIPAWNVTQ |
| 2402 | AYVSAPRID |
| 2403 | IANQNESSW |
| 2404 | NTFMLELDS |
| 2405 | MITQWMHHR |
| 2406 | ADNVQWSEC |
| 2407 | SITCPSQTC |
| 2408 | FVDPWEWTR |
| 2409 | PCLQIGMQQ |
| 2410 | APCNKTHSW |
| 2411 | DGPSDFKTC |
| 2412 | VNAPPSHVE |
| 2413 | CPNEMVHYK |
| 2414 | VSNEWEASD |
| 2415 | TWNPRCWQP |
| 2416 | FCEQFGSLC |
| 2417 | LQCFLANRE |
| 2418 | RMDMHNNST |
| 2419 | YCNFIVNAM |
| 2420 | PTHTKGNEN |
| 2421 | NNFEQRVGH |
| 2422 | IMGYLWTQN |
| 2423 | YSAWMREFN |
| 2424 | DLFNIIQMD |
| 2425 | FVSNQTKWE |
| 2426 | QCLPLHNHC |
| 2427 | GVGFIPTPY |
| 2428 | DFVSNWDKF |
| 2429 | PQIICMMTV |
| 2430 | RPDHEQEST |
| 2431 | THLFHKEHA |
| 2432 | TGNMAYNCG |
| 2433 | MHDQRMYTW |
| 2434 | VVGWLTHQA |
| 2435 | MILQKNMER |
| 2436 | QVEQCLKED |
| 2437 | LKISPVNER |
| 2438 | AYEFCKNED |
| 2439 | IFEHHNRYP |
| 2440 | DAYTFINGC |
| 2441 | EACLHGFSE |
| 2442 | VMGQMAYQY |
| 2443 | LNSLVVQGK |
| 2444 | HLDPCGWAG |
| 2445 | TYSDKIVNT |
| 2446 | SFGACYQFA |
| 2447 | TQQAFFCVR |
| 2448 | EQMWNHHET |
| 2449 | VKPADANAG |
| 2450 | AIPTFHRAG |
| 2451 | NWIVSAKQQ |
| 2452 | GMYACQMEW |
| 2453 | CGHMSHRSQ |
| 2454 | GVCCNHNQI |
| 2455 | SLGPVNMTS |
| 2456 | TVAGEQMEP |
| 2457 | SLLWAVWLD |
| 2458 | CAVIGVENP |
| 2459 | GVQLIWAGL |
| 2460 | HNYASGWVT |
| 2461 | DHIWRHEAG |
| 2462 | PKLQDVERC |
| 2463 | GWPNQSYIC |
| 2464 | ENENTWHSG |
| 2465 | DKQHTVSAM |
| 2466 | PTGFWPYVH |
| 2467 | GCTMCYMYC |
| 2468 | FTIPQVRYS |
| 2469 | AEEIVYESW |
| 2470 | TTNAPCEIN |
| 2471 | ISDQFGSWI |
| 2472 | IVHGTATAE |
| 2473 | TATIVKQHW |
| 2474 | MICQNMSHT |
| 2475 | LIKPANGEC |
| 2476 | KDFNSELTN |
| 2477 | TPEEPVRGG |
| 2478 | NQMFDQNEY |
| 2479 | YLQVSMQGY |
| 2480 | NHVRNVEQC |
| 2481 | TIMHTQRGA |
| 2482 | FVSQPGYAV |
| 2483 | SMYVMPFWL |
| 2484 | VMILSNHGC |
| 2485 | VWGPFHLTR |
| 2486 | YGYHEDMMC |
| 2487 | TSTAESARD |
| 2488 | NTAHLYGFD |
| 2489 | PFSPKAQVA |
| 2490 | INCYSYNEF |
| 2491 | MKHMPMTDN |
| 2492 | HTHCVLAEH |
| 2493 | MVTTLAGND |
| 2494 | IYVQKHSDW |
| 2495 | PAFLDGVEG |
| 2496 | MTPGRHVLS |
| 2497 | CDQWLQCYQ |
| 2498 | AADSRGVPN |
| 2499 | AWCSFQHIK |
| 2500 | MQMAYYQSH |
| 2501 | QKVCHMTAI |
| 2502 | TNWRTDVNG |
| 2503 | EINEWTIAN |
| 2504 | FTEFYQAHD |
| 2505 | SEKCWPDYQ |
| 2506 | GYTGNDESS |
| 2507 | WFSQNGWAH |
| 2508 | QCALETPIP |
| 2509 | DEILRYSFQ |
| 2510 | KAVITESAC |
| 2511 | ALSAWGMPS |
| 2512 | VIWSVEHAE |
| 2513 | NAHLKMTAS |

In some embodiments, an RPE tissue-tropic VP capsid polypeptide comprises a 581 to 589 region having at least 75% sequence identity to any one of SEQ ID NO: 2014-SEQ ID NO: 2513. In some embodiments, an RPE tissue-tropic VP capsid polypeptide comprises a 581 to 589 region having at least 77.7% sequence identity to any one of SEQ ID NO: 2014-SEQ ID NO: 2513. In some embodiments, an RPE tissue-tropic VP capsid polypeptide comprises a 581 to 589 region having at least 85% sequence identity to any one of SEQ ID NO: 2014-SEQ ID NO: 2513. In some embodiments, an RPE tissue-tropic VP capsid polypeptide comprises a 581 to 589 region having at least 88.8% sequence identity to any one of SEQ ID NO: 2014-SEQ ID NO: 2513. In some embodiments, an RPE tissue-tropic VP capsid polypeptide comprises a 581 to 589 region having a sequence of any one of SEQ ID NO: 2014-SEQ ID NO: 2513. In some embodiments, a retina tissue-tropic VP capsid polypeptide comprises a 581 to 589 region having one or two amino acid substitutions relative to any one of SEQ ID NO: 2014-SEQ ID NO: 2513. In some embodiments, a retina tissue-tropic VP capsid polypeptide comprises a 581 to 589 region having one or two conservative amino acid substitutions relative to any one of SEQ ID NO: 2014-SEQ ID NO: 2513.

### Ocular Tissue-Tropic Sequences Generated by Machine Learning

Additional 581 to 589 regions that confer ocular tissue tropism may be generated using machine learning algorithms trained with sequences identified in ocular tissue in in vivo screens. The machine learning algorithms may be employed to identify patterns in amino acid sequences that favor accumulation in ocular tissue (e.g., retina, RPE, or both) as compared to other tissues (e.g., non-ocular tissues). Alternatively or in addition, the machine learning algorithms may be employed to identify patterns in amino acid sequences that favor accumulation in ocular tissue as compared to a wild type AAV capsid (e.g., comprising a VP capsid polypeptide of SEQ ID NO: 1). These patterns may be used to generate new sequences of 581 to 589 regions that confer ocular tissue tropism to VP capsid polypeptides and rAAVs assembled from the VP capsid polypeptides. In some embodiments, new sequences of 581 to 589 regions may be generated by randomly sampling amino acid residues at each position of 581 to 589 regions that favored ocular tissue tropism in an in vivo screen. The generated sequences may be tested using classifiers (e.g., gradient boosting classifiers or random forests classifiers) to predict ocular tissue-specificity for each 581 to 589 region. The 581 to 589 regions with the highest predicted ocular tissue-specificity may be identified as ocular tissue-tropic sequences.

Disclosed herein are engineered AAV5 VP capsid polypeptides capable of forming an assembled virion that exhibits increased ocular tissue tropism as compared to a wild type AAV VP capsid polypeptide (e.g., a VP1 of SEQ ID NO: 1, a VP2 of SEQ ID NO: 10, or aVP3 of SEQ ID NO: 11), wherein the engineered variant AAV5 VP capsid polypeptide sequence has one or more amino acid substitutions in a 581 to 589 region of the VP capsid polypeptide, corresponding to positions 581 to 589 in SEQ ID NO: 2 (X¹X²X³X⁴X⁵X⁶X⁷X⁸X⁹). In some embodiments, X¹ is selected from A, R, N, D, C, E, Q, G, H, I, L, K, M, F, P, S, T, W, Y, or V. In some embodiments, X² is selected from A, R, N, D, C, E, Q, G, H, I, L, K, M, F, P, S, T, W, Y, or V. In some embodiments, X³ is selected from A, R, N, D, C, E, Q, G, H, I, L, K, M, F, P, S, T, W, Y, or V. In some embodiments, X⁴ is selected from A, R, N, D, C, E, Q, G, H, I, L, K, M, F, P, S, T, W, Y, or V. In some embodiments, X⁵ is selected from A, R, N, D, C, E, Q, G, H, I, L, K, M, F, P, S, T, W, Y, or V. In some embodiments, X⁶ is selected from A, R, N, D, C, E, Q, G, H, I, L, K, M, F, P, S, T, W, Y, or V. In some embodiments, X⁷ is selected from A, R, N, D, C, E, Q, G, H, I, L, K, M, F, P, S, T, W, Y, or V. In some embodiments, X⁸ is selected A, R, N, D, C, E, Q, G, H, I, L, K, M, F, P, S, T, W, Y, or V. In some embodiments, X⁹ is selected from A, R, N, D, C, E, Q, G, H, I, L, K, M, F, P, S, T, W, Y, or V.

In some embodiments, provided herein are AAV5 VP capsid polypeptide having at least one mutation in a 581 to 589 region, wherein said at least one mutation drives increased ocular tissue tropism.

Retina Tissue-Tropic AAV VP Capsid Polypeptides. Described herein are engineered AAV VP capsid polypeptides comprising a variant 581 to 589 region predicted to confer increased retina tissue tropism compared to a wild type AAV VP capsid polypeptide (e.g., a wild type AAV5 capsid polypeptide of SEQ ID NO: 1), generated and selected using machine learning. TABLE 6 provides 581 to 589 region sequences predicted to confer retina tissue tropism or preference.

TABLE 6
Additional Exemplary 581 to 589
Region Sequences for Retinal Targeting

| SEQ ID | |
|---|---|
| NO | 581 to 589 Region |
| 2514 | HKKRARQRP |
| 2515 | HKKKKKQEP |
| 2516 | HKKKARTRP |
| 2517 | HKKRKRVRP |
| 2518 | HKRRARVQP |
| 2519 | AKRRKKRRN |
| 2520 | HKRKKKRRT |
| 2521 | HKRRARYRP |
| 2522 | HKKKGRMRP |
| 2523 | HKRKKKRRP |
| 2524 | HKKRKKQRV |
| 2525 | TKKRKRICP |
| 2526 | HRKRGRVKP |
| 2527 | HKKKARQKT |
| 2528 | HKKRKRLRS |
| 2529 | AKKKKRMRP |
| 2530 | HKKRYKQRP |
| 2531 | HKKRGREQP |
| 2532 | HRRRKKQRP |
| 2533 | HKKKKKQKP |
| 2534 | HDRKKKFRP |
| 2535 | HKKRKRKRP |
| 2536 | HKRKKKLET |
| 2537 | HKKRGRTRW |
| 2538 | HCRRARSYP |
| 2539 | HKKKAKQRP |
| 2540 | HKKRARHKD |
| 2541 | HCKRKKVRS |
| 2542 | HSRRARVQP |
| 2543 | TKKRKRTQW |
| 2544 | HKKRKRMKS |
| 2545 | HKKRKRQKW |
| 2546 | HDKKARSKP |
| 2547 | HKKRKRQRP |
| 2548 | HCRRARMRC |
| 2549 | HKKKKRVAS |
| 2550 | HKRKKRQAY |
| 2551 | APKRKKFRP |
| 2552 | HLRKAKTAP |
| 2553 | AKKKKKFRP |
| 2554 | HKKKGRMKP |
| 2555 | HDRRGKQRP |
| 2556 | HLPRQRVKP |
| 2557 | TDKKKKQCW |
| 2558 | TKKKKKHCP |
| 2559 | SKKKGRERP |
| 2560 | HDKRGKMKP |
| 2561 | KLKRARTQP |
| 2562 | MKKRKRQAW |
| 2563 | HLKRAKFRT |
| 2564 | HKKRKREKP |
| 2565 | MKKRKKQRN |
| 2566 | HDKKKRQRE |
| 2567 | HRRRKKQKC |
| 2568 | HRRRKRLQP |
| 2569 | HGRRAKVRP |
| 2570 | HKKKKRANP |
| 2571 | HKRKKKTRP |
| 2572 | AKRRARTKP |
| 2573 | HKKRLRMRP |
| 2574 | AKKRKRSRP |
| 2575 | HPKRAKAKP |
| 2576 | HKKRKRHKP |
| 2577 | HRRRKKLKN |
| 2578 | HDKKKRVKP |
| 2579 | HQKKKRLQP |
| 2580 | HKKRARFQS |
| 2581 | HKRKKKIKP |
| 2582 | HKKRKRQEP |
| 2583 | HKKRARHRP |
| 2584 | HKKRLKHIW |
| 2585 | HKKKKRMRP |
| 2586 | HCKKKRVRY |
| 2587 | HKKRKRSGN |
| 2588 | HKKKKKARP |
| 2589 | ARKRKKLKP |
| 2590 | SKKKGRQKP |
| 2591 | HKKRDKQRP |
| 2592 | HKKRKRMRY |
| 2593 | HKKRKRHRT |
| 2594 | HPKRKKLAP |
| 2595 | HKRRARQRP |
| 2596 | HKRKKRTRC |
| 2597 | HDKRKRHRW |
| 2598 | QPKRKRVAP |
| 2599 | CDKKKKTRP |
| 2600 | HKRRKKLRP |
| 2601 | HKRKARTRP |
| 2602 | HRKKLKSKP |
| 2603 | HKKKLRVRP |
| 2604 | MDKKGKTCW |
| 2605 | HKKRARVEP |
| 2606 | MKKRGRTPP |
| 2607 | HKRRKRTRN |
| 2608 | AKKKKREKL |
| 2609 | HCRRAKQRE |
| 2610 | HKKKLRMRW |
| 2611 | HPKRKRTRD |
| 2612 | HKKKSKQKP |
| 2613 | HQRRKKKKP |
| 2614 | TKKRKRVKA |
| 2615 | HKKRLRVKP |
| 2616 | HKKKKKVRP |
| 2617 | TKKRKRVRS |
| 2618 | HCRRGKVRC |
| 2619 | HKRRKRMRP |
| 2620 | HKKKKRQAW |
| 2621 | HKKKKRLRP |
| 2622 | HKKKKKRCS |
| 2623 | HDRKKKTKP |
| 2624 | HKKRKRHGP |
| 2625 | HKRRARKRP |
| 2626 | HKKKKKVRC |
| 2627 | HKRKKRQRP |
| 2628 | HKKRKRDRP |
| 2629 | HKKKAKFQP |
| 2630 | HDKRKRTRN |
| 2631 | HKKKKRFNP |
| 2632 | TKKKKRQKW |
| 2633 | ALKKKRQKP |
| 2634 | HKKRLRFKP |
| 2635 | AKKKKRQRP |
| 2636 | HRKRAKVAP |
| 2637 | HCKKKKQIP |
| 2638 | AKKRKRQKW |
| 2639 | HKKKGKHRV |
| 2640 | HKKRDRMCS |
| 2641 | HKKRARLKP |
| 2642 | AKRRKKAKP |
| 2643 | TLKRKRTKP |
| 2644 | HKKRARLAY |
| 2645 | HKKRKRRRW |
| 2646 | HLKKKRKQN |
| 2647 | HKKRKKLRP |
| 2648 | HKRKKKFGP |
| 2649 | EKKKSKMQP |
| 2650 | HCKRAKTRP |
| 2651 | TKKKKRHKN |
| 2652 | HKRRKKERW |
| 2653 | HKRRGKQRP |
| 2654 | HKKRKRTKP |
| 2655 | AKKRAKQRP |
| 2656 | HKKKGRTRP |
| 2657 | HCRRKKVRW |
| 2658 | HKKRKRFKP |
| 2659 | HKKKKRHKP |
| 2660 | HEKRKRFKP |
| 2661 | HKKKKRTRV |
| 2662 | HPRKAKCRD |
| 2663 | HKKRAKQRT |
| 2664 | HKKRARHQT |
| 2665 | HKKRKRHRL |
| 2666 | HRRKAKTRT |
| 2667 | HKKKKRTYW |
| 2668 | HKKRKRLRC |
| 2669 | HKKRKKQRP |
| 2670 | TKKRKRLRP |
| 2671 | HDKRKRVKP |
| 2672 | HDRRKKTAP |
| 2673 | HKRKKKVRP |
| 2674 | AKKKKRQKP |
| 2675 | HLRKKRLRP |
| 2676 | HKKRKRQKP |
| 2677 | HKRKKRQRW |
| 2678 | KDKKKRVRP |
| 2679 | HDRRKKFKH |
| 2680 | TKKKKRMRP |
| 2681 | HKRRKRRGW |
| 2682 | HKRKSRTRP |
| 2683 | HRKRKRVCP |
| 2684 | MKKRKRQRP |
| 2685 | HKKKKKQRP |
| 2686 | HRKKKRVKS |
| 2687 | TKKRARQRA |
| 2688 | MKKKGRVRP |
| 2689 | HRKRGRMRA |
| 2690 | MKKRKRVVP |
| 2691 | HKKRKRLRY |
| 2692 | HCKKKRQRP |
| 2693 | HDRKKKVQP |
| 2694 | HCKRAKTGP |
| 2695 | TPKRKRLRS |
| 2696 | HKKRGRVRP |
| 2697 | TKKRKRQRY |
| 2698 | MRRRGRSRP |
| 2699 | HKKKKKQCP |
| 2700 | HKRKKKDQT |
| 2701 | HCKKARFRP |
| 2702 | HDKRLKQRW |
| 2703 | HLKRKRTRE |
| 2704 | HKKRKRTRW |
| 2705 | HKRKKRFNV |
| 2706 | CDKKLRFFP |
| 2707 | KKRRARQGP |
| 2708 | TKKRSKVRS |
| 2709 | HDKRLRHKE |
| 2710 | AKKKKKVWW |
| 2711 | HKKKKRLRW |
| 2712 | HKKRLKQKN |
| 2713 | HKKRKRLRV |
| 2714 | HKKKKKERP |
| 2715 | HKKRKRSQP |
| 2716 | HKKRARVRP |
| 2717 | HKKKKKFRT |
| 2718 | AKKKGKLRQ |
| 2719 | QKKKGRDRS |
| 2720 | HERKKKLRP |
| 2721 | HKRKKKTKE |
| 2722 | HKKRKKVKP |
| 2723 | HKKRARLRW |
| 2724 | MKKKKRVRP |
| 2725 | HKKRARLRS |
| 2726 | HQRRAKTRP |
| 2727 | HRKKLRQKP |
| 2728 | HDKKKKMMP |
| 2729 | HKRRKRICP |
| 2730 | HRRKKKQKM |
| 2731 | TRKRSRVIN |
| 2732 | HKKRKKRRW |
| 2733 | HKKRKRRVP |
| 2734 | HKKRKRMRP |
| 2735 | TKRRARVRT |
| 2736 | HKKRKRVAP |
| 2737 | HPKRKRHRP |
| 2738 | SKKKKRFAP |
| 2739 | TLRKKRQRP |
| 2740 | KKRKKRKGN |
| 2741 | HKKRKRTRP |
| 2742 | HKKRKRHNP |
| 2743 | HKKKGRHQC |
| 2744 | HKKKKRSRP |
| 2745 | HDKRKKQKP |
| 2746 | HKKRKKQRE |
| 2747 | HKKKGKVKP |
| 2748 | HLKKKKKQP |
| 2749 | HPKKKKLRP |
| 2750 | HRKRAKVRC |
| 2751 | HDKKKRLRP |
| 2752 | TKKRKKDKE |
| 2753 | HKKRARQRY |
| 2754 | MKKRARLRC |
| 2755 | HKKRARFRL |
| 2756 | HKKRGRIRP |
| 2757 | HQKKCKTCP |
| 2758 | HPKRARTRP |
| 2759 | MKKKKRQCN |
| 2760 | HLKKKRWRW |
| 2761 | HKKKKRLKN |
| 2762 | HKKRKRART |
| 2763 | HKKRKKMRP |
| 2764 | HKRRKRRRS |
| 2765 | HQRRKKLRN |
| 2766 | HKRKKRLRN |
| 2767 | HKKRKRMKP |
| 2768 | HKKKKRVPY |
| 2769 | KDKKKKLKP |
| 2770 | AKKKARMKW |
| 2771 | TCKKAKMCP |
| 2772 | HKKKLKMWP |
| 2773 | HDRKKKQIW |
| 2774 | HKKKKRMCP |
| 2775 | HKKKLRQRP |
| 2776 | TKKRKRQRP |
| 2777 | AKKRKRQAP |
| 2778 | KQKKKREWP |
| 2779 | AKKRARQKP |
| 2780 | HCRKKKTRY |
| 2781 | AKKRLKVQP |
| 2782 | HCKKKKQRP |
| 2783 | KKKRKKRRP |
| 2784 | ADRRAKVRP |
| 2785 | HGKKGRVRP |
| 2786 | HKRKAKVRN |
| 2787 | HLKKKRRKT |
| 2788 | HEKRKRERP |
| 2789 | HCRRKKERP |
| 2790 | TKKRKRTAP |
| 2791 | HLRRARLKN |
| 2792 | HKRRKRHRW |
| 2793 | HKRKKRQQQ |
| 2794 | HDRRGKTQP |
| 2795 | TDKRGKQKP |
| 2796 | AKKKKRHKM |
| 2797 | HKRKKKMRP |
| 2798 | HKKRKRLRH |
| 2799 | HCKKARRKP |
| 2800 | HRKRLRQRP |
| 2801 | HKKRGRVKP |
| 2802 | HKKRKKDKS |
| 2803 | MKKRKRHQH |
| 2804 | HKKRKKVKD |
| 2805 | HDKRARQRP |
| 2806 | HDKRKRVWP |
| 2807 | AKKRKKHRP |
| 2808 | TRKKKRVKP |
| 2809 | HKRRKRHRP |
| 2810 | HKKRLRLRP |
| 2811 | HKKKKKLRI |
| 2812 | HRKRLRSRP |
| 2813 | HKRRKKERP |
| 2814 | HKRRKRLRT |
| 2815 | HKKRKRHRN |
| 2816 | HKRRKKLRW |
| 2817 | HKKRSKQRP |
| 2818 | HDKRKRVRP |
| 2819 | HKKKKRVKP |
| 2820 | IKRKKRHYP |
| 2821 | HRKRAKQQP |
| 2822 | HDKRKKMRD |
| 2823 | HDKKLRFRW |
| 2824 | AKKRSRWRC |
| 2825 | HKRRKRQKP |
| 2826 | HERKKRVRP |
| 2827 | HKKRKRTRV |
| 2828 | HKKRLRVRP |
| 2829 | HKRKKKHRP |
| 2830 | HKKRARHRS |
| 2831 | HRKRKRLRP |
| 2832 | HKKRKRTYY |
| 2833 | YKKKKKVCS |
| 2834 | KKKRKRQRW |
| 2835 | HKKRKRVKP |
| 2836 | HKKRKKVRS |
| 2837 | HDKRKKMQP |
| 2838 | TKKKAKQKP |
| 2839 | HKRKKRWRW |
| 2840 | HKKRKRFRP |
| 2841 | QQRKARHRP |
| 2842 | HCRKDKQQP |
| 2843 | HKRKLKWKP |
| 2844 | HKKKKRQKP |
| 2845 | HKKRKRTCP |
| 2846 | TKKKSKTRP |
| 2847 | HKKRGRMRP |
| 2848 | HKRKKKLKC |
| 2849 | HKKKKRFKP |
| 2850 | HRKRARLKP |
| 2851 | HRKKGKQRP |
| 2852 | TRKRKRMRD |
| 2853 | HQKKKRQLP |
| 2854 | CKRKKRFCP |
| 2855 | HPKRKRQYP |
| 2856 | HEKRAKHCP |
| 2857 | HKRRKRQQP |
| 2858 | HKKRKRLND |
| 2859 | HKKRGRQQH |
| 2860 | HKKKSRHRP |
| 2861 | HKKRKRMYP |
| 2862 | HKKRARSRW |
| 2863 | HDKKAKFRP |
| 2864 | HKRRAKQRP |
| 2865 | HKRLLKDRP |
| 2866 | HKKRARQRL |
| 2867 | HKKKKRMKP |
| 2868 | HKRKKRVRW |
| 2869 | HKKKLRMRP |
| 2870 | IKKRARMRP |
| 2871 | HKKRKKLCP |
| 2872 | HKKRKRFCP |
| 2873 | HKKRKKTLA |
| 2874 | TKKRKRVCP |
| 2875 | HPRKKKLRP |
| 2876 | HKKRAKFQP |
| 2877 | ACRKGKQAW |
| 2878 | HPRRKRTKP |
| 2879 | HQRRARLRP |
| 2880 | IKKRKRTRP |
| 2881 | HRKKKKDKS |
| 2882 | HKKRKRQRW |
| 2883 | HKKRKRRRM |
| 2884 | HPKRKKQRP |
| 2885 | HQKRGRCRP |
| 2886 | HPKRARFRP |
| 2887 | HKKKKRQRY |
| 2888 | AKRRAKARP |
| 2889 | HKRKKRQKN |
| 2890 | HKKRAKSQT |
| 2891 | SDKRKRMKT |
| 2892 | HKRKKRKRP |
| 2893 | HKKRKRMRW |
| 2894 | HDKRARTRP |
| 2895 | MDKKKRQKP |
| 2896 | HKKKKRTAD |
| 2897 | HKRKKRLKP |
| 2898 | HKKRKRLKP |
| 2899 | HKKRARTRP |
| 2900 | TKKRARVKP |
| 2901 | HDKRKKLNT |
| 2902 | HKKKGRHRP |
| 2903 | TKKRARFRP |
| 2904 | HKKRKRFNP |
| 2905 | HDKRKRTRP |
| 2906 | TDKRKRFRP |
| 2907 | HKKRKKFQP |
| 2908 | HKKRKRVQC |
| 2909 | HKKKKRQRP |
| 2910 | TRKKKKMKN |
| 2911 | TKKRKRMRS |
| 2912 | HKRRKRTKP |
| 2913 | TRKRARVKP |
| 2914 | TKKKKRMGY |
| 2915 | HKKRGKVRP |
| 2916 | KKKRKRQCV |
| 2917 | HRKRKRTRP |
| 2918 | HDRRAKRAY |
| 2919 | HKKKKRERY |
| 2920 | HKKKKRHRW |
| 2921 | HKRKKKVRY |
| 2922 | HKKKLKTQS |
| 2923 | HPKRKRMCP |
| 2924 | HRKRKRMRP |
| 2925 | HRKRARVKP |
| 2926 | HDKRKRMRP |
| 2927 | HRKKKKVRP |
| 2928 | HCRRAKFLP |
| 2929 | HDRRKRLRP |
| 2930 | HKKRKRFKS |
| 2931 | HCRKKRFRP |
| 2932 | HKKRARQRS |
| 2933 | HKRKKRQKP |
| 2934 | HRRRKRFHW |
| 2935 | HKKKKRVRP |
| 2936 | HKRRKKTRT |
| 2937 | HPKRKRTRP |
| 2938 | HKKKARSRP |
| 2939 | HKKKKKTNN |
| 2940 | HKRRARARP |
| 2941 | HCRKAKQQP |
| 2942 | HPKRAKQIP |
| 2943 | HKKRKRLQP |
| 2944 | HKKKKKGKP |
| 2945 | HKKRKKMCP |
| 2946 | HDKKGRVKP |
| 2947 | HKKRKRVRC |
| 2948 | HKKRKRMDS |
| 2949 | HKRKKRFRS |
| 2950 | HRKKKRHKW |
| 2951 | HQKKKRFRP |
| 2952 | HKRRDRTKP |
| 2953 | HDKKKRMNP |
| 2954 | HKKRSRDRE |
| 2955 | HDKKKKQRC |
| 2956 | TKKKKRFCS |
| 2957 | HEKRGKHRP |
| 2958 | HKKRKRFRW |
| 2959 | PKKRKRMRW |
| 2960 | AKKRSKQQY |
| 2961 | AKKKKRMKW |
| 2962 | AKKKARVIP |
| 2963 | HKKKARQRP |
| 2964 | HKKRKKVNP |
| 2965 | TPKKKRLQN |
| 2966 | HKKRKRLAN |
| 2967 | YKKRKRQRL |
| 2968 | HRRKTRMRS |
| 2969 | HKKRKRQAW |
| 2970 | KKKKKRHRP |
| 2971 | HKKRGKEKP |
| 2972 | AKKRKRQRP |
| 2973 | HKKSAKDRP |
| 2974 | HKKRQRQAP |
| 2975 | HDKKKKFQW |
| 2976 | HKKKKKQRW |
| 2977 | AKKRKRQKP |
| 2978 | HKRRARVKP |
| 2979 | HSRRARMRT |
| 2980 | HKTRARFKP |
| 2981 | AKKRKRLCQ |
| 2982 | ACKRKRMKP |
| 2983 | HCKRARVKP |
| 2984 | HRRRKRGRS |
| 2985 | HKKRARVQP |
| 2986 | HSKRARQGW |
| 2987 | HKKRAKHRC |
| 2988 | HDKKKRHKP |
| 2989 | HKKRARVKD |
| 2990 | HKKRKKVRP |
| 2991 | AKRKKRVKP |
| 2992 | HKRRKKTAY |
| 2993 | HKKKARHRP |
| 2994 | ARKRARQQP |
| 2995 | IKKRGRVRY |
| 2996 | HEKKARVKP |
| 2997 | HKRRKKRRT |
| 2998 | HKRRKRQRP |
| 2999 | TKKRDRHYP |
| 3000 | HDKRAKQRP |
| 3001 | HKKRKRLRW |
| 3002 | HSKKKKFCP |
| 3003 | HKKRKKQRW |
| 3004 | HCKRAKARC |
| 3005 | HKKKKKQQP |
| 3006 | AKRKKKQRP |
| 3007 | HQKRKKDQP |
| 3008 | HKKRKRSKY |
| 3009 | HKRRKKTQP |
| 3010 | HKRRKRTKW |
| 3011 | HDKRARERP |
| 3012 | HKKKKKHKT |
| 3013 | AKKRKRFRP |
| 3014 | KKKRARMRP |
| 3015 | HKKRARHEP |
| 3016 | HKKRAKERP |
| 3017 | HKKRCKSNP |
| 3018 | HKKRARFMP |
| 3019 | HKRSKRQRW |
| 3020 | HLKKKKAKP |
| 3021 | HKKRKKHRP |
| 3022 | HRKRKKQRP |
| 3023 | HKKKKKQKN |
| 3024 | HKRRDRKQP |
| 3025 | HCKKKKQKW |
| 3026 | HKKRKRVRS |
| 3027 | HDKKKKTGN |
| 3028 | EKKRKRHRT |
| 3029 | TKKKKKVRP |
| 3030 | HKRKKKVKP |
| 3031 | HKKRKREQP |
| 3032 | HKKRKKLKW |
| 3033 | HKKRDRTKP |
| 3034 | HKKRKKQNY |
| 3035 | HKRRKRRRH |
| 3036 | ADKKAKVAP |
| 3037 | HCRKKKQRP |
| 3038 | HKKKAKLQP |
| 3039 | HKKKGRFRY |
| 3040 | HDKRKRFKP |
| 3041 | HKRRLRQRP |
| 3042 | HPKRKRERP |
| 3043 | HKKKLKTCP |
| 3044 | HKKKGKQAP |
| 3045 | HKKRKKMRN |
| 3046 | HKKRKKSRN |
| 3047 | TKKRKKVRP |
| 3048 | HDKKARTKP |
| 3049 | HCKRAKQRP |
| 3050 | HRKRARTRP |
| 3051 | ACKRGRLRP |
| 3052 | HKKRKRHRP |
| 3053 | AKKKKRVRP |
| 3054 | TKKRKRVRP |
| 3055 | HKRKARHKN |
| 3056 | HKKKGRLRP |
| 3057 | HQKKGKVKP |
| 3058 | HKKKKKQRT |
| 3059 | AKKRKRRRY |
| 3060 | HKKRGRHKY |
| 3061 | HKKRKKVCP |
| 3062 | HRKKKRQRP |
| 3063 | HDKKSKQRP |
| 3064 | HKRRKRQWP |
| 3065 | AKKRKRLRP |
| 3066 | HKRRKRQPP |
| 3067 | HDKKKKMKV |
| 3068 | HCRRAKTRW |
| 3069 | HCKKKKMRP |
| 3070 | TCRKGKQRP |
| 3071 | HKKRARFKS |
| 3072 | SPKRKRFRS |
| 3073 | ADKKKRHKP |
| 3074 | HDKRARSRP |
| 3075 | HKKKKKQQM |
| 3076 | HKKRGRVQP |
| 3077 | HKRKKKDCQ |
| 3078 | HKKKKKFKP |
| 3079 | HKKKARLQP |
| 3080 | ARKRARVRP |
| 3081 | HQKRGRVYP |
| 3082 | HKKRKRVRY |
| 3083 | HKKKKKLRP |
| 3084 | TKKKKRVRP |
| 3085 | HLKRARQRP |
| 3086 | TKKKKKQRP |
| 3087 | QKKRKKQEW |
| 3088 | HKKRKRRRH |
| 3089 | HKRKKRVRP |
| 3090 | HCKRKRMQS |
| 3091 | HKKRKRTRY |
| 3092 | HKKRKHQKP |
| 3093 | HEKKAKTQT |
| 3094 | HDKKKKTRP |
| 3095 | TKRKGRDKS |
| 3096 | HKKRARVKP |
| 3097 | HCKRKRVRE |
| 3098 | HKKRKRFVL |
| 3099 | TRKRAKVQP |
| 3100 | TKKKKRRIP |
| 3101 | HRKRKRRRP |
| 3102 | HEKKKRQKP |
| 3103 | HKKKGRMRY |
| 3104 | HCRKGRQKY |
| 3105 | HDKRKRTKE |
| 3106 | HQRSAKQCC |
| 3107 | HRKRKRFRP |
| 3108 | HKKKLRVQW |
| 3109 | HKRRARFRP |
| 3110 | HDRRKKQRP |
| 3111 | HKRRKRVAP |
| 3112 | KKKRKRQKP |
| 3113 | HKKKARVKP |
| 3114 | HKRRARMRT |
| 3115 | HKKKAKVKP |
| 3116 | MQKKKRHRP |
| 3117 | HKKKKKVKT |
| 3118 | TDRRLKTIP |
| 3119 | HKRKKRTKW |
| 3120 | HKRKLKQRP |
| 3121 | TKKKKRQRP |
| 3122 | APKRGRFIP |
| 3123 | HRKKLRFKP |
| 3124 | HKKRARVKT |
| 3125 | KKRKKRQRP |
| 3126 | HKKKKRQQP |
| 3127 | HKRRKRSYS |
| 3128 | HCRRAKVKC |
| 3129 | QKKRGRRKW |
| 3130 | HRRKGRQRT |
| 3131 | HKRRKRFRN |
| 3132 | HQRRAKLQY |
| 3133 | HLKRARVRP |
| 3134 | HKKKKRLKP |
| 3135 | HKKKKRTCP |
| 3136 | HDRKKKTRP |
| 3137 | HKKKKKFRA |
| 3138 | HDKRARFQP |
| 3139 | HRKRGRTKS |
| 3140 | HRKRKRHRP |
| 3141 | HEKKGREAP |
| 3142 | HKRKGRVCP |
| 3143 | HKKKGKKRP |
| 3144 | HKKRKKCRN |
| 3145 | HKKRARVRT |
| 3146 | KQKRKRFRS |
| 3147 | HKRKKKDRP |
| 3148 | HRKRSRVRP |
| 3149 | HDKRARVRP |
| 3150 | HKKRLRFRN |
| 3151 | HKKRKRRNW |
| 3152 | HQKRKRTRP |
| 3153 | HKKRGKMKW |
| 3154 | HKKKGKYRN |
| 3155 | HKKKKRMRS |
| 3156 | HKKKKRQCE |
| 3157 | HKKKKRFEP |
| 3158 | HLKRKKSKP |
| 3159 | HCKKCRHRP |
| 3160 | HKKKKRQRE |
| 3161 | HDKRKRLRP |
| 3162 | HPKSARQRP |
| 3163 | HCKRKKLRP |
| 3164 | HDRKKRQRP |
| 3165 | HKRRKREKP |
| 3166 | HKKRKKRSW |
| 3167 | AKKRKRVQT |
| 3168 | HKRRAKQGW |
| 3169 | ADKRKKMRP |
| 3170 | HLKRKRFRP |
| 3171 | TKKRGRTNP |
| 3172 | HKRKKRTRP |
| 3173 | HKKRKRALP |
| 3174 | HKRKKRMRN |
| 3175 | KKKKKKQRW |
| 3176 | HKKRKRVQP |
| 3177 | HKKRKRVCP |
| 3178 | AKRKKKDIP |
| 3179 | HKRKGKMRP |
| 3180 | HKRRKKDQW |
| 3181 | HDKRAKTKP |
| 3182 | HKKKKRVRW |
| 3183 | HKKRKRTKA |
| 3184 | HDRRKRQRW |
| 3185 | HKRKAKERP |
| 3186 | HKKKKRHRP |
| 3187 | HKKKSRVRP |
| 3188 | HKRRKKQKP |
| 3189 | HCRRKRLRP |
| 3190 | HQRRGRLRT |
| 3191 | HKKRKRVRN |
| 3192 | AEKKARTQW |
| 3193 | TKKRKRAKE |
| 3194 | AKKRARQRP |
| 3195 | HRKKKRTAP |
| 3196 | HKKRKRLRP |
| 3197 | PKRKKRQRN |
| 3198 | HKRKKKTRW |
| 3199 | HRKRKRQRW |
| 3200 | TKKRKRRGT |
| 3201 | HKRRKKQGP |
| 3202 | HKKKKKHRW |
| 3203 | HDKKKRERP |
| 3204 | HEKRLKFKW |
| 3205 | IKKRKRMCY |
| 3206 | HKKKKHQRP |
| 3207 | HKKRKRHKC |
| 3208 | HRKKKRQKP |
| 3209 | HDKRKRSRP |
| 3210 | HKKKGRQIP |
| 3211 | HKRRKRTRP |
| 3212 | HRKKKRARP |
| 3213 | HKKRLKRRW |
| 3214 | HCKRKKLAP |
| 3215 | HKKRKKTRW |
| 3216 | HKKRGHQRP |
| 3217 | HKKRKRFQS |
| 3218 | HKKKKRSKP |
| 3219 | HKRKKKVKA |
| 3220 | HKKKKKHRP |
| 3221 | TLKRARVKP |
| 3222 | HKKKKKFQP |
| 3223 | HPRRKRKRP |
| 3224 | AKKRARVRP |
| 3225 | HKKKKRQRT |
| 3226 | HKRKKRTRM |
| 3227 | HRKRSRKRT |
| 3228 | HCRRKKQKP |
| 3229 | HKKRKRQGP |
| 3230 | HKKKLKQRN |
| 3231 | MKKKKRLKW |
| 3232 | HRKKKKQKW |
| 3233 | HKKRKRTKY |
| 3234 | MKKRKKHRC |
| 3235 | TKKRAKMRP |
| 3236 | HRRKKKLRW |
| 3237 | TKKRARSNP |
| 3238 | HKKKTRHRP |
| 3239 | HKRKKRFPP |
| 3240 | HKKKKRAKW |
| 3241 | HKRKKKARP |
| 3242 | HKKRKRHVW |
| 3243 | HDRKKKTRE |
| 3244 | HKKRKRFPP |
| 3245 | HLRKARIEP |
| 3246 | HRRKARQRN |
| 3247 | HCKKKKQRS |
| 3248 | HKKRSRCKP |
| 3249 | HGKKKKQQW |
| 3250 | AKKKLRQRP |
| 3251 | HKKRGRQRP |
| 3252 | AKRKKRKRE |
| 3253 | HKKRKKTRP |
| 3254 | QKRRKRVRP |
| 3255 | HKKRKRQRH |
| 3256 | HKKRGKFCP |
| 3257 | HKKRKRQRS |
| 3258 | HKKKKRIRP |
| 3259 | HKKRAKQRP |
| 3260 | SKKRKRHRP |
| 3261 | IKKRKRVEP |
| 3262 | TKKRKKMRN |
| 3263 | AKKKKKFGS |
| 3264 | TQKKGRVKP |
| 3265 | HDKRGRQRC |
| 3266 | HKRRKRMRW |
| 3267 | HKKRKRTQP |
| 3268 | HKKKKKVKP |
| 3269 | HKKKKKMCW |
| 3270 | HKRRKKQND |
| 3271 | HKKRKRVGP |
| 3272 | YKKRGRMRP |
| 3273 | KPKKKRFRG |
| 3274 | AKKRKKQKS |
| 3275 | HKRRKKWRP |
| 3276 | HRKRKRLGY |
| 3277 | HKRKDKVRT |
| 3278 | HKRKKKHRW |
| 3279 | HKRRKRRRP |
| 3280 | HKKKGRART |
| 3281 | AKKRARWIP |
| 3282 | MKRRKKLRP |
| 3283 | HKRKKKFKW |
| 3284 | TRKRKRVCP |
| 3285 | HQKKAKTEP |
| 3286 | HKRKLKVRP |
| 3287 | IKRRKRMHW |
| 3288 | HKKKKKQKV |
| 3289 | QCKRGRMRP |
| 3290 | HQRKKKHKC |
| 3291 | HKKKAKTRT |
| 3292 | HKKRKRVRE |
| 3293 | TPKRLRMAP |
| 3294 | HCKKARVKP |
| 3295 | TKRRKRFRP |
| 3296 | KDRKKKQRT |
| 3297 | HKRKKRMRP |
| 3298 | HKKKLRQKA |
| 3299 | TDPRGRQKP |
| 3300 | HDKKARQRP |
| 3301 | HKRKKRAWW |
| 3302 | HKKRKRMRC |
| 3303 | HKKRLRLRT |
| 3304 | TKKKNKMGP |
| 3305 | HKRRKRFRY |
| 3306 | HKKRKKTKW |
| 3307 | HDKKARLVP |
| 3308 | HKRRAKTAP |
| 3309 | HORRKKFNN |
| 3310 | HKKKKRLCW |
| 3311 | HKKRKRFRC |
| 3312 | EKKKKRVKP |
| 3313 | HKKRKKQKP |
| 3314 | TKKKKKQKP |
| 3315 | HKKRKKQQN |
| 3316 | HKKRGKAKW |
| 3317 | AKKKGRHRP |
| 3318 | HKKRKRRRP |
| 3319 | AKKRARTRW |
| 3320 | AKKKKRQCN |
| 3321 | HRKRKRTAY |
| 3322 | HKRKKRFIP |
| 3323 | TKKRKRTKP |
| 3324 | KDKKAKTQP |
| 3325 | HKKKKRHRC |
| 3326 | HRKKKKTWT |
| 3327 | AKRKGRLFP |
| 3328 | HKKKKHTNP |
| 3329 | HKKRLRVRW |
| 3330 | HKKRKRQRL |
| 3331 | HKRRARQRY |
| 3332 | HCKKARSKP |
| 3333 | HKKKKKRNH |
| 3334 | HKKRSRLKP |
| 3335 | HDKRAKQQP |
| 3336 | HRKRGRLNP |
| 3337 | HKKKAKARP |
| 3338 | HKKRARMKP |
| 3339 | HKKRGKVEY |
| 3340 | HRKRQRARP |
| 3341 | HPKKKKQKP |
| 3342 | TVKRARVAT |
| 3343 | HDRKLRQRP |
| 3344 | HEKRKRMAP |
| 3345 | HKKRSRMRP |
| 3346 | HDKKKKLRP |
| 3347 | HKRKGRGRP |
| 3348 | HKKKAKVAS |
| 3349 | TKKRGRQCP |
| 3350 | HCKKKRQKS |
| 3351 | AKKRAKQKS |
| 3352 | HGKRKKVHT |
| 3353 | HRKKKKVPP |
| 3354 | SCKRGRTRP |
| 3355 | HPKKKRVRP |
| 3356 | HKKRKRFWH |
| 3357 | HKKKKKFRP |
| 3358 | HQKRKRVRA |
| 3359 | HKKKARARP |
| 3360 | HKKRKRFGP |
| 3361 | HRKRKKMRP |
| 3362 | ADRKKRMRN |
| 3363 | AKRRKRHAP |
| 3364 | HKKRKKTNC |
| 3365 | HQKRDKQRT |
| 3366 | HRKRKRQRC |
| 3367 | HQKRKRVKP |
| 3368 | AKKRKRVRP |
| 3369 | HKRKKRHKC |
| 3370 | AKKRSRVHP |
| 3371 | HRKRKRHRW |
| 3372 | HKKRKRFRT |
| 3373 | TKKRKRVQP |
| 3374 | AKKRKKVKP |
| 3375 | HKKRGRHRS |
| 3376 | HRKKKRLRW |
| 3377 | SKRKKRFKD |
| 3378 | HDKRGRTKP |
| 3379 | HERKKRERP |
| 3380 | AKKRKRVAP |
| 3381 | HKKRLHVRE |
| 3382 | HKKRGRLRP |
| 3383 | HPKRKRVKP |
| 3384 | HKKRSRQRP |
| 3385 | HRKKKRTRP |
| 3386 | AKKRLRHRS |
| 3387 | HQKKKRTRW |
| 3388 | HKKRKRGKY |
| 3389 | HDKRARFRY |
| 3390 | TKCRKRVRP |
| 3391 | TRKKKRVCP |
| 3392 | HKKRKRVWY |
| 3393 | HQKKLRSKP |
| 3394 | HKKRLRVRN |
| 3395 | HLRKAKTKP |
| 3396 | HKRKKRHKP |
| 3397 | HKRKARQRE |
| 3398 | HKKRARHKP |
| 3399 | HKRKAKQAV |
| 3400 | HDKRKRTQP |
| 3401 | HQKKKRVKP |
| 3402 | HPKRLRSRA |
| 3403 | HCKRGRVRP |
| 3404 | HLRKKRLKP |
| 3405 | MKKRKRVRP |
| 3406 | KKKKLKFQY |
| 3407 | HKRKGKQRP |
| 3408 | HDKKKKQRP |
| 3409 | TKKRARQRP |
| 3410 | HKRKKKLQL |
| 3411 | MKKRKRRRP |
| 3412 | HKKRGKDCW |
| 3413 | PKRRGRTAP |
| 3414 | HECRGRFRP |
| 3415 | HRRRKRARP |
| 3416 | AKKRKRHRP |
| 3417 | HDKRGRHRN |
| 3418 | AKRRARVKP |
| 3419 | HCRKKKQRY |
| 3420 | HKKKKRFRH |
| 3421 | HKKRKRHRS |
| 3422 | HKKRSRVRP |
| 3423 | HKKRGRSRT |
| 3424 | HKKKKRTKW |
| 3425 | MKKKKRFRP |
| 3426 | ADKKKKMRP |
| 3427 | HRKRKRFKS |
| 3428 | HKTRSRQKY |
| 3429 | IKKKKRMRP |
| 3430 | TKKRKRFKW |
| 3431 | TKKKAKLKC |
| 3432 | SDKRGRMCP |
| 3433 | HKKRAKVKP |
| 3434 | HKKKGRQRI |
| 3435 | HLKKGKLRP |
| 3436 | HKKRAKFKH |
| 3437 | KKKKKRTRI |
| 3438 | HKKRAKQKP |
| 3439 | YKKKKKMKN |
| 3440 | HKKKKRKRP |
| 3441 | HKKRKKDKP |
| 3442 | HDRRKRLKP |
| 3443 | AKKRSRVRP |
| 3444 | HKKKKRFKW |
| 3445 | HKKRKRFRN |
| 3446 | AKKKKRVCS |
| 3447 | HKRRKRFIP |
| 3448 | MEKRKRLRP |
| 3449 | HKRKAKDRW |
| 3450 | HRRRARMEP |
| 3451 | HKKRLREKT |
| 3452 | KPKKKRMKN |
| 3453 | QKRRKRRRC |
| 3454 | HRKKARVRW |
| 3455 | HKRRARLRP |
| 3456 | HKKRKRFWT |
| 3457 | HKKKKRLAP |
| 3458 | HRKKKKQRN |
| 3459 | HKKRKKARP |
| 3460 | HKKRLRTKP |
| 3461 | HEKRKREQP |
| 3462 | HCKRKRVRW |
| 3463 | HKKKKKSRP |
| 3464 | TKKKDRHKP |
| 3465 | HKRKLRFRP |
| 3466 | HEKRKKARD |
| 3467 | AKKKKRFRP |
| 3468 | HQRKARMKT |
| 3469 | AKKRKRQKN |
| 3470 | AKRKLRMRW |
| 3471 | MKKRKKFKN |
| 3472 | HKRKARLRS |
| 3473 | HDKRKKQQP |
| 3474 | HKKKKRRCM |
| 3475 | HKKRGRGRW |
| 3476 | HPKRKRTCP |
| 3477 | ACKKKKQQP |
| 3478 | PKKRGRTQP |
| 3479 | HKKRKKERP |
| 3480 | HKRRKRRRW |
| 3481 | SKKRKRMRP |
| 3482 | HLKKGRHRP |
| 3483 | TKKKDKHKC |
| 3484 | AQKKAKARP |
| 3485 | AKKRSKQRP |
| 3486 | HCRKARQQP |
| 3487 | HKRRSRLKW |
| 3488 | HDRRKRSAA |
| 3489 | HKRRTRVRP |
| 3490 | EKRRKRVQP |
| 3491 | HDRKKKTQC |
| 3492 | SKRRKRFGP |
| 3493 | HQKKLKERP |
| 3494 | HLKRKRVKP |
| 3495 | HPKRKKQQW |
| 3496 | HKRKKKTRL |
| 3497 | HPKRLKVRP |
| 3498 | HKKKKRTQW |
| 3499 | HRKRKRTGP |
| 3500 | HKKRKRSKT |
| 3501 | HRKRKRMRC |
| 3502 | HKKRKRQAA |
| 3503 | HKRRARHRT |
| 3504 | TKKRKKSRN |
| 3505 | QKKRKRQRY |
| 3506 | HKKKARQKN |
| 3507 | HKKRKRECS |
| 3508 | TKKRKRQIC |
| 3509 | HDKRKRIKP |
| 3510 | HDRRKKMRP |
| 3511 | TKRRARTKY |
| 3512 | HKKRKKFRP |
| 3513 | HKKRARLRP |
| 3514 | AKKRARMRP |
| 3515 | QRKRARVRN |
| 3516 | HPKRKKTKP |
| 3517 | TKKKKRFKW |
| 3518 | HKKRKKHCP |
| 3519 | HQKKKRKCP |
| 3520 | HKRRKKTRS |
| 3521 | HKKKKKTRT |
| 3522 | HDKRKRLQP |
| 3523 | CKRKKRTCP |
| 3524 | HKKKLQTKN |
| 3525 | KPKKKRVNH |
| 3526 | TKKKKRTRT |
| 3527 | HGKKKRVKP |
| 3528 | TKKKKRTRP |
| 3529 | HKKKKRTRP |
| 3530 | HDKRKKQRE |
| 3531 | HKKKKRLRS |
| 3532 | HKKRAKLRP |
| 3533 | HKKKAKDRP |
| 3534 | KKKKKRLRP |
| 3535 | MKRKKRFRC |
| 3536 | HCKKAKQRP |
| 3537 | TRPRKRTKP |
| 3538 | HDRKGKQRP |
| 3539 | HKRRAKFRT |
| 3540 | HRKRGRLRP |
| 3541 | HKKRKKQQP |
| 3542 | HKKKKRQRS |
| 3543 | HDKKARVKP |
| 3544 | HEKKGRMRP |
| 3545 | KKKRKRHRP |
| 3546 | KKRKKRVRP |
| 3547 | AKKRLRHRP |
| 3548 | HKRKGKFRP |
| 3549 | HDRKAKVRP |
| 3550 | HQRKKKMQW |
| 3551 | HKKRSRQKS |
| 3552 | HKKRGRFRE |
| 3553 | HKKKKKARW |
| 3554 | HCKRARTAH |
| 3555 | HKKRQRMRC |
| 3556 | HKKRARQAP |
| 3557 | HKRRAKVIP |
| 3558 | HCRKDKTRW |
| 3559 | TKKRARLRP |
| 3560 | HRRKKKLAS |
| 3561 | HKKRAKQKY |
| 3562 | AKKRKKQHP |
| 3563 | HPKRKRQKP |
| 3564 | HKRKARVRP |
| 3565 | HKKKKRARP |
| 3566 | HKKKKKMRC |
| 3567 | HKKKAKTRP |
| 3568 | HQKRKKQRP |
| 3569 | HRKKLRCRW |
| 3570 | IKKKKKMRS |
| 3571 | HKKRSRLRP |
| 3572 | HRRKKRVGP |
| 3573 | HKRRDRQRS |
| 3574 | HQKRGRVRT |
| 3575 | HKKRGKMRP |

In some embodiments, a retina tissue-tropic VP capsid polypeptide comprises a 581 to 589 region having at least 75% sequence identity to any one of SEQ ID NO: 2514-SEQ ID NO: 3575. In some embodiments, a retina tissue-tropic VP capsid polypeptide comprises a 581 to 589 region having at least 77.7% sequence identity to any one of SEQ ID NO: 2514-SEQ ID NO: 3575. In some embodiments, a retina tissue-tropic VP capsid polypeptide comprises a 581 to 589 region having at least 85% sequence identity to any one of SEQ ID NO: 2514-SEQ ID NO: 3575. In some embodiments, a retina tissue-tropic VP capsid polypeptide comprises a 581 to 589 region having at least 88.8% sequence identity to any one of SEQ ID NO: 2514-SEQ ID NO: 3575. In some embodiments, a retina tissue-tropic VP capsid polypeptide comprises a 581 to 589 region having a sequence of any one of SEQ ID NO: 2514-SEQ ID NO: 3575. In some embodiments, a retina tissue-tropic VP capsid polypeptide comprises a 581 to 589 region having one or two amino acid substitutions relative to any one of SEQ ID NO: 2514-SEQ ID NO: 3575. In some embodiments, a retina tissue-tropic VP capsid polypeptide comprises a 581 to 589 region having one or two conservative amino acid substitutions relative to any one of SEQ ID NO: 2514-SEQ ID NO: 3575.

TABLE 7 provides additional 581 to 589 region sequences predicted to confer retina tissue tropism or preference, generated and selected using machine learning.

TABLE 7
Additional Exemplary 581 to 589
Region Sequences for Retinal Targeting

| SEQ ID | |
|---|---|
| NO | 581 to 589 Region |
| 3576 | KGRRKKVSG |
| 3577 | AQKKVKKFG |
| 3578 | HKKRRKVWH |
| 3579 | KSKIKKYAG |
| 3580 | QNKKKKWSW |
| 3581 | HKKKLKWVS |
| 3582 | KKKKPVTWG |
| 3583 | KVKTKKKWA |
| 3584 | KTKKIKSHG |
| 3585 | KRKKAARKP |
| 3586 | KQKIKKSSG |
| 3587 | KKKKTRHQR |
| 3588 | KVKKKVCGG |
| 3589 | KKRPKRRQC |
| 3590 | MAKRKKWRP |
| 3591 | KTKKKHTSD |
| 3592 | RNKKKKRCW |
| 3593 | CKKKKRKGC |
| 3594 | KMRKKKCEQ |
| 3595 | KKKFVKMTH |
| 3596 | KTKAFKKLG |
| 3597 | KVKRKRKAG |
| 3598 | KVKTCKKPQ |
| 3599 | MKKLKKLWC |
| 3600 | KWKKKKGIF |
| 3601 | FKKKGKAHA |
| 3602 | LKKMKKQAC |
| 3603 | KTRKTKWSP |
| 3604 | KAKWYKKGN |
| 3605 | KRKRRRKKS |
| 3606 | KKKVWIKSQ |
| 3607 | KYKNKKWNS |
| 3608 | NVKRYRHAQ |
| 3609 | RTKKRRSCG |
| 3610 | IPKKKRKWC |
| 3611 | KGKLKKHCT |
| 3612 | HRKKKRQWS |
| 3613 | KPKKAKQQQ |
| 3614 | HKKKMGRGH |
| 3615 | KKKTRKVQE |
| 3616 | NKKHKKRGM |
| 3617 | KKKSKITNH |
| 3618 | LCKKKKRFQ |
| 3619 | KAKRQRWRP |
| 3620 | QYKKKKRVS |
| 3621 | TKKRRRRMT |
| 3622 | KARKLKIQG |
| 3623 | KTHRKKKFM |
| 3624 | TKRKKKIGP |
| 3625 | SKKRNKVCT |
| 3626 | KSKFHKQKA |
| 3627 | KKLKAKVCM |
| 3628 | KLRKKRTRG |
| 3629 | HKKRRKSHH |
| 3630 | RKKKRYKIV |
| 3631 | KKKNKLHVQ |
| 3632 | KSKWVKKWH |
| 3633 | RKRKKARTP |
| 3634 | KKKSKHMQA |
| 3635 | KRKRAKVNC |
| 3636 | NWKKKKVCT |
| 3637 | IKKKKKHND |
| 3638 | KHKMKKHVG |
| 3639 | KKKKMHPCT |
| 3640 | KVKGRKIFK |
| 3641 | KKKKLKSWF |
| 3642 | NRKRRRKKM |
| 3643 | KAKRPKSKA |
| 3644 | KKARKKFVA |
| 3645 | KKRKIPRKG |
| 3646 | GRKKFKIKN |
| 3647 | KVKVKKGYW |
| 3648 | KVRKEKTKH |
| 3649 | MKKKKRLLV |
| 3650 | KHRKSKTRT |
| 3651 | YKKRRKRWA |
| 3652 | KKRGKKFSP |
| 3653 | KLKKRGKGG |
| 3654 | ATKRKKKAL |
| 3655 | KKKKAHATY |
| 3656 | KHKKKHYHN |
| 3657 | HRKRTRYGC |
| 3658 | KMKRKKSNQ |
| 3659 | KRKRQKQYQ |
| 3660 | AHKKKKHLT |
| 3661 | KSRKSKGKG |
| 3662 | QTKRRKKGC |
| 3663 | KGKKFKKGV |
| 3664 | KAKQKWVKF |
| 3665 | RVKRCRRCA |
| 3666 | RAKRPRRQW |
| 3667 | KQKKKQYPQ |
| 3668 | KVKRWRGQQ |
| 3669 | CKRRRKRYI |
| 3670 | KKKYRKRRG |
| 3671 | KRRKKGNAT |
| 3672 | SKKRLKSHG |
| 3673 | KKKRKSHNS |
| 3674 | KKKDQKACV |
| 3675 | LRKKKKQTG |
| 3676 | TRKRKKKVK |
| 3677 | VKKKQKSHE |
| 3678 | KKKKFKNVE |
| 3679 | RAKQKKKQS |
| 3680 | KMRKSKVCP |
| 3681 | ARKRKKSCC |
| 3682 | MKHKKKWCH |
| 3683 | QHKKKKRYA |
| 3684 | KRKMIKKCM |
| 3685 | KKMKKRQTG |
| 3686 | KNKKKKKMC |
| 3687 | KCKRKRRRG |
| 3688 | AKKKRKVIM |
| 3689 | HRRKQRRYQ |
| 3690 | KKRKQKRGM |
| 3691 | KKARKTKSH |
| 3692 | KKRKFNRAA |
| 3693 | KRKKSRSYG |
| 3694 | MKKVKKKTG |
| 3695 | KGRRQKRAA |
| 3696 | KNKKKREAT |
| 3697 | YKKMKRKQV |
| 3698 | KRKKKPNDQ |
| 3699 | NKRKKRGGP |
| 3700 | TARKKKCKM |
| 3701 | STKRKKKGN |
| 3702 | HKKRGRSMH |
| 3703 | KKRKYHCDW |
| 3704 | KCKSKKKIA |
| 3705 | YKRKWKHCE |
| 3706 | TIKKFKRKH |
| 3707 | RAKRKRKNM |
| 3708 | AKKRKKDWS |
| 3709 | KARKARRFF |
| 3710 | KKKKHLSNM |
| 3711 | KKKALKRPM |
| 3712 | KGKRQKKQE |
| 3713 | CHKNFKKKG |
| 3714 | KKKTNKCTN |
| 3715 | RTKKRKCFA |
| 3716 | RIRKRKQRQ |
| 3717 | KKRIMKKSH |
| 3718 | APRKKKEKG |
| 3719 | KTKKFKWRV |
| 3720 | QKRKEKKCC |
| 3721 | LKKRKRVAA |
| 3722 | RVKRKRYWQ |
| 3723 | CKKLWKKHH |
| 3724 | KKKWIRRKD |
| 3725 | KLRKMKKTP |
| 3726 | RKKKKAWAT |
| 3727 | KVKKWGWKT |
| 3728 | IKKKIKIAG |
| 3729 | RVKRARHYS |
| 3730 | KKRRCQRGQ |
| 3731 | KKKKWWQCA |
| 3732 | KAKFTKKYQ |
| 3733 | RRKKAKHCS |
| 3734 | KRKKYKWQL |
| 3735 | TKAKKKAPV |
| 3736 | IRRKKKNGS |
| 3737 | HKKRKRCNN |
| 3738 | QSKRKKRKW |
| 3739 | RHRKKRQYP |
| 3740 | KQRKKKTAN |
| 3741 | KARAKKLKA |
| 3742 | KKRRYRHEY |
| 3743 | KRKRRHHTH |
| 3744 | KKKGYKCMW |
| 3745 | KVKKAKQIF |
| 3746 | KKKVKMHIG |
| 3747 | VYKNKKVKS |
| 3748 | KKHKTKAHH |
| 3749 | MAKKKKWQI |
| 3750 | RKRKIRRNC |
| 3751 | KKKKCRLQW |
| 3752 | KKKRNKAWW |
| 3753 | KKKRKDRAY |
| 3754 | CKKTKKYFT |
| 3755 | MKKKQKNTY |
| 3756 | KKKGKGINS |
| 3757 | CKKGRKKWM |
| 3758 | KKRRYKCAH |
| 3759 | QAKKKKKQG |
| 3760 | KKKRKARMD |
| 3761 | RMKRRRHSS |
| 3762 | TNKPKKCKG |
| 3763 | PKKRKFVKG |
| 3764 | KKRKGTGGW |
| 3765 | NKKVQKKPC |
| 3766 | TGKKKKWQP |
| 3767 | QGKKKKVNG |
| 3768 | KYRKWKRTM |
| 3769 | KRRRARQSS |
| 3770 | KSKKNSYKN |
| 3771 | KSKVTKTKG |
| 3772 | KSRKRRTQW |
| 3773 | KKKQNKWKW |
| 3774 | KNKFTKMKS |
| 3775 | MKKMKKCSG |
| 3776 | KMKRKKGQS |
| 3777 | KKLRKKSCG |
| 3778 | KFKKKHIME |
| 3779 | KVKWNKKCT |
| 3780 | KAKKKVRLC |
| 3781 | KKKHTTSKG |
| 3782 | HKKRRARAH |
| 3783 | NKKRKRGHY |
| 3784 | KKKKRFHFN |
| 3785 | KCKRKHVAS |
| 3786 | HKKKVKSHN |
| 3787 | VVKWKKCKT |
| 3788 | KSKNKKGCN |
| 3789 | KCKKKQNHV |
| 3790 | KGKRKRKHS |
| 3791 | KIRKHKKDN |
| 3792 | NKRKRKYKG |
| 3793 | KVKWKQKVG |
| 3794 | KMKHKKHSA |
| 3795 | AKKAQKQKQ |
| 3796 | KKKAKSYDN |
| 3797 | KSKTFKYGG |
| 3798 | KTKPKKCGG |
| 3799 | KAKRKTFVS |
| 3800 | KVRKKRQHA |
| 3801 | KTRKYKIYQ |
| 3802 | KKKKSMRHR |
| 3803 | QKKKKSCHG |
| 3804 | HKGKKKALG |
| 3805 | KVKMRKKNT |
| 3806 | KRKNKKSFW |
| 3807 | YVKRRKKKN |
| 3808 | KKKCKWGMM |
| 3809 | GKKGNKCKQ |
| 3810 | VKKKIKVST |
| 3811 | KWKRKCKGQ |
| 3812 | KKQKKRTYT |
| 3813 | KTRAKKKTT |
| 3814 | KKKKYHHDT |
| 3815 | WKKRKKAGF |
| 3816 | CCKTKKQKG |
| 3817 | KARRRKRYP |
| 3818 | KKKKTRVIE |
| 3819 | QQKAKKCKG |
| 3820 | KCKKKIRWT |
| 3821 | KKKLKRRAN |
| 3822 | VAKRRRKCC |
| 3823 | KQKKSKYTH |
| 3824 | KKKMRKREH |
| 3825 | KRKKKWKAH |
| 3826 | KRMKKKRAY |
| 3827 | SRKKKKKMS |
| 3828 | KKKQGRFKE |
| 3829 | GIRKKKKTT |
| 3830 | LVKPKKKAQ |
| 3831 | KIKGRKKHC |
| 3832 | KGKCKKTHH |
| 3833 | KLKKRKSVN |
| 3834 | KARKSKRAT |
| 3835 | KPRKKKGSE |
| 3836 | KRKRMRNMH |
| 3837 | HKRKKPPKE |
| 3838 | KKKSKYSGV |
| 3839 | RSRKKRRGM |
| 3840 | KRKRHKHQS |
| 3841 | HKRKGKSKW |
| 3842 | KKRKLKWFW |
| 3843 | KQKVKKENC |
| 3844 | GKKKGKRMN |
| 3845 | KHKKCIKCA |
| 3846 | KTRKRRAYC |
| 3847 | SKKTYKKAY |
| 3848 | TKKKGKQYH |
| 3849 | KIKMKKLQF |
| 3850 | KSRMKKLKT |
| 3851 | KKRRRWRYM |
| 3852 | SKKRVKSSG |
| 3853 | KNKRKKNHD |
| 3854 | MKHKKKQQN |
| 3855 | RTKKRRRQH |
| 3856 | KQKIKKMGF |
| 3857 | QSRKKKRNS |
| 3858 | KGKKRKQMH |
| 3859 | YNKKKKHSG |
| 3860 | KGKAKKRRH |
| 3861 | KKAKGKAYP |
| 3862 | MIKIKKKGT |
| 3863 | KKKKVYRYS |
| 3864 | KKKKTVPAW |
| 3865 | CFKKRKKGV |
| 3866 | KCKRTKKRN |
| 3867 | KVKCKKHSG |
| 3868 | KVKKMKSMW |
| 3869 | KVRRYKRLS |
| 3870 | NKQRKKKQC |
| 3871 | KAKTKRHKD |
| 3872 | KVKRMKMTA |
| 3873 | MKKKKVRYH |
| 3874 | KSKCKKGRA |
| 3875 | TCKWKKAKH |
| 3876 | KVKRVRKYW |
| 3877 | FKKKTKCMG |
| 3878 | KTKKKTTMF |
| 3879 | MRRKKKQVY |
| 3880 | KKKRKRRES |
| 3881 | RKKKKKAGD |
| 3882 | CGRKKKRCD |
| 3883 | KVKKRKMYM |
| 3884 | KKKRKKIGL |
| 3885 | HKKRRRYPY |
| 3886 | KKKKAHCSC |
| 3887 | KVRKTKACI |
| 3888 | SQKKKRVKH |
| 3889 | LMRKKKQVP |
| 3890 | KKKAKSTDC |
| 3891 | CRKKKKAHV |
| 3892 | KKKCRKHQY |
| 3893 | KARKKFKTN |
| 3894 | KGRKKRHPG |
| 3895 | KVKKSKHMM |
| 3896 | KRKQKFAKH |
| 3897 | KNRMKKKHC |
| 3898 | LCKSFKKKF |
| 3899 | KAKKRRQFH |
| 3900 | KLKRRRRHS |
| 3901 | EKKKKRRVC |
| 3902 | KASKKKRYH |
| 3903 | GIKKKKSVM |
| 3904 | TKKRKRVKM |
| 3905 | VCKKKKMKH |
| 3906 | HIKKKKGFG |
| 3907 | KKRQKKMCA |
| 3908 | CKKKMKTGW |
| 3909 | HKRRRFTGY |
| 3910 | KRRKHKKMM |
| 3911 | QCKKQKQKS |
| 3912 | IKRRKKCMH |
| 3913 | KNKAQKKPA |
| 3914 | KWKKMKLFM |
| 3915 | KRRRRARGW |
| 3916 | KVKGRKKYP |
| 3917 | NKKHKKCHS |
| 3918 | KKKRVGQVA |
| 3919 | KAKPKKNHC |
| 3920 | TMKRKKHQC |
| 3921 | KRKGVKKRQ |
| 3922 | KRKKMTKSL |
| 3923 | KKRRQKTVQ |
| 3924 | RVRRRKSTG |
| 3925 | ATKKKRKNM |
| 3926 | KKKFKNWTY |
| 3927 | RTKRYRWRC |
| 3928 | RTKAKKTKS |
| 3929 | KKKMGKKCN |
| 3930 | KAKRFKLYT |
| 3931 | KKKKCWMEQ |
| 3932 | KGKKNKMIY |
| 3933 | KKKRVSWSC |
| 3934 | RHKKKKDAH |
| 3935 | KKKWKASQC |
| 3936 | KSKKFKLSY |
| 3937 | KTRKVKWCM |
| 3938 | RKKKKRRSM |
| 3939 | RFKKKKRFA |
| 3940 | KFKKKCYAW |
| 3941 | KKKKSRTRH |
| 3942 | HKRKKSWHL |
| 3943 | KKKRNRRSA |
| 3944 | RKKRVRNSH |
| 3945 | CKRKKKHTL |
| 3946 | HKRKQRQRC |
| 3947 | AKKKKKHKP |
| 3948 | KWKMKKWMP |
| 3949 | RRKRRHSAT |
| 3950 | RIKRKRWSS |
| 3951 | HKKYCKKTS |
| 3952 | RVRRRKWCE |
| 3953 | KSKRCKFKQ |
| 3954 | KKKKMMCNH |
| 3955 | KVKKVRKYH |
| 3956 | KKKKVFKTH |
| 3957 | KTKKKSYDM |
| 3958 | KTKLKKMFS |
| 3959 | TKKTMKKAN |
| 3960 | ARKKKKKRG |
| 3961 | KWRKAKAKY |
| 3962 | KKRKQKSTH |
| 3963 | KRKRKITSD |
| 3964 | RAKRIRIRC |
| 3965 | KTKTLKTKF |
| 3966 | QARKKKRLG |
| 3967 | KTKKKLMGN |
| 3968 | IKKRRKLHP |
| 3969 | KKKKKFRCS |
| 3970 | KQKFGKKAG |
| 3971 | KKRKIVCGY |
| 3972 | KKRGKKKGH |
| 3973 | KKKGQKGYA |
| 3974 | LRRRKKNRY |
| 3975 | KVKKIKNRD |
| 3976 | KRKRFKNDK |
| 3977 | KRRKTNRQP |
| 3978 | KTKRYRFHN |
| 3979 | KARRKRCQC |
| 3980 | KTKKRRSSN |
| 3981 | QVKKKKRLM |
| 3982 | KVKRKKIGW |
| 3983 | RSRKRKSYV |
| 3984 | KRRKFKRSD |
| 3985 | MKMKKKRGH |
| 3986 | KKKKFIKSL |
| 3987 | KVKRRKRRM |
| 3988 | KVKKGQQKC |
| 3989 | VKKSVKKIC |
| 3990 | KKKAKHGWE |
| 3991 | KTKRKRAYW |
| 3992 | KKFKCKNSW |
| 3993 | RRKRFRFYS |
| 3994 | TRRKGKRCN |
| 3995 | KKKKMYHSS |
| 3996 | KVRKKKDHC |
| 3997 | SKKRYKRGS |
| 3998 | KNKKCKFSN |
| 3999 | HKRKSQHHA |
| 4000 | KVKKKRLSA |
| 4001 | KAKRKRRHN |
| 4002 | RAKKKKRAG |
| 4003 | KRRRQRRHH |
| 4004 | KTKKKAKCV |
| 4005 | KVKKYKAHY |
| 4006 | KLKKKRRYF |
| 4007 | RVKKKRQAE |
| 4008 | KNKKVVKHH |
| 4009 | KFKKMRKAT |
| 4010 | KKKKRSYMP |
| 4011 | KMKSKKCAG |
| 4012 | RKKKKQLGI |
| 4013 | KFRKRKIYS |
| 4014 | KIRKRRQHT |
| 4015 | KRKKKMRPA |
| 4016 | KAKKQKTTE |
| 4017 | SMKKFKKMS |
| 4018 | KKKVLKTVE |
| 4019 | KSKVKKHGW |
| 4020 | KQKKIIKCF |
| 4021 | KRKKNKMGP |
| 4022 | KSKKNKWQQ |
| 4023 | KKKRIQCHV |
| 4024 | KIKTKKKMA |
| 4025 | KRKRCVWRS |
| 4026 | KKRKKNTFS |
| 4027 | PKKKKRWTP |
| 4028 | VKKSKKQTN |
| 4029 | KKKAKHIEC |
| 4030 | KAKKNKGHY |
| 4031 | VKHRKKVKY |
| 4032 | KTKQKKHCC |
| 4033 | KTHKKKGSY |
| 4034 | IKKSKKAFG |
| 4035 | TRKIKKQKG |
| 4036 | KAKKMKMSW |
| 4037 | KKKRSLKIE |
| 4038 | KKKRKKTIY |
| 4039 | KAKHKKQIE |
| 4040 | CRKKKRRMW |
| 4041 | KKRRKGMGF |
| 4042 | KVRKAKVQQ |
| 4043 | CKKKKSVCF |
| 4044 | MAKRKKRRH |
| 4045 | MKHKKKNHV |
| 4046 | KSKKRKMTT |
| 4047 | KCKKNKHHP |
| 4048 | KKKRLRGRG |
| 4049 | IVKKGKHKS |
| 4050 | SKRKKKIVW |
| 4051 | KTKMRKKNA |
| 4052 | KKKYSKNTC |
| 4053 | AKKKKGMKW |
| 4054 | KSKKGKTSP |
| 4055 | MIKKIKKCG |
| 4056 | RFKRKKMKM |
| 4057 | HTRKMKTHF |
| 4058 | KTKKSKVAM |
| 4059 | GRKKRKKSD |
| 4060 | KRKCIKQKF |
| 4061 | KRKKGRKAE |
| 4062 | QQKKKKATW |
| 4063 | KRRKRGYST |
| 4064 | KMKRYRWHY |
| 4065 | CMKRKKKQP |
| 4066 | RKKKKWWST |
| 4067 | KKKKYQVFT |
| 4068 | KIKMYKKFN |
| 4069 | SKKKQKCLV |
| 4070 | RKRKKRRIY |
| 4071 | IKKRYKHLV |
| 4072 | KIKYSKKTG |
| 4073 | NAKRYRQHY |
| 4074 | KRRRIQRSM |
| 4075 | KVKKKGRSD |
| 4076 | RTKRNRKCQ |
| 4077 | KKRSKSKCS |
| 4078 | KCKNAKKVG |
| 4079 | HKRRCRHRY |
| 4080 | RHKKKKEHC |
| 4081 | HKKTKKNSS |
| 4082 | KCKKRKFMP |
| 4083 | KNKRVKKMG |
| 4084 | MKRRKKKWI |
| 4085 | NKKNKKVNN |
| 4086 | AVKRKRRMQ |
| 4087 | KKKRQRGWF |
| 4088 | KSKTKSKVC |
| 4089 | KVKHVKKQS |
| 4090 | NNKKRKKFQ |
| 4091 | IKKMKKARM |
| 4092 | KVKLKKICH |
| 4093 | KFHKKKQSW |
| 4094 | KCKKWKIMM |
| 4095 | KGKSGKTKM |
| 4096 | VRRRKKRWA |
| 4097 | KKKAKNYTY |
| 4098 | HKKRKPTVY |
| 4099 | KVRKRRYCA |
| 4100 | KKKRRRNAW |
| 4101 | KSKSKKMFR |
| 4102 | KKKRKVYGM |
| 4103 | SRRRKKNKH |
| 4104 | RKRKKRYHM |
| 4105 | KPKRKSCKH |
| 4106 | KTKRRKSMC |
| 4107 | AKKKFKYNN |
| 4108 | RRKRIKSSQ |
| 4109 | KKKGRRKNR |
| 4110 | KAKGFKKMD |
| 4111 | RRKKAKHVH |
| 4112 | YKKRKKCGM |
| 4113 | KARRMKRQK |
| 4114 | SAKKKKWDS |
| 4115 | VHKKKKSCW |
| 4116 | QVKVKKKCL |
| 4117 | KAKRFRWRS |
| 4118 | KKKIYKLHN |
| 4119 | KKHMKKSQV |
| 4120 | RRRRKKFTP |
| 4121 | KKKWIKGWG |
| 4122 | QAKKKKVHA |
| 4123 | QKKKMRKFS |
| 4124 | KKRCKKCSS |
| 4125 | KRHRAKKKH |
| 4126 | MAKRYRWWS |
| 4127 | KKKYFKKVY |
| 4128 | KKKKKPRSG |
| 4129 | RQKKRKAGS |
| 4130 | KRKRRIGRT |
| 4131 | KSKKKIRAS |
| 4132 | KRRKMRQVS |
| 4133 | LVKKSKKNG |
| 4134 | RKKKKMGWN |
| 4135 | KAKVHKKRN |
| 4136 | KKRLCKWKV |
| 4137 | KSRKMKFMC |
| 4138 | GGRRKKRYY |
| 4139 | LKKKTGRKP |
| 4140 | KAKRCKRWY |
| 4141 | LYKKRKKCI |
| 4142 | KAKAFKSSQ |
| 4143 | SKRKNKSCT |
| 4144 | KOKKKGDAR |
| 4145 | ILKRKKKSL |
| 4146 | KVKKKQVNG |
| 4147 | HRRRTKRFQ |
| 4148 | KVKKKTVGP |
| 4149 | KALKKKGWG |
| 4150 | SKKRKKQKH |
| 4151 | KVKPKKHLA |
| 4152 | KKKSIKVVP |
| 4153 | KKRRAKNCP |
| 4154 | KTRKGKKHG |
| 4155 | ISRKKKQAG |
| 4156 | KAKKKRNQA |
| 4157 | VKKRKRVWT |
| 4158 | QKKRQRYPS |
| 4159 | KKKGVKNRS |
| 4160 | KRRKIKKGG |
| 4161 | KRKRPRQSC |
| 4162 | RKRKRIGGT |
| 4163 | KKKRSRRSC |
| 4164 | RKKKRCKSS |
| 4165 | KTKKPKQHP |
| 4166 | HKKKKCNWC |
| 4167 | KQRKIKRYP |
| 4168 | KTKKKQRAN |
| 4169 | KSKNKTTKC |
| 4170 | RFKRKKFIS |
| 4171 | KKKMKSRHP |
| 4172 | KCKSKRHHK |
| 4173 | KRKTKKHPQ |
| 4174 | KSRKTKYCK |
| 4175 | RTKRSRSNY |
| 4176 | KRARKKAKC |
| 4177 | AAKRKKKCC |
| 4178 | KKKRYRRYW |
| 4179 | TKKRYKRMY |
| 4180 | KKRRIRNYG |
| 4181 | KRKKNLKAN |
| 4182 | VLKHKKKSC |
| 4183 | KTCKKKWMG |
| 4184 | KRRKLRRGG |
| 4185 | KNKKKHWRP |
| 4186 | KSRKTKTGW |
| 4187 | KKSRKRKQF |
| 4188 | HKKGFKKWY |
| 4189 | KAKRKKWLH |
| 4190 | KKKRFVMRG |
| 4191 | KTKKQRKAV |
| 4192 | KGKKSKQGG |
| 4193 | RKKQQKNKA |
| 4194 | KFRKKHTLP |
| 4195 | RKKRYRWCQ |
| 4196 | HKKKKHFYA |
| 4197 | VKKRNRRTM |
| 4198 | RKKKMKGAG |
| 4199 | KKKSMARKW |
| 4200 | KTKKTKAWG |
| 4201 | KTRRKKHTD |
| 4202 | LKRKKPKCQ |
| 4203 | QVKGKKAKP |
| 4204 | IKKRRRFGP |
| 4205 | NGKHKKKCF |
| 4206 | KMKACKKAG |
| 4207 | KKKRNKIYS |
| 4208 | KKKKGNYLT |
| 4209 | KKKQYKSWV |
| 4210 | KFKKMKRKM |
| 4211 | KQKRSKAYC |
| 4212 | KGKKRGRQM |
| 4213 | KKKKHWFVA |
| 4214 | KARWKKRHG |
| 4215 | RKKRKRRNN |
| 4216 | KKCKKKVPA |
| 4217 | VTKKKKVRT |
| 4218 | SVRKKKKAA |
| 4219 | AKKHMKKVG |
| 4220 | TKKKKRYHN |
| 4221 | KWRKFKCYH |
| 4222 | HKRKRHMNP |
| 4223 | RKKTKKAFT |
| 4224 | IQKKKKCWL |
| 4225 | KYRKYKTSS |
| 4226 | KSKRYKTCC |
| 4227 | VKKKSKVTS |
| 4228 | KSKRKGHNK |
| 4229 | RKKMKRQKE |
| 4230 | KNKRKAKWP |
| 4231 | KRKKRGSWG |
| 4232 | KARRVRRYY |
| 4233 | QKKKVKKCW |
| 4234 | VRKRQRQCT |
| 4235 | RKRKWKCHC |
| 4236 | KVKKKAVVY |
| 4237 | KVKKVKISP |
| 4238 | KKKKHQPVA |
| 4239 | KRRRKTGSQ |
| 4240 | QNKKKKAYI |
| 4241 | AKKKCKKWT |
| 4242 | TKKRRKSPS |
| 4243 | KCKRRKKAG |
| 4244 | KNKRRKKLY |
| 4245 | KVKASKRKC |
| 4246 | KKKRFSQVF |
| 4247 | KGKWKKFIG |
| 4248 | HIRKKKYGN |
| 4249 | KLKWKKMWC |
| 4250 | KRRKNRYWA |
| 4251 | HKKKIKRCD |
| 4252 | KCKKKSTYS |
| 4253 | KKFKVKHIC |
| 4254 | QKKVMKKCG |
| 4255 | HKRKICGHH |
| 4256 | KVKKKRCRK |
| 4257 | KTRKKNHGT |
| 4258 | KKQKKRSQN |
| 4259 | KKARRKRYH |
| 4260 | KSKKHKHDH |
| 4261 | KSKDKKQWQ |
| 4262 | AKKRKRVAW |
| 4263 | SPKKKKWRT |
| 4264 | KFKKTKWKF |
| 4265 | KRKVKKLMF |
| 4266 | KFRKVKTAT |
| 4267 | YKKMKKCAY |
| 4268 | RKKKDRRKC |
| 4269 | HKRRRKTPP |
| 4270 | KAKKKNFVI |
| 4271 | GKKHTKGKQ |
| 4272 | KMKSKKMQQ |
| 4273 | RAKRYKKTA |
| 4274 | NCKRKKGNK |
| 4275 | KKKHMKIGN |
| 4276 | KYKFKKWST |
| 4277 | KSKRYRMMV |
| 4278 | KKKKAKMDG |
| 4279 | TNKKKKKAV |
| 4280 | KLKKFKRDW |
| 4281 | KQKRRRRCM |
| 4282 | KKKWLKNIY |
| 4283 | KVKMKKIWA |
| 4284 | KKKSSKSFC |
| 4285 | KKRKHINKA |
| 4286 | KRKRKRRCH |
| 4287 | YKKKMKWML |
| 4288 | AKKGKKQMT |
| 4289 | CKKFQKKFA |
| 4290 | NKRRIKHRQ |
| 4291 | IKKRRKTQN |
| 4292 | RKKQKKRTS |
| 4293 | RSKRRKACG |
| 4294 | YGRKKKQCK |
| 4295 | RTKKKKVCS |
| 4296 | KAKTKGYKN |
| 4297 | KSKKKRNQQ |
| 4298 | KLKRTRRSW |
| 4299 | RTRKVKHKC |
| 4300 | KVKRYRNHW |
| 4301 | QRRRNKRQQ |
| 4302 | KKRRYHSWA |
| 4303 | KKKSKKNSA |
| 4304 | KVKWKKDQG |
| 4305 | VKKGMKKQY |
| 4306 | KVRRKRRPS |
| 4307 | YVRKKKFGG |
| 4308 | KVKKKRIQF |
| 4309 | MKGKKKCAG |
| 4310 | KKKWVKTFW |
| 4311 | NKKHRKKGH |
| 4312 | KKKRHQVVK |
| 4313 | MWKRKKVKC |
| 4314 | KGRRKRSAM |
| 4315 | KCRKKQAKN |
| 4316 | KKRKHARVH |
| 4317 | KLKVKKVGS |
| 4318 | KCKRQKRCA |
| 4319 | HIKFKKKQC |
| 4320 | AKKNKKWFT |
| 4321 | KQKIVKKWM |
| 4322 | KTKRIRHHQ |
| 4323 | KIRKKRRRL |
| 4324 | GFKKKKKQQ |
| 4325 | RKKRGKCYG |
| 4326 | KVKKRKTNG |
| 4327 | KKKRRSNLS |
| 4328 | KRKSGKNKS |
| 4329 | KKRKTHATK |
| 4330 | KCKKRGRTK |
| 4331 | QKKCKKRAI |
| 4332 | QKKFRKKQQ |
| 4333 | KQKRIKKLG |
| 4334 | KKKLKGWHV |
| 4335 | KKKKMYFSY |
| 4336 | KTKKVKYWN |
| 4337 | RKKFLKRKG |
| 4338 | IKKRKKTQW |
| 4339 | KFKPKKAYA |
| 4340 | KKRKRSAKS |
| 4341 | LAKPKKKAN |
| 4342 | KVKRKSNKT |
| 4343 | KKKCKLAMF |
| 4344 | KKKANKHMD |
| 4345 | KTRKRRLYA |
| 4346 | RKRRHKGRP |
| 4347 | RKKKKQASY |
| 4348 | KKKKTRHKS |
| 4349 | HKKIKKYVS |
| 4350 | KKKLGKCRW |
| 4351 | QKRKIRRMA |
| 4352 | RHKKKRHCQ |
| 4353 | KKKKGKGIA |
| 4354 | KKKKYWMIH |
| 4355 | KKKRPKATT |
| 4356 | KGKKKSVYP |
| 4357 | KSKKKRRFD |
| 4358 | MIKKVKTKG |
| 4359 | KSKKKAQMH |
| 4360 | CKKYKKYNY |
| 4361 | KKRRTRHSF |
| 4362 | KHKAMKFKN |
| 4363 | HKRKKKLGV |
| 4364 | KVRRKKNAP |
| 4365 | KKKITKRGA |
| 4366 | MKKKKKVCC |
| 4367 | KVRKRKKCA |
| 4368 | KFKRKNCKQ |
| 4369 | SVKKKKNVS |
| 4370 | GPKKKKWSS |
| 4371 | RKKRHGKGC |
| 4372 | KVKMCKQKW |
| 4373 | HKRKGHWVQ |
| 4374 | KFKKKINFW |
| 4375 | RKKRGRKES |
| 4376 | TKKRKPKGS |
| 4377 | HKKRNRFCT |
| 4378 | KRKCKAKGN |
| 4379 | KTRKSKKDR |
| 4380 | KIKRKRKHM |
| 4381 | TKRRHKRQV |
| 4382 | KKKTCKLGH |
| 4383 | KKKKAQRHT |
| 4384 | KKKCKRKYA |
| 4385 | HKKNIKKHW |
| 4386 | EKKRKKWYS |
| 4387 | KKKHCKLHW |
| 4388 | KLKSKKRGR |
| 4389 | STKRRRRAS |
| 4390 | KRKKSMMKT |
| 4391 | AKKKIKKVG |
| 4392 | KRKRWKKLF |
| 4393 | KVKKSKAHM |
| 4394 | KKRRWRHHH |
| 4395 | RSRKVKRHY |
| 4396 | KKKKTLHTN |
| 4397 | CKKKRKQHC |
| 4398 | TKKTKKQQP |
| 4399 | KKKRLKHSD |
| 4400 | MARKKKSRT |
| 4401 | NKKPRKVKW |
| 4402 | VKKRKRNHV |
| 4403 | KVKKMKWKR |
| 4404 | KKRKNKEFI |
| 4405 | KKKGKRHRG |
| 4406 | LAKHKKHKH |
| 4407 | KQKKMKRQD |
| 4408 | KAKWKKWSW |
| 4409 | LKRKKRKDC |
| 4410 | KKRKWHYYG |
| 4411 | KAKKKHSRV |
| 4412 | KQKKKRRTT |
| 4413 | KKKCKNSGT |
| 4414 | QKRKRKLSE |
| 4415 | CKKLKKTGY |
| 4416 | RKKKPKMQM |
| 4417 | RRRRSKSKA |
| 4418 | HKKSWKKYP |
| 4419 | KKKWKVVNW |
| 4420 | KKKVQKMHP |
| 4421 | KKRKKGSWA |
| 4422 | KIRRYKRQA |
| 4423 | KKFKKKSAN |
| 4424 | SKRKKRKWG |
| 4425 | MSRKKKRST |
| 4426 | KKRRHGRWY |
| 4427 | KKKKSSTGP |
| 4428 | KVRKTKIGE |
| 4429 | KKRRTCRHQ |
| 4430 | KKRKGIRGT |
| 4431 | HKRKKHNQM |
| 4432 | RKKRHKGNQ |
| 4433 | LCKKKKRTC |
| 4434 | LKKPKKTCN |
| 4435 | KIKHKKWLV |
| 4436 | KKKKKIQMQ |
| 4437 | KVKRKQQLC |
| 4438 | RCRKRKSIC |
| 4439 | SKKRRRRMS |
| 4440 | ICKKKRLKG |
| 4441 | KLRKFKGKY |
| 4442 | KLKRKKNVQ |
| 4443 | RMKKKRQIM |
| 4444 | KKRRQKVKH |
| 4445 | KKKSKKMFE |
| 4446 | HRKRVRVRF |
| 4447 | KMKKKRCPA |
| 4448 | SARKKKWCC |
| 4449 | HKKRKQTGT |
| 4450 | KKKACKKSA |
| 4451 | KIKKTRKNH |
| 4452 | VKKKLKTGA |
| 4453 | KFKARKHKT |
| 4454 | KHKSHKKQL |
| 4455 | RKNKKKHNA |
| 4456 | CRKKKKIKC |
| 4457 | HHKRQRAHH |
| 4458 | CVRKKKQVG |
| 4459 | SFKWKKKQY |
| 4460 | HGKQKKKCC |
| 4461 | KARRMKNWS |
| 4462 | KKKRFGRQK |
| 4463 | KNKKMKKAA |
| 4464 | KTKVKKVAH |
| 4465 | KKKNYKSSY |
| 4466 | QKKGRKKNL |
| 4467 | TRRKKRRPC |
| 4468 | KGKVSKKQS |
| 4469 | RNRKKKWCC |
| 4470 | KKKKRKNHR |
| 4471 | LHKKKKGNW |
| 4472 | HTKKKKFAP |
| 4473 | NKKKFKTVQ |
| 4474 | KFRKKRWVP |
| 4475 | KKKKQNHGC |
| 4476 | VNKKKKHAA |
| 4477 | SMKKKRKAS |
| 4478 | SKKKQKARP |
| 4479 | TGKKKKHYW |
| 4480 | YCKKWKIKN |
| 4481 | TKRKKKQFH |
| 4482 | KKQKKHRMP |
| 4483 | KTRNKKPKC |
| 4484 | HAKGKKKCM |
| 4485 | KKKKCPHAQ |
| 4486 | KKKRVKDKQ |
| 4487 | KKKTQRHHN |
| 4488 | KKKWWKKSQ |
| 4489 | KVKRKGHCQ |
| 4490 | KVKRSKHCQ |
| 4491 | KIRKKKDMQ |
| 4492 | KKKIRKNFY |
| 4493 | SKRKYKKTG |
| 4494 | KKKPKHVCA |
| 4495 | KRRKHIRRH |
| 4496 | CRKKTKQKV |
| 4497 | RARKKKCFF |
| 4498 | IKKCKKRVM |
| 4499 | HKKRNRKSK |
| 4500 | KKRFKKAFG |
| 4501 | CIKKKKYMN |
| 4502 | KRKKQYKVC |
| 4503 | KMKTYKIKQ |
| 4504 | KHQKKKRSG |
| 4505 | YKKMKKAMI |
| 4506 | KAKKKKKKF |
| 4507 | HVKKKKWNG |
| 4508 | KKRCKKHGS |
| 4509 | YKKKTKYSS |
| 4510 | KKRRYRHAG |

In some embodiments, a retina tissue-tropic VP capsid polypeptide comprises a 581 to 589 region having at least 75% sequence identity to any one of SEQ ID NO: 3576-SEQ ID NO: 4510. In some embodiments, a retina tissue-tropic VP capsid polypeptide comprises a 581 to 589 region having at least 77.7% sequence identity to any one of SEQ ID NO: 3576-SEQ ID NO: 4510. In some embodiments, a retina tissue-tropic VP capsid polypeptide comprises a 581 to 589 region having at least 85% sequence identity to any one of SEQ ID NO: 3576-SEQ ID NO: 4510. In some embodiments, a retina tissue-tropic VP capsid polypeptide comprises a 581 to 589 region having at least 88.8% sequence identity to any one of SEQ ID NO: 3576-SEQ ID NO: 4510. In some embodiments, a retina tissue-tropic VP capsid polypeptide comprises a 581 to 589 region having a sequence of any one of SEQ ID NO: 3576-SEQ ID NO: 4510. In some embodiments, a retina tissue-tropic VP capsid polypeptide comprises a 581 to 589 region having one or two amino acid substitutions relative to any one of SEQ ID NO: 3576-SEQ ID NO: 4510. In some embodiments, a retina tissue-tropic VP capsid polypeptide comprises a 581 to 589 region having one or two conservative amino acid substitutions relative to any one of SEQ ID NO: 3576-SEQ ID NO: 4510.

TABLE 8 provides additional 581 to 589 region sequences predicted to confer retina tissue tropism or preference, generated and selected using machine learning.

TABLE 8
Additional Exemplary 581 to 589 Region
Sequences for Retinal Targeting

| SEQ ID NO | 581 to 589 Region |
|---|---|
| 4511 | KHKKKGGPQ |
| 4512 | RKQKKKAWC |
| 4513 | KKRKRAPKI |

In some embodiments, a retina tissue-tropic VP capsid polypeptide comprises a 581 to 589 region having at least 75% sequence identity to any one of SEQ ID NO: 4511-SEQ ID NO: 4513. In some embodiments, a retina tissue-tropic VP capsid polypeptide comprises a 581 to 589 region having at least 77.7% sequence identity to any one of SEQ ID NO: 4511-SEQ ID NO: 4513. In some embodiments, a retina tissue-tropic VP capsid polypeptide comprises a 581 to 589 region having at least 85% sequence identity to any one of SEQ ID NO: 4511-SEQ ID NO: 4513. In some embodiments, a retina tissue-tropic VP capsid polypeptide comprises a 581 to 589 region having at least 88.8% sequence identity to any one of SEQ ID NO: 4511-SEQ ID NO: 4513. In some embodiments, a retina tissue-tropic VP capsid polypeptide comprises a 581 to 589 region having a sequence of any one of SEQ ID NO: 4511-SEQ ID NO: 4513. In some embodiments, a retina tissue-tropic VP capsid polypeptide comprises a 581 to 589 region having one or two amino acid substitutions relative to any one of SEQ ID NO: 4511-SEQ ID NO: 4513. In some embodiments, a retina tissue-tropic VP capsid polypeptide comprises a 581 to 589 region having one or two conservative amino acid substitutions relative to any one of SEQ ID NO: 4511-SEQ ID NO: 4513.

Features for Retina Tissue-Tropic AAV VP Capsid Polypeptides. The present disclosure provides features of 581 to 589 region sequences that enhance retina tissue tropism or preference, as determined from a primary screen. In some embodiments, a 581 to 589 region with retina tissue tropism or preference contains one or more basic amino acid residues (e.g., K, R, or H). In some embodiments, a 581 to 589 region with retina tissue tropism or preference contains two or more basic amino acid residues (e.g., K, R, or H). In some embodiments, a 581 to 589 region with retina tissue tropism or preference contains three or more basic amino acid residues (e.g., K, R, or H). In some embodiments, a 581 to 589 region with retina tissue tropism or preference contains four or more basic amino acid residues (e.g., K, R, or H). In some embodiments, a 581 to 589 region with retina tissue tropism or preference contains one basic amino acid residue (e.g., K, R, or H). In some embodiments, a 581 to 589 region with retina tissue tropism or preference contains two basic amino acid residues (e.g., K, R, or H). In some embodiments, a 581 to 589 region with retina tissue tropism or preference contains three basic amino acid residues (e.g., K, R, or H). In some embodiments, a 581 to 589 region with retina tissue tropism or preference contains four basic amino acid residues (e.g., K, R, or H). In some embodiments, a 581 to 589 region with retina tissue tropism or preference contains five basic amino acid residues (e.g., K, R, or H). In some embodiments, a 581 to 589 region with retina tissue tropism or preference contains six basic amino acid residues (e.g., K, R, or H). In some embodiments, a 581 to 589 region with retina tissue tropism or preference contains seven basic amino acid residues (e.g., K, R, or H). In some embodiments, a 581 to 589 region with retina tissue tropism or preference contains eight basic amino acid residues (e.g., K, R, or H). In some embodiments, a 581 to 589 region with retina tissue tropism or preference contains nine basic amino acid residues (e.g., K, R, or H).

In some embodiments, a 581 to 589 region with retina tissue tropism or preference contains no more than three acidic amino acid residues (e.g., D or E). In some embodiments, a 581 to 589 region with retina tissue tropism or preference contains no more than two acidic amino acid residues (e.g., D or E). In some embodiments, a 581 to 589 region with retina tissue tropism or preference contains no more than one acidic amino acid residues (e.g., D or E). In some embodiments, a 581 to 589 region with retina tissue tropism or preference contains no acidic amino acid residues (e.g., D or E). In some embodiments, a 581 to 589 region with retina tissue tropism or preference contains one acidic amino acid residue (e.g., D or E). In some embodiments, a 581 to 589 region with retina tissue tropism or preference contains two acidic amino acid residues (e.g., D or E). In some embodiments, a 581 to 589 region with retina tissue tropism or preference contains three acidic amino acid residues (e.g., D or E).

In some embodiments, a 581 to 589 region with retina tissue tropism or preference contains more basic amino acid residues (e.g., K, R, or H) than acidic amino acid residues (e.g., D or E). In some embodiments, a 581 to 589 region with retina tissue tropism or preference contains one or more basic amino acid residues (e.g., K, R, or H) and no acidic amino acid residues (e.g., D or E). In some embodiments, a 581 to 589 region with retina tissue tropism or preference contains two or more basic amino acid residues (e.g., K, R, or H) and no more than one acidic amino acid residue (e.g., D or E). In some embodiments, a 581 to 589 region with retina tissue tropism or preference contains two or more basic amino acid residues (e.g., K, R, or H) and no acidic amino acid residues (e.g., D or E). In some embodiments, a 581 to 589 region with retina tissue tropism or preference contains three or more basic amino acid residues (e.g., K, R, or H) and no more than two acidic amino acid residues (e.g., D or E).

In some embodiments, a 581 to 589 region having a sequence of SEQ ID NO: 5014 (X¹X²X³X⁴X⁵X⁶X⁷X⁸X⁹, wherein X¹, X², X³, X⁴, X⁵, X⁶, X⁷, X⁸, and X⁹ are each independently any amino acid) comprises K, R, or H at position X³. In some embodiments, a 581 to 589 region having a sequence of SEQ ID NO: 5014 comprises K or R at position X³. In some embodiments, a 581 to 589 region having a sequence of SEQ ID NO: 5014 comprises K at position X³. In some embodiments, a 581 to 589 region having a sequence of SEQ ID NO: 5014 comprises R at position X³. In some embodiments, a 581 to 589 region having a sequence of SEQ ID NO: 5014 comprises K, R, or H at position X⁶. In some embodiments, a 581 to 589 region having a sequence of SEQ ID NO: 5014 comprises K or R at position X⁶. In some embodiments, a 581 to 589 region having a sequence of SEQ ID NO: 5014 comprises K at position X⁶. In some embodiments, a 581 to 589 region having a sequence of SEQ ID NO: 5014 comprises R at position X⁶. In some embodiments, a 581 to 589 region having a sequence of SEQ ID NO: 5014 comprises K, R, or H at position X¹. In some embodiments, a 581 to 589 region having a sequence of SEQ ID NO: 5014 comprises K or R at position X¹. In some embodiments, a 581 to 589 region having a sequence of SEQ ID NO: 5014 comprises K at position X¹. In some embodiments, a 581 to 589 region having a sequence of SEQ ID NO: 5014 comprises R at position X¹. In some embodiments, a 581 to 589 region having a sequence of SEQ ID NO: 5014 comprises K, R, or H at position X⁴. In some embodiments, a 581 to 589 region having a sequence of SEQ ID NO: 5014 comprises K or R at position X⁴. In some embodiments, a 581 to 589 region having a sequence of SEQ ID NO: 5014 comprises K at position X⁴. In some embodiments, a 581 to 589 region having a sequence of SEQ ID NO: 5014 comprises R at position X⁴.

A VP capsid polypeptide may have a 581 to 589 region having a sequence of SEQ ID NO: 5014, wherein X¹ of SEQ ID NO: 5014 is K or R. In some embodiments, the 581 to 589 region has a sequence of any one of SEQ ID NOs: 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2343, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2185, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, or 1555.

A VP capsid polypeptide may have a 581 to 589 region having a sequence of SEQ ID NO: 5014, wherein X² of SEQ ID NO: 5014 is K or R. In some embodiments, the 581 to 589 region has a sequence of any one of SEQ ID NOs: 752, 2144, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 1308, 1230, 3960, 1378, 2994, 3080, 2589, 3681, 51, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 778, 87, 3891, 4456, 4040, 4496, 1982, 2001, 1478, 1189, 120, 4791, 2158, 121, 803, 804, 1463, 805, 122, 2139, 2290, 1760, 2465, 123, 130, 131, 132, 4933, 808, 2336, 2026, 2204, 809, 810, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 234, 4962, 2269, 843, 235, 844, 236, 237, 4930, 845, 846, 238, 847, 1295, 3601, 3877, 1930, 1420, 1678, 1460, 285, 1626, 299, 3809, 4271, 3844, 1436, 300, 1515, 1617, 3646, 4059, 1431, 1331, 310, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 1959, 1840, 3736, 1283, 3861, 1383, 3644, 3691, 4259, 1749, 1418, 1176, 4216, 1924, 1421, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 954, 955, 2437, 4139, 1482, 3602, 4434, 3721, 1592, 1825, 1698, 1519, 1352, 4202, 4409, 1977, 1917, 2382, 3675, 1833, 3974, 419, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 3879, 2698, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 1019, 2012, 1803, 3642, 497, 1322, 1261, 4027, 3478, 3763, 2959, 1293, 2462, 517, 3197, 1595, 3413, 1675, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1050, 3515, 2219, 1433, 1323, 4301, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2324, 605, 1368, 3827, 4103, 606, 607, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 4035, 2910, 3391, 2808, 3099, 2913, 3676, 2852, 3284, 2731, 1609, 2375, 2201, 3537, 3994, 1319, 4467, 1802, 1227, 1623, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 4879, 1841, 4234, 4924, 1154, 1314, 4096, 2023, 3815, 2005, 1964, 3439, 2833, 4287, 4509, 4505, 4267, 3697, 3272, 4112, 2967, 3651, 1991, 1805, 1628, 3705, or 2250.

A VP capsid polypeptide may have a 581 to 589 region having a sequence of SEQ ID NO: 5014, wherein X³ of SEQ ID NO: 5014 is K or R. In some embodiments, the 581 to 589 region has a sequence of any one of SEQ ID NOs: 4177, 1289, 740, 741, 18, 745, 3477, 1548, 3051, 2982, 2877, 3036, 3426, 3073, 3169, 747, 3362, 2784, 21, 3192, 23, 1638, 748, 1703, 1836, 3660, 29, 750, 1640, 33, 34, 35, 36, 37, 38, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 3327, 3178, 3006, 3252, 2991, 3470, 2888, 2572, 3418, 2642, 2519, 3363, 40, 2633, 41, 42, 43, 755, 45, 756, 46, 48, 1952, 758, 49, 3122, 2551, 3718, 3484, 3577, 759, 1230, 3960, 1378, 2994, 3080, 2589, 3681, 760, 53, 3925, 55, 3654, 56, 61, 4086, 1740, 1660, 765, 766, 62, 1966, 63, 64, 768, 771, 65, 66, 67, 68, 3816, 2599, 2706, 74, 3865, 1549, 1361, 3882, 3713, 1942, 1312, 4501, 76, 77, 1560, 4289, 3757, 79, 3593, 4043, 1806, 3908, 4397, 1615, 4415, 3723, 2006, 3754, 1470, 4360, 3945, 2854, 3523, 3669, 82, 4065, 83, 1294, 1484, 3891, 4456, 4040, 4496, 1982, 2001, 1478, 1189, 1490, 1885, 1926, 89, 90, 91, 779, 1180, 92, 785, 786, 94, 95, 1831, 1867, 4458, 97, 1970, 1936, 1590, 100, 789, 790, 1298, 101, 1688, 791, 792, 2007, 103, 794, 795, 104, 105, 106, 1849, 108, 1579, 110, 1906, 113, 1444, 1344, 116, 802, 117, 118, 121, 803, 804, 1463, 805, 122, 124, 125, 127, 807, 132, 809, 810, 133, 1978, 812, 135, 136, 137, 138, 813, 140, 815, 1326, 141, 142, 143, 144, 145, 1634, 1302, 817, 146, 818, 147, 148, 149, 151, 152, 153, 154, 155, 819, 820, 157, 159, 160, 163, 164, 165, 166, 822, 167, 168, 169, 170, 171, 823, 824, 1651, 173, 1483, 174, 175, 825, 176, 177, 826, 827, 181, 828, 182, 829, 184, 185, 186, 187, 188, 189, 830, 191, 192, 193, 194, 195, 196, 1391, 197, 200, 201, 202, 203, 204, 205, 832, 833, 206, 834, 836, 1334, 837, 3901, 3312, 2649, 1301, 211, 212, 1450, 4386, 3028, 213, 1446, 214, 215, 1896, 1522, 3490, 838, 217, 218, 219, 1612, 220, 221, 222, 839, 223, 840, 224, 226, 227, 230, 231, 842, 232, 233, 844, 236, 237, 851, 852, 853, 1536, 240, 854, 242, 243, 244, 245, 246, 247, 249, 250, 251, 252, 253, 254, 255, 857, 256, 257, 258, 259, 858, 859, 260, 261, 860, 861, 862, 262, 264, 1241, 265, 266, 866, 1477, 267, 867, 869, 870, 271, 871, 872, 272, 1252, 273, 1591, 275, 1980, 276, 876, 877, 277, 278, 279, 1892, 1539, 880, 1295, 3601, 3877, 1930, 1420, 1678, 1460, 282, 1799, 283, 1626, 1374, 287, 1620, 1290, 1354, 888, 291, 1956, 889, 292, 4324, 293, 4138, 891, 3903, 297, 3829, 3809, 4271, 3844, 1436, 1515, 1617, 1961, 304, 306, 1589, 4370, 308, 309, 894, 3646, 4059, 1431, 1331, 312, 895, 1779, 313, 314, 315, 316, 896, 1558, 1552, 898, 321, 322, 899, 1958, 901, 325, 902, 326, 1491, 329, 330, 905, 4484, 1756, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 1888, 4248, 1557, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 3435, 3482, 3020, 2748, 2646, 2787, 2760, 2563, 3085, 3133, 3158, 3170, 2703, 3494, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 2979, 2542, 1762, 4472, 1472, 1529, 4057, 1976, 4507, 1583, 338, 911, 1711, 340, 912, 2013, 341, 4440, 917, 344, 918, 919, 921, 922, 347, 351, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 4145, 354, 925, 1782, 926, 927, 3610, 357, 359, 4224, 1918, 1959, 3736, 1770, 931, 4155, 364, 1279, 365, 1543, 367, 4049, 1633, 1722, 368, 369, 1495, 1566, 370, 1211, 1873, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 3741, 1938, 3709, 1727, 3893, 1280, 3622, 1784, 3834, 1752, 1983, 1743, 3979, 4461, 4113, 3817, 4232, 1340, 1489, 4214, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 377, 1395, 4453, 3940, 3778, 4374, 4210, 4009, 4264, 4339, 4368, 1738, 378, 1610, 4194, 4474, 4013, 4266, 1771, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 2002, 1657, 1663, 1336, 3894, 1554, 1494, 1405, 3576, 4314, 3695, 1851, 4362, 1815, 3845, 4511, 3656, 3638, 1406, 1596, 4454, 1443, 1661, 1359, 1734, 3650, 1910, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1680, 4441, 3628, 3725, 1945, 4206, 1588, 1439, 3794, 4447, 1838, 3776, 3658, 4064, 1377, 4011, 4272, 4503, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 3613, 3273, 3452, 3525, 1229, 4105, 3835, 383, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 1520, 1767, 1466, 1194, 4137, 1908, 1927, 1758, 3772, 3661, 4186, 4174, 1170, 3850, 1315, 1611, 1530, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 1716, 3961, 1506, 4221, 1376, 1683, 4276, 1445, 1721, 1632, 3607, 1328, 1455, 1913, 1497, 1317, 1951, 1778, 1379, 3768, 4225, 1668, 390, 391, 4406, 4341, 1480, 395, 1714, 1422, 396, 397, 3618, 4433, 1177, 3898, 398, 949, 950, 399, 951, 400, 1965, 4471, 952, 402, 2475, 403, 404, 4139, 1482, 3602, 4434, 3721, 1592, 1519, 1352, 4202, 4409, 1977, 1917, 407, 408, 1631, 1684, 409, 3889, 958, 959, 412, 960, 414, 415, 961, 1905, 416, 963, 417, 3675, 1833, 3974, 420, 421, 965, 966, 969, 970, 422, 1897, 423, 4133, 3830, 972, 424, 973, 427, 1708, 4141, 3749, 431, 1619, 975, 4044, 3590, 1944, 4126, 1923, 1706, 4400, 434, 1606, 979, 2604, 2895, 436, 3448, 1692, 983, 3862, 4055, 4358, 984, 985, 1664, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 3535, 1306, 4084, 3282, 1780, 987, 988, 990, 451, 452, 991, 992, 1728, 994, 995, 454, 1696, 3116, 1869, 456, 1386, 999, 3879, 2698, 1001, 461, 1447, 462, 464, 4425, 1645, 1231, 1468, 472, 473, 476, 1273, 1007, 1744, 1488, 1659, 477, 1604, 481, 4313, 482, 483, 1499, 484, 1016, 4073, 1777, 1458, 4274, 487, 1775, 4205, 1526, 1697, 1017, 2000, 489, 3917, 3616, 4311, 1800, 4473, 4085, 4401, 1310, 3783, 3765, 491, 1948, 3699, 1735, 3792, 4290, 495, 4090, 496, 2012, 1803, 3642, 1322, 498, 1021, 1022, 502, 1333, 503, 1023, 1890, 510, 1026, 3608, 1732, 511, 2397, 1864, 1816, 1531, 3636, 1699, 1027, 1029, 1366, 2112, 1261, 4027, 3478, 3763, 2959, 517, 3197, 1595, 3413, 1448, 518, 1675, 1797, 520, 1999, 3759, 1215, 4122, 1730, 524, 526, 3966, 3911, 529, 3289, 1821, 530, 531, 1874, 1038, 536, 537, 3767, 1929, 1041, 1042, 3683, 542, 1338, 4331, 4332, 4466, 2719, 3803, 4123, 4233, 1824, 3129, 3087, 3505, 4158, 4254, 1449, 3720, 4351, 4414, 1673, 3453, 3254, 1649, 546, 4240, 3580, 1046, 1047, 550, 2598, 551, 552, 3819, 1259, 4062, 2841, 1050, 3515, 4301, 1052, 1053, 3738, 1567, 1270, 3857, 1055, 1257, 1238, 3662, 561, 562, 1058, 4203, 1899, 3981, 1062, 1303, 4116, 567, 1946, 3620, 1514, 4002, 3679, 3964, 3707, 3666, 1348, 4273, 1804, 4497, 1401, 4438, 1895, 1264, 572, 1912, 3939, 4170, 4056, 1271, 1065, 1521, 1221, 1451, 1654, 3934, 4080, 4352, 1700, 3739, 1565, 1419, 1811, 3950, 1690, 1397, 3716, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 1723, 1070, 3750, 3633, 4070, 4104, 4162, 4235, 1863, 4346, 1881, 1814, 1572, 4443, 3761, 1346, 1587, 1072, 3592, 576, 1891, 4469, 1275, 1950, 1963, 4129, 1296, 1551, 3733, 4111, 3993, 4108, 3949, 1287, 1713, 1736, 1995, 1435, 1424, 1830, 1705, 1861, 1197, 1254, 4120, 1545, 4417, 1469, 1785, 1236, 4293, 3839, 3983, 4395, 1957, 3928, 4295, 3715, 3855, 3609, 1990, 4076, 4175, 3927, 1757, 1650, 4299, 1773, 2004, 1267, 1364, 1462, 1879, 4007, 3729, 3665, 3722, 1691, 1624, 1751, 1394, 3924, 3952, 580, 581, 2003, 1343, 1559, 1250, 1555, 4114, 1818, 1075, 1076, 1077, 1704, 4448, 1078, 1079, 1786, 585, 3354, 1832, 3432, 2891, 2505, 1903, 1081, 1082, 4459, 1083, 1563, 590, 591, 1085, 594, 1453, 2559, 2590, 2738, 4478, 4069, 1809, 1327, 4150, 3260, 3481, 3672, 3625, 2008, 4439, 3852, 3997, 1411, 599, 3847, 1268, 4050, 3377, 4424, 1496, 4143, 4493, 3492, 1962, 600, 601, 4017, 4477, 1794, 4263, 3072, 2009, 1754, 1087, 1088, 3888, 1368, 3827, 4103, 606, 1090, 1571, 1091, 1438, 1092, 1093, 612, 1094, 3701, 4389, 613, 1603, 1332, 1715, 4369, 1652, 1096, 617, 1541, 618, 1921, 1733, 1373, 1870, 619, 4218, 1798, 1100, 1702, 1101, 1667, 1384, 623, 3700, 1788, 1600, 624, 626, 2771, 627, 1339, 3875, 628, 629, 3070, 4860, 2557, 2795, 2906, 3118, 1614, 1947, 4479, 3766, 632, 1104, 1105, 633, 1813, 636, 3706, 1110, 640, 1111, 641, 642, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 3095, 1286, 3624, 4481, 1432, 3511, 2735, 4381, 3295, 1585, 1114, 1766, 3221, 2643, 2739, 3920, 644, 1116, 645, 646, 647, 1801, 4279, 3762, 1637, 1509, 1119, 2965, 2369, 2695, 3293, 650, 651, 1120, 652, 1121, 3264, 655, 658, 659, 4035, 2910, 3391, 2808, 3099, 2913, 3676, 2852, 3284, 2731, 1609, 3994, 1319, 4467, 1802, 1227, 1623, 662, 663, 664, 1124, 1125, 1128, 1129, 668, 669, 1130, 670, 1131, 1527, 1132, 671, 1772, 1135, 672, 1933, 1731, 1793, 675, 676, 3342, 1138, 1139, 677, 679, 1142, 1143, 681, 1145, 682, 685, 3822, 689, 690, 691, 1147, 1601, 3905, 694, 695, 1414, 1150, 1795, 1372, 1695, 1866, 4115, 1719, 698, 1920, 699, 700, 1544, 4305, 1636, 1393, 2010, 3810, 4452, 3677, 4227, 1277, 1400, 4402, 4157, 4197, 1337, 1253, 1532, 4028, 3989, 1493, 1854, 1902, 1510, 4182, 706, 707, 708, 4476, 1245, 714, 1841, 4234, 1314, 4096, 717, 1155, 4217, 1156, 723, 1157, 1627, 725, 1159, 1953, 1160, 1201, 727, 3787, 3747, 730, 1846, 3815, 2005, 1964, 1822, 1262, 4480, 1537, 1291, 1318, 4294, 1582, 734, 3439, 2833, 4287, 4509, 4505, 4267, 3697, 3272, 4112, 2967, 3651, 1628, 3705, 1195, 3859, 1164, 1165, 1166, 1745, 1973, 1168, 3807, 4307, 1454, or 1781.

A VP capsid polypeptide may have a 581 to 589 region having a sequence of SEQ ID NO: 5014, wherein X⁴ of SEQ ID NO: 5014 is K or R. In some embodiments, the 581 to 589 region has a sequence of any one of SEQ ID NOs: 739, 4177, 1289, 15, 743, 3477, 3051, 2982, 2877, 746, 3036, 3426, 3073, 3169, 3362, 2784, 3192, 1638, 25, 1703, 1836, 3660, 30, 32, 752, 1653, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 1467, 3327, 3178, 3006, 3252, 2991, 3470, 2888, 2572, 3418, 2642, 2519, 3363, 2633, 757, 1952, 3122, 2551, 3718, 2216, 3484, 3577, 1308, 1230, 3960, 2994, 3080, 2589, 3681, 51, 761, 3925, 3654, 763, 764, 57, 4086, 1740, 767, 772, 2599, 2706, 70, 3865, 1549, 1361, 3882, 1942, 1312, 4501, 1413, 3593, 4043, 1806, 3908, 4397, 1615, 2006, 3945, 2854, 3523, 3669, 1356, 777, 4065, 84, 1484, 778, 87, 3891, 4456, 4040, 4496, 2001, 1478, 1189, 1490, 1885, 2270, 1180, 786, 4458, 1970, 1590, 98, 1298, 101, 1688, 794, 107, 1849, 797, 1579, 111, 112, 113, 1444, 801, 114, 115, 119, 120, 122, 1760, 123, 124, 806, 125, 126, 127, 128, 129, 130, 134, 811, 814, 139, 1326, 144, 816, 1302, 150, 152, 821, 160, 161, 162, 1651, 1483, 179, 180, 183, 185, 187, 1391, 198, 199, 831, 204, 833, 207, 3901, 3312, 2649, 1301, 4386, 3028, 1896, 1522, 3490, 216, 218, 1612, 225, 228, 229, 234, 239, 849, 850, 1536, 241, 246, 248, 855, 249, 252, 253, 261, 4966, 864, 263, 4749, 2296, 1241, 866, 268, 868, 269, 270, 873, 874, 1252, 273, 1591, 274, 1980, 878, 1539, 3601, 3877, 1930, 1420, 1678, 1460, 1799, 1374, 1290, 1354, 290, 2391, 4324, 294, 4138, 3903, 892, 3829, 298, 3844, 1436, 300, 1515, 1617, 1961, 303, 1589, 893, 4370, 3646, 4059, 1331, 1558, 1552, 320, 1958, 1491, 1635, 332, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3414, 3093, 2996, 3141, 3544, 3102, 2856, 2957, 3466, 3461, 2788, 2660, 3344, 3204, 2720, 3379, 2826, 2785, 3249, 3527, 3352, 2569, 4457, 2011, 3906, 1533, 1755, 1888, 4248, 1557, 3804, 1363, 1934, 1602, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 1852, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 1911, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 2662, 2875, 3223, 2878, 3285, 2757, 3057, 2951, 3519, 2579, 2853, 3387, 3401, 3493, 3393, 1608, 3365, 2885, 3574, 3081, 3007, 3568, 3152, 3367, 3358, 3468, 3290, 3550, 3132, 2726, 2879, 3190, 3309, 2613, 2765, 3454, 2851, 2881, 3232, 3458, 3326, 3353, 2927, 3212, 2950, 3376, 3208, 3062, 3612, 3195, 3385, 2686, 2602, 3569, 3123, 2727, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 2666, 3246, 3130, 3560, 3236, 2730, 3572, 1607, 3689, 2968, 1457, 1500, 1184, 3450, 2577, 2567, 2532, 3415, 2934, 2984, 2568, 4147, 3002, 2986, 910, 2979, 2542, 4472, 1472, 4057, 4507, 1711, 913, 916, 1594, 4440, 343, 918, 345, 348, 350, 1518, 3728, 3637, 3570, 3429, 2870, 2995, 4338, 3205, 2880, 3261, 1994, 3968, 4291, 4204, 4071, 352, 2820, 3912, 3287, 1218, 1190, 4145, 353, 355, 1782, 928, 3610, 358, 4224, 1918, 1959, 1840, 3736, 4155, 1543, 4049, 1633, 1495, 372, 1873, 1726, 1748, 4411, 4506, 4270, 4156, 3780, 4036, 4030, 4016, 1550, 3899, 1247, 1244, 4140, 3930, 4117, 4189, 4001, 3799, 3643, 3619, 4149, 1313, 3709, 1727, 3893, 1280, 3622, 1784, 3834, 1752, 3979, 4461, 4113, 3817, 4232, 3902, 1857, 1904, 1481, 1516, 3820, 3789, 4252, 4047, 4330, 4082, 4094, 3785, 3687, 4318, 4243, 3866, 1621, 4315, 1192, 1513, 1856, 3324, 2769, 2678, 3296, 375, 1395, 1584, 4093, 3940, 3778, 4374, 4210, 4009, 4264, 4368, 1610, 4194, 4474, 4013, 4266, 1759, 1922, 3663, 1642, 1185, 4356, 3932, 1233, 4212, 3858, 1198, 4192, 3790, 3712, 1969, 934, 1790, 1663, 1336, 3894, 1554, 1494, 1405, 3576, 4314, 3695, 3845, 4511, 3656, 1406, 1596, 4504, 1359, 1734, 3650, 1305, 1208, 1988, 4451, 1282, 4380, 1658, 1452, 1687, 3791, 4491, 4323, 4014, 1304, 1440, 4422, 3861, 1383, 3644, 3691, 4259, 1749, 4216, 1924, 1421, 1387, 3992, 4423, 4253, 1618, 1871, 1256, 3748, 1297, 1586, 1251, 1299, 1954, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 3627, 3777, 1371, 3685, 1325, 1940, 4482, 4258, 3812, 1581, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4153, 2707, 3730, 4426, 4180, 4041, 3923, 4444, 3851, 4429, 4361, 1441, 4394, 4302, 3758, 4510, 3742, 1666, 1407, 4187, 1188, 1724, 1646, 4280, 1613, 4006, 1984, 1351, 3653, 3833, 2561, 4442, 3900, 4298, 1203, 4441, 3628, 3725, 937, 4447, 3776, 3658, 4064, 1324, 1367, 3594, 3680, 1300, 1987, 1862, 3998, 1380, 4185, 3686, 3696, 4463, 1350, 4008, 4230, 3853, 4244, 4083, 1546, 3613, 3273, 3452, 3525, 4105, 3835, 1263, 4020, 4144, 1224, 1199, 3667, 2778, 4412, 4407, 1812, 1810, 3823, 1416, 4333, 3146, 4281, 4211, 1979, 1456, 1239, 4167, 3740, 1876, 1388, 1967, 1474, 1471, 4176, 1232, 1989, 4125, 1741, 3585, 1174, 1396, 4061, 4015, 1207, 3698, 3825, 3922, 4021, 4181, 1598, 1916, 1429, 4502, 4231, 1321, 4390, 3693, 3734, 3635, 1768, 4025, 3976, 3840, 3963, 4286, 1502, 3836, 4161, 3659, 3743, 4130, 3605, 1385, 1783, 4392, 3826, 1473, 1883, 1834, 3984, 4495, 3910, 4160, 3671, 4184, 4132, 4250, 1503, 1237, 1523, 1417, 4063, 1562, 3977, 1971, 1707, 3769, 4074, 1534, 4239, 4003, 3915, 1689, 1776, 1410, 1823, 1498, 1329, 386, 3936, 4054, 4260, 4359, 4131, 4297, 4357, 4022, 3770, 4046, 1222, 3953, 4228, 4226, 4277, 1520, 1767, 1466, 1194, 4137, 1908, 1927, 1758, 3772, 3661, 4186, 4174, 1170, 1859, 4183, 1408, 1403, 941, 4033, 3623, 1826, 3719, 1931, 3584, 4004, 3591, 3967, 4168, 3957, 3878, 4165, 4191, 3980, 4058, 4200, 4336, 4322, 3991, 4106, 1641, 1570, 3978, 4154, 1365, 4257, 3846, 4345, 4379, 3603, 1693, 3937, 3801, 4201, 1898, 1845, 1226, 1220, 3745, 3988, 3975, 4236, 4075, 4146, 4256, 4308, 4000, 4148, 3588, 3868, 4403, 3883, 4326, 4393, 3895, 4237, 3955, 3727, 4005, 4489, 3982, 4437, 3597, 4342, 3872, 3987, 4490, 3876, 3668, 4300, 4042, 3648, 3996, 3800, 4367, 4099, 1807, 3887, 4428, 1884, 1682, 4364, 4306, 3869, 3600, 3914, 1968, 1670, 3811, 1865, 3961, 1506, 4221, 1376, 1379, 3768, 4225, 945, 394, 1714, 1422, 3618, 4433, 1965, 4471, 954, 4139, 1482, 3721, 1825, 1698, 1519, 1352, 4202, 4409, 1977, 1917, 405, 406, 3889, 413, 962, 418, 3675, 3974, 964, 420, 967, 968, 1897, 1272, 4133, 425, 4141, 430, 3749, 4044, 3590, 1944, 4126, 977, 4400, 1606, 980, 2604, 2895, 3448, 4563, 1692, 4055, 4358, 1664, 4309, 4045, 3854, 3682, 2688, 4366, 3425, 3231, 3649, 2759, 2724, 3873, 3755, 1216, 2754, 2606, 3471, 3234, 2565, 2803, 2562, 2684, 3411, 3405, 2690, 1255, 3985, 3535, 1306, 4084, 3282, 447, 1353, 1780, 449, 1485, 453, 1728, 454, 1696, 455, 3116, 1869, 457, 1386, 3879, 2698, 459, 1447, 4425, 1645, 467, 1488, 478, 1604, 1011, 4313, 4073, 4274, 1775, 2000, 2480, 1882, 1817, 1880, 1800, 4473, 3783, 3870, 1948, 3699, 1735, 3792, 4290, 492, 4090, 1019, 1803, 3642, 497, 500, 1333, 1890, 1024, 3608, 1864, 1531, 3636, 1699, 1028, 1366, 1030, 4027, 3478, 3763, 2959, 1293, 517, 3197, 1595, 3413, 2238, 1031, 1448, 1032, 1675, 1797, 3759, 1215, 4122, 3966, 528, 3911, 3289, 1821, 1874, 1037, 536, 1039, 3767, 3683, 540, 541, 1338, 1616, 1901, 1517, 2719, 3803, 4123, 4233, 3129, 3087, 3505, 4158, 1044, 3720, 4351, 4414, 1673, 3453, 3254, 1649, 543, 4240, 3580, 2598, 4062, 2841, 1049, 3515, 1433, 1323, 4301, 1051, 3738, 1270, 3857, 1056, 3662, 1057, 3981, 1063, 568, 570, 3620, 1064, 4002, 3964, 3707, 3666, 1348, 4273, 4497, 1401, 4438, 1895, 1912, 3939, 4170, 4056, 1221, 1654, 1067, 3934, 4080, 4352, 574, 3739, 1565, 3950, 1397, 3716, 1569, 1465, 4268, 1183, 3726, 3881, 4134, 4347, 4012, 3938, 4066, 4198, 4416, 4164, 3630, 4325, 4375, 4371, 4432, 4215, 1753, 3944, 1409, 4195, 4455, 4512, 3750, 3633, 4070, 4104, 4162, 4235, 4346, 1572, 4443, 3761, 1346, 3592, 1891, 4469, 1275, 577, 1963, 4129, 1296, 1360, 3733, 4111, 3993, 4108, 3949, 1556, 1435, 1424, 1830, 1197, 1254, 4120, 1545, 4417, 1469, 1292, 4293, 1827, 3839, 3983, 4395, 4295, 3715, 3855, 3609, 4076, 4175, 3927, 1650, 4299, 1773, 2004, 1267, 1364, 4007, 3729, 3665, 3722, 1712, 1751, 3924, 3952, 1343, 4114, 1704, 4448, 1786, 3354, 586, 3432, 2891, 587, 1903, 2077, 1080, 1563, 2274, 595, 2559, 2590, 2738, 4478, 4069, 1327, 4150, 3260, 3481, 3672, 3625, 2008, 4439, 3852, 3997, 1411, 1268, 4050, 3377, 4424, 1496, 4143, 4493, 3492, 1962, 1412, 4017, 4477, 1794, 4263, 3072, 2199, 3888, 604, 605, 1368, 3827, 4103, 607, 1571, 1438, 3701, 4389, 4897, 1603, 2154, 1715, 4369, 1652, 1733, 4218, 1798, 621, 1667, 1384, 3700, 1788, 1600, 1102, 1423, 2771, 1339, 3070, 2557, 2795, 2906, 3299, 3118, 4779, 2028, 1614, 1947, 630, 4479, 3766, 1106, 634, 1108, 635, 638, 3706, 3735, 3390, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 1269, 3095, 1286, 3624, 4481, 3511, 2735, 4381, 3295, 1585, 3221, 2643, 2739, 3920, 1801, 4279, 2502, 1117, 2965, 2695, 3293, 3264, 2076, 657, 2910, 3391, 2808, 3099, 2913, 3676, 2852, 3284, 2731, 1609, 3537, 3994, 1319, 4467, 1802, 1227, 1623, 667, 1133, 1134, 673, 1933, 1731, 1793, 3342, 683, 1146, 687, 3822, 1601, 692, 3905, 1414, 696, 1695, 4115, 701, 1544, 702, 703, 4031, 2010, 3810, 4452, 3677, 4227, 4402, 4157, 4197, 1337, 1253, 1532, 1493, 1854, 1510, 705, 1153, 4476, 1375, 712, 1245, 715, 4234, 1154, 1314, 4096, 720, 1311, 721, 722, 4217, 726, 3815, 2005, 1964, 1262, 4480, 4294, 1582, 3439, 2833, 4287, 4509, 3272, 4112, 2967, 3651, 1991, 1805, 1628, 3705, 1195, 3859, 3807, 4307, 1454, or 1781.

A VP capsid polypeptide may have a 581 to 589 region having a sequence of SEQ ID NO: 5014, wherein X⁵ of SEQ ID NO: 5014 is K or R. In some embodiments, the 581 to 589 region has a sequence of any one of SEQ ID NOs: 2498, 4177, 742, 16, 17, 18, 745, 3477, 1548, 2982, 3426, 3073, 3169, 747, 3362, 4942, 23, 28, 2038, 3660, 750, 31, 32, 1640, 34, 37, 4925, 1205, 4288, 4053, 3263, 2553, 3947, 2710, 2608, 3467, 2796, 2961, 2529, 3320, 2674, 2635, 3446, 3053, 3688, 4320, 3708, 2807, 3562, 3274, 3374, 3013, 3416, 2981, 3065, 2777, 3469, 2977, 2638, 2972, 3059, 2574, 3380, 4262, 3167, 3368, 1225, 3178, 3006, 3252, 2991, 2642, 2519, 3363, 2633, 42, 755, 44, 756, 46, 1952, 758, 2410, 2551, 3718, 1308, 1230, 3960, 1378, 2589, 3681, 760, 53, 4951, 4591, 762, 3925, 55, 3654, 763, 60, 61, 4086, 1740, 766, 1966, 63, 2041, 64, 769, 770, 2384, 771, 65, 772, 2197, 68, 3816, 69, 2599, 70, 3865, 1549, 3882, 75, 4501, 76, 774, 1413, 776, 3757, 3593, 4043, 4397, 4415, 3754, 1470, 4360, 3945, 2854, 3523, 3669, 1356, 81, 82, 4065, 83, 84, 1294, 87, 3891, 4456, 4040, 1189, 1490, 90, 92, 782, 94, 1831, 1867, 4458, 1936, 100, 792, 2007, 4556, 795, 104, 106, 1849, 2509, 110, 1906, 798, 2359, 2461, 4864, 1344, 116, 120, 121, 803, 1463, 805, 1760, 123, 124, 806, 127, 2085, 130, 132, 809, 1978, 134, 813, 139, 815, 1326, 141, 142, 145, 4816, 1302, 817, 146, 818, 148, 149, 4546, 150, 153, 156, 157, 163, 164, 166, 169, 823, 824, 1483, 175, 177, 178, 179, 826, 827, 182, 183, 829, 184, 185, 190, 191, 192, 2361, 195, 1391, 197, 2223, 201, 202, 4775, 1334, 837, 3901, 3312, 212, 4386, 3028, 213, 1446, 214, 215, 1896, 3490, 217, 219, 223, 225, 2088, 4542, 226, 227, 230, 843, 844, 237, 845, 239, 849, 852, 1536, 854, 244, 245, 248, 855, 251, 253, 255, 257, 858, 860, 262, 863, 4535, 865, 264, 1241, 265, 1477, 267, 867, 1252, 273, 275, 1980, 277, 281, 1892, 879, 1539, 881, 1295, 282, 285, 1626, 2082, 1374, 1620, 4564, 289, 887, 888, 889, 4324, 293, 4138, 296, 3903, 892, 3829, 298, 301, 302, 1589, 4370, 2342, 309, 4059, 310, 1779, 1558, 1552, 317, 320, 898, 321, 2016, 2134, 324, 325, 327, 1635, 4484, 1756, 4609, 332, 4635, 3069, 2637, 3025, 2782, 3247, 3350, 2692, 2586, 3214, 3163, 2541, 3090, 3097, 3462, 3037, 3419, 2780, 2931, 1276, 2789, 3228, 2657, 3189, 2975, 3346, 3067, 2728, 2955, 3408, 3027, 3094, 3203, 2988, 2751, 2953, 2566, 2578, 2901, 2837, 2822, 2745, 3473, 3530, 3040, 2597, 3509, 3522, 3161, 2926, 3209, 3105, 3400, 2630, 2905, 2671, 2818, 2806, 2534, 2773, 2623, 3491, 3243, 3136, 2693, 3164, 2679, 3510, 3110, 2672, 3442, 2929, 3184, 3488, 3102, 3466, 3461, 2788, 2660, 3344, 2720, 3379, 2826, 3249, 3527, 4460, 3352, 4319, 3906, 4248, 3804, 1363, 1404, 4349, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3782, 3629, 3578, 3885, 4081, 1764, 1852, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 4222, 1504, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3909, 4269, 3019, 1415, 3020, 2748, 2646, 2787, 2760, 3158, 3170, 2703, 3494, 3404, 2675, 2749, 3341, 3355, 2594, 3495, 2884, 3516, 3042, 2737, 2923, 3563, 2855, 3476, 2611, 2937, 3383, 2875, 3223, 2878, 1808, 2951, 3519, 2579, 2853, 3387, 3401, 3007, 3568, 3152, 3367, 3358, 336, 3290, 3550, 3309, 2613, 2765, 1355, 2881, 3232, 3458, 3326, 3353, 2927, 3212, 2950, 3376, 3208, 3062, 3612, 3195, 3385, 2686, 1622, 3361, 3022, 3427, 3107, 3140, 3371, 3276, 2831, 3501, 2924, 3366, 3199, 3101, 3321, 3499, 2917, 2683, 1464, 3560, 3236, 2730, 3572, 1607, 909, 2577, 2567, 2532, 3415, 2934, 2984, 2568, 3002, 2338, 4472, 1472, 1529, 4507, 1583, 340, 912, 2013, 913, 2277, 1594, 4440, 917, 343, 344, 345, 346, 350, 351, 4498, 3637, 3570, 3429, 4091, 4338, 3205, 2880, 3261, 3968, 4291, 4204, 4034, 2820, 3912, 3287, 1218, 1190, 4145, 354, 3610, 4224, 1840, 3736, 1770, 4155, 932, 363, 1279, 2156, 1722, 368, 1566, 370, 4967, 372, 1211, 2494, 1726, 1748, 1191, 4039, 4411, 4506, 4270, 4156, 3780, 1550, 3899, 1181, 3919, 1639, 3664, 4189, 4001, 3799, 1240, 1686, 1479, 1307, 1575, 4296, 1665, 3871, 1605, 4408, 4149, 3741, 3893, 1280, 1983, 3979, 3817, 1489, 4214, 3902, 1857, 1904, 3820, 3789, 4252, 4330, 4082, 3785, 3687, 4243, 3704, 4172, 4315, 1192, 2769, 2678, 3296, 2343, 4093, 4453, 3940, 3778, 4374, 4339, 4368, 4194, 4474, 4013, 1771, 1759, 3860, 3832, 1381, 4356, 1233, 4212, 3858, 1198, 3611, 1919, 1819, 3790, 1969, 4247, 1790, 3894, 1554, 3576, 4314, 1851, 1850, 4511, 3656, 3638, 1406, 1443, 1661, 4504, 3831, 4435, 1208, 1988, 3849, 1835, 4380, 4024, 4491, 4323, 4014, 1511, 1981, 1676, 1547, 1383, 3644, 3691, 4259, 1176, 4216, 1924, 1387, 4423, 1256, 4119, 1251, 3990, 4029, 4097, 3890, 3796, 4343, 1202, 4413, 4384, 3808, 3892, 3926, 3756, 4405, 4109, 1210, 4492, 3969, 4436, 3175, 4128, 2970, 1228, 3534, 3437, 3784, 4470, 4010, 4334, 3821, 4171, 3824, 3631, 4494, 1320, 1204, 1223, 1284, 1685, 3760, 3753, 1178, 3884, 2783, 4038, 3545, 2916, 3112, 2834, 3880, 3673, 4102, 4100, 4327, 3634, 3617, 4445, 4303, 1370, 3838, 1187, 3615, 3746, 3935, 1941, 4419, 1171, 3670, 3777, 936, 3685, 1325, 1940, 4482, 4258, 3812, 4124, 4508, 4500, 3652, 3972, 1630, 4421, 1172, 4026, 2740, 3125, 3546, 4513, 4340, 1763, 1487, 3589, 3907, 4041, 3851, 4077, 4187, 1193, 1196, 1724, 1717, 1200, 1975, 1613, 4006, 3653, 3833, 1593, 4442, 3900, 4388, 1820, 4317, 4249, 3628, 3794, 4447, 3776, 3658, 1377, 4011, 4272, 3594, 4185, 3686, 3696, 1350, 4230, 3853, 4244, 1909, 3897, 1546, 3273, 3452, 3525, 4105, 3835, 383, 1263, 1492, 3856, 3586, 1175, 4144, 1224, 1199, 3667, 2778, 4412, 1507, 3146, 4281, 1179, 3843, 3740, 1388, 1474, 1342, 1471, 4176, 1741, 1508, 4378, 1212, 4015, 1207, 3698, 3825, 4231, 3806, 3896, 3963, 4286, 3743, 4130, 3605, 4173, 4265, 1246, 1737, 3826, 1473, 1992, 1883, 1656, 3671, 4063, 1847, 4239, 3915, 1265, 1498, 1329, 3874, 4261, 1791, 3579, 4359, 4131, 4297, 4357, 4046, 3788, 4169, 1925, 4228, 4101, 1889, 1369, 4088, 4019, 1194, 3772, 3850, 1315, 1530, 1859, 4183, 1408, 4033, 3623, 1928, 1459, 1765, 4004, 3591, 3967, 4168, 3957, 3878, 3980, 3958, 4051, 3798, 4032, 3991, 4106, 1641, 4464, 3813, 4257, 3846, 4345, 1787, 1214, 4483, 4201, 1220, 3867, 3640, 3916, 1209, 4236, 4075, 4146, 4256, 4308, 4000, 4148, 3588, 3883, 4326, 4092, 4283, 1553, 3805, 4151, 1281, 1681, 4489, 3982, 4437, 3597, 4342, 3987, 3583, 3647, 389, 4304, 3793, 1937, 3996, 3800, 4367, 4099, 1390, 4364, 4306, 1278, 1672, 1542, 1915, 3600, 3948, 3811, 1362, 4276, 1445, 1721, 1632, 3607, 1317, 1778, 1668, 391, 4406, 4341, 1480, 393, 394, 395, 946, 3618, 4433, 1177, 947, 950, 400, 4471, 401, 402, 954, 3602, 4434, 3721, 1825, 4202, 4409, 406, 1631, 1684, 3889, 410, 412, 2352, 1905, 417, 418, 3675, 1833, 3974, 419, 964, 420, 421, 969, 4733, 1272, 971, 423, 3830, 973, 426, 1708, 428, 4141, 3749, 1619, 975, 4044, 3590, 1706, 4400, 979, 2895, 3448, 438, 982, 2433, 983, 442, 3862, 2435, 443, 4309, 4045, 3854, 3682, 444, 445, 4366, 3425, 3231, 3649, 2759, 2724, 3873, 3599, 3775, 3471, 3234, 2565, 2803, 2562, 2684, 3411, 3405, 2690, 3694, 446, 3985, 3535, 4084, 3282, 988, 2107, 990, 451, 452, 453, 992, 993, 1696, 996, 3116, 1386, 999, 458, 3879, 460, 461, 462, 2339, 1003, 4425, 1645, 1004, 466, 2328, 468, 1231, 1468, 2496, 473, 474, 2108, 1273, 1007, 1744, 1659, 478, 1009, 1012, 4313, 482, 1499, 484, 2513, 2205, 4592, 1458, 4274, 2122, 4905, 4205, 2000, 488, 1894, 3917, 3616, 4311, 4085, 4401, 1310, 3783, 1538, 3870, 1948, 3699, 1735, 3792, 2202, 4090, 2378, 2012, 3642, 1020, 2302, 4665, 4781, 500, 1022, 1023, 4686, 505, 507, 508, 509, 1025, 1732, 1816, 3636, 1028, 516, 1366, 4572, 2489, 1261, 4027, 3763, 2959, 1293, 3197, 1448, 1032, 519, 2420, 522, 3759, 1215, 4122, 1730, 524, 526, 3966, 1035, 527, 528, 529, 530, 531, 1036, 532, 533, 536, 537, 3767, 1929, 1041, 3683, 1043, 4906, 542, 1338, 1796, 4331, 4332, 4466, 3803, 3087, 3505, 1449, 1044, 4414, 3453, 3254, 544, 546, 1045, 547, 4240, 3580, 4680, 549, 550, 2598, 551, 3819, 1259, 4062, 554, 1049, 1050, 1433, 555, 1052, 1053, 3738, 3857, 2089, 1056, 2193, 1257, 1238, 3662, 1059, 1060, 4203, 3981, 1303, 4116, 567, 569, 3620, 4002, 3679, 3707, 4497, 4438, 1264, 3939, 4170, 4056, 1271, 1451, 1654, 3934, 4080, 4352, 574, 3739, 1565, 1419, 3950, 3716, 1465, 3726, 3881, 4134, 4347, 4012, 3938, 4066, 4164, 3630, 4229, 4292, 4215, 1729, 4223, 4455, 4512, 3633, 4070, 4104, 4162, 4443, 3761, 1587, 3592, 1891, 4469, 1963, 4129, 1360, 3949, 1861, 4120, 1292, 4293, 3839, 3983, 3928, 4295, 3715, 3855, 3609, 1990, 4007, 3722, 1624, 3924, 3952, 580, 2003, 1343, 582, 4114, 1818, 1704, 4448, 584, 1078, 1786, 585, 1832, 586, 2891, 587, 4936, 1082, 4459, 2113, 590, 2308, 593, 1858, 597, 2738, 4150, 3260, 3481, 4439, 1268, 4050, 3377, 4424, 3492, 1412, 4477, 4263, 3072, 2009, 3888, 3827, 4103, 1438, 609, 610, 611, 3701, 4389, 613, 4369, 617, 1921, 1373, 1870, 4218, 1099, 1702, 4801, 623, 3700, 1788, 625, 4878, 1423, 1339, 3875, 628, 2557, 2906, 2029, 4876, 1103, 631, 4479, 3766, 632, 1104, 1105, 634, 4651, 1108, 1813, 636, 638, 641, 4581, 1112, 2164, 3735, 3390, 2558, 3314, 3086, 3029, 2956, 3517, 2651, 2914, 2680, 2632, 3121, 3100, 3528, 3526, 3084, 4220, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 4398, 1269, 3624, 4481, 1432, 3295, 1568, 1766, 2643, 2739, 4760, 1115, 3920, 644, 645, 646, 4279, 3762, 1637, 1509, 2200, 1117, 4780, 2965, 2695, 650, 651, 653, 654, 655, 658, 4035, 2910, 3391, 2808, 3676, 2852, 3284, 3537, 1319, 4467, 1802, 1227, 660, 4812, 2364, 1124, 1125, 665, 4917, 1127, 1129, 1132, 1133, 2236, 673, 4557, 1136, 1139, 678, 4737, 4531, 679, 680, 1144, 681, 4525, 2415, 685, 2445, 3822, 690, 691, 1601, 4621, 3905, 695, 1149, 696, 2027, 2160, 1795, 1372, 4115, 2194, 1920, 4031, 1393, 1277, 1400, 4402, 4157, 4028, 704, 1902, 1510, 4182, 1153, 4476, 2053, 710, 1375, 715, 4096, 717, 1155, 719, 1311, 722, 4217, 1156, 723, 1157, 1627, 4566, 1158, 724, 725, 1953, 1201, 3787, 3747, 1846, 3815, 1822, 1537, 1318, 4294, 733, 734, 3439, 2833, 4505, 4267, 3697, 4112, 2967, 3651, 1805, 1163, 3859, 2275, 737, 1167, 1745, 3807, or 4307.

A VP capsid polypeptide may have a 581 to 589 region having a sequence of SEQ ID NO: 5014, wherein X⁶ of SEQ ID NO: 5014 is K or R. In some embodiments, the 581 to 589 region has a sequence of any one of SEQ ID NOs: 738, 4177, 1289, 740, 14, 743, 744, 17, 3477, 1548, 3051, 2982, 2877, 746, 19, 3036, 3426, 3073, 3169, 747, 3362, 2784, 21, 3192, 22, 1638, 748, 24, 749, 27, 1703, 1836, 28, 3660, 29, 750, 1640, 33, 35, 36, 1653, 1205, 3795, 753, 4288, 4219, 2770, 2962, 4241, 4107, 2718, 3317, 4391, 3263, 2553, 3947, 2710, 2608, 3467, 2796, 2961, 2529, 3320, 2674, 2635, 3446, 3053, 3250, 3688, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 1467, 3327, 3178, 3006, 3252, 2991, 3470, 2888, 2572, 3418, 2642, 2519, 3363, 40, 2633, 44, 45, 756, 47, 48, 1952, 3122, 2551, 3718, 3484, 3577, 1308, 3960, 1378, 2994, 3080, 2589, 3681, 51, 760, 762, 3925, 3654, 763, 59, 60, 4086, 1740, 1660, 62, 1966, 63, 2438, 768, 66, 67, 68, 3816, 2599, 2706, 72, 73, 74, 3865, 1549, 1361, 3882, 773, 3713, 1942, 1312, 4501, 76, 1413, 775, 1560, 4289, 3757, 3593, 3908, 4397, 4415, 3723, 3754, 4360, 3945, 2854, 3523, 3669, 1356, 4065, 1294, 1484, 85, 86, 3891, 4456, 4040, 4496, 1982, 2001, 1478, 1189, 1490, 1885, 1926, 89, 780, 1180, 92, 781, 782, 783, 786, 94, 95, 788, 1831, 1867, 96, 4458, 97, 1970, 1936, 1590, 98, 99, 789, 790, 1298, 1688, 791, 792, 2007, 102, 103, 104, 105, 107, 108, 1579, 1906, 111, 798, 112, 799, 800, 1444, 801, 114, 115, 1344, 116, 802, 117, 118, 119, 121, 803, 1463, 1760, 806, 126, 128, 807, 129, 131, 808, 133, 1978, 811, 812, 135, 136, 137, 138, 814, 140, 1326, 141, 142, 143, 2344, 816, 1634, 1302, 817, 818, 147, 148, 149, 150, 151, 154, 155, 819, 820, 156, 157, 158, 161, 162, 165, 822, 168, 169, 170, 823, 172, 1651, 173, 1483, 174, 825, 176, 178, 180, 827, 181, 828, 4911, 184, 186, 188, 189, 830, 192, 193, 194, 196, 1391, 198, 199, 200, 201, 202, 203, 205, 832, 206, 834, 207, 208, 209, 210, 835, 836, 1334, 3901, 3312, 2649, 1450, 4386, 3028, 1446, 1522, 3490, 217, 219, 1612, 220, 221, 222, 839, 223, 840, 224, 225, 228, 229, 230, 231, 842, 232, 233, 234, 238, 847, 239, 850, 851, 853, 1536, 240, 241, 242, 243, 244, 245, 247, 250, 251, 254, 255, 857, 256, 258, 259, 859, 260, 861, 862, 864, 263, 2318, 865, 2326, 264, 1241, 266, 1477, 867, 268, 868, 269, 270, 869, 870, 271, 871, 872, 272, 873, 1252, 1591, 875, 274, 275, 276, 876, 877, 277, 278, 280, 1892, 1539, 881, 3601, 3877, 1460, 282, 1799, 284, 1626, 286, 1374, 1620, 1290, 1354, 887, 1956, 889, 292, 4324, 4138, 891, 296, 3903, 297, 3829, 3809, 4271, 3844, 1436, 300, 1617, 1961, 302, 304, 305, 2147, 1589, 4370, 307, 3646, 4059, 1431, 1331, 312, 1779, 313, 896, 1558, 1552, 318, 319, 322, 1958, 323, 324, 901, 1491, 328, 329, 4484, 907, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3414, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 4248, 1557, 3804, 1602, 4188, 1330, 4349, 3337, 3533, 2629, 3038, 2539, 3567, 3291, 3348, 3115, 3359, 2993, 3079, 3506, 2527, 2963, 2938, 2516, 3113, 2639, 3143, 3044, 2747, 3154, 3280, 3039, 2743, 2902, 3056, 2554, 2522, 3103, 3210, 3434, 2656, 4251, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 2869, 2610, 3298, 2775, 3108, 2603, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 2584, 2712, 3213, 3451, 2634, 3150, 2810, 3303, 2573, 3460, 2615, 3394, 2828, 3329, 4377, 4499, 3555, 2974, 3629, 3578, 3885, 2817, 3248, 2954, 3334, 3571, 3345, 3551, 3384, 3422, 2530, 2973, 4418, 4081, 3951, 1764, 1671, 1843, 3449, 3185, 3399, 2786, 3055, 3472, 3397, 2601, 3564, 3277, 3548, 3179, 3407, 3841, 3347, 3142, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3120, 3286, 2843, 3465, 3946, 2682, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 4269, 3487, 3489, 3019, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1464, 1886, 2666, 3246, 3130, 3560, 3236, 2730, 3572, 3689, 2968, 1457, 3450, 2577, 2567, 2532, 3415, 2934, 2984, 2568, 4147, 3002, 2986, 910, 2979, 2542, 1762, 4472, 1529, 4057, 1976, 4507, 1583, 1711, 2013, 1594, 4440, 344, 920, 921, 4498, 3728, 3637, 3570, 3429, 4091, 2870, 2995, 4338, 3205, 2880, 3261, 3968, 4291, 4204, 4071, 4034, 1742, 2820, 3912, 3287, 1218, 1190, 4145, 354, 1782, 927, 3610, 356, 4224, 1918, 1959, 1840, 3736, 1770, 929, 930, 931, 4155, 360, 361, 362, 364, 1279, 365, 1543, 366, 367, 4049, 1633, 1722, 369, 1495, 1566, 373, 1211, 1726, 1442, 1748, 4142, 1434, 1358, 1191, 3732, 4110, 4039, 4506, 4156, 4036, 4030, 4016, 3899, 1247, 1855, 3919, 1244, 4140, 3930, 4117, 4189, 4001, 3643, 3619, 1240, 1575, 3871, 4135, 4408, 3604, 1274, 4149, 3741, 1938, 3709, 3622, 3834, 1983, 1743, 3979, 4461, 4113, 3817, 4232, 4214, 3902, 1219, 1486, 1512, 1516, 4047, 4082, 4094, 1574, 1248, 4078, 1872, 1426, 3687, 4318, 4243, 3866, 3704, 4172, 1856, 3324, 2769, 2678, 1561, 3296, 377, 1395, 1584, 4093, 4453, 4210, 4009, 4264, 4339, 1738, 4474, 4013, 4266, 1771, 1759, 1922, 3860, 3832, 1349, 1381, 3663, 1185, 3932, 3858, 1198, 4192, 3611, 1919, 3790, 3712, 4095, 1887, 4468, 1720, 4247, 934, 1790, 2002, 1657, 1336, 3894, 3576, 4314, 3695, 1850, 4362, 1815, 3638, 4454, 1661, 4504, 1359, 3650, 1305, 1648, 3831, 4435, 4451, 1993, 3849, 1718, 4068, 1835, 1425, 1428, 4380, 1658, 1452, 4024, 4072, 3791, 4491, 4323, 4014, 1304, 1511, 1981, 1676, 4422, 1283, 3861, 3644, 4259, 1418, 1176, 4216, 1421, 3992, 4423, 4253, 3748, 4119, 1297, 1954, 4450, 3711, 4344, 1578, 4384, 3892, 3674, 3595, 4405, 3973, 4109, 4159, 3744, 4387, 4275, 4492, 4365, 4118, 4278, 3751, 3678, 4353, 3175, 2970, 1228, 3534, 3437, 3406, 3641, 4470, 3941, 4348, 3587, 3818, 4350, 3821, 3929, 3824, 4465, 1398, 3828, 1284, 3773, 4209, 3014, 3884, 2783, 4038, 3545, 2916, 3112, 2834, 3880, 4399, 4048, 3752, 4207, 1235, 3943, 4355, 4087, 4100, 4163, 4486, 4178, 1643, 4152, 4445, 4303, 4284, 1213, 4382, 1399, 3714, 4487, 3615, 4018, 4420, 4121, 3724, 4282, 4310, 4488, 4127, 3670, 4052, 3627, 3777, 1371, 3685, 4258, 3812, 1581, 4124, 4508, 4500, 3652, 3972, 1528, 3717, 2740, 3125, 3546, 3842, 4404, 3690, 3962, 4136, 3589, 3907, 4153, 2707, 4180, 3923, 4444, 4361, 4394, 3758, 4510, 3742, 1710, 1407, 4187, 1193, 1188, 1196, 1724, 1717, 1200, 1997, 1701, 4280, 4006, 1984, 3833, 2561, 4442, 3900, 4298, 1761, 4388, 1597, 4317, 4249, 1341, 1203, 1680, 4441, 3628, 3725, 1945, 937, 4206, 1588, 1439, 3794, 4447, 3776, 3658, 4064, 4011, 4272, 4503, 1324, 3594, 3680, 1300, 1750, 3913, 3774, 1877, 1987, 3998, 3686, 3696, 4463, 3853, 4244, 4083, 1909, 3897, 1546, 3613, 3273, 3452, 3525, 1229, 3835, 1234, 3970, 3856, 3586, 1175, 4321, 2778, 4412, 4407, 3823, 1427, 1507, 1878, 4333, 3146, 4281, 4211, 3843, 1677, 1524, 1525, 1974, 1239, 4167, 3740, 1342, 1471, 4176, 1232, 1989, 4125, 1741, 1580, 4060, 1576, 1212, 3921, 1174, 4061, 4021, 3693, 3734, 1382, 3684, 3806, 3635, 3976, 3840, 4286, 3836, 4161, 3659, 3605, 4392, 4328, 1316, 4173, 4265, 1246, 1629, 1829, 3826, 1992, 1656, 1914, 1839, 3984, 3910, 4160, 4184, 4132, 4250, 1792, 3769, 1534, 4003, 1689, 1599, 1265, 1410, 1823, 1498, 1329, 386, 940, 1655, 3874, 4261, 3626, 3579, 1644, 3936, 4054, 4260, 4297, 4357, 4022, 4046, 1222, 1996, 3788, 3953, 4226, 4277, 4101, 3797, 4019, 3771, 3632, 1402, 4137, 3772, 3661, 4186, 4174, 1170, 3850, 1315, 1611, 1859, 4183, 1403, 4033, 3623, 3596, 1461, 1848, 1173, 1928, 1765, 3719, 3584, 4165, 4191, 3980, 4058, 4200, 4336, 3958, 1260, 4051, 3798, 4032, 1774, 4322, 3991, 4106, 3978, 3965, 4464, 3813, 4154, 1365, 3846, 4345, 4379, 3603, 3937, 3801, 1787, 1214, 4483, 4201, 1845, 1220, 4245, 1347, 3867, 1900, 3640, 3916, 4089, 3745, 3975, 4256, 4308, 4000, 3868, 4403, 3883, 4326, 4393, 3895, 4237, 3955, 4005, 4092, 4372, 4283, 3805, 4151, 3982, 3597, 3872, 3987, 4490, 3876, 3668, 4300, 1679, 1242, 3598, 3583, 3647, 4304, 3779, 1907, 4042, 3648, 3996, 3800, 4367, 4099, 1807, 3887, 4428, 1625, 1390, 1884, 1682, 4364, 4306, 3869, 1278, 942, 1505, 1709, 3600, 3914, 3948, 1865, 1716, 3961, 1506, 4221, 1376, 1683, 4276, 1721, 1632, 3607, 1455, 1913, 1497, 1317, 1951, 1778, 3768, 4225, 1668, 390, 4406, 4341, 1480, 1714, 1422, 397, 3618, 4433, 1177, 3898, 398, 950, 400, 1965, 4471, 953, 401, 403, 955, 1482, 3602, 4434, 3721, 1592, 1825, 1698, 1519, 1352, 4409, 1977, 405, 1631, 1684, 409, 3889, 2117, 411, 413, 414, 415, 961, 1905, 416, 963, 3675, 1833, 3974, 965, 970, 1897, 1272, 4133, 3830, 972, 424, 427, 1708, 4141, 3749, 1619, 975, 4044, 3590, 1944, 4126, 1923, 1706, 432, 976, 978, 4400, 1606, 980, 2604, 2895, 436, 3448, 2188, 438, 1692, 442, 3862, 4055, 4358, 2276, 985, 1664, 443, 4309, 4045, 3854, 3682, 1972, 2688, 4366, 3425, 3231, 3649, 2759, 2724, 3755, 1216, 3599, 3775, 2754, 2606, 3471, 3234, 2565, 2803, 2562, 2684, 3411, 3405, 2690, 1255, 3694, 3985, 3535, 1306, 4084, 3282, 1780, 448, 990, 991, 1485, 992, 1728, 993, 994, 1696, 997, 3116, 1869, 456, 1386, 1000, 3879, 2698, 460, 1001, 461, 1447, 1002, 1003, 463, 464, 4425, 465, 1004, 466, 469, 1231, 1468, 470, 1005, 476, 1273, 1744, 1488, 1659, 1604, 479, 1013, 1014, 481, 4313, 1499, 1016, 4073, 1777, 485, 4274, 487, 1775, 4205, 1526, 1697, 2000, 488, 1894, 1882, 1817, 1880, 490, 1018, 3917, 3616, 4311, 4473, 4085, 4401, 3783, 3765, 1538, 3870, 491, 3699, 3792, 4290, 494, 2421, 4090, 2012, 1803, 3642, 1322, 1020, 502, 1333, 504, 1890, 507, 3608, 1732, 1864, 1816, 1531, 513, 3636, 514, 1699, 1027, 1366, 4950, 4027, 3478, 2959, 1293, 3197, 1595, 3413, 518, 1032, 1033, 1675, 1797, 2315, 521, 1999, 3759, 1215, 4122, 1730, 3966, 1035, 4973, 528, 3911, 3289, 1821, 530, 532, 1874, 533, 1038, 538, 539, 3767, 1929, 3683, 1338, 1616, 1901, 1796, 1517, 4331, 4332, 4466, 2719, 4123, 4233, 1824, 3129, 3087, 3505, 4158, 4254, 3720, 4351, 4414, 3453, 3254, 1649, 544, 4240, 3580, 1046, 548, 549, 2598, 552, 553, 3819, 1259, 4062, 554, 2841, 3515, 1433, 1323, 4301, 555, 558, 3738, 1567, 559, 1054, 1270, 3857, 2175, 1257, 1238, 3662, 561, 1058, 563, 1061, 4203, 1899, 3981, 1062, 1303, 4116, 1946, 3620, 1514, 4002, 3679, 3964, 3707, 3666, 1348, 4273, 1804, 4497, 1401, 4438, 1895, 1264, 1912, 573, 3939, 4170, 4056, 1271, 1521, 1221, 1451, 3934, 4080, 4352, 1700, 3739, 1419, 1811, 3950, 1690, 3716, 1569, 1437, 1674, 4337, 1285, 4268, 1183, 3881, 3938, 4198, 4416, 4229, 4292, 4193, 4325, 4375, 4432, 4215, 3944, 4195, 4223, 1540, 4455, 4512, 1723, 3750, 4070, 4104, 4235, 1863, 4346, 1881, 1814, 4443, 3761, 1587, 3592, 576, 4469, 1275, 1950, 4129, 1296, 1551, 3733, 4111, 3993, 4108, 1287, 1713, 1556, 1736, 1995, 1705, 1861, 4120, 4417, 1785, 1236, 4293, 1827, 3839, 3983, 4395, 1957, 3928, 4295, 3715, 3855, 3609, 4076, 4175, 3927, 1757, 4299, 1773, 1364, 1462, 4007, 3729, 3665, 3722, 1691, 1712, 1624, 1751, 1394, 3924, 3952, 581, 2003, 1559, 1250, 1555, 4114, 1818, 1076, 4448, 1078, 1786, 3354, 1832, 3432, 2891, 1903, 4459, 589, 1563, 1084, 591, 592, 594, 595, 1858, 596, 1453, 2559, 2590, 2738, 4478, 4069, 1809, 1327, 4150, 3260, 3481, 3672, 3625, 4439, 3852, 3997, 3847, 4050, 3377, 4424, 4143, 4493, 3492, 1412, 4017, 4477, 1794, 602, 4263, 3072, 2009, 1754, 3888, 603, 604, 1368, 3827, 4103, 1571, 1438, 611, 1093, 612, 1094, 3701, 4389, 614, 615, 1332, 1715, 4369, 1652, 1541, 1921, 1733, 1373, 1870, 619, 4218, 1798, 1702, 1384, 3700, 1788, 1600, 624, 2473, 1423, 2771, 1339, 3875, 3070, 2015, 2557, 2795, 2906, 2103, 3299, 3118, 4576, 1103, 1614, 1947, 631, 4479, 3766, 1107, 635, 1813, 636, 2431, 637, 1109, 639, 3706, 1110, 640, 642, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 1269, 3095, 1286, 3624, 4481, 3511, 2735, 4381, 3295, 1568, 1766, 3221, 2643, 2739, 3920, 647, 1801, 4279, 3762, 1637, 1509, 648, 1117, 1119, 2965, 2695, 3293, 4722, 1121, 653, 1122, 3264, 655, 656, 657, 4035, 2910, 3391, 2808, 3099, 2913, 3676, 2852, 3284, 2731, 3537, 3994, 1319, 4467, 1623, 660, 1123, 664, 1126, 1128, 1129, 1130, 670, 1527, 671, 1772, 1137, 5005, 1933, 1731, 1793, 3342, 1140, 1141, 687, 3822, 1601, 1148, 693, 3905, 694, 1414, 1149, 697, 1795, 1372, 1695, 1866, 1151, 4115, 1719, 1920, 4999, 1544, 703, 4031, 4305, 1636, 3810, 4452, 3677, 4227, 4402, 4157, 4197, 1337, 1253, 4028, 3989, 704, 1493, 1902, 1152, 4182, 707, 708, 709, 4476, 1375, 713, 714, 1841, 4234, 1154, 1314, 4096, 718, 719, 720, 2063, 1311, 721, 4217, 1627, 1158, 1953, 1201, 3787, 729, 3747, 730, 1846, 3815, 1964, 1822, 1262, 4480, 1537, 1291, 1318, 4294, 1582, 3439, 2833, 4287, 4509, 4505, 4267, 3697, 3272, 4112, 2967, 3651, 1805, 3705, 1195, 3859, 1164, 1165, 2351, 2423, 1745, 1973, 3807, 4307, 1169, 1454, or 1781.

A VP capsid polypeptide may have a 581 to 589 region having a sequence of SEQ ID NO: 5014, wherein X⁷ of SEQ ID NO: 5014 is K or R. In some embodiments, the 581 to 589 region has a sequence of any one of SEQ ID NOs: 740, 15, 742, 743, 17, 22, 24, 1836, 32, 36, 2450, 39, 752, 753, 1998, 3059, 2519, 757, 48, 1952, 50, 52, 760, 2389, 54, 2346, 58, 4086, 1660, 1966, 768, 2402, 772, 71, 72, 2453, 3882, 775, 1806, 3669, 1356, 2132, 81, 84, 85, 87, 4040, 88, 1885, 782, 783, 784, 785, 94, 788, 2173, 98, 2007, 102, 113, 799, 4848, 1444, 4821, 2329, 115, 119, 2290, 123, 806, 128, 134, 812, 145, 817, 154, 2149, 821, 167, 171, 4596, 183, 830, 191, 194, 2071, 195, 4920, 831, 2223, 207, 3901, 211, 212, 213, 215, 224, 4782, 845, 846, 849, 850, 854, 247, 248, 2245, 255, 261, 264, 265, 1477, 270, 874, 876, 279, 882, 1930, 285, 286, 886, 2119, 2468, 887, 888, 4138, 297, 3844, 302, 893, 307, 308, 2155, 1431, 2312, 900, 1635, 906, 2799, 2128, 2918, 1363, 4251, 2622, 3333, 3474, 3614, 2732, 3166, 3151, 3088, 2883, 3318, 2645, 2733, 3213, 3782, 1844, 2523, 2520, 2997, 2681, 3035, 3279, 2764, 3480, 2787, 1808, 3101, 3689, 4147, 1762, 1976, 340, 1594, 2439, 348, 4498, 1518, 1782, 926, 357, 359, 932, 363, 369, 1873, 3780, 4140, 4001, 3709, 3834, 1752, 4113, 3817, 4232, 1340, 1489, 4214, 3902, 3820, 4330, 1872, 3687, 4318, 375, 4210, 3860, 4212, 3695, 1851, 4504, 1910, 1428, 4323, 4422, 4259, 1176, 3711, 4365, 4383, 3969, 4128, 3802, 3863, 3821, 4171, 3824, 4462, 3760, 3753, 2783, 3880, 3943, 4163, 4178, 4199, 3724, 3670, 4482, 3692, 4430, 4316, 3645, 3690, 3589, 3730, 4426, 3851, 4429, 1335, 1717, 1997, 1975, 4280, 4006, 3900, 4298, 4388, 1341, 1350, 1909, 4412, 4407, 4281, 4167, 1893, 3585, 4015, 1747, 4286, 3826, 1473, 1914, 3984, 4495, 4184, 3977, 1707, 4074, 4003, 3915, 1410, 1498, 940, 4131, 4357, 1925, 1466, 1611, 1530, 4168, 4245, 4075, 3987, 1679, 4306, 3869, 3768, 393, 2265, 3618, 4433, 947, 399, 401, 402, 4139, 1352, 406, 415, 418, 420, 4919, 431, 4044, 976, 433, 434, 435, 442, 3873, 3411, 3985, 987, 1485, 2262, 457, 458, 4425, 466, 467, 468, 1468, 474, 1008, 1012, 1016, 4518, 1697, 490, 3616, 2247, 2302, 499, 500, 501, 509, 2110, 512, 1797, 520, 525, 3966, 1038, 537, 1041, 3683, 2165, 542, 4331, 3129, 1044, 4351, 3453, 1047, 1323, 4301, 557, 3738, 559, 1270, 3857, 1056, 2186, 1057, 1059, 565, 566, 3981, 567, 3620, 4002, 3666, 3939, 1221, 1397, 1437, 4337, 4268, 3938, 4292, 4215, 3750, 3633, 4070, 3592, 1827, 3839, 4395, 3855, 3665, 1624, 2395, 1818, 586, 587, 1903, 1080, 1081, 1082, 589, 1084, 1085, 594, 596, 4439, 3997, 1962, 1087, 4777, 606, 607, 610, 1094, 4389, 1603, 1095, 616, 618, 622, 1104, 4536, 3706, 2481, 3100, 3200, 3621, 4179, 1432, 4381, 643, 1115, 644, 2307, 2477, 652, 657, 658, 3994, 4467, 661, 663, 665, 4600, 1137, 1933, 4840, 1140, 678, 680, 683, 686, 692, 693, 696, 2081, 2280, 698, 1400, 4197, 1532, 1854, 706, 711, 712, 4570, 714, 1841, 1314, 4096, 2249, 726, 1846, 2385, 1318, 733, 3651, 1165, 4839, 4968, 4684, or 1454.

A VP capsid polypeptide may have a 581 to 589 region having a sequence of SEQ ID NO: 5014, wherein X³ of SEQ ID NO: 5014 is a basic amino acid residue. In some embodiments, the 581 to 589 region has a sequence of any one of SEQ ID NOs: 739, 4177, 1289, 740, 741, 18, 745, 3477, 1548, 3051, 2982, 2877, 3036, 3426, 3073, 3169, 747, 3362, 2784, 21, 3192, 23, 1638, 748, 1703, 1836, 3660, 29, 750, 1640, 33, 34, 35, 36, 37, 38, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 3327, 3178, 3006, 3252, 2991, 3470, 2888, 2572, 3418, 2642, 2519, 3363, 40, 2633, 41, 42, 43, 755, 45, 756, 46, 48, 1952, 758, 49, 3122, 2551, 3718, 3484, 3577, 759, 1230, 3960, 1378, 2994, 3080, 2589, 3681, 52, 760, 53, 762, 4659, 54, 3925, 55, 3654, 56, 60, 61, 4086, 1740, 1660, 765, 766, 62, 1966, 63, 64, 768, 771, 65, 66, 67, 68, 3816, 2599, 2706, 2399, 74, 3865, 1549, 1361, 2453, 3882, 3713, 1942, 75, 1312, 4501, 76, 77, 78, 1560, 4289, 3757, 79, 3593, 4043, 1806, 3908, 4397, 1615, 4415, 3723, 2006, 3754, 1470, 4360, 3945, 2854, 3523, 3669, 82, 4065, 83, 1294, 1484, 3891, 4456, 4040, 4496, 1982, 2001, 1478, 1189, 88, 1490, 1885, 1926, 89, 90, 91, 779, 1180, 92, 785, 786, 94, 95, 1831, 1867, 4458, 97, 1970, 1936, 1590, 99, 100, 789, 790, 1298, 101, 1688, 791, 792, 2007, 103, 794, 795, 104, 105, 106, 1849, 108, 4736, 1579, 110, 1906, 113, 1444, 1344, 116, 802, 117, 118, 2158, 121, 803, 804, 1463, 805, 122, 2058, 124, 125, 127, 4994, 807, 132, 809, 810, 133, 1978, 812, 135, 136, 137, 138, 813, 4673, 140, 815, 1326, 141, 142, 143, 144, 145, 4816, 1634, 1302, 817, 146, 818, 147, 148, 149, 151, 152, 153, 154, 155, 819, 820, 157, 821, 159, 160, 163, 164, 165, 166, 822, 167, 168, 169, 170, 171, 823, 824, 1651, 173, 1483, 174, 175, 825, 176, 177, 180, 826, 827, 181, 828, 182, 183, 4641, 829, 184, 185, 186, 187, 188, 189, 830, 191, 192, 193, 194, 195, 196, 1391, 197, 2223, 200, 201, 202, 203, 204, 205, 832, 833, 206, 834, 835, 836, 1334, 837, 3901, 3312, 2649, 1301, 211, 212, 1450, 4386, 3028, 213, 1446, 214, 215, 1896, 1522, 3490, 838, 2241, 217, 218, 219, 1612, 220, 221, 222, 839, 223, 840, 224, 226, 227, 230, 231, 842, 232, 233, 235, 844, 236, 237, 2150, 851, 852, 853, 1536, 240, 854, 242, 243, 244, 245, 246, 247, 855, 4634, 249, 250, 251, 252, 253, 254, 255, 857, 256, 257, 258, 259, 858, 859, 260, 261, 860, 861, 862, 262, 2326, 264, 1241, 265, 266, 866, 1477, 267, 867, 869, 870, 271, 871, 872, 272, 874, 1252, 273, 1591, 275, 1980, 276, 876, 877, 277, 278, 279, 1892, 879, 1539, 880, 1295, 3601, 3877, 1930, 1420, 1678, 1460, 2306, 282, 1799, 283, 1626, 1374, 287, 1620, 1290, 1354, 888, 291, 1956, 889, 292, 4324, 293, 4138, 891, 2224, 3903, 297, 3829, 3809, 4271, 3844, 1436, 1515, 1617, 1961, 304, 306, 1589, 4370, 308, 309, 894, 3646, 4059, 1431, 1331, 4948, 312, 895, 1779, 313, 314, 315, 316, 896, 1558, 1552, 897, 898, 321, 322, 899, 1958, 2134, 324, 901, 325, 902, 326, 1491, 329, 330, 905, 4484, 1756, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 1888, 4248, 1557, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 3435, 3482, 3020, 2748, 2646, 2787, 2760, 2563, 3085, 3133, 3158, 3170, 2703, 3494, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 2979, 2542, 2492, 1762, 4472, 1472, 1529, 4057, 1976, 4507, 1583, 338, 911, 1711, 340, 912, 2013, 341, 1594, 4440, 917, 344, 918, 919, 921, 922, 347, 349, 351, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 4145, 354, 925, 1782, 926, 927, 3610, 357, 358, 359, 4224, 1918, 1959, 3736, 1770, 931, 4155, 364, 1279, 365, 1543, 2472, 367, 4049, 1633, 1722, 368, 369, 1495, 1566, 370, 1211, 1873, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 3741, 1938, 3709, 1727, 3893, 1280, 3622, 1784, 3834, 1752, 1983, 1743, 3979, 4461, 4113, 3817, 4232, 1340, 1489, 4214, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 377, 1395, 4093, 4453, 3940, 3778, 4374, 4210, 4009, 4264, 4339, 4368, 1738, 378, 1610, 4194, 4474, 4013, 4266, 1771, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 2002, 1657, 1663, 1336, 3894, 1554, 1494, 1405, 3576, 4314, 3695, 1851, 4362, 1815, 3845, 4511, 3656, 3638, 1406, 1596, 4454, 1443, 1661, 1359, 1734, 3650, 1910, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1618, 1871, 1256, 3748, 4119, 1297, 1586, 1251, 1299, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1680, 4441, 3628, 3725, 1945, 937, 4206, 1588, 1439, 3794, 4447, 1838, 3776, 3658, 4064, 1377, 4011, 4272, 4503, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 3613, 3273, 3452, 3525, 1229, 4105, 3835, 383, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 4125, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 1520, 1767, 1466, 1194, 4137, 1908, 1927, 1758, 3772, 3661, 4186, 4174, 1170, 3850, 1315, 1611, 1530, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 1716, 3961, 1506, 4221, 1376, 1683, 4276, 1445, 1721, 1632, 3607, 1328, 1455, 1913, 1497, 1317, 1951, 1778, 1379, 3768, 4225, 1668, 390, 391, 4406, 4341, 1480, 395, 1714, 1422, 396, 2265, 397, 3618, 4433, 1177, 3898, 398, 949, 950, 399, 951, 400, 1965, 2345, 4471, 952, 402, 2475, 403, 404, 4139, 1482, 3602, 4434, 3721, 1592, 1519, 1352, 4202, 4409, 1977, 1917, 407, 408, 1631, 1684, 409, 3889, 958, 959, 412, 960, 414, 415, 961, 1905, 416, 963, 417, 3675, 1833, 3974, 420, 421, 965, 966, 969, 970, 422, 1897, 4754, 423, 4133, 3830, 972, 424, 973, 427, 1708, 4141, 3749, 431, 1619, 975, 4044, 3590, 1944, 4126, 1923, 1706, 4400, 434, 1606, 979, 2604, 2895, 436, 3448, 1692, 983, 3862, 4055, 4358, 984, 985, 1664, 4045, 3854, 3682, 2491, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 3535, 1306, 4084, 3282, 1780, 987, 988, 450, 990, 451, 452, 991, 992, 1728, 994, 995, 454, 1696, 998, 3116, 1869, 456, 1386, 999, 3879, 2698, 1001, 461, 1447, 462, 464, 4425, 1645, 1231, 1468, 472, 473, 4725, 476, 1273, 1007, 1744, 1488, 1659, 477, 1604, 481, 4313, 482, 483, 1499, 484, 2513, 1016, 4073, 1777, 1458, 4274, 4539, 4528, 487, 1775, 4205, 1526, 1697, 1017, 2000, 489, 1882, 1817, 1880, 490, 1018, 3917, 3616, 4311, 1800, 4473, 4085, 4401, 1310, 3783, 3765, 491, 1948, 3699, 1735, 3792, 4290, 495, 4090, 2247, 496, 1019, 2012, 1803, 3642, 1322, 498, 1021, 501, 1022, 502, 1333, 503, 1023, 1890, 1025, 510, 1026, 3608, 1732, 511, 2397, 1864, 1816, 1531, 3636, 1699, 1027, 1029, 1366, 2112, 2124, 2376, 1261, 4027, 3478, 3763, 2959, 517, 3197, 1595, 3413, 1448, 518, 2057, 1675, 1797, 2148, 2420, 520, 2315, 523, 1999, 3759, 1215, 4122, 1730, 524, 526, 3966, 3911, 529, 3289, 1821, 530, 531, 1874, 1038, 536, 537, 3767, 1929, 1041, 1042, 3683, 542, 1338, 1517, 4331, 4332, 4466, 2719, 3803, 4123, 4233, 1824, 3129, 3087, 3505, 4158, 4254, 1449, 3720, 4351, 4414, 1673, 3453, 3254, 1649, 544, 545, 546, 4240, 3580, 1046, 1047, 550, 2598, 551, 552, 3819, 1259, 4062, 2841, 1050, 3515, 4301, 557, 1052, 1053, 3738, 1567, 1270, 3857, 1055, 2198, 1257, 1238, 3662, 561, 562, 1058, 566, 1060, 1061, 2231, 4203, 1899, 3981, 1062, 1303, 4116, 567, 1946, 3620, 1514, 4002, 3679, 3964, 3707, 3666, 1348, 4273, 1804, 4497, 1401, 4438, 1895, 1264, 572, 1912, 3939, 4170, 4056, 1271, 1065, 1521, 1221, 1451, 1654, 3934, 4080, 4352, 1700, 3739, 1565, 1419, 1811, 3950, 1690, 1397, 3716, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 1723, 1070, 3750, 3633, 4070, 4104, 4162, 4235, 1863, 4346, 1881, 1814, 1572, 4443, 3761, 1346, 1587, 1072, 3592, 576, 1891, 4469, 1275, 1950, 1963, 4129, 1296, 1551, 3733, 4111, 3993, 4108, 3949, 1287, 1713, 1736, 1995, 1435, 1424, 1830, 1705, 1861, 1197, 1254, 4120, 1545, 4417, 1469, 1785, 1236, 4293, 3839, 3983, 4395, 1957, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1624, 1751, 1394, 3924, 3952, 580, 581, 2003, 1343, 1559, 1250, 1555, 4114, 1818, 1075, 1076, 1077, 1704, 4448, 1078, 1079, 1786, 585, 3354, 1832, 3432, 2891, 2505, 1903, 1081, 1082, 4459, 1083, 1563, 590, 591, 1085, 594, 597, 598, 1453, 2559, 2590, 2738, 4478, 4069, 1809, 1327, 4150, 3260, 3481, 3672, 3625, 2008, 4439, 3852, 3997, 1411, 599, 3847, 1268, 4050, 3377, 4424, 1496, 4143, 4493, 3492, 1962, 600, 601, 4017, 4477, 1794, 4263, 3072, 2009, 1754, 1087, 1088, 3888, 2324, 605, 1368, 3827, 4103, 606, 4549, 1090, 1571, 1091, 1438, 1092, 1093, 612, 1094, 3701, 4389, 613, 1603, 616, 1332, 1715, 4369, 1652, 1096, 617, 1541, 618, 1921, 1733, 1373, 1870, 619, 4218, 1798, 1099, 1100, 1702, 2239, 1101, 1667, 1384, 623, 3700, 1788, 1600, 624, 626, 2771, 627, 1339, 3875, 628, 629, 3070, 4818, 4860, 2557, 2795, 2906, 3118, 4657, 4646, 4875, 4627, 4747, 1614, 1947, 4618, 4479, 3766, 632, 1104, 1105, 633, 635, 1813, 636, 2213, 3706, 1110, 640, 1111, 641, 642, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 3095, 1286, 3624, 4481, 1432, 3511, 2735, 4381, 3295, 1585, 1114, 1766, 3221, 2643, 2739, 3920, 644, 1116, 645, 646, 2307, 647, 1801, 4279, 3762, 1637, 1509, 1119, 2965, 2369, 2695, 3293, 650, 651, 1120, 652, 1121, 4909, 1122, 3264, 655, 658, 659, 4035, 2910, 3391, 2808, 3099, 2913, 3676, 2852, 3284, 2731, 1609, 3994, 1319, 4467, 1802, 1227, 1623, 661, 2364, 662, 663, 664, 1124, 1125, 1127, 1128, 1129, 668, 669, 1130, 670, 1131, 1527, 1132, 671, 1772, 1135, 672, 5005, 2208, 4952, 4945, 1933, 1731, 1793, 675, 676, 3342, 1138, 1139, 677, 679, 1142, 1143, 681, 1145, 682, 685, 688, 3822, 689, 690, 691, 1147, 1601, 3905, 694, 695, 1414, 4793, 4575, 1150, 1795, 1372, 1695, 1866, 4115, 1719, 698, 2217, 1920, 699, 700, 1544, 702, 703, 4031, 4305, 1636, 1393, 2010, 3810, 4452, 3677, 4227, 1277, 1400, 4402, 4157, 4197, 1337, 1253, 1532, 4028, 3989, 1493, 1854, 1902, 1510, 2340, 4182, 706, 707, 708, 4476, 1245, 714, 1841, 4234, 1314, 4096, 717, 1155, 2249, 4217, 1156, 723, 1157, 1627, 725, 1159, 1953, 1160, 1201, 727, 3787, 4568, 3747, 730, 1846, 3815, 2005, 1964, 1822, 1262, 4480, 1537, 1291, 1318, 4294, 1582, 734, 3439, 2833, 4287, 4509, 4505, 4267, 3697, 3272, 4112, 2967, 3651, 1628, 3705, 1195, 3859, 1164, 2394, 1165, 1166, 1745, 1973, 1168, 3807, 4307, 2303, 1454, or 1781.

A VP capsid polypeptide may have a 581 to 589 region having a sequence of SEQ ID NO: 5014, wherein X⁶ of SEQ ID NO: 5014 is a basic amino acid residue. In some embodiments, the 581 to 589 region has a sequence of any one of SEQ ID NOs: 4884, 738, 4177, 1289, 740, 14, 743, 744, 17, 3477, 1548, 3051, 2982, 2877, 746, 19, 3036, 3426, 3073, 3169, 747, 3362, 2784, 4889, 21, 3192, 4942, 22, 1638, 748, 24, 749, 27, 1703, 1836, 28, 3660, 29, 750, 1640, 33, 35, 36, 2450, 1653, 1205, 3795, 753, 4288, 4219, 2770, 2962, 4241, 4107, 2718, 3317, 4391, 3263, 2553, 3947, 2710, 2608, 3467, 2796, 2961, 2529, 3320, 2674, 2635, 3446, 3053, 3250, 3688, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 1467, 3327, 3178, 3006, 3252, 2991, 3470, 2888, 2572, 3418, 2642, 2519, 3363, 40, 2633, 44, 45, 756, 47, 48, 1952, 3122, 2551, 3718, 3484, 3577, 759, 50, 1308, 3960, 1378, 2994, 3080, 2589, 3681, 51, 760, 2389, 762, 3925, 3654, 763, 4626, 59, 60, 4086, 1740, 1660, 62, 1966, 63, 2438, 768, 66, 67, 2197, 68, 3816, 2599, 2706, 72, 73, 74, 3865, 1549, 1361, 2453, 3882, 773, 3713, 1942, 1312, 4501, 76, 1413, 775, 1560, 4289, 3757, 3593, 3908, 4397, 4415, 3723, 3754, 4360, 3945, 2854, 3523, 3669, 1356, 2132, 4065, 1294, 1484, 85, 86, 3891, 4456, 4040, 4496, 1982, 2001, 1478, 1189, 1490, 1885, 1926, 89, 780, 1180, 92, 781, 782, 783, 2356, 786, 94, 95, 788, 1831, 1867, 96, 4458, 97, 1970, 4656, 1936, 1590, 98, 99, 789, 790, 1298, 101, 1688, 791, 792, 2007, 102, 103, 104, 105, 107, 108, 1579, 110, 1906, 111, 798, 112, 799, 800, 2461, 1444, 801, 114, 115, 1344, 116, 802, 117, 118, 119, 121, 803, 1463, 1760, 124, 806, 126, 128, 807, 129, 131, 808, 2281, 133, 1978, 811, 812, 135, 136, 137, 138, 814, 140, 1326, 141, 142, 143, 2344, 816, 1634, 1302, 817, 818, 147, 148, 149, 150, 151, 154, 155, 819, 820, 156, 157, 2295, 158, 161, 162, 165, 822, 168, 169, 170, 823, 172, 1651, 173, 1483, 174, 825, 176, 178, 180, 827, 181, 828, 4911, 184, 185, 186, 188, 189, 830, 192, 193, 194, 196, 1391, 197, 198, 199, 200, 201, 202, 203, 4775, 205, 832, 206, 834, 207, 208, 209, 210, 835, 836, 1334, 3901, 3312, 2649, 1450, 4386, 3028, 1446, 2206, 215, 1522, 3490, 217, 219, 1612, 220, 221, 222, 839, 223, 840, 224, 225, 4751, 228, 229, 230, 231, 2448, 842, 232, 233, 4782, 234, 235, 238, 847, 239, 850, 851, 853, 1536, 240, 241, 242, 243, 244, 245, 246, 247, 4923, 4696, 250, 251, 254, 255, 857, 256, 258, 259, 859, 260, 861, 862, 4966, 864, 263, 4970, 2318, 865, 2326, 264, 1241, 266, 1477, 867, 268, 868, 269, 270, 869, 870, 271, 871, 872, 272, 873, 4655, 4972, 1252, 1591, 875, 274, 275, 1980, 276, 876, 877, 2066, 277, 278, 280, 2323, 1892, 1539, 2214, 881, 882, 3601, 3877, 1930, 1460, 282, 1799, 284, 4553, 4772, 1626, 286, 1374, 1620, 1290, 1354, 887, 1956, 889, 4765, 292, 4324, 4138, 891, 296, 3903, 297, 3829, 3809, 4271, 3844, 1436, 300, 1617, 1961, 5010, 302, 304, 305, 2147, 1589, 4370, 307, 3646, 4059, 1431, 1331, 312, 1779, 313, 896, 1558, 1552, 318, 319, 322, 1958, 2454, 323, 324, 901, 1491, 328, 329, 330, 4820, 4484, 1756, 907, 4584, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3414, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 4248, 1557, 3804, 1602, 4188, 1330, 4349, 3337, 3533, 2629, 3038, 2539, 3567, 3291, 3348, 3115, 3359, 2993, 3079, 3506, 2527, 2963, 2938, 2516, 3113, 2639, 3143, 3044, 2747, 3154, 3280, 3039, 2743, 2902, 3056, 2554, 2522, 3103, 3210, 3434, 2656, 4251, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 2869, 2610, 3298, 2775, 3108, 2603, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3629, 3578, 3885, 2817, 3248, 2954, 3334, 3571, 3345, 3551, 3384, 3422, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3120, 3286, 2843, 3465, 3946, 4222, 2682, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 4269, 3487, 3489, 3019, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 3689, 2968, 1457, 3450, 2577, 2567, 2532, 3415, 2934, 2984, 2568, 4147, 3002, 2986, 910, 2979, 2542, 1762, 4472, 1529, 4057, 1976, 4507, 1583, 1711, 2013, 1594, 4440, 343, 344, 920, 921, 348, 4498, 3728, 3637, 3570, 3429, 4091, 2870, 2995, 4338, 3205, 2880, 3261, 3968, 4291, 4204, 4071, 4034, 1742, 2820, 3912, 3287, 1218, 1190, 4145, 354, 1782, 927, 3610, 356, 4224, 1918, 1959, 1840, 3736, 1770, 929, 930, 931, 4155, 360, 361, 362, 364, 1279, 365, 1543, 366, 367, 4049, 1633, 1722, 369, 1495, 1566, 373, 1211, 2494, 1726, 1442, 1748, 4142, 1434, 1358, 1191, 3732, 4110, 4039, 4411, 4506, 4156, 4036, 4030, 4016, 3899, 1247, 1855, 3919, 1244, 4140, 3930, 4117, 4189, 4001, 3643, 3619, 1240, 1575, 3871, 4135, 4408, 3604, 1274, 4149, 3741, 1938, 3709, 3622, 3834, 1983, 1743, 3979, 4461, 4113, 3817, 4232, 4214, 3902, 1219, 1486, 1512, 1516, 4047, 4082, 4094, 1574, 1248, 4078, 1872, 1426, 3785, 3687, 4318, 4243, 3866, 3704, 4172, 1513, 1856, 3324, 2769, 2678, 1561, 3296, 2348, 377, 1395, 1584, 4093, 4453, 3778, 4210, 4009, 4264, 4339, 1738, 4194, 4474, 4013, 4266, 1771, 1759, 1922, 3860, 3832, 1349, 1381, 3663, 1185, 3932, 3858, 1198, 4192, 3611, 1919, 3790, 3712, 4095, 1887, 4468, 1720, 4247, 934, 1790, 2002, 1657, 1336, 3894, 1554, 3576, 4314, 3695, 1850, 4362, 1815, 3656, 3638, 4454, 1443, 1661, 4504, 1359, 3650, 1305, 1648, 3831, 4435, 4451, 1993, 3849, 1718, 4068, 1835, 1425, 1428, 4380, 1658, 1452, 4024, 4072, 3791, 4491, 4323, 4014, 1304, 1511, 1981, 1676, 4422, 1283, 3861, 3644, 4259, 1749, 1418, 1176, 4216, 1924, 1421, 3992, 4423, 4253, 3748, 4119, 1297, 1299, 1954, 4450, 3990, 4029, 3711, 4344, 1578, 4384, 3892, 3674, 3595, 4405, 3973, 4109, 4159, 3744, 4387, 1960, 4275, 4492, 4365, 4118, 3655, 3886, 4278, 3751, 3678, 4353, 3175, 2970, 1228, 3534, 3437, 3406, 3641, 3639, 4470, 3941, 4348, 3587, 3818, 3814, 4350, 3821, 3929, 3824, 4465, 4494, 1398, 3828, 1284, 3773, 4209, 3014, 3884, 2783, 4038, 3545, 2916, 3112, 2834, 3880, 4399, 4048, 3752, 4207, 1235, 3943, 4355, 4087, 4100, 4163, 4486, 4178, 1643, 4152, 3634, 4445, 4303, 4284, 1213, 4382, 1399, 3714, 4487, 3615, 4018, 4420, 4121, 3724, 4282, 4310, 4488, 4127, 3670, 4052, 3627, 3777, 1371, 3685, 4482, 4258, 3812, 1581, 4124, 4508, 4500, 3652, 3972, 1309, 1528, 3717, 1217, 2740, 3125, 3546, 3842, 4404, 3690, 3962, 4329, 4410, 3703, 4136, 3589, 3907, 4153, 2707, 4180, 3923, 4444, 4361, 4394, 4302, 3758, 4510, 3742, 1710, 1335, 1666, 1407, 4187, 1193, 1188, 1196, 1724, 1717, 1200, 1997, 1701, 4280, 4006, 1984, 3833, 1593, 2561, 4442, 3900, 4298, 1761, 4388, 1597, 4317, 4249, 1341, 1203, 1680, 4441, 3628, 3725, 1945, 937, 4206, 1588, 1439, 3794, 4447, 3776, 3658, 4064, 4011, 4272, 4503, 1324, 1367, 3594, 3680, 1300, 1750, 3913, 3774, 1877, 1987, 3998, 1380, 4185, 3686, 3696, 4463, 3853, 4244, 4083, 1909, 3897, 1546, 3613, 3273, 3452, 3525, 1229, 3835, 1234, 3970, 3856, 3586, 1175, 4321, 2778, 4412, 4407, 1810, 3823, 1427, 1507, 1878, 4333, 3146, 4281, 4211, 1179, 3843, 1677, 1524, 1525, 1974, 1239, 4167, 3740, 1342, 1471, 4176, 1232, 1989, 4125, 1741, 1580, 4060, 1576, 1212, 3921, 1174, 4061, 4021, 3693, 3734, 1382, 3684, 3806, 3635, 3976, 3840, 4286, 3836, 4161, 3659, 3743, 3605, 4392, 4328, 1316, 4173, 4265, 1246, 1629, 1829, 3826, 1992, 1656, 1837, 1914, 1839, 3984, 3910, 4160, 4184, 4132, 4250, 1562, 1847, 1792, 3769, 1534, 4003, 1689, 1599, 1265, 1410, 1823, 1498, 1329, 386, 940, 1655, 3874, 4261, 3626, 3579, 1644, 3936, 4054, 4260, 4297, 4357, 4022, 4046, 1222, 1996, 3788, 3953, 4226, 4277, 4101, 3797, 4019, 3771, 3632, 1402, 4137, 3772, 3661, 4186, 4174, 1170, 3850, 1315, 1611, 1859, 4183, 1403, 4033, 3623, 3596, 1461, 1848, 1173, 1928, 1765, 3719, 3584, 3591, 4165, 4191, 3980, 4058, 4200, 4336, 3958, 1260, 4051, 3798, 4032, 1774, 4322, 3991, 4106, 3978, 3965, 4464, 3813, 4154, 1365, 3846, 4345, 4379, 3603, 1693, 3937, 3801, 1787, 1214, 4483, 4201, 1898, 1845, 1220, 4245, 1347, 3867, 1900, 3640, 3916, 4089, 3745, 3975, 4256, 4308, 4000, 3868, 4403, 3883, 4326, 4393, 3895, 4237, 3955, 4005, 4092, 4372, 4283, 3805, 4151, 3982, 3597, 3872, 3987, 4490, 3876, 3668, 4300, 1679, 1242, 3598, 3583, 3647, 4304, 3779, 1907, 4042, 3648, 3996, 3800, 4367, 4099, 1807, 3887, 4428, 1625, 1390, 1884, 1682, 4364, 4306, 3869, 1278, 942, 1505, 1542, 1709, 3600, 3914, 3948, 1865, 1716, 3961, 1506, 4221, 1376, 1683, 4276, 1721, 1632, 3607, 1455, 1913, 1497, 1317, 1951, 1778, 3768, 4225, 1668, 390, 4406, 4341, 1480, 392, 1714, 1422, 397, 3618, 4433, 1177, 3898, 398, 950, 400, 1965, 4471, 953, 401, 403, 955, 1482, 3602, 4434, 3721, 1592, 1825, 1698, 1519, 1352, 4409, 1977, 405, 1631, 1684, 409, 3889, 2117, 411, 413, 414, 415, 961, 1905, 416, 963, 3675, 1833, 3974, 965, 970, 1897, 1272, 971, 4133, 3830, 972, 424, 427, 1708, 4141, 3749, 1619, 975, 4044, 3590, 1944, 4126, 1923, 1706, 432, 976, 978, 4400, 433, 1606, 980, 2604, 2895, 436, 3448, 2188, 438, 1692, 982, 2243, 439, 442, 3862, 4055, 4358, 2276, 985, 1664, 443, 4309, 4045, 3854, 3682, 1972, 2688, 4366, 3425, 3231, 3649, 2759, 2724, 3755, 1216, 3599, 3775, 2754, 2606, 3471, 3234, 2565, 2803, 2562, 2684, 3411, 3405, 2690, 1255, 3694, 3985, 3535, 1306, 4084, 3282, 1353, 1780, 448, 2031, 990, 991, 1485, 992, 1728, 993, 4703, 994, 1696, 997, 998, 3116, 1869, 456, 1386, 4778, 2040, 1000, 4981, 3879, 2698, 460, 1001, 461, 1447, 1002, 1003, 463, 464, 4425, 465, 1004, 466, 469, 1231, 1468, 470, 2496, 1005, 476, 1273, 1744, 1488, 1659, 4598, 1604, 479, 1013, 1014, 481, 4313, 1499, 1016, 4073, 1777, 2172, 485, 4274, 4599, 4796, 487, 4710, 1775, 4205, 1526, 1697, 2000, 488, 1894, 1882, 1817, 1880, 490, 1018, 3917, 3616, 4311, 1800, 4473, 4085, 4401, 3783, 3765, 1538, 3870, 491, 3699, 3792, 4290, 2074, 492, 494, 2421, 4090, 2012, 1803, 3642, 1322, 1020, 502, 1333, 504, 4805, 1890, 507, 3608, 1732, 511, 2397, 1864, 1816, 1531, 513, 3636, 514, 1699, 1027, 4987, 1366, 4664, 4950, 4027, 3478, 2959, 1293, 3197, 1595, 3413, 518, 4698, 1032, 1033, 1675, 1797, 4578, 4854, 2315, 521, 1999, 3759, 1215, 4122, 1730, 3966, 1035, 4973, 528, 3911, 3289, 1821, 530, 2426, 532, 1874, 533, 1038, 538, 539, 3767, 1929, 3683, 2100, 4853, 1338, 2021, 1616, 1901, 1796, 1517, 4331, 4332, 4466, 2719, 4123, 4233, 1824, 3129, 3087, 3505, 4158, 4254, 3720, 4351, 4414, 3453, 3254, 1649, 544, 4240, 3580, 1046, 548, 549, 2598, 552, 553, 3819, 1259, 4062, 554, 2841, 3515, 1433, 1323, 4301, 555, 558, 3738, 1567, 559, 1054, 1270, 3857, 2175, 1257, 1238, 3662, 561, 1058, 563, 4872, 2097, 1061, 4203, 1899, 3981, 1062, 1303, 4116, 570, 1946, 3620, 1514, 4002, 3679, 3964, 3707, 3666, 1348, 4273, 1804, 4497, 571, 1401, 4438, 1895, 1264, 1912, 573, 3939, 4170, 4056, 1271, 1521, 1221, 1451, 3934, 4080, 4352, 1700, 3739, 1419, 1811, 3950, 1690, 3716, 1569, 1437, 1674, 4337, 1285, 4268, 1183, 3881, 3938, 4198, 4416, 4229, 4292, 4193, 4325, 4375, 4432, 4215, 3944, 1409, 4195, 4223, 1540, 4455, 4512, 1723, 3750, 4070, 4104, 4235, 1863, 4346, 1881, 1071, 1814, 4443, 3761, 1346, 1587, 3592, 576, 4469, 1275, 1950, 4129, 1296, 1551, 3733, 4111, 3993, 4108, 3949, 1287, 1713, 1556, 1736, 1995, 1705, 1861, 4120, 4417, 1292, 1785, 1236, 4293, 1827, 3839, 3983, 4395, 1957, 3928, 4295, 3715, 3855, 3609, 4076, 4175, 3927, 1757, 4299, 1773, 1364, 1462, 4007, 3729, 3665, 3722, 1691, 1712, 1624, 1751, 1394, 3924, 3952, 581, 2003, 1559, 1250, 1555, 4114, 1818, 1076, 4448, 1078, 1786, 3354, 1832, 3432, 2891, 4583, 1903, 1080, 4459, 589, 1563, 1084, 591, 592, 594, 595, 1858, 596, 598, 1453, 2559, 2590, 2738, 4478, 4069, 1809, 1327, 4150, 3260, 3481, 3672, 3625, 4439, 3852, 3997, 3847, 4050, 3377, 4424, 4143, 4493, 3492, 1412, 601, 4017, 4477, 1794, 602, 4263, 3072, 2009, 1754, 1088, 3888, 603, 604, 1368, 3827, 4103, 1571, 1438, 2069, 611, 1093, 612, 1094, 3701, 4389, 614, 5000, 615, 1332, 1715, 4369, 1652, 1541, 1921, 1733, 1373, 1870, 619, 4218, 1798, 1702, 1384, 3700, 1788, 1600, 624, 2473, 1423, 2771, 1339, 3875, 3070, 2015, 2557, 2795, 2906, 2103, 3299, 3118, 4838, 4965, 4605, 4576, 1103, 1614, 1947, 631, 4479, 3766, 1107, 635, 1813, 636, 2431, 637, 1109, 4577, 639, 3706, 1110, 640, 1111, 4581, 4644, 4734, 642, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 1269, 3095, 1286, 3624, 4481, 3511, 2735, 4381, 3295, 1585, 1568, 1766, 3221, 2643, 2739, 4629, 3920, 4603, 2307, 647, 1801, 4279, 3762, 1637, 1509, 4590, 648, 1117, 4800, 4724, 4674, 1119, 2965, 2695, 3293, 4722, 1121, 4784, 653, 1122, 3264, 655, 656, 657, 4035, 2910, 3391, 2808, 3099, 2913, 3676, 2852, 3284, 2731, 3537, 3994, 1319, 4467, 1623, 660, 4714, 4812, 1123, 664, 2138, 1126, 1128, 1129, 1130, 670, 1527, 671, 1772, 1133, 2196, 1136, 1137, 5005, 1933, 1731, 1793, 3342, 1140, 1141, 4525, 687, 3822, 1601, 1148, 693, 3905, 694, 1414, 1149, 4756, 4859, 2258, 697, 1795, 1372, 1695, 1866, 1151, 4115, 1719, 2194, 1920, 4999, 1544, 703, 4031, 4305, 1636, 1393, 3810, 4452, 3677, 4227, 4402, 4157, 4197, 1337, 1253, 1532, 4028, 3989, 4715, 704, 1493, 1902, 1152, 4824, 4182, 707, 708, 709, 4732, 4476, 1375, 713, 714, 715, 1841, 4234, 1154, 1314, 4096, 4895, 718, 719, 720, 2063, 1311, 721, 4903, 4885, 4217, 1627, 2116, 1158, 724, 5013, 4894, 1953, 1201, 3787, 729, 2485, 3747, 730, 1846, 3815, 1964, 1822, 1262, 4480, 1537, 1291, 1318, 4976, 4294, 1582, 4587, 3439, 2833, 4287, 4509, 4505, 4267, 3697, 3272, 4112, 2967, 3651, 1805, 3705, 1195, 3859, 1164, 1165, 2351, 4835, 4571, 2423, 1745, 1973, 1168, 3807, 4307, 1169, 1454, or 1781.

A VP capsid polypeptide may have a 581 to 589 region having a sequence of SEQ ID NO: 5014, wherein X³ of SEQ ID NO: 5014 is a basic amino acid residue and X⁶ of SEQ ID NO: 5014 is a basic amino acid residue. In some embodiments, the 581 to 589 region has a sequence of any one of SEQ ID NOs: 4177, 1289, 740, 3477, 1548, 3051, 2982, 2877, 3036, 3426, 3073, 3169, 747, 3362, 2784, 21, 3192, 1638, 748, 1703, 1836, 3660, 29, 750, 1640, 33, 35, 36, 1205, 3795, 753, 4288, 4219, 2770, 2962, 4241, 4107, 2718, 3317, 4391, 3263, 2553, 3947, 2710, 2608, 3467, 2796, 2961, 2529, 3320, 2674, 2635, 3446, 3053, 3250, 3688, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 3327, 3178, 3006, 3252, 2991, 3470, 2888, 2572, 3418, 2642, 2519, 3363, 40, 2633, 45, 756, 48, 1952, 3122, 2551, 3718, 3484, 3577, 759, 3960, 1378, 2994, 3080, 2589, 3681, 760, 762, 3925, 3654, 60, 4086, 1740, 1660, 62, 1966, 63, 768, 66, 67, 68, 3816, 2599, 2706, 74, 3865, 1549, 1361, 2453, 3882, 3713, 1942, 1312, 4501, 76, 1560, 4289, 3757, 3593, 3908, 4397, 4415, 3723, 3754, 4360, 3945, 2854, 3523, 3669, 4065, 1294, 1484, 3891, 4456, 4040, 4496, 1982, 2001, 1478, 1189, 1490, 1885, 1926, 89, 1180, 92, 786, 94, 95, 1831, 1867, 4458, 97, 1970, 1936, 1590, 99, 789, 790, 1298, 101, 1688, 791, 792, 2007, 103, 104, 105, 108, 1579, 110, 1906, 1444, 1344, 116, 802, 117, 118, 121, 803, 1463, 124, 807, 133, 1978, 812, 135, 136, 137, 138, 140, 1326, 141, 142, 143, 1634, 1302, 817, 818, 147, 148, 149, 151, 154, 155, 819, 820, 157, 165, 822, 168, 169, 170, 823, 1651, 173, 1483, 174, 825, 176, 180, 827, 181, 828, 184, 185, 186, 188, 189, 830, 192, 193, 194, 196, 1391, 197, 200, 201, 202, 203, 205, 832, 206, 834, 835, 836, 1334, 3901, 3312, 2649, 1450, 4386, 3028, 1446, 215, 1522, 3490, 217, 219, 1612, 220, 221, 222, 839, 223, 840, 224, 230, 231, 842, 232, 233, 235, 851, 853, 1536, 240, 242, 243, 244, 245, 246, 247, 250, 251, 254, 255, 857, 256, 258, 259, 859, 260, 861, 862, 2326, 264, 1241, 266, 1477, 867, 869, 870, 271, 871, 872, 272, 1252, 1591, 275, 1980, 276, 876, 877, 277, 278, 1892, 1539, 3601, 3877, 1930, 1460, 282, 1799, 1626, 1374, 1620, 1290, 1354, 1956, 889, 292, 4324, 4138, 891, 3903, 297, 3829, 3809, 4271, 3844, 1436, 1617, 1961, 304, 1589, 4370, 3646, 4059, 1431, 1331, 312, 1779, 313, 896, 1558, 1552, 322, 1958, 324, 901, 1491, 329, 330, 4484, 1756, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 4248, 1557, 1602, 4188, 1330, 4349, 3337, 3533, 2629, 3038, 2539, 3567, 3291, 3348, 3115, 3359, 2993, 3079, 3506, 2527, 2963, 2938, 2516, 3113, 2639, 3143, 3044, 2747, 3154, 3280, 3039, 2743, 2902, 3056, 2554, 2522, 3103, 3210, 3434, 2656, 4251, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 2869, 2610, 3298, 2775, 3108, 2603, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3629, 3578, 3885, 2817, 3248, 2954, 3334, 3571, 3345, 3551, 3384, 3422, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3120, 3286, 2843, 3465, 3946, 4222, 2682, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 4269, 3487, 3489, 3019, 1266, 3435, 3482, 3020, 2748, 2646, 2787, 2760, 2563, 3085, 3133, 3158, 3170, 2703, 3494, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 3689, 2968, 1457, 3450, 2577, 2567, 2532, 3415, 2934, 2984, 2568, 4147, 3002, 2986, 2979, 2542, 1762, 4472, 1529, 4057, 1976, 4507, 1583, 1711, 2013, 1594, 4440, 344, 921, 4498, 3728, 3637, 3570, 3429, 4091, 2870, 2995, 4338, 3205, 2880, 3261, 3968, 4291, 4204, 4071, 4034, 1742, 2820, 3912, 3287, 4145, 354, 1782, 927, 3610, 4224, 1918, 1959, 3736, 1770, 931, 4155, 364, 1279, 365, 1543, 367, 4049, 1633, 1722, 369, 1495, 1566, 1211, 1748, 4142, 1434, 1358, 1191, 3732, 4110, 4039, 4411, 4506, 4156, 4036, 4030, 4016, 3899, 1247, 1855, 3919, 1244, 4140, 3930, 4117, 4189, 4001, 3643, 3619, 1240, 1575, 3871, 4135, 4408, 3604, 1274, 3741, 1938, 3709, 3622, 3834, 1983, 1743, 3979, 4461, 4113, 3817, 4232, 4214, 1219, 1486, 1512, 1516, 4047, 4082, 4094, 1574, 1248, 4078, 1872, 1426, 3785, 3687, 4318, 4243, 3866, 3704, 4172, 1513, 1856, 3324, 2769, 2678, 1561, 3296, 377, 1395, 4093, 4453, 3778, 4210, 4009, 4264, 4339, 1738, 4194, 4474, 4013, 4266, 1771, 3860, 3832, 1349, 1381, 3663, 1185, 3932, 3858, 1198, 4192, 3611, 1919, 3790, 3712, 4095, 1887, 4468, 1720, 4247, 2002, 1657, 1336, 3894, 1554, 3576, 4314, 3695, 4362, 1815, 3656, 3638, 4454, 1443, 1661, 1359, 3650, 1648, 3831, 4435, 4451, 1993, 3849, 1718, 4068, 1835, 1425, 1428, 4380, 1658, 1452, 4024, 4072, 3791, 4491, 4323, 4014, 1304, 1511, 1981, 1676, 4422, 3748, 4119, 1297, 1299, 4450, 3990, 4029, 3711, 4344, 1578, 4384, 3892, 3674, 3595, 4405, 3973, 4109, 4159, 3744, 4387, 1960, 4275, 4492, 4365, 4118, 3655, 3886, 4278, 3751, 3678, 4353, 3175, 2970, 1228, 3534, 3437, 3406, 3641, 3639, 4470, 3941, 4348, 3587, 3818, 3814, 4350, 3821, 3929, 3824, 4465, 4494, 1398, 3828, 1284, 3773, 4209, 3014, 3884, 2783, 4038, 3545, 2916, 3112, 2834, 3880, 4399, 4048, 3752, 4207, 1235, 3943, 4355, 4087, 4100, 4163, 4486, 4178, 1643, 4152, 3634, 4445, 4303, 4284, 1213, 4382, 1399, 3714, 4487, 3615, 4018, 4420, 4121, 3724, 4282, 4310, 4488, 4127, 3670, 4052, 4124, 4508, 4500, 3652, 3972, 1309, 1528, 3717, 1217, 2740, 3125, 3546, 3842, 4404, 3690, 3962, 4329, 4410, 3703, 4136, 3589, 3907, 4153, 2707, 4180, 3923, 4444, 4361, 4394, 4302, 3758, 4510, 3742, 1710, 1335, 1997, 1701, 4280, 4006, 1984, 3833, 1593, 2561, 4442, 3900, 4298, 1761, 4388, 1597, 4317, 4249, 1341, 1680, 4441, 3628, 3725, 1945, 937, 4206, 1588, 1439, 3794, 4447, 3776, 3658, 4064, 4011, 4272, 4503, 1367, 3594, 3680, 1300, 1750, 3913, 3774, 1877, 1987, 3998, 1380, 4185, 3686, 3696, 4463, 3853, 4244, 4083, 1909, 3897, 1546, 3613, 3273, 3452, 3525, 1229, 3835, 1234, 3970, 3856, 3586, 1175, 4321, 2778, 4412, 4407, 1810, 3823, 1427, 1507, 1878, 4333, 3146, 4281, 4211, 1179, 3843, 1677, 1524, 1525, 1974, 1239, 4167, 3740, 1342, 4125, 1580, 4060, 1576, 1212, 3921, 1174, 4061, 4021, 3693, 3734, 1382, 3684, 3806, 3635, 3976, 3840, 4286, 3836, 4161, 3659, 3743, 3605, 4392, 4328, 1316, 4173, 4265, 1246, 1629, 1829, 1837, 1914, 1839, 3984, 3910, 4160, 4184, 4132, 4250, 1562, 1847, 1792, 3769, 1534, 4003, 1689, 1599, 1265, 1655, 3874, 4261, 3626, 3579, 1644, 3936, 4054, 4260, 4297, 4357, 4022, 4046, 1222, 1996, 3788, 3953, 4226, 4277, 4101, 3797, 4019, 3771, 3632, 1402, 4137, 3772, 3661, 4186, 4174, 1170, 3850, 1315, 1611, 4033, 3623, 3596, 1461, 1848, 1173, 1928, 1765, 3719, 3584, 3591, 4165, 4191, 3980, 4058, 4200, 4336, 3958, 1260, 4051, 3798, 4032, 1774, 4322, 3991, 4106, 3978, 3965, 4464, 3813, 4154, 1365, 3846, 4345, 4379, 3603, 1693, 3937, 3801, 1787, 1214, 4483, 4201, 1898, 1220, 4245, 1347, 3867, 1900, 3640, 3916, 4089, 3745, 3975, 4256, 4308, 4000, 3868, 4403, 3883, 4326, 4393, 3895, 4237, 3955, 4005, 4092, 4372, 4283, 3805, 4151, 3982, 3597, 3872, 3987, 4490, 3876, 3668, 4300, 1679, 1242, 3598, 3583, 3647, 4304, 3779, 1907, 4042, 3648, 3996, 3800, 4367, 4099, 1807, 3887, 4428, 1625, 1390, 1884, 1682, 4364, 4306, 3869, 1278, 942, 1505, 1542, 1709, 3600, 3914, 3948, 1865, 1716, 3961, 1506, 4221, 1376, 1683, 4276, 1721, 1632, 3607, 1455, 1913, 1497, 1317, 1951, 1778, 3768, 4225, 1668, 390, 4406, 4341, 1480, 1714, 1422, 397, 3618, 4433, 1177, 3898, 398, 950, 400, 1965, 4471, 403, 1482, 3602, 4434, 3721, 1592, 1519, 1352, 4409, 1977, 1631, 1684, 409, 3889, 414, 415, 961, 1905, 416, 963, 3675, 1833, 3974, 965, 970, 1897, 4133, 3830, 972, 424, 427, 1708, 4141, 3749, 1619, 975, 4044, 3590, 1944, 4126, 1923, 1706, 4400, 1606, 2604, 2895, 436, 3448, 1692, 3862, 4055, 4358, 985, 1664, 4045, 3854, 3682, 1972, 2688, 4366, 3425, 3231, 3649, 2759, 2724, 3755, 1216, 3599, 3775, 2754, 2606, 3471, 3234, 2565, 2803, 2562, 2684, 3411, 3405, 2690, 1255, 3694, 3535, 1306, 4084, 3282, 1780, 990, 991, 992, 1728, 994, 1696, 998, 3116, 1869, 456, 1386, 3879, 2698, 1001, 461, 1447, 464, 4425, 1231, 1468, 476, 1273, 1744, 1488, 1659, 1604, 481, 4313, 1499, 1016, 4073, 1777, 4274, 487, 1775, 4205, 1526, 1697, 2000, 1882, 1817, 1880, 490, 1018, 3917, 3616, 4311, 1800, 4473, 4085, 4401, 3783, 3765, 491, 3699, 3792, 4290, 4090, 2012, 1803, 3642, 1322, 502, 1333, 1890, 3608, 1732, 511, 2397, 1864, 1816, 1531, 3636, 1699, 1027, 1366, 4027, 3478, 2959, 3197, 1595, 3413, 518, 1675, 1797, 2315, 1999, 3759, 1215, 4122, 1730, 3966, 3911, 3289, 1821, 530, 1874, 1038, 3767, 1929, 3683, 1338, 1517, 4331, 4332, 4466, 2719, 4123, 4233, 1824, 3129, 3087, 3505, 4158, 4254, 3720, 4351, 4414, 3453, 3254, 1649, 544, 4240, 3580, 1046, 2598, 552, 3819, 1259, 4062, 2841, 3515, 4301, 3738, 1567, 1270, 3857, 1257, 1238, 3662, 561, 1058, 1061, 4203, 1899, 3981, 1062, 1303, 4116, 1946, 3620, 1514, 4002, 3679, 3964, 3707, 3666, 1348, 4273, 1804, 4497, 1401, 4438, 1895, 1264, 1912, 3939, 4170, 4056, 1271, 1521, 1221, 1451, 3934, 4080, 4352, 1700, 3739, 1419, 1811, 3950, 1690, 3716, 1437, 1674, 4337, 1285, 4268, 1183, 3881, 3938, 4198, 4416, 4229, 4292, 4193, 4325, 4375, 4432, 4215, 3944, 1409, 4195, 4223, 1540, 1723, 3750, 4070, 4104, 4235, 1863, 4346, 1881, 1814, 4443, 3761, 1346, 1587, 3592, 576, 4469, 1275, 1950, 4129, 1296, 1551, 3733, 4111, 3993, 4108, 3949, 1287, 1713, 1736, 1995, 1705, 1861, 4120, 4417, 1785, 1236, 4293, 3839, 3983, 4395, 1957, 3928, 4295, 3715, 3855, 3609, 4076, 4175, 3927, 1757, 4299, 1773, 1364, 1462, 4007, 3729, 3665, 3722, 1691, 1624, 1751, 1394, 3924, 3952, 581, 2003, 1559, 1250, 1555, 4114, 1818, 1076, 4448, 1078, 1786, 3354, 1832, 3432, 2891, 1903, 4459, 1563, 591, 594, 598, 1453, 2559, 2590, 2738, 4478, 4069, 1809, 1327, 4150, 3260, 3481, 3672, 3625, 4439, 3852, 3997, 3847, 4050, 3377, 4424, 4143, 4493, 3492, 601, 4017, 4477, 1794, 4263, 3072, 2009, 1754, 1088, 3888, 1368, 3827, 4103, 1571, 1438, 1093, 612, 1094, 3701, 4389, 1332, 1715, 4369, 1652, 1541, 1921, 1733, 1373, 1870, 619, 4218, 1798, 1702, 1384, 3700, 1788, 1600, 624, 2771, 1339, 3875, 3070, 2557, 2795, 2906, 3118, 1614, 1947, 4479, 3766, 635, 1813, 636, 3706, 1110, 640, 1111, 642, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 3095, 1286, 3624, 4481, 3511, 2735, 4381, 3295, 1585, 1766, 3221, 2643, 2739, 3920, 2307, 647, 1801, 4279, 3762, 1637, 1509, 1119, 2965, 2695, 3293, 1121, 1122, 3264, 655, 4035, 2910, 3391, 2808, 3099, 2913, 3676, 2852, 3284, 2731, 3994, 1319, 4467, 1623, 664, 1128, 1129, 1130, 670, 1527, 671, 1772, 5005, 1933, 1731, 1793, 3342, 3822, 1601, 3905, 694, 1414, 1795, 1372, 1695, 1866, 4115, 1719, 1920, 1544, 703, 4031, 4305, 1636, 1393, 3810, 4452, 3677, 4227, 4402, 4157, 4197, 1337, 1253, 1532, 4028, 3989, 1493, 1902, 4182, 707, 708, 4476, 714, 1841, 4234, 1314, 4096, 4217, 1627, 1953, 1201, 3787, 3747, 730, 1846, 3815, 1964, 1822, 1262, 4480, 1537, 1291, 1318, 4294, 1582, 3439, 2833, 4287, 4509, 4505, 4267, 3697, 3272, 4112, 2967, 3651, 3705, 1195, 3859, 1164, 1165, 1745, 1973, 1168, 3807, 4307, 1454, or 1781.

A VP capsid polypeptide may have a 581 to 589 region having a sequence of SEQ ID NO: 5014, wherein X³ of SEQ ID NO: 5014 is K or R. In some embodiments, the 581 to 589 region has a sequence of any one of SEQ ID NOs: 4177, 1289, 740, 741, 18, 745, 3477, 1548, 3051, 2982, 2877, 3036, 3426, 3073, 3169, 747, 3362, 2784, 21, 3192, 23, 1638, 748, 1703, 1836, 3660, 29, 750, 1640, 33, 34, 35, 36, 37, 38, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 3327, 3178, 3006, 3252, 2991, 3470, 2888, 2572, 3418, 2642, 2519, 3363, 40, 2633, 41, 42, 43, 755, 45, 756, 46, 48, 1952, 758, 49, 3122, 2551, 3718, 3484, 3577, 759, 1230, 3960, 1378, 2994, 3080, 2589, 3681, 760, 53, 3925, 55, 3654, 56, 61, 4086, 1740, 1660, 765, 766, 62, 1966, 63, 64, 768, 771, 65, 66, 67, 68, 3816, 2599, 2706, 74, 3865, 1549, 1361, 3882, 3713, 1942, 1312, 4501, 76, 77, 1560, 4289, 3757, 79, 3593, 4043, 1806, 3908, 4397, 1615, 4415, 3723, 2006, 3754, 1470, 4360, 3945, 2854, 3523, 3669, 82, 4065, 83, 1294, 1484, 3891, 4456, 4040, 4496, 1982, 2001, 1478, 1189, 1490, 1885, 1926, 89, 90, 91, 779, 1180, 92, 785, 786, 94, 95, 1831, 1867, 4458, 97, 1970, 1936, 1590, 100, 789, 790, 1298, 101, 1688, 791, 792, 2007, 103, 794, 795, 104, 105, 106, 1849, 108, 1579, 110, 1906, 113, 1444, 1344, 116, 802, 117, 118, 121, 803, 804, 1463, 805, 122, 124, 125, 127, 807, 132, 809, 810, 133, 1978, 812, 135, 136, 137, 138, 813, 140, 815, 1326, 141, 142, 143, 144, 145, 1634, 1302, 817, 146, 818, 147, 148, 149, 151, 152, 153, 154, 155, 819, 820, 157, 159, 160, 163, 164, 165, 166, 822, 167, 168, 169, 170, 171, 823, 824, 1651, 173, 1483, 174, 175, 825, 176, 177, 826, 827, 181, 828, 182, 829, 184, 185, 186, 187, 188, 189, 830, 191, 192, 193, 194, 195, 196, 1391, 197, 200, 201, 202, 203, 204, 205, 832, 833, 206, 834, 836, 1334, 837, 3901, 3312, 2649, 1301, 211, 212, 1450, 4386, 3028, 213, 1446, 214, 215, 1896, 1522, 3490, 838, 217, 218, 219, 1612, 220, 221, 222, 839, 223, 840, 224, 226, 227, 230, 231, 842, 232, 233, 844, 236, 237, 851, 852, 853, 1536, 240, 854, 242, 243, 244, 245, 246, 247, 249, 250, 251, 252, 253, 254, 255, 857, 256, 257, 258, 259, 858, 859, 260, 261, 860, 861, 862, 262, 264, 1241, 265, 266, 866, 1477, 267, 867, 869, 870, 271, 871, 872, 272, 1252, 273, 1591, 275, 1980, 276, 876, 877, 277, 278, 279, 1892, 1539, 880, 1295, 3601, 3877, 1930, 1420, 1678, 1460, 282, 1799, 283, 1626, 1374, 287, 1620, 1290, 1354, 888, 291, 1956, 889, 292, 4324, 293, 4138, 891, 3903, 297, 3829, 3809, 4271, 3844, 1436, 1515, 1617, 1961, 304, 306, 1589, 4370, 308, 309, 894, 3646, 4059, 1431, 1331, 312, 895, 1779, 313, 314, 315, 316, 896, 1558, 1552, 898, 321, 322, 899, 1958, 901, 325, 902, 326, 1491, 329, 330, 905, 4484, 1756, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 1888, 4248, 1557, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 3435, 3482, 3020, 2748, 2646, 2787, 2760, 2563, 3085, 3133, 3158, 3170, 2703, 3494, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 2979, 2542, 1762, 4472, 1472, 1529, 4057, 1976, 4507, 1583, 338, 911, 1711, 340, 912, 2013, 341, 4440, 917, 344, 918, 919, 921, 922, 347, 351, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 4145, 354, 925, 1782, 926, 927, 3610, 357, 359, 4224, 1918, 1959, 3736, 1770, 931, 4155, 364, 1279, 365, 1543, 367, 4049, 1633, 1722, 368, 369, 1495, 1566, 370, 1211, 1873, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 3741, 1938, 3709, 1727, 3893, 1280, 3622, 1784, 3834, 1752, 1983, 1743, 3979, 4461, 4113, 3817, 4232, 1340, 1489, 4214, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 377, 1395, 4453, 3940, 3778, 4374, 4210, 4009, 4264, 4339, 4368, 1738, 378, 1610, 4194, 4474, 4013, 4266, 1771, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 2002, 1657, 1663, 1336, 3894, 1554, 1494, 1405, 3576, 4314, 3695, 1851, 4362, 1815, 3845, 4511, 3656, 3638, 1406, 1596, 4454, 1443, 1661, 1359, 1734, 3650, 1910, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1680, 4441, 3628, 3725, 1945, 4206, 1588, 1439, 3794, 4447, 1838, 3776, 3658, 4064, 1377, 4011, 4272, 4503, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 3613, 3273, 3452, 3525, 1229, 4105, 3835, 383, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 1520, 1767, 1466, 1194, 4137, 1908, 1927, 1758, 3772, 3661, 4186, 4174, 1170, 3850, 1315, 1611, 1530, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 1716, 3961, 1506, 4221, 1376, 1683, 4276, 1445, 1721, 1632, 3607, 1328, 1455, 1913, 1497, 1317, 1951, 1778, 1379, 3768, 4225, 1668, 390, 391, 4406, 4341, 1480, 395, 1714, 1422, 396, 397, 3618, 4433, 1177, 3898, 398, 949, 950, 399, 951, 400, 1965, 4471, 952, 402, 2475, 403, 404, 4139, 1482, 3602, 4434, 3721, 1592, 1519, 1352, 4202, 4409, 1977, 1917, 407, 408, 1631, 1684, 409, 3889, 958, 959, 412, 960, 414, 415, 961, 1905, 416, 963, 417, 3675, 1833, 3974, 420, 421, 965, 966, 969, 970, 422, 1897, 423, 4133, 3830, 972, 424, 973, 427, 1708, 4141, 3749, 431, 1619, 975, 4044, 3590, 1944, 4126, 1923, 1706, 4400, 434, 1606, 979, 2604, 2895, 436, 3448, 1692, 983, 3862, 4055, 4358, 984, 985, 1664, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 3535, 1306, 4084, 3282, 1780, 987, 988, 990, 451, 452, 991, 992, 1728, 994, 995, 454, 1696, 3116, 1869, 456, 1386, 999, 3879, 2698, 1001, 461, 1447, 462, 464, 4425, 1645, 1231, 1468, 472, 473, 476, 1273, 1007, 1744, 1488, 1659, 477, 1604, 481, 4313, 482, 483, 1499, 484, 1016, 4073, 1777, 1458, 4274, 487, 1775, 4205, 1526, 1697, 1017, 2000, 489, 3917, 3616, 4311, 1800, 4473, 4085, 4401, 1310, 3783, 3765, 491, 1948, 3699, 1735, 3792, 4290, 495, 4090, 496, 2012, 1803, 3642, 1322, 498, 1021, 1022, 502, 1333, 503, 1023, 1890, 510, 1026, 3608, 1732, 511, 2397, 1864, 1816, 1531, 3636, 1699, 1027, 1029, 1366, 2112, 1261, 4027, 3478, 3763, 2959, 517, 3197, 1595, 3413, 1448, 518, 1675, 1797, 520, 1999, 3759, 1215, 4122, 1730, 524, 526, 3966, 3911, 529, 3289, 1821, 530, 531, 1874, 1038, 536, 537, 3767, 1929, 1041, 1042, 3683, 542, 1338, 4331, 4332, 4466, 2719, 3803, 4123, 4233, 1824, 3129, 3087, 3505, 4158, 4254, 1449, 3720, 4351, 4414, 1673, 3453, 3254, 1649, 546, 4240, 3580, 1046, 1047, 550, 2598, 551, 552, 3819, 1259, 4062, 2841, 1050, 3515, 4301, 1052, 1053, 3738, 1567, 1270, 3857, 1055, 1257, 1238, 3662, 561, 562, 1058, 4203, 1899, 3981, 1062, 1303, 4116, 567, 1946, 3620, 1514, 4002, 3679, 3964, 3707, 3666, 1348, 4273, 1804, 4497, 1401, 4438, 1895, 1264, 572, 1912, 3939, 4170, 4056, 1271, 1065, 1521, 1221, 1451, 1654, 3934, 4080, 4352, 1700, 3739, 1565, 1419, 1811, 3950, 1690, 1397, 3716, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 1723, 1070, 3750, 3633, 4070, 4104, 4162, 4235, 1863, 4346, 1881, 1814, 1572, 4443, 3761, 1346, 1587, 1072, 3592, 576, 1891, 4469, 1275, 1950, 1963, 4129, 1296, 1551, 3733, 4111, 3993, 4108, 3949, 1287, 1713, 1736, 1995, 1435, 1424, 1830, 1705, 1861, 1197, 1254, 4120, 1545, 4417, 1469, 1785, 1236, 4293, 3839, 3983, 4395, 1957, 3928, 4295, 3715, 3855, 3609, 1990, 4076, 4175, 3927, 1757, 1650, 4299, 1773, 2004, 1267, 1364, 1462, 1879, 4007, 3729, 3665, 3722, 1691, 1624, 1751, 1394, 3924, 3952, 580, 581, 2003, 1343, 1559, 1250, 1555, 4114, 1818, 1075, 1076, 1077, 1704, 4448, 1078, 1079, 1786, 585, 3354, 1832, 3432, 2891, 2505, 1903, 1081, 1082, 4459, 1083, 1563, 590, 591, 1085, 594, 1453, 2559, 2590, 2738, 4478, 4069, 1809, 1327, 4150, 3260, 3481, 3672, 3625, 2008, 4439, 3852, 3997, 1411, 599, 3847, 1268, 4050, 3377, 4424, 1496, 4143, 4493, 3492, 1962, 600, 601, 4017, 4477, 1794, 4263, 3072, 2009, 1754, 1087, 1088, 3888, 1368, 3827, 4103, 606, 1090, 1571, 1091, 1438, 1092, 1093, 612, 1094, 3701, 4389, 613, 1603, 1332, 1715, 4369, 1652, 1096, 617, 1541, 618, 1921, 1733, 1373, 1870, 619, 4218, 1798, 1100, 1702, 1101, 1667, 1384, 623, 3700, 1788, 1600, 624, 626, 2771, 627, 1339, 3875, 628, 629, 3070, 4860, 2557, 2795, 2906, 3118, 1614, 1947, 4479, 3766, 632, 1104, 1105, 633, 1813, 636, 3706, 1110, 640, 1111, 641, 642, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 3095, 1286, 3624, 4481, 1432, 3511, 2735, 4381, 3295, 1585, 1114, 1766, 3221, 2643, 2739, 3920, 644, 1116, 645, 646, 647, 1801, 4279, 3762, 1637, 1509, 1119, 2965, 2369, 2695, 3293, 650, 651, 1120, 652, 1121, 3264, 655, 658, 659, 4035, 2910, 3391, 2808, 3099, 2913, 3676, 2852, 3284, 2731, 1609, 3994, 1319, 4467, 1802, 1227, 1623, 662, 663, 664, 1124, 1125, 1128, 1129, 668, 669, 1130, 670, 1131, 1527, 1132, 671, 1772, 1135, 672, 1933, 1731, 1793, 675, 676, 3342, 1138, 1139, 677, 679, 1142, 1143, 681, 1145, 682, 685, 3822, 689, 690, 691, 1147, 1601, 3905, 694, 695, 1414, 1150, 1795, 1372, 1695, 1866, 4115, 1719, 698, 1920, 699, 700, 1544, 4305, 1636, 1393, 2010, 3810, 4452, 3677, 4227, 1277, 1400, 4402, 4157, 4197, 1337, 1253, 1532, 4028, 3989, 1493, 1854, 1902, 1510, 4182, 706, 707, 708, 4476, 1245, 714, 1841, 4234, 1314, 4096, 717, 1155, 4217, 1156, 723, 1157, 1627, 725, 1159, 1953, 1160, 1201, 727, 3787, 3747, 730, 1846, 3815, 2005, 1964, 1822, 1262, 4480, 1537, 1291, 1318, 4294, 1582, 734, 3439, 2833, 4287, 4509, 4505, 4267, 3697, 3272, 4112, 2967, 3651, 1628, 3705, 1195, 3859, 1164, 1165, 1166, 1745, 1973, 1168, 3807, 4307, 1454, or 1781.

A VP capsid polypeptide may have a 581 to 589 region having a sequence of SEQ ID NO: 5014, wherein X⁶ of SEQ ID NO: 5014 is K or R. In some embodiments, the 581 to 589 region has a sequence of any one of SEQ ID NOs: 738, 4177, 1289, 740, 14, 743, 744, 17, 3477, 1548, 3051, 2982, 2877, 746, 19, 3036, 3426, 3073, 3169, 747, 3362, 2784, 21, 3192, 22, 1638, 748, 24, 749, 27, 1703, 1836, 28, 3660, 29, 750, 1640, 33, 35, 36, 1653, 1205, 3795, 753, 4288, 4219, 2770, 2962, 4241, 4107, 2718, 3317, 4391, 3263, 2553, 3947, 2710, 2608, 3467, 2796, 2961, 2529, 3320, 2674, 2635, 3446, 3053, 3250, 3688, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 1467, 3327, 3178, 3006, 3252, 2991, 3470, 2888, 2572, 3418, 2642, 2519, 3363, 40, 2633, 44, 45, 756, 47, 48, 1952, 3122, 2551, 3718, 3484, 3577, 1308, 3960, 1378, 2994, 3080, 2589, 3681, 51, 760, 762, 3925, 3654, 763, 59, 60, 4086, 1740, 1660, 62, 1966, 63, 2438, 768, 66, 67, 68, 3816, 2599, 2706, 72, 73, 74, 3865, 1549, 1361, 3882, 773, 3713, 1942, 1312, 4501, 76, 1413, 775, 1560, 4289, 3757, 3593, 3908, 4397, 4415, 3723, 3754, 4360, 3945, 2854, 3523, 3669, 1356, 4065, 1294, 1484, 85, 86, 3891, 4456, 4040, 4496, 1982, 2001, 1478, 1189, 1490, 1885, 1926, 89, 780, 1180, 92, 781, 782, 783, 786, 94, 95, 788, 1831, 1867, 96, 4458, 97, 1970, 1936, 1590, 98, 99, 789, 790, 1298, 1688, 791, 792, 2007, 102, 103, 104, 105, 107, 108, 1579, 1906, 111, 798, 112, 799, 800, 1444, 801, 114, 115, 1344, 116, 802, 117, 118, 119, 121, 803, 1463, 1760, 806, 126, 128, 807, 129, 131, 808, 133, 1978, 811, 812, 135, 136, 137, 138, 814, 140, 1326, 141, 142, 143, 2344, 816, 1634, 1302, 817, 818, 147, 148, 149, 150, 151, 154, 155, 819, 820, 156, 157, 158, 161, 162, 165, 822, 168, 169, 170, 823, 172, 1651, 173, 1483, 174, 825, 176, 178, 180, 827, 181, 828, 4911, 184, 186, 188, 189, 830, 192, 193, 194, 196, 1391, 198, 199, 200, 201, 202, 203, 205, 832, 206, 834, 207, 208, 209, 210, 835, 836, 1334, 3901, 3312, 2649, 1450, 4386, 3028, 1446, 1522, 3490, 217, 219, 1612, 220, 221, 222, 839, 223, 840, 224, 225, 228, 229, 230, 231, 842, 232, 233, 234, 238, 847, 239, 850, 851, 853, 1536, 240, 241, 242, 243, 244, 245, 247, 250, 251, 254, 255, 857, 256, 258, 259, 859, 260, 861, 862, 864, 263, 2318, 865, 2326, 264, 1241, 266, 1477, 867, 268, 868, 269, 270, 869, 870, 271, 871, 872, 272, 873, 1252, 1591, 875, 274, 275, 276, 876, 877, 277, 278, 280, 1892, 1539, 881, 3601, 3877, 1460, 282, 1799, 284, 1626, 286, 1374, 1620, 1290, 1354, 887, 1956, 889, 292, 4324, 4138, 891, 296, 3903, 297, 3829, 3809, 4271, 3844, 1436, 300, 1617, 1961, 302, 304, 305, 2147, 1589, 4370, 307, 3646, 4059, 1431, 1331, 312, 1779, 313, 896, 1558, 1552, 318, 319, 322, 1958, 323, 324, 901, 1491, 328, 329, 4484, 907, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3414, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 4248, 1557, 3804, 1602, 4188, 1330, 4349, 3337, 3533, 2629, 3038, 2539, 3567, 3291, 3348, 3115, 3359, 2993, 3079, 3506, 2527, 2963, 2938, 2516, 3113, 2639, 3143, 3044, 2747, 3154, 3280, 3039, 2743, 2902, 3056, 2554, 2522, 3103, 3210, 3434, 2656, 4251, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 2869, 2610, 3298, 2775, 3108, 2603, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 2584, 2712, 3213, 3451, 2634, 3150, 2810, 3303, 2573, 3460, 2615, 3394, 2828, 3329, 4377, 4499, 3555, 2974, 3629, 3578, 3885, 2817, 3248, 2954, 3334, 3571, 3345, 3551, 3384, 3422, 2530, 2973, 4418, 4081, 3951, 1764, 1671, 1843, 3449, 3185, 3399, 2786, 3055, 3472, 3397, 2601, 3564, 3277, 3548, 3179, 3407, 3841, 3347, 3142, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3120, 3286, 2843, 3465, 3946, 2682, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 4269, 3487, 3489, 3019, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1464, 1886, 2666, 3246, 3130, 3560, 3236, 2730, 3572, 3689, 2968, 1457, 3450, 2577, 2567, 2532, 3415, 2934, 2984, 2568, 4147, 3002, 2986, 910, 2979, 2542, 1762, 4472, 1529, 4057, 1976, 4507, 1583, 1711, 2013, 1594, 4440, 344, 920, 921, 4498, 3728, 3637, 3570, 3429, 4091, 2870, 2995, 4338, 3205, 2880, 3261, 3968, 4291, 4204, 4071, 4034, 1742, 2820, 3912, 3287, 1218, 1190, 4145, 354, 1782, 927, 3610, 356, 4224, 1918, 1959, 1840, 3736, 1770, 929, 930, 931, 4155, 360, 361, 362, 364, 1279, 365, 1543, 366, 367, 4049, 1633, 1722, 369, 1495, 1566, 373, 1211, 1726, 1442, 1748, 4142, 1434, 1358, 1191, 3732, 4110, 4039, 4506, 4156, 4036, 4030, 4016, 3899, 1247, 1855, 3919, 1244, 4140, 3930, 4117, 4189, 4001, 3643, 3619, 1240, 1575, 3871, 4135, 4408, 3604, 1274, 4149, 3741, 1938, 3709, 3622, 3834, 1983, 1743, 3979, 4461, 4113, 3817, 4232, 4214, 3902, 1219, 1486, 1512, 1516, 4047, 4082, 4094, 1574, 1248, 4078, 1872, 1426, 3687, 4318, 4243, 3866, 3704, 4172, 1856, 3324, 2769, 2678, 1561, 3296, 377, 1395, 1584, 4093, 4453, 4210, 4009, 4264, 4339, 1738, 4474, 4013, 4266, 1771, 1759, 1922, 3860, 3832, 1349, 1381, 3663, 1185, 3932, 3858, 1198, 4192, 3611, 1919, 3790, 3712, 4095, 1887, 4468, 1720, 4247, 934, 1790, 2002, 1657, 1336, 3894, 3576, 4314, 3695, 1850, 4362, 1815, 3638, 4454, 1661, 4504, 1359, 3650, 1305, 1648, 3831, 4435, 4451, 1993, 3849, 1718, 4068, 1835, 1425, 1428, 4380, 1658, 1452, 4024, 4072, 3791, 4491, 4323, 4014, 1304, 1511, 1981, 1676, 4422, 1283, 3861, 3644, 4259, 1418, 1176, 4216, 1421, 3992, 4423, 4253, 3748, 4119, 1297, 1954, 4450, 3711, 4344, 1578, 4384, 3892, 3674, 3595, 4405, 3973, 4109, 4159, 3744, 4387, 4275, 4492, 4365, 4118, 4278, 3751, 3678, 4353, 3175, 2970, 1228, 3534, 3437, 3406, 3641, 4470, 3941, 4348, 3587, 3818, 4350, 3821, 3929, 3824, 4465, 1398, 3828, 1284, 3773, 4209, 3014, 3884, 2783, 4038, 3545, 2916, 3112, 2834, 3880, 4399, 4048, 3752, 4207, 1235, 3943, 4355, 4087, 4100, 4163, 4486, 4178, 1643, 4152, 4445, 4303, 4284, 1213, 4382, 1399, 3714, 4487, 3615, 4018, 4420, 4121, 3724, 4282, 4310, 4488, 4127, 3670, 4052, 3627, 3777, 1371, 3685, 4258, 3812, 1581, 4124, 4508, 4500, 3652, 3972, 1528, 3717, 2740, 3125, 3546, 3842, 4404, 3690, 3962, 4136, 3589, 3907, 4153, 2707, 4180, 3923, 4444, 4361, 4394, 3758, 4510, 3742, 1710, 1407, 4187, 1193, 1188, 1196, 1724, 1717, 1200, 1997, 1701, 4280, 4006, 1984, 3833, 2561, 4442, 3900, 4298, 1761, 4388, 1597, 4317, 4249, 1341, 1203, 1680, 4441, 3628, 3725, 1945, 937, 4206, 1588, 1439, 3794, 4447, 3776, 3658, 4064, 4011, 4272, 4503, 1324, 3594, 3680, 1300, 1750, 3913, 3774, 1877, 1987, 3998, 3686, 3696, 4463, 3853, 4244, 4083, 1909, 3897, 1546, 3613, 3273, 3452, 3525, 1229, 3835, 1234, 3970, 3856, 3586, 1175, 4321, 2778, 4412, 4407, 3823, 1427, 1507, 1878, 4333, 3146, 4281, 4211, 3843, 1677, 1524, 1525, 1974, 1239, 4167, 3740, 1342, 1471, 4176, 1232, 1989, 4125, 1741, 1580, 4060, 1576, 1212, 3921, 1174, 4061, 4021, 3693, 3734, 1382, 3684, 3806, 3635, 3976, 3840, 4286, 3836, 4161, 3659, 3605, 4392, 4328, 1316, 4173, 4265, 1246, 1629, 1829, 3826, 1992, 1656, 1914, 1839, 3984, 3910, 4160, 4184, 4132, 4250, 1792, 3769, 1534, 4003, 1689, 1599, 1265, 1410, 1823, 1498, 1329, 386, 940, 1655, 3874, 4261, 3626, 3579, 1644, 3936, 4054, 4260, 4297, 4357, 4022, 4046, 1222, 1996, 3788, 3953, 4226, 4277, 4101, 3797, 4019, 3771, 3632, 1402, 4137, 3772, 3661, 4186, 4174, 1170, 3850, 1315, 1611, 1859, 4183, 1403, 4033, 3623, 3596, 1461, 1848, 1173, 1928, 1765, 3719, 3584, 4165, 4191, 3980, 4058, 4200, 4336, 3958, 1260, 4051, 3798, 4032, 1774, 4322, 3991, 4106, 3978, 3965, 4464, 3813, 4154, 1365, 3846, 4345, 4379, 3603, 3937, 3801, 1787, 1214, 4483, 4201, 1845, 1220, 4245, 1347, 3867, 1900, 3640, 3916, 4089, 3745, 3975, 4256, 4308, 4000, 3868, 4403, 3883, 4326, 4393, 3895, 4237, 3955, 4005, 4092, 4372, 4283, 3805, 4151, 3982, 3597, 3872, 3987, 4490, 3876, 3668, 4300, 1679, 1242, 3598, 3583, 3647, 4304, 3779, 1907, 4042, 3648, 3996, 3800, 4367, 4099, 1807, 3887, 4428, 1625, 1390, 1884, 1682, 4364, 4306, 3869, 1278, 942, 1505, 1709, 3600, 3914, 3948, 1865, 1716, 3961, 1506, 4221, 1376, 1683, 4276, 1721, 1632, 3607, 1455, 1913, 1497, 1317, 1951, 1778, 3768, 4225, 1668, 390, 4406, 4341, 1480, 1714, 1422, 397, 3618, 4433, 1177, 3898, 398, 950, 400, 1965, 4471, 953, 401, 403, 955, 1482, 3602, 4434, 3721, 1592, 1825, 1698, 1519, 1352, 4409, 1977, 405, 1631, 1684, 409, 3889, 2117, 411, 413, 414, 415, 961, 1905, 416, 963, 3675, 1833, 3974, 965, 970, 1897, 1272, 4133, 3830, 972, 424, 427, 1708, 4141, 3749, 1619, 975, 4044, 3590, 1944, 4126, 1923, 1706, 432, 976, 978, 4400, 1606, 980, 2604, 2895, 436, 3448, 2188, 438, 1692, 442, 3862, 4055, 4358, 2276, 985, 1664, 443, 4309, 4045, 3854, 3682, 1972, 2688, 4366, 3425, 3231, 3649, 2759, 2724, 3755, 1216, 3599, 3775, 2754, 2606, 3471, 3234, 2565, 2803, 2562, 2684, 3411, 3405, 2690, 1255, 3694, 3985, 3535, 1306, 4084, 3282, 1780, 448, 990, 991, 1485, 992, 1728, 993, 994, 1696, 997, 3116, 1869, 456, 1386, 1000, 3879, 2698, 460, 1001, 461, 1447, 1002, 1003, 463, 464, 4425, 465, 1004, 466, 469, 1231, 1468, 470, 1005, 476, 1273, 1744, 1488, 1659, 1604, 479, 1013, 1014, 481, 4313, 1499, 1016, 4073, 1777, 485, 4274, 487, 1775, 4205, 1526, 1697, 2000, 488, 1894, 1882, 1817, 1880, 490, 1018, 3917, 3616, 4311, 4473, 4085, 4401, 3783, 3765, 1538, 3870, 491, 3699, 3792, 4290, 494, 2421, 4090, 2012, 1803, 3642, 1322, 1020, 502, 1333, 504, 1890, 507, 3608, 1732, 1864, 1816, 1531, 513, 3636, 514, 1699, 1027, 1366, 4950, 4027, 3478, 2959, 1293, 3197, 1595, 3413, 518, 1032, 1033, 1675, 1797, 2315, 521, 1999, 3759, 1215, 4122, 1730, 3966, 1035, 4973, 528, 3911, 3289, 1821, 530, 532, 1874, 533, 1038, 538, 539, 3767, 1929, 3683, 1338, 1616, 1901, 1796, 1517, 4331, 4332, 4466, 2719, 4123, 4233, 1824, 3129, 3087, 3505, 4158, 4254, 3720, 4351, 4414, 3453, 3254, 1649, 544, 4240, 3580, 1046, 548, 549, 2598, 552, 553, 3819, 1259, 4062, 554, 2841, 3515, 1433, 1323, 4301, 555, 558, 3738, 1567, 559, 1054, 1270, 3857, 2175, 1257, 1238, 3662, 561, 1058, 563, 1061, 4203, 1899, 3981, 1062, 1303, 4116, 1946, 3620, 1514, 4002, 3679, 3964, 3707, 3666, 1348, 4273, 1804, 4497, 1401, 4438, 1895, 1264, 1912, 573, 3939, 4170, 4056, 1271, 1521, 1221, 1451, 3934, 4080, 4352, 1700, 3739, 1419, 1811, 3950, 1690, 3716, 1569, 1437, 1674, 4337, 1285, 4268, 1183, 3881, 3938, 4198, 4416, 4229, 4292, 4193, 4325, 4375, 4432, 4215, 3944, 4195, 4223, 1540, 4455, 4512, 1723, 3750, 4070, 4104, 4235, 1863, 4346, 1881, 1814, 4443, 3761, 1587, 3592, 576, 4469, 1275, 1950, 4129, 1296, 1551, 3733, 4111, 3993, 4108, 1287, 1713, 1556, 1736, 1995, 1705, 1861, 4120, 4417, 1785, 1236, 4293, 1827, 3839, 3983, 4395, 1957, 3928, 4295, 3715, 3855, 3609, 4076, 4175, 3927, 1757, 4299, 1773, 1364, 1462, 4007, 3729, 3665, 3722, 1691, 1712, 1624, 1751, 1394, 3924, 3952, 581, 2003, 1559, 1250, 1555, 4114, 1818, 1076, 4448, 1078, 1786, 3354, 1832, 3432, 2891, 1903, 4459, 589, 1563, 1084, 591, 592, 594, 595, 1858, 596, 1453, 2559, 2590, 2738, 4478, 4069, 1809, 1327, 4150, 3260, 3481, 3672, 3625, 4439, 3852, 3997, 3847, 4050, 3377, 4424, 4143, 4493, 3492, 1412, 4017, 4477, 1794, 602, 4263, 3072, 2009, 1754, 3888, 603, 604, 1368, 3827, 4103, 1571, 1438, 611, 1093, 612, 1094, 3701, 4389, 614, 615, 1332, 1715, 4369, 1652, 1541, 1921, 1733, 1373, 1870, 619, 4218, 1798, 1702, 1384, 3700, 1788, 1600, 624, 2473, 1423, 2771, 1339, 3875, 3070, 2015, 2557, 2795, 2906, 2103, 3299, 3118, 4576, 1103, 1614, 1947, 631, 4479, 3766, 1107, 635, 1813, 636, 2431, 637, 1109, 639, 3706, 1110, 640, 642, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 1269, 3095, 1286, 3624, 4481, 3511, 2735, 4381, 3295, 1568, 1766, 3221, 2643, 2739, 3920, 647, 1801, 4279, 3762, 1637, 1509, 648, 1117, 1119, 2965, 2695, 3293, 4722, 1121, 653, 1122, 3264, 655, 656, 657, 4035, 2910, 3391, 2808, 3099, 2913, 3676, 2852, 3284, 2731, 3537, 3994, 1319, 4467, 1623, 660, 1123, 664, 1126, 1128, 1129, 1130, 670, 1527, 671, 1772, 1137, 5005, 1933, 1731, 1793, 3342, 1140, 1141, 687, 3822, 1601, 1148, 693, 3905, 694, 1414, 1149, 697, 1795, 1372, 1695, 1866, 1151, 4115, 1719, 1920, 4999, 1544, 703, 4031, 4305, 1636, 3810, 4452, 3677, 4227, 4402, 4157, 4197, 1337, 1253, 4028, 3989, 704, 1493, 1902, 1152, 4182, 707, 708, 709, 4476, 1375, 713, 714, 1841, 4234, 1154, 1314, 4096, 718, 719, 720, 2063, 1311, 721, 4217, 1627, 1158, 1953, 1201, 3787, 729, 3747, 730, 1846, 3815, 1964, 1822, 1262, 4480, 1537, 1291, 1318, 4294, 1582, 3439, 2833, 4287, 4509, 4505, 4267, 3697, 3272, 4112, 2967, 3651, 1805, 3705, 1195, 3859, 1164, 1165, 2351, 2423, 1745, 1973, 3807, 4307, 1169, 1454, or 1781.

A VP capsid polypeptide may have a 581 to 589 region having a sequence of SEQ ID NO: 5014, wherein X³ of SEQ ID NO: 5014 is not D. In some embodiments, the 581 to 589 region has a sequence of any one of SEQ ID NOs: 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 2469, 4889, 21, 3192, 4942, 22, 23, 1638, 748, 24, 25, 749, 26, 27, 1703, 1836, 28, 2038, 2301, 3660, 29, 750, 30, 31, 32, 2095, 1640, 33, 34, 35, 36, 37, 4742, 2450, 38, 4925, 39, 751, 752, 2144, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2080, 2072, 2034, 50, 1308, 1230, 3960, 1378, 2994, 3080, 2589, 3681, 51, 2167, 52, 760, 53, 2389, 4755, 761, 4591, 762, 4659, 54, 2146, 3925, 55, 3654, 2087, 2346, 4766, 4682, 4984, 4744, 56, 763, 764, 4626, 4702, 57, 58, 59, 60, 61, 4086, 1740, 1660, 765, 766, 62, 1966, 2032, 2499, 2332, 767, 63, 2096, 2181, 4913, 2438, 2041, 64, 768, 769, 770, 2384, 2402, 771, 65, 66, 2111, 67, 772, 2458, 2197, 68, 3816, 69, 2599, 2706, 2497, 70, 2399, 71, 4769, 2366, 72, 73, 74, 3865, 1549, 1361, 2453, 3882, 773, 3713, 1942, 2140, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2379, 81, 82, 4065, 83, 84, 2030, 1294, 2413, 2064, 1484, 2299, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 93, 2356, 784, 785, 786, 94, 95, 788, 1831, 1867, 96, 4458, 97, 1970, 4656, 2173, 1936, 2349, 1590, 98, 99, 100, 789, 790, 1298, 101, 1688, 791, 792, 2007, 4556, 2070, 102, 793, 4632, 103, 794, 795, 104, 105, 106, 2229, 796, 4980, 4691, 107, 2440, 1849, 108, 2014, 797, 4736, 2509, 2018, 2227, 4787, 1579, 110, 1906, 111, 2428, 798, 2075, 112, 2411, 113, 2359, 799, 4848, 800, 2461, 1444, 2313, 2350, 4579, 4821, 801, 114, 2329, 4864, 4834, 115, 1344, 116, 802, 117, 118, 119, 4929, 4677, 120, 4791, 2158, 121, 803, 804, 1463, 805, 122, 2139, 2290, 1760, 2465, 123, 2424, 2058, 2230, 124, 4717, 806, 125, 126, 2370, 127, 4521, 128, 4971, 4994, 807, 2085, 4533, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 4544, 5009, 144, 145, 816, 4654, 2125, 4663, 4810, 4816, 1634, 1302, 817, 146, 818, 147, 148, 149, 4546, 150, 151, 152, 153, 154, 155, 819, 820, 156, 2149, 157, 2295, 821, 158, 159, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 2170, 4809, 4520, 180, 826, 827, 181, 828, 4695, 2130, 4911, 182, 4582, 4873, 2189, 183, 4641, 829, 184, 185, 2244, 4647, 186, 187, 188, 189, 190, 2114, 830, 191, 192, 193, 194, 2071, 4615, 2361, 195, 4852, 2187, 4914, 4947, 196, 1391, 197, 4534, 4726, 4788, 198, 199, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2090, 2241, 217, 218, 4653, 219, 1612, 220, 221, 222, 839, 223, 4974, 4790, 4915, 2464, 4567, 4774, 4964, 2360, 840, 224, 4928, 4741, 225, 2088, 4751, 226, 227, 4890, 4519, 4883, 228, 4786, 229, 230, 231, 2393, 2448, 841, 842, 232, 233, 4782, 234, 2269, 843, 235, 844, 236, 237, 4930, 845, 846, 238, 847, 239, 848, 849, 2150, 850, 851, 852, 853, 1536, 240, 854, 241, 242, 243, 244, 245, 246, 247, 248, 4696, 855, 4634, 856, 249, 250, 251, 252, 253, 254, 255, 857, 2115, 4880, 2218, 256, 257, 258, 259, 858, 859, 260, 261, 860, 861, 862, 262, 4966, 863, 864, 263, 4749, 4970, 2296, 2318, 4535, 865, 2326, 264, 1241, 265, 266, 866, 1477, 267, 867, 4977, 268, 868, 269, 4668, 5002, 270, 869, 870, 271, 871, 872, 272, 4822, 4709, 873, 4655, 4972, 2392, 2317, 874, 1252, 273, 1591, 4975, 875, 274, 2025, 275, 1980, 276, 876, 877, 2066, 277, 278, 279, 4670, 280, 2323, 2288, 2334, 4939, 4689, 281, 878, 2416, 1892, 879, 1539, 2214, 2215, 880, 2327, 881, 882, 4601, 883, 1295, 3601, 3877, 1930, 1420, 1678, 1460, 2306, 884, 282, 1799, 283, 284, 4988, 4871, 4553, 4772, 4817, 2260, 1626, 2082, 885, 286, 1374, 886, 2119, 2504, 2468, 287, 1620, 1290, 2425, 2482, 4564, 4671, 2282, 288, 2354, 1354, 2033, 289, 887, 290, 888, 291, 1956, 2362, 2467, 889, 2357, 4547, 4765, 292, 4794, 2391, 4324, 293, 890, 294, 295, 4138, 891, 2297, 2254, 296, 2224, 3903, 297, 892, 3829, 298, 299, 3809, 4271, 3844, 1436, 300, 1515, 1617, 301, 1961, 4649, 5010, 302, 2060, 303, 304, 305, 2452, 2147, 4574, 4538, 306, 2293, 1589, 893, 4946, 4370, 4841, 2342, 307, 308, 309, 894, 2246, 2155, 3646, 4059, 1431, 1331, 310, 311, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 904, 1635, 4820, 905, 2506, 331, 4706, 4484, 1756, 906, 907, 4661, 2179, 4584, 4609, 332, 4907, 4635, 4529, 4683, 4523, 4561, 333, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4319, 3906, 1533, 1755, 1888, 4248, 1557, 2153, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 2363, 908, 1935, 4711, 4728, 2460, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 4943, 3002, 2986, 910, 2979, 2542, 2338, 2331, 2492, 1762, 4472, 1472, 1529, 2319, 2131, 4057, 4937, 2048, 4887, 1976, 4507, 1583, 338, 2078, 2372, 2102, 339, 911, 1711, 340, 912, 2013, 913, 2277, 914, 2403, 341, 915, 4672, 4902, 916, 342, 1594, 4440, 917, 343, 344, 4559, 918, 2439, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 371, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2345, 4471, 952, 953, 401, 402, 2475, 403, 404, 4758, 954, 955, 2437, 4139, 1482, 3602, 4434, 3721, 1592, 1825, 1698, 1519, 1352, 4202, 4409, 1977, 1917, 405, 406, 407, 408, 956, 957, 2267, 1631, 1684, 409, 2054, 3889, 958, 410, 2117, 411, 959, 412, 960, 2443, 4712, 4619, 2285, 413, 414, 415, 961, 2352, 2417, 962, 1905, 416, 963, 417, 418, 2382, 3675, 1833, 3974, 419, 964, 420, 421, 965, 966, 967, 4919, 968, 2365, 969, 970, 422, 1897, 2056, 2309, 1272, 4754, 2207, 971, 423, 4133, 3830, 4865, 972, 424, 973, 425, 2068, 4955, 426, 974, 2109, 2380, 427, 1708, 4676, 428, 4141, 429, 430, 3749, 431, 1619, 975, 4044, 3590, 1944, 4126, 1923, 1706, 432, 976, 977, 978, 4867, 4400, 433, 434, 1606, 2143, 979, 435, 980, 2604, 2895, 981, 436, 3448, 4959, 4563, 2188, 437, 438, 2055, 1692, 982, 2388, 2243, 983, 2035, 2120, 4940, 439, 440, 441, 2474, 4900, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 2242, 453, 2286, 992, 1728, 993, 4703, 994, 995, 454, 1696, 996, 2039, 455, 997, 4874, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 466, 2328, 467, 468, 469, 1231, 1468, 470, 471, 2496, 472, 473, 1005, 474, 475, 2108, 4725, 1006, 476, 1273, 1007, 1744, 1488, 1659, 477, 478, 4598, 1604, 4753, 2493, 479, 1008, 1009, 480, 1010, 1011, 1012, 1013, 1014, 481, 4313, 482, 483, 1499, 484, 1015, 4761, 2091, 2386, 2513, 1016, 4073, 1777, 2172, 485, 2205, 4592, 4545, 4720, 486, 1458, 4274, 2122, 4905, 4796, 2152, 4539, 4528, 4904, 4842, 4986, 4639, 4846, 4623, 4650, 4595, 2195, 4681, 487, 2182, 4710, 1775, 2083, 4205, 1526, 4586, 1697, 1017, 2000, 4692, 4795, 2480, 2017, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 5001, 2136, 493, 2211, 4532, 494, 2421, 495, 4090, 2378, 2104, 2210, 2247, 2478, 2047, 496, 4620, 4978, 4614, 1019, 2012, 1803, 3642, 497, 1322, 4752, 1020, 498, 1021, 2235, 2302, 2488, 4665, 4699, 4608, 4781, 499, 2049, 2404, 4685, 500, 501, 1022, 502, 1333, 503, 1023, 4686, 4804, 504, 4727, 505, 506, 4938, 4805, 2065, 1890, 4891, 1024, 507, 508, 509, 2110, 1025, 510, 1026, 3608, 1732, 511, 2397, 1864, 1816, 1531, 512, 513, 2451, 3636, 5012, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 2093, 4746, 2489, 4922, 2145, 1030, 4832, 4950, 1261, 4027, 3478, 3763, 2959, 1293, 2462, 517, 3197, 1595, 3413, 2067, 1031, 2228, 1448, 518, 4698, 4616, 2057, 2305, 2133, 2061, 4526, 4617, 1032, 1033, 2429, 2311, 1675, 1797, 519, 4578, 2271, 4693, 4604, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 535, 1038, 536, 1039, 537, 538, 539, 1040, 3767, 1929, 1041, 1042, 3683, 2100, 4853, 2300, 2165, 540, 2105, 541, 1043, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1649, 543, 544, 545, 546, 1045, 547, 2225, 4240, 3580, 4680, 1046, 548, 549, 1047, 550, 2598, 551, 552, 553, 3819, 1259, 4062, 1048, 554, 2841, 1049, 1050, 3515, 2219, 1433, 1323, 4301, 555, 556, 1051, 557, 558, 1052, 1053, 3738, 1567, 559, 1054, 1270, 3857, 1055, 2151, 560, 2175, 2089, 1056, 4514, 2198, 2193, 1257, 1238, 3662, 561, 2186, 1057, 4637, 562, 1058, 563, 4872, 2097, 2436, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 575, 4443, 3761, 1346, 1587, 1072, 3592, 576, 1891, 4469, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2185, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 2304, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2113, 590, 591, 1085, 2256, 592, 2308, 593, 594, 2274, 595, 4931, 2407, 1858, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 2321, 1086, 601, 4017, 4477, 1794, 2483, 4776, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 2320, 2069, 610, 611, 1093, 612, 1094, 3701, 4389, 613, 4897, 1603, 614, 1095, 2154, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4855, 4847, 2398, 2239, 1101, 1667, 1384, 623, 4745, 3700, 1788, 1600, 624, 2098, 625, 4550, 4721, 4960, 4878, 2473, 4792, 4675, 1102, 1423, 626, 2771, 627, 1339, 3875, 628, 629, 3070, 4565, 2118, 2015, 4818, 4860, 2557, 2795, 2906, 2103, 4869, 2029, 3299, 3118, 4932, 4838, 4901, 4908, 4996, 4779, 2377, 4657, 4646, 4875, 4627, 4515, 4876, 4965, 4605, 4576, 2028, 4985, 4799, 4850, 1103, 4757, 4747, 1614, 1947, 2237, 630, 631, 4997, 4618, 4479, 3766, 632, 1104, 1105, 4886, 1106, 2432, 4827, 633, 1107, 4530, 634, 2371, 1108, 4536, 635, 1813, 636, 2431, 4866, 4991, 4814, 4560, 4956, 637, 4555, 1109, 4941, 4633, 2213, 638, 639, 3706, 1110, 640, 1111, 641, 4581, 4893, 4831, 2481, 4644, 4734, 2190, 4524, 4881, 4837, 4888, 4648, 642, 4548, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4629, 1115, 4918, 4688, 3920, 644, 4723, 4949, 4748, 4607, 4719, 1116, 645, 646, 4611, 2353, 4603, 4762, 4819, 2221, 4636, 4992, 4998, 2307, 647, 1801, 4279, 3762, 1637, 1509, 4843, 4797, 4590, 4516, 4995, 4541, 2200, 4580, 4858, 648, 649, 2502, 2477, 4724, 4551, 4674, 1118, 1119, 2965, 2369, 2695, 3293, 650, 651, 4730, 4862, 4722, 4944, 1120, 652, 1121, 4983, 4784, 4877, 4643, 653, 654, 4909, 1122, 3264, 655, 2076, 4849, 656, 657, 2447, 658, 659, 2191, 4035, 2910, 3391, 2808, 3099, 2913, 3676, 2852, 3284, 2731, 1609, 2375, 2201, 3537, 3994, 1319, 4467, 1802, 1227, 1623, 660, 4844, 4714, 4979, 4812, 2192, 1123, 661, 2364, 662, 663, 664, 1124, 1125, 665, 2487, 4899, 2138, 1126, 666, 667, 4569, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 674, 2456, 4588, 4963, 4851, 4558, 4557, 4697, 4610, 4773, 4687, 4708, 4910, 2123, 1136, 1137, 2358, 5005, 2208, 4952, 4945, 1933, 1731, 1793, 675, 676, 3342, 1138, 1139, 677, 4840, 4597, 4870, 1140, 4990, 4954, 4916, 678, 1141, 4737, 4527, 2253, 4531, 679, 1142, 1143, 680, 4771, 1144, 4638, 681, 4705, 4525, 4522, 4898, 2325, 2415, 5004, 1145, 682, 683, 2141, 1146, 684, 685, 2445, 686, 687, 4861, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4690, 4859, 4793, 4575, 2160, 4828, 4731, 4718, 4802, 4750, 1150, 2081, 2258, 697, 1795, 1372, 1695, 1866, 1151, 4115, 1719, 2280, 4662, 698, 2176, 2217, 1920, 699, 700, 701, 4999, 1544, 4739, 2512, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 2340, 4182, 706, 4652, 2442, 2484, 707, 708, 709, 4667, 1153, 2412, 4732, 4476, 2053, 710, 1375, 711, 712, 713, 4829, 4738, 4811, 4570, 1245, 714, 2045, 4707, 715, 2169, 4713, 1841, 4234, 4924, 1154, 1314, 4096, 2023, 717, 4895, 2414, 718, 1155, 719, 720, 2063, 1311, 4921, 721, 4833, 4903, 4885, 4666, 2249, 4217, 1156, 723, 1157, 1627, 2116, 4566, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 2248, 730, 731, 1846, 2507, 2368, 1162, 2019, 2333, 3815, 2005, 2266, 1964, 2347, 732, 2177, 2059, 4958, 4927, 1822, 4815, 1262, 2094, 4480, 1537, 1291, 2419, 1318, 4628, 4294, 2486, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4679, 4835, 2259, 4839, 2275, 1166, 4968, 4912, 2423, 737, 1167, 4989, 4684, 4612, 2127, 1745, 1973, 4767, 1168, 2273, 2310, 3807, 4307, 1169, 2303, 1454, or 1781.

A VP capsid polypeptide may have a 581 to 589 region having a sequence of SEQ ID NO: 5014, wherein X⁶ of SEQ ID NO: 5014 is not D. In some embodiments, the 581 to 589 region has a sequence of any one of SEQ ID NOs: 2498, 4884, 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 2469, 4889, 21, 3192, 4942, 22, 23, 1638, 748, 24, 25, 749, 26, 27, 1703, 1836, 28, 2038, 2301, 3660, 29, 750, 30, 31, 2095, 1640, 33, 34, 35, 36, 37, 4742, 2450, 38, 4925, 39, 751, 752, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2072, 2034, 50, 1308, 1230, 3960, 1378, 2994, 3080, 2589, 3681, 51, 4694, 2257, 2167, 52, 760, 53, 2389, 4934, 4951, 4755, 761, 4591, 762, 4659, 54, 2146, 3925, 55, 3654, 2087, 2346, 4766, 4682, 4984, 4744, 56, 763, 764, 4626, 4702, 57, 58, 59, 60, 61, 4086, 1740, 1660, 765, 766, 62, 1966, 2032, 2499, 2332, 767, 63, 2096, 2181, 4913, 2438, 2041, 64, 768, 769, 770, 2384, 2402, 771, 65, 66, 67, 772, 2458, 2197, 68, 3816, 69, 2599, 2706, 2497, 70, 2234, 2183, 2399, 71, 4769, 2366, 72, 73, 74, 3865, 1549, 1361, 2453, 3882, 773, 3713, 1942, 2140, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2132, 81, 82, 4065, 83, 84, 2030, 1294, 2413, 2064, 1484, 2299, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 2356, 784, 785, 786, 94, 95, 787, 788, 1831, 1867, 96, 4458, 97, 1970, 4656, 2173, 1936, 2349, 1590, 4585, 98, 99, 100, 789, 790, 1298, 101, 1688, 791, 792, 2007, 4556, 2070, 102, 793, 4632, 103, 794, 795, 104, 105, 106, 2229, 796, 4691, 107, 2440, 1849, 108, 797, 4736, 2509, 2018, 2227, 4787, 109, 1579, 110, 1906, 111, 2428, 798, 2075, 112, 2411, 113, 2359, 799, 4848, 800, 2461, 1444, 2313, 2350, 4579, 4821, 801, 114, 2329, 4864, 4834, 115, 1344, 116, 802, 117, 118, 119, 4929, 4677, 120, 4791, 2158, 121, 803, 804, 1463, 805, 122, 2139, 2290, 1760, 2465, 123, 2424, 2058, 2230, 124, 4717, 806, 125, 126, 2370, 127, 4521, 128, 4971, 4994, 807, 4533, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 5009, 144, 145, 816, 4654, 2125, 4663, 4810, 4816, 1634, 1302, 817, 146, 818, 147, 148, 149, 4546, 150, 151, 152, 153, 154, 155, 819, 820, 156, 2149, 157, 2295, 821, 158, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 4809, 4520, 180, 826, 827, 181, 828, 4695, 2130, 4911, 182, 4582, 4873, 4596, 2020, 2189, 183, 4641, 829, 184, 185, 2244, 4647, 186, 187, 188, 189, 190, 830, 191, 192, 193, 194, 2071, 4615, 2361, 195, 4852, 4914, 4947, 196, 1391, 197, 4534, 4726, 198, 199, 4920, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2090, 2241, 217, 218, 4653, 219, 1612, 220, 221, 222, 839, 223, 4953, 4974, 4790, 4915, 2464, 4567, 4774, 4964, 2360, 840, 224, 4928, 4741, 225, 2088, 4751, 5007, 4542, 226, 227, 4890, 4519, 4883, 228, 4786, 229, 230, 231, 2448, 841, 842, 232, 233, 4782, 234, 4962, 2269, 843, 235, 844, 236, 237, 4930, 845, 846, 238, 847, 4704, 239, 848, 849, 2150, 850, 851, 852, 853, 1536, 240, 854, 241, 242, 243, 244, 245, 246, 247, 248, 4923, 2245, 4696, 855, 4634, 856, 249, 250, 251, 252, 253, 254, 255, 857, 2115, 2218, 256, 257, 258, 259, 858, 859, 260, 261, 860, 861, 862, 262, 4966, 863, 864, 263, 4749, 4970, 2296, 2318, 4535, 865, 2326, 264, 1241, 265, 266, 866, 1477, 267, 867, 268, 868, 269, 4668, 270, 869, 870, 271, 871, 872, 272, 4822, 4709, 873, 4655, 4972, 2317, 874, 1252, 273, 1591, 4975, 875, 274, 2025, 275, 1980, 276, 876, 877, 2066, 277, 278, 279, 4670, 280, 2323, 2288, 2334, 4939, 4689, 281, 878, 2416, 1892, 879, 1539, 2214, 2215, 880, 2327, 881, 882, 4601, 1295, 3601, 3877, 1930, 1420, 1678, 1460, 2306, 884, 282, 1799, 283, 284, 4988, 4871, 4553, 4772, 2260, 285, 1626, 885, 286, 1374, 886, 2119, 2504, 2468, 287, 2408, 1620, 1290, 2425, 2482, 4564, 4671, 2282, 288, 1354, 2033, 289, 887, 290, 888, 291, 1956, 2362, 2467, 889, 2357, 4547, 4765, 292, 4794, 2391, 4324, 293, 890, 294, 295, 4138, 891, 2297, 2254, 296, 2224, 3903, 297, 892, 3829, 298, 299, 3809, 4271, 3844, 1436, 300, 1515, 1617, 4701, 2174, 301, 1961, 4649, 5010, 302, 2060, 303, 304, 305, 2452, 2147, 4574, 4538, 306, 2293, 1589, 893, 4946, 4370, 4841, 2342, 307, 308, 309, 894, 2246, 2155, 3646, 4059, 1431, 1331, 311, 4836, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 1635, 4820, 905, 331, 4706, 2092, 4484, 1756, 906, 907, 4661, 2179, 4584, 4609, 332, 4907, 4635, 4529, 4683, 4561, 333, 2337, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4783, 4319, 3906, 1533, 1755, 1888, 4248, 1557, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 2444, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 2363, 908, 1935, 4711, 4728, 2460, 4517, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 337, 1184, 3450, 2577, 2567, 2532, 3415, 2934, 2984, 2568, 4147, 4943, 3002, 2986, 910, 2979, 2542, 2338, 2331, 2492, 1762, 4472, 1472, 1529, 2319, 4057, 4937, 2048, 4887, 1976, 4507, 1583, 338, 2078, 2372, 2102, 339, 911, 1711, 340, 912, 2013, 913, 2277, 914, 2403, 341, 915, 4902, 916, 342, 1594, 4440, 917, 343, 344, 4559, 918, 2233, 2439, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 2471, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 371, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2343, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 2383, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2129, 2345, 4471, 952, 953, 401, 2475, 403, 404, 4758, 954, 955, 2437, 4139, 1482, 3602, 4434, 3721, 1592, 1825, 1698, 1519, 1352, 4202, 4409, 1977, 1917, 405, 406, 407, 408, 956, 2272, 957, 2267, 1631, 1684, 409, 2054, 3889, 958, 410, 2117, 411, 959, 412, 960, 2443, 4712, 4619, 2285, 413, 414, 415, 961, 2352, 2417, 962, 1905, 416, 963, 417, 418, 2382, 3675, 1833, 3974, 419, 964, 420, 421, 965, 966, 967, 968, 2365, 969, 970, 422, 1897, 2056, 2309, 4733, 1272, 4754, 2207, 971, 423, 4133, 3830, 4865, 972, 424, 973, 425, 4955, 426, 974, 2109, 2380, 427, 1708, 4676, 428, 4141, 429, 430, 3749, 431, 1619, 975, 4044, 3590, 1944, 4126, 1923, 1706, 432, 976, 977, 978, 4867, 4400, 433, 434, 1606, 979, 435, 980, 2604, 2895, 981, 436, 3448, 4959, 4563, 2188, 437, 438, 2055, 1692, 982, 2388, 2433, 2243, 983, 2035, 2120, 4940, 439, 440, 441, 2474, 442, 4900, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 2242, 453, 2286, 992, 1728, 993, 4703, 994, 995, 454, 1696, 996, 2039, 997, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 466, 2328, 467, 468, 469, 1231, 1468, 470, 471, 2496, 472, 473, 1005, 474, 2108, 4725, 1006, 476, 1273, 1007, 1744, 1488, 1659, 477, 478, 4598, 1604, 4753, 2493, 479, 1008, 1009, 1010, 1011, 1012, 1013, 1014, 481, 4313, 482, 483, 1499, 484, 1015, 4761, 2091, 2386, 2513, 1016, 4073, 1777, 2172, 485, 2205, 4592, 4545, 4720, 486, 1458, 4274, 2122, 4905, 5003, 4599, 4796, 2152, 4539, 4528, 4904, 4842, 4986, 4639, 4846, 4623, 4650, 4595, 2195, 4681, 2142, 487, 2182, 4710, 1775, 2083, 4205, 1526, 4586, 4602, 4518, 1697, 1017, 2000, 4692, 2480, 2017, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 2202, 5001, 2136, 493, 2211, 4532, 494, 2421, 495, 4090, 2378, 2210, 2247, 2478, 2047, 496, 4620, 4978, 1019, 2012, 1803, 3642, 497, 1322, 4752, 1020, 498, 1021, 2235, 2302, 2488, 4665, 4540, 5006, 4699, 4608, 4781, 499, 2049, 2404, 4685, 500, 501, 1022, 502, 1333, 503, 1023, 4686, 4804, 504, 4727, 505, 506, 4805, 2065, 1890, 1024, 507, 508, 509, 2110, 1025, 510, 1026, 3608, 1732, 511, 2397, 1864, 1816, 1531, 512, 513, 2451, 3636, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 4746, 2489, 4922, 2145, 1030, 4832, 4950, 1261, 4027, 3478, 3763, 2959, 1293, 2462, 517, 3197, 1595, 3413, 2238, 2067, 1031, 2228, 1448, 518, 4698, 4616, 2057, 2305, 2133, 2061, 4526, 4617, 1032, 1033, 2429, 2311, 1675, 4785, 1797, 519, 4578, 2271, 4693, 4604, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 1038, 536, 1039, 537, 538, 539, 1040, 3767, 1929, 1041, 1042, 3683, 2100, 4853, 2300, 2165, 540, 2105, 541, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1649, 543, 544, 545, 546, 1045, 547, 2084, 2225, 4240, 3580, 4680, 1046, 548, 2062, 549, 1047, 550, 2598, 551, 552, 553, 3819, 1259, 4062, 1048, 554, 2841, 1049, 1050, 3515, 1433, 1323, 4301, 555, 556, 1051, 557, 558, 1052, 1053, 3738, 1567, 559, 1054, 1270, 3857, 1055, 2151, 560, 2175, 2089, 4514, 2198, 2193, 1257, 1238, 3662, 561, 2186, 1057, 562, 1058, 563, 4872, 2097, 2436, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1705, 1861, 1197, 1254, 4120, 1545, 4417, 1469, 1292, 2185, 1785, 1236, 4293, 1827, 3839, 3983, 4395, 1957, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 2304, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2255, 2113, 590, 591, 1085, 2256, 592, 2308, 593, 594, 595, 4931, 2407, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 2321, 1086, 601, 4017, 4477, 1794, 2483, 4776, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 2320, 2069, 610, 611, 1093, 612, 1094, 3701, 4389, 613, 4897, 1603, 614, 1095, 2154, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4801, 4855, 4847, 2398, 2239, 1101, 1667, 1384, 623, 4745, 3700, 1788, 1600, 624, 2098, 625, 4550, 4721, 4960, 2473, 4792, 4675, 1102, 1423, 626, 2771, 627, 1339, 3875, 628, 629, 3070, 4565, 2118, 2015, 4818, 4860, 2557, 2795, 2906, 2103, 4869, 2029, 3299, 3118, 4932, 4838, 4901, 4908, 4996, 4779, 2377, 4657, 4646, 4875, 4627, 4515, 4876, 4965, 4605, 4576, 2028, 4985, 4799, 4850, 1103, 4757, 4747, 1614, 1947, 2237, 630, 631, 4997, 4618, 4479, 3766, 632, 1104, 1105, 4886, 1106, 2432, 4827, 633, 1107, 4530, 634, 2371, 4651, 1108, 4536, 635, 1813, 636, 2431, 4866, 4991, 4814, 4560, 4956, 637, 4555, 1109, 4577, 4633, 2213, 638, 639, 3706, 1110, 640, 1111, 641, 4581, 4893, 4831, 2481, 4644, 4734, 2190, 4881, 4837, 4888, 4648, 642, 4548, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4760, 4629, 1115, 4918, 4688, 3920, 644, 4723, 4949, 4607, 4719, 1116, 645, 646, 4611, 2353, 4645, 4603, 4762, 4819, 2221, 4636, 4992, 2307, 647, 1801, 4279, 3762, 1637, 1509, 4843, 4797, 4590, 4516, 4541, 2200, 4580, 4858, 648, 649, 1117, 4780, 4800, 2477, 4724, 4551, 4674, 1118, 1119, 2965, 2369, 2695, 3293, 650, 651, 4862, 4722, 4944, 1120, 652, 1121, 4983, 4784, 4877, 653, 654, 1122, 3264, 655, 2076, 4849, 656, 657, 2447, 658, 659, 2191, 4035, 2910, 3391, 2808, 3099, 2913, 3676, 2852, 3284, 2731, 1609, 2375, 3537, 3994, 1319, 4467, 1802, 1227, 1623, 660, 4844, 2086, 4770, 4714, 4979, 4812, 2192, 1123, 661, 2364, 662, 663, 664, 1124, 1125, 665, 2487, 4899, 2138, 1126, 666, 667, 4569, 4554, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 674, 2456, 4606, 4588, 4963, 4851, 4558, 4557, 4697, 4610, 4773, 4687, 4708, 4910, 2123, 1136, 1137, 2358, 5005, 2208, 4952, 4945, 1933, 1731, 1793, 675, 676, 3342, 1138, 1139, 677, 4840, 4597, 4870, 1140, 4990, 4954, 4916, 678, 1141, 4737, 4527, 2253, 4531, 679, 1142, 1143, 680, 4771, 1144, 4638, 681, 4705, 4525, 4898, 2325, 2415, 5004, 1145, 682, 683, 2141, 1146, 684, 685, 2445, 686, 687, 4861, 2157, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4690, 4859, 4793, 4575, 2160, 4828, 4731, 4802, 4750, 1150, 2081, 2258, 697, 1795, 1372, 1695, 1866, 1151, 4115, 1719, 2280, 4662, 698, 2176, 2194, 2217, 1920, 699, 700, 701, 4999, 1544, 4739, 2512, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 4824, 705, 2340, 4182, 706, 4652, 2442, 2484, 707, 708, 709, 4667, 1153, 2412, 4732, 4476, 2053, 710, 1375, 711, 712, 713, 4738, 4811, 4570, 4961, 1245, 714, 2045, 4707, 715, 2169, 4713, 4879, 1841, 4234, 4924, 1154, 1314, 4096, 2023, 716, 717, 4895, 2414, 718, 1155, 719, 720, 2063, 1311, 4921, 721, 4642, 722, 4833, 4903, 4885, 4666, 2249, 4217, 1156, 723, 1157, 1627, 2116, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 2248, 730, 731, 2024, 1846, 2507, 2368, 1162, 2019, 2333, 3815, 2005, 2266, 1964, 2347, 2385, 732, 2177, 2059, 4958, 4927, 1822, 4815, 1262, 2094, 4480, 1537, 1291, 2419, 1318, 2283, 4976, 4628, 4294, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4835, 2259, 4571, 2275, 1166, 4968, 2250, 2423, 737, 1167, 4989, 4669, 4552, 4612, 2127, 1745, 1973, 4767, 1168, 2273, 2310, 3807, 4307, 1169, 2303, 1454, or 1781.

A VP capsid polypeptide may have a 581 to 589 region having a sequence of SEQ ID NO: 5014, wherein X³ of SEQ ID NO: 5014 is not D and X⁶ of SEQ ID NO: 5014 is not D. In some embodiments, the 581 to 589 region has a sequence of any one of SEQ ID NOs: 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 2469, 4889, 21, 3192, 4942, 22, 23, 1638, 748, 24, 25, 749, 26, 27, 1703, 1836, 28, 2038, 2301, 3660, 29, 750, 30, 31, 2095, 1640, 33, 34, 35, 36, 37, 4742, 2450, 38, 4925, 39, 751, 752, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2072, 2034, 50, 1308, 1230, 3960, 1378, 2994, 3080, 2589, 3681, 51, 2167, 52, 760, 53, 2389, 4755, 761, 4591, 762, 4659, 54, 2146, 3925, 55, 3654, 2087, 2346, 4766, 4682, 4984, 4744, 56, 763, 764, 4626, 4702, 57, 58, 59, 60, 61, 4086, 1740, 1660, 765, 766, 62, 1966, 2032, 2499, 2332, 767, 63, 2096, 2181, 4913, 2438, 2041, 64, 768, 769, 770, 2384, 2402, 771, 65, 66, 67, 772, 2458, 2197, 68, 3816, 69, 2599, 2706, 2497, 70, 2399, 71, 4769, 2366, 72, 73, 74, 3865, 1549, 1361, 2453, 3882, 773, 3713, 1942, 2140, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 81, 82, 4065, 83, 84, 2030, 1294, 2413, 2064, 1484, 2299, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 2356, 784, 785, 786, 94, 95, 788, 1831, 1867, 96, 4458, 97, 1970, 4656, 2173, 1936, 2349, 1590, 98, 99, 100, 789, 790, 1298, 101, 1688, 791, 792, 2007, 4556, 2070, 102, 793, 4632, 103, 794, 795, 104, 105, 106, 2229, 796, 4691, 107, 2440, 1849, 108, 797, 4736, 2509, 2018, 2227, 4787, 1579, 110, 1906, 111, 2428, 798, 2075, 112, 2411, 113, 2359, 799, 4848, 800, 2461, 1444, 2313, 2350, 4579, 4821, 801, 114, 2329, 4864, 4834, 115, 1344, 116, 802, 117, 118, 119, 4929, 4677, 120, 4791, 2158, 121, 803, 804, 1463, 805, 122, 2139, 2290, 1760, 2465, 123, 2424, 2058, 2230, 124, 4717, 806, 125, 126, 2370, 127, 4521, 128, 4971, 4994, 807, 4533, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 5009, 144, 145, 816, 4654, 2125, 4663, 4810, 4816, 1634, 1302, 817, 146, 818, 147, 148, 149, 4546, 150, 151, 152, 153, 154, 155, 819, 820, 156, 2149, 157, 2295, 821, 158, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 4809, 4520, 180, 826, 827, 181, 828, 4695, 2130, 4911, 182, 4582, 4873, 2189, 183, 4641, 829, 184, 185, 2244, 4647, 186, 187, 188, 189, 190, 830, 191, 192, 193, 194, 2071, 4615, 2361, 195, 4852, 4914, 4947, 196, 1391, 197, 4534, 4726, 198, 199, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2090, 2241, 217, 218, 4653, 219, 1612, 220, 221, 222, 839, 223, 4974, 4790, 4915, 2464, 4567, 4774, 4964, 2360, 840, 224, 4928, 4741, 225, 2088, 4751, 226, 227, 4890, 4519, 4883, 228, 4786, 229, 230, 231, 2448, 841, 842, 232, 233, 4782, 234, 2269, 843, 235, 844, 236, 237, 4930, 845, 846, 238, 847, 239, 848, 849, 2150, 850, 851, 852, 853, 1536, 240, 854, 241, 242, 243, 244, 245, 246, 247, 248, 4696, 855, 4634, 856, 249, 250, 251, 252, 253, 254, 255, 857, 2115, 2218, 256, 257, 258, 259, 858, 859, 260, 261, 860, 861, 862, 262, 4966, 863, 864, 263, 4749, 4970, 2296, 2318, 4535, 865, 2326, 264, 1241, 265, 266, 866, 1477, 267, 867, 268, 868, 269, 4668, 270, 869, 870, 271, 871, 872, 272, 4822, 4709, 873, 4655, 4972, 2317, 874, 1252, 273, 1591, 4975, 875, 274, 2025, 275, 1980, 276, 876, 877, 2066, 277, 278, 279, 4670, 280, 2323, 2288, 2334, 4939, 4689, 281, 878, 2416, 1892, 879, 1539, 2214, 2215, 880, 2327, 881, 882, 4601, 1295, 3601, 3877, 1930, 1420, 1678, 1460, 2306, 884, 282, 1799, 283, 284, 4988, 4871, 4553, 4772, 2260, 1626, 885, 286, 1374, 886, 2119, 2504, 2468, 287, 1620, 1290, 2425, 2482, 4564, 4671, 2282, 288, 1354, 2033, 289, 887, 290, 888, 291, 1956, 2362, 2467, 889, 2357, 4547, 4765, 292, 4794, 2391, 4324, 293, 890, 294, 295, 4138, 891, 2297, 2254, 296, 2224, 3903, 297, 892, 3829, 298, 299, 3809, 4271, 3844, 1436, 300, 1515, 1617, 301, 1961, 4649, 5010, 302, 2060, 303, 304, 305, 2452, 2147, 4574, 4538, 306, 2293, 1589, 893, 4946, 4370, 4841, 2342, 307, 308, 309, 894, 2246, 2155, 3646, 4059, 1431, 1331, 311, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 1635, 4820, 905, 331, 4706, 4484, 1756, 906, 907, 4661, 2179, 4584, 4609, 332, 4907, 4635, 4529, 4683, 4561, 333, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4319, 3906, 1533, 1755, 1888, 4248, 1557, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 2363, 908, 1935, 4711, 4728, 2460, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 337, 1184, 3450, 2577, 2567, 2532, 3415, 2934, 2984, 2568, 4147, 4943, 3002, 2986, 910, 2979, 2542, 2338, 2331, 2492, 1762, 4472, 1472, 1529, 2319, 4057, 4937, 2048, 4887, 1976, 4507, 1583, 338, 2078, 2372, 2102, 339, 911, 1711, 340, 912, 2013, 913, 2277, 914, 2403, 341, 915, 4902, 916, 342, 1594, 4440, 917, 343, 344, 4559, 918, 2439, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 371, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2345, 4471, 952, 953, 401, 2475, 403, 404, 4758, 954, 955, 2437, 4139, 1482, 3602, 4434, 3721, 1592, 1825, 1698, 1519, 1352, 4202, 4409, 1977, 1917, 405, 406, 407, 408, 956, 957, 2267, 1631, 1684, 409, 2054, 3889, 958, 410, 2117, 411, 959, 412, 960, 2443, 4712, 4619, 2285, 413, 414, 415, 961, 2352, 2417, 962, 1905, 416, 963, 417, 418, 2382, 3675, 1833, 3974, 419, 964, 420, 421, 965, 966, 967, 968, 2365, 969, 970, 422, 1897, 2056, 2309, 1272, 4754, 2207, 971, 423, 4133, 3830, 4865, 972, 424, 973, 425, 4955, 426, 974, 2109, 2380, 427, 1708, 4676, 428, 4141, 429, 430, 3749, 431, 1619, 975, 4044, 3590, 1944, 4126, 1923, 1706, 432, 976, 977, 978, 4867, 4400, 433, 434, 1606, 979, 435, 980, 2604, 2895, 981, 436, 3448, 4959, 4563, 2188, 437, 438, 2055, 1692, 982, 2388, 2243, 983, 2035, 2120, 4940, 439, 440, 441, 2474, 4900, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 2242, 453, 2286, 992, 1728, 993, 4703, 994, 995, 454, 1696, 996, 2039, 997, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 466, 2328, 467, 468, 469, 1231, 1468, 470, 471, 2496, 472, 473, 1005, 474, 2108, 4725, 1006, 476, 1273, 1007, 1744, 1488, 1659, 477, 478, 4598, 1604, 4753, 2493, 479, 1008, 1009, 1010, 1011, 1012, 1013, 1014, 481, 4313, 482, 483, 1499, 484, 1015, 4761, 2091, 2386, 2513, 1016, 4073, 1777, 2172, 485, 2205, 4592, 4545, 4720, 486, 1458, 4274, 2122, 4905, 4796, 2152, 4539, 4528, 4904, 4842, 4986, 4639, 4846, 4623, 4650, 4595, 2195, 4681, 487, 2182, 4710, 1775, 2083, 4205, 1526, 4586, 1697, 1017, 2000, 4692, 2480, 2017, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 5001, 2136, 493, 2211, 4532, 494, 2421, 495, 4090, 2378, 2210, 2247, 2478, 2047, 496, 4620, 4978, 1019, 2012, 1803, 3642, 497, 1322, 4752, 1020, 498, 1021, 2235, 2302, 2488, 4665, 4699, 4608, 4781, 499, 2049, 2404, 4685, 500, 501, 1022, 502, 1333, 503, 1023, 4686, 4804, 504, 4727, 505, 506, 4805, 2065, 1890, 1024, 507, 508, 509, 2110, 1025, 510, 1026, 3608, 1732, 511, 2397, 1864, 1816, 1531, 512, 513, 2451, 3636, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 4746, 2489, 4922, 2145, 1030, 4832, 4950, 1261, 4027, 3478, 3763, 2959, 1293, 2462, 517, 3197, 1595, 3413, 2067, 1031, 2228, 1448, 518, 4698, 4616, 2057, 2305, 2133, 2061, 4526, 4617, 1032, 1033, 2429, 2311, 1675, 1797, 519, 4578, 2271, 4693, 4604, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 1038, 536, 1039, 537, 538, 539, 1040, 3767, 1929, 1041, 1042, 3683, 2100, 4853, 2300, 2165, 540, 2105, 541, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1649, 543, 544, 545, 546, 1045, 547, 2225, 4240, 3580, 4680, 1046, 548, 549, 1047, 550, 2598, 551, 552, 553, 3819, 1259, 4062, 1048, 554, 2841, 1049, 1050, 3515, 1433, 1323, 4301, 555, 556, 1051, 557, 558, 1052, 1053, 3738, 1567, 559, 1054, 1270, 3857, 1055, 2151, 560, 2175, 2089, 4514, 2198, 2193, 1257, 1238, 3662, 561, 2186, 1057, 562, 1058, 563, 4872, 2097, 2436, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 3934, 4080, 4352, 574, 1700, 3739, 1565, 1419, 1811, 3950, 1690, 1397, 3716, 1569, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 575, 4443, 3761, 1346, 1587, 1072, 3592, 576, 1891, 4469, 1275, 577, 1950, 1963, 4129, 1073, 1296, 1360, 1551, 3733, 4111, 3993, 4108, 3949, 1287, 1713, 1556, 1736, 1995, 1435, 1424, 1705, 1861, 1197, 1254, 4120, 1545, 4417, 1469, 1292, 2185, 1785, 1236, 4293, 1827, 3839, 3983, 4395, 1957, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 2304, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2113, 590, 591, 1085, 2256, 592, 2308, 593, 594, 595, 4931, 2407, 1858, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 2321, 1086, 601, 4017, 4477, 1794, 2483, 4776, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 2320, 2069, 610, 611, 1093, 612, 1094, 3701, 4389, 613, 4897, 1603, 614, 1095, 2154, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4855, 4847, 2398, 2239, 1101, 1667, 1384, 623, 4745, 3700, 1788, 1600, 624, 2098, 625, 4550, 4721, 4960, 2473, 4792, 4675, 1102, 1423, 626, 2771, 627, 1339, 3875, 628, 629, 3070, 4565, 2118, 2015, 4818, 4860, 2557, 2795, 2906, 2103, 4869, 2029, 3299, 3118, 4932, 4838, 4901, 4908, 4996, 4779, 2377, 4657, 4646, 4875, 4627, 4515, 4876, 4965, 4605, 4576, 2028, 4985, 4799, 4850, 1103, 4757, 4747, 1614, 1947, 2237, 630, 631, 4997, 4618, 4479, 3766, 632, 1104, 1105, 4886, 1106, 2432, 4827, 633, 1107, 4530, 634, 2371, 1108, 4536, 635, 1813, 636, 2431, 4866, 4991, 4814, 4560, 4956, 637, 4555, 1109, 4633, 2213, 638, 639, 3706, 1110, 640, 1111, 641, 4581, 4893, 4831, 2481, 4644, 4734, 2190, 4881, 4837, 4888, 4648, 642, 4548, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4629, 1115, 4918, 4688, 3920, 644, 4723, 4949, 4607, 4719, 1116, 645, 646, 4611, 2353, 4603, 4762, 4819, 2221, 4636, 4992, 2307, 647, 1801, 4279, 3762, 1637, 1509, 4843, 4797, 4590, 4516, 4541, 2200, 4580, 4858, 648, 649, 2477, 4724, 4551, 4674, 1118, 1119, 2965, 2369, 2695, 3293, 650, 651, 4862, 4722, 4944, 1120, 652, 1121, 4983, 4784, 4877, 653, 654, 1122, 3264, 655, 2076, 4849, 656, 657, 2447, 658, 659, 2191, 4035, 2910, 3391, 2808, 3099, 2913, 3676, 2852, 3284, 2731, 1609, 2375, 3537, 3994, 1319, 4467, 1802, 1227, 1623, 660, 4844, 4714, 4979, 4812, 2192, 1123, 661, 2364, 662, 663, 664, 1124, 1125, 665, 2487, 4899, 2138, 1126, 666, 667, 4569, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 674, 2456, 4588, 4963, 4851, 4558, 4557, 4697, 4610, 4773, 4687, 4708, 4910, 2123, 1136, 1137, 2358, 5005, 2208, 4952, 4945, 1933, 1731, 1793, 675, 676, 3342, 1138, 1139, 677, 4840, 4597, 4870, 1140, 4990, 4954, 4916, 678, 1141, 4737, 4527, 2253, 4531, 679, 1142, 1143, 680, 4771, 1144, 4638, 681, 4705, 4525, 4898, 2325, 2415, 5004, 1145, 682, 683, 2141, 1146, 684, 685, 2445, 686, 687, 4861, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4690, 4859, 4793, 4575, 2160, 4828, 4731, 4802, 4750, 1150, 2081, 2258, 697, 1795, 1372, 1695, 1866, 1151, 4115, 1719, 2280, 4662, 698, 2176, 2217, 1920, 699, 700, 701, 4999, 1544, 4739, 2512, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 2340, 4182, 706, 4652, 2442, 2484, 707, 708, 709, 4667, 1153, 2412, 4732, 4476, 2053, 710, 1375, 711, 712, 713, 4738, 4811, 4570, 1245, 714, 2045, 4707, 715, 2169, 4713, 1841, 4234, 4924, 1154, 1314, 4096, 2023, 717, 4895, 2414, 718, 1155, 719, 720, 2063, 1311, 4921, 721, 4833, 4903, 4885, 4666, 2249, 4217, 1156, 723, 1157, 1627, 2116, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 2248, 730, 731, 1846, 2507, 2368, 1162, 2019, 2333, 3815, 2005, 2266, 1964, 2347, 732, 2177, 2059, 4958, 4927, 1822, 4815, 1262, 2094, 4480, 1537, 1291, 2419, 1318, 4628, 4294, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4835, 2259, 2275, 1166, 4968, 2423, 737, 1167, 4989, 4612, 2127, 1745, 1973, 4767, 1168, 2273, 2310, 3807, 4307, 1169, 2303, 1454, or 1781.

A VP capsid polypeptide may have a 581 to 589 region having a sequence of SEQ ID NO: 5014, wherein X³ of SEQ ID NO: 5014 is not E. In some embodiments, the 581 to 589 region has a sequence of any one of SEQ ID NOs: 2498, 4884, 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 4889, 21, 3192, 4942, 22, 23, 1638, 748, 24, 25, 749, 26, 27, 1703, 1836, 28, 2038, 2301, 3660, 29, 750, 30, 31, 32, 2095, 1640, 33, 34, 35, 36, 37, 4742, 2450, 38, 4925, 39, 751, 752, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2080, 2072, 2034, 50, 1308, 1230, 3960, 1378, 2994, 3080, 2589, 3681, 51, 4694, 2257, 52, 760, 53, 2389, 4934, 4951, 761, 4591, 762, 4659, 54, 2146, 3925, 55, 3654, 2087, 2346, 4766, 4682, 4984, 4744, 56, 763, 764, 4626, 4702, 57, 58, 59, 60, 61, 4086, 1740, 1660, 765, 766, 62, 1966, 2032, 2499, 2332, 767, 63, 2096, 2181, 4913, 64, 768, 769, 770, 2384, 2402, 771, 65, 66, 2111, 67, 772, 2458, 2197, 68, 3816, 69, 2599, 2706, 2497, 70, 2234, 2183, 2399, 71, 4769, 2366, 72, 73, 74, 3865, 1549, 1361, 2453, 3882, 773, 3713, 1942, 2140, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2132, 2379, 81, 82, 4065, 83, 84, 2030, 1294, 2413, 2064, 1484, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 93, 2356, 784, 785, 786, 94, 95, 787, 788, 1831, 1867, 96, 4458, 97, 1970, 4656, 2173, 1936, 2349, 1590, 4585, 98, 99, 100, 789, 790, 1298, 101, 1688, 791, 792, 2007, 4556, 2070, 102, 793, 4632, 103, 794, 795, 104, 105, 106, 2229, 796, 4980, 4691, 107, 2440, 1849, 108, 2014, 797, 4736, 2509, 2018, 2227, 4787, 109, 1579, 110, 1906, 111, 2428, 798, 2075, 112, 2411, 113, 2359, 799, 4848, 800, 2461, 1444, 2313, 2350, 4579, 4821, 801, 114, 2329, 4834, 115, 1344, 116, 802, 117, 118, 119, 4929, 4677, 120, 2158, 121, 803, 804, 1463, 805, 122, 2139, 2290, 1760, 2465, 123, 2424, 2058, 2230, 124, 4717, 806, 125, 126, 2370, 127, 4521, 128, 4971, 4994, 807, 2085, 4533, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 4544, 5009, 144, 145, 816, 4654, 4816, 1634, 1302, 817, 146, 818, 147, 148, 149, 4546, 150, 151, 152, 153, 154, 155, 819, 820, 156, 2149, 157, 2295, 821, 158, 159, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 2170, 4809, 4520, 180, 826, 827, 181, 828, 4695, 4911, 182, 4582, 4873, 4596, 2020, 183, 4641, 829, 184, 185, 2244, 4647, 186, 187, 188, 189, 190, 2114, 830, 191, 192, 193, 194, 2071, 4615, 2361, 195, 4852, 2187, 4914, 4947, 196, 1391, 197, 4534, 4726, 4788, 198, 199, 4920, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2241, 217, 218, 4653, 219, 1612, 220, 221, 222, 839, 223, 4953, 2360, 840, 224, 4928, 4741, 225, 2088, 4751, 5007, 4542, 226, 227, 4890, 4519, 4883, 228, 4786, 229, 230, 231, 2393, 2448, 841, 842, 232, 233, 4782, 234, 4962, 2269, 843, 235, 844, 236, 237, 4930, 845, 846, 238, 847, 4704, 239, 848, 849, 2150, 850, 851, 852, 853, 1536, 240, 854, 241, 242, 243, 244, 245, 246, 247, 248, 4923, 2245, 4696, 855, 4634, 856, 249, 250, 251, 252, 253, 254, 255, 857, 2115, 4880, 2218, 256, 257, 258, 259, 858, 859, 260, 261, 860, 861, 862, 262, 4966, 863, 864, 263, 4749, 4970, 2296, 2318, 865, 2326, 264, 1241, 265, 266, 866, 1477, 267, 867, 4977, 268, 868, 269, 4668, 5002, 270, 869, 870, 271, 871, 872, 272, 4822, 4709, 873, 4655, 4972, 2392, 874, 1252, 273, 1591, 4975, 875, 274, 2025, 275, 1980, 276, 876, 877, 2066, 277, 278, 279, 4670, 280, 2323, 2288, 2334, 4939, 4689, 281, 878, 1892, 879, 1539, 2214, 2215, 880, 2327, 881, 882, 4601, 883, 1295, 3601, 3877, 1930, 1420, 1678, 1460, 2306, 884, 282, 1799, 283, 284, 4988, 4553, 4772, 4817, 2260, 285, 1626, 2082, 885, 286, 1374, 886, 2119, 2468, 287, 2408, 1620, 1290, 2425, 2482, 4564, 4671, 2282, 288, 2354, 1354, 2033, 289, 887, 290, 888, 291, 1956, 2362, 2467, 889, 2357, 4547, 4765, 292, 4794, 2391, 4324, 293, 890, 294, 295, 4138, 891, 2297, 2254, 296, 2224, 3903, 297, 892, 3829, 298, 299, 3809, 4271, 3844, 1436, 300, 1515, 1617, 4701, 2174, 301, 1961, 4649, 5010, 302, 303, 304, 305, 2452, 2147, 306, 2293, 1589, 893, 4370, 4841, 2342, 307, 308, 309, 894, 2246, 2155, 3646, 4059, 1431, 1331, 310, 311, 4836, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 904, 1635, 4820, 905, 2506, 331, 4706, 2092, 4484, 1756, 906, 907, 4661, 2179, 4584, 4609, 332, 4907, 4635, 4529, 4683, 4523, 4561, 333, 2337, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4783, 4319, 3906, 1533, 1755, 1888, 4248, 1557, 2153, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 2444, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 2363, 908, 1935, 4711, 4728, 2460, 4517, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 910, 2979, 2542, 2338, 2492, 1762, 4472, 1472, 1529, 2319, 2131, 4057, 4937, 2048, 1976, 4507, 1583, 338, 2078, 2372, 339, 911, 1711, 340, 912, 2013, 913, 2277, 914, 2403, 341, 915, 4672, 4902, 916, 342, 1594, 4440, 917, 343, 344, 4559, 918, 2233, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 2471, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 371, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2343, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 2383, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2129, 2345, 4471, 952, 953, 402, 2475, 403, 404, 4758, 955, 2437, 4139, 1482, 3602, 4434, 3721, 1592, 1825, 1698, 1519, 1352, 4202, 4409, 1977, 1917, 405, 406, 407, 408, 956, 2272, 957, 2267, 1631, 1684, 409, 2054, 3889, 958, 410, 2117, 411, 959, 412, 960, 2443, 4712, 4619, 2285, 414, 415, 961, 2352, 2417, 962, 1905, 416, 963, 417, 418, 3675, 1833, 3974, 419, 964, 420, 421, 965, 966, 967, 968, 2365, 969, 970, 422, 1897, 2056, 2309, 4733, 1272, 4754, 2207, 971, 423, 4133, 3830, 4865, 972, 424, 973, 425, 2068, 4955, 426, 974, 427, 1708, 4676, 428, 4141, 430, 3749, 431, 1619, 975, 4044, 3590, 1944, 4126, 1923, 1706, 432, 976, 977, 978, 4867, 4400, 433, 434, 1606, 2143, 979, 435, 980, 2604, 2895, 981, 436, 3448, 4959, 4563, 2188, 437, 438, 1692, 982, 2388, 2433, 2243, 983, 2035, 2120, 4940, 439, 440, 441, 2474, 442, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 2242, 453, 2286, 992, 1728, 993, 4703, 994, 995, 454, 1696, 996, 2039, 455, 997, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 467, 468, 469, 1231, 1468, 470, 471, 2496, 472, 473, 1005, 474, 475, 2108, 4725, 1006, 476, 1273, 1007, 1744, 1488, 1659, 477, 478, 4598, 1604, 4753, 2493, 479, 1008, 1009, 480, 1010, 1011, 1012, 1013, 1014, 481, 4313, 482, 483, 1499, 484, 1015, 4761, 2091, 2513, 1016, 4073, 1777, 2172, 485, 2205, 4592, 4545, 4720, 486, 1458, 4274, 2122, 4905, 5003, 4599, 4796, 2152, 4539, 4528, 4904, 4842, 4986, 4639, 4846, 4623, 4650, 4595, 2195, 4681, 2142, 487, 2182, 4710, 1775, 2083, 4205, 1526, 4586, 4602, 4518, 1697, 1017, 2000, 4692, 4795, 2480, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 2202, 5001, 2136, 493, 2211, 4532, 494, 2421, 495, 4090, 2378, 2104, 2210, 2247, 2478, 2047, 496, 4620, 4978, 4614, 1019, 2012, 1803, 3642, 497, 1322, 1020, 498, 1021, 2235, 2302, 2488, 4665, 4540, 5006, 499, 2049, 2404, 4685, 500, 501, 1022, 502, 1333, 503, 1023, 4686, 4804, 504, 4727, 505, 506, 4938, 4805, 2065, 1890, 4891, 1024, 507, 508, 509, 1025, 510, 1026, 3608, 1732, 511, 2397, 1864, 1816, 1531, 512, 513, 2451, 3636, 5012, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 2093, 4746, 2489, 4922, 2145, 1030, 4832, 4950, 1261, 4027, 3478, 3763, 2959, 1293, 2462, 517, 3197, 1595, 3413, 2238, 1031, 2228, 1448, 518, 2057, 2305, 2133, 2061, 4526, 4617, 1033, 2429, 2311, 1675, 4785, 1797, 519, 4578, 2271, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 535, 1038, 536, 1039, 537, 538, 539, 1040, 3767, 1929, 1041, 1042, 3683, 2100, 4853, 2300, 2165, 540, 541, 1043, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1649, 543, 544, 545, 546, 1045, 547, 2084, 4240, 3580, 4680, 1046, 548, 2062, 1047, 550, 2598, 551, 552, 553, 3819, 1259, 4062, 1048, 554, 2841, 1049, 1050, 3515, 2219, 1433, 1323, 4301, 555, 556, 1051, 557, 558, 1052, 1053, 3738, 1567, 559, 1054, 1270, 3857, 1055, 2151, 560, 2175, 2089, 1056, 2198, 2193, 1257, 1238, 3662, 561, 2186, 1057, 4637, 562, 1058, 563, 4872, 2097, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 2304, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2255, 2113, 590, 591, 1085, 2256, 592, 2308, 593, 594, 2274, 595, 4931, 2407, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 2321, 1086, 601, 4017, 4477, 1794, 2483, 4776, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 610, 611, 1093, 612, 1094, 3701, 4389, 613, 4897, 1603, 614, 1095, 2154, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4801, 2398, 2239, 1101, 1667, 1384, 623, 4745, 3700, 1788, 1600, 624, 2098, 625, 4550, 4721, 4960, 4878, 2473, 4792, 4675, 1102, 1423, 626, 2771, 627, 1339, 3875, 628, 629, 3070, 4565, 2118, 4818, 4860, 2557, 2795, 2906, 2103, 4869, 2029, 3299, 3118, 4932, 4838, 4901, 2377, 4657, 4646, 4875, 4627, 4515, 4876, 4965, 4605, 4576, 2028, 4985, 4799, 4850, 1103, 4747, 1614, 1947, 2237, 630, 631, 4618, 4479, 3766, 632, 1104, 1105, 4886, 1106, 2432, 4827, 633, 1107, 4530, 634, 2371, 4651, 1108, 4536, 635, 1813, 636, 2431, 4866, 4991, 4814, 4560, 4956, 637, 4555, 1109, 4941, 4577, 2213, 638, 639, 3706, 1110, 640, 1111, 641, 4581, 4893, 4831, 2481, 4644, 4734, 2190, 4524, 4881, 4837, 4888, 4648, 642, 4548, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4760, 1115, 4918, 4688, 3920, 644, 4723, 4949, 4748, 4607, 4719, 1116, 645, 646, 4611, 2353, 4645, 4998, 2307, 647, 1801, 4279, 3762, 1637, 1509, 4843, 4797, 4590, 4516, 4995, 4541, 2200, 4580, 4858, 648, 649, 2502, 1117, 4780, 4800, 4551, 4674, 1118, 1119, 2965, 2369, 2695, 3293, 650, 651, 4730, 4862, 4722, 4944, 1120, 652, 1121, 4983, 4784, 4877, 4643, 653, 654, 4909, 1122, 3264, 655, 2076, 4849, 656, 657, 2447, 658, 659, 2191, 4035, 2910, 3391, 2808, 3099, 2913, 3676, 2852, 3284, 2731, 1609, 2375, 2201, 3537, 3994, 1319, 4467, 1802, 1227, 1623, 660, 4844, 2086, 4770, 2192, 1123, 661, 2364, 662, 663, 664, 1124, 1125, 665, 2487, 4899, 2138, 1126, 666, 667, 4569, 4554, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 674, 2456, 4606, 4910, 2123, 1136, 1137, 2358, 5005, 2208, 4952, 4945, 1933, 1731, 1793, 675, 676, 3342, 1138, 1139, 677, 4840, 4597, 4870, 1140, 4990, 4954, 4916, 678, 1141, 4737, 4527, 2253, 4531, 679, 1142, 1143, 680, 4771, 1144, 4638, 681, 4705, 4525, 4522, 4898, 2325, 2415, 5004, 1145, 682, 683, 1146, 684, 685, 2445, 686, 687, 4861, 2157, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4793, 4575, 2160, 4828, 4731, 4718, 4802, 4750, 1150, 2081, 2258, 697, 1795, 1372, 1695, 1866, 1151, 4115, 1719, 2280, 4662, 698, 2176, 2194, 2217, 1920, 699, 700, 701, 4999, 1544, 4739, 2512, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 4824, 705, 2340, 4182, 706, 4652, 2442, 2484, 707, 708, 709, 4667, 1153, 2412, 4476, 2053, 710, 1375, 711, 712, 713, 4829, 4738, 4811, 4570, 4961, 1245, 714, 2045, 4707, 715, 2169, 4713, 4879, 1841, 4234, 4924, 1154, 1314, 4096, 2023, 716, 717, 4895, 2414, 718, 1155, 719, 720, 2063, 1311, 4921, 721, 4642, 722, 4885, 4666, 2249, 4217, 1156, 723, 1157, 1627, 2116, 4566, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 730, 731, 2024, 1846, 2507, 2368, 2019, 2333, 3815, 2005, 2266, 1964, 2347, 2385, 732, 2177, 2059, 4958, 1822, 4815, 1262, 2094, 4480, 1537, 1291, 2419, 1318, 2283, 4976, 4294, 2486, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4679, 4835, 2259, 4839, 4571, 2275, 1166, 4968, 2250, 4912, 2423, 737, 1167, 4989, 4684, 4669, 4552, 4612, 2127, 1745, 1973, 4767, 1168, 2273, 2310, 3807, 4307, 1169, 2303, 1454, or 1781.

A VP capsid polypeptide may have a 581 to 589 region having a sequence of SEQ ID NO: 5014, wherein X⁶ of SEQ ID NO: 5014 is not E. In some embodiments, the 581 to 589 region has a sequence of any one of SEQ ID NOs: 2498, 4884, 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 2469, 4889, 21, 3192, 4942, 22, 23, 1638, 748, 24, 25, 749, 26, 27, 1703, 1836, 28, 2038, 2301, 3660, 29, 750, 30, 31, 32, 2095, 1640, 33, 35, 36, 37, 4742, 2450, 38, 4925, 39, 751, 752, 2144, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2080, 50, 1308, 1230, 3960, 1378, 2994, 3080, 2589, 3681, 51, 4694, 2257, 2167, 52, 760, 53, 2389, 4934, 4951, 4755, 761, 762, 4659, 54, 2146, 3925, 55, 3654, 2087, 2346, 4766, 4682, 4984, 4744, 56, 763, 764, 4626, 4702, 58, 59, 60, 61, 4086, 1740, 1660, 765, 766, 62, 1966, 2032, 2499, 2332, 767, 63, 2096, 2181, 4913, 2438, 2041, 64, 768, 769, 770, 2384, 2402, 771, 65, 66, 2111, 67, 772, 2458, 2197, 68, 3816, 69, 2599, 2706, 2497, 70, 2234, 2183, 2399, 71, 4769, 2366, 72, 73, 74, 3865, 1549, 1361, 2453, 3882, 773, 3713, 1942, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2132, 2379, 82, 4065, 83, 84, 2030, 1294, 2413, 2064, 1484, 2299, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 93, 2356, 784, 785, 786, 94, 95, 787, 788, 1831, 1867, 96, 4458, 97, 1970, 4656, 2173, 1936, 2349, 1590, 4585, 98, 99, 100, 789, 790, 1298, 101, 1688, 791, 792, 2007, 2070, 102, 793, 4632, 103, 794, 795, 104, 105, 106, 2229, 796, 4980, 4691, 107, 2440, 1849, 108, 2014, 797, 4736, 2509, 2018, 2227, 4787, 109, 1579, 110, 1906, 111, 2428, 798, 112, 2411, 113, 2359, 799, 4848, 800, 2461, 1444, 2313, 2350, 4579, 4821, 801, 114, 2329, 4864, 4834, 115, 1344, 116, 802, 117, 118, 119, 4929, 4677, 120, 4791, 2158, 121, 803, 804, 1463, 805, 122, 2139, 2290, 1760, 2465, 2424, 2058, 2230, 124, 4717, 806, 125, 126, 2370, 127, 4521, 128, 4971, 4994, 807, 2085, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 4544, 144, 145, 816, 4654, 2125, 4663, 4810, 4816, 1634, 1302, 817, 146, 818, 147, 148, 149, 4546, 150, 151, 152, 153, 154, 155, 819, 820, 156, 2149, 157, 2295, 821, 158, 159, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 2170, 4809, 4520, 180, 826, 827, 181, 828, 4695, 2130, 4911, 182, 4582, 4873, 4596, 2020, 2189, 183, 4641, 829, 184, 185, 2244, 4647, 186, 187, 188, 189, 190, 2114, 830, 191, 192, 193, 194, 2071, 4615, 2361, 195, 4852, 2187, 4914, 4947, 196, 1391, 197, 4534, 4726, 4788, 198, 199, 4920, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2090, 2241, 217, 218, 4653, 219, 1612, 220, 221, 222, 839, 223, 4953, 4974, 4790, 4915, 2464, 4567, 4774, 4964, 2360, 840, 224, 4928, 4741, 225, 2088, 4751, 5007, 4542, 226, 227, 4890, 4519, 4883, 228, 4786, 229, 230, 231, 2393, 2448, 841, 842, 232, 233, 4782, 234, 4962, 2269, 843, 235, 844, 236, 237, 4930, 845, 846, 238, 847, 4704, 239, 848, 849, 2150, 850, 851, 852, 853, 1536, 240, 854, 241, 242, 243, 244, 245, 246, 247, 248, 4923, 2245, 4696, 855, 4634, 856, 249, 250, 251, 252, 253, 254, 255, 857, 2115, 4880, 256, 257, 258, 259, 858, 859, 260, 261, 860, 861, 862, 262, 4966, 863, 864, 263, 4749, 4970, 2296, 2318, 4535, 865, 2326, 264, 1241, 265, 266, 866, 1477, 267, 867, 4977, 268, 868, 269, 4668, 5002, 270, 869, 870, 271, 871, 872, 272, 4822, 4709, 873, 4655, 4972, 2392, 2317, 874, 1252, 273, 1591, 4975, 875, 274, 2025, 275, 1980, 276, 876, 877, 2066, 277, 278, 279, 4670, 280, 2323, 2288, 2334, 4689, 281, 878, 2416, 1892, 879, 1539, 2214, 2215, 880, 2327, 881, 882, 4601, 883, 1295, 3601, 3877, 1930, 1420, 1678, 1460, 2306, 884, 282, 1799, 283, 284, 4988, 4871, 4553, 4772, 4817, 2260, 285, 1626, 2082, 885, 286, 1374, 886, 2119, 2504, 2468, 287, 1620, 1290, 2425, 2482, 4671, 2282, 288, 2354, 1354, 2033, 289, 887, 290, 888, 291, 1956, 2467, 889, 2357, 4547, 4765, 292, 4794, 2391, 4324, 890, 294, 295, 4138, 891, 2297, 296, 2224, 3903, 297, 892, 3829, 298, 299, 3809, 4271, 3844, 1436, 300, 1515, 1617, 4701, 2174, 301, 1961, 4649, 5010, 302, 2060, 303, 304, 305, 2452, 2147, 4574, 4538, 306, 2293, 1589, 893, 4946, 4370, 4841, 2342, 307, 308, 309, 894, 2246, 2155, 3646, 4059, 1431, 1331, 310, 311, 4836, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 904, 1635, 4820, 905, 2506, 331, 4706, 2092, 4484, 1756, 906, 907, 4661, 2179, 4584, 332, 4907, 4635, 4529, 4683, 4523, 4561, 333, 2337, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4783, 4319, 3906, 1533, 1755, 1888, 4248, 1557, 2153, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 2444, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 908, 1935, 4711, 4728, 2460, 4517, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 4943, 3002, 2986, 910, 2979, 2542, 2331, 2492, 1762, 4472, 1472, 1529, 2319, 2131, 4057, 2048, 4887, 1976, 4507, 1583, 338, 2078, 2372, 2102, 339, 911, 1711, 340, 912, 2013, 913, 2277, 914, 341, 915, 4672, 4902, 916, 342, 1594, 4440, 917, 343, 344, 4559, 918, 2233, 2439, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 2471, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 375, 2343, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 2383, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2129, 2345, 4471, 952, 953, 401, 402, 2475, 403, 404, 4758, 955, 2437, 4139, 1482, 3602, 4434, 3721, 1592, 1825, 1698, 1519, 1352, 4202, 4409, 1977, 1917, 405, 406, 407, 408, 956, 2272, 957, 2267, 1631, 1684, 409, 2054, 3889, 958, 410, 2117, 411, 959, 412, 960, 2443, 4712, 4619, 2285, 413, 414, 415, 961, 2352, 2417, 962, 1905, 416, 963, 417, 418, 2382, 3675, 1833, 3974, 419, 964, 421, 965, 966, 967, 4919, 968, 2365, 969, 970, 422, 1897, 2056, 2309, 4733, 1272, 4754, 2207, 971, 423, 4133, 3830, 4865, 972, 424, 973, 425, 2068, 4955, 426, 974, 2109, 2380, 427, 1708, 4676, 428, 4141, 429, 430, 3749, 431, 1619, 975, 4044, 3590, 1944, 4126, 1923, 1706, 432, 976, 977, 978, 4867, 4400, 433, 434, 1606, 2143, 979, 435, 980, 2604, 2895, 981, 436, 3448, 4959, 2188, 437, 438, 2055, 1692, 982, 2388, 2433, 2243, 983, 2035, 2120, 4940, 439, 440, 2474, 442, 4900, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 453, 992, 1728, 993, 4703, 994, 995, 454, 1696, 996, 2039, 455, 997, 4874, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 466, 2328, 467, 468, 469, 1231, 1468, 470, 471, 2496, 472, 473, 1005, 474, 475, 2108, 4725, 476, 1273, 1007, 1744, 1488, 1659, 477, 478, 4598, 1604, 4753, 2493, 479, 1008, 1009, 480, 1011, 1012, 1013, 1014, 481, 4313, 482, 483, 1499, 484, 1015, 4761, 2091, 2386, 2513, 1016, 4073, 1777, 2172, 485, 2205, 4592, 4545, 486, 1458, 4274, 2122, 4905, 5003, 4599, 4796, 2152, 4539, 4528, 4904, 4842, 4986, 4846, 4650, 4595, 2195, 4681, 2142, 487, 4710, 1775, 4205, 1526, 4586, 4602, 4518, 1697, 1017, 2000, 4692, 4795, 2480, 2017, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 2202, 5001, 2136, 493, 2211, 4532, 494, 2421, 495, 4090, 2378, 2104, 2210, 2247, 2478, 2047, 496, 4620, 4978, 4614, 1019, 2012, 1803, 3642, 497, 1322, 4752, 1020, 498, 1021, 2235, 2302, 2488, 4665, 4540, 5006, 4699, 4608, 4781, 499, 2049, 4685, 500, 501, 1022, 502, 1333, 503, 1023, 4686, 4804, 504, 4727, 505, 506, 4938, 4805, 2065, 1890, 4891, 1024, 507, 508, 509, 2110, 1025, 510, 1026, 3608, 1732, 511, 2397, 1864, 1816, 1531, 512, 513, 2451, 3636, 5012, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 2093, 4746, 2489, 4922, 2145, 1030, 4832, 4950, 1261, 4027, 3478, 3763, 2959, 1293, 2462, 517, 3197, 1595, 3413, 2238, 2067, 1031, 2228, 518, 4698, 4616, 2057, 2305, 2133, 2061, 4526, 4617, 1032, 1033, 2429, 2311, 1675, 4785, 1797, 519, 4578, 2271, 4693, 4604, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 535, 1038, 536, 1039, 537, 538, 539, 1040, 3767, 1929, 1042, 3683, 2100, 4853, 2300, 2165, 540, 2105, 541, 1043, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 1044, 3720, 4351, 4414, 1673, 3453, 3254, 2501, 1649, 543, 544, 545, 546, 1045, 547, 4240, 3580, 4680, 1046, 548, 2062, 549, 1047, 550, 2598, 551, 552, 553, 3819, 1259, 4062, 1048, 554, 2841, 1049, 1050, 3515, 2219, 1433, 1323, 4301, 555, 556, 1051, 557, 558, 1052, 1053, 3738, 1567, 559, 1054, 1270, 3857, 1055, 2151, 560, 2175, 2089, 1056, 4514, 2198, 2193, 1257, 1238, 3662, 561, 2186, 1057, 4637, 562, 1058, 563, 4872, 2097, 2436, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2185, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2255, 2113, 590, 591, 1085, 592, 2308, 593, 594, 2274, 595, 4931, 2407, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 1086, 601, 4017, 4477, 1794, 2483, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 2320, 2069, 610, 611, 1093, 612, 1094, 3701, 4389, 613, 4897, 1603, 614, 1095, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4801, 4855, 4847, 2398, 2239, 1101, 1667, 1384, 623, 4745, 3700, 1788, 1600, 624, 2098, 625, 4550, 4721, 4960, 4878, 2473, 4792, 4675, 1102, 1423, 626, 2771, 627, 1339, 3875, 628, 629, 3070, 4565, 2118, 2015, 4818, 2557, 2795, 2906, 2103, 4869, 2029, 3299, 3118, 4932, 4838, 4901, 4908, 4996, 4779, 4657, 4646, 4875, 4627, 4515, 4876, 4965, 4605, 4576, 2028, 4985, 4850, 1103, 4757, 4747, 1614, 1947, 2237, 630, 631, 4997, 4618, 4479, 3766, 632, 1104, 1105, 4886, 1106, 2432, 633, 1107, 4530, 634, 2371, 4651, 1108, 4536, 635, 1813, 636, 2431, 4866, 4991, 4814, 4560, 4956, 637, 4555, 1109, 4941, 4577, 4633, 2213, 638, 639, 3706, 1110, 640, 1111, 641, 4581, 4893, 4831, 2481, 4644, 4734, 2190, 4524, 4881, 4837, 4888, 4648, 642, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4760, 4629, 1115, 4918, 4688, 3920, 644, 4723, 4949, 4748, 4607, 4719, 1116, 645, 646, 4611, 2353, 4645, 4603, 4762, 4819, 2221, 4636, 4992, 4998, 2307, 647, 1801, 4279, 3762, 1637, 1509, 4843, 4797, 4590, 4995, 4541, 2200, 4580, 4858, 648, 649, 2502, 1117, 4780, 4800, 2477, 4724, 4551, 4674, 1118, 1119, 2965, 2369, 2695, 3293, 650, 651, 4730, 4862, 4722, 4944, 1120, 652, 1121, 4983, 4784, 4877, 4643, 653, 654, 4909, 1122, 3264, 655, 2076, 4849, 656, 657, 2447, 658, 659, 4035, 2910, 3391, 2808, 3099, 2913, 3676, 2852, 3284, 2731, 1609, 2375, 2201, 3537, 3994, 1319, 4467, 1802, 1227, 1623, 660, 4844, 2086, 4770, 4714, 4979, 4812, 2192, 1123, 661, 2364, 662, 663, 664, 1124, 1125, 665, 2487, 4899, 2138, 1126, 666, 667, 4569, 4554, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 2456, 4606, 4588, 4963, 4851, 4558, 4557, 4697, 4610, 4773, 4687, 4708, 4910, 2123, 1136, 1137, 2358, 5005, 2208, 4952, 4945, 1933, 1731, 1793, 675, 676, 3342, 1138, 677, 4597, 4870, 1140, 4990, 4954, 4916, 678, 1141, 4737, 4527, 2253, 4531, 679, 1142, 1143, 680, 4771, 1144, 4638, 681, 4705, 4525, 4522, 4898, 2325, 2415, 5004, 1145, 682, 683, 2141, 1146, 684, 685, 2445, 686, 687, 4861, 2157, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4690, 4859, 4793, 4575, 2160, 4828, 4731, 4718, 4802, 4750, 1150, 2081, 2258, 697, 1795, 1372, 1695, 1866, 1151, 4115, 1719, 2280, 4662, 698, 2176, 2194, 2217, 1920, 699, 700, 701, 4999, 1544, 4739, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 4824, 705, 2340, 4182, 706, 2442, 2484, 707, 708, 709, 4667, 1153, 2412, 4732, 4476, 2053, 710, 1375, 711, 712, 713, 4829, 4738, 4811, 4570, 4961, 1245, 714, 2045, 4707, 715, 2169, 4713, 1841, 4234, 4924, 1154, 1314, 4096, 2023, 716, 717, 4895, 718, 1155, 719, 720, 2063, 1311, 721, 4642, 722, 4833, 4903, 4885, 4666, 2249, 4217, 1156, 723, 1157, 1627, 2116, 4566, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 2248, 730, 731, 2024, 1846, 2507, 2368, 1162, 2019, 2333, 3815, 2005, 2266, 1964, 2347, 2385, 732, 2177, 2059, 4958, 4927, 1822, 4815, 1262, 2094, 4480, 1537, 1291, 2419, 1318, 2283, 4976, 4628, 4294, 2486, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4679, 4835, 2259, 4839, 4571, 1166, 4968, 2250, 4912, 2423, 737, 1167, 4989, 4684, 4669, 4552, 4612, 2127, 1745, 1973, 4767, 1168, 2273, 2310, 3807, 4307, 1169, 2303, 1454, or 1781.

A VP capsid polypeptide may have a 581 to 589 region having a sequence of SEQ ID NO: 5014, wherein X³ of SEQ ID NO: 5014 is not E and X⁶ of SEQ ID NO: 5014 is not E. In some embodiments, the 581 to 589 region has a sequence of any one of SEQ ID NOs: 2498, 4884, 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 4889, 21, 3192, 4942, 22, 23, 1638, 748, 24, 25, 749, 26, 27, 1703, 1836, 28, 2038, 2301, 3660, 29, 750, 30, 31, 32, 2095, 1640, 33, 35, 36, 37, 4742, 2450, 38, 4925, 39, 751, 752, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2080, 50, 1308, 1230, 3960, 1378, 2994, 3080, 2589, 3681, 51, 4694, 2257, 52, 760, 53, 2389, 4934, 4951, 761, 762, 4659, 54, 2146, 3925, 55, 3654, 2087, 2346, 4766, 4682, 4984, 4744, 56, 763, 764, 4626, 4702, 58, 59, 60, 61, 4086, 1740, 1660, 765, 766, 62, 1966, 2032, 2499, 2332, 767, 63, 2096, 2181, 4913, 64, 768, 769, 770, 2384, 2402, 771, 65, 66, 2111, 67, 772, 2458, 2197, 68, 3816, 69, 2599, 2706, 2497, 70, 2234, 2183, 2399, 71, 4769, 2366, 72, 73, 74, 3865, 1549, 1361, 2453, 3882, 773, 3713, 1942, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2132, 2379, 82, 4065, 83, 84, 2030, 1294, 2413, 2064, 1484, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 93, 2356, 784, 785, 786, 94, 95, 787, 788, 1831, 1867, 96, 4458, 97, 1970, 4656, 2173, 1936, 2349, 1590, 4585, 98, 99, 100, 789, 790, 1298, 101, 1688, 791, 792, 2007, 2070, 102, 793, 4632, 103, 794, 795, 104, 105, 106, 2229, 796, 4980, 4691, 107, 2440, 1849, 108, 2014, 797, 4736, 2509, 2018, 2227, 4787, 109, 1579, 110, 1906, 111, 2428, 798, 112, 2411, 113, 2359, 799, 4848, 800, 2461, 1444, 2313, 2350, 4579, 4821, 801, 114, 2329, 4834, 115, 1344, 116, 802, 117, 118, 119, 4929, 4677, 120, 2158, 121, 803, 804, 1463, 805, 122, 2139, 2290, 1760, 2465, 2424, 2058, 2230, 124, 4717, 806, 125, 126, 2370, 127, 4521, 128, 4971, 4994, 807, 2085, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 4544, 144, 145, 816, 4654, 4816, 1634, 1302, 817, 146, 818, 147, 148, 149, 4546, 150, 151, 152, 153, 154, 155, 819, 820, 156, 2149, 157, 2295, 821, 158, 159, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 2170, 4809, 4520, 180, 826, 827, 181, 828, 4695, 4911, 182, 4582, 4873, 4596, 2020, 183, 4641, 829, 184, 185, 2244, 4647, 186, 187, 188, 189, 190, 2114, 830, 191, 192, 193, 194, 2071, 4615, 2361, 195, 4852, 2187, 4914, 4947, 196, 1391, 197, 4534, 4726, 4788, 198, 199, 4920, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2241, 217, 218, 4653, 219, 1612, 220, 221, 222, 839, 223, 4953, 2360, 840, 224, 4928, 4741, 225, 2088, 4751, 5007, 4542, 226, 227, 4890, 4519, 4883, 228, 4786, 229, 230, 231, 2393, 2448, 841, 842, 232, 233, 4782, 234, 4962, 2269, 843, 235, 844, 236, 237, 4930, 845, 846, 238, 847, 4704, 239, 848, 849, 2150, 850, 851, 852, 853, 1536, 240, 854, 241, 242, 243, 244, 245, 246, 247, 248, 4923, 2245, 4696, 855, 4634, 856, 249, 250, 251, 252, 253, 254, 255, 857, 2115, 4880, 256, 257, 258, 259, 858, 859, 260, 261, 860, 861, 862, 262, 4966, 863, 864, 263, 4749, 4970, 2296, 2318, 865, 2326, 264, 1241, 265, 266, 866, 1477, 267, 867, 4977, 268, 868, 269, 4668, 5002, 270, 869, 870, 271, 871, 872, 272, 4822, 4709, 873, 4655, 4972, 2392, 874, 1252, 273, 1591, 4975, 875, 274, 2025, 275, 1980, 276, 876, 877, 2066, 277, 278, 279, 4670, 280, 2323, 2288, 2334, 4689, 281, 878, 1892, 879, 1539, 2214, 2215, 880, 2327, 881, 882, 4601, 883, 1295, 3601, 3877, 1930, 1420, 1678, 1460, 2306, 884, 282, 1799, 283, 284, 4988, 4553, 4772, 4817, 2260, 285, 1626, 2082, 885, 286, 1374, 886, 2119, 2468, 287, 1620, 1290, 2425, 2482, 4671, 2282, 288, 2354, 1354, 2033, 289, 887, 290, 888, 291, 1956, 2467, 889, 2357, 4547, 4765, 292, 4794, 2391, 4324, 890, 294, 295, 4138, 891, 2297, 296, 2224, 3903, 297, 892, 3829, 298, 299, 3809, 4271, 3844, 1436, 300, 1515, 1617, 4701, 2174, 301, 1961, 4649, 5010, 302, 303, 304, 305, 2452, 2147, 306, 2293, 1589, 893, 4370, 4841, 2342, 307, 308, 309, 894, 2246, 2155, 3646, 4059, 1431, 1331, 310, 311, 4836, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 904, 1635, 4820, 905, 2506, 331, 4706, 2092, 4484, 1756, 906, 907, 4661, 2179, 4584, 332, 4907, 4635, 4529, 4683, 4523, 4561, 333, 2337, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4783, 4319, 3906, 1533, 1755, 1888, 4248, 1557, 2153, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 2444, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 908, 1935, 4711, 4728, 2460, 4517, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 910, 2979, 2542, 2492, 1762, 4472, 1472, 1529, 2319, 2131, 4057, 2048, 1976, 4507, 1583, 338, 2078, 2372, 339, 911, 1711, 340, 912, 2013, 913, 2277, 914, 341, 915, 4672, 4902, 916, 342, 1594, 4440, 917, 343, 344, 4559, 918, 2233, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 2471, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 375, 2343, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 2383, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2129, 2345, 4471, 952, 953, 402, 2475, 403, 404, 4758, 955, 2437, 4139, 1482, 3602, 4434, 3721, 1592, 1825, 1698, 1519, 1352, 4202, 4409, 1977, 1917, 405, 406, 407, 408, 956, 2272, 957, 2267, 1631, 1684, 409, 2054, 3889, 958, 410, 2117, 411, 959, 412, 960, 2443, 4712, 4619, 2285, 414, 415, 961, 2352, 2417, 962, 1905, 416, 963, 417, 418, 3675, 1833, 3974, 419, 964, 421, 965, 966, 967, 968, 2365, 969, 970, 422, 1897, 2056, 2309, 4733, 1272, 4754, 2207, 971, 423, 4133, 3830, 4865, 972, 424, 973, 425, 2068, 4955, 426, 974, 427, 1708, 4676, 428, 4141, 430, 3749, 431, 1619, 975, 4044, 3590, 1944, 4126, 1923, 1706, 432, 976, 977, 978, 4867, 4400, 433, 434, 1606, 2143, 979, 435, 980, 2604, 2895, 981, 436, 3448, 4959, 2188, 437, 438, 1692, 982, 2388, 2433, 2243, 983, 2035, 2120, 4940, 439, 440, 2474, 442, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 453, 992, 1728, 993, 4703, 994, 995, 454, 1696, 996, 2039, 455, 997, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 467, 468, 469, 1231, 1468, 470, 471, 2496, 472, 473, 1005, 474, 475, 2108, 4725, 476, 1273, 1007, 1744, 1488, 1659, 477, 478, 4598, 1604, 4753, 2493, 479, 1008, 1009, 480, 1011, 1012, 1013, 1014, 481, 4313, 482, 483, 1499, 484, 1015, 4761, 2091, 2513, 1016, 4073, 1777, 2172, 485, 2205, 4592, 4545, 486, 1458, 4274, 2122, 4905, 5003, 4599, 4796, 2152, 4539, 4528, 4904, 4842, 4986, 4846, 4650, 4595, 2195, 4681, 2142, 487, 4710, 1775, 4205, 1526, 4586, 4602, 4518, 1697, 1017, 2000, 4692, 4795, 2480, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 2202, 5001, 2136, 493, 2211, 4532, 494, 2421, 495, 4090, 2378, 2104, 2210, 2247, 2478, 2047, 496, 4620, 4978, 4614, 1019, 2012, 1803, 3642, 497, 1322, 1020, 498, 1021, 2235, 2302, 2488, 4665, 4540, 5006, 499, 2049, 4685, 500, 501, 1022, 502, 1333, 503, 1023, 4686, 4804, 504, 4727, 505, 506, 4938, 4805, 2065, 1890, 4891, 1024, 507, 508, 509, 1025, 510, 1026, 3608, 1732, 511, 2397, 1864, 1816, 1531, 512, 513, 2451, 3636, 5012, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 2093, 4746, 2489, 4922, 2145, 1030, 4832, 4950, 1261, 4027, 3478, 3763, 2959, 1293, 2462, 517, 3197, 1595, 3413, 2238, 1031, 2228, 518, 2057, 2305, 2133, 2061, 4526, 4617, 1033, 2429, 2311, 1675, 4785, 1797, 519, 4578, 2271, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 535, 1038, 536, 1039, 537, 538, 539, 1040, 3767, 1929, 1042, 3683, 2100, 4853, 2300, 2165, 540, 541, 1043, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 1044, 3720, 4351, 4414, 1673, 3453, 3254, 2501, 1649, 543, 544, 545, 546, 1045, 547, 4240, 3580, 4680, 1046, 548, 2062, 1047, 550, 2598, 551, 552, 553, 3819, 1259, 4062, 1048, 554, 2841, 1049, 1050, 3515, 2219, 1433, 1323, 4301, 555, 556, 1051, 557, 558, 1052, 1053, 3738, 1567, 559, 1054, 1270, 3857, 1055, 2151, 560, 2175, 2089, 1056, 2198, 2193, 1257, 1238, 3662, 561, 2186, 1057, 4637, 562, 1058, 563, 4872, 2097, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2255, 2113, 590, 591, 1085, 592, 2308, 593, 594, 2274, 595, 4931, 2407, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 1086, 601, 4017, 4477, 1794, 2483, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 610, 611, 1093, 612, 1094, 3701, 4389, 613, 4897, 1603, 614, 1095, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4801, 2398, 2239, 1101, 1667, 1384, 623, 4745, 3700, 1788, 1600, 624, 2098, 625, 4550, 4721, 4960, 4878, 2473, 4792, 4675, 1102, 1423, 626, 2771, 627, 1339, 3875, 628, 629, 3070, 4565, 2118, 4818, 2557, 2795, 2906, 2103, 4869, 2029, 3299, 3118, 4932, 4838, 4901, 4657, 4646, 4875, 4627, 4515, 4876, 4965, 4605, 4576, 2028, 4985, 4850, 1103, 4747, 1614, 1947, 2237, 630, 631, 4618, 4479, 3766, 632, 1104, 1105, 4886, 1106, 2432, 633, 1107, 4530, 634, 2371, 4651, 1108, 4536, 635, 1813, 636, 2431, 4866, 4991, 4814, 4560, 4956, 637, 4555, 1109, 4941, 4577, 2213, 638, 639, 3706, 1110, 640, 1111, 641, 4581, 4893, 4831, 2481, 4644, 4734, 2190, 4524, 4881, 4837, 4888, 4648, 642, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4760, 1115, 4918, 4688, 3920, 644, 4723, 4949, 4748, 4607, 4719, 1116, 645, 646, 4611, 2353, 4645, 4998, 2307, 647, 1801, 4279, 3762, 1637, 1509, 4843, 4797, 4590, 4995, 4541, 2200, 4580, 4858, 648, 649, 2502, 1117, 4780, 4800, 4551, 4674, 1118, 1119, 2965, 2369, 2695, 3293, 650, 651, 4730, 4862, 4722, 4944, 1120, 652, 1121, 4983, 4784, 4877, 4643, 653, 654, 4909, 1122, 3264, 655, 2076, 4849, 656, 657, 2447, 658, 659, 4035, 2910, 3391, 2808, 3099, 2913, 3676, 2852, 3284, 2731, 1609, 2375, 2201, 3537, 3994, 1319, 4467, 1802, 1227, 1623, 660, 4844, 2086, 4770, 2192, 1123, 661, 2364, 662, 663, 664, 1124, 1125, 665, 2487, 4899, 2138, 1126, 666, 667, 4569, 4554, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 2456, 4606, 4910, 2123, 1136, 1137, 2358, 5005, 2208, 4952, 4945, 1933, 1731, 1793, 675, 676, 3342, 1138, 677, 4597, 4870, 1140, 4990, 4954, 4916, 678, 1141, 4737, 4527, 2253, 4531, 679, 1142, 1143, 680, 4771, 1144, 4638, 681, 4705, 4525, 4522, 4898, 2325, 2415, 5004, 1145, 682, 683, 1146, 684, 685, 2445, 686, 687, 4861, 2157, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4793, 4575, 2160, 4828, 4731, 4718, 4802, 4750, 1150, 2081, 2258, 697, 1795, 1372, 1695, 1866, 1151, 4115, 1719, 2280, 4662, 698, 2176, 2194, 2217, 1920, 699, 700, 701, 4999, 1544, 4739, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 4824, 705, 2340, 4182, 706, 2442, 2484, 707, 708, 709, 4667, 1153, 2412, 4476, 2053, 710, 1375, 711, 712, 713, 4829, 4738, 4811, 4570, 4961, 1245, 714, 2045, 4707, 715, 2169, 4713, 1841, 4234, 4924, 1154, 1314, 4096, 2023, 716, 717, 4895, 718, 1155, 719, 720, 2063, 1311, 721, 4642, 722, 4885, 4666, 2249, 4217, 1156, 723, 1157, 1627, 2116, 4566, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 730, 731, 2024, 1846, 2507, 2368, 2019, 2333, 3815, 2005, 2266, 1964, 2347, 2385, 732, 2177, 2059, 4958, 1822, 4815, 1262, 2094, 4480, 1537, 1291, 2419, 1318, 2283, 4976, 4294, 2486, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4679, 4835, 2259, 4839, 4571, 1166, 4968, 2250, 4912, 2423, 737, 1167, 4989, 4684, 4669, 4552, 4612, 2127, 1745, 1973, 4767, 1168, 2273, 2310, 3807, 4307, 1169, 2303, 1454, or 1781.

RPE Tissue-Tropic AAV VP Capsid Polypeptides. Described herein are engineered AAV VP capsid polypeptides comprising a variant 581 to 589 region predicted to confer increased RPE tissue tropism compared to a wild type AAV VP capsid polypeptide (e.g., a wild type AAV5 capsid polypeptide of SEQ ID NO: 1), generated and selected using machine learning. In some embodiments, an RPE tissue-tropic engineered AAV VP capsid polypeptide may also have tropism for choroid tissue. TABLE 9 provides 581 to 589 region sequences predicted to confer RPE tissue tropism or preference.

TABLE 9
Additional Exemplary 581 to 589
Region Sequences for RPE Targeting

| SEQ ID | |
|---|---|
| NO | 581 to 589 Region |
| 4514 | QTEFVCSGK |
| 4515 | TEINDTKST |
| 4516 | TNLPMEMIP |
| 4517 | HPDPTAEYA |
| 4518 | NHDVTPREY |
| 4519 | EPTACGVEI |
| 4520 | EAVQTAEHN |
| 4521 | DPNQCWMMN |
| 4522 | TWLELDMEC |
| 4523 | HATVFDENN |
| 4524 | TIPMHDQAK |
| 4525 | TWLDKHCVA |
| 4526 | PPTVTQCMH |
| 4527 | TVPCNGEHS |
| 4528 | NEHTDVQGE |
| 4529 | HATMCNQKS |
| 4530 | TGTVMQKAD |
| 4531 | TVPMKGMEE |
| 4532 | NMTHTQNEI |
| 4533 | DQTLCEEPP |
| 4534 | EHTIFGESG |
| 4535 | EVENKIYGH |
| 4536 | THGSVCRPD |
| 4537 | VATPECQAY |
| 4538 | GNEPDVQYN |
| 4539 | NEHSTGKAC |
| 4540 | NTDWLCDMN |
| 4541 | TNMMCNEGP |
| 4542 | EPDYKVAQP |
| 4543 | PETVGYKHC |
| 4544 | DTQFLDMVK |
| 4545 | NATMYVEER |
| 4546 | DVNPRGEAI |
| 4547 | GDNVTVEAP |
| 4548 | TITCQEARC |
| 4549 | SSHNLCCVT |
| 4550 | TATAHCMEG |
| 4551 | TPFADLCVC |
| 4552 | YTDSANNAI |
| 4553 | FQMQMHAYA |
| 4554 | TTDFAGMVQ |
| 4555 | THTCNWAEQ |
| 4556 | DALNREEAC |
| 4557 | TVENKSADA |
| 4558 | TVEMWNYCC |
| 4559 | IEPATPHCD |
| 4560 | THQNTFLTV |
| 4561 | HAVNTCEEQ |
| 4562 | MQNPFYEQT |
| 4563 | MEQKNETSG |
| 4564 | FVTDKEAVA |
| 4565 | TCTDIFHQA |
| 4566 | VTLMKDSAC |
| 4567 | ENEPGQTAC |
| 4568 | VYHCPSEED |
| 4569 | TTCWINNEG |
| 4570 | VQCVCLRNY |
| 4571 | YQDTFHEAN |
| 4572 | PEQTKACYA |
| 4573 | QATPCCEQP |
| 4574 | GNEACPKQI |
| 4575 | VEHTVCEIC |
| 4576 | TEQFAKNSA |
| 4577 | TIDVTHAAC |
| 4578 | PSTVNHCFT |
| 4579 | DHTPGGEPP |
| 4580 | TNTPASHDA |
| 4581 | TILMKHASQ |
| 4582 | EESQTVLIY |
| 4583 | SEIASHKLT |
| 4584 | HAQMQHCYV |
| 4585 | DADTWVMMC |
| 4586 | NGTSAPCML |
| 4587 | YICPSHNLN |
| 4588 | TVEIWNHDT |
| 4589 | MILTHTDVK |
| 4590 | TNLPDHMSY |
| 4591 | ATGQKEMIC |
| 4592 | NATFKNEMP |
| 4593 | NYQFCNEAA |
| 4594 | CQTVHYCPI |
| 4595 | NEVIAYCRH |
| 4596 | EFDPPNRYH |
| 4597 | TVLEMWMMC |
| 4598 | MVQATHCSI |
| 4599 | NEDVYHSEC |
| 4600 | TTVNLNREP |
| 4601 | FIPATNAGS |
| 4602 | NHDTCNEGW |
| 4603 | TNEDQHCAI |
| 4604 | PTEQTNAEA |
| 4605 | TEPGDHCEY |
| 4606 | TVDQMYAIA |
| 4607 | TMQSVNKGA |
| 4608 | NTENEAHAQ |
| 4609 | HAQNREACN |
| 4610 | TVEPTFAGY |
| 4611 | TMVQHTKSI |
| 4612 | YTFQCNAEA |
| 4613 | EAQFNSEHT |
| 4614 | NQTPGDKDG |
| 4615 | EHFNPVEYT |
| 4616 | PNEQYGNWI |
| 4617 | PPVQVGFAS |
| 4618 | TGHMNCEYY |
| 4619 | LNTVCYDKY |
| 4620 | NQTACYKWD |
| 4621 | VAYQKVEPT |
| 4622 | VTPPTNQYC |
| 4623 | NETDVEMMK |
| 4624 | EATGCNESD |
| 4625 | VATQCSAER |
| 4626 | ATVMTHSAC |
| 4627 | TEHQFAQKS |
| 4628 | YGEETSMTC |
| 4629 | TMEQTHNEY |
| 4630 | IPCPTCYGH |
| 4631 | EACDGVKIH |
| 4632 | DAQCYGLPS |
| 4633 | TIEWMNEMY |
| 4634 | ETHVQYMPD |
| 4635 | HATIRGEHQ |
| 4636 | TNEMGVERY |
| 4637 | QTQAPDCYA |
| 4638 | TWFIYCMEH |
| 4639 | NEQYCEHIT |
| 4640 | LHCCTCKEY |
| 4641 | EFHVCYADN |
| 4642 | VTDAYGYEP |
| 4643 | TQCMWDNEN |
| 4644 | TIMQMHNEG |
| 4645 | TNDPLVMEC |
| 4646 | TEHCWMYTC |
| 4647 | EFQACSKYA |
| 4648 | TIQPFMASD |
| 4649 | GLPQCNEHR |
| 4650 | NETWVVCMK |
| 4651 | THDVRCFFA |
| 4652 | VLTNEEKDC |
| 4653 | ELTFHNEYP |
| 4654 | DTVNMYLRQ |
| 4655 | EVTVWHEHN |
| 4656 | CVTAEHNWI |
| 4657 | TEHAYVKAI |
| 4658 | SETHTCYGV |
| 4659 | ATHLVQMYQ |
| 4660 | PAQAWVQTC |
| 4661 | HAQATFEVC |
| 4662 | VHNAQVEMA |
| 4663 | DVENLCAQA |
| 4664 | PETVNHKHA |
| 4665 | NTCPKANEG |
| 4666 | VTFMMNQCK |
| 4667 | VMTPPNEYC |
| 4668 | EVQIYSEAP |
| 4669 | YTDAFANEH |
| 4670 | FALCCNEPQ |
| 4671 | FVTIYAEAC |
| 4672 | IATPPDEQC |
| 4673 | DTHWLCCEV |
| 4674 | TPGEYHMEC |
| 4675 | TATVECCRW |
| 4676 | LWLFHPFAK |
| 4677 | DITPACLHA |
| 4678 | PETWLGEPC |
| 4679 | YQADCDATP |
| 4680 | QNLDKSGAC |
| 4681 | NEYVTCQGD |
| 4682 | ATQMIWMQH |
| 4683 | HATNYVIES |
| 4684 | YTCESDREA |
| 4685 | NTFNEYAWL |
| 4686 | NTLNKNAYG |
| 4687 | TVESLNCCP |
| 4688 | TMIMWYKGA |
| 4689 | FAQPVYEGQ |
| 4690 | VEEATCFVT |
| 4691 | DATMIMYYQ |
| 4692 | NHTNWCESS |
| 4693 | PTEAITCQA |
| 4694 | ASDFICKEC |
| 4695 | EDQACNHSN |
| 4696 | ETGQNHMCP |
| 4697 | TVENWGMAQ |
| 4698 | PNENQHQYL |
| 4699 | NTEIACQLK |
| 4700 | VTQNACLRN |
| 4701 | GLDAIYQMK |
| 4702 | ATVNDYLRP |
| 4703 | MPCAQHALI |
| 4704 | ESDATCCTD |
| 4705 | TWLCPVEEP |
| 4706 | HACQQVEAA |
| 4707 | VQTADMQAS |
| 4708 | TVEYLVHMP |
| 4709 | EVTCNWAQY |
| 4710 | NFQVTHKQI |
| 4711 | HNTATNDMQ |
| 4712 | LNTMMVQIK |
| 4713 | VQYQENARP |
| 4714 | TSEAPHQYK |
| 4715 | VKPEFHNLY |
| 4716 | TTTVENEMG |
| 4717 | DMQNTNHAI |
| 4718 | VETVTDAQC |
| 4719 | TMQYAWMSP |
| 4720 | NATVFEACN |
| 4721 | TATFHYAWP |
| 4722 | TPPNDKEEN |
| 4723 | TMLNSGEFN |
| 4724 | TPEVMHNTA |
| 4725 | MVHATNADS |
| 4726 | EHTVANEAS |
| 4727 | NTNADVAER |
| 4728 | HNTNWCEEY |
| 4729 | IPVTFNNET |
| 4730 | TPNPTDMEI |
| 4731 | VEQMYNEPP |
| 4732 | VNEFYHQIW |
| 4733 | LVDQKNMMQ |
| 4734 | TINEFHMQS |
| 4735 | TMCFNGEYH |
| 4736 | DEHATQCYA |
| 4737 | TVNWRGEQA |
| 4738 | VPVNPGEHK |
| 4739 | VITQTNHEG |
| 4740 | VTPWCFEAC |
| 4741 | ENTCFYHFD |
| 4742 | AINAYNEEH |
| 4743 | VAQGFANEA |
| 4744 | ATQPTPMAH |
| 4745 | TAQNVTEFD |
| 4746 | PFQQFNEAI |
| 4747 | TFHAFIQYK |
| 4748 | TMQMTDQAH |
| 4749 | ETVRTGSNC |
| 4750 | VEWACTNEQ |
| 4751 | EPCPTHKAG |
| 4752 | NSEFDVARG |
| 4753 | MVTAEWADI |
| 4754 | LVHAFYEQR |
| 4755 | ATEPPGVES |
| 4756 | VECMQHEHY |
| 4757 | TFEMFNEGP |
| 4758 | LITLCYEFH |
| 4759 | MQLPVTMRG |
| 4760 | TMDCKWMQC |
| 4761 | NACTCNNEH |
| 4762 | TNEFMWEKA |
| 4763 | HEAQYNEHI |
| 4764 | MIGVTNHAP |
| 4765 | GEFVTHNLT |
| 4766 | ATQHIYDAA |
| 4767 | YTQYEVACP |
| 4768 | MQTDHMCQK |
| 4769 | CENLGVEFK |
| 4770 | TSDVAWEGH |
| 4771 | TVTAFYEIP |
| 4772 | FQTAPHQYC |
| 4773 | TVEQCSHAG |
| 4774 | ENEPPVTVI |
| 4775 | EIQDKHEAL |
| 4776 | SNQQCEEVI |
| 4777 | SQTIACREN |
| 4778 | MQTPSHKSA |
| 4779 | TEERDGNAC |
| 4780 | TPDMKGYQP |
| 4781 | NTESKGEAC |
| 4782 | EQTVFHRLC |
| 4783 | HIDVQCEPD |
| 4784 | TPVQNHHIC |
| 4785 | PSDALTFAI |
| 4786 | EQGAFCGEP |
| 4787 | DENWGVEYS |
| 4788 | EHVQCDAID |
| 4789 | PETFAGVQA |
| 4790 | ENEMCPMMN |
| 4791 | DKELCNESC |
| 4792 | TATPHQKLA |
| 4793 | VEHCYNEGF |
| 4794 | GETVHACKI |
| 4795 | NHTPTDQIA |
| 4796 | NEFVTHYQT |
| 4797 | TNLNYFHYA |
| 4798 | MQNADMTAI |
| 4799 | TEVINEASH |
| 4800 | TPDNLHEGY |
| 4801 | TADSKVEPN |
| 4802 | VEVQCNEQT |
| 4803 | GVQCNTLMI |
| 4804 | NTLNLVAKD |
| 4805 | NTPQLHNEI |
| 4806 | APTPTCCSA |
| 4807 | TITFCYKSD |
| 4808 | IIPPDVQMK |
| 4809 | EATVINDFY |
| 4810 | DVEPLVATC |
| 4811 | VQCPLYMHP |
| 4812 | TSEQKHEAS |
| 4813 | PVTPYGEEL |
| 4814 | THPMAYKEQ |
| 4815 | YAQPCTEFP |
| 4816 | DVHAKYMAH |
| 4817 | FQTINDKSA |
| 4818 | TDHWEMKPI |
| 4819 | TNEFQNMQY |
| 4820 | GYQPWHECI |
| 4821 | DHTQLVREA |
| 4822 | EVTADNHEN |
| 4823 | TIYPAGEAQ |
| 4824 | VLDNTHNEA |
| 4825 | VATPTCYGN |
| 4826 | VTPNEYLPG |
| 4827 | TGQATEMQN |
| 4828 | VEQCLVAAC |
| 4829 | VPMQCDVDA |
| 4830 | SETVWMKHA |
| 4831 | TILVCCQAY |
| 4832 | PILFHNETP |
| 4833 | VTECPVEQP |
| 4834 | DIIQCVLVT |
| 4835 | YQAPPHATC |
| 4836 | GSDACYKFQ |
| 4837 | TIPQNTAPV |
| 4838 | TDVQGHMCI |
| 4839 | YQCPPDRHA |
| 4840 | TVLCGERIH |
| 4841 | GPTAEACFC |
| 4842 | NEQSCTAHI |
| 4843 | TNLIMCMYV |
| 4844 | TSAPYVEEP |
| 4845 | HMLIYGNYH |
| 4846 | NETDCYASA |
| 4847 | TAEMVVHCA |
| 4848 | DGTPMVRYY |
| 4849 | TQMASYAEH |
| 4850 | TEVTFNLHQ |
| 4851 | TVEMTCMMY |
| 4852 | EHLCNTKAA |
| 4853 | QHQAFHEAC |
| 4854 | PTTVFHDMP |
| 4855 | TAEMNYAIA |
| 4856 | HMTCFNEHA |
| 4857 | HHTPAGYYQ |
| 4858 | TNTPDGEDC |
| 4859 | VEEPTHSVQ |
| 4860 | TDKHGEMII |
| 4861 | VACNWGSLT |
| 4862 | TPPILCEMA |
| 4863 | YMPVNNEQI |
| 4864 | DIEFKCEHY |
| 4865 | LVLVPTDAA |
| 4866 | THLHICNPD |
| 4867 | MAQPGVFYA |
| 4868 | TITFVQEVC |
| 4869 | TDMPANASE |
| 4870 | TVLNCMEEN |
| 4871 | FQEDFYALI |
| 4872 | QTVVEHCPP |
| 4873 | EETAIPADC |
| 4874 | MQEPPDMYY |
| 4875 | TEHFHNMPK |
| 4876 | TELNRGEDI |
| 4877 | TPVWQNEGK |
| 4878 | TATIRDEDY |
| 4879 | VRDSLEART |
| 4880 | ETNACDART |
| 4881 | TIPPANEHA |
| 4882 | SATFPNEYA |
| 4883 | EPVPDYEHG |
| 4884 | AADTVHMQY |
| 4885 | VTFIEHLNQ |
| 4886 | TGLIFAEMG |
| 4887 | HVEFIGSAY |
| 4888 | TIQCPCEAY |
| 4889 | AEGVTHAII |
| 4890 | EPNVTVAAS |
| 4891 | NTVEPDNQA |
| 4892 | NWLVEWKAD |
| 4893 | TILPVYEHP |
| 4894 | VTVMIHAIC |
| 4895 | VSMQMHAKC |
| 4896 | TITFLCEED |
| 4897 | STQKCYEHG |
| 4898 | TWLVCCNKS |
| 4899 | TSTVENMDP |
| 4900 | MIEPCGMLQ |
| 4901 | TECCNGESD |
| 4902 | IATVHCMMK |
| 4903 | VTEMVHAAY |
| 4904 | NEQHTVEMR |
| 4905 | NECFKVEHD |
| 4906 | QILMKCETV |
| 4907 | HATFNCEVQ |
| 4908 | TEEMLVCPP |
| 4909 | TQHALDMAR |
| 4910 | TVFLHAHIA |
| 4911 | EEQGEKATI |
| 4912 | YSAVQDEER |
| 4913 | AWVATCNDQ |
| 4914 | EHNSTYEFD |
| 4915 | ENEMMQNDT |
| 4916 | TVLVHGMEN |
| 4917 | TTFAKNEGC |
| 4918 | TMGVQCMEC |
| 4919 | LTEPLDRAA |
| 4920 | EIDPACRHT |
| 4921 | VTCQNEAAC |
| 4922 | PFTVAYEGN |
| 4923 | ETDAFHAYA |
| 4924 | VRLNFCEFD |
| 4925 | AITDKNEGC |
| 4926 | YHCPFGMQP |
| 4927 | YAEQFTAKI |
| 4928 | ENTALNDCG |
| 4929 | DITNLCESC |
| 4930 | ERLPWVQCD |
| 4931 | SITCPGESY |
| 4932 | TDTVFPMDR |
| 4933 | DRLNFCEHC |
| 4934 | ATDAPCEYG |
| 4935 | TKLYHYMET |
| 4936 | SETCKYAIC |
| 4937 | HTVCCEFEG |
| 4938 | NTPFHDCYG |
| 4939 | FAQITEKHA |
| 4940 | MHTSLVEPC |
| 4941 | TICPFDENG |
| 4942 | AEPQKHEAC |
| 4943 | HSEQNGEPR |
| 4944 | TPQNLNHAH |
| 4945 | TVHTCLEAG |
| 4946 | GPESPVNEN |
| 4947 | EHQFTCEAC |
| 4948 | GSHAWMQVT |
| 4949 | TMQMCFKYA |
| 4950 | PILPCKEHC |
| 4951 | ATDNKNHSE |
| 4952 | TVHLCYADI |
| 4953 | ENDFINYGR |
| 4954 | TVLPNYEHA |
| 4955 | LVTNACVEI |
| 4956 | THQPFCKHA |
| 4957 | PEQWYGSAI |
| 4958 | WVTMHQCCK |
| 4959 | MEQATWECC |
| 4960 | TATFQCCYQ |
| 4961 | VQDAIYCYA |
| 4962 | ERDACLEIT |
| 4963 | TVEMFAKHY |
| 4964 | ENETHYKDI |
| 4965 | TENPFHEQP |
| 4966 | ETSKPHYGC |
| 4967 | IVTNKNECC |
| 4968 | YQTWPGRDS |
| 4969 | NIVLCNEHT |
| 4970 | ETVSTHAIY |
| 4971 | DQCATQNAI |
| 4972 | EVVQNHMIP |
| 4973 | QAVHCKEAC |
| 4974 | ENEFTCCYK |
| 4975 | EWLNNSAHA |
| 4976 | YGDAYHAST |
| 4977 | EVLACDKEG |
| 4978 | NQTDFAQQC |
| 4979 | TSENMIESP |
| 4980 | DATFQDCYA |
| 4981 | MQYQVHAAP |
| 4982 | MQTMCNDKY |
| 4983 | TPVIFNEVG |
| 4984 | ATQNIMAGN |
| 4985 | TEQMCSKYA |
| 4986 | NEQWCYKHP |
| 4987 | NYTVIHYEA |
| 4988 | FQCPSGERA |
| 4989 | YSTCPNECN |
| 4990 | TVLPMIEER |
| 4991 | THNTFGYAC |
| 4992 | TNEVTCQKT |
| 4993 | SKPPGSEQS |
| 4994 | DQHCLWEAC |
| 4995 | TNMATDEYC |
| 4996 | TEEQIGPAC |
| 4997 | TGEMFNKSS |
| 4998 | TNFQADEPR |
| 4999 | VIQQFKEAC |
| 5000 | STVSYHNEA |
| 5001 | NLLPDVERG |
| 5002 | EVQPGDKCY |
| 5003 | NEDVTPCEC |
| 5004 | TWPPWCNLI |
| 5005 | TVHACKNEF |
| 5006 | NTDWLTHEA |
| 5007 | EPDANYCYS |
| 5008 | QATSTNHDA |
| 5009 | DTQNVEMAA |
| 5010 | GLVAVHEEC |
| 5011 | PEQTCYEMT |
| 5012 | NWLDGDQAC |
| 5013 | VTTVMHAIN |

In some embodiments, an RPE tissue-tropic VP capsid polypeptide comprises a 581 to 589 region having at least 75% sequence identity to any one of SEQ ID NO: 4514-SEQ ID NO: 5013. In some embodiments, an RPE tissue-tropic VP capsid polypeptide comprises a 581 to 589 region having at least 77.7% sequence identity to any one of SEQ ID NO: 4514-SEQ ID NO: 5013. In some embodiments, an RPE tissue-tropic VP capsid polypeptide comprises a 581 to 589 region having at least 85% sequence identity to any one of SEQ ID NO: 4514-SEQ ID NO: 5013. In some embodiments, an RPE tissue-tropic VP capsid polypeptide comprises a 581 to 589 region having at least 88.8% sequence identity to any one of SEQ ID NO: 4514-SEQ ID NO: 5013. In some embodiments, an RPE tissue-tropic VP capsid polypeptide comprises a 581 to 589 region having a sequence of any one of SEQ ID NO: 4514-SEQ ID NO: 5013. In some embodiments, a retina tissue-tropic VP capsid polypeptide comprises a 581 to 589 region having one or two amino acid substitutions relative to any one of SEQ ID NO: 4514-SEQ ID NO: 5013. In some embodiments, a retina tissue-tropic VP capsid polypeptide comprises a 581 to 589 region having one or two conservative amino acid substitutions relative to any one of SEQ ID NO: 4514-SEQ ID NO: 5013.

### 581 to 589 Regions for Ocular Tissue Targeting

In some embodiments, provided herein are AAV5 VP capsid polypeptide having at least one mutation in a 581 to 589 region, corresponding to residues 581 to 589 of AAV5 VP1, wherein said at least one mutation drives increased ocular tissue tropism. In some embodiments, the at least one mutation may comprise mutating one or more amino acid residues to positively charged amino acid residues (e.g., K, R, or H). In some embodiments, the at least one mutation may comprise mutating one or more negatively charged amino acid residues (e.g., E or D) to residues that are not negatively charged. In some embodiments, the 581 to 589 region comprises a sequence having at least 75% sequence identity to any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. In some embodiments, the 581 to 589 region comprises a sequence having at least 77.7% sequence identity to any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. In some embodiments, the 581 to 589 region comprises a sequence having at least 85% sequence identity to any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. In some embodiments, the 581 to 589 region comprises a sequence having at least 88.8% sequence identity to any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. In some embodiments, the 581 to 589 region comprises a sequence of any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. In some embodiments, a retina tissue-tropic VP capsid polypeptide comprises a 581 to 589 region having one or two amino acid substitutions relative to any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. In some embodiments, a retina tissue-tropic VP capsid polypeptide comprises a 581 to 589 region having one or two conservative amino acid substitutions relative to any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A viral protein (VP) capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 2014-SEQ ID NO: 2513. 2. The VP capsid polypeptide of embodiment 1, wherein the 581 to 589 region comprises a sequence of any one of SEQ ID NO: 2014-SEQ ID NO: 2513. 3. A viral protein (VP) capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 4514-SEQ ID NO: 5013. 4. The VP capsid polypeptide of embodiment 3, wherein the 581 to 589 region comprises a sequence of any one of SEQ ID NO: 4514-SEQ ID NO: 5013. 5. The VP capsid polypeptide of any one of embodiments 1-4, wherein the 581 to 589 region confers increased retinal pigment epithelium tissue-tropism compared to a wild type AAV5 VP capsid polypeptide of SEQ ID NO: 1. 6. The VP capsid polypeptide of embodiment 5, wherein the retinal pigment epithelium tissue tropism is at least 1.5-fold, at least 2-fold, at least 2.5-fold, at least 3-fold, at least 3.5-fold, at least 4-fold, at least 5-fold, at least 10-fold, at least 20-fold, at least 50-fold, at least 75-fold, at least 100-fold, at least 200-fold, or at least 500-fold higher than the wild type AAV5 VP capsid polypeptide of SEQ ID NO: 1. 7. The VP capsid polypeptide of any one of embodiments 1-6, wherein the 581 to 589 region confers increased choroid tissue-tropism compared to a wild type AAV5 VP capsid polypeptide of SEQ ID NO: 1. 8. The VP capsid polypeptide of embodiment 7, wherein the choroid tissue tropism is at least 1.5-fold, at least 2-fold, at least 2.5-fold, at least 3-fold, at least 3.5-fold, at least 4-fold, at least 5-fold, at least 10-fold, at least 20-fold, at least 50-fold, at least 75-fold, at least 100-fold, at least 200-fold, or at least 500-fold higher than the wild type AAV5 VP capsid polypeptide of SEQ ID NO: 1. 9. The VP capsid polypeptide of any one of embodiments 5-8, wherein the 581 to 589 region further confers increased retina tissue tropism compared to a wild type AAV5 VP capsid polypeptide of SEQ ID NO: 1. 10. The VP capsid polypeptide of any one of embodiments 1-9, wherein the 581 to 589 region confers decreased photoreceptor tissue-tropism compared to a wild type AAV5 VP capsid polypeptide of SEQ ID NO: 1. 11. The VP capsid polypeptide of any one of embodiments 1-10, wherein the VP capsid polypeptide comprises an amino acid sequence at least 70%, at least 80%, at least 85%, at least 90%, at least 95%, at least 97%, at least 98%, at least 98.5%, at least 99%, or at least 99.5% identical to SEQ ID NO: 1. 12. The VP capsid polypeptide of any one of embodiments 1-11, wherein the VP capsid polypeptide has a sequence of SEQ ID NO: 2, wherein residues 581 to 589 of SEQ ID NO: 2 (X¹X²X³X⁴X⁵X⁶X⁷X⁸X⁹) correspond to the 581 to 589 region. 13. The VP capsid polypeptide of any one of embodiments 1-12, wherein the VP capsid polypeptide is capable of assembling into a recombinant viral capsid. 14. The VP capsid polypeptide of any one of embodiments 1-13, comprising at least one amino acid substitution as compared to each of SEQ ID NO: 3, SEQ ID NO: 4, SEQ ID NO: 5, SEQ ID NO: 6, SEQ ID NO: 7, and SEQ ID NO: 8. 15. The VP capsid polypeptide of any one of embodiments 1-14, wherein the VP capsid polypeptide further comprises one or more mutations outside of the 581 to 589 region relative to a wild type VP capsid polypeptide of SEQ ID NO: 1. 16. The VP capsid polypeptide of any one of embodiments 1-15, wherein the 581 to 589 region confers on a recombinant viral capsid assembled from the VP capsid polypeptide improved stability compared to a wild type AAV capsid comprising a peptide of SEQ ID NO: 1. 17. The VP capsid polypeptide of any one of embodiments 1-16, wherein the 581 to 589 region confers lower toxicity upon administration to a subject compared to a wild type VP capsid polypeptide of SEQ ID NO: 1. 18. The VP capsid polypeptide of any one of embodiments 1-17, wherein the VP capsid polypeptide further comprises one or more mutations outside of the 581 to 589 region that contributes to reduced production of neutralizing antibodies relative to a wild type VP capsid polypeptide of SEQ ID NO: 1. 19. The VP capsid polypeptide of any one of embodiments 1-18, wherein the VP capsid polypeptide further comprises one or more mutations outside of the 581 to 589 region that improves manufacturability relative to a wild type VP capsid polypeptide of SEQ ID NO: 1. 20. A recombinant viral adeno-associated virus (rAAV) capsid comprising the VP capsid polypeptide of any one of embodiments 1-19, wherein the rAAV capsid preferentially targets retinal pigment epithelium tissue. 21. A recombinant viral adeno-associated virus (rAAV) capsid comprising the VP capsid polypeptide of any one of embodiments 1-19, wherein the rAAV capsid preferentially targets choroid tissue. 22. The rAAV capsid of embodiment 20 or embodiment 21, further comprising a VP2 polypeptide comprising the 581 to 589 region and a VP3 polypeptide comprising the 581 to 589 region. 23. A recombinant adeno-associated virus (rAAV), comprising the VP capsid polypeptide of any one of embodiments 1-19 assembled into a recombinant viral capsid and a payload encapsidated by the recombinant viral capsid. 24. The rAAV of embodiment 23, wherein the rAAV preferentially targets retinal pigment epithelium tissue. 25. The rAAV of embodiment 23 or embodiment 24, wherein the rAAV preferentially targets choroid tissue. 26. The rAAV of any one of embodiments 23-25, further comprising a VP2 polypeptide comprising the 581 to 589 region and a VP3 polypeptide comprising the 581 to 589 region. 27. The rAAV of any one of embodiments 23-26, wherein the payload encodes a therapeutic polynucleotide or a therapeutic peptide. 28. The rAAV of embodiment 27, wherein the payload encodes a guide RNA, a tRNA, a suppressor tRNA, a siRNA, a miRNA, an mRNA, a shRNA, a circular RNA, or an antisense oligonucleotide (ASO), a ribozyme, a DNAzyme, an aptamer, or any combination thereof. 29. The rAAV of embodiment 27, wherein the guide RNA is a CRISPR/Cas guide RNA or an ADAR guide RNA. 30. The rAAV of embodiment 27, wherein the payload encodes a component of a CRISPR/Cas system, an adenosine deaminase acting on RNA (ADAR) enzyme, a transcriptional activator, or a transcriptional repressor. 31. A pharmaceutical composition comprising the VP capsid polypeptide of any one of embodiments 1-19, the rAAV capsid of any one of embodiments 20-22, or the rAAV of any one of embodiments 23-30. 32. A method of delivering a payload to a retinal pigment epithelium tissue of a subject, the method comprising administering a recombinant adeno-associated virus (rAAV) to the subject via intravitreal administration, wherein the rAAV encapsidates the payload, and wherein the rAAV comprises a VP capsid polypeptide comprising a 581 to 589 region corresponding to residues 581 to 589 of an AAV5 VP1 polypeptide having at least 85% sequence identity to any one of SEQ ID NO: 2014-SEQ ID NO: 2513 or SEQ ID NO: 4514-SEQ ID NO: 5013. 33. A method of delivering a payload to a retinal pigment epithelium tissue of a subject, the method comprising administering a recombinant adeno-associated virus (rAAV) to the subject via intravitreal administration, wherein the rAAV encapsidates the payload, and wherein the rAAV comprises the VP capsid polypeptide of any one of embodiments 1-19. 34. A method of delivering a payload to a retinal pigment epithelium tissue of a subject, the method comprising administering a recombinant adeno-associated virus (rAAV) to the subject via intravitreal administration, wherein the rAAV encapsidates the payload, and wherein the rAAV comprises a VP capsid polypeptide comprising a 581 to 589 region corresponding to residues 581 to 589 of an AAV5 VP1 polypeptide having at least 85% sequence identity to any one of SEQ ID NO: 2014-SEQ ID NO: 2513 or SEQ ID NO: 4514-SEQ ID NO: 5013. 35. A method of delivering a payload to a choroid tissue of a subject, the method comprising administering a recombinant adeno-associated virus (rAAV) to the subject via intravitreal administration, wherein the rAAV encapsidates the payload, and wherein the rAAV comprises the VP capsid polypeptide of any one of embodiments 1-19. 36. A method of delivering a payload to a choroid tissue of a subject, the method comprising: (i) administering a recombinant adeno-associated virus (rAAV) to the subject, wherein the rAAV encapsidates the payload, and wherein the rAAV comprises a VP capsid polypeptide comprising a 581 to 589 region having at least 85% sequence identity to any one of SEQ ID NO: 2014-SEQ ID NO: 2513 or SEQ ID NO: 4514-SEQ ID NO: 5013; and (ii) delivering the payload to the choroid tissue. 37. A method of delivering a payload to a retinal pigment epithelium tissue of a subject, the method comprising: (i) administering a recombinant adeno-associated virus (rAAV) to the subject, wherein the rAAV encapsidates the payload, and wherein the rAAV comprises a VP capsid polypeptide of any one of embodiments 1-19; and (ii) delivering the payload to the retinal pigment epithelium tissue. 38. A method of delivering a payload to a choroid tissue of a subject, the method comprising: (i) administering a recombinant adeno-associated virus (rAAV) to the subject, wherein the rAAV encapsidates the payload, and wherein the rAAV comprises a VP capsid polypeptide of any one of embodiments 1-19; and (ii) delivering the payload to the choroid tissue. 39. The method of any one of embodiments 36-38, comprising administering the rAAV via intravitreal administration. 40. The method of any one of embodiments 36-38, comprising administering the rAAV via subretinal injection, topical mucosal administration, or systemic administration. 41. The method of any one of embodiments 32-40, further comprising expressing a therapeutic polypeptide or a therapeutic polynucleotide encoded by the payload. 42. The method of any one of embodiments 32-41, further comprising producing a therapeutic effect upon expression of the payload. 43. The method of embodiment 42, wherein the therapeutic effect is produced upon administration of from 1×10⁵ to 5×10¹⁴ rAAVs. 44. The method of embodiment 42 or embodiment 43, comprising administering an amount of the rAAV sufficient to produce the therapeutic effect without producing a toxicity in the subject. 45. A method of treating a condition in a subject, the method comprising administering a recombinant adeno-associated virus (rAAV) to the subject via intravitreal administration, wherein the rAAV encapsidates a payload, and wherein the rAAV comprises a VP capsid polypeptide comprising a 581 to 589 region corresponding to residues 581 to 589 of an AAV5 VP1 polypeptide having at least 85% sequence identity to any one of SEQ ID NO: 2014-SEQ ID NO: 2513 or SEQ ID NO: 4514-SEQ ID NO: 5013. 46. A method of treating a condition in a subject, the method comprising administering a recombinant adeno-associated virus (rAAV) to the subject via intravitreal administration, wherein the rAAV encapsidates a payload, and wherein the rAAV comprises the VP capsid polypeptide of any one of embodiments 1-19. 47. The method of embodiment 45 or embodiment 46, further comprising expressing the payload in a retinal pigment epithelium tissue of the subject. 48. The method of any one of embodiments 45-47, further comprising expressing the payload in a choroid tissue of the subject. 49. The method of any one of embodiments 45-48, further comprising producing a therapeutic effect in the subject. 50. A method of treating a condition in a subject, the method comprising: (i) administering a recombinant adeno-associated virus (rAAV) to the subject, wherein the rAAV encapsidates a payload, and wherein the rAAV comprises a VP capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 2014-SEQ ID NO: 2513 or SEQ ID NO: 4514-SEQ ID NO: 5013; and (ii) expressing the payload in a retinal pigment epithelium tissue of the subject, thereby treating the condition in the subject. 51. A method of treating a condition in a subject, the method comprising: (i) administering a recombinant adeno-associated virus (rAAV) to the subject, wherein the rAAV encapsidates a payload, and wherein the rAAV comprises the VP capsid polypeptide of any one of embodiments 1-19; and (ii) expressing the payload in a retinal pigment epithelium tissue of the subject, thereby treating the condition in the subject. 52. A method of treating a condition in a subject, the method comprising: (i) administering a recombinant adeno-associated virus (rAAV) to the subject, wherein the rAAV encapsidates a payload, and wherein the rAAV comprises the VP capsid polypeptide of any one of embodiments 1-19; and (ii) expressing the payload in a choroid tissue of the subject, thereby treating the condition in the subject. 53. A method of treating a condition in a subject, the method comprising: (i) administering a recombinant adeno-associated virus (rAAV) to the subject, wherein the rAAV encapsidates a payload, and wherein the rAAV comprises a VP capsid polypeptide of any one of embodiments 1-19; (ii) delivering the payload to a retinal pigment epithelium tissue of the subject; and (iii) producing a therapeutic effect in the retinal pigment epithelium tissue, thereby treating the condition. 54. A method of treating a condition in a subject, the method comprising: (i) administering a recombinant adeno-associated virus (rAAV) to the subject, wherein the rAAV encapsidates a payload, and wherein the rAAV comprises a VP capsid polypeptide of any one of embodiments 1-19; (ii) delivering the payload to a choroid tissue of the subject; and (iii) producing a therapeutic effect in the choroid tissue, thereby treating the condition. 55. The method of any one of embodiments 50-54, comprising administering the rAAV via intravitreal administration. 56. The method of any one of embodiments 50-54, comprising administering the rAAV via subretinal injection, topical mucosal administration, or systemic administration. 57. The method of any one of embodiments 45-56, wherein the condition is an ocular condition. 58. The method of embodiment 57, wherein the ocular condition is achromatopsia, macular degeneration, cataracts, choroideremia, glaucoma, optical neuropathy, Marfan syndrome, myopia, polypoidal choroidal vasculopathy, retinitis pigmentosa, Stargardt disease, Usher syndrome, Leber congenital amaurosis, Leber hereditary optical neuropathy, Uveal melanoma, or X-linked retinoschisis. 59. The method of embodiment 57 or embodiment 58, wherein the ocular condition is Stargardt disease. 60. The method of any one of embodiments 32-59, further comprising delivering the rAAV to a retinal pigment epithelium tissue with higher tissue tropism than a wild type AAV5 capsid comprising a peptide of SEQ ID NO: 1. 61. The method of any one of embodiments 39-53, further comprising delivering the rAAV to a retinal pigment epithelium tissue with higher tissue tropism than for photoreceptor tissue. 62. The method of any one of embodiments 39-54, comprising delivering the rAAV to a retinal pigment epithelium tissue with at least 2.5-fold, at least 3-fold, at least 3.5-fold, at least 4-fold, at least 5-fold, at least 10-fold, at least 20-fold, at least 50-fold, at least 75-fold, at least 100-fold, at least 200-fold, at least 500-fold higher, or at least 1000-fold higher tissue tropism than a wild type AAV5 capsid comprising a peptide of SEQ ID NO: 1. 63. The method of any one of embodiments 32-59, further comprising delivering the rAAV to a choroid tissue with higher tissue tropism than a wild type AAV5 capsid comprising a peptide of SEQ ID NO: 1. 64. The method of any one of embodiments 39-53, further comprising delivering the rAAV to a choroid tissue with higher tissue tropism than for photoreceptor tissue. 65. The method of any one of embodiments 39-54, comprising delivering the rAAV to a choroid tissue with at least 2.5-fold, at least 3-fold, at least 3.5-fold, at least 4-fold, at least 5-fold, at least 10-fold, at least 20-fold, at least 50-fold, at least 75-fold, at least 100-fold, at least 200-fold, at least 500-fold higher, or at least 1000-fold higher tissue tropism than a wild type AAV5 capsid comprising a peptide of SEQ ID NO: 1. 66. The method of any one of embodiments 39-65, comprising administering from 1×10⁵ to 5×10¹⁴ rAAVs. 67. The method of any one of embodiments 39-66, wherein the payload encodes a therapeutic protein, therapeutic polynucleotide, a guide RNA, a tRNA, a suppressor tRNA, a siRNA, a miRNA, an mRNA, a shRNA, a circular RNA, an antisense oligonucleotide (ASO), a ribozyme, a DNAzyme, an aptamer, or any combination thereof. 68. The method of embodiment 67, wherein the guide RNA is a CRISPR/Cas guide RNA or an ADAR guide RNA. 69. The method of embodiment 67, wherein the therapeutic protein is selected from the group consisting of CNGB3, NOS2A, CFH, CF, C2, C3, CFB, HTRA1/LOC, MMP-9, TIMP-3, SLC16A8, GEMIN4, CYP51A1, RIC1, TAPT1, TAF1A, WDR87, APE1, MIP, Cx50, GJA3, GJA8, CRYAA, CRYBB2, PRX, POLR3B, XRCC1, ZNF350, EPHA2, REP1, CALM2, MPP-7, Optineurin, LOX1, CYP1B1, CAV1/2, MYOC, PITX2, FOXC1, PAX6, LTBP2, Complex I, ND, OPA1, RPE65, FBN1, TGFBR2, MTHFR, MTR, MTRR, HGF, C-MET, UMODL1, MMP-1/2, CBS, IGF-1, UHRF1BP1L, PTPRR, PPFIA2, P4HA2, SERPING1, PEDF, ARMS2-HTRA1, FGD6, ABCG1, LOC387715, CETP, NRL, RDH12, PRPH2 (RDS), RHO, RPGR, SNRNP200, NR2E3, IMPDH1, CRX, HK1, IMPDH2, PRPF3, AGBL5, ABCA1, ABCA4, CRB1, USH2A, NRP1, ND4, RLBP1, PTEN, BAP1, GNAQ, GNA11, DDEF1, SF3B1, EIF1AX, CDKN2A, p14ARF, HERC2/OCA2, VEGF, and RS1. 70. The method of embodiment 67, wherein the therapeutic polynucleotide targets an mRNA encoding a protein selected from the group consisting of CNGB3, NOS2A, CFH, CF, C2, C3, CFB, HTRA1/LOC, MMP-9, TIMP-3, SLC16A8, GEMIN4, CYP51A1, RIC1, TAPT1, TAF1A, WDR87, APE1, MIP, Cx50, GJA3, GJA8, CRYAA, CRYBB2, PRX, POLR3B, XRCC1, ZNF350, EPHA2, REP1, CALM2, MPP-7, Optineurin, LOX1, CYP1B1, CAV1/2, MYOC, PITX2, FOXC1, PAX6, LTBP2, Complex I, ND, OPA1, RPE65, FBN1, TGFBR2, MTHFR, MTR, MTRR, HGF, C-MET, UMODL1, MMP-1/2, CBS, IGF-1, UHRF1BP1L, PTPRR, PPFIA2, P4HA2, SERPING1, PEDF, ARMS2-HTRA1, FGD6, ABCG1, LOC387715, CETP, NRL, RDH12, PRPH2 (RDS), RHO, RPGR, SNRNP200, NR2E3, IMPDH1, CRX, HK1, IMPDH2, PRPF3, AGBL5, ABCA1, ABCA4, CRB1, USH2A, NRP1, ND4, RLBP1, PTEN, BAP1, GNAQ, GNA11, DDEF1, SF3B1, EIF1AX, CDKN2A, p14ARF, HERC2/OCA2, VEGF, or RS1. 71. The method of embodiment 67, wherein the payload encodes a therapeutic polynucleotide targeting ABCA1, ABCA4, or CRB1 or a transgene encoding ABCA1, ABCA4, or CRB1. 72. The method of embodiment 67, wherein the payload encodes a component of a CRISPR/Cas system, an adenosine deaminase acting on RNA (ADAR) enzyme, a transcriptional activator, or a transcriptional repressor. 73. The method of embodiment 72, wherein the component of the CRISPR/Cas system comprises a Cas3, a Cas8, a Cas10, a Cas9, a Cas4, a Cas12, a Cas13, a guide RNA, or a combination thereof. 74. The method of embodiment 72, wherein the ADAR enzyme is ADAR1 or ADAR2. 75. The method of embodiment 72, wherein the transcriptional activator is VP64. 76. The method of embodiment 72, wherein the transcriptional repressor is KRAB. 77. The method of any one of embodiments 67-76, further comprising expressing a therapeutic polypeptide or a therapeutic polynucleotide encoded by the payload. 78. The method of any one of embodiments 67-77, comprising expressing the therapeutic polypeptide or the therapeutic polynucleotide at a higher level in a retinal pigment epithelium tissue compared to a wild type AAV5 capsid comprising a peptide of SEQ ID NO: 1. 79. The method of embodiment 78, comprising expressing the therapeutic polypeptide or the therapeutic polynucleotide in a retinal pigment epithelium tissue at a level that is at least 3-fold, at least 3.5-fold, at least 4-fold, at least 5-fold, at least 10-fold, at least 20-fold, at least 50-fold, at least 75-fold, at least 100-fold, at least 200-fold, at least 500-fold higher, or at least 1000-fold higher compared to the wild type AAV5 capsid. 80. The method of any one of embodiments 32-79, comprising producing a toxicity in the subject that is lower than a toxicity produced upon administration of a comparable number of wild type AAV5 capsids comprising a VP capsid polypeptide of SEQ ID NO: 1. 81. The method of any one of embodiments 32-80, comprising producing a toxicity in the subject that is lower than a toxicity produced upon administration of a number of wild type AAV5 capsids comprising a VP capsid polypeptide of SEQ ID NO: 1 sufficient to deliver a comparable number of payloads to a retinal pigment epithelium tissue. 82. The method of any one of embodiments 32-81, comprising producing a concentration of neutralizing antibodies in the subject that is lower than a concentration of neutralizing antibodies produced upon administration of a comparable number of wild type AAV5 capsids comprising a VP capsid polypeptide of SEQ ID NO: 1. 83. The method of any one of embodiments 32-82, comprising producing a concentration of neutralizing antibodies in the subject that is lower than a concentration of neutralizing antibodies produced upon administration of a comparable number of wild type AAV2 capsids. 84. The method of any one of embodiments 32-83, comprising producing a concentration of neutralizing antibodies in the subject that is lower than a concentration of neutralizing antibodies produced upon administration of a comparable number of wild type AAV8 capsids. 85. The method of any one of embodiments 32-84, comprising producing a concentration of neutralizing antibodies in the subject that is lower than a concentration of neutralizing antibodies produced upon administration of a comparable number of wild type AAV9 capsids. 86. The method of any one of embodiments 32-85, comprising producing a concentration of neutralizing antibodies in the subject that is lower than a concentration of neutralizing antibodies produced upon administration of a number of wild type AAV5 capsids comprising a VP capsid polypeptide of SEQ ID NO: 1 sufficient to deliver a comparable number of payloads to a retinal pigment epithelium tissue.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### Example 1

Identification of 581 to 589 Region Sequences that Confer Ocular Tissue Tropism

This example describes identification of 581 to 589 region sequences that confer ocular tissue tropism. The high throughput capsid engineering system is schematized in FIG. 1.

Tissues were harvested and transducing capsid genes are labeled with unique molecular identifiers and barcodes. These variant sequences/unique molecular identifiers (UMIs)/barcodes were parameterized, and machine learning algorithms were used to identify deterministic features of specific tissue targeting/detargeting capsids.

FIG. 2A provides a side view (top panel) and top view (bottom panel) of key residues of known AAV capsids that have been shown to interact with target cells. FIG. 2B illustrates salient features of the AAV capsid library, showing the region of introduced diversity, with residue numbering corresponding to the numbering of amino acids in AAV5 VP1. The library plasmid includes the invariant rep and diversified cap coding sequences between AAV inverted terminal repeats (ITRs; a cis-packaging approach).

The recombinant AAV library was administered to two non-human primates (NHPs), one male and one female. The library was administered via bilateral intravitreal injection of 3.2×10¹² viral genomes in 50 μL per eye as follows. Animals were sedated/anesthetized to effect and placed in dorsal recumbency. Topical Proparacaine was applied to the eye. The conjunctival fornices were flushed with a 1:50 dilution of betadine solution/saline and the eyelid margins swabbed with undiluted 5% betadine solution. The eye was draped, and a wire eyelid speculum placed. A caliper was used to mark a spot 3.0 mm posterior to the limbus on the inferotemporal bulbar conjunctiva. The conjunctiva at the marked spot was swabbed with undiluted 5% betadine solution. Conjunctival forceps were used to fixate the globe position while a STACLEAR™ syringe with 31 g needle was inserted at the marked spot, through the sclera and advanced into the vitreous humor. The injection needle was positioned to face the posterior axis of the globe and 50 μL of AAV5 library was delivered into the vitreous by slowly depressing the syringe plunger. The needle was held in place for at least 30 seconds to lessen reflux of the injected material. Subsequently, the needle was removed and the episcleral tissues approximated to the site of insertion grasped with the conjunctival forceps to further lessen reflux of the injected material. An identical injection procedure was then performed on the contralateral eye.

Four weeks post-injection, the animals were euthanized, and tissues were collected for analysis. For the ocular dissections, both eyes first had a maximum volume of aqueous and vitreous humor collected. The retina was separated from the retinal pigment epithelium (RPE)/choroid. The RPE/choroid was frozen in liquid nitrogen as a stand-alone tissue. The retina was further dissected into 5 pieces; superior, inferior, temporal, nasal, central (including fovea and optic disc) and frozen in liquid nitrogen.

The frozen specimens were mechanically dissociated in TRIZOL™ Reagent using QIAGEN® GENTLEMACS™ as follows. Tissue was homogenized using a GENTLEMACS™ dissociator. The tissue was spun down at 3000×g for 2-3 minutes. A 15 ml MAXTRACT® QIAGEN® protocol was used to extract the AAV episomal DNA. Samples were added to MAXTRACT® High Density Tubes. Between 1 and 6 mL were added to 15 mL tubes and between 5 and 20 nM were added to 50 mL tubes, avoiding any insoluble material. In some instances, GLYCOBLUE™ was added as a co-precipitant. Lysis reagent and 0.2 mL chloroform per mL lysis reagent was added to each tube, and the tubes were capped and thoroughly mixed for at least 15 seconds to mix the organic and aqueous phases to form a pseudo-homogeneous suspension. Tubes were incubated at room temperature for 2 to 3 min. Following incubation, the samples were centrifuged at 1500×g for 5 min at 4° C. to separate the phases or until sample is completely phased (pink is no longer visible in the aqueous layer). The resulting sample contained three phases. The upper of the three phases was a colorless aqueous phase containing cellular RNA and AAV episomal DNA. The upper layer was transferred to a new tube by gently decanting into the new tube. For precipitation of RNA/episomal DNA from the aqueous phase, 0.5 mL of isopropanol was added per 1 mL of lysis reagent used previously and mixed thoroughly. The tubes were incubated at room temperature for 10 min and centrifuged at 4200×g for 10 to 15 min at 4° C. A swinging bucket rotor was used to pellet the RNA and episomal DNA to the bottom of the tube. Following centrifugation, the supernatant was carefully discarded without disrupting the pellet, which was visible as a gel-like white (or blue if co-precipitant was used) pellet at the bottom of the tube. At least 1 mL of freshly made 75% ethanol per 1 mL lysis reagent used previously was added. Samples were mixed by vortexing for a few seconds then centrifuged at 4200×g for 5 min at 4° C. The supernatant was discarded, and the remaining liquid was aspirated following a quick spin. The pellet, containing RNA, was briefly air-dried for about 5 minutes without completely drying the pellet. The RNA pellet was redissolved in 250 μL of RNase-free water prewarmed to 37° C. In some instances, the RNA was treated with 6 μL per 100 μL RNase Cocktail Enzyme Mix (THERMO FISHER SCIENTIFIC®; AM2286) at 37° C. for 30 min. to remove RNA. The treated mixture was purified with a Zymo DNA Clean and Concentrator kit-25 (D4033) following the manufacturer's protocol.

Next generation sequencing (NGS) of recovered AAV episomal DNA was performed using a semi-nested 3-round PCR amplification that appends unique molecular identifiers (UMIs) in the first round of PCR. The completed amplicon contained both UMIs and NGS adapters with dual indexes for sample demultiplexing. The first round of PCR was performed as follows. Four reactions were prepared per tissue sample. Each 100 μL PCR reaction contained 20 μL episomal DNA per reaction as a template. PCR cycling was performed (1. 98° C. for 30s; 2. 98° C. for 15s; 3. 66.6° C. annealing for 45s; 4. 72° C. for 45s; 5. Repeat steps 2-4 for 5 cycles; 6. 72° C. for 2 min). The amplified product was cleaned using CYTIVA® SERA-MAG™ Select bead clean-up following the manufacturer's protocol (1:1 bead:sample).

The second round of PCR was performed as follows. 23 μl of cleaned up PCR 1 reaction was used as a template. 2 μL of primers (10 μM) and 25 μL of 2× master mix Phusion polymerase with HF buffer (NEB, M0531L) was added per reaction. PCR cycling was performed (1. 98° C. for 30s; 2. 98° C. for 10s; 3. 62° C. for 30s; 4. 72° C. for 20s; 5. Repeat steps 2-4 for 18 cycles; 6. 72° C. for 5 min; 7. 4° C. hold). The amplified product was cleaned using CYTIVA® SERA-MAG™ Select bead clean-up following the manufacturer's protocol (0.75:1 bead:sample).

Quantitative PCR (qPCR) with SYBR was performed as follows. 4 μL of cleaned up PCR 2 reaction was used as a template. 1 μL of primers at 20 μL of master mix (containing SYBR) was added. PCR cycling was performed (1. 98° C. for 30s; 2. 98° C. for 10s; 3. 72° C. for 20s and plate read; 4. Repeat steps 2 and 3 for 29 more times; 5. 72° C. for 5m). Amplification and cycle threshold (Ct) values were recorded. Following determination of Ct values, qPCR was repeated as described above but with endpoint at 3-4 cycles above Ct value for each sample. The amplified product was cleaned using CYTIVA® SERA-MAG™ Select bead clean-up following the manufacturer's protocol (1.25:1 bead:sample).

The libraries were pooled and checked for quality. Presence of a 296 bp band for each sample was verified by gel electrophoresis. Samples were quantified on a QUBIT™ flex with High Sensitivity reagents, pooling equal amounts (in ng) together. A TAPESTATION™ automated electrophoresis system was used to confirm quality and quantify each pool. Samples were sequenced on an ISEQ™ for pooling and quality control of indexes. In some instances, depending on library diversity and depth of sequence desired, sequencing was performed on an ILLUMINA® HISEQ™ or NOVASEQ™.

To isolate the capsid variant sequence to use for all analyses, raw demultiplexed data were processed using the following procedure executed using NEXTFLOW™ and custom PYTHON® scripts. Paired end sequencing reads were merged using Fastp v0.21.0 and the following parameters: --correction -merge -average_qual 30. This removed reads with an average sequencing quality score <Q30 and corrected base calls using the read with the higher Phred quality score at that position. Capsid variant sequences were extracted by aligning with zero mismatches in the five nucleotide sequences flanking the variant sequence, and UMI sequences were isolated as the first 12 nucleotides of the read. Extracted variant sequences were then conservatively clustered within an edit distance of four to collapse/suppress error. The sequence at the center of each cluster (i.e., the consensus sequence) was then translated into its amino acid sequence, and data were filtered respect to sequence quality and abundance. Sequences were removed if containing one of the codons excluded from library synthesis (‘CTC’, ‘CTA’, ‘TTA’, ‘AGG’, ‘CGA’, ‘CGT’, ‘TAA’, ‘TAG’, ‘TGA’).

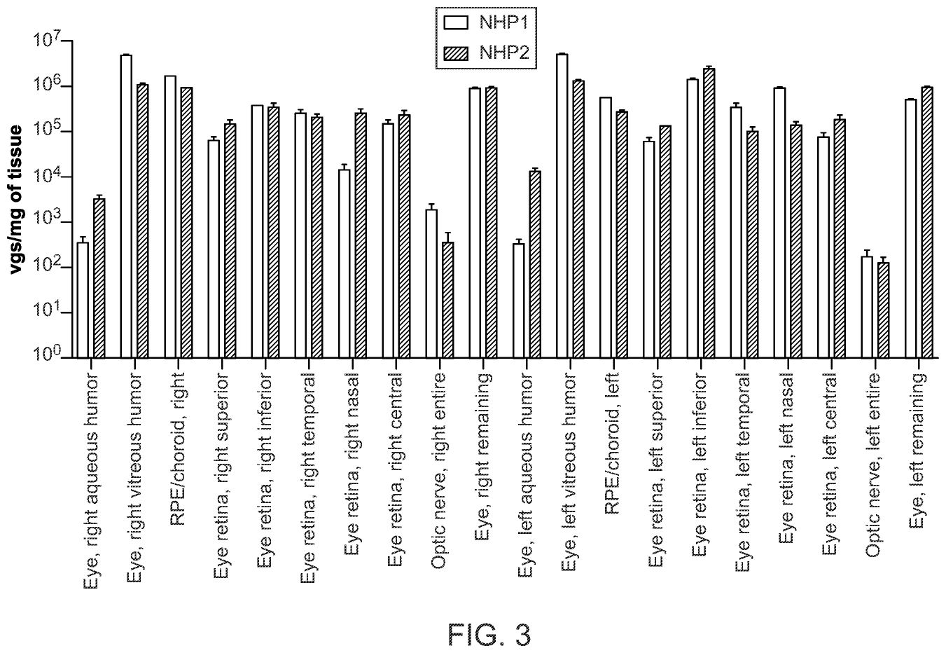

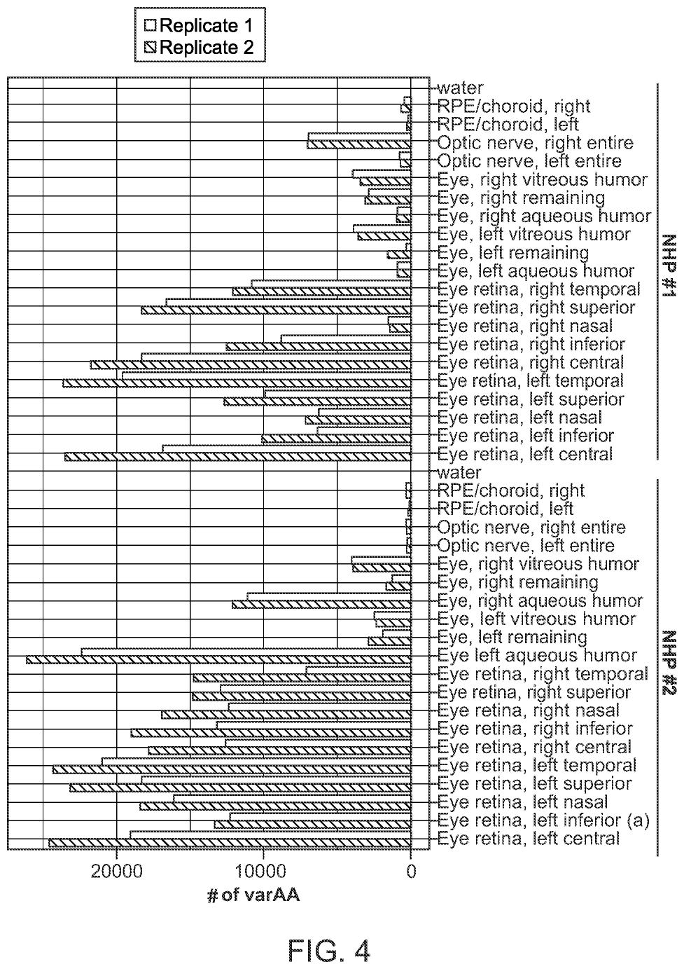

Analysis was performed using the R programming language, and the number of unique variant sequences within each tissue was determined, as shown in FIG. 4. Number of viral genomes recovered per mg from each tissue is shown in FIG. 3.

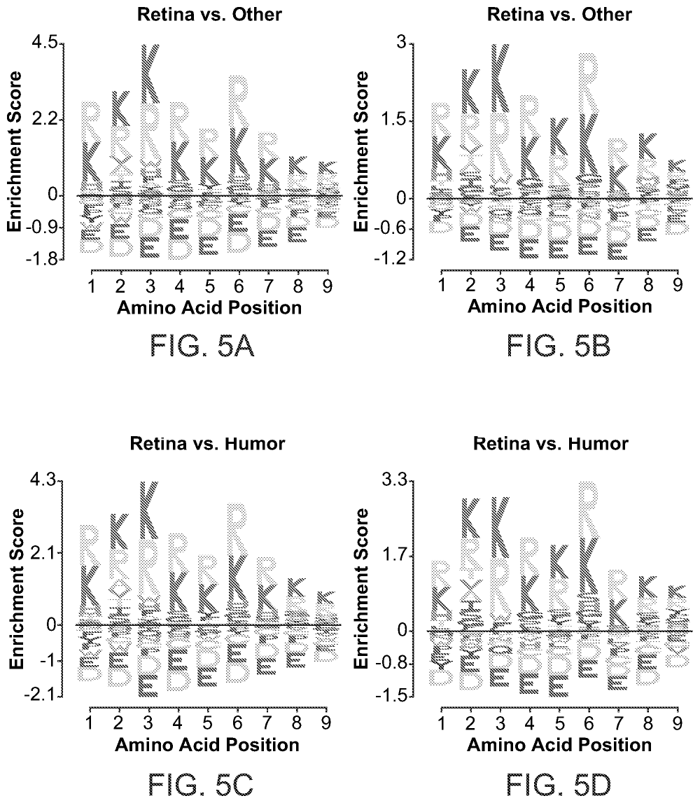

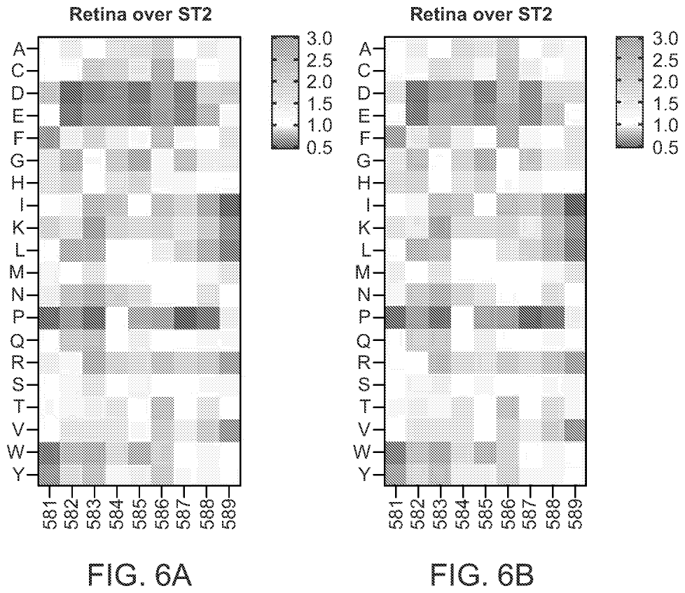

Enrichment of amino acid residues at each position in the 581 to 589 region (AAV5 VP1 581-589) was determined for the retina in comparison to various background amino acid frequencies, including all other eye tissues (FIG. 5A and FIG. 5B) or the aqueous/vitreous humor (FIG. 5C and FIG. 5D). FIG. 6A and FIG. 6B show heatmaps of amino acid enrichment and depletion at each residue position of the 581 to 589 region (581 to 589, corresponding to positions in the AAV5 VP1 sequence) for the two NHPs, as fold increased/decreased over the input viral library frequency (“ST2”). As seen in FIG. 5A-FIG. 5D, FIG. 6A, and FIG. 6B, positively charged amino acid residues (e.g., K and R) are favored for retinal tissue tropism.

### Example 2

AAV5 Variants with Tissue Tropism in Eye Tissues

This example describes engineered AAV5 variants with ocular tissue tropism that are discovered using the methods and systems described in EXAMPLE 1.

Positively charged residues, including lysine (K), arginine (R), and histidine (H), were identified as promoting ocular tissue tropism when included in the VP capsid polypeptide 581 to 589 region. Accordingly, an ocular tissue-tropic VP capsid polypeptide may comprise a K, R, or H amino acid residue at one or more of X¹, X², X³, X⁴, X⁵, X⁶, X⁷, X⁸, or X⁹ of SEQ ID NO: 2. Conversely, negatively charged residues, including aspartic acid (D) and glutamic acid (E), were identified as disfavoring ocular tissue tropism when included in the VP capsid polypeptide 581 to 589 region. Accordingly, an ocular tissue-tropic VP capsid polypeptide may comprise fewer D or E amino acid residues in the 581 to 589 region than a wild type VP capsid polypeptide (e.g., SEQ ID NO: 1), or D and E residues may be excluded from the 581 to 589 region entirely.

The present disclosure, thus, provides for rAAVs composed of engineered AAV5 VP1 capsid polypeptides having a SEQ ID NO: 2, wherein the X¹, X², X³, X⁴, X⁵, X⁶, X⁷, X⁸, and X⁹ are independently selected from A, R, N, D, C, E, Q, G, H, I, L, K, M, F, P, S, T, W, Y, and V. Also encompassed herein are rAAVs composed of engineered AAV5 VP2 capsid polypeptides and engineered AAV5 VP3 capsid polypeptides having the 581 to 589 regions described herein at the regions in AAV5 VP2 (amino acid residues 445 to 453) and AAV5 VP3 (amino acid residues 389 to 397) corresponding to the amino acids in the AAV5 VP1 581 to 589 region.

From the primary screen described in EXAMPLE 1, certain generalizable trends were also observed for AAV5 variants with ocular tissue tropism. The presence of basic amino acid residues, including K, R, and H, in the 581 to 589 region was favorable for retina tissue tropism and enhanced capsid preference for retina tissue. Increasing the number of basic amino acid residues in the 581 to 589 region increased capsid preference for retina tissue. Basic residues had the largest favorable impact on retina tissue tropism when positioned at position X³ of the 581 to 589 region, with reference to SEQ ID NO: 2, followed by positions X⁶, X¹, and X⁴, in order of impact. Conversely, the presence of acidic amino acid residues, including D and E, in the 581 to 589 region was unfavorable for retina tissue tropism and decreased capsid preference for retina tissue. Furthermore, capsids with 581 to 589 regions containing two or more basic amino acid residues exhibited greater preference for retina tissue with fewer acidic amino acid residues. Increasing the number of acidic amino acid residues in a 581 to 589 region decreased preference for retina tissue in capsids with 581 to 589 regions containing two or more basic amino acid residues.

### Example 3

### Selection of Prospective Tissue-Tropic Capsid Variants for Secondary Screen

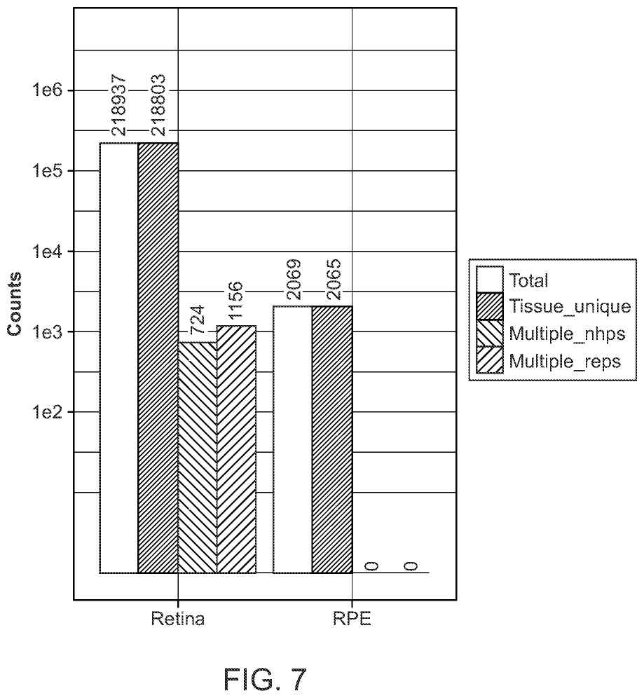

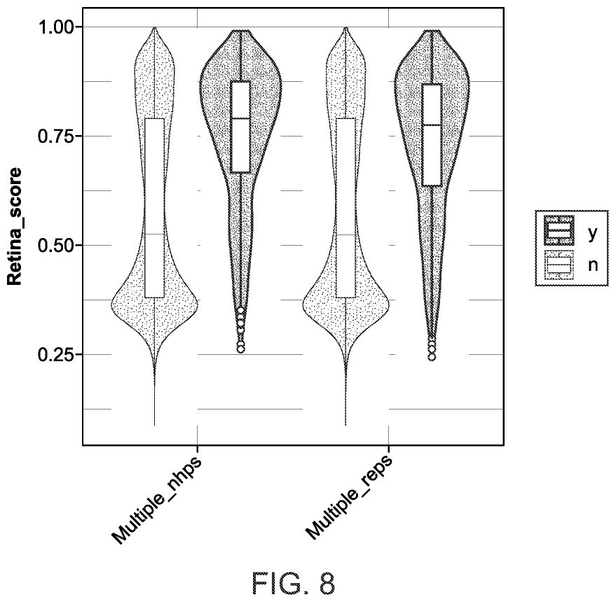

This example describes the selection of prospective tissue-tropic capsid variants for secondary screening. Prospective retinal pigment epithelium (RPE) tissue-tropic and retinal tissue-tropic AAV5 capsid polypeptide variants were identified using the primary screen described in EXAMPLE 1. An AAV5 capsid polypeptide variant was identified as a prospective tissue-tropic variant if it was found only in that tissue of a non-human primate (NHP) following intravitreal administration. Of the 218,937 AAV5 capsid polypeptide variants identified in retinal tissue, 218,803 AAV5 capsid polypeptide variants were found exclusively in retinal tissue, as shown in FIG. 7. From those variants, 1156 were found exclusively in retinal tissue across multiple replicates, of which 724 were found exclusively in retinal tissue in multiple NHPs. Prospective retinal tissue-tropic variants were prioritized for inclusion in the secondary screen if they were observed multiple times in retinal tissue. As shown in FIG. 8, variants with multiple observations (“y”), either in multiple NHPs or multiple replicates within a particular NHP, had higher predictive probabilities (“retina_score”) of exhibiting retina-specific tropism compared to variants having a single observation (“n”), thus providing additional support for their inclusion in the secondary screen. Sequences found exclusively in retinal tissue across multiple replicates, corresponding to AAV5 capsid polypeptide variants with 581 to 589 regions having sequences of SEQ ID NO: 738-SEQ ID NO: 1169, were selected as prospective retinal tissue-tropic AAV5 capsid polypeptide variants for inclusion in the secondary screen. Sequences found exclusively in retinal tissue across multiple NHPs, corresponding to AAV5 capsid polypeptide variants with 581 to 589 regions having sequences of SEQ ID NO: 14-SEQ ID NO: 737, were selected as prospective retinal tissue-tropic AAV5 capsid polypeptide variants for inclusion in the secondary screen. Additionally, observed tissue-unique prospective retina tissue-tropic AAV5 capsid polypeptide variants with 581 to 589 regions having sequences of SEQ ID NO: 1170-SEQ ID NO: 2013 were selected using machine learning for inclusion in the secondary screen. A total of 724 tissue-unique prospective retina tissue-tropic variants observed in multiple NHP were included in the secondary screen. A total of 432 tissue-unique prospective retina tissue-tropic variants observed in multiple replicates were included in the secondary screen. A total of 844 other observed prospective retina tissue-tropic variants, top-ranked by machine learning, but only observed in one replicate of one NHP, were included in the secondary screen.

As shown in FIG. 7, 2069 AAV5 capsid polypeptide variants were identified in RPE tissue and 2065 AAV5 capsid polypeptide variants were identified exclusively in RPE tissue. Observed RPE tissue-tropic AAV5 capsid polypeptide variants with 581 to 589 regions having sequences of SEQ ID NO: 2014-SEQ ID NO: 2513 were selected using machine learning for inclusion in the secondary screen. A total of 500 top ML-ranked tissue-unique prospective RPE tissue-tropic variants, having high predictive probability of being RPE-tropic and low predictive probability of being retina-tropic, were included in the secondary screen.

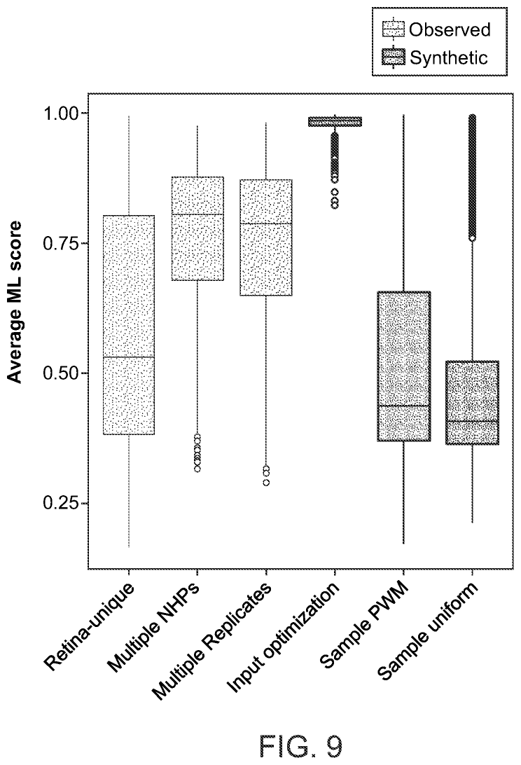

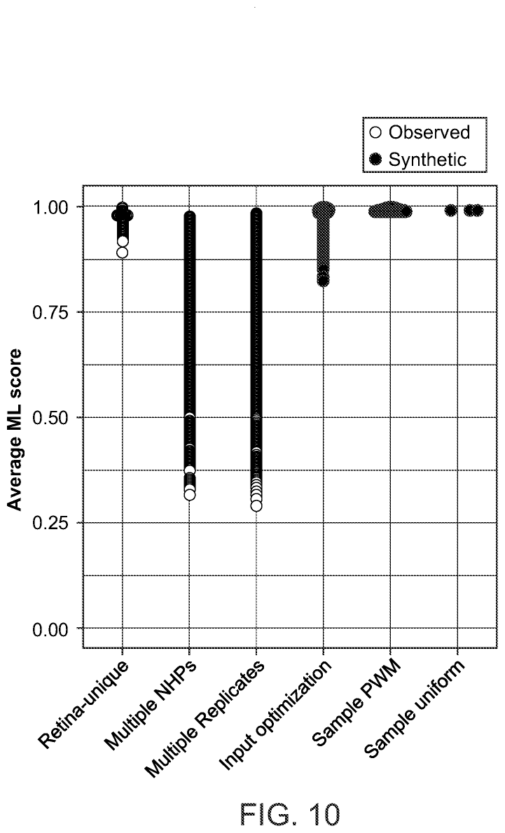

Additional prospective RPE tissue-tropic and retinal tissue-tropic engineered AAV5 capsid polypeptides to be included in the secondary screen were selected based on a machine learning analysis of the results of the primary screen performed in EXAMPLE 1. Synthetic AAV5 capsid polypeptide variants predicted to have retina tissue tropism or RPE tissue tropism were produced using input optimization through generative convolutional neural network (CNN) modeling; by sampling position weight matrices (PWMs) of amino acid frequencies for a tissue; or by sampling a uniform distribution of amino acid frequencies. These variants were then scored using the machine learning models trained using the observed capsid variants, with the top ML-ranked variants being selected for inclusion in the secondary screen. For retina tissue, synthetic sequences identified using input optimization had the highest predictive probabilities (“Average ML Score”) of having retina-specific tropism, as shown in FIG. 9. FIG. 9 shows the distributions of predicted probabilities of all variants with multiple observations in multiple NHPs or multiple replicates compared to all synthetic retina-tropic variants generated by machine learning, before secondary screen selection. FIG. 10 shows the distributions of predicted probabilities of the variants selected for inclusion in the secondary screen, categorized by how the variant was identified.

AAV5 capsid polypeptide variants with 581 to 589 regions having sequences of SEQ ID NO: 2514-SEQ ID NO: 3575 were generated using convolutional neural network (CNN) input optimization as prospective retina tissue-tropic and selected for inclusion in the secondary screen. AAV5 capsid polypeptide variants with 581 to 589 regions having sequences of SEQ ID NO: 3576-SEQ ID NO: 4510 were generated by sampling PWMs, followed by scoring these sequences and selecting high-ranking sequences as prospective retina tissue-tropic for inclusion in the secondary screen. AAV5 capsid polypeptide variants with 581 to 589 regions having sequences of SEQ ID NO: 4511-SEQ ID NO: 4513 were generated by sampling a uniform distribution, followed by scoring these sequences as retina tissue-tropic for inclusion in the secondary screen. When selecting sequences for further screening, variants generated from sampling PWMs and variants generated from sampling a uniform distribution were considered together. A total of 2000 synthetic prospective retina tissue-tropic variants were included in the secondary screen, including 1062 input optimized sequences, 935 sequences from sampling PWMs, and 3 sequences identified from uniform distribution sampling.

AAV5 capsid polypeptide variants with 581 to 589 regions having sequences of SEQ ID NO: 4514-SEQ ID NO: 5013 were generated by sampling PWMs, followed by scoring these sequences and selecting high-ranking sequences as prospective RPE tissue-tropic for inclusion in the secondary screen. A total of 500 synthetic prospective RPE tissue-tropic variants were included in the secondary screen.

### Example 4

### Secondary Screen to Identify Ocular Tissue-Tropic Engineered AAV Capsids

This example describes a secondary screen to identify ocular tissue-tropic engineered AAV capsids. Prospective tissue-tropic capsids identified from an in vivo primary screen, through machine learning, or both, such as the retina tissue-tropic and RPE tissue-tropic capsids identified in EXAMPLE 3, are introduced into an in vivo secondary screen to confirm tissue-tropic behavior of the identified capsids. The secondary screen includes capsids observed in the primary screen in RPE, choroid, retina, or combinations thereof. The barcoded secondary screen capsid library, including wild type AAV controls, is injected into a non-human primate (NHP) by intravitreal injection. Following injection, ocular tissues, including RPE/choroid, retina, vitreous humor, aqueous humor, and optic nerve, are isolated from the NHP, and the barcoded sequences are quantified. RPE and choroid tissues are isolated and analyzed together.

To identify RPE-targeting capsids in the functional screen, two versions of every candidate capsid in the library are encoded. The first version includes a payload sequence under transcriptional control of a universal promoter (e.g., CAG). The second version includes a payload sequence under transcriptional control of an RPE-specific promoter (e.g., BEST1). Capsids that infect RPE will show RNA expression from both the RPE-specific and universal promoters, whereas capsids infecting other cells will show expression from the CAG promoter and lack expression from the BEST1 promoter. Capsids found in RPE/choroid tissue that express from both the RPE-specific and universal promoters are identified as RPE tissue-tropic capsids. Capsids found in retina tissue are identified as retina tissue-tropic capsids.

### Example 5

Treatment of an Eye Disease or Condition with an AAV5-Derived Virion Encapsidating a Therapeutic Payload

This example describes treatment of an ocular disease or condition with a variant AAV5-derived virion having any one of the engineered ocular tissue-tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581 to 589 region, corresponding to residues 581 to 589 in the VP1 capsid polypeptide, and where the polynucleotide sequence encodes for a variant AAV5 Cap comprising a 581 to 589 region that confers retinal tissue tropism, photoreceptor cell targeting, retinal pigment epithelial cell targeting, or combinations thereof. The 581 to 589 region has a sequence of any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. Each such variant is detargeted for vitreous humor and aqueous humor. The therapeutic payload is a guide RNA or miRNA targeting an mRNA encoded for by a gene implicated in the ocular disease or condition, or the therapeutic payload is a transgene. The variant AAV5 virion encapsidating the payload is administered to a subject. The subject is a human or non-human animal. The route of administration is an intravitreal route of administration. The intravitreal route of administration is intravitreal injection. Upon administration to the subject, the variant AAV5 virions encapsidating the therapeutic payload exhibit enhanced ocular tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of the ocular disease or condition is alleviated, or the subject is cured.

### Example 6

Treatment of Macular Degeneration with an AAV5-Derived Virion Encapsidating a Therapeutic Payload

This example describes treatment of wet age-related macular degeneration with a variant AAV5-derived virion having any one of the engineered ocular tissue-tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581 to 589 region, corresponding to residues 581 to 589 in the VP1 capsid polypeptide, and where the polynucleotide sequence encodes for a variant AAV5 Cap comprising a 581 to 589 region conferring retinal tissue targeting, retinal pigment epithelial cell targeting, or both. The 581 to 589 region has a sequence of any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. Each such variant is detargeted for vitreous humor and aqueous humor. The therapeutic payload is a guide RNA targeting an mRNA encoded for by a gene implicated in wet age-related macular degeneration, or the therapeutic payload is a transgene. The mRNA targeted by the guide RNA is an mRNA encoded for by a NOS2A, CFH, CF, C2, C3, CFB, HTRA1/LOC, MMP-9, TIMP-3, or SLC16A8 gene. The transgene is a gene encoding for an anti-VEGF antibody. The variant AAV5 virion encapsidating the payload is administered to a subject. The subject is a human or non-human animal. The route of administration is an intravitreal route of administration. The intravitreal route of administration is intravitreal injection. Upon administration to the subject, the variant AAV5 virions encapsidating the therapeutic payload exhibit enhanced ocular tissue tropism compared with wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of wet age-related macular degeneration is alleviated, or the subject is cured.

### Example 7

Treatment of Glaucoma with an AAV5-Derived Virion Encapsidating a Therapeutic Payload

This example describes treatment of glaucoma with a variant AAV5-derived virion having any one of the engineered ocular tissue-tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581 to 589 region, corresponding to residues 581 to 589 in the VP1 capsid polypeptide, and where the polynucleotide sequence encodes for a variant AAV5 Cap comprising a 581 to 589 region that confers retinal tissue targeting, retinal pigment epithelial cell targeting, or both. The 581 to 589 region has a sequence of any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. Each such variant is detargeted for vitreous humor and aqueous humor. The therapeutic payload is a guide RNA targeting an mRNA encoded for by a gene implicated in glaucoma, or the therapeutic payload is a transgene. The mRNA targeted by the guide RNA is an mRNA encoded for by a CALM2, MPP-7, Optineurin, LOX1, CYP1B1, CAV1/2, MYOC, PITX2, FOXC1, PAX6, CYP1B1, or LTBP2 gene. The transgene is optineurin. The variant AAV5 virion encapsidating the payload is administered to a subject. The subject is a human or non-human animal. The route of administration is an intravitreal route of administration. The intravitreal route of administration is intravitreal injection. Upon administration to the subject, the variant AAV5 virions encapsidating the therapeutic payload exhibit enhanced ocular tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of glaucoma is alleviated, or the subject is cured.

### Example 8

Treatment of Retinitis Pigmentosa with an AAV5-Derived Virion Encapsidating a Therapeutic Payload

This example describes treatment of retinitis pigmentosa with a variant AAV5-derived virion having any one of the engineered ocular tissue-tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581 to 589 region, corresponding to residues 581 to 589 in the VP1 capsid polypeptide, and where the polynucleotide sequence encodes for a variant AAV5 Cap comprising a 581 to 589 region that confers retinal tissue targeting, retinal pigment epithelial cell targeting, or both. The 581 to 589 region has a sequence of any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. Each such variant is detargeted for vitreous humor and aqueous humor. The therapeutic payload is a guide RNA targeting an mRNA encoded for by a gene implicated in the retinitis pigmentosa, or the therapeutic payload is a transgene. The mRNA targeted by the guide RNA is an mRNA encoded for by a NRL, RDH12, PRPH2 (RDS), RHO, RPGR, SNRNP200, NR2E3, IMPDH1, CRX, HK1, IMPDH2, SNRNP200, PRPF3, or AGBL5 gene. The transgene is a NRL, RDH12, PRPH2 (RDS), RHO, RPGR, SNRNP200, NR2E3, IMPDH1, CRX, HK1, IMPDH2, SNRNP200, PRPF3, or AGBL5 gene. The variant AAV5 virion encapsidating the payload is administered to a subject. The subject is a human or non-human animal. The route of administration is an intravitreal route of administration. The intravitreal route of administration is intravitreal injection. Upon administration to the subject, the variant AAV5 virions encapsidating the therapeutic payload exhibit enhanced ocular tissue tropism as compared with wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of the retinitis pigmentosa is alleviated, or the subject is cured.

### Example 9

Treatment of Stargardt Disease with an AAV5-Derived Virion Encapsidating a Therapeutic Payload

This example describes treatment of Stargardt disease with a variant AAV5-derived virion having any one of the engineered ocular tissue-tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581 to 589 region, corresponding to residues 581 to 589 in the VP1 capsid polypeptide, and where the polynucleotide sequence encodes for a variant AAV5 Cap comprising a 581 to 589 region conferring retinal tissue targeting, retinal pigment epithelial cell targeting, or both. The 581 to 589 region has a sequence of any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. Each such variant is detargeted for vitreous humor and aqueous humor. The therapeutic payload is a guide RNA targeting an mRNA encoded for by a gene implicated in Stargardt disease, or the therapeutic payload is a transgene. The mRNA targeted by the guide RNA is an mRNA encoded for by an ABCA1, ABCA4, or CRB1 gene. The transgene is ABCA1, ABCA4, or CRB1. The variant AAV5 virion encapsidating the payload is administered to a subject. The subject is a human or non-human animal. The route of administration is an intravitreal route of administration. The intravitreal route of administration is intravitreal injection. Upon administration to the subject, the variant AAV5 virions encapsidating the therapeutic payload exhibit enhanced ocular tissue tropism as compared wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of Stargardt disease is alleviated, or the subject is cured.

### Example 10

Treatment of Uveal Melanoma with an AAV5-Derived Virion Encapsidating a Therapeutic Payload

This example describes treatment of uveal melanoma with a variant AAV5-derived virion having any one of the engineered ocular tissue-tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581 to 589 region, corresponding to residues 581 to 589 in the VP1 capsid polypeptide, and where the polynucleotide sequence encodes for a variant AAV5 Cap comprising a 581 to 589 region that confers retinal tissue targeting, retinal pigment epithelial cell targeting, or both. The 581 to 589 region has a sequence of any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. Each such variant is detargeted for vitreous humor and aqueous humor. The therapeutic payload is a guide RNA targeting an mRNA encoded for by a gene implicated in uveal melanoma, or the therapeutic payload is a transgene. The mRNA targeted by the guide RNA is an mRNA encoded for by a PTEN, BAP1, GNAQ, GNA11, DDEF1, SF3B1, EIF1AX, CDKN2A, p14ARF, a HERC2/OCA2 gene. The transgene is an anti gp100 protein. The variant AAV5 virion encapsidating the payload is administered to a subject. The subject is a human or non-human animal. The route of administration is an intravitreal route of administration. The intravitreal route of administration is intravitreal injection. Upon administration to the subject, the variant AAV5 virions encapsidating the therapeutic payload exhibit enhanced ocular tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of uveal melanoma is alleviated, a tumor size is reduced, or the subject is cured.

### Example 11

Treatment of Optical Neuropathy with an AAV5-Derived Virion Encapsidating a Therapeutic Payload

This example describes treatment of optical neuropathy with a variant AAV5-derived virion having any one of the engineered ocular tissue-tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581 to 589 region, corresponding to residues 581 to 589 in the VP1 capsid polypeptide, and where the polynucleotide sequence encodes for a variant AAV5 Cap comprising a 581 to 589 region that confers retinal tissue targeting, retinal pigment epithelial cell targeting, or both. The 581 to 589 region has a sequence of any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. Each such variant is detargeted for vitreous humor and aqueous humor. The therapeutic payload is a guide RNA targeting an mRNA encoded for by a gene implicated in optical neuropathy, or the therapeutic payload is a transgene. The mRNA targeted by the guide RNA is an mRNA encoded for by a Complex I, ND, OPA1, or RPE65 gene. The transgene is Complex I, ND, OPA1, or RPE65. The variant AAV5 virion encapsidating the payload is administered to a subject. The subject is a human or non-human animal. The route of administration is an intravitreal route of administration. The intravitreal route of administration is intravitreal injection. Upon administration to the subject, the variant AAV5 virions encapsidating the therapeutic payload exhibit enhanced ocular tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of ocular neuropathy is alleviated, or the subject is cured.

### Example 12

Treatment of Usher Syndrome with an AAV5-Derived Virion Encapsidating a Therapeutic Payload

This example describes treatment of Usher syndrome with a variant AAV5-derived virion having any one of the engineered ocular tissue-tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581 to 589 region, corresponding to residues 581 to 589 in the VP1 capsid polypeptide, and where the polynucleotide sequence encodes for a variant AAV5 Cap comprising a 581 to 589 region that confers retinal tissue targeting, retinal pigment epithelial cell targeting, or both. The 581 to 589 region has a sequence of any one of SEQ ID NO: 14-SEQ ID NO: 2013, SEQ ID NO: 2514-SEQ ID NO: 4513, SEQ ID NO: 2014-SEQ ID NO: 2513, or SEQ ID NO: 4514-SEQ ID NO: 5013. Each such variant is detargeted for vitreous humor and aqueous humor. The therapeutic payload is a guide RNA targeting an mRNA encoded for by a gene implicated in Usher syndrome, or the therapeutic payload is a transgene. The mRNA targeted by the guide RNA is an mRNA encoded for by a ADGRV1, CEP250, CLRN1, MYO7A, PROM1, USH1C, USH1G, or USH2A gene. The transgene is ADGRV1, CEP250, CLRN1, MYO7A, PROM1, USH1C, USH1G, or USH2A. The variant AAV5 virion encapsidating the payload is administered to a subject. The subject is a human or non-human animal. The route of administration is an intravitreal route of administration. The intravitreal route of administration is intravitreal injection. Upon administration to the subject, the variant AAV5 virions encapsidating the therapeutic payload exhibit enhanced ocular tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of Usher syndrome is alleviated, or the subject is cured.

### Example 13

### Singleplex Tertiary Screen to Validate Ocular Tissue-Tropic Engineered AAV Capsids

This example describes a tertiary screen, in singleplex, to identify ocular tissue-tropic engineered AAV capsids. Prospective tissue-tropic capsids identified from an in vivo secondary screen, through machine learning, or both, such as the retina tissue-tropic and RPE tissue-tropic capsids identified in EXAMPLE 4, are introduced into an in vivo tertiary screen, in singleplex, to confirm tissue-tropic behavior of the identified capsids when individually dosed in NHP. The tertiary screen includes capsids observed in the secondary screen in RPE, choroid, retina, or combinations thereof. The tertiary screen capsid library is injected into a non-human primate (NHP) by intravitreal injection. Following injection, ocular tissues, including RPE/choroid, retina, vitreous humor, aqueous humor, and optic nerve, are isolated from the NHP, and the barcoded sequences are quantified. RPE and choroid tissues are isolated and analyzed together. Prospective tissue-tropic capsids are compared to control, wild-type AAV to identify superior variant capsids of the present disclosure that target various compartments of the eye (e.g., RPE tissue-tropic capsids or retina tissue-tropic capsids).

While the invention has been particularly shown and described with reference to a preferred embodiment and various alternate embodiments, it is understood by persons skilled in the relevant art that various changes in form and details can be made therein without departing from the spirit and scope of the invention.

What is claimed is:

1. A viral protein (VP) capsid polypeptide comprising a 581 to 589 region corresponding to residues 581 to 589 of an AAV5 VP1 polypeptide,
wherein the 581 to 589 region comprises a sequence of SEQ ID NO: 5014 and further comprises at least one substitution relative to SEQ ID NO: 9; and
wherein the 581 to 589 region comprises one or more basic amino acid residues.

2. The VP capsid polypeptide of claim 1, wherein the 581 to 589 region comprises more basic amino acid residues than acidic amino acid residues.

3. The VP capsid polypeptide of claim 2, wherein the acidic amino acid residues are selected from the group consisting of D and E.

4. The VP capsid polypeptide of any one of claims 1-3, wherein the basic amino acid residues are selected from the group consisting of K, R, and H.

5. A viral protein (VP) capsid polypeptide comprising a 581 to 589 region corresponding to residues 581 to 589 of an AAV5 VP1 polypeptide, wherein the 581-589 region of the VP capsid polypeptide comprises a sequence of SEQ ID NO: 5014, wherein X¹, X², X³, X⁴, X⁵, or X⁶ of SEQ ID NO: 5014, or any combination thereof is K or R.

6. The VP capsid polypeptide of any one of claims 1-5, wherein X¹ of SEQ ID NO: 5014 is K or R.

7. The VP capsid polypeptide of claim 4, wherein the 581 to 589 region of the VP capsid polypeptide has a sequence of any one of SEQ ID NOs: 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2343, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2185, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, or 1555.

8. The VP capsid polypeptide of any one of claims 1-5, wherein X² of SEQ ID NO: 5014 is K or R.

9. The VP capsid polypeptide of claim 8, wherein the 581 to 589 region of the VP capsid polypeptide has a sequence of any one of SEQ ID NOs: 752, 2144, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 1308, 1230, 3960, 1378, 2994, 3080, 2589, 3681, 51, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 778, 87, 3891, 4456, 4040, 4496, 1982, 2001, 1478, 1189, 120, 4791, 2158, 121, 803, 804, 1463, 805, 122, 2139, 2290, 1760, 2465, 123, 130, 131, 132, 4933, 808, 2336, 2026, 2204, 809, 810, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 234, 4962, 2269, 843, 235, 844, 236, 237, 4930, 845, 846, 238, 847, 1295, 3601, 3877, 1930, 1420, 1678, 1460, 285, 1626, 299, 3809, 4271, 3844, 1436, 300, 1515, 1617, 3646, 4059, 1431, 1331, 310, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 1959, 1840, 3736, 1283, 3861, 1383, 3644, 3691, 4259, 1749, 1418, 1176, 4216, 1924, 1421, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 954, 955, 2437, 4139, 1482, 3602, 4434, 3721, 1592, 1825, 1698, 1519, 1352, 4202, 4409, 1977, 1917, 2382, 3675, 1833, 3974, 419, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 3879, 2698, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 1019, 2012, 1803, 3642, 497, 1322, 1261, 4027, 3478, 3763, 2959, 1293, 2462, 517, 3197, 1595, 3413, 1675, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1050, 3515, 2219, 1433, 1323, 4301, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2324, 605, 1368, 3827, 4103, 606, 607, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 4035, 2910, 3391, 2808, 3099, 2913, 3676, 2852, 3284, 2731, 1609, 2375, 2201, 3537, 3994, 1319, 4467, 1802, 1227, 1623, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 4879, 1841, 4234, 4924, 1154, 1314, 4096, 2023, 3815, 2005, 1964, 3439, 2833, 4287, 4509, 4505, 4267, 3697, 3272, 4112, 2967, 3651, 1991, 1805, 1628, 3705, or 2250.

10. The VP capsid polypeptide of any one of claims 1-5, wherein X³ of SEQ ID NO: 5014 is K or R.

11. The VP capsid polypeptide of claim 10, wherein the 581 to 589 region of the VP capsid polypeptide has a sequence of any one of SEQ ID NOs: 4177, 1289, 740, 741, 18, 745, 3477, 1548, 3051, 2982, 2877, 3036, 3426, 3073, 3169, 747, 3362, 2784, 21, 3192, 23, 1638, 748, 1703, 1836, 3660, 29, 750, 1640, 33, 34, 35, 36, 37, 38, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 3327, 3178, 3006, 3252, 2991, 3470, 2888, 2572, 3418, 2642, 2519, 3363, 40, 2633, 41, 42, 43, 755, 45, 756, 46, 48, 1952, 758, 49, 3122, 2551, 3718, 3484, 3577, 759, 1230, 3960, 1378, 2994, 3080, 2589, 3681, 760, 53, 3925, 55, 3654, 56, 61, 4086, 1740, 1660, 765, 766, 62, 1966, 63, 64, 768, 771, 65, 66, 67, 68, 3816, 2599, 2706, 74, 3865, 1549, 1361, 3882, 3713, 1942, 1312, 4501, 76, 77, 1560, 4289, 3757, 79, 3593, 4043, 1806, 3908, 4397, 1615, 4415, 3723, 2006, 3754, 1470, 4360, 3945, 2854, 3523, 3669, 82, 4065, 83, 1294, 1484, 3891, 4456, 4040, 4496, 1982, 2001, 1478, 1189, 1490, 1885, 1926, 89, 90, 91, 779, 1180, 92, 785, 786, 94, 95, 1831, 1867, 4458, 97, 1970, 1936, 1590, 100, 789, 790, 1298, 101, 1688, 791, 792, 2007, 103, 794, 795, 104, 105, 106, 1849, 108, 1579, 110, 1906, 113, 1444, 1344, 116, 802, 117, 118, 121, 803, 804, 1463, 805, 122, 124, 125, 127, 807, 132, 809, 810, 133, 1978, 812, 135, 136, 137, 138, 813, 140, 815, 1326, 141, 142, 143, 144, 145, 1634, 1302, 817, 146, 818, 147, 148, 149, 151, 152, 153, 154, 155, 819, 820, 157, 159, 160, 163, 164, 165, 166, 822, 167, 168, 169, 170, 171, 823, 824, 1651, 173, 1483, 174, 175, 825, 176, 177, 826, 827, 181, 828, 182, 829, 184, 185, 186, 187, 188, 189, 830, 191, 192, 193, 194, 195, 196, 1391, 197, 200, 201, 202, 203, 204, 205, 832, 833, 206, 834, 836, 1334, 837, 3901, 3312, 2649, 1301, 211, 212, 1450, 4386, 3028, 213, 1446, 214, 215, 1896, 1522, 3490, 838, 217, 218, 219, 1612, 220, 221, 222, 839, 223, 840, 224, 226, 227, 230, 231, 842, 232, 233, 844, 236, 237, 851, 852, 853, 1536, 240, 854, 242, 243, 244, 245, 246, 247, 249, 250, 251, 252, 253, 254, 255, 857, 256, 257, 258, 259, 858, 859, 260, 261, 860, 861, 862, 262, 264, 1241, 265, 266, 866, 1477, 267, 867, 869, 870, 271, 871, 872, 272, 1252, 273, 1591, 275, 1980, 276, 876, 877, 277, 278, 279, 1892, 1539, 880, 1295, 3601, 3877, 1930, 1420, 1678, 1460, 282, 1799, 283, 1626, 1374, 287, 1620, 1290, 1354, 888, 291, 1956, 889, 292, 4324, 293, 4138, 891, 3903, 297, 3829, 3809, 4271, 3844, 1436, 1515, 1617, 1961, 304, 306, 1589, 4370, 308, 309, 894, 3646, 4059, 1431, 1331, 312, 895, 1779, 313, 314, 315, 316, 896, 1558, 1552, 898, 321, 322, 899, 1958, 901, 325, 902, 326, 1491, 329, 330, 905, 4484, 1756, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 1888, 4248, 1557, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 3435, 3482, 3020, 2748, 2646, 2787, 2760, 2563, 3085, 3133, 3158, 3170, 2703, 3494, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 2979, 2542, 1762, 4472, 1472, 1529, 4057, 1976, 4507, 1583, 338, 911, 1711, 340, 912, 2013, 341, 4440, 917, 344, 918, 919, 921, 922, 347, 351, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 4145, 354, 925, 1782, 926, 927, 3610, 357, 359, 4224, 1918, 1959, 3736, 1770, 931, 4155, 364, 1279, 365, 1543, 367, 4049, 1633, 1722, 368, 369, 1495, 1566, 370, 1211, 1873, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 3741, 1938, 3709, 1727, 3893, 1280, 3622, 1784, 3834, 1752, 1983, 1743, 3979, 4461, 4113, 3817, 4232, 1340, 1489, 4214, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 377, 1395, 4453, 3940, 3778, 4374, 4210, 4009, 4264, 4339, 4368, 1738, 378, 1610, 4194, 4474, 4013, 4266, 1771, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 2002, 1657, 1663, 1336, 3894, 1554, 1494, 1405, 3576, 4314, 3695, 1851, 4362, 1815, 3845, 4511, 3656, 3638, 1406, 1596, 4454, 1443, 1661, 1359, 1734, 3650, 1910, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1680, 4441, 3628, 3725, 1945, 4206, 1588, 1439, 3794, 4447, 1838, 3776, 3658, 4064, 1377, 4011, 4272, 4503, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 3613, 3273, 3452, 3525, 1229, 4105, 3835, 383, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 1520, 1767, 1466, 1194, 4137, 1908, 1927, 1758, 3772, 3661, 4186, 4174, 1170, 3850, 1315, 1611, 1530, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 1716, 3961, 1506, 4221, 1376, 1683, 4276, 1445, 1721, 1632, 3607, 1328, 1455, 1913, 1497, 1317, 1951, 1778, 1379, 3768, 4225, 1668, 390, 391, 4406, 4341, 1480, 395, 1714, 1422, 396, 397, 3618, 4433, 1177, 3898, 398, 949, 950, 399, 951, 400, 1965, 4471, 952, 402, 2475, 403, 404, 4139, 1482, 3602, 4434, 3721, 1592, 1519, 1352, 4202, 4409, 1977, 1917, 407, 408, 1631, 1684, 409, 3889, 958, 959, 412, 960, 414, 415, 961, 1905, 416, 963, 417, 3675, 1833, 3974, 420, 421, 965, 966, 969, 970, 422, 1897, 423, 4133, 3830, 972, 424, 973, 427, 1708, 4141, 3749, 431, 1619, 975, 4044, 3590, 1944, 4126, 1923, 1706, 4400, 434, 1606, 979, 2604, 2895, 436, 3448, 1692, 983, 3862, 4055, 4358, 984, 985, 1664, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 3535, 1306, 4084, 3282, 1780, 987, 988, 990, 451, 452, 991, 992, 1728, 994, 995, 454, 1696, 3116, 1869, 456, 1386, 999, 3879, 2698, 1001, 461, 1447, 462, 464, 4425, 1645, 1231, 1468, 472, 473, 476, 1273, 1007, 1744, 1488, 1659, 477, 1604, 481, 4313, 482, 483, 1499, 484, 1016, 4073, 1777, 1458, 4274, 487, 1775, 4205, 1526, 1697, 1017, 2000, 489, 3917, 3616, 4311, 1800, 4473, 4085, 4401, 1310, 3783, 3765, 491, 1948, 3699, 1735, 3792, 4290, 495, 4090, 496, 2012, 1803, 3642, 1322, 498, 1021, 1022, 502, 1333, 503, 1023, 1890, 510, 1026, 3608, 1732, 511, 2397, 1864, 1816, 1531, 3636, 1699, 1027, 1029, 1366, 2112, 1261, 4027, 3478, 3763, 2959, 517, 3197, 1595, 3413, 1448, 518, 1675, 1797, 520, 1999, 3759, 1215, 4122, 1730, 524, 526, 3966, 3911, 529, 3289, 1821, 530, 531, 1874, 1038, 536, 537, 3767, 1929, 1041, 1042, 3683, 542, 1338, 4331, 4332, 4466, 2719, 3803, 4123, 4233, 1824, 3129, 3087, 3505, 4158, 4254, 1449, 3720, 4351, 4414, 1673, 3453, 3254, 1649, 546, 4240, 3580, 1046, 1047, 550, 2598, 551, 552, 3819, 1259, 4062, 2841, 1050, 3515, 4301, 1052, 1053, 3738, 1567, 1270, 3857, 1055, 1257, 1238, 3662, 561, 562, 1058, 4203, 1899, 3981, 1062, 1303, 4116, 567, 1946, 3620, 1514, 4002, 3679, 3964, 3707, 3666, 1348, 4273, 1804, 4497, 1401, 4438, 1895, 1264, 572, 1912, 3939, 4170, 4056, 1271, 1065, 1521, 1221, 1451, 1654, 3934, 4080, 4352, 1700, 3739, 1565, 1419, 1811, 3950, 1690, 1397, 3716, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 1723, 1070, 3750, 3633, 4070, 4104, 4162, 4235, 1863, 4346, 1881, 1814, 1572, 4443, 3761, 1346, 1587, 1072, 3592, 576, 1891, 4469, 1275, 1950, 1963, 4129, 1296, 1551, 3733, 4111, 3993, 4108, 3949, 1287, 1713, 1736, 1995, 1435, 1424, 1830, 1705, 1861, 1197, 1254, 4120, 1545, 4417, 1469, 1785, 1236, 4293, 3839, 3983, 4395, 1957, 3928, 4295, 3715, 3855, 3609, 1990, 4076, 4175, 3927, 1757, 1650, 4299, 1773, 2004, 1267, 1364, 1462, 1879, 4007, 3729, 3665, 3722, 1691, 1624, 1751, 1394, 3924, 3952, 580, 581, 2003, 1343, 1559, 1250, 1555, 4114, 1818, 1075, 1076, 1077, 1704, 4448, 1078, 1079, 1786, 585, 3354, 1832, 3432, 2891, 2505, 1903, 1081, 1082, 4459, 1083, 1563, 590, 591, 1085, 594, 1453, 2559, 2590, 2738, 4478, 4069, 1809, 1327, 4150, 3260, 3481, 3672, 3625, 2008, 4439, 3852, 3997, 1411, 599, 3847, 1268, 4050, 3377, 4424, 1496, 4143, 4493, 3492, 1962, 600, 601, 4017, 4477, 1794, 4263, 3072, 2009, 1754, 1087, 1088, 3888, 1368, 3827, 4103, 606, 1090, 1571, 1091, 1438, 1092, 1093, 612, 1094, 3701, 4389, 613, 1603, 1332, 1715, 4369, 1652, 1096, 617, 1541, 618, 1921, 1733, 1373, 1870, 619, 4218, 1798, 1100, 1702, 1101, 1667, 1384, 623, 3700, 1788, 1600, 624, 626, 2771, 627, 1339, 3875, 628, 629, 3070, 4860, 2557, 2795, 2906, 3118, 1614, 1947, 4479, 3766, 632, 1104, 1105, 633, 1813, 636, 3706, 1110, 640, 1111, 641, 642, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 3095, 1286, 3624, 4481, 1432, 3511, 2735, 4381, 3295, 1585, 1114, 1766, 3221, 2643, 2739, 3920, 644, 1116, 645, 646, 647, 1801, 4279, 3762, 1637, 1509, 1119, 2965, 2369, 2695, 3293, 650, 651, 1120, 652, 1121, 3264, 655, 658, 659, 4035, 2910, 3391, 2808, 3099, 2913, 3676, 2852, 3284, 2731, 1609, 3994, 1319, 4467, 1802, 1227, 1623, 662, 663, 664, 1124, 1125, 1128, 1129, 668, 669, 1130, 670, 1131, 1527, 1132, 671, 1772, 1135, 672, 1933, 1731, 1793, 675, 676, 3342, 1138, 1139, 677, 679, 1142, 1143, 681, 1145, 682, 685, 3822, 689, 690, 691, 1147, 1601, 3905, 694, 695, 1414, 1150, 1795, 1372, 1695, 1866, 4115, 1719, 698, 1920, 699, 700, 1544, 4305, 1636, 1393, 2010, 3810, 4452, 3677, 4227, 1277, 1400, 4402, 4157, 4197, 1337, 1253, 1532, 4028, 3989, 1493, 1854, 1902, 1510, 4182, 706, 707, 708, 4476, 1245, 714, 1841, 4234, 1314, 4096, 717, 1155, 4217, 1156, 723, 1157, 1627, 725, 1159, 1953, 1160, 1201, 727, 3787, 3747, 730, 1846, 3815, 2005, 1964, 1822, 1262, 4480, 1537, 1291, 1318, 4294, 1582, 734, 3439, 2833, 4287, 4509, 4505, 4267, 3697, 3272, 4112, 2967, 3651, 1628, 3705, 1195, 3859, 1164, 1165, 1166, 1745, 1973, 1168, 3807, 4307, 1454, or 1781.

12. The VP capsid polypeptide of any one of claims 1-5, wherein X⁴ of SEQ ID NO: 5014 is K or R.

13. The VP capsid polypeptide of claim 12, wherein the 581 to 589 region of the VP capsid polypeptide has a sequence of any one of SEQ ID NOs: 739, 4177, 1289, 15, 743, 3477, 3051, 2982, 2877, 746, 3036, 3426, 3073, 3169, 3362, 2784, 3192, 1638, 25, 1703, 1836, 3660, 30, 32, 752, 1653, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 1467, 3327, 3178, 3006, 3252, 2991, 3470, 2888, 2572, 3418, 2642, 2519, 3363, 2633, 757, 1952, 3122, 2551, 3718, 2216, 3484, 3577, 1308, 1230, 3960, 2994, 3080, 2589, 3681, 51, 761, 3925, 3654, 763, 764, 57, 4086, 1740, 767, 772, 2599, 2706, 70, 3865, 1549, 1361, 3882, 1942, 1312, 4501, 1413, 3593, 4043, 1806, 3908, 4397, 1615, 2006, 3945, 2854, 3523, 3669, 1356, 777, 4065, 84, 1484, 778, 87, 3891, 4456, 4040, 4496, 2001, 1478, 1189, 1490, 1885, 2270, 1180, 786, 4458, 1970, 1590, 98, 1298, 101, 1688, 794, 107, 1849, 797, 1579, 111, 112, 113, 1444, 801, 114, 115, 119, 120, 122, 1760, 123, 124, 806, 125, 126, 127, 128, 129, 130, 134, 811, 814, 139, 1326, 144, 816, 1302, 150, 152, 821, 160, 161, 162, 1651, 1483, 179, 180, 183, 185, 187, 1391, 198, 199, 831, 204, 833, 207, 3901, 3312, 2649, 1301, 4386, 3028, 1896, 1522, 3490, 216, 218, 1612, 225, 228, 229, 234, 239, 849, 850, 1536, 241, 246, 248, 855, 249, 252, 253, 261, 4966, 864, 263, 4749, 2296, 1241, 866, 268, 868, 269, 270, 873, 874, 1252, 273, 1591, 274, 1980, 878, 1539, 3601, 3877, 1930, 1420, 1678, 1460, 1799, 1374, 1290, 1354, 290, 2391, 4324, 294, 4138, 3903, 892, 3829, 298, 3844, 1436, 300, 1515, 1617, 1961, 303, 1589, 893, 4370, 3646, 4059, 1331, 1558, 1552, 320, 1958, 1491, 1635, 332, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3414, 3093, 2996, 3141, 3544, 3102, 2856, 2957, 3466, 3461, 2788, 2660, 3344, 3204, 2720, 3379, 2826, 2785, 3249, 3527, 3352, 2569, 4457, 2011, 3906, 1533, 1755, 1888, 4248, 1557, 3804, 1363, 1934, 1602, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 1852, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 1911, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 2662, 2875, 3223, 2878, 3285, 2757, 3057, 2951, 3519, 2579, 2853, 3387, 3401, 3493, 3393, 1608, 3365, 2885, 3574, 3081, 3007, 3568, 3152, 3367, 3358, 3468, 3290, 3550, 3132, 2726, 2879, 3190, 3309, 2613, 2765, 3454, 2851, 2881, 3232, 3458, 3326, 3353, 2927, 3212, 2950, 3376, 3208, 3062, 3612, 3195, 3385, 2686, 2602, 3569, 3123, 2727, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 2666, 3246, 3130, 3560, 3236, 2730, 3572, 1607, 3689, 2968, 1457, 1500, 1184, 3450, 2577, 2567, 2532, 3415, 2934, 2984, 2568, 4147, 3002, 2986, 910, 2979, 2542, 4472, 1472, 4057, 4507, 1711, 913, 916, 1594, 4440, 343, 918, 345, 348, 350, 1518, 3728, 3637, 3570, 3429, 2870, 2995, 4338, 3205, 2880, 3261, 1994, 3968, 4291, 4204, 4071, 352, 2820, 3912, 3287, 1218, 1190, 4145, 353, 355, 1782, 928, 3610, 358, 4224, 1918, 1959, 1840, 3736, 4155, 1543, 4049, 1633, 1495, 372, 1873, 1726, 1748, 4411, 4506, 4270, 4156, 3780, 4036, 4030, 4016, 1550, 3899, 1247, 1244, 4140, 3930, 4117, 4189, 4001, 3799, 3643, 3619, 4149, 1313, 3709, 1727, 3893, 1280, 3622, 1784, 3834, 1752, 3979, 4461, 4113, 3817, 4232, 3902, 1857, 1904, 1481, 1516, 3820, 3789, 4252, 4047, 4330, 4082, 4094, 3785, 3687, 4318, 4243, 3866, 1621, 4315, 1192, 1513, 1856, 3324, 2769, 2678, 3296, 375, 1395, 1584, 4093, 3940, 3778, 4374, 4210, 4009, 4264, 4368, 1610, 4194, 4474, 4013, 4266, 1759, 1922, 3663, 1642, 1185, 4356, 3932, 1233, 4212, 3858, 1198, 4192, 3790, 3712, 1969, 934, 1790, 1663, 1336, 3894, 1554, 1494, 1405, 3576, 4314, 3695, 3845, 4511, 3656, 1406, 1596, 4504, 1359, 1734, 3650, 1305, 1208, 1988, 4451, 1282, 4380, 1658, 1452, 1687, 3791, 4491, 4323, 4014, 1304, 1440, 4422, 3861, 1383, 3644, 3691, 4259, 1749, 4216, 1924, 1421, 1387, 3992, 4423, 4253, 1618, 1871, 1256, 3748, 1297, 1586, 1251, 1299, 1954, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 3627, 3777, 1371, 3685, 1325, 1940, 4482, 4258, 3812, 1581, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4153, 2707, 3730, 4426, 4180, 4041, 3923, 4444, 3851, 4429, 4361, 1441, 4394, 4302, 3758, 4510, 3742, 1666, 1407, 4187, 1188, 1724, 1646, 4280, 1613, 4006, 1984, 1351, 3653, 3833, 2561, 4442, 3900, 4298, 1203, 4441, 3628, 3725, 937, 4447, 3776, 3658, 4064, 1324, 1367, 3594, 3680, 1300, 1987, 1862, 3998, 1380, 4185, 3686, 3696, 4463, 1350, 4008, 4230, 3853, 4244, 4083, 1546, 3613, 3273, 3452, 3525, 4105, 3835, 1263, 4020, 4144, 1224, 1199, 3667, 2778, 4412, 4407, 1812, 1810, 3823, 1416, 4333, 3146, 4281, 4211, 1979, 1456, 1239, 4167, 3740, 1876, 1388, 1967, 1474, 1471, 4176, 1232, 1989, 4125, 1741, 3585, 1174, 1396, 4061, 4015, 1207, 3698, 3825, 3922, 4021, 4181, 1598, 1916, 1429, 4502, 4231, 1321, 4390, 3693, 3734, 3635, 1768, 4025, 3976, 3840, 3963, 4286, 1502, 3836, 4161, 3659, 3743, 4130, 3605, 1385, 1783, 4392, 3826, 1473, 1883, 1834, 3984, 4495, 3910, 4160, 3671, 4184, 4132, 4250, 1503, 1237, 1523, 1417, 4063, 1562, 3977, 1971, 1707, 3769, 4074, 1534, 4239, 4003, 3915, 1689, 1776, 1410, 1823, 1498, 1329, 386, 3936, 4054, 4260, 4359, 4131, 4297, 4357, 4022, 3770, 4046, 1222, 3953, 4228, 4226, 4277, 1520, 1767, 1466, 1194, 4137, 1908, 1927, 1758, 3772, 3661, 4186, 4174, 1170, 1859, 4183, 1408, 1403, 941, 4033, 3623, 1826, 3719, 1931, 3584, 4004, 3591, 3967, 4168, 3957, 3878, 4165, 4191, 3980, 4058, 4200, 4336, 4322, 3991, 4106, 1641, 1570, 3978, 4154, 1365, 4257, 3846, 4345, 4379, 3603, 1693, 3937, 3801, 4201, 1898, 1845, 1226, 1220, 3745, 3988, 3975, 4236, 4075, 4146, 4256, 4308, 4000, 4148, 3588, 3868, 4403, 3883, 4326, 4393, 3895, 4237, 3955, 3727, 4005, 4489, 3982, 4437, 3597, 4342, 3872, 3987, 4490, 3876, 3668, 4300, 4042, 3648, 3996, 3800, 4367, 4099, 1807, 3887, 4428, 1884, 1682, 4364, 4306, 3869, 3600, 3914, 1968, 1670, 3811, 1865, 3961, 1506, 4221, 1376, 1379, 3768, 4225, 945, 394, 1714, 1422, 3618, 4433, 1965, 4471, 954, 4139, 1482, 3721, 1825, 1698, 1519, 1352, 4202, 4409, 1977, 1917, 405, 406, 3889, 413, 962, 418, 3675, 3974, 964, 420, 967, 968, 1897, 1272, 4133, 425, 4141, 430, 3749, 4044, 3590, 1944, 4126, 977, 4400, 1606, 980, 2604, 2895, 3448, 4563, 1692, 4055, 4358, 1664, 4309, 4045, 3854, 3682, 2688, 4366, 3425, 3231, 3649, 2759, 2724, 3873, 3755, 1216, 2754, 2606, 3471, 3234, 2565, 2803, 2562, 2684, 3411, 3405, 2690, 1255, 3985, 3535, 1306, 4084, 3282, 447, 1353, 1780, 449, 1485, 453, 1728, 454, 1696, 455, 3116, 1869, 457, 1386, 3879, 2698, 459, 1447, 4425, 1645, 467, 1488, 478, 1604, 1011, 4313, 4073, 4274, 1775, 2000, 2480, 1882, 1817, 1880, 1800, 4473, 3783, 3870, 1948, 3699, 1735, 3792, 4290, 492, 4090, 1019, 1803, 3642, 497, 500, 1333, 1890, 1024, 3608, 1864, 1531, 3636, 1699, 1028, 1366, 1030, 4027, 3478, 3763, 2959, 1293, 517, 3197, 1595, 3413, 2238, 1031, 1448, 1032, 1675, 1797, 3759, 1215, 4122, 3966, 528, 3911, 3289, 1821, 1874, 1037, 536, 1039, 3767, 3683, 540, 541, 1338, 1616, 1901, 1517, 2719, 3803, 4123, 4233, 3129, 3087, 3505, 4158, 1044, 3720, 4351, 4414, 1673, 3453, 3254, 1649, 543, 4240, 3580, 2598, 4062, 2841, 1049, 3515, 1433, 1323, 4301, 1051, 3738, 1270, 3857, 1056, 3662, 1057, 3981, 1063, 568, 570, 3620, 1064, 4002, 3964, 3707, 3666, 1348, 4273, 4497, 1401, 4438, 1895, 1912, 3939, 4170, 4056, 1221, 1654, 1067, 3934, 4080, 4352, 574, 3739, 1565, 3950, 1397, 3716, 1569, 1465, 4268, 1183, 3726, 3881, 4134, 4347, 4012, 3938, 4066, 4198, 4416, 4164, 3630, 4325, 4375, 4371, 4432, 4215, 1753, 3944, 1409, 4195, 4455, 4512, 3750, 3633, 4070, 4104, 4162, 4235, 4346, 1572, 4443, 3761, 1346, 3592, 1891, 4469, 1275, 577, 1963, 4129, 1296, 1360, 3733, 4111, 3993, 4108, 3949, 1556, 1435, 1424, 1830, 1197, 1254, 4120, 1545, 4417, 1469, 1292, 4293, 1827, 3839, 3983, 4395, 4295, 3715, 3855, 3609, 4076, 4175, 3927, 1650, 4299, 1773, 2004, 1267, 1364, 4007, 3729, 3665, 3722, 1712, 1751, 3924, 3952, 1343, 4114, 1704, 4448, 1786, 3354, 586, 3432, 2891, 587, 1903, 2077, 1080, 1563, 2274, 595, 2559, 2590, 2738, 4478, 4069, 1327, 4150, 3260, 3481, 3672, 3625, 2008, 4439, 3852, 3997, 1411, 1268, 4050, 3377, 4424, 1496, 4143, 4493, 3492, 1962, 1412, 4017, 4477, 1794, 4263, 3072, 2199, 3888, 604, 605, 1368, 3827, 4103, 607, 1571, 1438, 3701, 4389, 4897, 1603, 2154, 1715, 4369, 1652, 1733, 4218, 1798, 621, 1667, 1384, 3700, 1788, 1600, 1102, 1423, 2771, 1339, 3070, 2557, 2795, 2906, 3299, 3118, 4779, 2028, 1614, 1947, 630, 4479, 3766, 1106, 634, 1108, 635, 638, 3706, 3735, 3390, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 1269, 3095, 1286, 3624, 4481, 3511, 2735, 4381, 3295, 1585, 3221, 2643, 2739, 3920, 1801, 4279, 2502, 1117, 2965, 2695, 3293, 3264, 2076, 657, 2910, 3391, 2808, 3099, 2913, 3676, 2852, 3284, 2731, 1609, 3537, 3994, 1319, 4467, 1802, 1227, 1623, 667, 1133, 1134, 673, 1933, 1731, 1793, 3342, 683, 1146, 687, 3822, 1601, 692, 3905, 1414, 696, 1695, 4115, 701, 1544, 702, 703, 4031, 2010, 3810, 4452, 3677, 4227, 4402, 4157, 4197, 1337, 1253, 1532, 1493, 1854, 1510, 705, 1153, 4476, 1375, 712, 1245, 715, 4234, 1154, 1314, 4096, 720, 1311, 721, 722, 4217, 726, 3815, 2005, 1964, 1262, 4480, 4294, 1582, 3439, 2833, 4287, 4509, 3272, 4112, 2967, 3651, 1991, 1805, 1628, 3705, 1195, 3859, 3807, 4307, 1454, or 1781.

14. The VP capsid polypeptide of any one of claims 1-5, wherein X⁵ of SEQ ID NO: 5014 is K or R.

15. The VP capsid polypeptide of claim 14, wherein the 581 to 589 region of the VP capsid polypeptide has a sequence of any one of SEQ ID NOs: 2498, 4177, 742, 16, 17, 18, 745, 3477, 1548, 2982, 3426, 3073, 3169, 747, 3362, 4942, 23, 28, 2038, 3660, 750, 31, 32, 1640, 34, 37, 4925, 1205, 4288, 4053, 3263, 2553, 3947, 2710, 2608, 3467, 2796, 2961, 2529, 3320, 2674, 2635, 3446, 3053, 3688, 4320, 3708, 2807, 3562, 3274, 3374, 3013, 3416, 2981, 3065, 2777, 3469, 2977, 2638, 2972, 3059, 2574, 3380, 4262, 3167, 3368, 1225, 3178, 3006, 3252, 2991, 2642, 2519, 3363, 2633, 42, 755, 44, 756, 46, 1952, 758, 2410, 2551, 3718, 1308, 1230, 3960, 1378, 2589, 3681, 760, 53, 4951, 4591, 762, 3925, 55, 3654, 763, 60, 61, 4086, 1740, 766, 1966, 63, 2041, 64, 769, 770, 2384, 771, 65, 772, 2197, 68, 3816, 69, 2599, 70, 3865, 1549, 3882, 75, 4501, 76, 774, 1413, 776, 3757, 3593, 4043, 4397, 4415, 3754, 1470, 4360, 3945, 2854, 3523, 3669, 1356, 81, 82, 4065, 83, 84, 1294, 87, 3891, 4456, 4040, 1189, 1490, 90, 92, 782, 94, 1831, 1867, 4458, 1936, 100, 792, 2007, 4556, 795, 104, 106, 1849, 2509, 110, 1906, 798, 2359, 2461, 4864, 1344, 116, 120, 121, 803, 1463, 805, 1760, 123, 124, 806, 127, 2085, 130, 132, 809, 1978, 134, 813, 139, 815, 1326, 141, 142, 145, 4816, 1302, 817, 146, 818, 148, 149, 4546, 150, 153, 156, 157, 163, 164, 166, 169, 823, 824, 1483, 175, 177, 178, 179, 826, 827, 182, 183, 829, 184, 185, 190, 191, 192, 2361, 195, 1391, 197, 2223, 201, 202, 4775, 1334, 837, 3901, 3312, 212, 4386, 3028, 213, 1446, 214, 215, 1896, 3490, 217, 219, 223, 225, 2088, 4542, 226, 227, 230, 843, 844, 237, 845, 239, 849, 852, 1536, 854, 244, 245, 248, 855, 251, 253, 255, 257, 858, 860, 262, 863, 4535, 865, 264, 1241, 265, 1477, 267, 867, 1252, 273, 275, 1980, 277, 281, 1892, 879, 1539, 881, 1295, 282, 285, 1626, 2082, 1374, 1620, 4564, 289, 887, 888, 889, 4324, 293, 4138, 296, 3903, 892, 3829, 298, 301, 302, 1589, 4370, 2342, 309, 4059, 310, 1779, 1558, 1552, 317, 320, 898, 321, 2016, 2134, 324, 325, 327, 1635, 4484, 1756, 4609, 332, 4635, 3069, 2637, 3025, 2782, 3247, 3350, 2692, 2586, 3214, 3163, 2541, 3090, 3097, 3462, 3037, 3419, 2780, 2931, 1276, 2789, 3228, 2657, 3189, 2975, 3346, 3067, 2728, 2955, 3408, 3027, 3094, 3203, 2988, 2751, 2953, 2566, 2578, 2901, 2837, 2822, 2745, 3473, 3530, 3040, 2597, 3509, 3522, 3161, 2926, 3209, 3105, 3400, 2630, 2905, 2671, 2818, 2806, 2534, 2773, 2623, 3491, 3243, 3136, 2693, 3164, 2679, 3510, 3110, 2672, 3442, 2929, 3184, 3488, 3102, 3466, 3461, 2788, 2660, 3344, 2720, 3379, 2826, 3249, 3527, 4460, 3352, 4319, 3906, 4248, 3804, 1363, 1404, 4349, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3782, 3629, 3578, 3885, 4081, 1764, 1852, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 4222, 1504, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3909, 4269, 3019, 1415, 3020, 2748, 2646, 2787, 2760, 3158, 3170, 2703, 3494, 3404, 2675, 2749, 3341, 3355, 2594, 3495, 2884, 3516, 3042, 2737, 2923, 3563, 2855, 3476, 2611, 2937, 3383, 2875, 3223, 2878, 1808, 2951, 3519, 2579, 2853, 3387, 3401, 3007, 3568, 3152, 3367, 3358, 336, 3290, 3550, 3309, 2613, 2765, 1355, 2881, 3232, 3458, 3326, 3353, 2927, 3212, 2950, 3376, 3208, 3062, 3612, 3195, 3385, 2686, 1622, 3361, 3022, 3427, 3107, 3140, 3371, 3276, 2831, 3501, 2924, 3366, 3199, 3101, 3321, 3499, 2917, 2683, 1464, 3560, 3236, 2730, 3572, 1607, 909, 2577, 2567, 2532, 3415, 2934, 2984, 2568, 3002, 2338, 4472, 1472, 1529, 4507, 1583, 340, 912, 2013, 913, 2277, 1594, 4440, 917, 343, 344, 345, 346, 350, 351, 4498, 3637, 3570, 3429, 4091, 4338, 3205, 2880, 3261, 3968, 4291, 4204, 4034, 2820, 3912, 3287, 1218, 1190, 4145, 354, 3610, 4224, 1840, 3736, 1770, 4155, 932, 363, 1279, 2156, 1722, 368, 1566, 370, 4967, 372, 1211, 2494, 1726, 1748, 1191, 4039, 4411, 4506, 4270, 4156, 3780, 1550, 3899, 1181, 3919, 1639, 3664, 4189, 4001, 3799, 1240, 1686, 1479, 1307, 1575, 4296, 1665, 3871, 1605, 4408, 4149, 3741, 3893, 1280, 1983, 3979, 3817, 1489, 4214, 3902, 1857, 1904, 3820, 3789, 4252, 4330, 4082, 3785, 3687, 4243, 3704, 4172, 4315, 1192, 2769, 2678, 3296, 2343, 4093, 4453, 3940, 3778, 4374, 4339, 4368, 4194, 4474, 4013, 1771, 1759, 3860, 3832, 1381, 4356, 1233, 4212, 3858, 1198, 3611, 1919, 1819, 3790, 1969, 4247, 1790, 3894, 1554, 3576, 4314, 1851, 1850, 4511, 3656, 3638, 1406, 1443, 1661, 4504, 3831, 4435, 1208, 1988, 3849, 1835, 4380, 4024, 4491, 4323, 4014, 1511, 1981, 1676, 1547, 1383, 3644, 3691, 4259, 1176, 4216, 1924, 1387, 4423, 1256, 4119, 1251, 3990, 4029, 4097, 3890, 3796, 4343, 1202, 4413, 4384, 3808, 3892, 3926, 3756, 4405, 4109, 1210, 4492, 3969, 4436, 3175, 4128, 2970, 1228, 3534, 3437, 3784, 4470, 4010, 4334, 3821, 4171, 3824, 3631, 4494, 1320, 1204, 1223, 1284, 1685, 3760, 3753, 1178, 3884, 2783, 4038, 3545, 2916, 3112, 2834, 3880, 3673, 4102, 4100, 4327, 3634, 3617, 4445, 4303, 1370, 3838, 1187, 3615, 3746, 3935, 1941, 4419, 1171, 3670, 3777, 936, 3685, 1325, 1940, 4482, 4258, 3812, 4124, 4508, 4500, 3652, 3972, 1630, 4421, 1172, 4026, 2740, 3125, 3546, 4513, 4340, 1763, 1487, 3589, 3907, 4041, 3851, 4077, 4187, 1193, 1196, 1724, 1717, 1200, 1975, 1613, 4006, 3653, 3833, 1593, 4442, 3900, 4388, 1820, 4317, 4249, 3628, 3794, 4447, 3776, 3658, 1377, 4011, 4272, 3594, 4185, 3686, 3696, 1350, 4230, 3853, 4244, 1909, 3897, 1546, 3273, 3452, 3525, 4105, 3835, 383, 1263, 1492, 3856, 3586, 1175, 4144, 1224, 1199, 3667, 2778, 4412, 1507, 3146, 4281, 1179, 3843, 3740, 1388, 1474, 1342, 1471, 4176, 1741, 1508, 4378, 1212, 4015, 1207, 3698, 3825, 4231, 3806, 3896, 3963, 4286, 3743, 4130, 3605, 4173, 4265, 1246, 1737, 3826, 1473, 1992, 1883, 1656, 3671, 4063, 1847, 4239, 3915, 1265, 1498, 1329, 3874, 4261, 1791, 3579, 4359, 4131, 4297, 4357, 4046, 3788, 4169, 1925, 4228, 4101, 1889, 1369, 4088, 4019, 1194, 3772, 3850, 1315, 1530, 1859, 4183, 1408, 4033, 3623, 1928, 1459, 1765, 4004, 3591, 3967, 4168, 3957, 3878, 3980, 3958, 4051, 3798, 4032, 3991, 4106, 1641, 4464, 3813, 4257, 3846, 4345, 1787, 1214, 4483, 4201, 1220, 3867, 3640, 3916, 1209, 4236, 4075, 4146, 4256, 4308, 4000, 4148, 3588, 3883, 4326, 4092, 4283, 1553, 3805, 4151, 1281, 1681, 4489, 3982, 4437, 3597, 4342, 3987, 3583, 3647, 389, 4304, 3793, 1937, 3996, 3800, 4367, 4099, 1390, 4364, 4306, 1278, 1672, 1542, 1915, 3600, 3948, 3811, 1362, 4276, 1445, 1721, 1632, 3607, 1317, 1778, 1668, 391, 4406, 4341, 1480, 393, 394, 395, 946, 3618, 4433, 1177, 947, 950, 400, 4471, 401, 402, 954, 3602, 4434, 3721, 1825, 4202, 4409, 406, 1631, 1684, 3889, 410, 412, 2352, 1905, 417, 418, 3675, 1833, 3974, 419, 964, 420, 421, 969, 4733, 1272, 971, 423, 3830, 973, 426, 1708, 428, 4141, 3749, 1619, 975, 4044, 3590, 1706, 4400, 979, 2895, 3448, 438, 982, 2433, 983, 442, 3862, 2435, 443, 4309, 4045, 3854, 3682, 444, 445, 4366, 3425, 3231, 3649, 2759, 2724, 3873, 3599, 3775, 3471, 3234, 2565, 2803, 2562, 2684, 3411, 3405, 2690, 3694, 446, 3985, 3535, 4084, 3282, 988, 2107, 990, 451, 452, 453, 992, 993, 1696, 996, 3116, 1386, 999, 458, 3879, 460, 461, 462, 2339, 1003, 4425, 1645, 1004, 466, 2328, 468, 1231, 1468, 2496, 473, 474, 2108, 1273, 1007, 1744, 1659, 478, 1009, 1012, 4313, 482, 1499, 484, 2513, 2205, 4592, 1458, 4274, 2122, 4905, 4205, 2000, 488, 1894, 3917, 3616, 4311, 4085, 4401, 1310, 3783, 1538, 3870, 1948, 3699, 1735, 3792, 2202, 4090, 2378, 2012, 3642, 1020, 2302, 4665, 4781, 500, 1022, 1023, 4686, 505, 507, 508, 509, 1025, 1732, 1816, 3636, 1028, 516, 1366, 4572, 2489, 1261, 4027, 3763, 2959, 1293, 3197, 1448, 1032, 519, 2420, 522, 3759, 1215, 4122, 1730, 524, 526, 3966, 1035, 527, 528, 529, 530, 531, 1036, 532, 533, 536, 537, 3767, 1929, 1041, 3683, 1043, 4906, 542, 1338, 1796, 4331, 4332, 4466, 3803, 3087, 3505, 1449, 1044, 4414, 3453, 3254, 544, 546, 1045, 547, 4240, 3580, 4680, 549, 550, 2598, 551, 3819, 1259, 4062, 554, 1049, 1050, 1433, 555, 1052, 1053, 3738, 3857, 2089, 1056, 2193, 1257, 1238, 3662, 1059, 1060, 4203, 3981, 1303, 4116, 567, 569, 3620, 4002, 3679, 3707, 4497, 4438, 1264, 3939, 4170, 4056, 1271, 1451, 1654, 3934, 4080, 4352, 574, 3739, 1565, 1419, 3950, 3716, 1465, 3726, 3881, 4134, 4347, 4012, 3938, 4066, 4164, 3630, 4229, 4292, 4215, 1729, 4223, 4455, 4512, 3633, 4070, 4104, 4162, 4443, 3761, 1587, 3592, 1891, 4469, 1963, 4129, 1360, 3949, 1861, 4120, 1292, 4293, 3839, 3983, 3928, 4295, 3715, 3855, 3609, 1990, 4007, 3722, 1624, 3924, 3952, 580, 2003, 1343, 582, 4114, 1818, 1704, 4448, 584, 1078, 1786, 585, 1832, 586, 2891, 587, 4936, 1082, 4459, 2113, 590, 2308, 593, 1858, 597, 2738, 4150, 3260, 3481, 4439, 1268, 4050, 3377, 4424, 3492, 1412, 4477, 4263, 3072, 2009, 3888, 3827, 4103, 1438, 609, 610, 611, 3701, 4389, 613, 4369, 617, 1921, 1373, 1870, 4218, 1099, 1702, 4801, 623, 3700, 1788, 625, 4878, 1423, 1339, 3875, 628, 2557, 2906, 2029, 4876, 1103, 631, 4479, 3766, 632, 1104, 1105, 634, 4651, 1108, 1813, 636, 638, 641, 4581, 1112, 2164, 3735, 3390, 2558, 3314, 3086, 3029, 2956, 3517, 2651, 2914, 2680, 2632, 3121, 3100, 3528, 3526, 3084, 4220, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 4398, 1269, 3624, 4481, 1432, 3295, 1568, 1766, 2643, 2739, 4760, 1115, 3920, 644, 645, 646, 4279, 3762, 1637, 1509, 2200, 1117, 4780, 2965, 2695, 650, 651, 653, 654, 655, 658, 4035, 2910, 3391, 2808, 3676, 2852, 3284, 3537, 1319, 4467, 1802, 1227, 660, 4812, 2364, 1124, 1125, 665, 4917, 1127, 1129, 1132, 1133, 2236, 673, 4557, 1136, 1139, 678, 4737, 4531, 679, 680, 1144, 681, 4525, 2415, 685, 2445, 3822, 690, 691, 1601, 4621, 3905, 695, 1149, 696, 2027, 2160, 1795, 1372, 4115, 2194, 1920, 4031, 1393, 1277, 1400, 4402, 4157, 4028, 704, 1902, 1510, 4182, 1153, 4476, 2053, 710, 1375, 715, 4096, 717, 1155, 719, 1311, 722, 4217, 1156, 723, 1157, 1627, 4566, 1158, 724, 725, 1953, 1201, 3787, 3747, 1846, 3815, 1822, 1537, 1318, 4294, 733, 734, 3439, 2833, 4505, 4267, 3697, 4112, 2967, 3651, 1805, 1163, 3859, 2275, 737, 1167, 1745, 3807, or 4307.

16. The VP capsid polypeptide of any one of claims 1-5, wherein X⁶ of SEQ ID NO: 5014 is K or R.

17. The VP capsid polypeptide of claim 16, wherein the 581 to 589 region of the VP capsid polypeptide has a sequence of any one of SEQ ID NOs: 738, 4177, 1289, 740, 14, 743, 744, 17, 3477, 1548, 3051, 2982, 2877, 746, 19, 3036, 3426, 3073, 3169, 747, 3362, 2784, 21, 3192, 22, 1638, 748, 24, 749, 27, 1703, 1836, 28, 3660, 29, 750, 1640, 33, 35, 36, 1653, 1205, 3795, 753, 4288, 4219, 2770, 2962, 4241, 4107, 2718, 3317, 4391, 3263, 2553, 3947, 2710, 2608, 3467, 2796, 2961, 2529, 3320, 2674, 2635, 3446, 3053, 3250, 3688, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 1467, 3327, 3178, 3006, 3252, 2991, 3470, 2888, 2572, 3418, 2642, 2519, 3363, 40, 2633, 44, 45, 756, 47, 48, 1952, 3122, 2551, 3718, 3484, 3577, 1308, 3960, 1378, 2994, 3080, 2589, 3681, 51, 760, 762, 3925, 3654, 763, 59, 60, 4086, 1740, 1660, 62, 1966, 63, 2438, 768, 66, 67, 68, 3816, 2599, 2706, 72, 73, 74, 3865, 1549, 1361, 3882, 773, 3713, 1942, 1312, 4501, 76, 1413, 775, 1560, 4289, 3757, 3593, 3908, 4397, 4415, 3723, 3754, 4360, 3945, 2854, 3523, 3669, 1356, 4065, 1294, 1484, 85, 86, 3891, 4456, 4040, 4496, 1982, 2001, 1478, 1189, 1490, 1885, 1926, 89, 780, 1180, 92, 781, 782, 783, 786, 94, 95, 788, 1831, 1867, 96, 4458, 97, 1970, 1936, 1590, 98, 99, 789, 790, 1298, 1688, 791, 792, 2007, 102, 103, 104, 105, 107, 108, 1579, 1906, 111, 798, 112, 799, 800, 1444, 801, 114, 115, 1344, 116, 802, 117, 118, 119, 121, 803, 1463, 1760, 806, 126, 128, 807, 129, 131, 808, 133, 1978, 811, 812, 135, 136, 137, 138, 814, 140, 1326, 141, 142, 143, 2344, 816, 1634, 1302, 817, 818, 147, 148, 149, 150, 151, 154, 155, 819, 820, 156, 157, 158, 161, 162, 165, 822, 168, 169, 170, 823, 172, 1651, 173, 1483, 174, 825, 176, 178, 180, 827, 181, 828, 4911, 184, 186, 188, 189, 830, 192, 193, 194, 196, 1391, 198, 199, 200, 201, 202, 203, 205, 832, 206, 834, 207, 208, 209, 210, 835, 836, 1334, 3901, 3312, 2649, 1450, 4386, 3028, 1446, 1522, 3490, 217, 219, 1612, 220, 221, 222, 839, 223, 840, 224, 225, 228, 229, 230, 231, 842, 232, 233, 234, 238, 847, 239, 850, 851, 853, 1536, 240, 241, 242, 243, 244, 245, 247, 250, 251, 254, 255, 857, 256, 258, 259, 859, 260, 861, 862, 864, 263, 2318, 865, 2326, 264, 1241, 266, 1477, 867, 268, 868, 269, 270, 869, 870, 271, 871, 872, 272, 873, 1252, 1591, 875, 274, 275, 276, 876, 877, 277, 278, 280, 1892, 1539, 881, 3601, 3877, 1460, 282, 1799, 284, 1626, 286, 1374, 1620, 1290, 1354, 887, 1956, 889, 292, 4324, 4138, 891, 296, 3903, 297, 3829, 3809, 4271, 3844, 1436, 300, 1617, 1961, 302, 304, 305, 2147, 1589, 4370, 307, 3646, 4059, 1431, 1331, 312, 1779, 313, 896, 1558, 1552, 318, 319, 322, 1958, 323, 324, 901, 1491, 328, 329, 4484, 907, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3414, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 4248, 1557, 3804, 1602, 4188, 1330, 4349, 3337, 3533, 2629, 3038, 2539, 3567, 3291, 3348, 3115, 3359, 2993, 3079, 3506, 2527, 2963, 2938, 2516, 3113, 2639, 3143, 3044, 2747, 3154, 3280, 3039, 2743, 2902, 3056, 2554, 2522, 3103, 3210, 3434, 2656, 4251, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 2869, 2610, 3298, 2775, 3108, 2603, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 2584, 2712, 3213, 3451, 2634, 3150, 2810, 3303, 2573, 3460, 2615, 3394, 2828, 3329, 4377, 4499, 3555, 2974, 3629, 3578, 3885, 2817, 3248, 2954, 3334, 3571, 3345, 3551, 3384, 3422, 2530, 2973, 4418, 4081, 3951, 1764, 1671, 1843, 3449, 3185, 3399, 2786, 3055, 3472, 3397, 2601, 3564, 3277, 3548, 3179, 3407, 3841, 3347, 3142, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3120, 3286, 2843, 3465, 3946, 2682, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 4269, 3487, 3489, 3019, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1464, 1886, 2666, 3246, 3130, 3560, 3236, 2730, 3572, 3689, 2968, 1457, 3450, 2577, 2567, 2532, 3415, 2934, 2984, 2568, 4147, 3002, 2986, 910, 2979, 2542, 1762, 4472, 1529, 4057, 1976, 4507, 1583, 1711, 2013, 1594, 4440, 344, 920, 921, 4498, 3728, 3637, 3570, 3429, 4091, 2870, 2995, 4338, 3205, 2880, 3261, 3968, 4291, 4204, 4071, 4034, 1742, 2820, 3912, 3287, 1218, 1190, 4145, 354, 1782, 927, 3610, 356, 4224, 1918, 1959, 1840, 3736, 1770, 929, 930, 931, 4155, 360, 361, 362, 364, 1279, 365, 1543, 366, 367, 4049, 1633, 1722, 369, 1495, 1566, 373, 1211, 1726, 1442, 1748, 4142, 1434, 1358, 1191, 3732, 4110, 4039, 4506, 4156, 4036, 4030, 4016, 3899, 1247, 1855, 3919, 1244, 4140, 3930, 4117, 4189, 4001, 3643, 3619, 1240, 1575, 3871, 4135, 4408, 3604, 1274, 4149, 3741, 1938, 3709, 3622, 3834, 1983, 1743, 3979, 4461, 4113, 3817, 4232, 4214, 3902, 1219, 1486, 1512, 1516, 4047, 4082, 4094, 1574, 1248, 4078, 1872, 1426, 3687, 4318, 4243, 3866, 3704, 4172, 1856, 3324, 2769, 2678, 1561, 3296, 377, 1395, 1584, 4093, 4453, 4210, 4009, 4264, 4339, 1738, 4474, 4013, 4266, 1771, 1759, 1922, 3860, 3832, 1349, 1381, 3663, 1185, 3932, 3858, 1198, 4192, 3611, 1919, 3790, 3712, 4095, 1887, 4468, 1720, 4247, 934, 1790, 2002, 1657, 1336, 3894, 3576, 4314, 3695, 1850, 4362, 1815, 3638, 4454, 1661, 4504, 1359, 3650, 1305, 1648, 3831, 4435, 4451, 1993, 3849, 1718, 4068, 1835, 1425, 1428, 4380, 1658, 1452, 4024, 4072, 3791, 4491, 4323, 4014, 1304, 1511, 1981, 1676, 4422, 1283, 3861, 3644, 4259, 1418, 1176, 4216, 1421, 3992, 4423, 4253, 3748, 4119, 1297, 1954, 4450, 3711, 4344, 1578, 4384, 3892, 3674, 3595, 4405, 3973, 4109, 4159, 3744, 4387, 4275, 4492, 4365, 4118, 4278, 3751, 3678, 4353, 3175, 2970, 1228, 3534, 3437, 3406, 3641, 4470, 3941, 4348, 3587, 3818, 4350, 3821, 3929, 3824, 4465, 1398, 3828, 1284, 3773, 4209, 3014, 3884, 2783, 4038, 3545, 2916, 3112, 2834, 3880, 4399, 4048, 3752, 4207, 1235, 3943, 4355, 4087, 4100, 4163, 4486, 4178, 1643, 4152, 4445, 4303, 4284, 1213, 4382, 1399, 3714, 4487, 3615, 4018, 4420, 4121, 3724, 4282, 4310, 4488, 4127, 3670, 4052, 3627, 3777, 1371, 3685, 4258, 3812, 1581, 4124, 4508, 4500, 3652, 3972, 1528, 3717, 2740, 3125, 3546, 3842, 4404, 3690, 3962, 4136, 3589, 3907, 4153, 2707, 4180, 3923, 4444, 4361, 4394, 3758, 4510, 3742, 1710, 1407, 4187, 1193, 1188, 1196, 1724, 1717, 1200, 1997, 1701, 4280, 4006, 1984, 3833, 2561, 4442, 3900, 4298, 1761, 4388, 1597, 4317, 4249, 1341, 1203, 1680, 4441, 3628, 3725, 1945, 937, 4206, 1588, 1439, 3794, 4447, 3776, 3658, 4064, 4011, 4272, 4503, 1324, 3594, 3680, 1300, 1750, 3913, 3774, 1877, 1987, 3998, 3686, 3696, 4463, 3853, 4244, 4083, 1909, 3897, 1546, 3613, 3273, 3452, 3525, 1229, 3835, 1234, 3970, 3856, 3586, 1175, 4321, 2778, 4412, 4407, 3823, 1427, 1507, 1878, 4333, 3146, 4281, 4211, 3843, 1677, 1524, 1525, 1974, 1239, 4167, 3740, 1342, 1471, 4176, 1232, 1989, 4125, 1741, 1580, 4060, 1576, 1212, 3921, 1174, 4061, 4021, 3693, 3734, 1382, 3684, 3806, 3635, 3976, 3840, 4286, 3836, 4161, 3659, 3605, 4392, 4328, 1316, 4173, 4265, 1246, 1629, 1829, 3826, 1992, 1656, 1914, 1839, 3984, 3910, 4160, 4184, 4132, 4250, 1792, 3769, 1534, 4003, 1689, 1599, 1265, 1410, 1823, 1498, 1329, 386, 940, 1655, 3874, 4261, 3626, 3579, 1644, 3936, 4054, 4260, 4297, 4357, 4022, 4046, 1222, 1996, 3788, 3953, 4226, 4277, 4101, 3797, 4019, 3771, 3632, 1402, 4137, 3772, 3661, 4186, 4174, 1170, 3850, 1315, 1611, 1859, 4183, 1403, 4033, 3623, 3596, 1461, 1848, 1173, 1928, 1765, 3719, 3584, 4165, 4191, 3980, 4058, 4200, 4336, 3958, 1260, 4051, 3798, 4032, 1774, 4322, 3991, 4106, 3978, 3965, 4464, 3813, 4154, 1365, 3846, 4345, 4379, 3603, 3937, 3801, 1787, 1214, 4483, 4201, 1845, 1220, 4245, 1347, 3867, 1900, 3640, 3916, 4089, 3745, 3975, 4256, 4308, 4000, 3868, 4403, 3883, 4326, 4393, 3895, 4237, 3955, 4005, 4092, 4372, 4283, 3805, 4151, 3982, 3597, 3872, 3987, 4490, 3876, 3668, 4300, 1679, 1242, 3598, 3583, 3647, 4304, 3779, 1907, 4042, 3648, 3996, 3800, 4367, 4099, 1807, 3887, 4428, 1625, 1390, 1884, 1682, 4364, 4306, 3869, 1278, 942, 1505, 1709, 3600, 3914, 3948, 1865, 1716, 3961, 1506, 4221, 1376, 1683, 4276, 1721, 1632, 3607, 1455, 1913, 1497, 1317, 1951, 1778, 3768, 4225, 1668, 390, 4406, 4341, 1480, 1714, 1422, 397, 3618, 4433, 1177, 3898, 398, 950, 400, 1965, 4471, 953, 401, 403, 955, 1482, 3602, 4434, 3721, 1592, 1825, 1698, 1519, 1352, 4409, 1977, 405, 1631, 1684, 409, 3889, 2117, 411, 413, 414, 415, 961, 1905, 416, 963, 3675, 1833, 3974, 965, 970, 1897, 1272, 4133, 3830, 972, 424, 427, 1708, 4141, 3749, 1619, 975, 4044, 3590, 1944, 4126, 1923, 1706, 432, 976, 978, 4400, 1606, 980, 2604, 2895, 436, 3448, 2188, 438, 1692, 442, 3862, 4055, 4358, 2276, 985, 1664, 443, 4309, 4045, 3854, 3682, 1972, 2688, 4366, 3425, 3231, 3649, 2759, 2724, 3755, 1216, 3599, 3775, 2754, 2606, 3471, 3234, 2565, 2803, 2562, 2684, 3411, 3405, 2690, 1255, 3694, 3985, 3535, 1306, 4084, 3282, 1780, 448, 990, 991, 1485, 992, 1728, 993, 994, 1696, 997, 3116, 1869, 456, 1386, 1000, 3879, 2698, 460, 1001, 461, 1447, 1002, 1003, 463, 464, 4425, 465, 1004, 466, 469, 1231, 1468, 470, 1005, 476, 1273, 1744, 1488, 1659, 1604, 479, 1013, 1014, 481, 4313, 1499, 1016, 4073, 1777, 485, 4274, 487, 1775, 4205, 1526, 1697, 2000, 488, 1894, 1882, 1817, 1880, 490, 1018, 3917, 3616, 4311, 4473, 4085, 4401, 3783, 3765, 1538, 3870, 491, 3699, 3792, 4290, 494, 2421, 4090, 2012, 1803, 3642, 1322, 1020, 502, 1333, 504, 1890, 507, 3608, 1732, 1864, 1816, 1531, 513, 3636, 514, 1699, 1027, 1366, 4950, 4027, 3478, 2959, 1293, 3197, 1595, 3413, 518, 1032, 1033, 1675, 1797, 2315, 521, 1999, 3759, 1215, 4122, 1730, 3966, 1035, 4973, 528, 3911, 3289, 1821, 530, 532, 1874, 533, 1038, 538, 539, 3767, 1929, 3683, 1338, 1616, 1901, 1796, 1517, 4331, 4332, 4466, 2719, 4123, 4233, 1824, 3129, 3087, 3505, 4158, 4254, 3720, 4351, 4414, 3453, 3254, 1649, 544, 4240, 3580, 1046, 548, 549, 2598, 552, 553, 3819, 1259, 4062, 554, 2841, 3515, 1433, 1323, 4301, 555, 558, 3738, 1567, 559, 1054, 1270, 3857, 2175, 1257, 1238, 3662, 561, 1058, 563, 1061, 4203, 1899, 3981, 1062, 1303, 4116, 1946, 3620, 1514, 4002, 3679, 3964, 3707, 3666, 1348, 4273, 1804, 4497, 1401, 4438, 1895, 1264, 1912, 573, 3939, 4170, 4056, 1271, 1521, 1221, 1451, 3934, 4080, 4352, 1700, 3739, 1419, 1811, 3950, 1690, 3716, 1569, 1437, 1674, 4337, 1285, 4268, 1183, 3881, 3938, 4198, 4416, 4229, 4292, 4193, 4325, 4375, 4432, 4215, 3944, 4195, 4223, 1540, 4455, 4512, 1723, 3750, 4070, 4104, 4235, 1863, 4346, 1881, 1814, 4443, 3761, 1587, 3592, 576, 4469, 1275, 1950, 4129, 1296, 1551, 3733, 4111, 3993, 4108, 1287, 1713, 1556, 1736, 1995, 1705, 1861, 4120, 4417, 1785, 1236, 4293, 1827, 3839, 3983, 4395, 1957, 3928, 4295, 3715, 3855, 3609, 4076, 4175, 3927, 1757, 4299, 1773, 1364, 1462, 4007, 3729, 3665, 3722, 1691, 1712, 1624, 1751, 1394, 3924, 3952, 581, 2003, 1559, 1250, 1555, 4114, 1818, 1076, 4448, 1078, 1786, 3354, 1832, 3432, 2891, 1903, 4459, 589, 1563, 1084, 591, 592, 594, 595, 1858, 596, 1453, 2559, 2590, 2738, 4478, 4069, 1809, 1327, 4150, 3260, 3481, 3672, 3625, 4439, 3852, 3997, 3847, 4050, 3377, 4424, 4143, 4493, 3492, 1412, 4017, 4477, 1794, 602, 4263, 3072, 2009, 1754, 3888, 603, 604, 1368, 3827, 4103, 1571, 1438, 611, 1093, 612, 1094, 3701, 4389, 614, 615, 1332, 1715, 4369, 1652, 1541, 1921, 1733, 1373, 1870, 619, 4218, 1798, 1702, 1384, 3700, 1788, 1600, 624, 2473, 1423, 2771, 1339, 3875, 3070, 2015, 2557, 2795, 2906, 2103, 3299, 3118, 4576, 1103, 1614, 1947, 631, 4479, 3766, 1107, 635, 1813, 636, 2431, 637, 1109, 639, 3706, 1110, 640, 642, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 1269, 3095, 1286, 3624, 4481, 3511, 2735, 4381, 3295, 1568, 1766, 3221, 2643, 2739, 3920, 647, 1801, 4279, 3762, 1637, 1509, 648, 1117, 1119, 2965, 2695, 3293, 4722, 1121, 653, 1122, 3264, 655, 656, 657, 4035, 2910, 3391, 2808, 3099, 2913, 3676, 2852, 3284, 2731, 3537, 3994, 1319, 4467, 1623, 660, 1123, 664, 1126, 1128, 1129, 1130, 670, 1527, 671, 1772, 1137, 5005, 1933, 1731, 1793, 3342, 1140, 1141, 687, 3822, 1601, 1148, 693, 3905, 694, 1414, 1149, 697, 1795, 1372, 1695, 1866, 1151, 4115, 1719, 1920, 4999, 1544, 703, 4031, 4305, 1636, 3810, 4452, 3677, 4227, 4402, 4157, 4197, 1337, 1253, 4028, 3989, 704, 1493, 1902, 1152, 4182, 707, 708, 709, 4476, 1375, 713, 714, 1841, 4234, 1154, 1314, 4096, 718, 719, 720, 2063, 1311, 721, 4217, 1627, 1158, 1953, 1201, 3787, 729, 3747, 730, 1846, 3815, 1964, 1822, 1262, 4480, 1537, 1291, 1318, 4294, 1582, 3439, 2833, 4287, 4509, 4505, 4267, 3697, 3272, 4112, 2967, 3651, 1805, 3705, 1195, 3859, 1164, 1165, 2351, 2423, 1745, 1973, 3807, 4307, 1169, 1454, or 1781.

18. The VP capsid polypeptide of claim 1, wherein X⁷ of SEQ ID NO: 5014 is R.

19. The VP capsid polypeptide of claim 18, wherein the 581 to 589 region of the VP capsid polypeptide has a sequence of any one of SEQ ID NOs: 740, 15, 742, 743, 17, 22, 24, 1836, 32, 36, 2450, 39, 752, 753, 1998, 3059, 2519, 757, 48, 1952, 50, 52, 760, 2389, 54, 2346, 58, 4086, 1660, 1966, 768, 2402, 772, 71, 72, 2453, 3882, 775, 1806, 3669, 1356, 2132, 81, 84, 85, 87, 4040, 88, 1885, 782, 783, 784, 785, 94, 788, 2173, 98, 2007, 102, 113, 799, 4848, 1444, 4821, 2329, 115, 119, 2290, 123, 806, 128, 134, 812, 145, 817, 154, 2149, 821, 167, 171, 4596, 183, 830, 191, 194, 2071, 195, 4920, 831, 2223, 207, 3901, 211, 212, 213, 215, 224, 4782, 845, 846, 849, 850, 854, 247, 248, 2245, 255, 261, 264, 265, 1477, 270, 874, 876, 279, 882, 1930, 285, 286, 886, 2119, 2468, 887, 888, 4138, 297, 3844, 302, 893, 307, 308, 2155, 1431, 2312, 900, 1635, 906, 2799, 2128, 2918, 1363, 4251, 2622, 3333, 3474, 3614, 2732, 3166, 3151, 3088, 2883, 3318, 2645, 2733, 3213, 3782, 1844, 2523, 2520, 2997, 2681, 3035, 3279, 2764, 3480, 2787, 1808, 3101, 3689, 4147, 1762, 1976, 340, 1594, 2439, 348, 4498, 1518, 1782, 926, 357, 359, 932, 363, 369, 1873, 3780, 4140, 4001, 3709, 3834, 1752, 4113, 3817, 4232, 1340, 1489, 4214, 3902, 3820, 4330, 1872, 3687, 4318, 375, 4210, 3860, 4212, 3695, 1851, 4504, 1910, 1428, 4323, 4422, 4259, 1176, 3711, 4365, 4383, 3969, 4128, 3802, 3863, 3821, 4171, 3824, 4462, 3760, 3753, 2783, 3880, 3943, 4163, 4178, 4199, 3724, 3670, 4482, 3692, 4430, 4316, 3645, 3690, 3589, 3730, 4426, 3851, 4429, 1335, 1717, 1997, 1975, 4280, 4006, 3900, 4298, 4388, 1341, 1350, 1909, 4412, 4407, 4281, 4167, 1893, 3585, 4015, 1747, 4286, 3826, 1473, 1914, 3984, 4495, 4184, 3977, 1707, 4074, 4003, 3915, 1410, 1498, 940, 4131, 4357, 1925, 1466, 1611, 1530, 4168, 4245, 4075, 3987, 1679, 4306, 3869, 3768, 393, 2265, 3618, 4433, 947, 399, 401, 402, 4139, 1352, 406, 415, 418, 420, 4919, 431, 4044, 976, 433, 434, 435, 442, 3873, 3411, 3985, 987, 1485, 2262, 457, 458, 4425, 466, 467, 468, 1468, 474, 1008, 1012, 1016, 4518, 1697, 490, 3616, 2247, 2302, 499, 500, 501, 509, 2110, 512, 1797, 520, 525, 3966, 1038, 537, 1041, 3683, 2165, 542, 4331, 3129, 1044, 4351, 3453, 1047, 1323, 4301, 557, 3738, 559, 1270, 3857, 1056, 2186, 1057, 1059, 565, 566, 3981, 567, 3620, 4002, 3666, 3939, 1221, 1397, 1437, 4337, 4268, 3938, 4292, 4215, 3750, 3633, 4070, 3592, 1827, 3839, 4395, 3855, 3665, 1624, 2395, 1818, 586, 587, 1903, 1080, 1081, 1082, 589, 1084, 1085, 594, 596, 4439, 3997, 1962, 1087, 4777, 606, 607, 610, 1094, 4389, 1603, 1095, 616, 618, 622, 1104, 4536, 3706, 2481, 3100, 3200, 3621, 4179, 1432, 4381, 643, 1115, 644, 2307, 2477, 652, 657, 658, 3994, 4467, 661, 663, 665, 4600, 1137, 1933, 4840, 1140, 678, 680, 683, 686, 692, 693, 696, 2081, 2280, 698, 1400, 4197, 1532, 1854, 706, 711, 712, 4570, 714, 1841, 1314, 4096, 2249, 726, 1846, 2385, 1318, 733, 3651, 1165, 4839, 4968, 4684, or 1454.

20. The VP capsid polypeptide of claim 1, wherein:
(a) X³ of SEQ ID NO: 5014 is a basic amino acid residue,
(b) X⁶ of SEQ ID NO: 5014 is a basic amino acid residue, or
(c) X³ of SEQ ID NO: 5014 is a basic amino acid residue and X⁶ of SEQ ID NO: 5014 is a basic amino acid residue.

21. The VP capsid polypeptide of claim 20, wherein the 581 to 589 region of the VP capsid polypeptide has a sequence of any one of SEQ ID NOs: 739, 4177, 1289, 740, 741, 18, 745, 3477, 1548, 3051, 2982, 2877, 3036, 3426, 3073, 3169, 747, 3362, 2784, 21, 3192, 23, 1638, 748, 1703, 1836, 3660, 29, 750, 1640, 33, 34, 35, 36, 37, 38, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 3327, 3178, 3006, 3252, 2991, 3470, 2888, 2572, 3418, 2642, 2519, 3363, 40, 2633, 41, 42, 43, 755, 45, 756, 46, 48, 1952, 758, 49, 3122, 2551, 3718, 3484, 3577, 759, 1230, 3960, 1378, 2994, 3080, 2589, 3681, 52, 760, 53, 762, 4659, 54, 3925, 55, 3654, 56, 60, 61, 4086, 1740, 1660, 765, 766, 62, 1966, 63, 64, 768, 771, 65, 66, 67, 68, 3816, 2599, 2706, 2399, 74, 3865, 1549, 1361, 2453, 3882, 3713, 1942, 75, 1312, 4501, 76, 77, 78, 1560, 4289, 3757, 79, 3593, 4043, 1806, 3908, 4397, 1615, 4415, 3723, 2006, 3754, 1470, 4360, 3945, 2854, 3523, 3669, 82, 4065, 83, 1294, 1484, 3891, 4456, 4040, 4496, 1982, 2001, 1478, 1189, 88, 1490, 1885, 1926, 89, 90, 91, 779, 1180, 92, 785, 786, 94, 95, 1831, 1867, 4458, 97, 1970, 1936, 1590, 99, 100, 789, 790, 1298, 101, 1688, 791, 792, 2007, 103, 794, 795, 104, 105, 106, 1849, 108, 4736, 1579, 110, 1906, 113, 1444, 1344, 116, 802, 117, 118, 2158, 121, 803, 804, 1463, 805, 122, 2058, 124, 125, 127, 4994, 807, 132, 809, 810, 133, 1978, 812, 135, 136, 137, 138, 813, 4673, 140, 815, 1326, 141, 142, 143, 144, 145, 4816, 1634, 1302, 817, 146, 818, 147, 148, 149, 151, 152, 153, 154, 155, 819, 820, 157, 821, 159, 160, 163, 164, 165, 166, 822, 167, 168, 169, 170, 171, 823, 824, 1651, 173, 1483, 174, 175, 825, 176, 177, 180, 826, 827, 181, 828, 182, 183, 4641, 829, 184, 185, 186, 187, 188, 189, 830, 191, 192, 193, 194, 195, 196, 1391, 197, 2223, 200, 201, 202, 203, 204, 205, 832, 833, 206, 834, 835, 836, 1334, 837, 3901, 3312, 2649, 1301, 211, 212, 1450, 4386, 3028, 213, 1446, 214, 215, 1896, 1522, 3490, 838, 2241, 217, 218, 219, 1612, 220, 221, 222, 839, 223, 840, 224, 226, 227, 230, 231, 842, 232, 233, 235, 844, 236, 237, 2150, 851, 852, 853, 1536, 240, 854, 242, 243, 244, 245, 246, 247, 855, 4634, 249, 250, 251, 252, 253, 254, 255, 857, 256, 257, 258, 259, 858, 859, 260, 261, 860, 861, 862, 262, 2326, 264, 1241, 265, 266, 866, 1477, 267, 867, 869, 870, 271, 871, 872, 272, 874, 1252, 273, 1591, 275, 1980, 276, 876, 877, 277, 278, 279, 1892, 879, 1539, 880, 1295, 3601, 3877, 1930, 1420, 1678, 1460, 2306, 282, 1799, 283, 1626, 1374, 287, 1620, 1290, 1354, 888, 291, 1956, 889, 292, 4324, 293, 4138, 891, 2224, 3903, 297, 3829, 3809, 4271, 3844, 1436, 1515, 1617, 1961, 304, 306, 1589, 4370, 308, 309, 894, 3646, 4059, 1431, 1331, 4948, 312, 895, 1779, 313, 314, 315, 316, 896, 1558, 1552, 897, 898, 321, 322, 899, 1958, 2134, 324, 901, 325, 902, 326, 1491, 329, 330, 905, 4484, 1756, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 1888, 4248, 1557, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 3435, 3482, 3020, 2748, 2646, 2787, 2760, 2563, 3085, 3133, 3158, 3170, 2703, 3494, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 2979, 2542, 2492, 1762, 4472, 1472, 1529, 4057, 1976, 4507, 1583, 338, 911, 1711, 340, 912, 2013, 341, 1594, 4440, 917, 344, 918, 919, 921, 922, 347, 349, 351, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 4145, 354, 925, 1782, 926, 927, 3610, 357, 358, 359, 4224, 1918, 1959, 3736, 1770, 931, 4155, 364, 1279, 365, 1543, 2472, 367, 4049, 1633, 1722, 368, 369, 1495, 1566, 370, 1211, 1873, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 3741, 1938, 3709, 1727, 3893, 1280, 3622, 1784, 3834, 1752, 1983, 1743, 3979, 4461, 4113, 3817, 4232, 1340, 1489, 4214, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 377, 1395, 4093, 4453, 3940, 3778, 4374, 4210, 4009, 4264, 4339, 4368, 1738, 378, 1610, 4194, 4474, 4013, 4266, 1771, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 2002, 1657, 1663, 1336, 3894, 1554, 1494, 1405, 3576, 4314, 3695, 1851, 4362, 1815, 3845, 4511, 3656, 3638, 1406, 1596, 4454, 1443, 1661, 1359, 1734, 3650, 1910, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1618, 1871, 1256, 3748, 4119, 1297, 1586, 1251, 1299, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1680, 4441, 3628, 3725, 1945, 937, 4206, 1588, 1439, 3794, 4447, 1838, 3776, 3658, 4064, 1377, 4011, 4272, 4503, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 3613, 3273, 3452, 3525, 1229, 4105, 3835, 383, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 4125, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 1520, 1767, 1466, 1194, 4137, 1908, 1927, 1758, 3772, 3661, 4186, 4174, 1170, 3850, 1315, 1611, 1530, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 1716, 3961, 1506, 4221, 1376, 1683, 4276, 1445, 1721, 1632, 3607, 1328, 1455, 1913, 1497, 1317, 1951, 1778, 1379, 3768, 4225, 1668, 390, 391, 4406, 4341, 1480, 395, 1714, 1422, 396, 2265, 397, 3618, 4433, 1177, 3898, 398, 949, 950, 399, 951, 400, 1965, 2345, 4471, 952, 402, 2475, 403, 404, 4139, 1482, 3602, 4434, 3721, 1592, 1519, 1352, 4202, 4409, 1977, 1917, 407, 408, 1631, 1684, 409, 3889, 958, 959, 412, 960, 414, 415, 961, 1905, 416, 963, 417, 3675, 1833, 3974, 420, 421, 965, 966, 969, 970, 422, 1897, 4754, 423, 4133, 3830, 972, 424, 973, 427, 1708, 4141, 3749, 431, 1619, 975, 4044, 3590, 1944, 4126, 1923, 1706, 4400, 434, 1606, 979, 2604, 2895, 436, 3448, 1692, 983, 3862, 4055, 4358, 984, 985, 1664, 4045, 3854, 3682, 2491, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 3535, 1306, 4084, 3282, 1780, 987, 988, 450, 990, 451, 452, 991, 992, 1728, 994, 995, 454, 1696, 998, 3116, 1869, 456, 1386, 999, 3879, 2698, 1001, 461, 1447, 462, 464, 4425, 1645, 1231, 1468, 472, 473, 4725, 476, 1273, 1007, 1744, 1488, 1659, 477, 1604, 481, 4313, 482, 483, 1499, 484, 2513, 1016, 4073, 1777, 1458, 4274, 4539, 4528, 487, 1775, 4205, 1526, 1697, 1017, 2000, 489, 1882, 1817, 1880, 490, 1018, 3917, 3616, 4311, 1800, 4473, 4085, 4401, 1310, 3783, 3765, 491, 1948, 3699, 1735, 3792, 4290, 495, 4090, 2247, 496, 1019, 2012, 1803, 3642, 1322, 498, 1021, 501, 1022, 502, 1333, 503, 1023, 1890, 1025, 510, 1026, 3608, 1732, 511, 2397, 1864, 1816, 1531, 3636, 1699, 1027, 1029, 1366, 2112, 2124, 2376, 1261, 4027, 3478, 3763, 2959, 517, 3197, 1595, 3413, 1448, 518, 2057, 1675, 1797, 2148, 2420, 520, 2315, 523, 1999, 3759, 1215, 4122, 1730, 524, 526, 3966, 3911, 529, 3289, 1821, 530, 531, 1874, 1038, 536, 537, 3767, 1929, 1041, 1042, 3683, 542, 1338, 1517, 4331, 4332, 4466, 2719, 3803, 4123, 4233, 1824, 3129, 3087, 3505, 4158, 4254, 1449, 3720, 4351, 4414, 1673, 3453, 3254, 1649, 544, 545, 546, 4240, 3580, 1046, 1047, 550, 2598, 551, 552, 3819, 1259, 4062, 2841, 1050, 3515, 4301, 557, 1052, 1053, 3738, 1567, 1270, 3857, 1055, 2198, 1257, 1238, 3662, 561, 562, 1058, 566, 1060, 1061, 2231, 4203, 1899, 3981, 1062, 1303, 4116, 567, 1946, 3620, 1514, 4002, 3679, 3964, 3707, 3666, 1348, 4273, 1804, 4497, 1401, 4438, 1895, 1264, 572, 1912, 3939, 4170, 4056, 1271, 1065, 1521, 1221, 1451, 1654, 3934, 4080, 4352, 1700, 3739, 1565, 1419, 1811, 3950, 1690, 1397, 3716, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 1723, 1070, 3750, 3633, 4070, 4104, 4162, 4235, 1863, 4346, 1881, 1814, 1572, 4443, 3761, 1346, 1587, 1072, 3592, 576, 1891, 4469, 1275, 1950, 1963, 4129, 1296, 1551, 3733, 4111, 3993, 4108, 3949, 1287, 1713, 1736, 1995, 1435, 1424, 1830, 1705, 1861, 1197, 1254, 4120, 1545, 4417, 1469, 1785, 1236, 4293, 3839, 3983, 4395, 1957, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1624, 1751, 1394, 3924, 3952, 580, 581, 2003, 1343, 1559, 1250, 1555, 4114, 1818, 1075, 1076, 1077, 1704, 4448, 1078, 1079, 1786, 585, 3354, 1832, 3432, 2891, 2505, 1903, 1081, 1082, 4459, 1083, 1563, 590, 591, 1085, 594, 597, 598, 1453, 2559, 2590, 2738, 4478, 4069, 1809, 1327, 4150, 3260, 3481, 3672, 3625, 2008, 4439, 3852, 3997, 1411, 599, 3847, 1268, 4050, 3377, 4424, 1496, 4143, 4493, 3492, 1962, 600, 601, 4017, 4477, 1794, 4263, 3072, 2009, 1754, 1087, 1088, 3888, 2324, 605, 1368, 3827, 4103, 606, 4549, 1090, 1571, 1091, 1438, 1092, 1093, 612, 1094, 3701, 4389, 613, 1603, 616, 1332, 1715, 4369, 1652, 1096, 617, 1541, 618, 1921, 1733, 1373, 1870, 619, 4218, 1798, 1099, 1100, 1702, 2239, 1101, 1667, 1384, 623, 3700, 1788, 1600, 624, 626, 2771, 627, 1339, 3875, 628, 629, 3070, 4818, 4860, 2557, 2795, 2906, 3118, 4657, 4646, 4875, 4627, 4747, 1614, 1947, 4618, 4479, 3766, 632, 1104, 1105, 633, 635, 1813, 636, 2213, 3706, 1110, 640, 1111, 641, 642, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 3095, 1286, 3624, 4481, 1432, 3511, 2735, 4381, 3295, 1585, 1114, 1766, 3221, 2643, 2739, 3920, 644, 1116, 645, 646, 2307, 647, 1801, 4279, 3762, 1637, 1509, 1119, 2965, 2369, 2695, 3293, 650, 651, 1120, 652, 1121, 4909, 1122, 3264, 655, 658, 659, 4035, 2910, 3391, 2808, 3099, 2913, 3676, 2852, 3284, 2731, 1609, 3994, 1319, 4467, 1802, 1227, 1623, 661, 2364, 662, 663, 664, 1124, 1125, 1127, 1128, 1129, 668, 669, 1130, 670, 1131, 1527, 1132, 671, 1772, 1135, 672, 5005, 2208, 4952, 4945, 1933, 1731, 1793, 675, 676, 3342, 1138, 1139, 677, 679, 1142, 1143, 681, 1145, 682, 685, 688, 3822, 689, 690, 691, 1147, 1601, 3905, 694, 695, 1414, 4793, 4575, 1150, 1795, 1372, 1695, 1866, 4115, 1719, 698, 2217, 1920, 699, 700, 1544, 702, 703, 4031, 4305, 1636, 1393, 2010, 3810, 4452, 3677, 4227, 1277, 1400, 4402, 4157, 4197, 1337, 1253, 1532, 4028, 3989, 1493, 1854, 1902, 1510, 2340, 4182, 706, 707, 708, 4476, 1245, 714, 1841, 4234, 1314, 4096, 717, 1155, 2249, 4217, 1156, 723, 1157, 1627, 725, 1159, 1953, 1160, 1201, 727, 3787, 4568, 3747, 730, 1846, 3815, 2005, 1964, 1822, 1262, 4480, 1537, 1291, 1318, 4294, 1582, 734, 3439, 2833, 4287, 4509, 4505, 4267, 3697, 3272, 4112, 2967, 3651, 1628, 3705, 1195, 3859, 1164, 2394, 1165, 1166, 1745, 1973, 1168, 3807, 4307, 2303, 1454, or 1781.

22. The VP capsid polypeptide of claim 20, wherein the 581 to 589 region of the VP capsid polypeptide has a sequence of any one of SEQ ID NOs: 4884, 738, 4177, 1289, 740, 14, 743, 744, 17, 3477, 1548, 3051, 2982, 2877, 746, 19, 3036, 3426, 3073, 3169, 747, 3362, 2784, 4889, 21, 3192, 4942, 22, 1638, 748, 24, 749, 27, 1703, 1836, 28, 3660, 29, 750, 1640, 33, 35, 36, 2450, 1653, 1205, 3795, 753, 4288, 4219, 2770, 2962, 4241, 4107, 2718, 3317, 4391, 3263, 2553, 3947, 2710, 2608, 3467, 2796, 2961, 2529, 3320, 2674, 2635, 3446, 3053, 3250, 3688, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 1467, 3327, 3178, 3006, 3252, 2991, 3470, 2888, 2572, 3418, 2642, 2519, 3363, 40, 2633, 44, 45, 756, 47, 48, 1952, 3122, 2551, 3718, 3484, 3577, 759, 50, 1308, 3960, 1378, 2994, 3080, 2589, 3681, 51, 760, 2389, 762, 3925, 3654, 763, 4626, 59, 60, 4086, 1740, 1660, 62, 1966, 63, 2438, 768, 66, 67, 2197, 68, 3816, 2599, 2706, 72, 73, 74, 3865, 1549, 1361, 2453, 3882, 773, 3713, 1942, 1312, 4501, 76, 1413, 775, 1560, 4289, 3757, 3593, 3908, 4397, 4415, 3723, 3754, 4360, 3945, 2854, 3523, 3669, 1356, 2132, 4065, 1294, 1484, 85, 86, 3891, 4456, 4040, 4496, 1982, 2001, 1478, 1189, 1490, 1885, 1926, 89, 780, 1180, 92, 781, 782, 783, 2356, 786, 94, 95, 788, 1831, 1867, 96, 4458, 97, 1970, 4656, 1936, 1590, 98, 99, 789, 790, 1298, 101, 1688, 791, 792, 2007, 102, 103, 104, 105, 107, 108, 1579, 110, 1906, 111, 798, 112, 799, 800, 2461, 1444, 801, 114, 115, 1344, 116, 802, 117, 118, 119, 121, 803, 1463, 1760, 124, 806, 126, 128, 807, 129, 131, 808, 2281, 133, 1978, 811, 812, 135, 136, 137, 138, 814, 140, 1326, 141, 142, 143, 2344, 816, 1634, 1302, 817, 818, 147, 148, 149, 150, 151, 154, 155, 819, 820, 156, 157, 2295, 158, 161, 162, 165, 822, 168, 169, 170, 823, 172, 1651, 173, 1483, 174, 825, 176, 178, 180, 827, 181, 828, 4911, 184, 185, 186, 188, 189, 830, 192, 193, 194, 196, 1391, 197, 198, 199, 200, 201, 202, 203, 4775, 205, 832, 206, 834, 207, 208, 209, 210, 835, 836, 1334, 3901, 3312, 2649, 1450, 4386, 3028, 1446, 2206, 215, 1522, 3490, 217, 219, 1612, 220, 221, 222, 839, 223, 840, 224, 225, 4751, 228, 229, 230, 231, 2448, 842, 232, 233, 4782, 234, 235, 238, 847, 239, 850, 851, 853, 1536, 240, 241, 242, 243, 244, 245, 246, 247, 4923, 4696, 250, 251, 254, 255, 857, 256, 258, 259, 859, 260, 861, 862, 4966, 864, 263, 4970, 2318, 865, 2326, 264, 1241, 266, 1477, 867, 268, 868, 269, 270, 869, 870, 271, 871, 872, 272, 873, 4655, 4972, 1252, 1591, 875, 274, 275, 1980, 276, 876, 877, 2066, 277, 278, 280, 2323, 1892, 1539, 2214, 881, 882, 3601, 3877, 1930, 1460, 282, 1799, 284, 4553, 4772, 1626, 286, 1374, 1620, 1290, 1354, 887, 1956, 889, 4765, 292, 4324, 4138, 891, 296, 3903, 297, 3829, 3809, 4271, 3844, 1436, 300, 1617, 1961, 5010, 302, 304, 305, 2147, 1589, 4370, 307, 3646, 4059, 1431, 1331, 312, 1779, 313, 896, 1558, 1552, 318, 319, 322, 1958, 2454, 323, 324, 901, 1491, 328, 329, 330, 4820, 4484, 1756, 907, 4584, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3414, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 4248, 1557, 3804, 1602, 4188, 1330, 4349, 3337, 3533, 2629, 3038, 2539, 3567, 3291, 3348, 3115, 3359, 2993, 3079, 3506, 2527, 2963, 2938, 2516, 3113, 2639, 3143, 3044, 2747, 3154, 3280, 3039, 2743, 2902, 3056, 2554, 2522, 3103, 3210, 3434, 2656, 4251, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 2869, 2610, 3298, 2775, 3108, 2603, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3629, 3578, 3885, 2817, 3248, 2954, 3334, 3571, 3345, 3551, 3384, 3422, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3120, 3286, 2843, 3465, 3946, 4222, 2682, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 4269, 3487, 3489, 3019, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 3689, 2968, 1457, 3450, 2577, 2567, 2532, 3415, 2934, 2984, 2568, 4147, 3002, 2986, 910, 2979, 2542, 1762, 4472, 1529, 4057, 1976, 4507, 1583, 1711, 2013, 1594, 4440, 343, 344, 920, 921, 348, 4498, 3728, 3637, 3570, 3429, 4091, 2870, 2995, 4338, 3205, 2880, 3261, 3968, 4291, 4204, 4071, 4034, 1742, 2820, 3912, 3287, 1218, 1190, 4145, 354, 1782, 927, 3610, 356, 4224, 1918, 1959, 1840, 3736, 1770, 929, 930, 931, 4155, 360, 361, 362, 364, 1279, 365, 1543, 366, 367, 4049, 1633, 1722, 369, 1495, 1566, 373, 1211, 2494, 1726, 1442, 1748, 4142, 1434, 1358, 1191, 3732, 4110, 4039, 4411, 4506, 4156, 4036, 4030, 4016, 3899, 1247, 1855, 3919, 1244, 4140, 3930, 4117, 4189, 4001, 3643, 3619, 1240, 1575, 3871, 4135, 4408, 3604, 1274, 4149, 3741, 1938, 3709, 3622, 3834, 1983, 1743, 3979, 4461, 4113, 3817, 4232, 4214, 3902, 1219, 1486, 1512, 1516, 4047, 4082, 4094, 1574, 1248, 4078, 1872, 1426, 3785, 3687, 4318, 4243, 3866, 3704, 4172, 1513, 1856, 3324, 2769, 2678, 1561, 3296, 2348, 377, 1395, 1584, 4093, 4453, 3778, 4210, 4009, 4264, 4339, 1738, 4194, 4474, 4013, 4266, 1771, 1759, 1922, 3860, 3832, 1349, 1381, 3663, 1185, 3932, 3858, 1198, 4192, 3611, 1919, 3790, 3712, 4095, 1887, 4468, 1720, 4247, 934, 1790, 2002, 1657, 1336, 3894, 1554, 3576, 4314, 3695, 1850, 4362, 1815, 3656, 3638, 4454, 1443, 1661, 4504, 1359, 3650, 1305, 1648, 3831, 4435, 4451, 1993, 3849, 1718, 4068, 1835, 1425, 1428, 4380, 1658, 1452, 4024, 4072, 3791, 4491, 4323, 4014, 1304, 1511, 1981, 1676, 4422, 1283, 3861, 3644, 4259, 1749, 1418, 1176, 4216, 1924, 1421, 3992, 4423, 4253, 3748, 4119, 1297, 1299, 1954, 4450, 3990, 4029, 3711, 4344, 1578, 4384, 3892, 3674, 3595, 4405, 3973, 4109, 4159, 3744, 4387, 1960, 4275, 4492, 4365, 4118, 3655, 3886, 4278, 3751, 3678, 4353, 3175, 2970, 1228, 3534, 3437, 3406, 3641, 3639, 4470, 3941, 4348, 3587, 3818, 3814, 4350, 3821, 3929, 3824, 4465, 4494, 1398, 3828, 1284, 3773, 4209, 3014, 3884, 2783, 4038, 3545, 2916, 3112, 2834, 3880, 4399, 4048, 3752, 4207, 1235, 3943, 4355, 4087, 4100, 4163, 4486, 4178, 1643, 4152, 3634, 4445, 4303, 4284, 1213, 4382, 1399, 3714, 4487, 3615, 4018, 4420, 4121, 3724, 4282, 4310, 4488, 4127, 3670, 4052, 3627, 3777, 1371, 3685, 4482, 4258, 3812, 1581, 4124, 4508, 4500, 3652, 3972, 1309, 1528, 3717, 1217, 2740, 3125, 3546, 3842, 4404, 3690, 3962, 4329, 4410, 3703, 4136, 3589, 3907, 4153, 2707, 4180, 3923, 4444, 4361, 4394, 4302, 3758, 4510, 3742, 1710, 1335, 1666, 1407, 4187, 1193, 1188, 1196, 1724, 1717, 1200, 1997, 1701, 4280, 4006, 1984, 3833, 1593, 2561, 4442, 3900, 4298, 1761, 4388, 1597, 4317, 4249, 1341, 1203, 1680, 4441, 3628, 3725, 1945, 937, 4206, 1588, 1439, 3794, 4447, 3776, 3658, 4064, 4011, 4272, 4503, 1324, 1367, 3594, 3680, 1300, 1750, 3913, 3774, 1877, 1987, 3998, 1380, 4185, 3686, 3696, 4463, 3853, 4244, 4083, 1909, 3897, 1546, 3613, 3273, 3452, 3525, 1229, 3835, 1234, 3970, 3856, 3586, 1175, 4321, 2778, 4412, 4407, 1810, 3823, 1427, 1507, 1878, 4333, 3146, 4281, 4211, 1179, 3843, 1677, 1524, 1525, 1974, 1239, 4167, 3740, 1342, 1471, 4176, 1232, 1989, 4125, 1741, 1580, 4060, 1576, 1212, 3921, 1174, 4061, 4021, 3693, 3734, 1382, 3684, 3806, 3635, 3976, 3840, 4286, 3836, 4161, 3659, 3743, 3605, 4392, 4328, 1316, 4173, 4265, 1246, 1629, 1829, 3826, 1992, 1656, 1837, 1914, 1839, 3984, 3910, 4160, 4184, 4132, 4250, 1562, 1847, 1792, 3769, 1534, 4003, 1689, 1599, 1265, 1410, 1823, 1498, 1329, 386, 940, 1655, 3874, 4261, 3626, 3579, 1644, 3936, 4054, 4260, 4297, 4357, 4022, 4046, 1222, 1996, 3788, 3953, 4226, 4277, 4101, 3797, 4019, 3771, 3632, 1402, 4137, 3772, 3661, 4186, 4174, 1170, 3850, 1315, 1611, 1859, 4183, 1403, 4033, 3623, 3596, 1461, 1848, 1173, 1928, 1765, 3719, 3584, 3591, 4165, 4191, 3980, 4058, 4200, 4336, 3958, 1260, 4051, 3798, 4032, 1774, 4322, 3991, 4106, 3978, 3965, 4464, 3813, 4154, 1365, 3846, 4345, 4379, 3603, 1693, 3937, 3801, 1787, 1214, 4483, 4201, 1898, 1845, 1220, 4245, 1347, 3867, 1900, 3640, 3916, 4089, 3745, 3975, 4256, 4308, 4000, 3868, 4403, 3883, 4326, 4393, 3895, 4237, 3955, 4005, 4092, 4372, 4283, 3805, 4151, 3982, 3597, 3872, 3987, 4490, 3876, 3668, 4300, 1679, 1242, 3598, 3583, 3647, 4304, 3779, 1907, 4042, 3648, 3996, 3800, 4367, 4099, 1807, 3887, 4428, 1625, 1390, 1884, 1682, 4364, 4306, 3869, 1278, 942, 1505, 1542, 1709, 3600, 3914, 3948, 1865, 1716, 3961, 1506, 4221, 1376, 1683, 4276, 1721, 1632, 3607, 1455, 1913, 1497, 1317, 1951, 1778, 3768, 4225, 1668, 390, 4406, 4341, 1480, 392, 1714, 1422, 397, 3618, 4433, 1177, 3898, 398, 950, 400, 1965, 4471, 953, 401, 403, 955, 1482, 3602, 4434, 3721, 1592, 1825, 1698, 1519, 1352, 4409, 1977, 405, 1631, 1684, 409, 3889, 2117, 411, 413, 414, 415, 961, 1905, 416, 963, 3675, 1833, 3974, 965, 970, 1897, 1272, 971, 4133, 3830, 972, 424, 427, 1708, 4141, 3749, 1619, 975, 4044, 3590, 1944, 4126, 1923, 1706, 432, 976, 978, 4400, 433, 1606, 980, 2604, 2895, 436, 3448, 2188, 438, 1692, 982, 2243, 439, 442, 3862, 4055, 4358, 2276, 985, 1664, 443, 4309, 4045, 3854, 3682, 1972, 2688, 4366, 3425, 3231, 3649, 2759, 2724, 3755, 1216, 3599, 3775, 2754, 2606, 3471, 3234, 2565, 2803, 2562, 2684, 3411, 3405, 2690, 1255, 3694, 3985, 3535, 1306, 4084, 3282, 1353, 1780, 448, 2031, 990, 991, 1485, 992, 1728, 993, 4703, 994, 1696, 997, 998, 3116, 1869, 456, 1386, 4778, 2040, 1000, 4981, 3879, 2698, 460, 1001, 461, 1447, 1002, 1003, 463, 464, 4425, 465, 1004, 466, 469, 1231, 1468, 470, 2496, 1005, 476, 1273, 1744, 1488, 1659, 4598, 1604, 479, 1013, 1014, 481, 4313, 1499, 1016, 4073, 1777, 2172, 485, 4274, 4599, 4796, 487, 4710, 1775, 4205, 1526, 1697, 2000, 488, 1894, 1882, 1817, 1880, 490, 1018, 3917, 3616, 4311, 1800, 4473, 4085, 4401, 3783, 3765, 1538, 3870, 491, 3699, 3792, 4290, 2074, 492, 494, 2421, 4090, 2012, 1803, 3642, 1322, 1020, 502, 1333, 504, 4805, 1890, 507, 3608, 1732, 511, 2397, 1864, 1816, 1531, 513, 3636, 514, 1699, 1027, 4987, 1366, 4664, 4950, 4027, 3478, 2959, 1293, 3197, 1595, 3413, 518, 4698, 1032, 1033, 1675, 1797, 4578, 4854, 2315, 521, 1999, 3759, 1215, 4122, 1730, 3966, 1035, 4973, 528, 3911, 3289, 1821, 530, 2426, 532, 1874, 533, 1038, 538, 539, 3767, 1929, 3683, 2100, 4853, 1338, 2021, 1616, 1901, 1796, 1517, 4331, 4332, 4466, 2719, 4123, 4233, 1824, 3129, 3087, 3505, 4158, 4254, 3720, 4351, 4414, 3453, 3254, 1649, 544, 4240, 3580, 1046, 548, 549, 2598, 552, 553, 3819, 1259, 4062, 554, 2841, 3515, 1433, 1323, 4301, 555, 558, 3738, 1567, 559, 1054, 1270, 3857, 2175, 1257, 1238, 3662, 561, 1058, 563, 4872, 2097, 1061, 4203, 1899, 3981, 1062, 1303, 4116, 570, 1946, 3620, 1514, 4002, 3679, 3964, 3707, 3666, 1348, 4273, 1804, 4497, 571, 1401, 4438, 1895, 1264, 1912, 573, 3939, 4170, 4056, 1271, 1521, 1221, 1451, 3934, 4080, 4352, 1700, 3739, 1419, 1811, 3950, 1690, 3716, 1569, 1437, 1674, 4337, 1285, 4268, 1183, 3881, 3938, 4198, 4416, 4229, 4292, 4193, 4325, 4375, 4432, 4215, 3944, 1409, 4195, 4223, 1540, 4455, 4512, 1723, 3750, 4070, 4104, 4235, 1863, 4346, 1881, 1071, 1814, 4443, 3761, 1346, 1587, 3592, 576, 4469, 1275, 1950, 4129, 1296, 1551, 3733, 4111, 3993, 4108, 3949, 1287, 1713, 1556, 1736, 1995, 1705, 1861, 4120, 4417, 1292, 1785, 1236, 4293, 1827, 3839, 3983, 4395, 1957, 3928, 4295, 3715, 3855, 3609, 4076, 4175, 3927, 1757, 4299, 1773, 1364, 1462, 4007, 3729, 3665, 3722, 1691, 1712, 1624, 1751, 1394, 3924, 3952, 581, 2003, 1559, 1250, 1555, 4114, 1818, 1076, 4448, 1078, 1786, 3354, 1832, 3432, 2891, 4583, 1903, 1080, 4459, 589, 1563, 1084, 591, 592, 594, 595, 1858, 596, 598, 1453, 2559, 2590, 2738, 4478, 4069, 1809, 1327, 4150, 3260, 3481, 3672, 3625, 4439, 3852, 3997, 3847, 4050, 3377, 4424, 4143, 4493, 3492, 1412, 601, 4017, 4477, 1794, 602, 4263, 3072, 2009, 1754, 1088, 3888, 603, 604, 1368, 3827, 4103, 1571, 1438, 2069, 611, 1093, 612, 1094, 3701, 4389, 614, 5000, 615, 1332, 1715, 4369, 1652, 1541, 1921, 1733, 1373, 1870, 619, 4218, 1798, 1702, 1384, 3700, 1788, 1600, 624, 2473, 1423, 2771, 1339, 3875, 3070, 2015, 2557, 2795, 2906, 2103, 3299, 3118, 4838, 4965, 4605, 4576, 1103, 1614, 1947, 631, 4479, 3766, 1107, 635, 1813, 636, 2431, 637, 1109, 4577, 639, 3706, 1110, 640, 1111, 4581, 4644, 4734, 642, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 1269, 3095, 1286, 3624, 4481, 3511, 2735, 4381, 3295, 1585, 1568, 1766, 3221, 2643, 2739, 4629, 3920, 4603, 2307, 647, 1801, 4279, 3762, 1637, 1509, 4590, 648, 1117, 4800, 4724, 4674, 1119, 2965, 2695, 3293, 4722, 1121, 4784, 653, 1122, 3264, 655, 656, 657, 4035, 2910, 3391, 2808, 3099, 2913, 3676, 2852, 3284, 2731, 3537, 3994, 1319, 4467, 1623, 660, 4714, 4812, 1123, 664, 2138, 1126, 1128, 1129, 1130, 670, 1527, 671, 1772, 1133, 2196, 1136, 1137, 5005, 1933, 1731, 1793, 3342, 1140, 1141, 4525, 687, 3822, 1601, 1148, 693, 3905, 694, 1414, 1149, 4756, 4859, 2258, 697, 1795, 1372, 1695, 1866, 1151, 4115, 1719, 2194, 1920, 4999, 1544, 703, 4031, 4305, 1636, 1393, 3810, 4452, 3677, 4227, 4402, 4157, 4197, 1337, 1253, 1532, 4028, 3989, 4715, 704, 1493, 1902, 1152, 4824, 4182, 707, 708, 709, 4732, 4476, 1375, 713, 714, 715, 1841, 4234, 1154, 1314, 4096, 4895, 718, 719, 720, 2063, 1311, 721, 4903, 4885, 4217, 1627, 2116, 1158, 724, 5013, 4894, 1953, 1201, 3787, 729, 2485, 3747, 730, 1846, 3815, 1964, 1822, 1262, 4480, 1537, 1291, 1318, 4976, 4294, 1582, 4587, 3439, 2833, 4287, 4509, 4505, 4267, 3697, 3272, 4112, 2967, 3651, 1805, 3705, 1195, 3859, 1164, 1165, 2351, 4835, 4571, 2423, 1745, 1973, 1168, 3807, 4307, 1169, 1454, or 1781.

23. The VP capsid polypeptide of claim 20, wherein the 581 to 589 region of the VP capsid polypeptide has a sequence of any one of SEQ ID NOs: 4177, 1289, 740, 3477, 1548, 3051, 2982, 2877, 3036, 3426, 3073, 3169, 747, 3362, 2784, 21, 3192, 1638, 748, 1703, 1836, 3660, 29, 750, 1640, 33, 35, 36, 1205, 3795, 753, 4288, 4219, 2770, 2962, 4241, 4107, 2718, 3317, 4391, 3263, 2553, 3947, 2710, 2608, 3467, 2796, 2961, 2529, 3320, 2674, 2635, 3446, 3053, 3250, 3688, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 3327, 3178, 3006, 3252, 2991, 3470, 2888, 2572, 3418, 2642, 2519, 3363, 40, 2633, 45, 756, 48, 1952, 3122, 2551, 3718, 3484, 3577, 759, 3960, 1378, 2994, 3080, 2589, 3681, 760, 762, 3925, 3654, 60, 4086, 1740, 1660, 62, 1966, 63, 768, 66, 67, 68, 3816, 2599, 2706, 74, 3865, 1549, 1361, 2453, 3882, 3713, 1942, 1312, 4501, 76, 1560, 4289, 3757, 3593, 3908, 4397, 4415, 3723, 3754, 4360, 3945, 2854, 3523, 3669, 4065, 1294, 1484, 3891, 4456, 4040, 4496, 1982, 2001, 1478, 1189, 1490, 1885, 1926, 89, 1180, 92, 786, 94, 95, 1831, 1867, 4458, 97, 1970, 1936, 1590, 99, 789, 790, 1298, 101, 1688, 791, 792, 2007, 103, 104, 105, 108, 1579, 110, 1906, 1444, 1344, 116, 802, 117, 118, 121, 803, 1463, 124, 807, 133, 1978, 812, 135, 136, 137, 138, 140, 1326, 141, 142, 143, 1634, 1302, 817, 818, 147, 148, 149, 151, 154, 155, 819, 820, 157, 165, 822, 168, 169, 170, 823, 1651, 173, 1483, 174, 825, 176, 180, 827, 181, 828, 184, 185, 186, 188, 189, 830, 192, 193, 194, 196, 1391, 197, 200, 201, 202, 203, 205, 832, 206, 834, 835, 836, 1334, 3901, 3312, 2649, 1450, 4386, 3028, 1446, 215, 1522, 3490, 217, 219, 1612, 220, 221, 222, 839, 223, 840, 224, 230, 231, 842, 232, 233, 235, 851, 853, 1536, 240, 242, 243, 244, 245, 246, 247, 250, 251, 254, 255, 857, 256, 258, 259, 859, 260, 861, 862, 2326, 264, 1241, 266, 1477, 867, 869, 870, 271, 871, 872, 272, 1252, 1591, 275, 1980, 276, 876, 877, 277, 278, 1892, 1539, 3601, 3877, 1930, 1460, 282, 1799, 1626, 1374, 1620, 1290, 1354, 1956, 889, 292, 4324, 4138, 891, 3903, 297, 3829, 3809, 4271, 3844, 1436, 1617, 1961, 304, 1589, 4370, 3646, 4059, 1431, 1331, 312, 1779, 313, 896, 1558, 1552, 322, 1958, 324, 901, 1491, 329, 330, 4484, 1756, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 4248, 1557, 1602, 4188, 1330, 4349, 3337, 3533, 2629, 3038, 2539, 3567, 3291, 3348, 3115, 3359, 2993, 3079, 3506, 2527, 2963, 2938, 2516, 3113, 2639, 3143, 3044, 2747, 3154, 3280, 3039, 2743, 2902, 3056, 2554, 2522, 3103, 3210, 3434, 2656, 4251, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 2869, 2610, 3298, 2775, 3108, 2603, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3629, 3578, 3885, 2817, 3248, 2954, 3334, 3571, 3345, 3551, 3384, 3422, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3120, 3286, 2843, 3465, 3946, 4222, 2682, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 4269, 3487, 3489, 3019, 1266, 3435, 3482, 3020, 2748, 2646, 2787, 2760, 2563, 3085, 3133, 3158, 3170, 2703, 3494, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 3689, 2968, 1457, 3450, 2577, 2567, 2532, 3415, 2934, 2984, 2568, 4147, 3002, 2986, 2979, 2542, 1762, 4472, 1529, 4057, 1976, 4507, 1583, 1711, 2013, 1594, 4440, 344, 921, 4498, 3728, 3637, 3570, 3429, 4091, 2870, 2995, 4338, 3205, 2880, 3261, 3968, 4291, 4204, 4071, 4034, 1742, 2820, 3912, 3287, 4145, 354, 1782, 927, 3610, 4224, 1918, 1959, 3736, 1770, 931, 4155, 364, 1279, 365, 1543, 367, 4049, 1633, 1722, 369, 1495, 1566, 1211, 1748, 4142, 1434, 1358, 1191, 3732, 4110, 4039, 4411, 4506, 4156, 4036, 4030, 4016, 3899, 1247, 1855, 3919, 1244, 4140, 3930, 4117, 4189, 4001, 3643, 3619, 1240, 1575, 3871, 4135, 4408, 3604, 1274, 3741, 1938, 3709, 3622, 3834, 1983, 1743, 3979, 4461, 4113, 3817, 4232, 4214, 1219, 1486, 1512, 1516, 4047, 4082, 4094, 1574, 1248, 4078, 1872, 1426, 3785, 3687, 4318, 4243, 3866, 3704, 4172, 1513, 1856, 3324, 2769, 2678, 1561, 3296, 377, 1395, 4093, 4453, 3778, 4210, 4009, 4264, 4339, 1738, 4194, 4474, 4013, 4266, 1771, 3860, 3832, 1349, 1381, 3663, 1185, 3932, 3858, 1198, 4192, 3611, 1919, 3790, 3712, 4095, 1887, 4468, 1720, 4247, 2002, 1657, 1336, 3894, 1554, 3576, 4314, 3695, 4362, 1815, 3656, 3638, 4454, 1443, 1661, 1359, 3650, 1648, 3831, 4435, 4451, 1993, 3849, 1718, 4068, 1835, 1425, 1428, 4380, 1658, 1452, 4024, 4072, 3791, 4491, 4323, 4014, 1304, 1511, 1981, 1676, 4422, 3748, 4119, 1297, 1299, 4450, 3990, 4029, 3711, 4344, 1578, 4384, 3892, 3674, 3595, 4405, 3973, 4109, 4159, 3744, 4387, 1960, 4275, 4492, 4365, 4118, 3655, 3886, 4278, 3751, 3678, 4353, 3175, 2970, 1228, 3534, 3437, 3406, 3641, 3639, 4470, 3941, 4348, 3587, 3818, 3814, 4350, 3821, 3929, 3824, 4465, 4494, 1398, 3828, 1284, 3773, 4209, 3014, 3884, 2783, 4038, 3545, 2916, 3112, 2834, 3880, 4399, 4048, 3752, 4207, 1235, 3943, 4355, 4087, 4100, 4163, 4486, 4178, 1643, 4152, 3634, 4445, 4303, 4284, 1213, 4382, 1399, 3714, 4487, 3615, 4018, 4420, 4121, 3724, 4282, 4310, 4488, 4127, 3670, 4052, 4124, 4508, 4500, 3652, 3972, 1309, 1528, 3717, 1217, 2740, 3125, 3546, 3842, 4404, 3690, 3962, 4329, 4410, 3703, 4136, 3589, 3907, 4153, 2707, 4180, 3923, 4444, 4361, 4394, 4302, 3758, 4510, 3742, 1710, 1335, 1997, 1701, 4280, 4006, 1984, 3833, 1593, 2561, 4442, 3900, 4298, 1761, 4388, 1597, 4317, 4249, 1341, 1680, 4441, 3628, 3725, 1945, 937, 4206, 1588, 1439, 3794, 4447, 3776, 3658, 4064, 4011, 4272, 4503, 1367, 3594, 3680, 1300, 1750, 3913, 3774, 1877, 1987, 3998, 1380, 4185, 3686, 3696, 4463, 3853, 4244, 4083, 1909, 3897, 1546, 3613, 3273, 3452, 3525, 1229, 3835, 1234, 3970, 3856, 3586, 1175, 4321, 2778, 4412, 4407, 1810, 3823, 1427, 1507, 1878, 4333, 3146, 4281, 4211, 1179, 3843, 1677, 1524, 1525, 1974, 1239, 4167, 3740, 1342, 4125, 1580, 4060, 1576, 1212, 3921, 1174, 4061, 4021, 3693, 3734, 1382, 3684, 3806, 3635, 3976, 3840, 4286, 3836, 4161, 3659, 3743, 3605, 4392, 4328, 1316, 4173, 4265, 1246, 1629, 1829, 1837, 1914, 1839, 3984, 3910, 4160, 4184, 4132, 4250, 1562, 1847, 1792, 3769, 1534, 4003, 1689, 1599, 1265, 1655, 3874, 4261, 3626, 3579, 1644, 3936, 4054, 4260, 4297, 4357, 4022, 4046, 1222, 1996, 3788, 3953, 4226, 4277, 4101, 3797, 4019, 3771, 3632, 1402, 4137, 3772, 3661, 4186, 4174, 1170, 3850, 1315, 1611, 4033, 3623, 3596, 1461, 1848, 1173, 1928, 1765, 3719, 3584, 3591, 4165, 4191, 3980, 4058, 4200, 4336, 3958, 1260, 4051, 3798, 4032, 1774, 4322, 3991, 4106, 3978, 3965, 4464, 3813, 4154, 1365, 3846, 4345, 4379, 3603, 1693, 3937, 3801, 1787, 1214, 4483, 4201, 1898, 1220, 4245, 1347, 3867, 1900, 3640, 3916, 4089, 3745, 3975, 4256, 4308, 4000, 3868, 4403, 3883, 4326, 4393, 3895, 4237, 3955, 4005, 4092, 4372, 4283, 3805, 4151, 3982, 3597, 3872, 3987, 4490, 3876, 3668, 4300, 1679, 1242, 3598, 3583, 3647, 4304, 3779, 1907, 4042, 3648, 3996, 3800, 4367, 4099, 1807, 3887, 4428, 1625, 1390, 1884, 1682, 4364, 4306, 3869, 1278, 942, 1505, 1542, 1709, 3600, 3914, 3948, 1865, 1716, 3961, 1506, 4221, 1376, 1683, 4276, 1721, 1632, 3607, 1455, 1913, 1497, 1317, 1951, 1778, 3768, 4225, 1668, 390, 4406, 4341, 1480, 1714, 1422, 397, 3618, 4433, 1177, 3898, 398, 950, 400, 1965, 4471, 4031, 482, 3602, 4434, 3721, 1592, 1519, 1352, 4409, 1977, 1631, 1684, 409, 3889, 414, 415, 961, 1905, 416, 963, 3675, 1833, 3974, 965, 970, 1897, 4133, 3830, 972, 424, 427, 1708, 4141, 3749, 1619, 975, 4044, 3590, 1944, 4126, 1923, 1706, 4400, 1606, 2604, 2895, 436, 3448, 1692, 3862, 4055, 4358, 985, 1664, 4045, 3854, 3682, 1972, 2688, 4366, 3425, 3231, 3649, 2759, 2724, 3755, 1216, 3599, 3775, 2754, 2606, 3471, 3234, 2565, 2803, 2562, 2684, 3411, 3405, 2690, 1255, 3694, 3535, 1306, 4084, 3282, 1780, 990, 991, 992, 1728, 994, 1696, 998, 3116, 1869, 456, 1386, 3879, 2698, 1001, 461, 1447, 464, 4425, 1231, 1468, 476, 1273, 1744, 1488, 1659, 1604, 481, 4313, 1499, 1016, 4073, 1777, 4274, 487, 1775, 4205, 1526, 1697, 2000, 1882, 1817, 1880, 490, 1018, 3917, 3616, 4311, 1800, 4473, 4085, 4401, 3783, 3765, 491, 3699, 3792, 4290, 4090, 2012, 1803, 3642, 1322, 502, 1333, 1890, 3608, 1732, 511, 2397, 1864, 1816, 1531, 3636, 1699, 1027, 1366, 4027, 3478, 2959, 3197, 1595, 3413, 518, 1675, 1797, 2315, 1999, 3759, 1215, 4122, 1730, 3966, 3911, 3289, 1821, 530, 1874, 1038, 3767, 1929, 3683, 1338, 1517, 4331, 4332, 4466, 2719, 4123, 4233, 1824, 3129, 3087, 3505, 4158, 4254, 3720, 4351, 4414, 3453, 3254, 1649, 544, 4240, 3580, 1046, 2598, 552, 3819, 1259, 4062, 2841, 3515, 4301, 3738, 1567, 1270, 3857, 1257, 1238, 3662, 561, 1058, 1061, 4203, 1899, 3981, 1062, 1303, 4116, 1946, 3620, 1514, 4002, 3679, 3964, 3707, 3666, 1348, 4273, 1804, 4497, 1401, 4438, 1895, 1264, 1912, 3939, 4170, 4056, 1271, 1521, 1221, 1451, 3934, 4080, 4352, 1700, 3739, 1419, 1811, 3950, 1690, 3716, 1437, 1674, 4337, 1285, 4268, 1183, 3881, 3938, 4198, 4416, 4229, 4292, 4193, 4325, 4375, 4432, 4215, 3944, 1409, 4195, 4223, 1540, 1723, 3750, 4070, 4104, 4235, 1863, 4346, 1881, 1814, 4443, 3761, 1346, 1587, 3592, 576, 4469, 1275, 1950, 4129, 1296, 1551, 3733, 4111, 3993, 4108, 3949, 1287, 1713, 1736, 1995, 1705, 1861, 4120, 4417, 1785, 1236, 4293, 3839, 3983, 4395, 1957, 3928, 4295, 3715, 3855, 3609, 4076, 4175, 3927, 1757, 4299, 1773, 1364, 1462, 4007, 3729, 3665, 3722, 1691, 1624, 1751, 1394, 3924, 3952, 581, 2003, 1559, 1250, 1555, 4114, 1818, 1076, 4448, 1078, 1786, 3354, 1832, 3432, 2891, 1903, 4459, 1563, 591, 594, 598, 1453, 2559, 2590, 2738, 4478, 4069, 1809, 1327, 4150, 3260, 3481, 3672, 3625, 4439, 3852, 3997, 3847, 4050, 3377, 4424, 4143, 4493, 3492, 601, 4017, 4477, 1794, 4263, 3072, 2009, 1754, 1088, 3888, 1368, 3827, 4103, 1571, 1438, 1093, 612, 1094, 3701, 4389, 1332, 1715, 4369, 1652, 1541, 1921, 1733, 1373, 1870, 619, 4218, 1798, 1702, 1384, 3700, 1788, 1600, 624, 2771, 1339, 3875, 3070, 2557, 2795, 2906, 3118, 1614, 1947, 4479, 3766, 635, 1813, 636, 3706, 1110, 640, 1111, 642, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 3095, 1286, 3624, 4481, 3511, 2735, 4381, 3295, 1585, 1766, 3221, 2643, 2739, 3920, 2307, 647, 1801, 4279, 3762, 1637, 1509, 1119, 2965, 2695, 3293, 1121, 1122, 3264, 655, 4035, 2910, 3391, 2808, 3099, 2913, 3676, 2852, 3284, 2731, 3994, 1319, 4467, 1623, 664, 1128, 1129, 1130, 670, 1527, 671, 1772, 5005, 1933, 1731, 1793, 3342, 3822, 1601, 3905, 694, 1414, 1795, 1372, 1695, 1866, 4115, 1719, 1920, 1544, 703, 4031, 4305, 1636, 1393, 3810, 4452, 3677, 4227, 4402, 4157, 4197, 1337, 1253, 1532, 4028, 3989, 1493, 1902, 4182, 707, 708, 4476, 714, 1841, 4234, 1314, 4096, 4217, 1627, 1953, 1201, 3787, 3747, 730, 1846, 3815, 1964, 1822, 1262, 4480, 1537, 1291, 1318, 4294, 1582, 3439, 2833, 4287, 4509, 4505, 4267, 3697, 3272, 4112, 2967, 3651, 3705, 1195, 3859, 1164, 1165, 1745, 1973, 1168, 3807, 4307, 1454, or 1781.

24. The VP capsid polypeptide of any one of claims 20-23, wherein the basic residue is selected from the group consisting of K, R, and H.

25. The VP capsid polypeptide of any one of claims 20-24, wherein X³ of SEQ ID NO: 5014 is K or R.

26. The VP capsid polypeptide of claim 25, wherein the 581 to 589 region of the VP capsid polypeptide has a sequence of any one of SEQ ID NOs: 4177, 1289, 740, 741, 18, 745, 3477, 1548, 3051, 2982, 2877, 3036, 3426, 3073, 3169, 747, 3362, 2784, 21, 3192, 23, 1638, 748, 1703, 1836, 3660, 29, 750, 1640, 33, 34, 35, 36, 37, 38, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 3327, 3178, 3006, 3252, 2991, 3470, 2888, 2572, 3418, 2642, 2519, 3363, 40, 2633, 41, 42, 43, 755, 45, 756, 46, 48, 1952, 758, 49, 3122, 2551, 3718, 3484, 3577, 759, 1230, 3960, 1378, 2994, 3080, 2589, 3681, 760, 53, 3925, 55, 3654, 56, 61, 4086, 1740, 1660, 765, 766, 62, 1966, 63, 64, 768, 771, 65, 66, 67, 68, 3816, 2599, 2706, 74, 3865, 1549, 1361, 3882, 3713, 1942, 1312, 4501, 76, 77, 1560, 4289, 3757, 79, 3593, 4043, 1806, 3908, 4397, 1615, 4415, 3723, 2006, 3754, 1470, 4360, 3945, 2854, 3523, 3669, 82, 4065, 83, 1294, 1484, 3891, 4456, 4040, 4496, 1982, 2001, 1478, 1189, 1490, 1885, 1926, 89, 90, 91, 779, 1180, 92, 785, 786, 94, 95, 1831, 1867, 4458, 97, 1970, 1936, 1590, 100, 789, 790, 1298, 101, 1688, 791, 792, 2007, 103, 794, 795, 104, 105, 106, 1849, 108, 1579, 110, 1906, 113, 1444, 1344, 116, 802, 117, 118, 121, 803, 804, 1463, 805, 122, 124, 125, 127, 807, 132, 809, 810, 133, 1978, 812, 135, 136, 137, 138, 813, 140, 815, 1326, 141, 142, 143, 144, 145, 1634, 1302, 817, 146, 818, 147, 148, 149, 151, 152, 153, 154, 155, 819, 820, 157, 159, 160, 163, 164, 165, 166, 822, 167, 168, 169, 170, 171, 823, 824, 1651, 173, 1483, 174, 175, 825, 176, 177, 826, 827, 181, 828, 182, 829, 184, 185, 186, 187, 188, 189, 830, 191, 192, 193, 194, 195, 196, 1391, 197, 200, 201, 202, 203, 204, 205, 832, 833, 206, 834, 836, 1334, 837, 3901, 3312, 2649, 1301, 211, 212, 1450, 4386, 3028, 213, 1446, 214, 215, 1896, 1522, 3490, 838, 217, 218, 219, 1612, 220, 221, 222, 839, 223, 840, 224, 226, 227, 230, 231, 842, 232, 233, 844, 236, 237, 851, 852, 853, 1536, 240, 854, 242, 243, 244, 245, 246, 247, 249, 250, 251, 252, 253, 254, 255, 857, 256, 257, 258, 259, 858, 859, 260, 261, 860, 861, 862, 262, 264, 1241, 265, 266, 866, 1477, 267, 867, 869, 870, 271, 871, 872, 272, 1252, 273, 1591, 275, 1980, 276, 876, 877, 277, 278, 279, 1892, 1539, 880, 1295, 3601, 3877, 1930, 1420, 1678, 1460, 282, 1799, 283, 1626, 1374, 287, 1620, 1290, 1354, 888, 291, 1956, 889, 292, 4324, 293, 4138, 891, 3903, 297, 3829, 3809, 4271, 3844, 1436, 1515, 1617, 1961, 304, 306, 1589, 4370, 308, 309, 894, 3646, 4059, 1431, 1331, 312, 895, 1779, 313, 314, 315, 316, 896, 1558, 1552, 898, 321, 322, 899, 1958, 901, 325, 902, 326, 1491, 329, 330, 905, 4484, 1756, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 1888, 4248, 1557, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 3435, 3482, 3020, 2748, 2646, 2787, 2760, 2563, 3085, 3133, 3158, 3170, 2703, 3494, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 2979, 2542, 1762, 4472, 1472, 1529, 4057, 1976, 4507, 1583, 338, 911, 1711, 340, 912, 2013, 341, 4440, 917, 344, 918, 919, 921, 922, 347, 351, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 4145, 354, 925, 1782, 926, 927, 3610, 357, 359, 4224, 1918, 1959, 3736, 1770, 931, 4155, 364, 1279, 365, 1543, 367, 4049, 1633, 1722, 368, 369, 1495, 1566, 370, 1211, 1873, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 3741, 1938, 3709, 1727, 3893, 1280, 3622, 1784, 3834, 1752, 1983, 1743, 3979, 4461, 4113, 3817, 4232, 1340, 1489, 4214, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 377, 1395, 4453, 3940, 3778, 4374, 4210, 4009, 4264, 4339, 4368, 1738, 378, 1610, 4194, 4474, 4013, 4266, 1771, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 2002, 1657, 1663, 1336, 3894, 1554, 1494, 1405, 3576, 4314, 3695, 1851, 4362, 1815, 3845, 4511, 3656, 3638, 1406, 1596, 4454, 1443, 1661, 1359, 1734, 3650, 1910, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1680, 4441, 3628, 3725, 1945, 4206, 1588, 1439, 3794, 4447, 1838, 3776, 3658, 4064, 1377, 4011, 4272, 4503, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 3613, 3273, 3452, 3525, 1229, 4105, 3835, 383, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 1520, 1767, 1466, 1194, 4137, 1908, 1927, 1758, 3772, 3661, 4186, 4174, 1170, 3850, 1315, 1611, 1530, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 1716, 3961, 1506, 4221, 1376, 1683, 4276, 1445, 1721, 1632, 3607, 1328, 1455, 1913, 1497, 1317, 1951, 1778, 1379, 3768, 4225, 1668, 390, 391, 4406, 4341, 1480, 395, 1714, 1422, 396, 397, 3618, 4433, 1177, 3898, 398, 949, 950, 399, 951, 400, 1965, 4471, 952, 402, 2475, 403, 404, 4139, 1482, 3602, 4434, 3721, 1592, 1519, 1352, 4202, 4409, 1977, 1917, 407, 408, 1631, 1684, 409, 3889, 958, 959, 412, 960, 414, 415, 961, 1905, 416, 963, 417, 3675, 1833, 3974, 420, 421, 965, 966, 969, 970, 422, 1897, 423, 4133, 3830, 972, 424, 973, 427, 1708, 4141, 3749, 431, 1619, 975, 4044, 3590, 1944, 4126, 1923, 1706, 4400, 434, 1606, 979, 2604, 2895, 436, 3448, 1692, 983, 3862, 4055, 4358, 984, 985, 1664, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 3535, 1306, 4084, 3282, 1780, 987, 988, 990, 451, 452, 991, 992, 1728, 994, 995, 454, 1696, 3116, 1869, 456, 1386, 999, 3879, 2698, 1001, 461, 1447, 462, 464, 4425, 1645, 1231, 1468, 472, 473, 476, 1273, 1007, 1744, 1488, 1659, 477, 1604, 481, 4313, 482, 483, 1499, 484, 1016, 4073, 1777, 1458, 4274, 487, 1775, 4205, 1526, 1697, 1017, 2000, 489, 3917, 3616, 4311, 1800, 4473, 4085, 4401, 1310, 3783, 3765, 491, 1948, 3699, 1735, 3792, 4290, 495, 4090, 496, 2012, 1803, 3642, 1322, 498, 1021, 1022, 502, 1333, 503, 1023, 1890, 510, 1026, 3608, 1732, 511, 2397, 1864, 1816, 1531, 3636, 1699, 1027, 1029, 1366, 2112, 1261, 4027, 3478, 3763, 2959, 517, 3197, 1595, 3413, 1448, 518, 1675, 1797, 520, 1999, 3759, 1215, 4122, 1730, 524, 526, 3966, 3911, 529, 3289, 1821, 530, 531, 1874, 1038, 536, 537, 3767, 1929, 1041, 1042, 3683, 542, 1338, 4331, 4332, 4466, 2719, 3803, 4123, 4233, 1824, 3129, 3087, 3505, 4158, 4254, 1449, 3720, 4351, 4414, 1673, 3453, 3254, 1649, 546, 4240, 3580, 1046, 1047, 550, 2598, 551, 552, 3819, 1259, 4062, 2841, 1050, 3515, 4301, 1052, 1053, 3738, 1567, 1270, 3857, 1055, 1257, 1238, 3662, 561, 562, 1058, 4203, 1899, 3981, 1062, 1303, 4116, 567, 1946, 3620, 1514, 4002, 3679, 3964, 3707, 3666, 1348, 4273, 1804, 4497, 1401, 4438, 1895, 1264, 572, 1912, 3939, 4170, 4056, 1271, 1065, 1521, 1221, 1451, 1654, 3934, 4080, 4352, 1700, 3739, 1565, 1419, 1811, 3950, 1690, 1397, 3716, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 1723, 1070, 3750, 3633, 4070, 4104, 4162, 4235, 1863, 4346, 1881, 1814, 1572, 4443, 3761, 1346, 1587, 1072, 3592, 576, 1891, 4469, 1275, 1950, 1963, 4129, 1296, 1551, 3733, 4111, 3993, 4108, 3949, 1287, 1713, 1736, 1995, 1435, 1424, 1830, 1705, 1861, 1197, 1254, 4120, 1545, 4417, 1469, 1785, 1236, 4293, 3839, 3983, 4395, 1957, 3928, 4295, 3715, 3855, 3609, 1990, 4076, 4175, 3927, 1757, 1650, 4299, 1773, 2004, 1267, 1364, 1462, 1879, 4007, 3729, 3665, 3722, 1691, 1624, 1751, 1394, 3924, 3952, 580, 581, 2003, 1343, 1559, 1250, 1555, 4114, 1818, 1075, 1076, 1077, 1704, 4448, 1078, 1079, 1786, 585, 3354, 1832, 3432, 2891, 2505, 1903, 1081, 1082, 4459, 1083, 1563, 590, 591, 1085, 594, 1453, 2559, 2590, 2738, 4478, 4069, 1809, 1327, 4150, 3260, 3481, 3672, 3625, 2008, 4439, 3852, 3997, 1411, 599, 3847, 1268, 4050, 3377, 4424, 1496, 4143, 4493, 3492, 1962, 600, 601, 4017, 4477, 1794, 4263, 3072, 2009, 1754, 1087, 1088, 3888, 1368, 3827, 4103, 606, 1090, 1571, 1091, 1438, 1092, 1093, 612, 1094, 3701, 4389, 613, 1603, 1332, 1715, 4369, 1652, 1096, 617, 1541, 618, 1921, 1733, 1373, 1870, 619, 4218, 1798, 1100, 1702, 1101, 1667, 1384, 623, 3700, 1788, 1600, 624, 626, 2771, 627, 1339, 3875, 628, 629, 3070, 4860, 2557, 2795, 2906, 3118, 1614, 1947, 4479, 3766, 632, 1104, 1105, 633, 1813, 636, 3706, 1110, 640, 1111, 641, 642, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 3095, 1286, 3624, 4481, 1432, 3511, 2735, 4381, 3295, 1585, 1114, 1766, 3221, 2643, 2739, 3920, 644, 1116, 645, 646, 647, 1801, 4279, 3762, 1637, 1509, 1119, 2965, 2369, 2695, 3293, 650, 651, 1120, 652, 1121, 3264, 655, 658, 659, 4035, 2910, 3391, 2808, 3099, 2913, 3676, 2852, 3284, 2731, 1609, 3994, 1319, 4467, 1802, 1227, 1623, 662, 663, 664, 1124, 1125, 1128, 1129, 668, 669, 1130, 670, 1131, 1527, 1132, 671, 1772, 1135, 672, 1933, 1731, 1793, 675, 676, 3342, 1138, 1139, 677, 679, 1142, 1143, 681, 1145, 682, 685, 3822, 689, 690, 691, 1147, 1601, 3905, 694, 695, 1414, 1150, 1795, 1372, 1695, 1866, 4115, 1719, 698, 1920, 699, 700, 1544, 4305, 1636, 1393, 2010, 3810, 4452, 3677, 4227, 1277, 1400, 4402, 4157, 4197, 1337, 1253, 1532, 4028, 3989, 1493, 1854, 1902, 1510, 4182, 706, 707, 708, 4476, 1245, 714, 1841, 4234, 1314, 4096, 717, 1155, 4217, 1156, 723, 1157, 1627, 725, 1159, 1953, 1160, 1201, 727, 3787, 3747, 730, 1846, 3815, 2005, 1964, 1822, 1262, 4480, 1537, 1291, 1318, 4294, 1582, 734, 3439, 2833, 4287, 4509, 4505, 4267, 3697, 3272, 4112, 2967, 3651, 1628, 3705, 1195, 3859, 1164, 1165, 1166, 1745, 1973, 1168, 3807, 4307, 1454, or 1781.

27. The VP capsid polypeptide of any one of claims 20-24, wherein X⁶ of SEQ ID NO: 5014 is K or R.

28. The VP capsid polypeptide of claim 27, wherein the 581 to 589 region of the VP capsid polypeptide has a sequence of any one of SEQ ID NOs: 738, 4177, 1289, 740, 14, 743, 744, 17, 3477, 1548, 3051, 2982, 2877, 746, 19, 3036, 3426, 3073, 3169, 747, 3362, 2784, 21, 3192, 22, 1638, 748, 24, 749, 27, 1703, 1836, 28, 3660, 29, 750, 1640, 33, 35, 36, 1653, 1205, 3795, 753, 4288, 4219, 2770, 2962, 4241, 4107, 2718, 3317, 4391, 3263, 2553, 3947, 2710, 2608, 3467, 2796, 2961, 2529, 3320, 2674, 2635, 3446, 3053, 3250, 3688, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 1467, 3327, 3178, 3006, 3252, 2991, 3470, 2888, 2572, 3418, 2642, 2519, 3363, 40, 2633, 44, 45, 756, 47, 48, 1952, 3122, 2551, 3718, 3484, 3577, 1308, 3960, 1378, 2994, 3080, 2589, 3681, 51, 760, 762, 3925, 3654, 763, 59, 60, 4086, 1740, 1660, 62, 1966, 63, 2438, 768, 66, 67, 68, 3816, 2599, 2706, 72, 73, 74, 3865, 1549, 1361, 3882, 773, 3713, 1942, 1312, 4501, 76, 1413, 775, 1560, 4289, 3757, 3593, 3908, 4397, 4415, 3723, 3754, 4360, 3945, 2854, 3523, 3669, 1356, 4065, 1294, 1484, 85, 86, 3891, 4456, 4040, 4496, 1982, 2001, 1478, 1189, 1490, 1885, 1926, 89, 780, 1180, 92, 781, 782, 783, 786, 94, 95, 788, 1831, 1867, 96, 4458, 97, 1970, 1936, 1590, 98, 99, 789, 790, 1298, 1688, 791, 792, 2007, 102, 103, 104, 105, 107, 108, 1579, 1906, 111, 798, 112, 799, 800, 1444, 801, 114, 115, 1344, 116, 802, 117, 118, 119, 121, 803, 1463, 1760, 806, 126, 128, 807, 129, 131, 808, 133, 1978, 811, 812, 135, 136, 137, 138, 814, 140, 1326, 141, 142, 143, 2344, 816, 1634, 1302, 817, 818, 147, 148, 149, 150, 151, 154, 155, 819, 820, 156, 157, 158, 161, 162, 165, 822, 168, 169, 170, 823, 172, 1651, 173, 1483, 174, 825, 176, 178, 180, 827, 181, 828, 4911, 184, 186, 188, 189, 830, 192, 193, 194, 196, 1391, 198, 199, 200, 201, 202, 203, 205, 832, 206, 834, 207, 208, 209, 210, 835, 836, 1334, 3901, 3312, 2649, 1450, 4386, 3028, 1446, 1522, 3490, 217, 219, 1612, 220, 221, 222, 839, 223, 840, 224, 225, 228, 229, 230, 231, 842, 232, 233, 234, 238, 847, 239, 850, 851, 853, 1536, 240, 241, 242, 243, 244, 245, 247, 250, 251, 254, 255, 857, 256, 258, 259, 859, 260, 861, 862, 864, 263, 2318, 865, 2326, 264, 1241, 266, 1477, 867, 268, 868, 269, 270, 869, 870, 271, 871, 872, 272, 873, 1252, 1591, 875, 274, 275, 276, 876, 877, 277, 278, 280, 1892, 1539, 881, 3601, 3877, 1460, 282, 1799, 284, 1626, 286, 1374, 1620, 1290, 1354, 887, 1956, 889, 292, 4324, 4138, 891, 296, 3903, 297, 3829, 3809, 4271, 3844, 1436, 300, 1617, 1961, 302, 304, 305, 2147, 1589, 4370, 307, 3646, 4059, 1431, 1331, 312, 1779, 313, 896, 1558, 1552, 318, 319, 322, 1958, 323, 324, 901, 1491, 328, 329, 4484, 907, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 3414, 3093, 2996, 3141, 3544, 3102, 2856, 2957, 3466, 3461, 2788, 2660, 3344, 3204, 2720, 3379, 2826, 1789, 2785, 3249, 3527, 4460, 3352, 2569, 4457, 2011, 4319, 3906, 1533, 1755, 4248, 1557, 3804, 1602, 4188, 1330, 4349, 3337, 3533, 2629, 3038, 2539, 3567, 3291, 3348, 3115, 3359, 2993, 3079, 3506, 2527, 2963, 2938, 2516, 3113, 2639, 3143, 3044, 2747, 3154, 3280, 3039, 2743, 2902, 3056, 2554, 2522, 3103, 3210, 3434, 2656, 4251, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 2869, 2610, 3298, 2775, 3108, 2603, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 2584, 2712, 3213, 3451, 2634, 3150, 2810, 3303, 2573, 3460, 2615, 3394, 2828, 3329, 4377, 4499, 3555, 2974, 3629, 3578, 3885, 2817, 3248, 2954, 3334, 3571, 3345, 3551, 3384, 3422, 2530, 2973, 4418, 4081, 3951, 1764, 1671, 1843, 3449, 3185, 3399, 2786, 3055, 3472, 3397, 2601, 3564, 3277, 3548, 3179, 3407, 3841, 3347, 3142, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3120, 3286, 2843, 3465, 3946, 2682, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 4269, 3487, 3489, 3019, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 1911, 908, 1935, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1464, 1886, 2666, 3246, 3130, 3560, 3236, 2730, 3572, 3689, 2968, 1457, 3450, 2577, 2567, 2532, 3415, 2934, 2984, 2568, 4147, 3002, 2986, 910, 2979, 2542, 1762, 4472, 1529, 4057, 1976, 4507, 1583, 1711, 2013, 1594, 4440, 344, 920, 921, 4498, 3728, 3637, 3570, 3429, 4091, 2870, 2995, 4338, 3205, 2880, 3261, 3968, 4291, 4204, 4071, 4034, 1742, 2820, 3912, 3287, 1218, 1190, 4145, 354, 1782, 927, 3610, 356, 4224, 1918, 1959, 1840, 3736, 1770, 929, 930, 931, 4155, 360, 361, 362, 364, 1279, 365, 1543, 366, 367, 4049, 1633, 1722, 369, 1495, 1566, 373, 1211, 1726, 1442, 1748, 4142, 1434, 1358, 1191, 3732, 4110, 4039, 4506, 4156, 4036, 4030, 4016, 3899, 1247, 1855, 3919, 1244, 4140, 3930, 4117, 4189, 4001, 3643, 3619, 1240, 1575, 3871, 4135, 4408, 3604, 1274, 4149, 3741, 1938, 3709, 3622, 3834, 1983, 1743, 3979, 4461, 4113, 3817, 4232, 4214, 3902, 1219, 1486, 1512, 1516, 4047, 4082, 4094, 1574, 1248, 4078, 1872, 1426, 3687, 4318, 4243, 3866, 3704, 4172, 1856, 3324, 2769, 2678, 1561, 3296, 377, 1395, 1584, 4093, 4453, 4210, 4009, 4264, 4339, 1738, 4474, 4013, 4266, 1771, 1759, 1922, 3860, 3832, 1349, 1381, 3663, 1185, 3932, 3858, 1198, 4192, 3611, 1919, 3790, 3712, 4095, 1887, 4468, 1720, 4247, 934, 1790, 2002, 1657, 1336, 3894, 3576, 4314, 3695, 1850, 4362, 1815, 3638, 4454, 1661, 4504, 1359, 3650, 1305, 1648, 3831, 4435, 4451, 1993, 3849, 1718, 4068, 1835, 1425, 1428, 4380, 1658, 1452, 4024, 4072, 3791, 4491, 4323, 4014, 1304, 1511, 1981, 1676, 4422, 1283, 3861, 3644, 4259, 1418, 1176, 4216, 1421, 3992, 4423, 4253, 3748, 4119, 1297, 1954, 4450, 3711, 4344, 1578, 4384, 3892, 3674, 3595, 4405, 3973, 4109, 4159, 3744, 4387, 4275, 4492, 4365, 4118, 4278, 3751, 3678, 4353, 3175, 2970, 1228, 3534, 3437, 3406, 3641, 4470, 3941, 4348, 3587, 3818, 4350, 3821, 3929, 3824, 4465, 1398, 3828, 1284, 3773, 4209, 3014, 3884, 2783, 4038, 3545, 2916, 3112, 2834, 3880, 4399, 4048, 3752, 4207, 1235, 3943, 4355, 4087, 4100, 4163, 4486, 4178, 1643, 4152, 4445, 4303, 4284, 1213, 4382, 1399, 3714, 4487, 3615, 4018, 4420, 4121, 3724, 4282, 4310, 4488, 4127, 3670, 4052, 3627, 3777, 1371, 3685, 4258, 3812, 1581, 4124, 4508, 4500, 3652, 3972, 1528, 3717, 2740, 3125, 3546, 3842, 4404, 3690, 3962, 4136, 3589, 3907, 4153, 2707, 4180, 3923, 4444, 4361, 4394, 3758, 4510, 3742, 1710, 1407, 4187, 1193, 1188, 1196, 1724, 1717, 1200, 1997, 1701, 4280, 4006, 1984, 3833, 2561, 4442, 3900, 4298, 1761, 4388, 1597, 4317, 4249, 1341, 1203, 1680, 4441, 3628, 3725, 1945, 937, 4206, 1588, 1439, 3794, 4447, 3776, 3658, 4064, 4011, 4272, 4503, 1324, 3594, 3680, 1300, 1750, 3913, 3774, 1877, 1987, 3998, 3686, 3696, 4463, 3853, 4244, 4083, 1909, 3897, 1546, 3613, 3273, 3452, 3525, 1229, 3835, 1234, 3970, 3856, 3586, 1175, 4321, 2778, 4412, 4407, 3823, 1427, 1507, 1878, 4333, 3146, 4281, 4211, 3843, 1677, 1524, 1525, 1974, 1239, 4167, 3740, 1342, 1471, 4176, 1232, 1989, 4125, 1741, 1580, 4060, 1576, 1212, 3921, 1174, 4061, 4021, 3693, 3734, 1382, 3684, 3806, 3635, 3976, 3840, 4286, 3836, 4161, 3659, 3605, 4392, 4328, 1316, 4173, 4265, 1246, 1629, 1829, 3826, 1992, 1656, 1914, 1839, 3984, 3910, 4160, 4184, 4132, 4250, 1792, 3769, 1534, 4003, 1689, 1599, 1265, 1410, 1823, 1498, 1329, 386, 940, 1655, 3874, 4261, 3626, 3579, 1644, 3936, 4054, 4260, 4297, 4357, 4022, 4046, 1222, 1996, 3788, 3953, 4226, 4277, 4101, 3797, 4019, 3771, 3632, 1402, 4137, 3772, 3661, 4186, 4174, 1170, 3850, 1315, 1611, 1859, 4183, 1403, 4033, 3623, 3596, 1461, 1848, 1173, 1928, 1765, 3719, 3584, 4165, 4191, 3980, 4058, 4200, 4336, 3958, 1260, 4051, 3798, 4032, 1774, 4322, 3991, 4106, 3978, 3965, 4464, 3813, 4154, 1365, 3846, 4345, 4379, 3603, 3937, 3801, 1787, 1214, 4483, 4201, 1845, 1220, 4245, 1347, 3867, 1900, 3640, 3916, 4089, 3745, 3975, 4256, 4308, 4000, 3868, 4403, 3883, 4326, 4393, 3895, 4237, 3955, 4005, 4092, 4372, 4283, 3805, 4151, 3982, 3597, 3872, 3987, 4490, 3876, 3668, 4300, 1679, 1242, 3598, 3583, 3647, 4304, 3779, 1907, 4042, 3648, 3996, 3800, 4367, 4099, 1807, 3887, 4428, 1625, 1390, 1884, 1682, 4364, 4306, 3869, 1278, 942, 1505, 1709, 3600, 3914, 3948, 1865, 1716, 3961, 1506, 4221, 1376, 1683, 4276, 1721, 1632, 3607, 1455, 1913, 1497, 1317, 1951, 1778, 3768, 4225, 1668, 390, 4406, 4341, 1480, 1714, 1422, 397, 3618, 4433, 1177, 3898, 398, 950, 400, 1965, 4471, 953, 401, 403, 955, 1482, 3602, 4434, 3721, 1592, 1825, 1698, 1519, 1352, 4409, 1977, 405, 1631, 1684, 409, 3889, 2117, 411, 413, 414, 415, 961, 1905, 416, 963, 3675, 1833, 3974, 965, 970, 1897, 1272, 4133, 3830, 972, 424, 427, 1708, 4141, 3749, 1619, 975, 4044, 3590, 1944, 4126, 1923, 1706, 432, 976, 978, 4400, 1606, 980, 2604, 2895, 436, 3448, 2188, 438, 1692, 442, 3862, 4055, 4358, 2276, 985, 1664, 443, 4309, 4045, 3854, 3682, 1972, 2688, 4366, 3425, 3231, 3649, 2759, 2724, 3755, 1216, 3599, 3775, 2754, 2606, 3471, 3234, 2565, 2803, 2562, 2684, 3411, 3405, 2690, 1255, 3694, 3985, 3535, 1306, 4084, 3282, 1780, 448, 990, 991, 1485, 992, 1728, 993, 994, 1696, 997, 3116, 1869, 456, 1386, 1000, 3879, 2698, 460, 1001, 461, 1447, 1002, 1003, 463, 464, 4425, 465, 1004, 466, 469, 1231, 1468, 470, 1005, 476, 1273, 1744, 1488, 1659, 1604, 479, 1013, 1014, 481, 4313, 1499, 1016, 4073, 1777, 485, 4274, 487, 1775, 4205, 1526, 1697, 2000, 488, 1894, 1882, 1817, 1880, 490, 1018, 3917, 3616, 4311, 4473, 4085, 4401, 3783, 3765, 1538, 3870, 491, 3699, 3792, 4290, 494, 2421, 4090, 2012, 1803, 3642, 1322, 1020, 502, 1333, 504, 1890, 507, 3608, 1732, 1864, 1816, 1531, 513, 3636, 514, 1699, 1027, 1366, 4950, 4027, 3478, 2959, 1293, 3197, 1595, 3413, 518, 1032, 1033, 1675, 1797, 2315, 521, 1999, 3759, 1215, 4122, 1730, 3966, 1035, 4973, 528, 3911, 3289, 1821, 530, 532, 1874, 533, 1038, 538, 539, 3767, 1929, 3683, 1338, 1616, 1901, 1796, 1517, 4331, 4332, 4466, 2719, 4123, 4233, 1824, 3129, 3087, 3505, 4158, 4254, 3720, 4351, 4414, 3453, 3254, 1649, 544, 4240, 3580, 1046, 548, 549, 2598, 552, 553, 3819, 1259, 4062, 554, 2841, 3515, 1433, 1323, 4301, 555, 558, 3738, 1567, 559, 1054, 1270, 3857, 2175, 1257, 1238, 3662, 561, 1058, 563, 1061, 4203, 1899, 3981, 1062, 1303, 4116, 1946, 3620, 1514, 4002, 3679, 3964, 3707, 3666, 1348, 4273, 1804, 4497, 1401, 4438, 1895, 1264, 1912, 573, 3939, 4170, 4056, 1271, 1521, 1221, 1451, 3934, 4080, 4352, 1700, 3739, 1419, 1811, 3950, 1690, 3716, 1569, 1437, 1674, 4337, 1285, 4268, 1183, 3881, 3938, 4198, 4416, 4229, 4292, 4193, 4325, 4375, 4432, 4215, 3944, 4195, 4223, 1540, 4455, 4512, 1723, 3750, 4070, 4104, 4235, 1863, 4346, 1881, 1814, 4443, 3761, 1587, 3592, 576, 4469, 1275, 1950, 4129, 1296, 1551, 3733, 4111, 3993, 4108, 1287, 1713, 1556, 1736, 1995, 1705, 1861, 4120, 4417, 1785, 1236, 4293, 1827, 3839, 3983, 4395, 1957, 3928, 4295, 3715, 3855, 3609, 4076, 4175, 3927, 1757, 4299, 1773, 1364, 1462, 4007, 3729, 3665, 3722, 1691, 1712, 1624, 1751, 1394, 3924, 3952, 581, 2003, 1559, 1250, 1555, 4114, 1818, 1076, 4448, 1078, 1786, 3354, 1832, 3432, 2891, 1903, 4459, 589, 1563, 1084, 591, 592, 594, 595, 1858, 596, 1453, 2559, 2590, 2738, 4478, 4069, 1809, 1327, 4150, 3260, 3481, 3672, 3625, 4439, 3852, 3997, 3847, 4050, 3377, 4424, 4143, 4493, 3492, 1412, 4017, 4477, 1794, 602, 4263, 3072, 2009, 1754, 3888, 603, 604, 1368, 3827, 4103, 1571, 1438, 611, 1093, 612, 1094, 3701, 4389, 614, 615, 1332, 1715, 4369, 1652, 1541, 1921, 1733, 1373, 1870, 619, 4218, 1798, 1702, 1384, 3700, 1788, 1600, 624, 2473, 1423, 2771, 1339, 3875, 3070, 2015, 2557, 2795, 2906, 2103, 3299, 3118, 4576, 1103, 1614, 1947, 631, 4479, 3766, 1107, 635, 1813, 636, 2431, 637, 1109, 639, 3706, 1110, 640, 642, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 1269, 3095, 1286, 3624, 4481, 3511, 2735, 4381, 3295, 1568, 1766, 3221, 2643, 2739, 3920, 647, 1801, 4279, 3762, 1637, 1509, 648, 1117, 1119, 2965, 2695, 3293, 4722, 1121, 653, 1122, 3264, 655, 656, 657, 4035, 2910, 3391, 2808, 3099, 2913, 3676, 2852, 3284, 2731, 3537, 3994, 1319, 4467, 1623, 660, 1123, 664, 1126, 1128, 1129, 1130, 670, 1527, 671, 1772, 1137, 5005, 1933, 1731, 1793, 3342, 1140, 1141, 687, 3822, 1601, 1148, 693, 3905, 694, 1414, 1149, 697, 1795, 1372, 1695, 1866, 1151, 4115, 1719, 1920, 4999, 1544, 703, 4031, 4305, 1636, 3810, 4452, 3677, 4227, 4402, 4157, 4197, 1337, 1253, 4028, 3989, 704, 1493, 1902, 1152, 4182, 707, 708, 709, 4476, 1375, 713, 714, 1841, 4234, 1154, 1314, 4096, 718, 719, 720, 2063, 1311, 721, 4217, 1627, 1158, 1953, 1201, 3787, 729, 3747, 730, 1846, 3815, 1964, 1822, 1262, 4480, 1537, 1291, 1318, 4294, 1582, 3439, 2833, 4287, 4509, 4505, 4267, 3697, 3272, 4112, 2967, 3651, 1805, 3705, 1195, 3859, 1164, 1165, 2351, 2423, 1745, 1973, 3807, 4307, 1169, 1454, or 1781.

29. The VP capsid polypeptide of any one of claims 1-28, wherein:
(a) X³ of SEQ ID NO: 5014 is not D,
(b) X⁶ of SEQ ID NO: 5014 is not D, or
(c) X³ of SEQ ID NO: 5014 is not D and X⁶ of SEQ ID NO: 5014 is not D.

30. The VP capsid polypeptide of claim 29, wherein the 581 to 589 region of the VP capsid polypeptide has a sequence of any one of SEQ ID NOs: 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 2469, 4889, 21, 3192, 4942, 22, 23, 1638, 748, 24, 25, 749, 26, 27, 1703, 1836, 28, 2038, 2301, 3660, 29, 750, 30, 31, 32, 2095, 1640, 33, 34, 35, 36, 37, 4742, 2450, 38, 4925, 39, 751, 752, 2144, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2080, 2072, 2034, 50, 1308, 1230, 3960, 1378, 2994, 3080, 2589, 3681, 51, 2167, 52, 760, 53, 2389, 4755, 761, 4591, 762, 4659, 54, 2146, 3925, 55, 3654, 2087, 2346, 4766, 4682, 4984, 4744, 56, 763, 764, 4626, 4702, 57, 58, 59, 60, 61, 4086, 1740, 1660, 765, 766, 62, 1966, 2032, 2499, 2332, 767, 63, 2096, 2181, 4913, 2438, 2041, 64, 768, 769, 770, 2384, 2402, 771, 65, 66, 2111, 67, 772, 2458, 2197, 68, 3816, 69, 2599, 2706, 2497, 70, 2399, 71, 4769, 2366, 72, 73, 74, 3865, 1549, 1361, 2453, 3882, 773, 3713, 1942, 2140, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2379, 81, 82, 4065, 83, 84, 2030, 1294, 2413, 2064, 1484, 2299, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 93, 2356, 784, 785, 786, 94, 95, 788, 1831, 1867, 96, 4458, 97, 1970, 4656, 2173, 1936, 2349, 1590, 98, 99, 100, 789, 790, 1298, 101, 1688, 791, 792, 2007, 4556, 2070, 102, 793, 4632, 103, 794, 795, 104, 105, 106, 2229, 796, 4980, 4691, 107, 2440, 1849, 108, 2014, 797, 4736, 2509, 2018, 2227, 4787, 1579, 110, 1906, 111, 2428, 798, 2075, 112, 2411, 113, 2359, 799, 4848, 800, 2461, 1444, 2313, 2350, 4579, 4821, 801, 114, 2329, 4864, 4834, 115, 1344, 116, 802, 117, 118, 119, 4929, 4677, 120, 4791, 2158, 121, 803, 804, 1463, 805, 122, 2139, 2290, 1760, 2465, 123, 2424, 2058, 2230, 124, 4717, 806, 125, 126, 2370, 127, 4521, 128, 4971, 4994, 807, 2085, 4533, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 4544, 5009, 144, 145, 816, 4654, 2125, 4663, 4810, 4816, 1634, 1302, 817, 146, 818, 147, 148, 149, 4546, 150, 151, 152, 153, 154, 155, 819, 820, 156, 2149, 157, 2295, 821, 158, 159, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 2170, 4809, 4520, 180, 826, 827, 181, 828, 4695, 2130, 4911, 182, 4582, 4873, 2189, 183, 4641, 829, 184, 185, 2244, 4647, 186, 187, 188, 189, 190, 2114, 830, 191, 192, 193, 194, 2071, 4615, 2361, 195, 4852, 2187, 4914, 4947, 196, 1391, 197, 4534, 4726, 4788, 198, 199, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2090, 2241, 217, 218, 4653, 219, 1612, 220, 221, 222, 839, 223, 4974, 4790, 4915, 2464, 4567, 4774, 4964, 2360, 840, 224, 4928, 4741, 225, 2088, 4751, 226, 227, 4890, 4519, 4883, 228, 4786, 229, 230, 231, 2393, 2448, 841, 842, 232, 233, 4782, 234, 2269, 843, 235, 844, 236, 237, 4930, 845, 846, 238, 847, 239, 848, 849, 2150, 850, 851, 852, 853, 1536, 240, 854, 241, 242, 243, 244, 245, 246, 247, 248, 4696, 855, 4634, 856, 249, 250, 251, 252, 253, 254, 255, 857, 2115, 4880, 2218, 256, 257, 258, 259, 858, 859, 260, 261, 860, 861, 862, 262, 4966, 863, 864, 263, 4749, 4970, 2296, 2318, 4535, 865, 2326, 264, 1241, 265, 266, 866, 1477, 267, 867, 4977, 268, 868, 269, 4668, 5002, 270, 869, 870, 271, 871, 872, 272, 4822, 4709, 873, 4655, 4972, 2392, 2317, 874, 1252, 273, 1591, 4975, 875, 274, 2025, 275, 1980, 276, 876, 877, 2066, 277, 278, 279, 4670, 280, 2323, 2288, 2334, 4939, 4689, 281, 878, 2416, 1892, 879, 1539, 2214, 2215, 880, 2327, 881, 882, 4601, 883, 1295, 3601, 3877, 1930, 1420, 1678, 1460, 2306, 884, 282, 1799, 283, 284, 4988, 4871, 4553, 4772, 4817, 2260, 1626, 2082, 885, 286, 1374, 886, 2119, 2504, 2468, 287, 1620, 1290, 2425, 2482, 4564, 4671, 2282, 288, 2354, 1354, 2033, 289, 887, 290, 888, 291, 1956, 2362, 2467, 889, 2357, 4547, 4765, 292, 4794, 2391, 4324, 293, 890, 294, 295, 4138, 891, 2297, 2254, 296, 2224, 3903, 297, 892, 3829, 298, 299, 3809, 4271, 3844, 1436, 300, 1515, 1617, 301, 1961, 4649, 5010, 302, 2060, 303, 304, 305, 2452, 2147, 4574, 4538, 306, 2293, 1589, 893, 4946, 4370, 4841, 2342, 307, 308, 309, 894, 2246, 2155, 3646, 4059, 1431, 1331, 310, 311, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 904, 1635, 4820, 905, 2506, 331, 4706, 4484, 1756, 906, 907, 4661, 2179, 4584, 4609, 332, 4907, 4635, 4529, 4683, 4523, 4561, 333, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4319, 3906, 1533, 1755, 1888, 4248, 1557, 2153, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 2363, 908, 1935, 4711, 4728, 2460, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 4943, 3002, 2986, 910, 2979, 2542, 2338, 2331, 2492, 1762, 4472, 1472, 1529, 2319, 2131, 4057, 4937, 2048, 4887, 1976, 4507, 1583, 338, 2078, 2372, 2102, 339, 911, 1711, 340, 912, 2013, 913, 2277, 914, 2403, 341, 915, 4672, 4902, 916, 342, 1594, 4440, 917, 343, 344, 4559, 918, 2439, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 371, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2345, 4471, 952, 953, 401, 402, 2475, 403, 404, 4758, 954, 955, 2437, 4139, 1482, 3602, 4434, 3721, 1592, 1825, 1698, 1519, 1352, 4202, 4409, 1977, 1917, 405, 406, 407, 408, 956, 957, 2267, 1631, 1684, 409, 2054, 3889, 958, 410, 2117, 411, 959, 412, 960, 2443, 4712, 4619, 2285, 413, 414, 415, 961, 2352, 2417, 962, 1905, 416, 963, 417, 418, 2382, 3675, 1833, 3974, 419, 964, 420, 421, 965, 966, 967, 4919, 968, 2365, 969, 970, 422, 1897, 2056, 2309, 1272, 4754, 2207, 971, 423, 4133, 3830, 4865, 972, 424, 973, 425, 2068, 4955, 426, 974, 2109, 2380, 427, 1708, 4676, 428, 4141, 429, 430, 3749, 431, 1619, 975, 4044, 3590, 1944, 4126, 1923, 1706, 432, 976, 977, 978, 4867, 4400, 433, 434, 1606, 2143, 979, 435, 980, 2604, 2895, 981, 436, 3448, 4959, 4563, 2188, 437, 438, 2055, 1692, 982, 2388, 2243, 983, 2035, 2120, 4940, 439, 440, 441, 2474, 4900, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 2242, 453, 2286, 992, 1728, 993, 4703, 994, 995, 454, 1696, 996, 2039, 455, 997, 4874, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 466, 2328, 467, 468, 469, 1231, 1468, 470, 471, 2496, 472, 473, 1005, 474, 475, 2108, 4725, 1006, 476, 1273, 1007, 1744, 1488, 1659, 477, 478, 4598, 1604, 4753, 2493, 479, 1008, 1009, 480, 1010, 1011, 1012, 1013, 1014, 481, 4313, 482, 483, 1499, 484, 1015, 4761, 2091, 2386, 2513, 1016, 4073, 1777, 2172, 485, 2205, 4592, 4545, 4720, 486, 1458, 4274, 2122, 4905, 4796, 2152, 4539, 4528, 4904, 4842, 4986, 4639, 4846, 4623, 4650, 4595, 2195, 4681, 487, 2182, 4710, 1775, 2083, 4205, 1526, 4586, 1697, 1017, 2000, 4692, 4795, 2480, 2017, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 5001, 2136, 493, 2211, 4532, 494, 2421, 495, 4090, 2378, 2104, 2210, 2247, 2478, 2047, 496, 4620, 4978, 4614, 1019, 2012, 1803, 3642, 497, 1322, 4752, 1020, 498, 1021, 2235, 2302, 2488, 4665, 4699, 4608, 4781, 499, 2049, 2404, 4685, 500, 501, 1022, 502, 1333, 503, 1023, 4686, 4804, 504, 4727, 505, 506, 4938, 4805, 2065, 1890, 4891, 1024, 507, 508, 509, 2110, 1025, 510, 1026, 3608, 1732, 511, 2397, 1864, 1816, 1531, 512, 513, 2451, 3636, 5012, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 2093, 4746, 2489, 4922, 2145, 1030, 4832, 4950, 1261, 4027, 3478, 3763, 2959, 1293, 2462, 517, 3197, 1595, 3413, 2067, 1031, 2228, 1448, 518, 4698, 4616, 2057, 2305, 2133, 2061, 4526, 4617, 1032, 1033, 2429, 2311, 1675, 1797, 519, 4578, 2271, 4693, 4604, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 535, 1038, 536, 1039, 537, 538, 539, 1040, 3767, 1929, 1041, 1042, 3683, 2100, 4853, 2300, 2165, 540, 2105, 541, 1043, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1649, 543, 544, 545, 546, 1045, 547, 2225, 4240, 3580, 4680, 1046, 548, 549, 1047, 550, 2598, 551, 552, 553, 3819, 1259, 4062, 1048, 554, 2841, 1049, 1050, 3515, 2219, 1433, 1323, 4301, 555, 556, 1051, 557, 558, 1052, 1053, 3738, 1567, 559, 1054, 1270, 3857, 1055, 2151, 560, 2175, 2089, 1056, 4514, 2198, 2193, 1257, 1238, 3662, 561, 2186, 1057, 4637, 562, 1058, 563, 4872, 2097, 2436, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 575, 4443, 3761, 1346, 1587, 1072, 3592, 576, 1891, 4469, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2185, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 2304, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2113, 590, 591, 1085, 2256, 592, 2308, 593, 594, 2274, 595, 4931, 2407, 1858, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 2321, 1086, 601, 4017, 4477, 1794, 2483, 4776, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 2320, 2069, 610, 611, 1093, 612, 1094, 3701, 4389, 613, 4897, 1603, 614, 1095, 2154, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4855, 4847, 2398, 2239, 1101, 1667, 1384, 623, 4745, 3700, 1788, 1600, 624, 2098, 625, 4550, 4721, 4960, 4878, 2473, 4792, 4675, 1102, 1423, 626, 2771, 627, 1339, 3875, 628, 629, 3070, 4565, 2118, 2015, 4818, 4860, 2557, 2795, 2906, 2103, 4869, 2029, 3299, 3118, 4932, 4838, 4901, 4908, 4996, 4779, 2377, 4657, 4646, 4875, 4627, 4515, 4876, 4965, 4605, 4576, 2028, 4985, 4799, 4850, 1103, 4757, 4747, 1614, 1947, 2237, 630, 631, 4997, 4618, 4479, 3766, 632, 1104, 1105, 4886, 1106, 2432, 4827, 633, 1107, 4530, 634, 2371, 1108, 4536, 635, 1813, 636, 2431, 4866, 4991, 4814, 4560, 4956, 637, 4555, 1109, 4941, 4633, 2213, 638, 639, 3706, 1110, 640, 1111, 641, 4581, 4893, 4831, 2481, 4644, 4734, 2190, 4524, 4881, 4837, 4888, 4648, 642, 4548, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4629, 1115, 4918, 4688, 3920, 644, 4723, 4949, 4748, 4607, 4719, 1116, 645, 646, 4611, 2353, 4603, 4762, 4819, 2221, 4636, 4992, 4998, 2307, 647, 1801, 4279, 3762, 1637, 1509, 4843, 4797, 4590, 4516, 4995, 4541, 2200, 4580, 4858, 648, 649, 2502, 2477, 4724, 4551, 4674, 1118, 1119, 2965, 2369, 2695, 3293, 650, 651, 4730, 4862, 4722, 4944, 1120, 652, 1121, 4983, 4784, 4877, 4643, 653, 654, 4909, 1122, 3264, 655, 2076, 4849, 656, 657, 2447, 658, 659, 2191, 4035, 2910, 3391, 2808, 3099, 2913, 3676, 2852, 3284, 2731, 1609, 2375, 2201, 3537, 3994, 1319, 4467, 1802, 1227, 1623, 660, 4844, 4714, 4979, 4812, 2192, 1123, 661, 2364, 662, 663, 664, 1124, 1125, 665, 2487, 4899, 2138, 1126, 666, 667, 4569, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 674, 2456, 4588, 4963, 4851, 4558, 4557, 4697, 4610, 4773, 4687, 4708, 4910, 2123, 1136, 1137, 2358, 5005, 2208, 4952, 4945, 1933, 1731, 1793, 675, 676, 3342, 1138, 1139, 677, 4840, 4597, 4870, 1140, 4990, 4954, 4916, 678, 1141, 4737, 4527, 2253, 4531, 679, 1142, 1143, 680, 4771, 1144, 4638, 681, 4705, 4525, 4522, 4898, 2325, 2415, 5004, 1145, 682, 683, 2141, 1146, 684, 685, 2445, 686, 687, 4861, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4690, 4859, 4793, 4575, 2160, 4828, 4731, 4718, 4802, 4750, 1150, 2081, 2258, 697, 1795, 1372, 1695, 1866, 1151, 4115, 1719, 2280, 4662, 698, 2176, 2217, 1920, 699, 700, 701, 4999, 1544, 4739, 2512, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 2340, 4182, 706, 4652, 2442, 2484, 707, 708, 709, 4667, 1153, 2412, 4732, 4476, 2053, 710, 1375, 711, 712, 713, 4829, 4738, 4811, 4570, 1245, 714, 2045, 4707, 715, 2169, 4713, 1841, 4234, 4924, 1154, 1314, 4096, 2023, 717, 4895, 2414, 718, 1155, 719, 720, 2063, 1311, 4921, 721, 4833, 4903, 4885, 4666, 2249, 4217, 1156, 723, 1157, 1627, 2116, 4566, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 2248, 730, 731, 1846, 2507, 2368, 1162, 2019, 2333, 3815, 2005, 2266, 1964, 2347, 732, 2177, 2059, 4958, 4927, 1822, 4815, 1262, 2094, 4480, 1537, 1291, 2419, 1318, 4628, 4294, 2486, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4679, 4835, 2259, 4839, 2275, 1166, 4968, 4912, 2423, 737, 1167, 4989, 4684, 4612, 2127, 1745, 1973, 4767, 1168, 2273, 2310, 3807, 4307, 1169, 2303, 1454, or 1781.

31. The VP capsid polypeptide of claim 29, wherein the 581 to 589 region of the VP capsid polypeptide has a sequence of any one of SEQ ID NOs: 2498, 4884, 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 2469, 4889, 21, 3192, 4942, 22, 23, 1638, 748, 24, 25, 749, 26, 27, 1703, 1836, 28, 2038, 2301, 3660, 29, 750, 30, 31, 2095, 1640, 33, 34, 35, 36, 37, 4742, 2450, 38, 4925, 39, 751, 752, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2072, 2034, 50, 1308, 1230, 3960, 1378, 2994, 3080, 2589, 3681, 51, 4694, 2257, 2167, 52, 760, 53, 2389, 4934, 4951, 4755, 761, 4591, 762, 4659, 54, 2146, 3925, 55, 3654, 2087, 2346, 4766, 4682, 4984, 4744, 56, 763, 764, 4626, 4702, 57, 58, 59, 60, 61, 4086, 1740, 1660, 765, 766, 62, 1966, 2032, 2499, 2332, 767, 63, 2096, 2181, 4913, 2438, 2041, 64, 768, 769, 770, 2384, 2402, 771, 65, 66, 67, 772, 2458, 2197, 68, 3816, 69, 2599, 2706, 2497, 70, 2234, 2183, 2399, 71, 4769, 2366, 72, 73, 74, 3865, 1549, 1361, 2453, 3882, 773, 3713, 1942, 2140, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2132, 81, 82, 4065, 83, 84, 2030, 1294, 2413, 2064, 1484, 2299, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 2356, 784, 785, 786, 94, 95, 787, 788, 1831, 1867, 96, 4458, 97, 1970, 4656, 2173, 1936, 2349, 1590, 4585, 98, 99, 100, 789, 790, 1298, 101, 1688, 791, 792, 2007, 4556, 2070, 102, 793, 4632, 103, 794, 795, 104, 105, 106, 2229, 796, 4691, 107, 2440, 1849, 108, 797, 4736, 2509, 2018, 2227, 4787, 109, 1579, 110, 1906, 111, 2428, 798, 2075, 112, 2411, 113, 2359, 799, 4848, 800, 2461, 1444, 2313, 2350, 4579, 4821, 801, 114, 2329, 4864, 4834, 115, 1344, 116, 802, 117, 118, 119, 4929, 4677, 120, 4791, 2158, 121, 803, 804, 1463, 805, 122, 2139, 2290, 1760, 2465, 123, 2424, 2058, 2230, 124, 4717, 806, 125, 126, 2370, 127, 4521, 128, 4971, 4994, 807, 4533, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 5009, 144, 145, 816, 4654, 2125, 4663, 4810, 4816, 1634, 1302, 817, 146, 818, 147, 148, 149, 4546, 150, 151, 152, 153, 154, 155, 819, 820, 156, 2149, 157, 2295, 821, 158, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 4809, 4520, 180, 826, 827, 181, 828, 4695, 2130, 4911, 182, 4582, 4873, 4596, 2020, 2189, 183, 4641, 829, 184, 185, 2244, 4647, 186, 187, 188, 189, 190, 830, 191, 192, 193, 194, 2071, 4615, 2361, 195, 4852, 4914, 4947, 196, 1391, 197, 4534, 4726, 198, 199, 4920, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2090, 2241, 217, 218, 4653, 219, 1612, 220, 221, 222, 839, 223, 4953, 4974, 4790, 4915, 2464, 4567, 4774, 4964, 2360, 840, 224, 4928, 4741, 225, 2088, 4751, 5007, 4542, 226, 227, 4890, 4519, 4883, 228, 4786, 229, 230, 231, 2448, 841, 842, 232, 233, 4782, 234, 4962, 2269, 843, 235, 844, 236, 237, 4930, 845, 846, 238, 847, 4704, 239, 848, 849, 2150, 850, 851, 852, 853, 1536, 240, 854, 241, 242, 243, 244, 245, 246, 247, 248, 4923, 2245, 4696, 855, 4634, 856, 249, 250, 251, 252, 253, 254, 255, 857, 2115, 2218, 256, 257, 258, 259, 858, 859, 260, 261, 860, 861, 862, 262, 4966, 863, 864, 263, 4749, 4970, 2296, 2318, 4535, 865, 2326, 264, 1241, 265, 266, 866, 1477, 267, 867, 268, 868, 269, 4668, 270, 869, 870, 271, 871, 872, 272, 4822, 4709, 873, 4655, 4972, 2317, 874, 1252, 273, 1591, 4975, 875, 274, 2025, 275, 1980, 276, 876, 877, 2066, 277, 278, 279, 4670, 280, 2323, 2288, 2334, 4939, 4689, 281, 878, 2416, 1892, 879, 1539, 2214, 2215, 880, 2327, 881, 882, 4601, 1295, 3601, 3877, 1930, 1420, 1678, 1460, 2306, 884, 282, 1799, 283, 284, 4988, 4871, 4553, 4772, 2260, 285, 1626, 885, 286, 1374, 886, 2119, 2504, 2468, 287, 2408, 1620, 1290, 2425, 2482, 4564, 4671, 2282, 288, 1354, 2033, 289, 887, 290, 888, 291, 1956, 2362, 2467, 889, 2357, 4547, 4765, 292, 4794, 2391, 4324, 293, 890, 294, 295, 4138, 891, 2297, 2254, 296, 2224, 3903, 297, 892, 3829, 298, 299, 3809, 4271, 3844, 1436, 300, 1515, 1617, 4701, 2174, 301, 1961, 4649, 5010, 302, 2060, 303, 304, 305, 2452, 2147, 4574, 4538, 306, 2293, 1589, 893, 4946, 4370, 4841, 2342, 307, 308, 309, 894, 2246, 2155, 3646, 4059, 1431, 1331, 311, 4836, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 1635, 4820, 905, 331, 4706, 2092, 4484, 1756, 906, 907, 4661, 2179, 4584, 4609, 332, 4907, 4635, 4529, 4683, 4561, 333, 2337, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4783, 4319, 3906, 1533, 1755, 1888, 4248, 1557, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 2444, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 2363, 908, 1935, 4711, 4728, 2460, 4517, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 337, 1184, 3450, 2577, 2567, 2532, 3415, 2934, 2984, 2568, 4147, 4943, 3002, 2986, 910, 2979, 2542, 2338, 2331, 2492, 1762, 4472, 1472, 1529, 2319, 4057, 4937, 2048, 4887, 1976, 4507, 1583, 338, 2078, 2372, 2102, 339, 911, 1711, 340, 912, 2013, 913, 2277, 914, 2403, 341, 915, 4902, 916, 342, 1594, 4440, 917, 343, 344, 4559, 918, 2233, 2439, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 2471, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 371, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2343, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 2383, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2129, 2345, 4471, 952, 953, 401, 2475, 403, 404, 4758, 954, 955, 2437, 4139, 1482, 3602, 4434, 3721, 1592, 1825, 1698, 1519, 1352, 4202, 4409, 1977, 1917, 405, 406, 407, 408, 956, 2272, 957, 2267, 1631, 1684, 409, 2054, 3889, 958, 410, 2117, 411, 959, 412, 960, 2443, 4712, 4619, 2285, 413, 414, 415, 961, 2352, 2417, 962, 1905, 416, 963, 417, 418, 2382, 3675, 1833, 3974, 419, 964, 420, 421, 965, 966, 967, 968, 2365, 969, 970, 422, 1897, 2056, 2309, 4733, 1272, 4754, 2207, 971, 423, 4133, 3830, 4865, 972, 424, 973, 425, 4955, 426, 974, 2109, 2380, 427, 1708, 4676, 428, 4141, 429, 430, 3749, 431, 1619, 975, 4044, 3590, 1944, 4126, 1923, 1706, 432, 976, 977, 978, 4867, 4400, 433, 434, 1606, 979, 435, 980, 2604, 2895, 981, 436, 3448, 4959, 4563, 2188, 437, 438, 2055, 1692, 982, 2388, 2433, 2243, 983, 2035, 2120, 4940, 439, 440, 441, 2474, 442, 4900, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 2242, 453, 2286, 992, 1728, 993, 4703, 994, 995, 454, 1696, 996, 2039, 997, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 466, 2328, 467, 468, 469, 1231, 1468, 470, 471, 2496, 472, 473, 1005, 474, 2108, 4725, 1006, 476, 1273, 1007, 1744, 1488, 1659, 477, 478, 4598, 1604, 4753, 2493, 479, 1008, 1009, 1010, 1011, 1012, 1013, 1014, 481, 4313, 482, 483, 1499, 484, 1015, 4761, 2091, 2386, 2513, 1016, 4073, 1777, 2172, 485, 2205, 4592, 4545, 4720, 486, 1458, 4274, 2122, 4905, 5003, 4599, 4796, 2152, 4539, 4528, 4904, 4842, 4986, 4639, 4846, 4623, 4650, 4595, 2195, 4681, 2142, 487, 2182, 4710, 1775, 2083, 4205, 1526, 4586, 4602, 4518, 1697, 1017, 2000, 4692, 2480, 2017, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 2202, 5001, 2136, 493, 2211, 4532, 494, 2421, 495, 4090, 2378, 2210, 2247, 2478, 2047, 496, 4620, 4978, 1019, 2012, 1803, 3642, 497, 1322, 4752, 1020, 498, 1021, 2235, 2302, 2488, 4665, 4540, 5006, 4699, 4608, 4781, 499, 2049, 2404, 4685, 500, 501, 1022, 502, 1333, 503, 1023, 4686, 4804, 504, 4727, 505, 506, 4805, 2065, 1890, 1024, 507, 508, 509, 2110, 1025, 510, 1026, 3608, 1732, 511, 2397, 1864, 1816, 1531, 512, 513, 2451, 3636, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 4746, 2489, 4922, 2145, 1030, 4832, 4950, 1261, 4027, 3478, 3763, 2959, 1293, 2462, 517, 3197, 1595, 3413, 2238, 2067, 1031, 2228, 1448, 518, 4698, 4616, 2057, 2305, 2133, 2061, 4526, 4617, 1032, 1033, 2429, 2311, 1675, 4785, 1797, 519, 4578, 2271, 4693, 4604, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 1038, 536, 1039, 537, 538, 539, 1040, 3767, 1929, 1041, 1042, 3683, 2100, 4853, 2300, 2165, 540, 2105, 541, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1649, 543, 544, 545, 546, 1045, 547, 2084, 2225, 4240, 3580, 4680, 1046, 548, 2062, 549, 1047, 550, 2598, 551, 552, 553, 3819, 1259, 4062, 1048, 554, 2841, 1049, 1050, 3515, 1433, 1323, 4301, 555, 556, 1051, 557, 558, 1052, 1053, 3738, 1567, 559, 1054, 1270, 3857, 1055, 2151, 560, 2175, 2089, 4514, 2198, 2193, 1257, 1238, 3662, 561, 2186, 1057, 562, 1058, 563, 4872, 2097, 2436, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1705, 1861, 1197, 1254, 4120, 1545, 4417, 1469, 1292, 2185, 1785, 1236, 4293, 1827, 3839, 3983, 4395, 1957, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 2304, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2255, 2113, 590, 591, 1085, 2256, 592, 2308, 593, 594, 595, 4931, 2407, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 2321, 1086, 601, 4017, 4477, 1794, 2483, 4776, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 2320, 2069, 610, 611, 1093, 612, 1094, 3701, 4389, 613, 4897, 1603, 614, 1095, 2154, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4801, 4855, 4847, 2398, 2239, 1101, 1667, 1384, 623, 4745, 3700, 1788, 1600, 624, 2098, 625, 4550, 4721, 4960, 2473, 4792, 4675, 1102, 1423, 626, 2771, 627, 1339, 3875, 628, 629, 3070, 4565, 2118, 2015, 4818, 4860, 2557, 2795, 2906, 2103, 4869, 2029, 3299, 3118, 4932, 4838, 4901, 4908, 4996, 4779, 2377, 4657, 4646, 4875, 4627, 4515, 4876, 4965, 4605, 4576, 2028, 4985, 4799, 4850, 1103, 4757, 4747, 1614, 1947, 2237, 630, 631, 4997, 4618, 4479, 3766, 632, 1104, 1105, 4886, 1106, 2432, 4827, 633, 1107, 4530, 634, 2371, 4651, 1108, 4536, 635, 1813, 636, 2431, 4866, 4991, 4814, 4560, 4956, 637, 4555, 1109, 4577, 4633, 2213, 638, 639, 3706, 1110, 640, 1111, 641, 4581, 4893, 4831, 2481, 4644, 4734, 2190, 4881, 4837, 4888, 4648, 642, 4548, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4760, 4629, 1115, 4918, 4688, 3920, 644, 4723, 4949, 4607, 4719, 1116, 645, 646, 4611, 2353, 4645, 4603, 4762, 4819, 2221, 4636, 4992, 2307, 647, 1801, 4279, 3762, 1637, 1509, 4843, 4797, 4590, 4516, 4541, 2200, 4580, 4858, 648, 649, 1117, 4780, 4800, 2477, 4724, 4551, 4674, 1118, 1119, 2965, 2369, 2695, 3293, 650, 651, 4862, 4722, 4944, 1120, 652, 1121, 4983, 4784, 4877, 653, 654, 1122, 3264, 655, 2076, 4849, 656, 657, 2447, 658, 659, 2191, 4035, 2910, 3391, 2808, 3099, 2913, 3676, 2852, 3284, 2731, 1609, 2375, 3537, 3994, 1319, 4467, 1802, 1227, 1623, 660, 4844, 2086, 4770, 4714, 4979, 4812, 2192, 1123, 661, 2364, 662, 663, 664, 1124, 1125, 665, 2487, 4899, 2138, 1126, 666, 667, 4569, 4554, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 674, 2456, 4606, 4588, 4963, 4851, 4558, 4557, 4697, 4610, 4773, 4687, 4708, 4910, 2123, 1136, 1137, 2358, 5005, 2208, 4952, 4945, 1933, 1731, 1793, 675, 676, 3342, 1138, 1139, 677, 4840, 4597, 4870, 1140, 4990, 4954, 4916, 678, 1141, 4737, 4527, 2253, 4531, 679, 1142, 1143, 680, 4771, 1144, 4638, 681, 4705, 4525, 4898, 2325, 2415, 5004, 1145, 682, 683, 2141, 1146, 684, 685, 2445, 686, 687, 4861, 2157, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4690, 4859, 4793, 4575, 2160, 4828, 4731, 4802, 4750, 1150, 2081, 2258, 697, 1795, 1372, 1695, 1866, 1151, 4115, 1719, 2280, 4662, 698, 2176, 2194, 2217, 1920, 699, 700, 701, 4999, 1544, 4739, 2512, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 4824, 705, 2340, 4182, 706, 4652, 2442, 2484, 707, 708, 709, 4667, 1153, 2412, 4732, 4476, 2053, 710, 1375, 711, 712, 713, 4738, 4811, 4570, 4961, 1245, 714, 2045, 4707, 715, 2169, 4713, 4879, 1841, 4234, 4924, 1154, 1314, 4096, 2023, 716, 717, 4895, 2414, 718, 1155, 719, 720, 2063, 1311, 4921, 721, 4642, 722, 4833, 4903, 4885, 4666, 2249, 4217, 1156, 723, 1157, 1627, 2116, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 2248, 730, 731, 2024, 1846, 2507, 2368, 1162, 2019, 2333, 3815, 2005, 2266, 1964, 2347, 2385, 732, 2177, 2059, 4958, 4927, 1822, 4815, 1262, 2094, 4480, 1537, 1291, 2419, 1318, 2283, 4976, 4628, 4294, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4835, 2259, 4571, 2275, 1166, 4968, 2250, 2423, 737, 1167, 4989, 4669, 4552, 4612, 2127, 1745, 1973, 4767, 1168, 2273, 2310, 3807, 4307, 1169, 2303, 1454, or 1781.

32. The VP capsid polypeptide of claim 29, wherein the 581 to 589 region of the VP capsid polypeptide has a sequence of any one of SEQ ID NOs: 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 2469, 4889, 21, 3192, 4942, 22, 23, 1638, 748, 24, 25, 749, 26, 27, 1703, 1836, 28, 2038, 2301, 3660, 29, 750, 30, 31, 2095, 1640, 33, 34, 35, 36, 37, 4742, 2450, 38, 4925, 39, 751, 752, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2072, 2034, 50, 1308, 1230, 3960, 1378, 2994, 3080, 2589, 3681, 51, 2167, 52, 760, 53, 2389, 4755, 761, 4591, 762, 4659, 54, 2146, 3925, 55, 3654, 2087, 2346, 4766, 4682, 4984, 4744, 56, 763, 764, 4626, 4702, 57, 58, 59, 60, 61, 4086, 1740, 1660, 765, 766, 62, 1966, 2032, 2499, 2332, 767, 63, 2096, 2181, 4913, 2438, 2041, 64, 768, 769, 770, 2384, 2402, 771, 65, 66, 67, 772, 2458, 2197, 68, 3816, 69, 2599, 2706, 2497, 70, 2399, 71, 4769, 2366, 72, 73, 74, 3865, 1549, 1361, 2453, 3882, 773, 3713, 1942, 2140, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 81, 82, 4065, 83, 84, 2030, 1294, 2413, 2064, 1484, 2299, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 2356, 784, 785, 786, 94, 95, 788, 1831, 1867, 96, 4458, 97, 1970, 4656, 2173, 1936, 2349, 1590, 98, 99, 100, 789, 790, 1298, 101, 1688, 791, 792, 2007, 4556, 2070, 102, 793, 4632, 103, 794, 795, 104, 105, 106, 2229, 796, 4691, 107, 2440, 1849, 108, 797, 4736, 2509, 2018, 2227, 4787, 1579, 110, 1906, 111, 2428, 798, 2075, 112, 2411, 113, 2359, 799, 4848, 800, 2461, 1444, 2313, 2350, 4579, 4821, 801, 114, 2329, 4864, 4834, 115, 1344, 116, 802, 117, 118, 119, 4929, 4677, 120, 4791, 2158, 121, 803, 804, 1463, 805, 122, 2139, 2290, 1760, 2465, 123, 2424, 2058, 2230, 124, 4717, 806, 125, 126, 2370, 127, 4521, 128, 4971, 4994, 807, 4533, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 5009, 144, 145, 816, 4654, 2125, 4663, 4810, 4816, 1634, 1302, 817, 146, 818, 147, 148, 149, 4546, 150, 151, 152, 153, 154, 155, 819, 820, 156, 2149, 157, 2295, 821, 158, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 4809, 4520, 180, 826, 827, 181, 828, 4695, 2130, 4911, 182, 4582, 4873, 2189, 183, 4641, 829, 184, 185, 2244, 4647, 186, 187, 188, 189, 190, 830, 191, 192, 193, 194, 2071, 4615, 2361, 195, 4852, 4914, 4947, 196, 1391, 197, 4534, 4726, 198, 199, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2090, 2241, 217, 218, 4653, 219, 1612, 220, 221, 222, 839, 223, 4974, 4790, 4915, 2464, 4567, 4774, 4964, 2360, 840, 224, 4928, 4741, 225, 2088, 4751, 226, 227, 4890, 4519, 4883, 228, 4786, 229, 230, 231, 2448, 841, 842, 232, 233, 4782, 234, 2269, 843, 235, 844, 236, 237, 4930, 845, 846, 238, 847, 239, 848, 849, 2150, 850, 851, 852, 853, 1536, 240, 854, 241, 242, 243, 244, 245, 246, 247, 248, 4696, 855, 4634, 856, 249, 250, 251, 252, 253, 254, 255, 857, 2115, 2218, 256, 257, 258, 259, 858, 859, 260, 261, 860, 861, 862, 262, 4966, 863, 864, 263, 4749, 4970, 2296, 2318, 4535, 865, 2326, 264, 1241, 265, 266, 866, 1477, 267, 867, 268, 868, 269, 4668, 270, 869, 870, 271, 871, 872, 272, 4822, 4709, 873, 4655, 4972, 2317, 874, 1252, 273, 1591, 4975, 875, 274, 2025, 275, 1980, 276, 876, 877, 2066, 277, 278, 279, 4670, 280, 2323, 2288, 2334, 4939, 4689, 281, 878, 2416, 1892, 879, 1539, 2214, 2215, 880, 2327, 881, 882, 4601, 1295, 3601, 3877, 1930, 1420, 1678, 1460, 2306, 884, 282, 1799, 283, 284, 4988, 4871, 4553, 4772, 2260, 1626, 885, 286, 1374, 886, 2119, 2504, 2468, 287, 1620, 1290, 2425, 2482, 4564, 4671, 2282, 288, 1354, 2033, 289, 887, 290, 888, 291, 1956, 2362, 2467, 889, 2357, 4547, 4765, 292, 4794, 2391, 4324, 293, 890, 294, 295, 4138, 891, 2297, 2254, 296, 2224, 3903, 297, 892, 3829, 298, 299, 3809, 4271, 3844, 1436, 300, 1515, 1617, 301, 1961, 4649, 5010, 302, 2060, 303, 304, 305, 2452, 2147, 4574, 4538, 306, 2293, 1589, 893, 4946, 4370, 4841, 2342, 307, 308, 309, 894, 2246, 2155, 3646, 4059, 1431, 1331, 311, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 1635, 4820, 905, 331, 4706, 4484, 1756, 906, 907, 4661, 2179, 4584, 4609, 332, 4907, 4635, 4529, 4683, 4561, 333, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4319, 3906, 1533, 1755, 1888, 4248, 1557, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 2363, 908, 1935, 4711, 4728, 2460, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 337, 1184, 3450, 2577, 2567, 2532, 3415, 2934, 2984, 2568, 4147, 4943, 3002, 2986, 910, 2979, 2542, 2338, 2331, 2492, 1762, 4472, 1472, 1529, 2319, 4057, 4937, 2048, 4887, 1976, 4507, 1583, 338, 2078, 2372, 2102, 339, 911, 1711, 340, 912, 2013, 913, 2277, 914, 2403, 341, 915, 4902, 916, 342, 1594, 4440, 917, 343, 344, 4559, 918, 2439, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 371, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2345, 4471, 952, 953, 401, 2475, 403, 404, 4758, 954, 955, 2437, 4139, 1482, 3602, 4434, 3721, 1592, 1825, 1698, 1519, 1352, 4202, 4409, 1977, 1917, 405, 406, 407, 408, 956, 957, 2267, 1631, 1684, 409, 2054, 3889, 958, 410, 2117, 411, 959, 412, 960, 2443, 4712, 4619, 2285, 413, 414, 415, 961, 2352, 2417, 962, 1905, 416, 963, 417, 418, 2382, 3675, 1833, 3974, 419, 964, 420, 421, 965, 966, 967, 968, 2365, 969, 970, 422, 1897, 2056, 2309, 1272, 4754, 2207, 971, 423, 4133, 3830, 4865, 972, 424, 973, 425, 4955, 426, 974, 2109, 2380, 427, 1708, 4676, 428, 4141, 429, 430, 3749, 431, 1619, 975, 4044, 3590, 1944, 4126, 1923, 1706, 432, 976, 977, 978, 4867, 4400, 433, 434, 1606, 979, 435, 980, 2604, 2895, 981, 436, 3448, 4959, 4563, 2188, 437, 438, 2055, 1692, 982, 2388, 2243, 983, 2035, 2120, 4940, 439, 440, 441, 2474, 4900, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 2242, 453, 2286, 992, 1728, 993, 4703, 994, 995, 454, 1696, 996, 2039, 997, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 466, 2328, 467, 468, 469, 1231, 1468, 470, 471, 2496, 472, 473, 1005, 474, 2108, 4725, 1006, 476, 1273, 1007, 1744, 1488, 1659, 477, 478, 4598, 1604, 4753, 2493, 479, 1008, 1009, 1010, 1011, 1012, 1013, 1014, 481, 4313, 482, 483, 1499, 484, 1015, 4761, 2091, 2386, 2513, 1016, 4073, 1777, 2172, 485, 2205, 4592, 4545, 4720, 486, 1458, 4274, 2122, 4905, 4796, 2152, 4539, 4528, 4904, 4842, 4986, 4639, 4846, 4623, 4650, 4595, 2195, 4681, 487, 2182, 4710, 1775, 2083, 4205, 1526, 4586, 1697, 1017, 2000, 4692, 2480, 2017, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 5001, 2136, 493, 2211, 4532, 494, 2421, 495, 4090, 2378, 2210, 2247, 2478, 2047, 496, 4620, 4978, 1019, 2012, 1803, 3642, 497, 1322, 4752, 1020, 498, 1021, 2235, 2302, 2488, 4665, 4699, 4608, 4781, 499, 2049, 2404, 4685, 500, 501, 1022, 502, 1333, 503, 1023, 4686, 4804, 504, 4727, 505, 506, 4805, 2065, 1890, 1024, 507, 508, 509, 2110, 1025, 510, 1026, 3608, 1732, 511, 2397, 1864, 1816, 1531, 512, 513, 2451, 3636, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 4746, 2489, 4922, 2145, 1030, 4832, 4950, 1261, 4027, 3478, 3763, 2959, 1293, 2462, 517, 3197, 1595, 3413, 2067, 1031, 2228, 1448, 518, 4698, 4616, 2057, 2305, 2133, 2061, 4526, 4617, 1032, 1033, 2429, 2311, 1675, 1797, 519, 4578, 2271, 4693, 4604, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 1038, 536, 1039, 537, 538, 539, 1040, 3767, 1929, 1041, 1042, 3683, 2100, 4853, 2300, 2165, 540, 2105, 541, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1649, 543, 544, 545, 546, 1045, 547, 2225, 4240, 3580, 4680, 1046, 548, 549, 1047, 550, 2598, 551, 552, 553, 3819, 1259, 4062, 1048, 554, 2841, 1049, 1050, 3515, 1433, 1323, 4301, 555, 556, 1051, 557, 558, 1052, 1053, 3738, 1567, 559, 1054, 1270, 3857, 1055, 2151, 560, 2175, 2089, 4514, 2198, 2193, 1257, 1238, 3662, 561, 2186, 1057, 562, 1058, 563, 4872, 2097, 2436, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 3934, 4080, 4352, 574, 1700, 3739, 1565, 1419, 1811, 3950, 1690, 1397, 3716, 1569, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 575, 4443, 3761, 1346, 1587, 1072, 3592, 576, 1891, 4469, 1275, 577, 1950, 1963, 4129, 1073, 1296, 1360, 1551, 3733, 4111, 3993, 4108, 3949, 1287, 1713, 1556, 1736, 1995, 1435, 1424, 1705, 1861, 1197, 1254, 4120, 1545, 4417, 1469, 1292, 2185, 1785, 1236, 4293, 1827, 3839, 3983, 4395, 1957, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 2304, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2113, 590, 591, 1085, 2256, 592, 2308, 593, 594, 595, 4931, 2407, 1858, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 2321, 1086, 601, 4017, 4477, 1794, 2483, 4776, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 2320, 2069, 610, 611, 1093, 612, 1094, 3701, 4389, 613, 4897, 1603, 614, 1095, 2154, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4855, 4847, 2398, 2239, 1101, 1667, 1384, 623, 4745, 3700, 1788, 1600, 624, 2098, 625, 4550, 4721, 4960, 2473, 4792, 4675, 1102, 1423, 626, 2771, 627, 1339, 3875, 628, 629, 3070, 4565, 2118, 2015, 4818, 4860, 2557, 2795, 2906, 2103, 4869, 2029, 3299, 3118, 4932, 4838, 4901, 4908, 4996, 4779, 2377, 4657, 4646, 4875, 4627, 4515, 4876, 4965, 4605, 4576, 2028, 4985, 4799, 4850, 1103, 4757, 4747, 1614, 1947, 2237, 630, 631, 4997, 4618, 4479, 3766, 632, 1104, 1105, 4886, 1106, 2432, 4827, 633, 1107, 4530, 634, 2371, 1108, 4536, 635, 1813, 636, 2431, 4866, 4991, 4814, 4560, 4956, 637, 4555, 1109, 4633, 2213, 638, 639, 3706, 1110, 640, 1111, 641, 4581, 4893, 4831, 2481, 4644, 4734, 2190, 4881, 4837, 4888, 4648, 642, 4548, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4629, 1115, 4918, 4688, 3920, 644, 4723, 4949, 4607, 4719, 1116, 645, 646, 4611, 2353, 4603, 4762, 4819, 2221, 4636, 4992, 2307, 647, 1801, 4279, 3762, 1637, 1509, 4843, 4797, 4590, 4516, 4541, 2200, 4580, 4858, 648, 649, 2477, 4724, 4551, 4674, 1118, 1119, 2965, 2369, 2695, 3293, 650, 651, 4862, 4722, 4944, 1120, 652, 1121, 4983, 4784, 4877, 653, 654, 1122, 3264, 655, 2076, 4849, 656, 657, 2447, 658, 659, 2191, 4035, 2910, 3391, 2808, 3099, 2913, 3676, 2852, 3284, 2731, 1609, 2375, 3537, 3994, 1319, 4467, 1802, 1227, 1623, 660, 4844, 4714, 4979, 4812, 2192, 1123, 661, 2364, 662, 663, 664, 1124, 1125, 665, 2487, 4899, 2138, 1126, 666, 667, 4569, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 674, 2456, 4588, 4963, 4851, 4558, 4557, 4697, 4610, 4773, 4687, 4708, 4910, 2123, 1136, 1137, 2358, 5005, 2208, 4952, 4945, 1933, 1731, 1793, 675, 676, 3342, 1138, 1139, 677, 4840, 4597, 4870, 1140, 4990, 4954, 4916, 678, 1141, 4737, 4527, 2253, 4531, 679, 1142, 1143, 680, 4771, 1144, 4638, 681, 4705, 4525, 4898, 2325, 2415, 5004, 1145, 682, 683, 2141, 1146, 684, 685, 2445, 686, 687, 4861, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4690, 4859, 4793, 4575, 2160, 4828, 4731, 4802, 4750, 1150, 2081, 2258, 697, 1795, 1372, 1695, 1866, 1151, 4115, 1719, 2280, 4662, 698, 2176, 2217, 1920, 699, 700, 701, 4999, 1544, 4739, 2512, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 2340, 4182, 706, 4652, 2442, 2484, 707, 708, 709, 4667, 1153, 2412, 4732, 4476, 2053, 710, 1375, 711, 712, 713, 4738, 4811, 4570, 1245, 714, 2045, 4707, 715, 2169, 4713, 1841, 4234, 4924, 1154, 1314, 4096, 2023, 717, 4895, 2414, 718, 1155, 719, 720, 2063, 1311, 4921, 721, 4833, 4903, 4885, 4666, 2249, 4217, 1156, 723, 1157, 1627, 2116, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 2248, 730, 731, 1846, 2507, 2368, 1162, 2019, 2333, 3815, 2005, 2266, 1964, 2347, 732, 2177, 2059, 4958, 4927, 1822, 4815, 1262, 2094, 4480, 1537, 1291, 2419, 1318, 4628, 4294, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4835, 2259, 2275, 1166, 4968, 2423, 737, 1167, 4989, 4612, 2127, 1745, 1973, 4767, 1168, 2273, 2310, 3807, 4307, 1169, 2303, 1454, or 1781.

33. The VP capsid polypeptide of any one of claims 1-28, wherein:
(a) X³ of SEQ ID NO: 5014 is not E,
(b) X⁶ of SEQ ID NO: 5014 is not E, or
(c) X³ of SEQ ID NO: 5014 is not E and X⁶ of SEQ ID NO: 5014 is not E.

34. The VP capsid polypeptide of claim 33, wherein the 581 to 589 region of the VP capsid polypeptide has a sequence of any one of SEQ ID NOs: 2498, 4884, 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 4889, 21, 3192, 4942, 22, 23, 1638, 748, 24, 25, 749, 26, 27, 1703, 1836, 28, 2038, 2301, 3660, 29, 750, 30, 31, 32, 2095, 1640, 33, 34, 35, 36, 37, 4742, 2450, 38, 4925, 39, 751, 752, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2080, 2072, 2034, 50, 1308, 1230, 3960, 1378, 2994, 3080, 2589, 3681, 51, 4694, 2257, 52, 760, 53, 2389, 4934, 4951, 761, 4591, 762, 4659, 54, 2146, 3925, 55, 3654, 2087, 2346, 4766, 4682, 4984, 4744, 56, 763, 764, 4626, 4702, 57, 58, 59, 60, 61, 4086, 1740, 1660, 765, 766, 62, 1966, 2032, 2499, 2332, 767, 63, 2096, 2181, 4913, 64, 768, 769, 770, 2384, 2402, 771, 65, 66, 2111, 67, 772, 2458, 2197, 68, 3816, 69, 2599, 2706, 2497, 70, 2234, 2183, 2399, 71, 4769, 2366, 72, 73, 74, 3865, 1549, 1361, 2453, 3882, 773, 3713, 1942, 2140, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2132, 2379, 81, 82, 4065, 83, 84, 2030, 1294, 2413, 2064, 1484, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 93, 2356, 784, 785, 786, 94, 95, 787, 788, 1831, 1867, 96, 4458, 97, 1970, 4656, 2173, 1936, 2349, 1590, 4585, 98, 99, 100, 789, 790, 1298, 101, 1688, 791, 792, 2007, 4556, 2070, 102, 793, 4632, 103, 794, 795, 104, 105, 106, 2229, 796, 4980, 4691, 107, 2440, 1849, 108, 2014, 797, 4736, 2509, 2018, 2227, 4787, 109, 1579, 110, 1906, 111, 2428, 798, 2075, 112, 2411, 113, 2359, 799, 4848, 800, 2461, 1444, 2313, 2350, 4579, 4821, 801, 114, 2329, 4834, 115, 1344, 116, 802, 117, 118, 119, 4929, 4677, 120, 2158, 121, 803, 804, 1463, 805, 122, 2139, 2290, 1760, 2465, 123, 2424, 2058, 2230, 124, 4717, 806, 125, 126, 2370, 127, 4521, 128, 4971, 4994, 807, 2085, 4533, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 4544, 5009, 144, 145, 816, 4654, 4816, 1634, 1302, 817, 146, 818, 147, 148, 149, 4546, 150, 151, 152, 153, 154, 155, 819, 820, 156, 2149, 157, 2295, 821, 158, 159, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 2170, 4809, 4520, 180, 826, 827, 181, 828, 4695, 4911, 182, 4582, 4873, 4596, 2020, 183, 4641, 829, 184, 185, 2244, 4647, 186, 187, 188, 189, 190, 2114, 830, 191, 192, 193, 194, 2071, 4615, 2361, 195, 4852, 2187, 4914, 4947, 196, 1391, 197, 4534, 4726, 4788, 198, 199, 4920, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2241, 217, 218, 4653, 219, 1612, 220, 221, 222, 839, 223, 4953, 2360, 840, 224, 4928, 4741, 225, 2088, 4751, 5007, 4542, 226, 227, 4890, 4519, 4883, 228, 4786, 229, 230, 231, 2393, 2448, 841, 842, 232, 233, 4782, 234, 4962, 2269, 843, 235, 844, 236, 237, 4930, 845, 846, 238, 847, 4704, 239, 848, 849, 2150, 850, 851, 852, 853, 1536, 240, 854, 241, 242, 243, 244, 245, 246, 247, 248, 4923, 2245, 4696, 855, 4634, 856, 249, 250, 251, 252, 253, 254, 255, 857, 2115, 4880, 2218, 256, 257, 258, 259, 858, 859, 260, 261, 860, 861, 862, 262, 4966, 863, 864, 263, 4749, 4970, 2296, 2318, 865, 2326, 264, 1241, 265, 266, 866, 1477, 267, 867, 4977, 268, 868, 269, 4668, 5002, 270, 869, 870, 271, 871, 872, 272, 4822, 4709, 873, 4655, 4972, 2392, 874, 1252, 273, 1591, 4975, 875, 274, 2025, 275, 1980, 276, 876, 877, 2066, 277, 278, 279, 4670, 280, 2323, 2288, 2334, 4939, 4689, 281, 878, 1892, 879, 1539, 2214, 2215, 880, 2327, 881, 882, 4601, 883, 1295, 3601, 3877, 1930, 1420, 1678, 1460, 2306, 884, 282, 1799, 283, 284, 4988, 4553, 4772, 4817, 2260, 285, 1626, 2082, 885, 286, 1374, 886, 2119, 2468, 287, 2408, 1620, 1290, 2425, 2482, 4564, 4671, 2282, 288, 2354, 1354, 2033, 289, 887, 290, 888, 291, 1956, 2362, 2467, 889, 2357, 4547, 4765, 292, 4794, 2391, 4324, 293, 890, 294, 295, 4138, 891, 2297, 2254, 296, 2224, 3903, 297, 892, 3829, 298, 299, 3809, 4271, 3844, 1436, 300, 1515, 1617, 4701, 2174, 301, 1961, 4649, 5010, 302, 303, 304, 305, 2452, 2147, 306, 2293, 1589, 893, 4370, 4841, 2342, 307, 308, 309, 894, 2246, 2155, 3646, 4059, 1431, 1331, 310, 311, 4836, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 904, 1635, 4820, 905, 2506, 331, 4706, 2092, 4484, 1756, 906, 907, 4661, 2179, 4584, 4609, 332, 4907, 4635, 4529, 4683, 4523, 4561, 333, 2337, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4783, 4319, 3906, 1533, 1755, 1888, 4248, 1557, 2153, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 2444, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 2363, 908, 1935, 4711, 4728, 2460, 4517, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 910, 2979, 2542, 2338, 2492, 1762, 4472, 1472, 1529, 2319, 2131, 4057, 4937, 2048, 1976, 4507, 1583, 338, 2078, 2372, 339, 911, 1711, 340, 912, 2013, 913, 2277, 914, 2403, 341, 915, 4672, 4902, 916, 342, 1594, 4440, 917, 343, 344, 4559, 918, 2233, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2037, 352, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 2471, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 371, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2510, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 2476, 3324, 2769, 2678, 1561, 3296, 375, 2343, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 1941, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 387, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 2383, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2129, 2345, 4471, 952, 953, 402, 2475, 403, 404, 4758, 955, 2437, 4139, 1482, 3602, 4434, 3721, 1592, 1825, 1698, 1519, 1352, 4202, 4409, 1977, 1917, 405, 406, 407, 408, 956, 2272, 957, 2267, 1631, 1684, 409, 2054, 3889, 958, 410, 2117, 411, 959, 412, 960, 2443, 4712, 4619, 2285, 414, 415, 961, 2352, 2417, 962, 1905, 416, 963, 417, 418, 3675, 1833, 3974, 419, 964, 420, 421, 965, 966, 967, 968, 2365, 969, 970, 422, 1897, 2056, 2309, 4733, 1272, 4754, 2207, 971, 423, 4133, 3830, 4865, 972, 424, 973, 425, 2068, 4955, 426, 974, 427, 1708, 4676, 428, 4141, 430, 3749, 431, 1619, 975, 4044, 3590, 1944, 4126, 1923, 1706, 432, 976, 977, 978, 4867, 4400, 433, 434, 1606, 2143, 979, 435, 980, 2604, 2895, 981, 436, 3448, 4959, 4563, 2188, 437, 438, 1692, 982, 2388, 2433, 2243, 983, 2035, 2120, 4940, 439, 440, 441, 2474, 442, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 2242, 453, 2286, 992, 1728, 993, 4703, 994, 995, 454, 1696, 996, 2039, 455, 997, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 467, 468, 469, 1231, 1468, 470, 471, 2496, 472, 473, 1005, 474, 475, 2108, 4725, 1006, 476, 1273, 1007, 1744, 1488, 1659, 477, 478, 4598, 1604, 4753, 2493, 479, 1008, 1009, 480, 1010, 1011, 1012, 1013, 1014, 481, 4313, 482, 483, 1499, 484, 1015, 4761, 2091, 2513, 1016, 4073, 1777, 2172, 485, 2205, 4592, 4545, 4720, 486, 1458, 4274, 2122, 4905, 5003, 4599, 4796, 2152, 4539, 4528, 4904, 4842, 4986, 4639, 4846, 4623, 4650, 4595, 2195, 4681, 2142, 487, 2182, 4710, 1775, 2083, 4205, 1526, 4586, 4602, 4518, 1697, 1017, 2000, 4692, 4795, 2480, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 2202, 5001, 2136, 493, 2211, 4532, 494, 2421, 495, 4090, 2378, 2104, 2210, 2247, 2478, 2047, 496, 4620, 4978, 4614, 1019, 2012, 1803, 3642, 497, 1322, 1020, 498, 1021, 2235, 2302, 2488, 4665, 4540, 5006, 499, 2049, 2404, 4685, 500, 501, 1022, 502, 1333, 503, 1023, 4686, 4804, 504, 4727, 505, 506, 4938, 4805, 2065, 1890, 4891, 1024, 507, 508, 509, 1025, 510, 1026, 3608, 1732, 511, 2397, 1864, 1816, 1531, 512, 513, 2451, 3636, 5012, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 2093, 4746, 2489, 4922, 2145, 1030, 4832, 4950, 1261, 4027, 3478, 3763, 2959, 1293, 2462, 517, 3197, 1595, 3413, 2238, 1031, 2228, 1448, 518, 2057, 2305, 2133, 2061, 4526, 4617, 1033, 2429, 2311, 1675, 4785, 1797, 519, 4578, 2271, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 535, 1038, 536, 1039, 537, 538, 539, 1040, 3767, 1929, 1041, 1042, 3683, 2100, 4853, 2300, 2165, 540, 541, 1043, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 2121, 1044, 3720, 4351, 4414, 1673, 3453, 3254, 2501, 1649, 543, 544, 545, 546, 1045, 547, 2084, 4240, 3580, 4680, 1046, 548, 2062, 1047, 550, 2598, 551, 552, 553, 3819, 1259, 4062, 1048, 554, 2841, 1049, 1050, 3515, 2219, 1433, 1323, 4301, 555, 556, 1051, 557, 558, 1052, 1053, 3738, 1567, 559, 1054, 1270, 3857, 1055, 2151, 560, 2175, 2089, 1056, 2198, 2193, 1257, 1238, 3662, 561, 2186, 1057, 4637, 562, 1058, 563, 4872, 2097, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 2304, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2255, 2113, 590, 591, 1085, 2256, 592, 2308, 593, 594, 2274, 595, 4931, 2407, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 2321, 1086, 601, 4017, 4477, 1794, 2483, 4776, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 610, 611, 1093, 612, 1094, 3701, 4389, 613, 4897, 1603, 614, 1095, 2154, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4801, 2398, 2239, 1101, 1667, 1384, 623, 4745, 3700, 1788, 1600, 624, 2098, 625, 4550, 4721, 4960, 4878, 2473, 4792, 4675, 1102, 1423, 626, 2771, 627, 1339, 3875, 628, 629, 3070, 4565, 2118, 4818, 4860, 2557, 2795, 2906, 2103, 4869, 2029, 3299, 3118, 4932, 4838, 4901, 2377, 4657, 4646, 4875, 4627, 4515, 4876, 4965, 4605, 4576, 2028, 4985, 4799, 4850, 1103, 4747, 1614, 1947, 2237, 630, 631, 4618, 4479, 3766, 632, 1104, 1105, 4886, 1106, 2432, 4827, 633, 1107, 4530, 634, 2371, 4651, 1108, 4536, 635, 1813, 636, 2431, 4866, 4991, 4814, 4560, 4956, 637, 4555, 1109, 4941, 4577, 2213, 638, 639, 3706, 1110, 640, 1111, 641, 4581, 4893, 4831, 2481, 4644, 4734, 2190, 4524, 4881, 4837, 4888, 4648, 642, 4548, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4760, 1115, 4918, 4688, 3920, 644, 4723, 4949, 4748, 4607, 4719, 1116, 645, 646, 4611, 2353, 4645, 4998, 2307, 647, 1801, 4279, 3762, 1637, 1509, 4843, 4797, 4590, 4516, 4995, 4541, 2200, 4580, 4858, 648, 649, 2502, 1117, 4780, 4800, 4551, 4674, 1118, 1119, 2965, 2369, 2695, 3293, 650, 651, 4730, 4862, 4722, 4944, 1120, 652, 1121, 4983, 4784, 4877, 4643, 653, 654, 4909, 1122, 3264, 655, 2076, 4849, 656, 657, 2447, 658, 659, 2191, 4035, 2910, 3391, 2808, 3099, 2913, 3676, 2852, 3284, 2731, 1609, 2375, 2201, 3537, 3994, 1319, 4467, 1802, 1227, 1623, 660, 4844, 2086, 4770, 2192, 1123, 661, 2364, 662, 663, 664, 1124, 1125, 665, 2487, 4899, 2138, 1126, 666, 667, 4569, 4554, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 674, 2456, 4606, 4910, 2123, 1136, 1137, 2358, 5005, 2208, 4952, 4945, 1933, 1731, 1793, 675, 676, 3342, 1138, 1139, 677, 4840, 4597, 4870, 1140, 4990, 4954, 4916, 678, 1141, 4737, 4527, 2253, 4531, 679, 1142, 1143, 680, 4771, 1144, 4638, 681, 4705, 4525, 4522, 4898, 2325, 2415, 5004, 1145, 682, 683, 1146, 684, 685, 2445, 686, 687, 4861, 2157, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4793, 4575, 2160, 4828, 4731, 4718, 4802, 4750, 1150, 2081, 2258, 697, 1795, 1372, 1695, 1866, 1151, 4115, 1719, 2280, 4662, 698, 2176, 2194, 2217, 1920, 699, 700, 701, 4999, 1544, 4739, 2512, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 4824, 705, 2340, 4182, 706, 4652, 2442, 2484, 707, 708, 709, 4667, 1153, 2412, 4476, 2053, 710, 1375, 711, 712, 713, 4829, 4738, 4811, 4570, 4961, 1245, 714, 2045, 4707, 715, 2169, 4713, 4879, 1841, 4234, 4924, 1154, 1314, 4096, 2023, 716, 717, 4895, 2414, 718, 1155, 719, 720, 2063, 1311, 4921, 721, 4642, 722, 4885, 4666, 2249, 4217, 1156, 723, 1157, 1627, 2116, 4566, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 730, 731, 2024, 1846, 2507, 2368, 2019, 2333, 3815, 2005, 2266, 1964, 2347, 2385, 732, 2177, 2059, 4958, 1822, 4815, 1262, 2094, 4480, 1537, 1291, 2419, 1318, 2283, 4976, 4294, 2486, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4679, 4835, 2259, 4839, 4571, 2275, 1166, 4968, 2250, 4912, 2423, 737, 1167, 4989, 4684, 4669, 4552, 4612, 2127, 1745, 1973, 4767, 1168, 2273, 2310, 3807, 4307, 1169, 2303, 1454, or 1781.

35. The VP capsid polypeptide of claim 33, wherein the 581 to 589 region of the VP capsid polypeptide has a sequence of any one of SEQ ID NOs: 2498, 4884, 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 2469, 4889, 21, 3192, 4942, 22, 23, 1638, 748, 24, 25, 749, 26, 27, 1703, 1836, 28, 2038, 2301, 3660, 29, 750, 30, 31, 32, 2095, 1640, 33, 35, 36, 37, 4742, 2450, 38, 4925, 39, 751, 752, 2144, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2080, 50, 1308, 1230, 3960, 1378, 2994, 3080, 2589, 3681, 51, 4694, 2257, 2167, 52, 760, 53, 2389, 4934, 4951, 4755, 761, 762, 4659, 54, 2146, 3925, 55, 3654, 2087, 2346, 4766, 4682, 4984, 4744, 56, 763, 764, 4626, 4702, 58, 59, 60, 61, 4086, 1740, 1660, 765, 766, 62, 1966, 2032, 2499, 2332, 767, 63, 2096, 2181, 4913, 2438, 2041, 64, 768, 769, 770, 2384, 2402, 771, 65, 66, 2111, 67, 772, 2458, 2197, 68, 3816, 69, 2599, 2706, 2497, 70, 2234, 2183, 2399, 71, 4769, 2366, 72, 73, 74, 3865, 1549, 1361, 2453, 3882, 773, 3713, 1942, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2132, 2379, 82, 4065, 83, 84, 2030, 1294, 2413, 2064, 1484, 2299, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 93, 2356, 784, 785, 786, 94, 95, 787, 788, 1831, 1867, 96, 4458, 97, 1970, 4656, 2173, 1936, 2349, 1590, 4585, 98, 99, 100, 789, 790, 1298, 101, 1688, 791, 792, 2007, 2070, 102, 793, 4632, 103, 794, 795, 104, 105, 106, 2229, 796, 4980, 4691, 107, 2440, 1849, 108, 2014, 797, 4736, 2509, 2018, 2227, 4787, 109, 1579, 110, 1906, 111, 2428, 798, 112, 2411, 113, 2359, 799, 4848, 800, 2461, 1444, 2313, 2350, 4579, 4821, 801, 114, 2329, 4864, 4834, 115, 1344, 116, 802, 117, 118, 119, 4929, 4677, 120, 4791, 2158, 121, 803, 804, 1463, 805, 122, 2139, 2290, 1760, 2465, 2424, 2058, 2230, 124, 4717, 806, 125, 126, 2370, 127, 4521, 128, 4971, 4994, 807, 2085, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 4544, 144, 145, 816, 4654, 2125, 4663, 4810, 4816, 1634, 1302, 817, 146, 818, 147, 148, 149, 4546, 150, 151, 152, 153, 154, 155, 819, 820, 156, 2149, 157, 2295, 821, 158, 159, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 2170, 4809, 4520, 180, 826, 827, 181, 828, 4695, 2130, 4911, 182, 4582, 4873, 4596, 2020, 2189, 183, 4641, 829, 184, 185, 2244, 4647, 186, 187, 188, 189, 190, 2114, 830, 191, 192, 193, 194, 2071, 4615, 2361, 195, 4852, 2187, 4914, 4947, 196, 1391, 197, 4534, 4726, 4788, 198, 199, 4920, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2090, 2241, 217, 218, 4653, 219, 1612, 220, 221, 222, 839, 223, 4953, 4974, 4790, 4915, 2464, 4567, 4774, 4964, 2360, 840, 224, 4928, 4741, 225, 2088, 4751, 5007, 4542, 226, 227, 4890, 4519, 4883, 228, 4786, 229, 230, 231, 2393, 2448, 841, 842, 232, 233, 4782, 234, 4962, 2269, 843, 235, 844, 236, 237, 4930, 845, 846, 238, 847, 4704, 239, 848, 849, 2150, 850, 851, 852, 853, 1536, 240, 854, 241, 242, 243, 244, 245, 246, 247, 248, 4923, 2245, 4696, 855, 4634, 856, 249, 250, 251, 252, 253, 254, 255, 857, 2115, 4880, 256, 257, 258, 259, 858, 859, 260, 261, 860, 861, 862, 262, 4966, 863, 864, 263, 4749, 4970, 2296, 2318, 4535, 865, 2326, 264, 1241, 265, 266, 866, 1477, 267, 867, 4977, 268, 868, 269, 4668, 5002, 270, 869, 870, 271, 871, 872, 272, 4822, 4709, 873, 4655, 4972, 2392, 2317, 874, 1252, 273, 1591, 4975, 875, 274, 2025, 275, 1980, 276, 876, 877, 2066, 277, 278, 279, 4670, 280, 2323, 2288, 2334, 4689, 281, 878, 2416, 1892, 879, 1539, 2214, 2215, 880, 2327, 881, 882, 4601, 883, 1295, 3601, 3877, 1930, 1420, 1678, 1460, 2306, 884, 282, 1799, 283, 284, 4988, 4871, 4553, 4772, 4817, 2260, 285, 1626, 2082, 885, 286, 1374, 886, 2119, 2504, 2468, 287, 1620, 1290, 2425, 2482, 4671, 2282, 288, 2354, 1354, 2033, 289, 887, 290, 888, 291, 1956, 2467, 889, 2357, 4547, 4765, 292, 4794, 2391, 4324, 890, 294, 295, 4138, 891, 2297, 296, 2224, 3903, 297, 892, 3829, 298, 299, 3809, 4271, 3844, 1436, 300, 1515, 1617, 4701, 2174, 301, 1961, 4649, 5010, 302, 2060, 303, 304, 305, 2452, 2147, 4574, 4538, 306, 2293, 1589, 893, 4946, 4370, 4841, 2342, 307, 308, 309, 894, 2246, 2155, 3646, 4059, 1431, 1331, 310, 311, 4836, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 904, 1635, 4820, 905, 2506, 331, 4706, 2092, 4484, 1756, 906, 907, 4661, 2179, 4584, 332, 4907, 4635, 4529, 4683, 4523, 4561, 333, 2337, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4783, 4319, 3906, 1533, 1755, 1888, 4248, 1557, 2153, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 2444, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 908, 1935, 4711, 4728, 2460, 4517, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 4943, 3002, 2986, 910, 2979, 2542, 2331, 2492, 1762, 4472, 1472, 1529, 2319, 2131, 4057, 2048, 4887, 1976, 4507, 1583, 338, 2078, 2372, 2102, 339, 911, 1711, 340, 912, 2013, 913, 2277, 914, 341, 915, 4672, 4902, 916, 342, 1594, 4440, 917, 343, 344, 4559, 918, 2233, 2439, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 2471, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 375, 2343, 2348, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 1387, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 938, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 2383, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2129, 2345, 4471, 952, 953, 401, 402, 2475, 403, 404, 4758, 955, 2437, 4139, 1482, 3602, 4434, 3721, 1592, 1825, 1698, 1519, 1352, 4202, 4409, 1977, 1917, 405, 406, 407, 408, 956, 2272, 957, 2267, 1631, 1684, 409, 2054, 3889, 958, 410, 2117, 411, 959, 412, 960, 2443, 4712, 4619, 2285, 413, 414, 415, 961, 2352, 2417, 962, 1905, 416, 963, 417, 418, 2382, 3675, 1833, 3974, 419, 964, 421, 965, 966, 967, 4919, 968, 2365, 969, 970, 422, 1897, 2056, 2309, 4733, 1272, 4754, 2207, 971, 423, 4133, 3830, 4865, 972, 424, 973, 425, 2068, 4955, 426, 974, 2109, 2380, 427, 1708, 4676, 428, 4141, 429, 430, 3749, 431, 1619, 975, 4044, 3590, 1944, 4126, 1923, 1706, 432, 976, 977, 978, 4867, 4400, 433, 434, 1606, 2143, 979, 435, 980, 2604, 2895, 981, 436, 3448, 4959, 2188, 437, 438, 2055, 1692, 982, 2388, 2433, 2243, 983, 2035, 2120, 4940, 439, 440, 2474, 442, 4900, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 453, 992, 1728, 993, 4703, 994, 995, 454, 1696, 996, 2039, 455, 997, 4874, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 466, 2328, 467, 468, 469, 1231, 1468, 470, 471, 2496, 472, 473, 1005, 474, 475, 2108, 4725, 476, 1273, 1007, 1744, 1488, 1659, 477, 478, 4598, 1604, 4753, 2493, 479, 1008, 1009, 480, 1011, 1012, 1013, 1014, 481, 4313, 482, 483, 1499, 484, 1015, 4761, 2091, 2386, 2513, 1016, 4073, 1777, 2172, 485, 2205, 4592, 4545, 486, 1458, 4274, 2122, 4905, 5003, 4599, 4796, 2152, 4539, 4528, 4904, 4842, 4986, 4846, 4650, 4595, 2195, 4681, 2142, 487, 4710, 1775, 4205, 1526, 4586, 4602, 4518, 1697, 1017, 2000, 4692, 4795, 2480, 2017, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 2202, 5001, 2136, 493, 2211, 4532, 494, 2421, 495, 4090, 2378, 2104, 2210, 2247, 2478, 2047, 496, 4620, 4978, 4614, 1019, 2012, 1803, 3642, 497, 1322, 4752, 1020, 498, 1021, 2235, 2302, 2488, 4665, 4540, 5006, 4699, 4608, 4781, 499, 2049, 4685, 500, 501, 1022, 502, 1333, 503, 1023, 4686, 4804, 504, 4727, 505, 506, 4938, 4805, 2065, 1890, 4891, 1024, 507, 508, 509, 2110, 1025, 510, 1026, 3608, 1732, 511, 2397, 1864, 1816, 1531, 512, 513, 2451, 3636, 5012, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 2093, 4746, 2489, 4922, 2145, 1030, 4832, 4950, 1261, 4027, 3478, 3763, 2959, 1293, 2462, 517, 3197, 1595, 3413, 2238, 2067, 1031, 2228, 518, 4698, 4616, 2057, 2305, 2133, 2061, 4526, 4617, 1032, 1033, 2429, 2311, 1675, 4785, 1797, 519, 4578, 2271, 4693, 4604, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 535, 1038, 536, 1039, 537, 538, 539, 1040, 3767, 1929, 1042, 3683, 2100, 4853, 2300, 2165, 540, 2105, 541, 1043, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 1044, 3720, 4351, 4414, 1673, 3453, 3254, 2501, 1649, 543, 544, 545, 546, 1045, 547, 4240, 3580, 4680, 1046, 548, 2062, 549, 1047, 550, 2598, 551, 552, 553, 3819, 1259, 4062, 1048, 554, 2841, 1049, 1050, 3515, 2219, 1433, 1323, 4301, 555, 556, 1051, 557, 558, 1052, 1053, 3738, 1567, 559, 1054, 1270, 3857, 1055, 2151, 560, 2175, 2089, 1056, 4514, 2198, 2193, 1257, 1238, 3662, 561, 2186, 1057, 4637, 562, 1058, 563, 4872, 2097, 2436, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1071, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2185, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2255, 2113, 590, 591, 1085, 592, 2308, 593, 594, 2274, 595, 4931, 2407, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 1086, 601, 4017, 4477, 1794, 2483, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 2320, 2069, 610, 611, 1093, 612, 1094, 3701, 4389, 613, 4897, 1603, 614, 1095, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4801, 4855, 4847, 2398, 2239, 1101, 1667, 1384, 623, 4745, 3700, 1788, 1600, 624, 2098, 625, 4550, 4721, 4960, 4878, 2473, 4792, 4675, 1102, 1423, 626, 2771, 627, 1339, 3875, 628, 629, 3070, 4565, 2118, 2015, 4818, 2557, 2795, 2906, 2103, 4869, 2029, 3299, 3118, 4932, 4838, 4901, 4908, 4996, 4779, 4657, 4646, 4875, 4627, 4515, 4876, 4965, 4605, 4576, 2028, 4985, 4850, 1103, 4757, 4747, 1614, 1947, 2237, 630, 631, 4997, 4618, 4479, 3766, 632, 1104, 1105, 4886, 1106, 2432, 633, 1107, 4530, 634, 2371, 4651, 1108, 4536, 635, 1813, 636, 2431, 4866, 4991, 4814, 4560, 4956, 637, 4555, 1109, 4941, 4577, 4633, 2213, 638, 639, 3706, 1110, 640, 1111, 641, 4581, 4893, 4831, 2481, 4644, 4734, 2190, 4524, 4881, 4837, 4888, 4648, 642, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4760, 4629, 1115, 4918, 4688, 3920, 644, 4723, 4949, 4748, 4607, 4719, 1116, 645, 646, 4611, 2353, 4645, 4603, 4762, 4819, 2221, 4636, 4992, 4998, 2307, 647, 1801, 4279, 3762, 1637, 1509, 4843, 4797, 4590, 4995, 4541, 2200, 4580, 4858, 648, 649, 2502, 1117, 4780, 4800, 2477, 4724, 4551, 4674, 1118, 1119, 2965, 2369, 2695, 3293, 650, 651, 4730, 4862, 4722, 4944, 1120, 652, 1121, 4983, 4784, 4877, 4643, 653, 654, 4909, 1122, 3264, 655, 2076, 4849, 656, 657, 2447, 658, 659, 4035, 2910, 3391, 2808, 3099, 2913, 3676, 2852, 3284, 2731, 1609, 2375, 2201, 3537, 3994, 1319, 4467, 1802, 1227, 1623, 660, 4844, 2086, 4770, 4714, 4979, 4812, 2192, 1123, 661, 2364, 662, 663, 664, 1124, 1125, 665, 2487, 4899, 2138, 1126, 666, 667, 4569, 4554, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 2456, 4606, 4588, 4963, 4851, 4558, 4557, 4697, 4610, 4773, 4687, 4708, 4910, 2123, 1136, 1137, 2358, 5005, 2208, 4952, 4945, 1933, 1731, 1793, 675, 676, 3342, 1138, 677, 4597, 4870, 1140, 4990, 4954, 4916, 678, 1141, 4737, 4527, 2253, 4531, 679, 1142, 1143, 680, 4771, 1144, 4638, 681, 4705, 4525, 4522, 4898, 2325, 2415, 5004, 1145, 682, 683, 2141, 1146, 684, 685, 2445, 686, 687, 4861, 2157, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4690, 4859, 4793, 4575, 2160, 4828, 4731, 4718, 4802, 4750, 1150, 2081, 2258, 697, 1795, 1372, 1695, 1866, 1151, 4115, 1719, 2280, 4662, 698, 2176, 2194, 2217, 1920, 699, 700, 701, 4999, 1544, 4739, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 4824, 705, 2340, 4182, 706, 2442, 2484, 707, 708, 709, 4667, 1153, 2412, 4732, 4476, 2053, 710, 1375, 711, 712, 713, 4829, 4738, 4811, 4570, 4961, 1245, 714, 2045, 4707, 715, 2169, 4713, 1841, 4234, 4924, 1154, 1314, 4096, 2023, 716, 717, 4895, 718, 1155, 719, 720, 2063, 1311, 721, 4642, 722, 4833, 4903, 4885, 4666, 2249, 4217, 1156, 723, 1157, 1627, 2116, 4566, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 2248, 730, 731, 2024, 1846, 2507, 2368, 1162, 2019, 2333, 3815, 2005, 2266, 1964, 2347, 2385, 732, 2177, 2059, 4958, 4927, 1822, 4815, 1262, 2094, 4480, 1537, 1291, 2419, 1318, 2283, 4976, 4628, 4294, 2486, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4679, 4835, 2259, 4839, 4571, 1166, 4968, 2250, 4912, 2423, 737, 1167, 4989, 4684, 4669, 4552, 4612, 2127, 1745, 1973, 4767, 1168, 2273, 2310, 3807, 4307, 1169, 2303, 1454, or 1781.

36. The VP capsid polypeptide of claim 33, wherein the 581 to 589 region of the VP capsid polypeptide has a sequence of any one of SEQ ID NOs: 2498, 4884, 738, 739, 4177, 1289, 740, 14, 15, 741, 742, 743, 16, 744, 17, 18, 745, 3477, 1548, 3051, 2982, 2171, 2877, 746, 19, 20, 3036, 3426, 3073, 3169, 747, 2406, 3362, 2784, 4889, 21, 3192, 4942, 22, 23, 1638, 748, 24, 25, 749, 26, 27, 1703, 1836, 28, 2038, 2301, 3660, 29, 750, 30, 31, 32, 2095, 1640, 33, 35, 36, 37, 4742, 2450, 38, 4925, 39, 751, 752, 1653, 1205, 3795, 753, 4288, 4219, 2770, 2962, 4241, 4107, 2718, 3317, 4391, 4053, 3263, 2553, 3947, 2710, 2608, 3467, 2796, 2961, 2529, 3320, 2674, 2635, 3446, 3053, 3250, 3688, 1182, 1998, 4320, 3351, 2655, 3514, 2779, 3194, 3319, 3224, 3281, 3708, 2807, 3562, 3274, 3374, 3013, 3416, 2981, 3065, 2777, 3469, 2977, 2638, 2972, 3059, 2574, 3380, 4262, 3167, 3368, 2781, 3547, 3386, 1225, 2960, 3485, 3370, 3443, 2824, 754, 1467, 3327, 3178, 3006, 3252, 2991, 3470, 2888, 2572, 3418, 2642, 2519, 3363, 40, 2633, 41, 42, 43, 755, 2511, 44, 45, 756, 46, 757, 47, 48, 1952, 758, 2341, 2410, 49, 3122, 2551, 3718, 4806, 2216, 3484, 3577, 759, 2080, 50, 1308, 1230, 3960, 1378, 2994, 3080, 2589, 3681, 51, 4694, 2257, 52, 760, 53, 2389, 4934, 4951, 761, 762, 4659, 54, 2146, 3925, 55, 3654, 2087, 2346, 4766, 4682, 4984, 4744, 56, 763, 764, 4626, 4702, 58, 59, 60, 61, 4086, 1740, 1660, 765, 766, 62, 1966, 2032, 2499, 2332, 767, 63, 2096, 2181, 4913, 64, 768, 769, 770, 2384, 2402, 771, 65, 66, 2111, 67, 772, 2458, 2197, 68, 3816, 69, 2599, 2706, 2497, 70, 2234, 2183, 2399, 71, 4769, 2366, 72, 73, 74, 3865, 1549, 1361, 2453, 3882, 773, 3713, 1942, 75, 1312, 4501, 76, 77, 774, 1413, 775, 776, 78, 1560, 4289, 3757, 79, 3593, 4043, 1806, 3908, 4397, 1615, 4415, 3723, 2006, 3754, 1470, 4360, 80, 3945, 2854, 3523, 3669, 1356, 777, 2132, 2379, 82, 4065, 83, 84, 2030, 1294, 2413, 2064, 1484, 85, 4594, 86, 778, 87, 3891, 4456, 4040, 4496, 1982, 2001, 1478, 1189, 88, 1490, 1885, 1926, 89, 90, 91, 779, 2270, 780, 2161, 1180, 92, 781, 782, 783, 93, 2356, 784, 785, 786, 94, 95, 787, 788, 1831, 1867, 96, 4458, 97, 1970, 4656, 2173, 1936, 2349, 1590, 4585, 98, 99, 100, 789, 790, 1298, 101, 1688, 791, 792, 2007, 2070, 102, 793, 4632, 103, 794, 795, 104, 105, 106, 2229, 796, 4980, 4691, 107, 2440, 1849, 108, 2014, 797, 4736, 2509, 2018, 2227, 4787, 109, 1579, 110, 1906, 111, 2428, 798, 112, 2411, 113, 2359, 799, 4848, 800, 2461, 1444, 2313, 2350, 4579, 4821, 801, 114, 2329, 4834, 115, 1344, 116, 802, 117, 118, 119, 4929, 4677, 120, 2158, 121, 803, 804, 1463, 805, 122, 2139, 2290, 1760, 2465, 2424, 2058, 2230, 124, 4717, 806, 125, 126, 2370, 127, 4521, 128, 4971, 4994, 807, 2085, 129, 130, 131, 132, 4933, 808, 2336, 2026, 2204, 809, 810, 2281, 133, 1978, 134, 811, 812, 135, 136, 137, 138, 813, 814, 139, 4673, 140, 815, 1326, 141, 142, 143, 2344, 4544, 144, 145, 816, 4654, 4816, 1634, 1302, 817, 146, 818, 147, 148, 149, 4546, 150, 151, 152, 153, 154, 155, 819, 820, 156, 2149, 157, 2295, 821, 158, 159, 160, 161, 4631, 2441, 162, 163, 164, 165, 166, 822, 167, 168, 169, 170, 171, 823, 172, 4613, 824, 1651, 173, 1483, 174, 175, 825, 176, 177, 178, 179, 4624, 2170, 4809, 4520, 180, 826, 827, 181, 828, 4695, 4911, 182, 4582, 4873, 4596, 2020, 183, 4641, 829, 184, 185, 2244, 4647, 186, 187, 188, 189, 190, 2114, 830, 191, 192, 193, 194, 2071, 4615, 2361, 195, 4852, 2187, 4914, 4947, 196, 1391, 197, 4534, 4726, 4788, 198, 199, 4920, 831, 2240, 2223, 200, 201, 202, 203, 2106, 2503, 4775, 204, 205, 832, 833, 206, 834, 2232, 2294, 207, 2166, 208, 209, 210, 835, 836, 1334, 837, 3901, 3312, 2649, 1301, 211, 212, 1450, 4386, 3028, 213, 1446, 214, 2206, 215, 1896, 1522, 3490, 838, 216, 2241, 217, 218, 4653, 219, 1612, 220, 221, 222, 839, 223, 4953, 2360, 840, 224, 4928, 4741, 225, 2088, 4751, 5007, 4542, 226, 227, 4890, 4519, 4883, 228, 4786, 229, 230, 231, 2393, 2448, 841, 842, 232, 233, 4782, 234, 4962, 2269, 843, 235, 844, 236, 237, 4930, 845, 846, 238, 847, 4704, 239, 848, 849, 2150, 850, 851, 852, 853, 1536, 240, 854, 241, 242, 243, 244, 245, 246, 247, 248, 4923, 2245, 4696, 855, 4634, 856, 249, 250, 251, 252, 253, 254, 255, 857, 2115, 4880, 256, 257, 258, 259, 858, 859, 260, 261, 860, 861, 862, 262, 4966, 863, 864, 263, 4749, 4970, 2296, 2318, 865, 2326, 264, 1241, 265, 266, 866, 1477, 267, 867, 4977, 268, 868, 269, 4668, 5002, 270, 869, 870, 271, 871, 872, 272, 4822, 4709, 873, 4655, 4972, 2392, 874, 1252, 273, 1591, 4975, 875, 274, 2025, 275, 1980, 276, 876, 877, 2066, 277, 278, 279, 4670, 280, 2323, 2288, 2334, 4689, 281, 878, 1892, 879, 1539, 2214, 2215, 880, 2327, 881, 882, 4601, 883, 1295, 3601, 3877, 1930, 1420, 1678, 1460, 2306, 884, 282, 1799, 283, 284, 4988, 4553, 4772, 4817, 2260, 285, 1626, 2082, 885, 286, 1374, 886, 2119, 2468, 287, 1620, 1290, 2425, 2482, 4671, 2282, 288, 2354, 1354, 2033, 289, 887, 290, 888, 291, 1956, 2467, 889, 2357, 4547, 4765, 292, 4794, 2391, 4324, 890, 294, 295, 4138, 891, 2297, 296, 2224, 3903, 297, 892, 3829, 298, 299, 3809, 4271, 3844, 1436, 300, 1515, 1617, 4701, 2174, 301, 1961, 4649, 5010, 302, 303, 304, 305, 2452, 2147, 306, 2293, 1589, 893, 4370, 4841, 2342, 307, 308, 309, 894, 2246, 2155, 3646, 4059, 1431, 1331, 310, 311, 4836, 4948, 312, 895, 1779, 313, 314, 315, 316, 896, 1558, 1552, 317, 2312, 318, 319, 897, 320, 898, 321, 322, 899, 1958, 2022, 2016, 900, 2454, 323, 2427, 2134, 324, 901, 325, 902, 326, 2373, 4803, 2459, 1491, 327, 328, 903, 2463, 2264, 329, 330, 904, 1635, 4820, 905, 2506, 331, 4706, 2092, 4484, 1756, 906, 907, 4661, 2179, 4584, 332, 4907, 4635, 4529, 4683, 4523, 4561, 333, 2337, 3536, 2701, 2799, 3332, 3294, 3159, 3069, 2637, 3025, 2782, 3247, 3350, 2692, 2586, 3004, 3049, 2694, 2650, 3554, 2983, 3403, 3214, 3163, 2541, 3090, 3097, 3462, 1430, 2941, 3486, 2842, 3558, 3104, 3037, 3419, 2780, 2931, 1276, 2928, 2609, 3068, 3128, 2548, 2538, 2618, 2789, 3228, 2657, 3189, 2128, 2863, 3307, 3300, 2546, 3048, 3543, 2946, 2975, 3346, 3067, 2728, 2955, 3408, 3027, 3094, 3203, 2988, 2751, 2953, 2566, 2578, 2823, 3063, 3335, 3000, 3181, 3011, 3138, 3389, 2805, 3074, 2894, 3149, 2560, 3417, 3265, 3378, 2901, 2837, 2822, 2745, 3473, 3530, 3040, 2597, 3509, 3522, 3161, 2926, 3209, 3105, 3400, 2630, 2905, 2671, 2818, 2806, 2702, 2709, 2400, 3549, 3538, 2534, 2773, 2623, 3491, 3243, 3136, 2693, 3164, 3343, 2918, 2555, 2794, 2679, 3510, 3110, 2672, 3442, 2929, 3184, 3488, 2135, 4763, 3414, 3093, 2996, 3141, 3544, 3102, 2856, 2957, 3466, 3461, 2788, 2660, 3344, 3204, 2720, 3379, 2826, 1789, 2396, 2785, 3249, 3527, 4460, 3352, 2051, 2569, 4457, 2011, 4857, 2163, 4783, 4319, 3906, 1533, 1755, 1888, 4248, 1557, 2153, 3804, 1363, 1934, 1602, 1404, 4188, 1330, 4349, 3337, 3533, 2629, 3038, 2539, 3567, 3291, 3348, 3115, 3359, 2993, 3079, 3506, 2527, 2963, 2938, 2516, 3113, 1345, 2639, 3143, 3044, 2747, 3154, 3280, 3039, 2743, 2902, 3056, 2554, 2522, 3103, 3210, 3434, 2656, 4251, 4166, 4196, 3206, 3328, 2588, 3553, 2714, 3078, 3222, 3137, 3357, 2717, 2944, 3012, 3220, 3202, 2811, 3083, 3269, 3566, 2699, 2515, 3023, 2533, 3288, 3075, 3005, 2685, 3058, 2976, 2622, 3333, 3463, 2939, 3521, 3268, 3117, 2626, 2616, 3240, 2570, 3565, 2919, 3157, 2849, 3444, 2631, 3420, 2659, 3325, 3186, 2920, 3258, 3440, 3457, 3310, 2761, 3134, 2621, 3531, 2711, 2774, 2867, 2585, 3155, 2620, 3156, 2844, 3126, 3160, 2909, 3542, 3225, 2887, 3474, 3218, 2744, 2896, 3135, 3424, 3498, 3529, 2661, 2667, 2549, 2819, 2768, 2935, 3182, 2772, 3230, 3043, 2922, 3581, 3524, 2869, 2610, 3298, 2775, 3108, 2603, 3614, 2612, 2860, 3187, 3238, 3786, 4385, 3016, 3436, 2876, 2987, 3532, 3438, 3561, 3259, 2663, 2890, 3433, 3071, 3018, 2580, 2755, 3015, 2540, 3398, 2664, 2583, 2830, 2644, 2641, 3513, 2725, 2723, 3338, 3556, 2866, 2514, 2932, 2753, 2862, 2899, 2605, 2989, 3096, 3124, 2985, 2716, 3145, 3017, 2591, 2640, 3033, 1853, 3216, 3316, 3412, 2971, 3256, 3153, 3575, 3339, 2915, 2531, 3552, 3475, 3060, 3375, 2756, 3382, 2847, 2859, 3251, 3702, 3423, 2537, 2801, 3076, 2696, 3092, 3459, 3144, 3441, 2802, 3479, 2907, 3512, 3518, 3021, 2871, 3032, 2647, 2945, 3045, 2763, 3313, 3034, 3315, 3541, 2746, 2669, 2524, 3003, 2732, 3166, 3046, 3306, 2873, 3364, 3253, 3215, 3061, 2804, 2722, 2964, 2990, 2836, 4098, 4449, 3173, 2762, 3737, 2628, 3507, 2564, 3031, 2872, 3360, 2658, 2930, 2904, 3244, 3217, 3311, 3445, 2840, 3372, 2958, 3098, 3356, 3456, 3388, 2624, 3207, 2576, 2742, 2665, 2815, 3052, 3421, 2593, 3242, 2535, 2966, 2898, 2858, 2943, 2668, 2798, 3196, 2528, 2713, 3001, 2691, 2948, 2767, 2544, 3302, 2734, 2893, 2592, 2861, 3502, 2969, 2582, 3229, 2676, 2545, 3255, 3330, 2547, 3257, 2882, 3151, 3088, 2883, 3318, 2645, 2733, 2587, 3500, 3008, 2715, 2845, 3183, 2654, 3233, 3267, 2741, 2827, 2704, 3091, 2832, 2736, 3177, 3271, 2835, 2908, 3176, 2947, 3292, 3191, 2517, 3026, 3082, 3392, 3381, 2584, 2712, 3213, 3451, 2634, 3150, 2810, 3303, 2573, 3460, 2615, 3394, 2828, 3329, 4377, 4499, 3555, 2974, 3782, 3629, 3578, 3885, 2817, 3248, 2954, 3334, 3571, 3345, 3551, 3384, 3422, 1357, 1844, 2530, 2973, 4418, 4081, 3951, 1764, 1671, 1852, 334, 1843, 3449, 3185, 3399, 2786, 3055, 3472, 3397, 2601, 3564, 3277, 4373, 3548, 3179, 3407, 3841, 3347, 3142, 4255, 4431, 3241, 3077, 2700, 3147, 2648, 3283, 2829, 3278, 2581, 2536, 4363, 2848, 3410, 2797, 2523, 2520, 2721, 3496, 2571, 3198, 3219, 3030, 2673, 2921, 3837, 3301, 3322, 2705, 3239, 2949, 3369, 3396, 2892, 2897, 2766, 3174, 3297, 2550, 2889, 2933, 2793, 2627, 2677, 3119, 2596, 3226, 3172, 3089, 2868, 2839, 3942, 3120, 3286, 2843, 3465, 1564, 3946, 4222, 1504, 3999, 2682, 1875, 2865, 3539, 3168, 2864, 3308, 3557, 2940, 3109, 3503, 2625, 3455, 3114, 2595, 3331, 2978, 2518, 2521, 4079, 3024, 3573, 2952, 2653, 1769, 3180, 2813, 2652, 2600, 2816, 3201, 3188, 3270, 2997, 2992, 3009, 3520, 2936, 3275, 3165, 3447, 3131, 3305, 2809, 2792, 2729, 2814, 2619, 3266, 2825, 3066, 2857, 2998, 3064, 2681, 3035, 3279, 2764, 3480, 3127, 2912, 3010, 2607, 3211, 3111, 3041, 1573, 3909, 4269, 3487, 3489, 3019, 335, 1266, 1415, 2980, 3428, 2444, 3435, 3482, 3020, 2748, 2646, 2787, 2760, 2563, 3085, 3133, 3158, 3170, 2703, 3494, 2556, 2552, 3395, 3245, 3404, 2675, 2791, 2252, 4845, 1911, 4856, 908, 1935, 4711, 4728, 2460, 4517, 2749, 3341, 3355, 2575, 2942, 2886, 2758, 2594, 3495, 2884, 3516, 3042, 2737, 2923, 3563, 2855, 3476, 2611, 2937, 3383, 3497, 3402, 3162, 2662, 2875, 3223, 2878, 2079, 1808, 3285, 2757, 3057, 2951, 3519, 2579, 2853, 3387, 3401, 3493, 3393, 1608, 3365, 2885, 3574, 3081, 3007, 3568, 3152, 3367, 3358, 336, 2268, 3468, 3290, 3550, 3132, 2726, 2879, 3190, 3309, 2613, 2765, 3106, 1355, 3454, 2851, 2881, 3232, 3458, 3326, 3353, 2927, 3212, 2950, 3376, 3208, 3062, 3612, 3195, 3385, 2686, 2602, 3569, 3123, 2727, 1622, 2821, 2636, 2750, 2850, 3050, 2925, 3336, 3540, 2689, 3139, 2526, 3361, 3022, 3427, 3107, 3140, 3371, 3276, 2831, 3501, 2924, 3366, 3199, 3101, 3321, 3499, 2917, 2683, 2800, 2812, 3340, 3227, 3148, 3657, 4446, 1746, 1464, 1886, 2666, 3246, 3130, 3560, 3236, 2730, 3572, 1607, 3689, 2968, 1457, 1500, 909, 337, 1184, 3450, 2577, 2567, 2532, 3415, 2934, 2984, 2568, 4147, 3002, 2986, 910, 2979, 2542, 2492, 1762, 4472, 1472, 1529, 2319, 2131, 4057, 2048, 1976, 4507, 1583, 338, 2078, 2372, 339, 911, 1711, 340, 912, 2013, 913, 2277, 914, 341, 915, 4672, 4902, 916, 342, 1594, 4440, 917, 343, 344, 4559, 918, 2233, 919, 345, 920, 346, 921, 922, 347, 2226, 348, 2180, 2298, 349, 350, 2099, 2036, 351, 4808, 923, 924, 4498, 1518, 3728, 3637, 3570, 3429, 4091, 2870, 2995, 4338, 3205, 2880, 3261, 1994, 3968, 4291, 4204, 4071, 4034, 1742, 2820, 3912, 3287, 1218, 1190, 4145, 353, 2422, 354, 355, 925, 1782, 2184, 2261, 2490, 926, 927, 928, 4630, 2137, 3610, 356, 357, 4729, 358, 359, 4224, 1918, 1959, 1840, 3736, 2471, 1770, 929, 930, 931, 4155, 360, 361, 362, 932, 363, 364, 1279, 2289, 365, 1543, 366, 2156, 2472, 367, 4049, 1633, 1722, 368, 369, 1495, 1566, 370, 4967, 372, 373, 1211, 1873, 2101, 2494, 1726, 374, 1442, 933, 1748, 4142, 1434, 1358, 1191, 3732, 4110, 4039, 4411, 4506, 4270, 4156, 3780, 4036, 4030, 4016, 1550, 3899, 1247, 1855, 1181, 3919, 1639, 3664, 1244, 4140, 3930, 4117, 4189, 4001, 3799, 3643, 3619, 1240, 1686, 1479, 1307, 1575, 4296, 1665, 3871, 4135, 1605, 4408, 3604, 1274, 4149, 1313, 3741, 1938, 3709, 1727, 3893, 1280, 3622, 1784, 3834, 1752, 1983, 1743, 3979, 4461, 4113, 3817, 4232, 1340, 1489, 4214, 3902, 1857, 2316, 1904, 1219, 1486, 1512, 1481, 1516, 3820, 3789, 4252, 4047, 4330, 4082, 4094, 1574, 1248, 4078, 1872, 1426, 3785, 3687, 4318, 4243, 3866, 3704, 4172, 1621, 4315, 1192, 1513, 1856, 3324, 2769, 2678, 1561, 3296, 375, 2343, 376, 377, 1395, 1584, 4093, 4453, 3940, 3778, 4374, 4210, 4009, 4264, 4339, 4368, 1738, 378, 1610, 4194, 4474, 4013, 4266, 1771, 1759, 1922, 3860, 3832, 1349, 1381, 3663, 1642, 1185, 4356, 3932, 1233, 4212, 3858, 1198, 4192, 3611, 1919, 1819, 3790, 3712, 1969, 4095, 1887, 4468, 1720, 4247, 934, 1790, 2002, 1657, 1663, 1336, 3894, 1554, 1494, 1405, 3576, 4314, 3695, 1851, 1850, 4362, 1815, 3845, 4511, 3656, 3638, 1406, 1596, 4454, 1443, 1661, 935, 4504, 1359, 1734, 3650, 1910, 1305, 1648, 3831, 4435, 1208, 1988, 4451, 1993, 1986, 3849, 1718, 4068, 1835, 1425, 1428, 1282, 4380, 1658, 1452, 1687, 4024, 379, 4072, 3791, 4491, 4323, 4014, 1304, 1440, 1511, 1981, 1676, 1547, 4422, 1283, 3861, 1383, 3644, 3691, 4259, 1749, 1418, 1176, 4216, 1924, 1421, 3992, 4423, 4253, 1618, 1871, 1256, 3748, 4119, 1297, 1586, 1251, 1299, 1954, 4450, 3990, 4029, 4097, 3890, 3796, 1389, 3711, 4344, 1578, 1476, 4343, 1202, 4413, 4384, 3808, 1647, 1475, 3892, 3674, 1739, 3926, 3595, 1943, 3756, 4405, 3973, 4109, 4159, 3744, 4387, 1960, 4275, 3781, 1535, 1210, 4492, 4365, 4118, 3655, 3886, 4278, 4383, 4485, 1392, 3751, 3931, 3986, 3678, 1249, 4353, 4208, 3710, 4238, 4213, 3969, 4436, 3175, 4128, 2970, 1228, 3534, 3437, 3406, 3641, 3639, 3954, 4335, 3995, 3582, 4475, 3784, 4470, 4010, 3802, 3941, 4427, 4396, 4348, 3587, 3818, 3864, 3956, 3863, 3731, 3814, 4067, 4354, 4350, 4334, 3821, 1932, 3929, 4171, 1258, 3824, 3631, 4465, 4494, 1320, 1398, 3828, 1204, 1223, 1284, 1685, 3773, 4209, 3014, 4462, 4246, 4190, 4312, 4023, 3760, 3753, 1178, 3884, 2783, 4038, 3545, 2916, 3112, 2834, 3880, 3673, 4102, 4399, 4048, 3752, 4207, 1235, 3943, 4355, 4087, 4100, 4327, 4037, 4163, 3918, 4486, 1243, 3933, 4178, 1662, 1643, 4152, 3634, 3617, 4445, 4303, 1370, 3838, 4199, 4284, 1213, 4382, 1399, 1187, 3714, 1828, 4487, 3615, 1842, 1501, 3746, 4018, 4420, 3606, 4121, 3724, 3935, 4419, 4282, 1955, 1949, 4310, 4488, 1577, 4127, 1288, 1171, 3670, 4052, 3627, 3777, 936, 1371, 3685, 1325, 1940, 380, 4482, 4258, 3812, 1581, 1860, 4124, 4508, 4500, 3652, 3972, 1309, 1630, 1528, 3717, 1217, 3692, 4430, 3764, 4316, 4285, 3645, 3971, 4421, 1172, 4026, 2740, 3125, 3546, 1725, 1206, 3842, 4404, 3690, 3962, 4513, 4340, 4329, 4410, 3703, 4136, 1763, 1487, 3589, 3907, 4153, 2707, 3730, 4426, 4180, 4041, 3923, 4444, 3851, 4429, 4361, 1441, 4394, 4302, 3758, 4510, 3742, 1868, 4077, 1710, 1939, 1335, 1666, 1407, 4187, 1193, 1188, 1196, 1724, 1717, 1200, 1646, 1997, 1975, 1701, 4280, 1613, 4006, 1984, 1351, 3653, 3833, 1593, 2561, 4442, 3900, 4298, 1761, 4388, 1820, 1597, 4317, 4249, 1341, 1203, 1680, 4441, 3628, 3725, 1945, 937, 4206, 1588, 1439, 3794, 4447, 1838, 3776, 3658, 4064, 1377, 4011, 4272, 4503, 1324, 1367, 3594, 3680, 1300, 1750, 3913, 3774, 1877, 1987, 1862, 3998, 1380, 4185, 3686, 3696, 4463, 1350, 4008, 4230, 3853, 4244, 4083, 381, 1909, 3897, 1546, 382, 3613, 3273, 3452, 3525, 1229, 4105, 3835, 383, 1263, 1234, 3970, 1492, 3856, 3586, 1175, 4321, 4020, 4144, 1224, 1199, 3667, 2778, 4412, 4407, 1812, 1810, 3823, 1416, 1427, 1507, 1878, 4333, 3146, 4281, 4211, 1179, 3843, 1669, 1677, 1524, 1525, 1974, 384, 1979, 1456, 1239, 4167, 3740, 1876, 1388, 1967, 1474, 385, 1342, 1471, 4176, 1232, 1989, 4125, 1741, 1508, 1580, 4060, 4378, 1576, 1212, 1893, 3921, 3585, 1174, 1396, 4061, 4015, 1207, 3698, 3825, 3922, 4021, 4181, 1598, 1916, 1429, 4502, 4231, 1321, 4390, 3693, 3734, 1382, 3684, 1747, 3806, 3896, 3635, 1768, 4025, 3976, 3840, 3963, 4286, 1502, 3836, 4161, 3659, 3743, 4130, 3605, 1385, 1783, 4392, 4328, 1316, 4173, 4265, 1246, 1985, 1629, 1829, 1737, 3826, 1473, 1992, 1883, 1656, 1837, 1914, 1839, 1834, 3984, 4495, 3910, 4160, 3671, 4184, 4132, 4250, 1503, 1237, 1523, 1417, 4063, 1562, 3977, 1971, 1707, 1847, 1792, 3769, 4074, 1534, 4239, 4003, 3915, 1689, 1776, 1599, 1265, 1410, 1823, 1498, 1329, 939, 386, 940, 1655, 3874, 4261, 3626, 1791, 3579, 1644, 3936, 4054, 4260, 4359, 4131, 4297, 4357, 4022, 3770, 4046, 1222, 1996, 3788, 4169, 1925, 3953, 4228, 4226, 4277, 4101, 1889, 1369, 3797, 4088, 4019, 3771, 3632, 1402, 388, 1520, 1767, 1466, 1194, 4137, 1908, 1927, 1758, 3772, 3661, 4186, 4174, 1170, 3850, 1315, 1611, 1530, 1859, 2209, 4183, 1408, 1403, 941, 4033, 3623, 3596, 1461, 1848, 1173, 1928, 1459, 1765, 1826, 3719, 1931, 3584, 4004, 3591, 3967, 4168, 3957, 3878, 4165, 4191, 3980, 4058, 4200, 4336, 3958, 1260, 4051, 3798, 4032, 1774, 4322, 3991, 4106, 1641, 1570, 3978, 3965, 4464, 3813, 4154, 1365, 4257, 3846, 4345, 4379, 3603, 1693, 3937, 3801, 1787, 1214, 4483, 4201, 1898, 1845, 1226, 1220, 4245, 1347, 3867, 1900, 3640, 3916, 4089, 1209, 3745, 3988, 3975, 4236, 4075, 4146, 4256, 4308, 4000, 4148, 3588, 3868, 4403, 3883, 4326, 4393, 3895, 4237, 3955, 3727, 4005, 4092, 4372, 4283, 1553, 3805, 4151, 1281, 1681, 4489, 3982, 4437, 3597, 4342, 3872, 3987, 4490, 3876, 3668, 4300, 1679, 1242, 3598, 3583, 3647, 389, 4304, 3793, 3779, 1937, 1907, 4042, 3648, 3996, 3800, 4367, 4099, 1807, 3887, 4428, 1625, 1390, 1884, 1682, 4364, 4306, 3869, 1278, 1672, 942, 1505, 1542, 1709, 1915, 3600, 3914, 1968, 3948, 1670, 3811, 1865, 1362, 943, 1716, 3961, 1506, 4221, 1376, 944, 1683, 4276, 1445, 1721, 1632, 3607, 1328, 1455, 1913, 1497, 1317, 1951, 1778, 1379, 3768, 4225, 1668, 2383, 945, 390, 391, 4406, 4341, 1480, 392, 393, 394, 395, 1714, 1422, 396, 2043, 946, 2265, 397, 3618, 4433, 1177, 3898, 398, 2251, 947, 948, 2212, 949, 950, 399, 951, 400, 1965, 4640, 2129, 2345, 4471, 952, 953, 402, 2475, 403, 404, 4758, 955, 2437, 4139, 1482, 3602, 4434, 3721, 1592, 1825, 1698, 1519, 1352, 4202, 4409, 1977, 1917, 405, 406, 407, 408, 956, 2272, 957, 2267, 1631, 1684, 409, 2054, 3889, 958, 410, 2117, 411, 959, 412, 960, 2443, 4712, 4619, 2285, 414, 415, 961, 2352, 2417, 962, 1905, 416, 963, 417, 418, 3675, 1833, 3974, 419, 964, 421, 965, 966, 967, 968, 2365, 969, 970, 422, 1897, 2056, 2309, 4733, 1272, 4754, 2207, 971, 423, 4133, 3830, 4865, 972, 424, 973, 425, 2068, 4955, 426, 974, 427, 1708, 4676, 428, 4141, 430, 3749, 431, 1619, 975, 4044, 3590, 1944, 4126, 1923, 1706, 432, 976, 977, 978, 4867, 4400, 433, 434, 1606, 2143, 979, 435, 980, 2604, 2895, 981, 436, 3448, 4959, 2188, 437, 438, 1692, 982, 2388, 2433, 2243, 983, 2035, 2120, 4940, 439, 440, 2474, 442, 2052, 4764, 3862, 4055, 4358, 984, 2435, 4589, 2276, 2401, 985, 1664, 443, 2073, 2405, 986, 4309, 4045, 3854, 3682, 2491, 444, 445, 1972, 2688, 4366, 3425, 3231, 3649, 2759, 2724, 3873, 3755, 1216, 3599, 3775, 2754, 2606, 3471, 3234, 2565, 2803, 2562, 2684, 3411, 3405, 2690, 1255, 3694, 446, 3985, 3535, 1306, 4084, 3282, 447, 1353, 2168, 1780, 987, 988, 989, 2126, 448, 449, 2107, 450, 2031, 990, 451, 452, 991, 1485, 453, 992, 1728, 993, 4703, 994, 995, 454, 1696, 996, 2039, 455, 997, 998, 3116, 1869, 456, 4759, 2500, 4798, 2262, 4562, 457, 1386, 999, 458, 4768, 4982, 4778, 2040, 1000, 4981, 3879, 2698, 459, 460, 1001, 461, 1447, 462, 1002, 2042, 2339, 1003, 463, 464, 4425, 1645, 465, 1004, 467, 468, 469, 1231, 1468, 470, 471, 2496, 472, 473, 1005, 474, 475, 2108, 4725, 476, 1273, 1007, 1744, 1488, 1659, 477, 478, 4598, 1604, 4753, 2493, 479, 1008, 1009, 480, 1011, 1012, 1013, 1014, 481, 4313, 482, 483, 1499, 484, 1015, 4761, 2091, 2513, 1016, 4073, 1777, 2172, 485, 2205, 4592, 4545, 486, 1458, 4274, 2122, 4905, 5003, 4599, 4796, 2152, 4539, 4528, 4904, 4842, 4986, 4846, 4650, 4595, 2195, 4681, 2142, 487, 4710, 1775, 4205, 1526, 4586, 4602, 4518, 1697, 1017, 2000, 4692, 4795, 2480, 488, 2335, 489, 4969, 1894, 1882, 1817, 1880, 490, 1018, 3917, 3616, 4311, 1800, 4473, 4085, 4401, 1310, 3783, 3765, 1538, 3870, 491, 1948, 3699, 1735, 3792, 4290, 2074, 492, 2202, 5001, 2136, 493, 2211, 4532, 494, 2421, 495, 4090, 2378, 2104, 2210, 2247, 2478, 2047, 496, 4620, 4978, 4614, 1019, 2012, 1803, 3642, 497, 1322, 1020, 498, 1021, 2235, 2302, 2488, 4665, 4540, 5006, 499, 2049, 4685, 500, 501, 1022, 502, 1333, 503, 1023, 4686, 4804, 504, 4727, 505, 506, 4938, 4805, 2065, 1890, 4891, 1024, 507, 508, 509, 1025, 510, 1026, 3608, 1732, 511, 2397, 1864, 1816, 1531, 512, 513, 2451, 3636, 5012, 4892, 514, 1699, 4593, 1027, 4987, 515, 2495, 4660, 1028, 516, 1029, 2409, 1366, 2112, 2178, 2124, 2376, 5011, 4572, 4957, 4789, 4543, 4664, 4678, 2093, 4746, 2489, 4922, 2145, 1030, 4832, 4950, 1261, 4027, 3478, 3763, 2959, 1293, 2462, 517, 3197, 1595, 3413, 2238, 1031, 2228, 518, 2057, 2305, 2133, 2061, 4526, 4617, 1033, 2429, 2311, 1675, 4785, 1797, 519, 4578, 2271, 2466, 2148, 2420, 520, 4854, 2381, 2315, 2284, 4813, 521, 1034, 522, 523, 1999, 3759, 1215, 4122, 1730, 524, 525, 526, 3966, 4573, 5008, 1035, 527, 4973, 2508, 528, 3911, 529, 3289, 1821, 530, 531, 2426, 1036, 532, 1874, 533, 534, 1037, 535, 1038, 536, 1039, 537, 538, 539, 1040, 3767, 1929, 1042, 3683, 2100, 4853, 2300, 2165, 540, 541, 1043, 4906, 542, 1338, 2292, 2021, 1616, 1901, 1796, 1517, 4331, 4332, 4466, 2719, 3803, 4123, 4233, 1824, 3129, 3087, 3505, 4158, 4254, 1449, 1044, 3720, 4351, 4414, 1673, 3453, 3254, 2501, 1649, 543, 544, 545, 546, 1045, 547, 4240, 3580, 4680, 1046, 548, 2062, 1047, 550, 2598, 551, 552, 553, 3819, 1259, 4062, 1048, 554, 2841, 1049, 1050, 3515, 2219, 1433, 1323, 4301, 555, 556, 1051, 557, 558, 1052, 1053, 3738, 1567, 559, 1054, 1270, 3857, 1055, 2151, 560, 2175, 2089, 1056, 2198, 2193, 1257, 1238, 3662, 561, 2186, 1057, 4637, 562, 1058, 563, 4872, 2097, 564, 1059, 565, 566, 1060, 1061, 2231, 4203, 1899, 3981, 1062, 1303, 4116, 567, 1063, 568, 569, 570, 2367, 1946, 3620, 1064, 1514, 4002, 3679, 3964, 3707, 3666, 1348, 4273, 1804, 4497, 571, 1401, 4438, 1895, 1264, 572, 1912, 573, 3939, 4170, 4056, 1271, 1065, 1521, 1221, 1066, 1451, 1654, 1067, 3934, 4080, 4352, 574, 1700, 3739, 1565, 1419, 1811, 3950, 1690, 1397, 3716, 1569, 1465, 1437, 1674, 4337, 1285, 4268, 1183, 3726, 3881, 4134, 4347, 4012, 3938, 4066, 4198, 4416, 4164, 3630, 4229, 4292, 4193, 4325, 4375, 4371, 4432, 4215, 1753, 3944, 1409, 4195, 1729, 4223, 1540, 4455, 1068, 1069, 4512, 1723, 1070, 3750, 3633, 4070, 4104, 4162, 4235, 1863, 4346, 1881, 1814, 1572, 2418, 575, 4443, 3761, 1346, 1587, 1072, 3592, 576, 1891, 4469, 2430, 1275, 577, 1950, 1963, 4129, 1073, 1296, 1360, 1551, 3733, 4111, 3993, 4108, 3949, 1287, 1713, 1556, 1736, 1995, 1435, 1424, 1830, 1705, 1861, 1197, 1254, 4120, 1545, 4417, 1469, 1292, 2279, 1785, 1236, 4293, 1827, 3839, 3983, 4395, 1957, 2222, 578, 3928, 4295, 3715, 3855, 3609, 1990, 4076, 4175, 3927, 1757, 1650, 4299, 1773, 2004, 1267, 1364, 579, 1462, 1879, 4007, 3729, 3665, 3722, 1691, 1712, 1074, 1624, 1751, 1394, 3924, 3952, 580, 581, 2003, 1343, 2203, 1559, 1250, 1555, 582, 2395, 4114, 1818, 1075, 583, 1076, 1077, 1704, 4448, 584, 4882, 1078, 1079, 1786, 585, 3354, 1832, 586, 3432, 2891, 587, 588, 4583, 2505, 1903, 2077, 4936, 4658, 4830, 2446, 1080, 2278, 1081, 1082, 4459, 1083, 589, 1563, 1084, 2255, 2113, 590, 591, 1085, 592, 2308, 593, 594, 2274, 595, 4931, 2407, 1858, 596, 597, 598, 1453, 2559, 2590, 2738, 4478, 4069, 1809, 1327, 4150, 3260, 3481, 3672, 3625, 2008, 4439, 3852, 3997, 1411, 599, 3847, 4993, 1268, 4050, 3377, 4424, 1496, 4143, 4493, 3492, 1962, 1412, 2455, 600, 2457, 1086, 601, 4017, 4477, 1794, 2483, 2291, 602, 4263, 3072, 2009, 2199, 1754, 1087, 1088, 3888, 603, 604, 4777, 2324, 605, 1368, 3827, 4103, 606, 607, 1089, 2220, 4549, 1090, 1571, 1091, 1438, 608, 1092, 609, 610, 611, 1093, 612, 1094, 3701, 4389, 613, 4897, 1603, 614, 1095, 5000, 615, 616, 2263, 1332, 1715, 4369, 1652, 1096, 617, 1541, 618, 1921, 1733, 1373, 1870, 619, 4218, 1798, 2387, 1097, 1098, 1099, 1100, 620, 1702, 621, 622, 4801, 2398, 2239, 1101, 1667, 1384, 623, 4745, 3700, 1788, 1600, 624, 2098, 625, 4550, 4721, 4960, 4878, 2473, 4792, 4675, 1102, 1423, 626, 2771, 627, 1339, 3875, 628, 629, 3070, 4565, 2118, 4818, 2557, 2795, 2906, 2103, 4869, 2029, 3299, 3118, 4932, 4838, 4901, 4657, 4646, 4875, 4627, 4515, 4876, 4965, 4605, 4576, 2028, 4985, 4850, 1103, 4747, 1614, 1947, 2237, 630, 631, 4618, 4479, 3766, 632, 1104, 1105, 4886, 1106, 2432, 633, 1107, 4530, 634, 2371, 4651, 1108, 4536, 635, 1813, 636, 2431, 4866, 4991, 4814, 4560, 4956, 637, 4555, 1109, 4941, 4577, 2213, 638, 639, 3706, 1110, 640, 1111, 641, 4581, 4893, 4831, 2481, 4644, 4734, 2190, 4524, 4881, 4837, 4888, 4648, 642, 4807, 4896, 4868, 2374, 1112, 4823, 2164, 1113, 3735, 3390, 1694, 3431, 2838, 3483, 3464, 3848, 2558, 3314, 3086, 3029, 2956, 3517, 2651, 2914, 2680, 2632, 3121, 3100, 3528, 3526, 3084, 4220, 3304, 2846, 1186, 3235, 2903, 3559, 2687, 3409, 3237, 2900, 2999, 3349, 3171, 2752, 3262, 3504, 3047, 4376, 3193, 3430, 2525, 2670, 2911, 3508, 2776, 2697, 3200, 2790, 3323, 2543, 2874, 2614, 3904, 3373, 3054, 2617, 4242, 3621, 2708, 4179, 4398, 3959, 4935, 1269, 3095, 1286, 3624, 4481, 1432, 3511, 2735, 4381, 3295, 1585, 2390, 1568, 1114, 1766, 3221, 2643, 2739, 643, 4735, 4760, 1115, 4918, 4688, 3920, 644, 4723, 4949, 4748, 4607, 4719, 1116, 645, 646, 4611, 2353, 4645, 4998, 2307, 647, 1801, 4279, 3762, 1637, 1509, 4843, 4797, 4590, 4995, 4541, 2200, 4580, 4858, 648, 649, 2502, 1117, 4780, 4800, 4551, 4674, 1118, 1119, 2965, 2369, 2695, 3293, 650, 651, 4730, 4862, 4722, 4944, 1120, 652, 1121, 4983, 4784, 4877, 4643, 653, 654, 4909, 1122, 3264, 655, 2076, 4849, 656, 657, 2447, 658, 659, 4035, 2910, 3391, 2808, 3099, 2913, 3676, 2852, 3284, 2731, 1609, 2375, 2201, 3537, 3994, 1319, 4467, 1802, 1227, 1623, 660, 4844, 2086, 4770, 2192, 1123, 661, 2364, 662, 663, 664, 1124, 1125, 665, 2487, 4899, 2138, 1126, 666, 667, 4569, 4554, 4917, 2322, 1127, 1128, 1129, 668, 669, 1130, 670, 1131, 1527, 1132, 671, 1772, 2314, 1133, 1134, 2470, 1135, 672, 2236, 4716, 673, 4600, 2196, 2456, 4606, 4910, 2123, 1136, 1137, 2358, 5005, 2208, 4952, 4945, 1933, 1731, 1793, 675, 676, 3342, 1138, 677, 4597, 4870, 1140, 4990, 4954, 4916, 678, 1141, 4737, 4527, 2253, 4531, 679, 1142, 1143, 680, 4771, 1144, 4638, 681, 4705, 4525, 4522, 4898, 2325, 2415, 5004, 1145, 682, 683, 1146, 684, 685, 2445, 686, 687, 4861, 2157, 688, 3822, 689, 690, 4743, 691, 1147, 1601, 692, 1148, 693, 4537, 4825, 4625, 4621, 3905, 694, 695, 1414, 1149, 696, 2027, 2159, 4756, 4793, 4575, 2160, 4828, 4731, 4718, 4802, 4750, 1150, 2081, 2258, 697, 1795, 1372, 1695, 1866, 1151, 4115, 1719, 2280, 4662, 698, 2176, 2194, 2217, 1920, 699, 700, 701, 4999, 1544, 4739, 702, 703, 4031, 4305, 1636, 1393, 2010, 3810, 4452, 3677, 4227, 1277, 1400, 4402, 4157, 4197, 1337, 1253, 1532, 4028, 3989, 2449, 4715, 704, 2050, 1493, 1854, 1902, 1510, 1152, 4824, 705, 2340, 4182, 706, 2442, 2484, 707, 708, 709, 4667, 1153, 2412, 4476, 2053, 710, 1375, 711, 712, 713, 4829, 4738, 4811, 4570, 4961, 1245, 714, 2045, 4707, 715, 2169, 4713, 1841, 4234, 4924, 1154, 1314, 4096, 2023, 716, 717, 4895, 718, 1155, 719, 720, 2063, 1311, 721, 4642, 722, 4885, 4666, 2249, 4217, 1156, 723, 1157, 1627, 2116, 4566, 2287, 1158, 2330, 4826, 4622, 4740, 724, 4700, 725, 1159, 5013, 4894, 726, 2434, 1953, 1160, 1201, 727, 3787, 2046, 1161, 728, 729, 2485, 2044, 4568, 3747, 730, 731, 2024, 1846, 2507, 2368, 2019, 2333, 3815, 2005, 2266, 1964, 2347, 2385, 732, 2177, 2059, 4958, 1822, 4815, 1262, 2094, 4480, 1537, 1291, 2419, 1318, 2283, 4976, 4294, 2486, 4926, 1582, 2162, 733, 4587, 734, 735, 3439, 2833, 4287, 4509, 4505, 4267, 3697, 3272, 4112, 2967, 3651, 1991, 1805, 1628, 3705, 2479, 736, 1195, 4863, 1163, 3859, 1164, 2394, 1165, 2355, 2351, 4679, 4835, 2259, 4839, 4571, 1166, 4968, 2250, 4912, 2423, 737, 1167, 4989, 4684, 4669, 4552, 4612, 2127, 1745, 1973, 4767, 1168, 2273, 2310, 3807, 4307, 1169, 2303, 1454, or 1781.

37. The VP capsid polypeptide of claim 1, wherein X³, X⁶, and X¹ of SEQ ID NO: 5014 are basic amino acid residues.

38. The VP capsid polypeptide of claim 1, wherein X³, X⁶, and X⁴ of SEQ ID NO: 5014 are basic amino acid residues.

39. The VP capsid polypeptide of claim 37 or claim 38, wherein X³, X⁶, X¹, and X⁴ of SEQ ID NO: 5014 are basic amino acid residues.

40. A viral protein (VP) capsid polypeptide comprising a 581 to 589 region corresponding to residues 581 to 589 of an AAV5 VP1 polypeptide, wherein the 581 to 589 region of the VP capsid polypeptide comprises at least two basic amino acid residues and no more than one acidic amino acid residue.

41. The VP capsid polypeptide of claim 1, wherein the VP capsid polypeptide comprises one or more features selected from the group consisting of:
(a) the 581 to 589 region comprises two or more basic amino acid residues,
(b) X³, X⁶, X¹, or X⁴ of SEQ ID NO: 5014, or any combination thereof are basic amino acid residues, and
(c) the 581 to 589 region comprises no more than one acidic amino acid residue.

42. The VP capsid polypeptide of claim 41, wherein the VP capsid polypeptide comprises two or more of the features.

43. The VP capsid polypeptide of claim 41, wherein the VP capsid polypeptide comprises three of the features.

44. The VP capsid polypeptide of any one of claims 40-43, wherein the acidic amino acid residue is D or E.

45. The VP capsid polypeptide of any one of claims 37-44, wherein the basic amino acid residues are K, R, or H.

46. A viral protein (VP) capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 14-SEQ ID NO: 2013.

47. The VP capsid polypeptide of claim 46, wherein the 581 to 589 region comprises a sequence of any one of SEQ ID NO: 14-SEQ ID NO: 2013.

48. A viral protein (VP) capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 2514-SEQ ID NO: 4513.

49. The VP capsid polypeptide of claim 48, wherein the 581 to 589 region comprises a sequence of any one of SEQ ID NO: 2514-SEQ ID NO: 4513.

50. The VP capsid polypeptide of any one of claims 1-49, wherein the 581 to 589 region confers increased retina tissue tropism compared to a wild type AAV5 VP capsid polypeptide of SEQ ID NO: 1.

51. The VP capsid polypeptide of claim 50, wherein the retina tissue tropism is at least 1.5-fold, at least 2-fold, at least 2.5-fold, at least 3-fold, at least 3.5-fold, at least 4-fold, at least 5-fold, at least 10-fold, at least 20-fold, at least 50-fold, at least 75-fold, at least 100-fold, at least 200-fold, or at least 500-fold higher than the wild type AAV5 VP capsid polypeptide of SEQ ID NO: 1.

52. The VP capsid polypeptide of claim 50 or claim 51, wherein the 581 to 589 region further confers increased retinal pigment epithelium tissue tropism compared to a wild type AAV5 VP capsid polypeptide of SEQ ID NO: 1.

53. The VP capsid polypeptide of any one of claims 1-52, wherein the 581 to 589 region confers decreased photoreceptor tissue-tropism compared to a wild type AAV5 VP capsid polypeptide of SEQ ID NO: 1.

54. The VP capsid polypeptide of claim 53, wherein the photoreceptor tissue comprises rod photoreceptor cells, cone photoreceptor cells, or a combination thereof.

55. The VP capsid polypeptide of any one of claims 1-54, wherein the VP capsid polypeptide comprises an amino acid sequence at least 70%, at least 80%, at least 85%, at least 90%, at least 95%, at least 97%, at least 98%, at least 98.5%, at least 99%, or at least 99.5% identical to SEQ ID NO: 1.

56. The VP capsid polypeptide of any one of claims 1-55, wherein the VP capsid polypeptide has a sequence of SEQ ID NO: 2, wherein residues 581 to 589 of SEQ ID NO: 2 (X¹X²X³X⁴X⁵X⁶X⁷X⁸X⁹) correspond to the 581 to 589 region.

57. The VP capsid polypeptide of any one of claims 1-56, wherein the VP capsid polypeptide is capable of assembling into a recombinant viral capsid.

58. The VP capsid polypeptide of any one of claims 1-57, comprising at least one amino acid substitution as compared to each of SEQ ID NO: 3, SEQ ID NO: 4, SEQ ID NO: 5, SEQ ID NO: 6, SEQ ID NO: 7, and SEQ ID NO: 8.

59. The VP capsid polypeptide of any one of claims 1-58, wherein the 581 to 589 region comprises a net positive charge at pH 7.4.

60. The VP capsid polypeptide of any one of claims 1-59, wherein the VP capsid polypeptide further comprises one or more mutations outside of the 581 to 589 region relative to a wild type VP capsid polypeptide of SEQ ID NO: 1.

61. The VP capsid polypeptide of any one of claims 1-60, wherein the 581 to 589 region confers on a recombinant viral capsid assembled from the VP capsid polypeptide improved stability compared to a wild type AAV capsid comprising a peptide of SEQ ID NO: 1.

62. The VP capsid polypeptide of any one of claims 1-61, wherein the 581 to 589 region confers lower toxicity upon administration to a subject compared to a wild type VP capsid polypeptide of SEQ ID NO: 1.

63. The VP capsid polypeptide of any one of claims 1-62, wherein the VP capsid polypeptide further comprises one or more mutations outside of the 581 to 589 region that contributes to reduced production of neutralizing antibodies relative to a wild type VP capsid polypeptide of SEQ ID NO: 1.

64. The VP capsid polypeptide of any one of claims 1-63, wherein the VP capsid polypeptide further comprises one or more mutations outside of the 581 to 589 region that improves manufacturability relative to a wild type VP capsid polypeptide of SEQ ID NO: 1.

65. A recombinant viral adeno-associated virus (rAAV) capsid comprising the VP capsid polypeptide of any one of claims 1-64, wherein the rAAV capsid preferentially targets retina tissue.

66. The rAAV capsid of claim 65, further comprising a VP2 polypeptide comprising the 581 to 589 region and a VP3 polypeptide comprising the 581 to 589 region.

67. A recombinant adeno-associated virus (rAAV), comprising the VP capsid polypeptide of any one of claims 1-64 assembled into a recombinant viral capsid and a payload encapsidated by the recombinant viral capsid.

68. The rAAV of claim 67, wherein the rAAV preferentially targets retina tissue.

69. The rAAV of claim 67 or claim 68, further comprising a VP2 polypeptide comprising the 581 to 589 region and a VP3 polypeptide comprising the 581 to 589 region.

70. The rAAV of any one of claims 67-69, wherein the payload encodes a therapeutic polynucleotide or a therapeutic peptide.

71. The rAAV of claim 70, wherein the payload encodes a guide RNA, a tRNA, a suppressor tRNA, a siRNA, a miRNA, an mRNA, a shRNA, a circular RNA, or an antisense oligonucleotide (ASO), a ribozyme, a DNAzyme, an aptamer, or any combination thereof.

72. The rAAV of claim 71, wherein the guide RNA is a CRISPR/Cas guide RNA or an ADAR guide RNA.

73. The rAAV of claim 71, wherein the payload encodes a component of a CRISPR/Cas system, an adenosine deaminase acting on RNA (ADAR) enzyme, a transcriptional activator, or a transcriptional repressor.

74. A pharmaceutical composition comprising the VP capsid polypeptide of any one of claims 1-64, the rAAV capsid of claim 65 or claim 66, or the rAAV of any one of claims 67-73.

75. A method of delivering a payload to a retina tissue of a subject, the method comprising administering a recombinant adeno-associated virus (rAAV) to the subject via intravitreal administration, wherein the rAAV encapsidates the payload, and wherein the rAAV comprises a VP capsid polypeptide comprising a 581 to 589 region comprising at least one substitution relative to SEQ ID NO: 9, and wherein the 581 to 589 region comprises one or more basic amino acid residues.

76. A method of delivering a payload to a retina tissue of a subject, the method comprising administering a recombinant adeno-associated virus (rAAV) to the subject via intravitreal administration, wherein the rAAV encapsidates the payload, and wherein the rAAV comprises a VP capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 14-SEQ ID NO: 2013 or SEQ ID NO: 2514-SEQ ID NO: 4513.

77. A method of delivering a payload to a retina tissue of a subject, the method comprising administering a recombinant adeno-associated virus (rAAV) to the subject via intravitreal administration, wherein the rAAV encapsidates the payload, and wherein the rAAV comprises the VP capsid polypeptide of any one of claims 1-64.

78. A method of delivering a payload to a retina tissue of a subject, the method comprising:
(i) administering a recombinant adeno-associated virus (rAAV) to the subject, wherein the rAAV encapsidates the payload, and wherein the rAAV comprises a VP capsid polypeptide comprising a 581 to 589 region comprising at least one substitution relative to SEQ ID NO: 9, and wherein the 581 to 589 region comprises one or more basic amino acid residues; and
(ii) delivering the payload to the retina tissue.

79. A method of delivering a payload to a retina tissue of a subject, the method comprising:
(i) administering a recombinant adeno-associated virus (rAAV) to the subject, wherein the rAAV encapsidates the payload, and wherein the rAAV comprises a VP capsid polypeptide comprising a 581 to 589 region having at least 85% sequence identity to any one of SEQ ID NO: 14-SEQ ID NO: 2013 or SEQ ID NO: 2514-SEQ ID NO: 4513; and
(ii) delivering the payload to the retina tissue.

80. A method of delivering a payload to a retina tissue of a subject, the method comprising:
(i) administering a recombinant adeno-associated virus (rAAV) to the subject, wherein the rAAV encapsidates the payload, and wherein the rAAV comprises the VP capsid polypeptide of any one of claims 1-64; and
(ii) delivering the payload to the retina tissue.

81. The method of any one of claims 78-80, comprising administering the rAAV via intravitreal administration.

82. The method of any one of claims 78-80, comprising administering the rAAV via subretinal injection, topical mucosal administration, or systemic administration.

83. The method of any one of claims 75-82, further comprising expressing a therapeutic polypeptide or a therapeutic polynucleotide encoded by the payload.

84. The method of any one of claims 75-83, further comprising producing a therapeutic effect upon expression of the payload.

85. The method of claim 84, wherein the therapeutic effect is produced upon administration of from 1×10⁵ to 5×10¹⁴ rAAVs.

86. The method of claim 84 or claim 85, comprising administering an amount of the rAAV sufficient to produce the therapeutic effect without producing a toxicity in the subject.

87. A method of treating a condition in a subject, the method comprising administering a recombinant adeno-associated virus (rAAV) to the subject via intravitreal administration, wherein the rAAV encapsidates a payload, and wherein the rAAV comprises a VP capsid polypeptide comprising a 581 to 589 region comprising at least one substitution relative to SEQ ID NO: 9, and wherein the 581 to 589 region comprises one or more basic amino acid residues.

88. A method of treating a condition in a subject, the method comprising administering a recombinant adeno-associated virus (rAAV) to the subject via intravitreal administration, wherein the rAAV encapsidates a payload, and wherein the rAAV comprises a VP capsid polypeptide comprising a 581 to 589 region corresponding to residues 581 to 589 of an AAV5 VP1 polypeptide having at least 85% sequence identity to any one of SEQ ID NO: 14-SEQ ID NO: 2013 or SEQ ID NO: 2514-SEQ ID NO: 4513.

89. A method of treating a condition in a subject, the method comprising administering a recombinant adeno-associated virus (rAAV) to the subject via intravitreal administration, wherein the rAAV encapsidates a payload, and wherein the rAAV comprises the VP capsid polypeptide of any one of claims 1-64.

90. The method of any one of claims 87-89, further comprising expressing the payload in a retina tissue of the subject.

91. The method of any one of claims 87-90, further comprising producing a therapeutic effect in the subject.

92. A method of treating a condition in a subject, the method comprising:
(i) administering a recombinant adeno-associated virus (rAAV) to the subject, wherein the rAAV encapsidates a payload, and wherein the rAAV comprises a VP capsid polypeptide comprising a 581 to 589 region comprising at least one substitution relative to SEQ ID NO: 9, and wherein the 581 to 589 region comprises one or more basic amino acid residues; and
(ii) expressing the payload in a retina tissue of the subject, thereby treating the condition in the subject.

93. A method of treating a condition in a subject, the method comprising:
(i) administering a recombinant adeno-associated virus (rAAV) to the subject, wherein the rAAV encapsidates a payload, and wherein the rAAV comprises a VP capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 14-SEQ ID NO: 2013 or SEQ ID NO: 2514-SEQ ID NO: 4513; and
(ii) expressing the payload in a retina tissue of the subject, thereby treating the condition in the subject.

94. A method of treating a condition in a subject, the method comprising:
(i) administering a recombinant adeno-associated virus (rAAV) to the subject, wherein the rAAV encapsidates a payload, and wherein the rAAV comprises the VP capsid polypeptide of any one of claims 1-64; and
(ii) expressing the payload in a retina tissue of the subject, thereby treating the condition in the subject.

95. A method of treating a condition in a subject, the method comprising:
(i) administering a recombinant adeno-associated virus (rAAV) to the subject, wherein the rAAV encapsidates a payload, and wherein the rAAV comprises the VP capsid polypeptide of any one of claims 1-64;
(ii) delivering the payload to a retina tissue of the subject; and
(iii) producing a therapeutic effect in the retina tissue, thereby treating the condition.

96. The method of any one of claims 92-95, comprising administering the rAAV via intravitreal administration.

97. The method of any one of claims 92-95, comprising administering the rAAV via subretinal injection, topical mucosal administration, or systemic administration.

98. The method of any one of claims 87-97, wherein the condition is an ocular condition.

99. The method of claim 98, wherein the ocular condition is achromatopsia, macular degeneration, cataracts, choroideremia, glaucoma, optical neuropathy, Marfan syndrome, myopia, polypoidal choroidal vasculopathy, retinitis pigmentosa, Stargardt disease, Usher syndrome, Leber congenital amaurosis, Leber hereditary optical neuropathy, Uveal melanoma, or X-linked retinoschisis.

100. The method of claim 98 or claim 99, wherein the ocular condition is Stargardt disease.

101. The method of any one of claims 76-100, further comprising delivering the rAAV to a retina tissue with higher tissue tropism than a wild type AAV5 capsid comprising a peptide of SEQ ID NO: 1.

102. The method of any one of claims 76-101, further comprising delivering the rAAV to a retina tissue with higher tissue tropism than for photoreceptor tissue.

103. The method of any one of claims 76-102, comprising delivering the rAAV to a retina tissue with at least 2.5-fold, at least 3-fold, at least 3.5-fold, at least 4-fold, at least 5-fold, at least 10-fold, at least 20-fold, at least 50-fold, at least 75-fold, at least 100-fold, at least 200-fold, at least 500-fold higher, or at least 1000-fold higher tissue tropism than a wild type AAV5 capsid comprising a peptide of SEQ ID NO: 1.

104. The method of any one of claims 76-103, comprising administering from 1×10⁵ to 5×10¹⁴ rAAVs.

105. The method of any one of claims 76-104, wherein the payload encodes a therapeutic protein, therapeutic polynucleotide, a guide RNA, a tRNA, a suppressor tRNA, a siRNA, a miRNA, an mRNA, a shRNA, a circular RNA, an antisense oligonucleotide (ASO), a ribozyme, a DNAzyme, an aptamer, or any combination thereof.

106. The method of claim 105, wherein the guide RNA is a CRISPR/Cas guide RNA or an ADAR guide RNA.

107. The method of claim 105, wherein the therapeutic protein is selected from the group consisting of CNGB3, NOS2A, CFH, CF, C2, C3, CFB, HTRA1/LOC, MMP-9, TIMP-3, SLC16A8, GEMIN4, CYP51A1, RIC1, TAPT1, TAF1A, WDR87, APE1, MIP, Cx50, GJA3, GJA8, CRYAA, CRYBB2, PRX, POLR3B, XRCC1, ZNF350, EPHA2, REP1, CALM2, MPP-7, Optineurin, LOX1, CYP1B1, CAV1/2, MYOC, PITX2, FOXC1, PAX6, LTBP2, Complex I, ND, OPA1, RPE65, FBN1, TGFBR2, MTHFR, MTR, MTRR, HGF, C-MET, UMODL1, MMP-1/2, CBS, IGF-1, UHRF1BP1L, PTPRR, PPFIA2, P4HA2, SERPING1, PEDF, ARMS2-HTRA1, FGD6, ABCG1, LOC387715, CETP, NRL, RDH12, PRPH2 (RDS), RHO, RPGR, SNRNP200, NR2E3, IMPDH1, CRX, HK1, IMPDH2, PRPF3, AGBL5, ABCA1, ABCA4, CRB1, USH2A, NRP1, ND4, RLBP1, PTEN, BAP1, GNAQ, GNA11, DDEF1, SF3B1, EIF1AX, CDKN2A, p14ARF, HERC2/OCA2, VEGF, and RS1.

108. The method of claim 105, wherein the therapeutic polynucleotide targets an mRNA encoding a protein selected from the group consisting of CNGB3, NOS2A, CFH, CF, C2, C3, CFB, HTRA1/LOC, MMP-9, TIMP-3, SLC16A8, GEMIN4, CYP51A1, RIC1, TAPT1, TAF1A, WDR87, APE1, MIP, Cx50, GJA3, GJA8, CRYAA, CRYBB2, PRX, POLR3B, XRCC1, ZNF350, EPHA2, REP1, CALM2, MPP-7, Optineurin, LOX1, CYP1B1, CAV1/2, MYOC, PITX2, FOXC1, PAX6, LTBP2, Complex I, ND, OPA1, RPE65, FBN1, TGFBR2, MTHFR, MTR, MTRR, HGF, C-MET, UMODL1, MMP-1/2, CBS, IGF-1, UHRF1BP1L, PTPRR, PPFIA2, P4HA2, SERPING1, PEDF, ARMS2-HTRA1, FGD6, ABCG1, LOC387715, CETP, NRL, RDH12, PRPH2 (RDS), RHO, RPGR, SNRNP200, NR2E3, IMPDH1, CRX, HK1, IMPDH2, PRPF3, AGBL5, ABCA1, ABCA4, CRB1, USH2A, NRP1, ND4, RLBP1, PTEN, BAP1, GNAQ, GNA11, DDEF1, SF3B1, EIF1AX, CDKN2A, p14ARF, HERC2/OCA2, VEGF, or RS1.

109. The method of claim 105, wherein the payload encodes a therapeutic polynucleotide targeting ABCA1, ABCA4, or CRB1 or a transgene encoding ABCA1, ABCA4, or CRB1.

110. The method of claim 105, wherein the payload encodes a component of a CRISPR/Cas system, an adenosine deaminase acting on RNA (ADAR) enzyme, a transcriptional activator, or a transcriptional repressor.

111. The method of claim 110, wherein the component of the CRISPR/Cas system comprises a Cas3, a Cas8, a Cas10, a Cas9, a Cas4, a Cas12, a Cas13, a guide RNA, or a combination thereof.

112. The method of claim 110, wherein the ADAR enzyme is ADAR1 or ADAR2.

113. The method of claim 110, wherein the transcriptional activator is VP64.

114. The method of claim 110, wherein the transcriptional repressor is KRAB.

115. The method of any one of claims 105-114, further comprising expressing a therapeutic polypeptide or a therapeutic polynucleotide encoded by the payload.

116. The method of any one of claims 105-115, comprising expressing the therapeutic polypeptide or the therapeutic polynucleotide at a higher level in a retina tissue compared to a wild type AAV5 capsid comprising a peptide of SEQ ID NO: 1.

117. The method of claim 116, comprising expressing the therapeutic polypeptide or the therapeutic polynucleotide in a retina tissue at a level that is at least 3-fold, at least 3.5-fold, at least 4-fold, at least 5-fold, at least 10-fold, at least 20-fold, at least 50-fold, at least 75-fold, at least 100-fold, at least 200-fold, at least 500-fold higher, or at least 1000-fold higher compared to the wild type AAV5 capsid.

118. The method of any one of claims 76-117, comprising producing a toxicity in the subject that is lower than a toxicity produced upon administration of a comparable number of wild type AAV5 capsids comprising a VP capsid polypeptide of SEQ ID NO: 1.

119. The method of any one of claims 76-118, comprising producing a toxicity in the subject that is lower than a toxicity produced upon administration of a number of wild type AAV5 capsids comprising a VP capsid polypeptide of SEQ ID NO: 1 sufficient to deliver a comparable number of payloads to a retina tissue.

120. The method of any one of claims 76-119, comprising producing a concentration of neutralizing antibodies in the subject that is lower than a concentration of neutralizing antibodies produced upon administration of a comparable number of wild type AAV5 capsids comprising a VP capsid polypeptide of SEQ ID NO: 1.

121. The method of any one of claims 76-120, comprising producing a concentration of neutralizing antibodies in the subject that is lower than a concentration of neutralizing antibodies produced upon administration of a comparable number of wild type AAV2 capsids.

122. The method of any one of claims 76-121, comprising producing a concentration of neutralizing antibodies in the subject that is lower than a concentration of neutralizing antibodies produced upon administration of a comparable number of wild type AAV8 capsids.

123. The method of any one of claims 76-122, comprising producing a concentration of neutralizing antibodies in the subject that is lower than a concentration of neutralizing antibodies produced upon administration of a comparable number of wild type AAV9 capsids.

124. The method of any one of claims 76-123, comprising producing a concentration of neutralizing antibodies in the subject that is lower than a concentration of neutralizing antibodies produced upon administration of a number of wild type AAV5 capsids comprising a VP capsid polypeptide of SEQ ID NO: 1 sufficient to deliver a comparable number of payloads to a retina tissue.

125. The rAAV capsid of claim 65 or 66, the rAAV of any one of claims 67 to 73, or the pharmaceutical composition of claim 74 for use as a medicament.

126. The rAAV capsid of claim 65 or 66, the rAAV of any one of claims 67 to 73, or the pharmaceutical composition of claim 74 for use in a method of treating an ocular condition.

127. The rAAV capsid, rAAV or pharmaceutical composition for use of claim 126, wherein the ocular condition is achromatopsia, macular degeneration, cataracts, choroideremia, glaucoma, optical neuropathy, Marfan syndrome, myopia, polypoidal choroidal vasculopathy, retinitis pigmentosa, Stargardt disease, Usher syndrome, Leber congenital amaurosis, Leber hereditary optical neuropathy, Uveal melanoma, or X-linked retinoschisis.

128. The rAAV capsid, rAAV or pharmaceutical composition for use of claim 126 or 127, wherein the ocular condition is Stargardt disease.

129. A viral protein (VP) capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 2014-SEQ ID NO: 2513.

130. The VP capsid polypeptide of claim 1, wherein the 581 to 589 region comprises a sequence of any one of SEQ ID NO: 2014-SEQ ID NO: 2513.

131. A viral protein (VP) capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 4514-SEQ ID NO: 5013.

132. The VP capsid polypeptide of claim 3, wherein the 581 to 589 region comprises a sequence of any one of SEQ ID NO: 4514-SEQ ID NO: 5013.

133. A recombinant viral adeno-associated virus (rAAV) capsid comprising the VP capsid polypeptide of any one of claims 129-132, wherein the rAAV capsid preferentially targets retinal pigment epithelium tissue.

134. A recombinant viral adeno-associated virus (rAAV) capsid comprising the VP capsid polypeptide of any one of claims 129-132, wherein the rAAV capsid preferentially targets choroid tissue.

135. A method of delivering a payload to a retinal pigment epithelium tissue of a subject, the method comprising administering a recombinant adeno-associated virus (rAAV) to the subject via intravitreal administration, wherein the rAAV encapsidates the payload, and wherein the rAAV comprises a VP capsid polypeptide comprising a 581 to 589 region corresponding to residues 581 to 589 of an AAV5 VP1 polypeptide having at least 85% sequence identity to any one of SEQ ID NO: 2014-SEQ ID NO: 2513 or SEQ ID NO: 4514-SEQ ID NO: 5013.

136. A method of delivering a payload to a retinal pigment epithelium tissue of a subject, the method comprising administering a recombinant adeno-associated virus (rAAV) to the subject via intravitreal administration, wherein the rAAV encapsidates the payload, and wherein the rAAV comprises a VP capsid polypeptide comprising a 581 to 589 region corresponding to residues 581 to 589 of an AAV5 VP1 polypeptide having at least 85% sequence identity to any one of SEQ ID NO: 2014-SEQ ID NO: 2513 or SEQ ID NO: 4514-SEQ ID NO: 5013.

137. A method of delivering a payload to a choroid tissue of a subject, the method comprising:
(i) administering a recombinant adeno-associated virus (rAAV) to the subject, wherein the rAAV encapsidates the payload, and wherein the rAAV comprises a VP capsid polypeptide comprising a 581 to 589 region having at least 85% sequence identity to any one of SEQ ID NO: 2014-SEQ ID NO: 2513 or SEQ ID NO: 4514-SEQ ID NO: 5013; and
(ii) delivering the payload to the choroid tissue.

138. A method of delivering a payload to a retinal pigment epithelium tissue of a subject, the method comprising:
(i) administering a recombinant adeno-associated virus (rAAV) to the subject, wherein the rAAV encapsidates the payload, and wherein the rAAV comprises a VP capsid polypeptide comprising a 581 to 589 region having at least 85% sequence identity to any one of SEQ ID NO: 2014-SEQ ID NO: 2513 or SEQ ID NO: 4514-SEQ ID NO: 5013; and
(ii) delivering the payload to the retinal pigment epithelium tissue.

139. A method of treating a condition in a subject, the method comprising administering a recombinant adeno-associated virus (rAAV) to the subject via intravitreal administration, wherein the rAAV encapsidates a payload, and wherein the rAAV comprises a VP capsid polypeptide comprising a 581 to 589 region corresponding to residues 581 to 589 of an AAV5 VP1 polypeptide having at least 85% sequence identity to any one of SEQ ID NO: 2014-SEQ ID NO: 2513 or SEQ ID NO: 4514-SEQ ID NO: 5013.

140. A method of treating a condition in a subject, the method comprising:
(i) administering a recombinant adeno-associated virus (rAAV) to the subject, wherein the rAAV encapsidates a payload, and wherein the rAAV comprises a VP capsid polypeptide comprising a 581 to 589 region corresponding to residues 581 to 589 of an AAV5 VP1 polypeptide, wherein the 581 to 589 region comprises a sequence having at least 85% sequence identity to any one of SEQ ID NO: 2014-SEQ ID NO: 2513 or SEQ ID NO: 4514-SEQ ID NO: 5013; and
(ii) expressing the payload in a retinal pigment epithelium tissue of the subject, thereby treating the condition in the subject.
